(19) United States
(12) Patent Application Publication
(10) Pub. No.: US 2024/0074311 A1
(43) Pub. Date: Feb. 29, 2024

(54) COMPOUND, ORGANIC ELECTROLUMINESCENT ELEMENT AND ELECTRONIC DEVICE

(71) Applicant: IDEMITSU KOSAN CO.,LTD., Tokyo (JP)

(72) Inventors: Shintaro BAN, Chiyoda-ku (JP); Yu KUDO, Chiyoda-ku (JP); Kei YOSHIDA, Chiyoda-ku (JP); Sigma HASHIMOTO, Chiyoda-ku (JP); Yoshinao SHIRASAKI, Chiyoda-ku (JP)

(73) Assignee: IDEMITSU KOSAN CO.,LTD., Tokyo (JP)

(21) Appl. No.: 18/333,862

(22) Filed: Jun. 13, 2023

(30) Foreign Application Priority Data
Jun. 15, 2022 (JP) 2022-096824

(51) Int. Cl. H10K 85/60; C07D 307/77; C09K 11/06

(52) U.S. Cl. CPC H10K 85/6574; C07D 307/77; C09K 11/06; H10K 85/622; H10K 85/626; H10K 50/13

(57) ABSTRACT

A compound is represented by a formula (1) below. In the formula (1): R₁ to R₉, R₁₀₁ to R₁₀₈, and R₁₁₁ to R₁₁₈ are each independently a hydrogen atom, a substituted or unsubstituted aryl group having 6 to 20 ring carbon atoms, or the like; Ar₁₂ is a substituted or unsubstituted aryl group having 10 to 30 ring carbon atoms or the like; p is 0 or 1; q is 0 or 1; and p+q is 1 or 2.

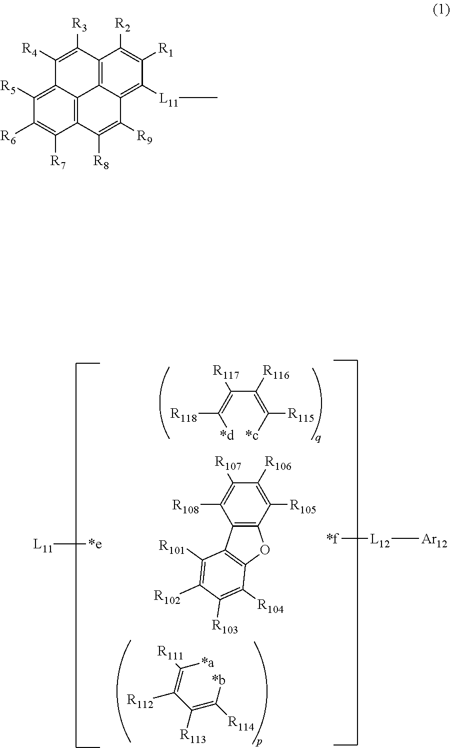

The entire disclosure of Japanese Patent Application No. 2022-096824, filed Jun. 15, 2022, is expressly incorporated by reference herein.

### TECHNICAL FIELD

The present invention relates to a compound, an organic electroluminescence device, and an electronic device.

### BACKGROUND ART

An organic electroluminescence device (hereinafter, occasionally referred to as “organic EL device”) has found its application in a full-color display for mobile phones, televisions, and the like. When voltage is applied to an organic EL device, holes are injected from an anode and electrons are injected from a cathode into an emitting layer. The injected holes and electrons are recombined in the emitting layer to form excitons. Specifically, according to the electron spin statistics theory, singlet excitons and triplet excitons are generated at a ratio of 25%:75%.

In order to enhance the performance of the organic EL device, studies have been made for compounds (e.g., pyrene compounds) used for the organic EL device in, for instance, International Publication No. 2021/049663, Chinese Patent Application Publication No. 104650029, European Patent Application Publication No. 2463352, U.S. Patent Application Publication No. 2009/0131673, and U.S. Patent Application Publication No. 2006/0240283. In addition, in order to enhance the performance of the organic EL device, International Publication No. 2010/134350 describes a phenomenon in which a singlet exciton is generated by collision and fusion of two triplet excitons (hereinafter, occasionally referred to as a Triplet-Triplet Fusion (TTF) phenomenon).

The performance of the organic EL device is evaluable in terms of, for instance, luminance, emission wavelength, chromaticity, luminous efficiency, drive voltage, and lifetime.

For blue fluorescent organic electroluminescence devices, improvement in lifetime and inhibition of deterioration in chromaticity have been demanded.

### SUMMARY OF THE INVENTION

An object of the invention is to provide a compound that can provide an organic electroluminescence device with a prolonged lifetime and improved chromaticity. Another object of the invention is to provide an organic electroluminescence device having a long lifetime and improved chromaticity and to provide an electronic device including the organic electroluminescence device.

According to an aspect of the invention, a compound represented by a formula (1) below is provided.

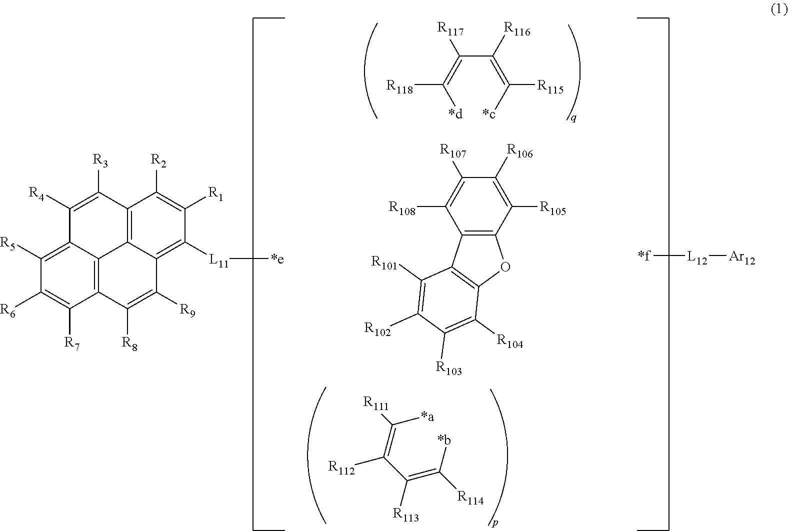

In the formula (1):
R₁ to R₉, R₁₀₁ to R₁₀₈, and R₁₁₁ to R₁₁₈ are each independently a hydrogen atom, a substituted or unsubstituted aryl group having 6 to 20 ring carbon atoms, or a substituted or unsubstituted heterocyclic group having 5 to 21 ring atoms;
Ar₁₂ is a substituted or unsubstituted aryl group having 10 to 30 ring carbon atoms, or a substituted or unsubstituted heterocyclic group having 9 to 31 ring atoms;
the substituted aryl group represented by Ar₁₂ has or does not have, as a substituent, at least one group selected from the group consisting of a substituted or unsubstituted aryl group having 6 to 20 ring carbon atoms and a substituted or unsubstituted heterocyclic group having 5 to 21 ring atoms;
the substituted heterocyclic group represented by Ar₁₂ has or does not have, as a substituent, at least one selected from the group consisting of a substituted or unsubstituted aryl group having 6 to 20 ring carbon atoms and a substituted or unsubstituted heterocyclic group having 5 to 21 ring atoms;
L₁₁ and L₁₂ are each independently a single bond, a substituted or unsubstituted arylene group having 6 to 10 ring carbon atoms, or a substituted or unsubstituted divalent heterocyclic group having 5 to 13 ring atoms;
p is 0 or 1;
q is 0 or 1; and
p+q is 1 or 2,
when p is 1, one of R₁₀₁ and R₁₀₂, R₁₀₂ and R₁₀₃, or R₁₀₃ and R₁₀₄ is a single bond bonded to *a, and the other of R₁₀₁ and R₁₀₂, R₁₀₂ and R₁₀₃, or R₁₀₃ and R₁₀₄ is a single bond bonded to *b;
when q is 0, of two selected from R₁₀₅ to R₁₀₈, one is a single bond bonded to *e, and the other is a single bond bonded to *f; and
when q is 1, one of R₁₀₅ and R₁₀₆, R₁₀₆ and R₁₀₇, or R₁₀₇ and R₁₀₈ is a single bond bonded to *c, and the other of R₁₀₅ and R₁₀₆, R₁₀₆ and R₁₀₇, or R₁₀₇ and R₁₀₈ is a single bond bonded to *d, and of two selected from R₁₀₅ to R₁₀₈ not being the single bonds bonded to *c and *d and R₁₁₅ to R₁₁₈, one is a single bond bonded to *e, and the other is a single bond bonded to *f.

According to an aspect of the invention, there is provided an organic electroluminescence device including an anode, a cathode, and an emitting zone provided between the anode and the cathode, in which the emitting zone includes a first emitting layer that contains a compound according to an aspect of the invention as a first host material.

According to an aspect of the invention, there is provided an electronic device including an organic electroluminescence device according to an aspect of the invention.

According to an aspect of the invention, there is provided a compound that can provide an organic electroluminescence device with a prolonged lifetime and improved chromaticity. According to an aspect of the invention, there is provided an organic electroluminescence device having a long lifetime and improved chromaticity. According to an aspect of the invention, there is provided an electronic device including the organic electroluminescence device.

### BRIEF DESCRIPTION OF DRAWINGS

FIG. 1 schematically depicts an exemplary arrangement of an organic electroluminescence device according to an exemplary embodiment of the invention.

FIG. 2 schematically depicts another exemplary arrangement of the organic electroluminescence device according to an exemplary embodiment of the invention.

FIG. 3 schematically depicts still another exemplary arrangement of the organic electroluminescence device according to an exemplary embodiment of the invention.

### DESCRIPTION OF EMBODIMENT(S)

### Definitions

Herein, a hydrogen atom includes isotope having different numbers of neutrons, specifically, protium, deuterium and tritium.

In chemical formulae herein, it is assumed that a hydrogen atom (i.e. protium, deuterium and tritium) is bonded to each of bondable positions that are not annexed with signs “R” or the like or “D” representing a deuterium.

Herein, the ring carbon atoms refer to the number of carbon atoms among atoms forming a ring of a compound (e.g., a monocyclic compound, fused-ring compound, cross-linking compound, carbon ring compound, and heterocyclic compound) in which the atoms are bonded to each other to form the ring.

When the ring is substituted by a substituent(s), carbon atom(s) contained in the substituent(s) is not counted in the ring carbon atoms.

Unless otherwise specified, the same applies to the “ring carbon atoms” described later.

For instance, a benzene ring has 6 ring carbon atoms, a naphthalene ring has 10 ring carbon atoms, a pyridine ring has 5 ring carbon atoms, and a furan ring has 4 ring carbon atoms.

Further, for instance, 9,9-diphenylfluorenyl group has 13 ring carbon atoms and 9,9′-spirobifluorenyl group has 25 ring carbon atoms.

When a benzene ring is substituted by a substituent in a form of, for instance, an alkyl group, the number of carbon atoms of the alkyl group is not counted in the number of the ring carbon atoms of the benzene ring.

Accordingly, the benzene ring substituted by an alkyl group has 6 ring carbon atoms.

When a naphthalene ring is substituted by a substituent in a form of, for instance, an alkyl group, the number of carbon atoms of the alkyl group is not counted in the number of the ring carbon atoms of the naphthalene ring.

Accordingly, the naphthalene ring substituted by an alkyl group has 10 ring carbon atoms.

Herein, the ring atoms refer to the number of atoms forming a ring of a compound (e.g., a monocyclic compound, fused-ring compound, cross-linking compound, carbon ring compound, and heterocyclic compound) in which the atoms are bonded to each other to form the ring (e.g., monocyclic ring, fused ring, and ring assembly).

Atom(s) not forming the ring (e.g., hydrogen atom(s) for saturating the valence of the atom which forms the ring) and atom(s) in a substituent by which the ring is substituted are not counted as the ring atoms.

Unless otherwise specified, the same applies to the “ring atoms” described later.

For instance, a pyridine ring has 6 ring atoms, a quinazoline ring has 10 ring atoms, and a furan ring has 5 ring atoms.

For instance, the number of hydrogen atom(s) bonded to a pyridine ring or the number of atoms forming a substituent is not counted as the pyridine ring atoms.

Accordingly, a pyridine ring bonded to a hydrogen atom(s) or a substituent(s) has 6 ring atoms.

For instance, the hydrogen atom(s) bonded to carbon atom(s) of a quinazoline ring or the atoms forming a substituent are not counted as the quinazoline ring atoms.

Accordingly, a quinazoline ring bonded to hydrogen atom(s) or a substituent(s) has 10 ring atoms.

Herein, “XX to YY carbon atoms” in the description of “substituted or unsubstituted ZZ group having XX to YY carbon atoms” represent carbon atoms of an unsubstituted ZZ group and do not include carbon atoms of a substituent(s) of the substituted ZZ group.

Herein, “YY” is larger than “XX,” “XX” representing an integer of 1 or more and “YY” representing an integer of 2 or more.

Herein, “XX to YY atoms” in the description of “substituted or unsubstituted ZZ group having XX to YY atoms” represent atoms of an unsubstituted ZZ group and does not include atoms of a substituent(s) of the substituted ZZ group.

Herein, “YY” is larger than “XX,” “XX” representing an integer of 1 or more and “YY” representing an integer of 2 or more.

Herein, an unsubstituted ZZ group refers to an “unsubstituted ZZ group” in a “substituted or unsubstituted ZZ group,” and a substituted ZZ group refers to a “substituted ZZ group” in a “substituted or unsubstituted ZZ group.”

Herein, the term “unsubstituted” used in a “substituted or unsubstituted ZZ group” means that a hydrogen atom(s) in the ZZ group is not substituted with a substituent(s).

The hydrogen atom(s) in the “unsubstituted ZZ group” is protium, deuterium, or tritium.

Herein, the term “substituted” used in a “substituted or unsubstituted ZZ group” means that at least one hydrogen atom in the ZZ group is substituted with a substituent.

Similarly, the term “substituted” used in a “BB group substituted by AA group” means that at least one hydrogen atom in the BB group is substituted with the AA group.

### Substituent Mentioned Herein

Substituent mentioned herein will be described below.

An “unsubstituted aryl group” mentioned herein has, unless otherwise specified herein, 6 to 50, preferably 6 to 30, more preferably 6 to 18 ring carbon atoms.

An “unsubstituted heterocyclic group” mentioned herein has, unless otherwise specified herein, 5 to 50, preferably 5 to 30, more preferably 5 to 18 ring atoms.

An “unsubstituted alkyl group” mentioned herein has, unless otherwise specified herein, 1 to 50, preferably 1 to 20, more preferably 1 to 6 carbon atoms.

An “unsubstituted alkenyl group” mentioned herein has, unless otherwise specified herein, 2 to 50, preferably 2 to 20, more preferably 2 to 6 carbon atoms.

An “unsubstituted alkynyl group” mentioned herein has, unless otherwise specified herein, 2 to 50, preferably 2 to 20, more preferably 2 to 6 carbon atoms.

An “unsubstituted cycloalkyl group” mentioned herein has, unless otherwise specified herein, 3 to 50, preferably 3 to 20, more preferably 3 to 6 ring carbon atoms.

An “unsubstituted arylene group” mentioned herein has, unless otherwise specified herein, 6 to 50, preferably 6 to 30, more preferably 6 to 18 ring carbon atoms.

An “unsubstituted divalent heterocyclic group” mentioned herein has, unless otherwise specified herein, 5 to 50, preferably 5 to 30, more preferably 5 to 18 ring atoms.

An “unsubstituted alkylene group” mentioned herein has, unless otherwise specified herein, 1 to 50, preferably 1 to 20, more preferably 1 to 6 carbon atoms.

### Substituted or Unsubstituted Aryl Group

Specific examples (specific example group G1) of the “substituted or unsubstituted aryl group” mentioned herein include unsubstituted aryl groups (specific example group G1A) below and substituted aryl groups (specific example group G1B).

(Herein, an unsubstituted aryl group refers to an “unsubstituted aryl group” in a “substituted or unsubstituted aryl group”, and a substituted aryl group refers to a “substituted aryl group” in a “substituted or unsubstituted aryl group.”)

A simply termed “aryl group” herein includes both of an “unsubstituted aryl group” and a “substituted aryl group”.

The “substituted aryl group” refers to a group derived by substituting at least one hydrogen atom in an “unsubstituted aryl group” with a substituent.

Examples of the “substituted aryl group” include a group derived by substituting at least one hydrogen atom in the “unsubstituted aryl group” in the specific example group G1A below with a substituent, and examples of the substituted aryl group in the specific example group G1B below.

It should be noted that the examples of the “unsubstituted aryl group” and the “substituted aryl group” mentioned herein are merely exemplary, and the “substituted aryl group” mentioned herein includes a group derived by further substituting a hydrogen atom bonded to a carbon atom of a skeleton of a “substituted aryl group” in the specific example group G1B below, and a group derived by further substituting a hydrogen atom of a substituent of the “substituted aryl group” in the specific example group G1B below.

### Unsubstituted Aryl Group (Specific Example Group G1A):

a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, benzanthryl group, phenanthryl group, benzophenanthryl group, phenalenyl group, pyrenyl group, chrysenyl group, benzochrysenyl group, triphenylenyl group, benzotriphenylenyl group, tetracenyl group, pentacenyl group, fluorenyl group, 9,9′-spirobifluorenyl group, benzofluorenyl group, dibenzofluorenyl group, fluoranthenyl group, benzofluoranthenyl group, perylenyl group, and monovalent aryl group derived by removing one hydrogen atom from cyclic structures represented by formulae (TEMP-1) to (TEMP-15) below.

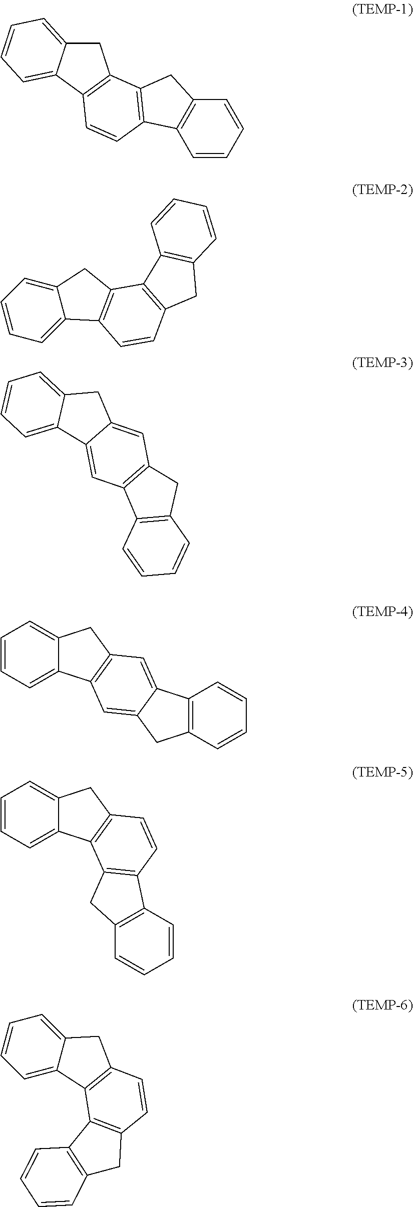

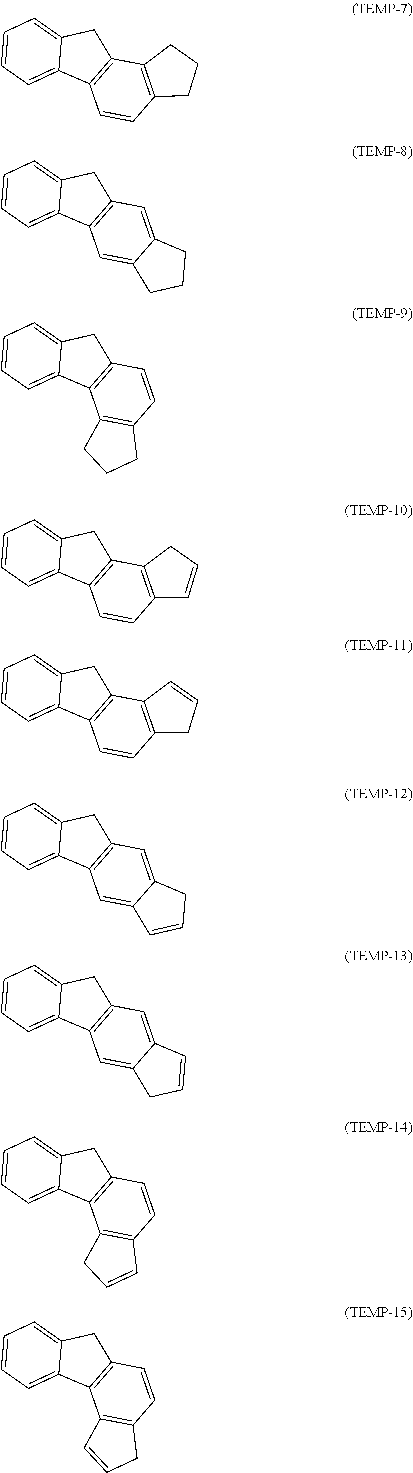

### Substituted Aryl Group (Specific Example Group G1B):

an o-tolyl group, m-tolyl group, p-tolyl group, para-xylyl group, meta-xylyl group, ortho-xylyl group, para-isopropylphenyl group, meta-isopropylphenyl group, ortho-isopropylphenyl group, para-t-butylphenyl group, meta-t-butylphenyl group, ortho-t-butylphenyl group, 3,4,5-trimethyl phenyl group, 9,9-dimethylfluorenyl group, 9,9-diphenylfluorenyl group, 9,9-bis(4-methylphenyl)fluorenyl group, 9,9-bis(4-isopropylphenyl)fluorenyl group, 9,9-bis(4-t-butylphenyl)fluorenyl group, cyanophenyl group, triphenylsilylphenyl group, trimethylsilylphenyl group, phenylnaphthyl group, naphthylphenyl group, and group derived by substituting at least one hydrogen atom of a monovalent group derived from one of the cyclic structures represented by the formulae (TEMP-1) to (TEMP-15) with a substituent.

### Substituted or Unsubstituted Heterocyclic Group

The “heterocyclic group” mentioned herein refers to a cyclic group having at least one hetero atom in the ring atoms.

Specific examples of the hetero atom include a nitrogen atom, oxygen atom, sulfur atom, silicon atom, phosphorus atom, and boron atom.

The “heterocyclic group” mentioned herein is a monocyclic group or a fused-ring group.

The “heterocyclic group” mentioned herein is an aromatic heterocyclic group or a non-aromatic heterocyclic group.

Specific examples (specific example group G2) of the “substituted or unsubstituted heterocyclic group” mentioned herein include unsubstituted heterocyclic groups (specific example group G2A) and substituted heterocyclic groups (specific example group G2B).

(Herein, an unsubstituted heterocyclic group refers to an “unsubstituted heterocyclic group” in a “substituted or unsubstituted heterocyclic group,” and a substituted heterocyclic group refers to a “substituted heterocyclic group” in a “substituted or unsubstituted heterocyclic group.”)

A simply termed “heterocyclic group” herein includes both of an “unsubstituted heterocyclic group” and a “substituted heterocyclic group.”

The “substituted heterocyclic group” refers to a group derived by substituting at least one hydrogen atom in an “unsubstituted heterocyclic group” with a substituent.

Specific examples of the “substituted heterocyclic group” include a group derived by substituting at least one hydrogen atom in the “unsubstituted heterocyclic group” in the specific example group G2A below with a substituent, and examples of the substituted heterocyclic group in the specific example group G2B below.

It should be noted that the examples of the “unsubstituted heterocyclic group” and the “substituted heterocyclic group” mentioned herein are merely exemplary, and the “substituted heterocyclic group” mentioned herein includes a group derived by further substituting a hydrogen atom bonded to a ring atom of a skeleton of a “substituted heterocyclic group” in the specific example group G2B below, and a group derived by further substituting a hydrogen atom of a substituent of the “substituted heterocyclic group” in the specific example group G2B below.

The specific example group G2A includes, for instance, unsubstituted heterocyclic groups including a nitrogen atom (specific example group G2A1) below, unsubstituted heterocyclic groups including an oxygen atom (specific example group G2A2) below, unsubstituted heterocyclic groups including a sulfur atom (specific example group G2A3) below, and monovalent heterocyclic groups (specific example group G2A4) derived by removing a hydrogen atom from cyclic structures represented by formulae (TEMP-16) to (TEMP-33) below.

The specific example group G2B includes, for instance, substituted heterocyclic groups including a nitrogen atom (specific example group G2B1) below, substituted heterocyclic groups including an oxygen atom (specific example group G2B2) below, substituted heterocyclic groups including a sulfur atom (specific example group G2B3) below, and groups derived by substituting at least one hydrogen atom of the monovalent heterocyclic groups (specific example group G2B4) derived from the cyclic structures represented by formulae (TEMP-16) to (TEMP-33) below.

### Unsubstituted Heterocyclic Groups Including Nitrogen Atom (Specific Example Group G2A1):

a pyrrolyl group, imidazolyl group, pyrazolyl group, triazolyl group, tetrazolyl group, oxazolyl group, isoxazolyl group, oxadiazolyl group, thiazolyl group, isothiazolyl group, thiadiazolyl group, pyridyl group, pyridazynyl group, pyrimidinyl group, pyrazinyl group, triazinyl group, indolyl group, isoindolyl group, indolizinyl group, quinolizinyl group, quinolyl group, isoquinolyl group, cinnolyl group, phthalazinyl group, quinazolinyl group, quinoxalinyl group, benzimidazolyl group, indazolyl group, phenanthrolinyl group, phenanthridinyl group, acridinyl group, phenazinyl group, carbazolyl group, benzocarbazolyl group, morpholino group, phenoxazinyl group, phenothiazinyl group, azacarbazolyl group, and diazacarbazolyl group.

### Unsubstituted Heterocyclic Groups Including Oxygen Atom (Specific Example Group G2A2):

a furyl group, oxazolyl group, isoxazolyl group, oxadiazolyl group, xanthenyl group, benzofuranyl group, isobenzofuranyl group, dibenzofuranyl group, naphthobenzofuranyl group, benzoxazolyl group, benzisoxazolyl group, phenoxazinyl group, morpholino group, dinaphthofuranyl group, azadibenzofuranyl group, diazadibenzofuranyl group, azanaphthobenzofuranyl group, and diazanaphthobenzofuranyl group.

### Unsubstituted Heterocyclic Groups Including Sulfur Atom (Specific Example Group G2A3):

a thienyl group, thiazolyl group, isothiazolyl group, thiadiazolyl group, benzothiophenyl group (benzothienyl group), isobenzothiophenyl group (isobenzothienyl group), dibenzothiophenyl group (dibenzothienyl group), naphthobenzothiophenyl group (nahthobenzothienyl group), benzothiazolyl group, benzisothiazolyl group, phenothiazinyl group, dinaphthothiophenyl group (dinaphthothienyl group), azadibenzothiophenyl group (azadibenzothienyl group), diazadibenzothiophenyl group (diazadibenzothienyl group), azanaphthobenzothiophenyl group (azanaphthobenzothienyl group), and diazanaphthobenzothiophenyl group (diazanaphthobenzothienyl group).
   Monovalent Heterocyclic Groups Derived by Removing One Hydrogen Atom from Cyclic Structures Represented by Formulae (TEMP-16) to (TEMP-33) (Specific Example Group G2A4):

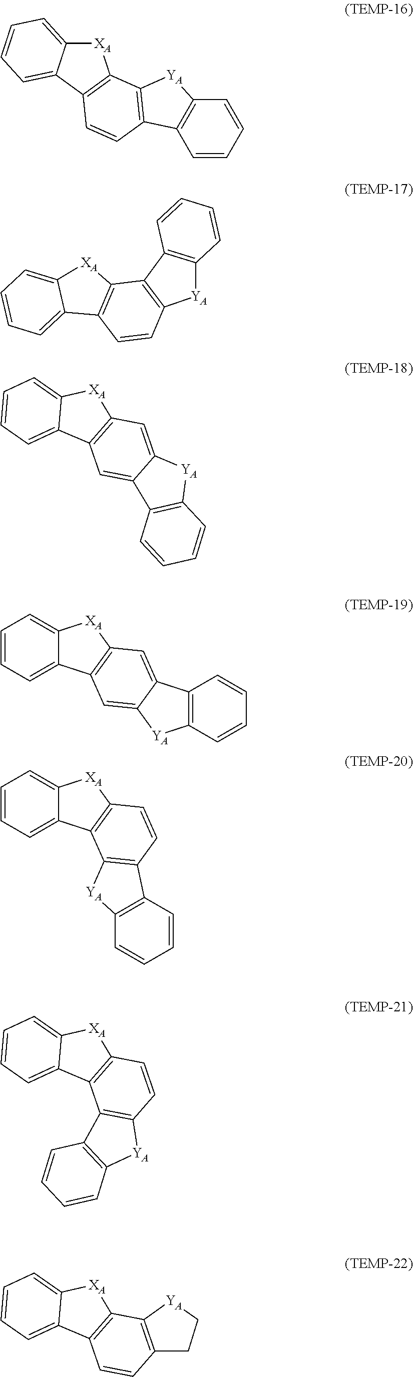

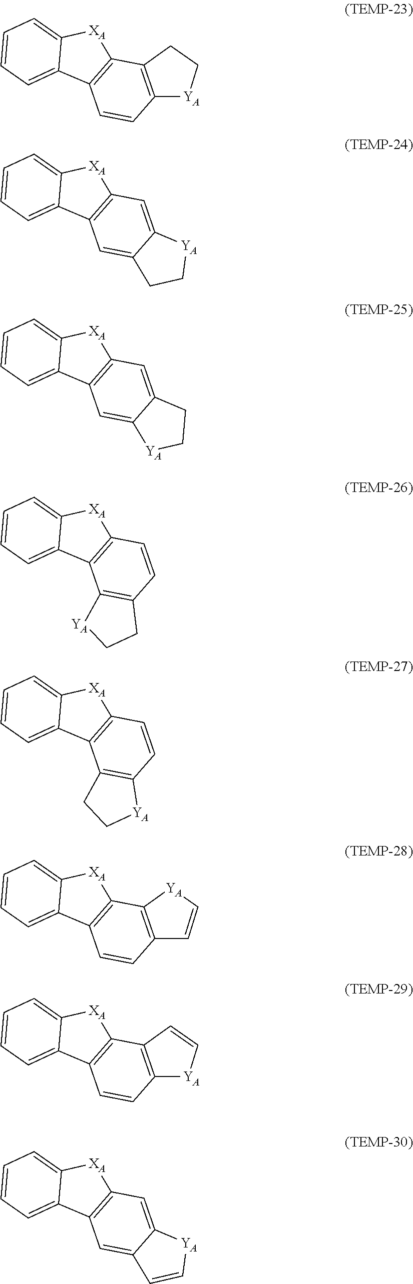

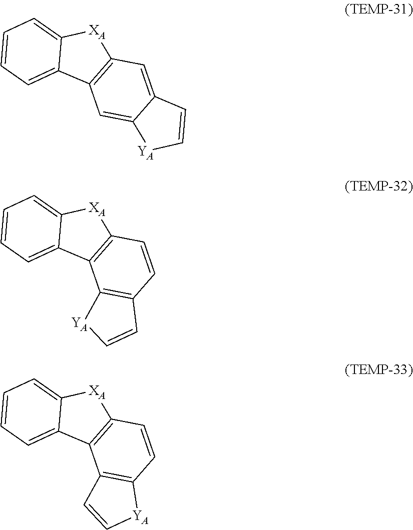

In the formulae (TEMP-16) to (TEMP-33), X_{A} and Y_{A} are each independently an oxygen atom, a sulfur atom, NH or CH₂, provided that at least one of X_{A} or Y_{A} is an oxygen atom, a sulfur atom, or NH.

When at least one of X_{A} or Y_{A} in the formulae (TEMP-16) to (TEMP-33) is NH or CH₂, the monovalent heterocyclic groups derived from the cyclic structures represented by the formulae (TEMP-16) to (TEMP-33) include a monovalent group derived by removing one hydrogen atom from NH or CH₂.

### Substituted Heterocyclic Groups Including Nitrogen Atom (Specific Example Group G2B1):

(9-phenyl)carbazolyl group, (9-biphenylyl)carbazolyl group, (9-phenyl)phenylcarbazolyl group, (9-naphthyl)carbazolyl group, diphenylcarbazole-9-yl group, phenylcarbazole-9-yl group, methylbenzimidazolyl group, ethylbenzimidazolyl group, phenyltriazinyl group, biphenylyltriazinyl group, diphenyltriazinyl group, phenylquinazolinyl group, and biphenylquinazolinyl group.

### Substituted Heterocyclic Groups Including Oxygen Atom (Specific Example Group G2B2):

phenyldibenzofuranyl group, methyldibenzofuranyl group, t-butyldibenzofuranyl group, and monovalent residue of spiro[9H-xanthene-9,9′-[9H]fluorene].

### Substituted Heterocyclic Groups Including Sulfur Atom (Specific Example Group G2B3):

a phenyldibenzothiophenyl group, methyldibenzothiophenyl group, t-butyldibenzothiophenyl group, and monovalent residue of spiro[9H-thioxanthene-9,9′-[9H]fluorene].
   Groups Obtained by Substituting at Least One Hydrogen Atom of Monovalent Heterocyclic Group Derived from Cyclic Structures Represented by Formulae (TEMP-16) to (TEMP-33) with Substituent (Specific Example Group G2B4):

The “at least one hydrogen atom of a monovalent heterocyclic group” means at least one hydrogen atom selected from a hydrogen atom bonded to a ring carbon atom of the monovalent heterocyclic group, a hydrogen atom bonded to a nitrogen atom of at least one of X^{A} or Y_{A} in a form of NH, and a hydrogen atom of one of X_{A} and Y_{A} in a form of a methylene group (CH₂).

### Substituted or Unsubstituted Alkyl Group

Specific examples (specific example group G3) of the “substituted or unsubstituted alkyl group” mentioned herein include unsubstituted alkyl groups (specific example group G3A) and substituted alkyl groups (specific example group G3B) below.

(Herein, an unsubstituted alkyl group refers to an “unsubstituted alkyl group” in a “substituted or unsubstituted alkyl group,” and a substituted alkyl group refers to a “substituted alkyl group” in a “substituted or unsubstituted alkyl group.”)

A simply termed “alkyl group” herein includes both of an “unsubstituted alkyl group” and a “substituted alkyl group”.

The “substituted alkyl group” refers to a group derived by substituting at least one hydrogen atom in an “unsubstituted alkyl group” with a substituent.

Specific examples of the “substituted alkyl group” include a group derived by substituting at least one hydrogen atom of an “unsubstituted alkyl group” (specific example group G3A) below with a substituent, and examples of the substituted alkyl group (specific example group G3B) below.

Herein, the alkyl group for the “unsubstituted alkyl group” refers to a chain alkyl group.

Accordingly, the “unsubstituted alkyl group” include linear “unsubstituted alkyl group” and branched “unsubstituted alkyl group.”

It should be noted that the examples of the “unsubstituted alkyl group” and the “substituted alkyl group” mentioned herein are merely exemplary, and the “substituted alkyl group” mentioned herein includes a group derived by further substituting a hydrogen atom of a skeleton of the “substituted alkyl group” in the specific example group G3B, and a group derived by further substituting a hydrogen atom of a substituent of the “substituted alkyl group” in the specific example group G3B.

### Unsubstituted Alkyl Group (Specific Example Group G3A):

a methyl group, ethyl group, n-propyl group, isopropyl group, n-butyl group, isobutyl group, s-butyl group, and t-butyl group.

### Substituted Alkyl Group (Specific Example Group G3B):

a heptafluoropropyl group (including isomer thereof), pentafluoroethyl group, 2,2,2-trifluoroethyl group, and trifluoromethyl group.

### Substituted or Unsubstituted Alkenyl Group

Specific examples (specific example group G4) of the “substituted or unsubstituted alkenyl group” mentioned herein include unsubstituted alkenyl groups (specific example group G4A) and substituted alkenyl groups (specific example group G4B).

(Herein, an unsubstituted alkenyl group refers to an “unsubstituted alkenyl group” in a “substituted or unsubstituted alkenyl group,” and a substituted alkenyl group refers to a “substituted alkenyl group” in a “substituted or unsubstituted alkenyl group.”)

A simply termed “alkenyl group” herein includes both of an “unsubstituted alkenyl group” and a “substituted alkenyl group”.

The “substituted alkenyl group” refers to a group derived by substituting at least one hydrogen atom in an “unsubstituted alkenyl group” with a substituent.

Specific examples of the “substituted alkenyl group” include an “unsubstituted alkenyl group” (specific example group G4A) substituted by a substituent, and examples of the substituted alkenyl group (specific example group G4B) below.

It should be noted that the examples of the “unsubstituted alkenyl group” and the “substituted alkenyl group” mentioned herein are merely exemplary, and the “substituted alkenyl group” mentioned herein includes a group derived by further substituting a hydrogen atom of a skeleton of the “substituted alkenyl group” in the specific example group G4B with a substituent, and a group derived by further substituting a hydrogen atom of a substituent of the “substituted alkenyl group” in the specific example group G4B with a substituent.

### Unsubstituted Alkenyl Group (Specific Example Group G4A):

a vinyl group, allyl group, 1-butenyl group, 2-butenyl group, and 3-butenyl group.

### Substituted Alkenyl Group (Specific Example Group G4B):

a 1,3-butanedienyl group, 1-methylvinyl group, 1-methylallyl group, 1,1-dimethylallyl group, 2-methylallyl group, and 1,2-dimethylallyl group.

### Substituted or Unsubstituted Alkynyl Group

Specific examples (specific example group G5) of the “substituted or unsubstituted alkynyl group” mentioned herein include unsubstituted alkynyl groups (specific example group G5A) below.

(Herein, an unsubstituted alkynyl group refers to an “unsubstituted alkynyl group” in a “substituted or unsubstituted alkynyl group.”)

A simply termed “alkynyl group” herein includes both of “unsubstituted alkynyl group” and “substituted alkynyl group”.

The “substituted alkynyl group” refers to a group derived by substituting at least one hydrogen atom in an “unsubstituted alkynyl group” with a substituent.

Specific examples of the “substituted alkynyl group” include a group derived by substituting at least one hydrogen atom of the “unsubstituted alkynyl group” (specific example group G5A) below with a substituent.

### Unsubstituted Alkynyl Group (Specific Example Group G5A):

an ethynyl group.

### Substituted or Unsubstituted Cycloalkyl Group

Specific examples (specific example group G6) of the “substituted or unsubstituted cycloalkyl group” mentioned herein include unsubstituted cycloalkyl groups (specific example group G6A) and substituted cycloalkyl groups (specific example group G6B).

(Herein, an unsubstituted cycloalkyl group refers to an “unsubstituted cycloalkyl group” in a “substituted or unsubstituted cycloalkyl group,” and a substituted cycloalkyl group refers to a “substituted cycloalkyl group” in a “substituted or unsubstituted cycloalkyl group.”)

A simply termed “cycloalkyl group” herein includes both of “unsubstituted cycloalkyl group” and “substituted cycloalkyl group”.

The “substituted cycloalkyl group” refers to a group derived by substituting at least one hydrogen atom of an “unsubstituted cycloalkyl group” with a substituent.

Specific examples of the “substituted cycloalkyl group” include a group derived by substituting at least one hydrogen atom of the “unsubstituted cycloalkyl group” (specific example group G6A) below with a substituent, and examples of the substituted cycloalkyl group (specific example group G6B) below.

It should be noted that the examples of the “unsubstituted cycloalkyl group” and the “substituted cycloalkyl group” mentioned herein are merely exemplary, and the “substituted cycloalkyl group” mentioned herein includes a group derived by substituting at least one hydrogen atom bonded to a carbon atom of a skeleton of the “substituted cycloalkyl group” in the specific example group G6B with a substituent, and a group derived by further substituting a hydrogen atom of a substituent of the “substituted cycloalkyl group” in the specific example group G6B with a substituent.

### Unsubstituted Cycloalkyl Group (Specific Example Group G6A):

a cyclopropyl group, cyclobutyl group, cyclopentyl group, cyclohexyl group, 1-adamantyl group, 2-adamantyl group, 1-norbornyl group, and 2-norbornyl group.

### Substituted Cycloalkyl Group (Specific Example Group G6B):

a 4-methylcyclohexyl group.

Group Represented by —Si(R₉₀₁)(R₉₀₂)(R₉₀₃)

Specific examples (specific example group G7) of the group represented herein by —Si(R₉₀₁)(R₉₀₂)(R₉₀₃) include: —Si(G1)(G1)(G1); —Si(G1)(G2)(G2); —Si(G1)(G1)(G2); —Si(G2)(G2)(G2); —Si(G3)(G3)(G3); and —Si(G6)(G6)(G6);
where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3;
G6 represents a “substituted or unsubstituted cycloalkyl group” in the specific example group G6;
a plurality of G1 in —Si(G1)(G1)(G1) are mutually the same or different;
a plurality of G2 in —Si(G1)(G2)(G2) are mutually the same or different;
a plurality of G1 in —Si(G1)(G1)(G2) are mutually the same or different;
a plurality of G2 in —Si(G2)(G2)(G2) are mutually the same or different;
a plurality of G3 in —Si(G3)(G3)(G3) are mutually the same or different; and
a plurality of G6 in —Si(G6)(G6)(G6) are mutually the same or different.

### Group Represented by —O—(R₉₀₄)

Specific examples (specific example group G8) of a group represented by —O—(R₉₀₄) herein include: —O(G1); —O(G2); —O(G3); and —O(G6);
where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3; and
G6 represents a “substituted or unsubstituted cycloalkyl group” in the specific example group G6.

### Group Represented by —S—(R₉₀₅)

Specific examples (specific example group G9) of a group represented herein by —S—(R₉₀₅) include: —S(G1); —S(G2); —S(G3); and —S(G6);

where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3; and
G6 represents a “substituted or unsubstituted cycloalkyl group” in the specific example group G6.
   Group Represented by —N(R₉₀₆)(R₉₀₇)

Specific examples (specific example group G10) of a group represented herein by —N(R₉₀₆)(R₉₀₇) include: —N(G1)(G1); —N(G2)(G2); —N(G1)(G2); —N(G3)(G3); and —N(G6)(G6),

where:
G1 represents a “substituted or unsubstituted aryl group” in the specific example group G1;
G2 represents a “substituted or unsubstituted heterocyclic group” in the specific example group G2;
G3 represents a “substituted or unsubstituted alkyl group” in the specific example group G3;
G6 represents a “substituted or unsubstituted cycloalkyl group” in the specific example group G6;
a plurality of G1 in —N(G1)(G1) are mutually the same or different;
a plurality of G2 in —N(G2)(G2) are mutually the same or different;
a plurality of G3 in —N(G3)(G3) are mutually the same or different; and
a plurality of G6 in —N(G6)(G6) are mutually the same or different.

### Halogen Atom

Specific examples (specific example group G11) of “halogen atom” mentioned herein include a fluorine atom, chlorine atom, bromine atom, and iodine atom.

### Substituted or Unsubstituted Fluoroalkyl Group

The “substituted or unsubstituted fluoroalkyl group” mentioned herein refers to a group derived by substituting at least one hydrogen atom bonded to at least one of carbon atoms forming an alkyl group in the “substituted or unsubstituted alkyl group” with a fluorine atom, and also includes a group (perfluoro group) derived by substituting all of hydrogen atoms bonded to carbon atoms forming the alkyl group in the “substituted or unsubstituted alkyl group” with fluorine atoms.

An “unsubstituted fluoroalkyl group” has, unless otherwise specified herein, 1 to 50, preferably 1 to 30, more preferably 1 to 18 carbon atoms.

The “substituted fluoroalkyl group” refers to a group derived by substituting at least one hydrogen atom in a “fluoroalkyl group” with a substituent.

It should be noted that the examples of the “substituted fluoroalkyl group” mentioned herein include a group derived by further substituting at least one hydrogen atom bonded to a carbon atom of an alkyl chain of a “substituted fluoroalkyl group” with a substituent, and a group derived by further substituting at least one hydrogen atom of a substituent of the “substituted fluoroalkyl group” with a substituent.

Specific examples of the “unsubstituted fluoroalkyl group” include a group derived by substituting at least one hydrogen atom of the “alkyl group” (specific example group G3) with a fluorine atom.

### Substituted or Unsubstituted Haloalkyl Group

The “substituted or unsubstituted haloalkyl group” mentioned herein refers to a group derived by substituting at least one hydrogen atom bonded to carbon atoms forming the alkyl group in the “substituted or unsubstituted alkyl group” with a halogen atom, and also includes a group derived by substituting all hydrogen atoms bonded to carbon atoms forming the alkyl group in the “substituted or unsubstituted alkyl group” with halogen atoms.

An “unsubstituted haloalkyl group” has, unless otherwise specified herein, 1 to 50, preferably 1 to 30, and more preferably 1 to 18 carbon atoms.

The “substituted haloalkyl group” refers to a group derived by substituting at least one hydrogen atom in a “haloalkyl group” with a substituent.

It should be noted that the examples of the “substituted haloalkyl group” mentioned herein include a group derived by further substituting at least one hydrogen atom bonded to a carbon atom of an alkyl chain of a “substituted haloalkyl group” with a substituent, and a group derived by further substituting at least one hydrogen atom of a substituent of the “substituted haloalkyl group” with a substituent.

Specific examples of the “unsubstituted haloalkyl group” include a group derived by substituting at least one hydrogen atom of the “alkyl group” (specific example group G3) with a halogen atom.

The haloalkyl group is sometimes referred to as a halogenated alkyl group.

### Substituted or Unsubstituted Alkoxy Group

Specific examples of a “substituted or unsubstituted alkoxy group” mentioned herein include a group represented by —O(G3), G3 being the “substituted or unsubstituted alkyl group” in the specific example group G3.

An “unsubstituted alkoxy group” has, unless otherwise specified herein, 1 to 50, preferably 1 to 30, more preferably 1 to 18 carbon atoms.

### Substituted or Unsubstituted Alkylthio Group

Specific examples of a “substituted or unsubstituted alkylthio group” mentioned herein include a group represented by —S(G3), G3 being the “substituted or unsubstituted alkyl group” in the specific example group G3.

An “unsubstituted alkylthio group” has, unless otherwise specified herein, 1 to 50, preferably 1 to 30, more preferably 1 to 18 carbon atoms.

### Substituted or Unsubstituted Aryloxy Group

Specific examples of a “substituted or unsubstituted aryloxy group” mentioned herein include a group represented by —O(G1), G1 being the “substituted or unsubstituted aryl group” in the specific example group G1.

An “unsubstituted aryloxy group” has, unless otherwise specified herein, 6 to 50, preferably 6 to 30, more preferably 6 to 18 ring carbon atoms.

### Substituted or Unsubstituted Arylthio Group

Specific examples of a “substituted or unsubstituted arylthio group” mentioned herein include a group represented by —S(G1), G1 being the “substituted or unsubstituted aryl group” in the specific example group G1.

An “unsubstituted arylthio group” has, unless otherwise specified herein, 6 to 50, preferably 6 to 30, more preferably 6 to 18 ring carbon atoms.

Substituted or Unsubstituted Trialkylsilyl Group Specific examples of a “trialkylsilyl group” mentioned herein include a group represented by —Si(G3)(G3)(G3), G3 being the “substituted or unsubstituted alkyl group” in the specific example group G3.

The plurality of G3 in —Si(G3)(G3)(G3) are mutually the same or different.

Each of the alkyl groups in the “trialkylsilyl group” has, unless otherwise specified herein, 1 to 50, preferably 1 to 20, more preferably 1 to 6 carbon atoms.

### Substituted or Unsubstituted Aralkyl Group

Specific examples of a “substituted or unsubstituted aralkyl group” mentioned herein include a group represented by -(G3)-(G1), G3 being the “substituted or unsubstituted alkyl group” in the specific example group G3, G1 being the “substituted or unsubstituted aryl group” in the specific example group G1.

Accordingly, the “aralkyl group” is a group derived by substituting a hydrogen atom of the “alkyl group” with a substituent in a form of the “aryl group,” which is an example of the “substituted alkyl group.”

An “unsubstituted aralkyl group,” which is an “unsubstituted alkyl group” substituted by an “unsubstituted aryl group,” has, unless otherwise specified herein, 7 to 50 carbon atoms, preferably 7 to 30 carbon atoms, more preferably 7 to 18 carbon atoms.

Specific examples of the “substituted or unsubstituted aralkyl group” include a benzyl group, 1-phenylethyl group, 2-phenylethyl group, 1-phenylisopropyl group, 2-phenylisopropyl group, phenyl-t-butyl group, α-naphthylmethyl group, 1-α-naphthylethyl group, 2-α-naphthylethyl group, 1-α-naphthylisopropyl group, 2-α-naphthylisopropyl group, β-naphthylmethyl group, 1-β-naphthylethyl group, 2-β-naphthylethyl group, 1-β-naphthylisopropyl group, and 2-β-naphthylisopropyl group.

Preferable examples of the substituted or unsubstituted aryl group mentioned herein include, unless otherwise specified herein, a phenyl group, p-biphenyl group, m-biphenyl group, o-biphenyl group, p-terphenyl-4-yl group, p-terphenyl-3-yl group, p-terphenyl-2-yl group, m-terphenyl-4-yl group, m-terphenyl-3-yl group, m-terphenyl-2-yl group, o-terphenyl-4-yl group, o-terphenyl-3-yl group, o-terphenyl-2-yl group, 1-naphthyl group, 2-naphthyl group, anthryl group, phenanthryl group, pyrenyl group, chrysenyl group, triphenylenyl group, fluorenyl group, 9,9′-spirobifluorenyl group, 9,9-dimethylfluorenyl group, and 9,9-diphenylfluorenyl group.

Preferable examples of the substituted or unsubstituted heterocyclic group mentioned herein include, unless otherwise specified herein, a pyridyl group, pyrimidinyl group, triazinyl group, quinolyl group, isoquinolyl group, quinazolinyl group, benzimidazolyl group, phenanthrolinyl group, carbazolyl group (1-carbazolyl group, 2-carbazolyl group, 3-carbazolyl group, 4-carbazolyl group, or 9-carbazolyl group), benzocarbazolyl group, azacarbazolyl group, diazacarbazolyl group, dibenzofuranyl group, naphthobenzofuranyl group, azadibenzofuranyl group, diazadibenzofuranyl group, dibenzothiophenyl group, naphthobenzothiophenyl group, azadibenzothiophenyl group, diazadibenzothiophenyl group, (9-phenyl)carbazolyl group ((9-phenyl)carbazole-1-yl group, (9-phenyl)carbazole-2-yl group, (9-phenyl)carbazole-3-yl group, or (9-phenyl)carbazole-4-yl group), (9-biphenylyl)carbazolyl group, (9-phenyl)phenylcarbazolyl group, diphenylcarbazole-9-yl group, phenylcarbazole-9-yl group, phenyltriazinyl group, biphenylyltriazinyl group, diphenyltriazinyl group, phenyldibenzofuranyl group, and phenyldibenzothiophenyl group.

The carbazolyl group mentioned herein is, unless otherwise specified herein, specifically a group represented by one of formulae below.

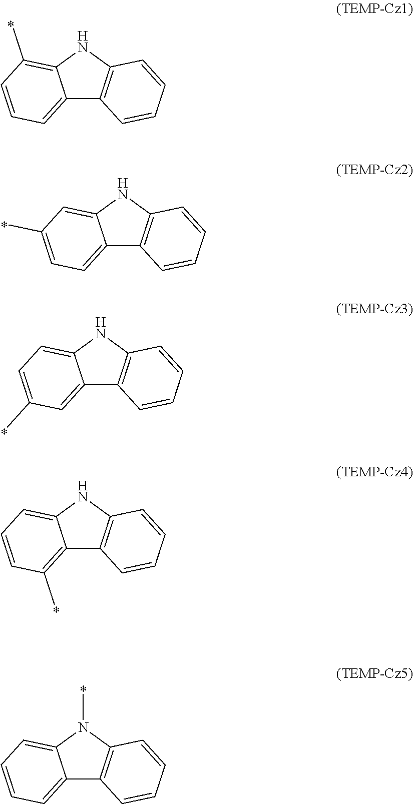

The (9-phenyl)carbazolyl group mentioned herein is, unless otherwise specified herein, specifically a group represented by one of formulae below.

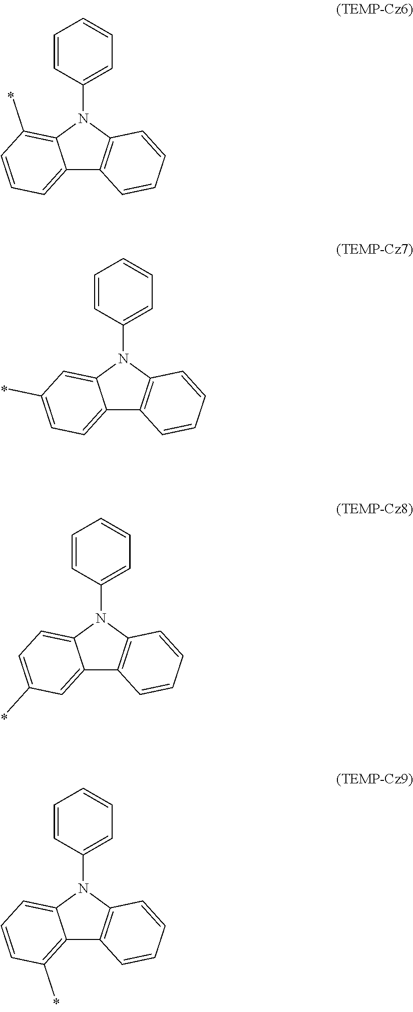 [SMILES: Cc1cccc2c3ccccc3n(-c3ccccc3)c12 ; Cc1ccc2c3ccccc3n(-c3ccccc3)c2c1 ; Cc1ccc2c(c1)c1ccccc1n2-c1ccccc1 ; Cc1cccc2c1c1ccccc1n2-c1ccccc1]

In the formulae (TEMP-Cz1) to (TEMP-Cz9), * represents a bonding position.

The dibenzofuranyl group and dibenzothiophenyl group mentioned herein are, unless otherwise specified herein, each specifically represented by one of formulae below.

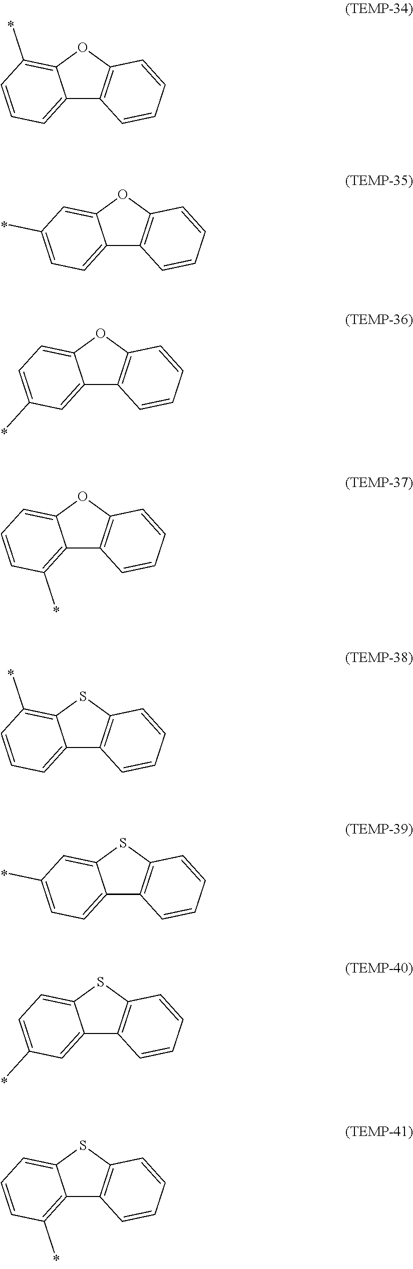

In the formulae (TEMP-34) to (TEMP-41), * represents a bonding position.

Preferable examples of the substituted or unsubstituted alkyl group mentioned herein include, unless otherwise specified herein, a methyl group, ethyl group, propyl group, isopropyl group, n-butyl group, isobutyl group, and t-butyl group.

### Substituted or Unsubstituted Arylene Group

The “substituted or unsubstituted arylene group” mentioned herein is, unless otherwise specified herein, a divalent group derived by removing one hydrogen atom on an aryl ring of the “substituted or unsubstituted aryl group.”

Specific examples of the “substituted or unsubstituted arylene group” (specific example group G12) include a divalent group derived by removing one hydrogen atom on an aryl ring of the “substituted or unsubstituted aryl group” in the specific example group G1.

### Substituted or Unsubstituted Divalent Heterocyclic Group

The “substituted or unsubstituted divalent heterocyclic group” mentioned herein is, unless otherwise specified herein, a divalent group derived by removing one hydrogen atom on a heterocycle of the “substituted or unsubstituted heterocyclic group.”

Specific examples of the “substituted or unsubstituted divalent heterocyclic group” (specific example group G13) include a divalent group derived by removing one hydrogen atom on a heterocyclic ring of the “substituted or unsubstituted heterocyclic group” in the specific example group G2.

### Substituted or Unsubstituted Alkylene Group

The “substituted or unsubstituted alkylene group” mentioned herein is, unless otherwise specified herein, a divalent group derived by removing one hydrogen atom on an alkyl chain of the “substituted or unsubstituted alkyl group.”

Specific examples of the “substituted or unsubstituted alkylene group” (specific example group G14) include a divalent group derived by removing one hydrogen atom on an alkyl chain of the “substituted or unsubstituted alkyl group” in the specific example group G3.

The substituted or unsubstituted arylene group mentioned herein is, unless otherwise specified herein, preferably any one of groups represented by formulae (TEMP-42) to (TEMP-68) below.

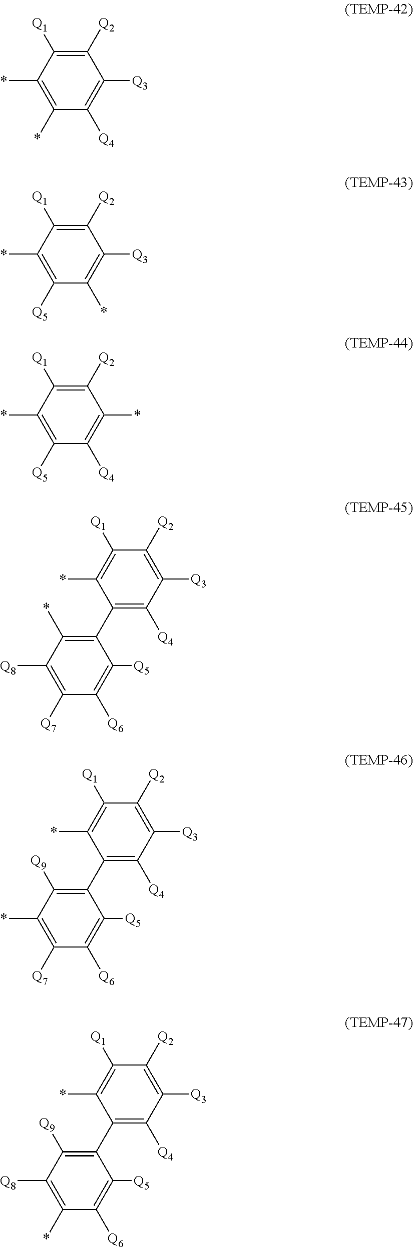

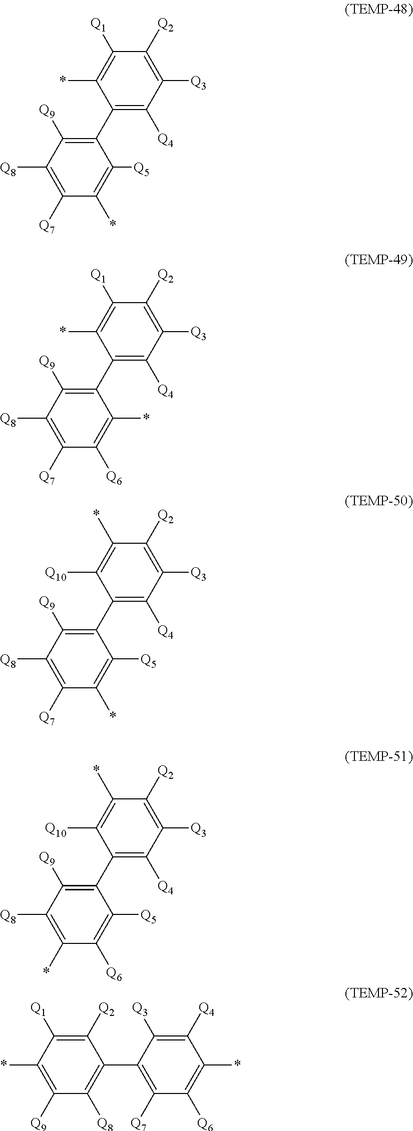

In the formulae (TEMP-42) to (TEMP-52), Q₁ to Q₁₀ are each independently a hydrogen atom or a substituent.

In the formulae (TEMP-42) to (TEMP-52), * represents a bonding position.

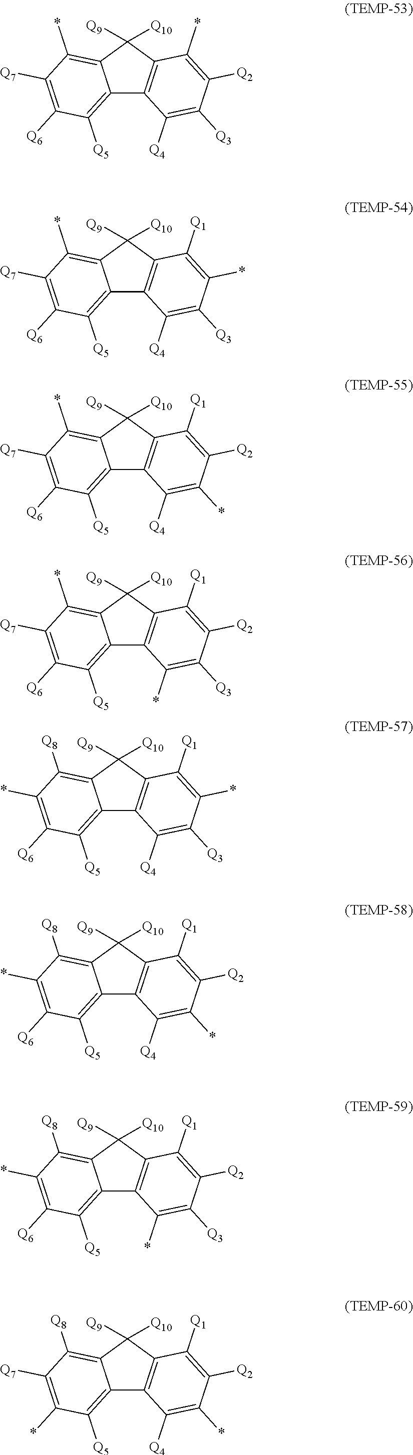

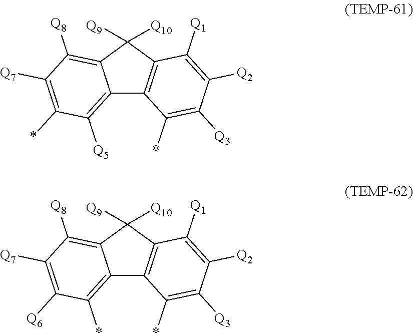

In the formulae (TEMP-53) to (TEMP-62), Q₁ to Q₁₀ are each independently a hydrogen atom or a substituent.

In the formulae, Q₉ and Q₁₀ may be mutually bonded through a single bond to form a ring.

In the formulae (TEMP-53) to (TEMP-62), * represents a bonding position.

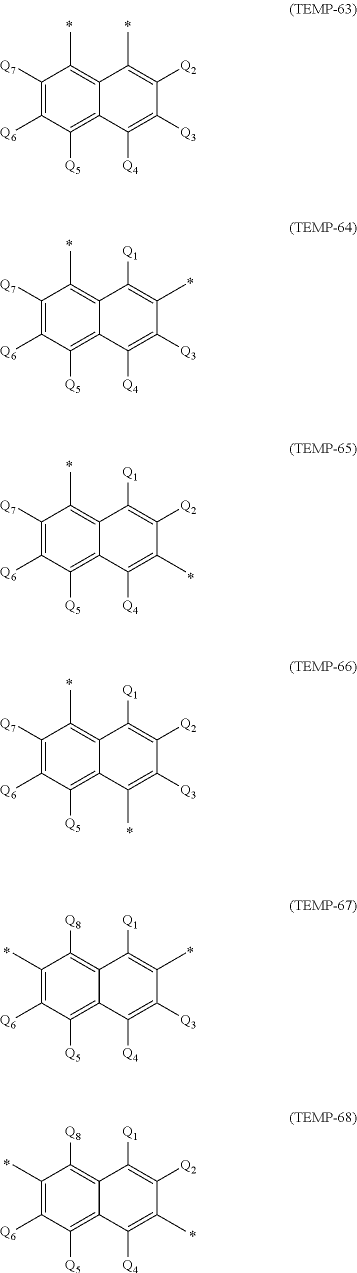

In the formulae (TEMP-63) to (TEMP-68), Q₁ to Q₈ are each independently a hydrogen atom or a substituent.

In the formulae (TEMP-63) to (TEMP-68), * represents a bonding position.

The substituted or unsubstituted divalent heterocyclic group mentioned herein is, unless otherwise specified herein, preferably a group represented by any one of formulae (TEMP-69) to (TEMP-102) below.

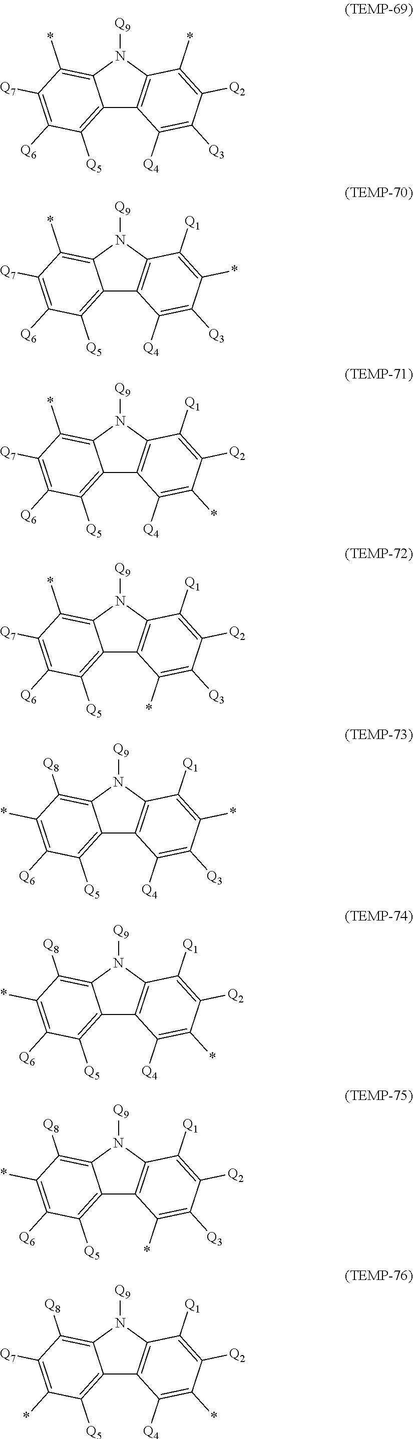

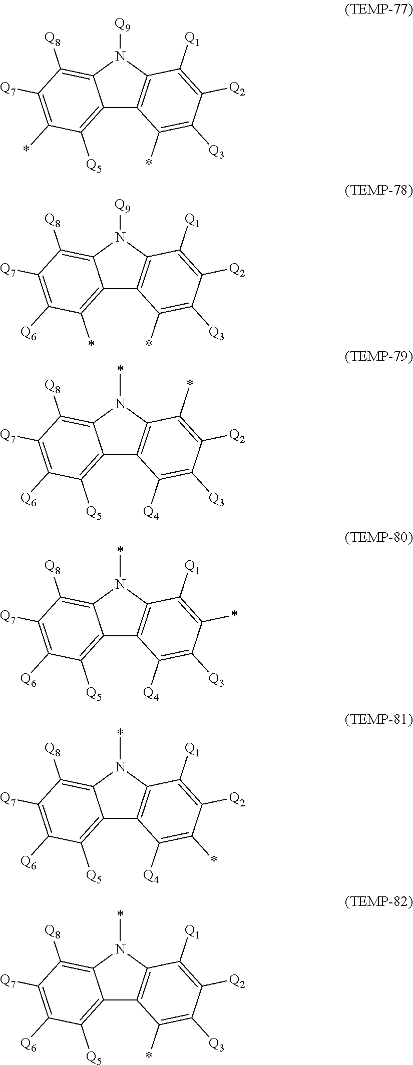

In the formulae (TEMP-69) to (TEMP-82), Q₁ to Q₉ are each independently a hydrogen atom or a substituent.

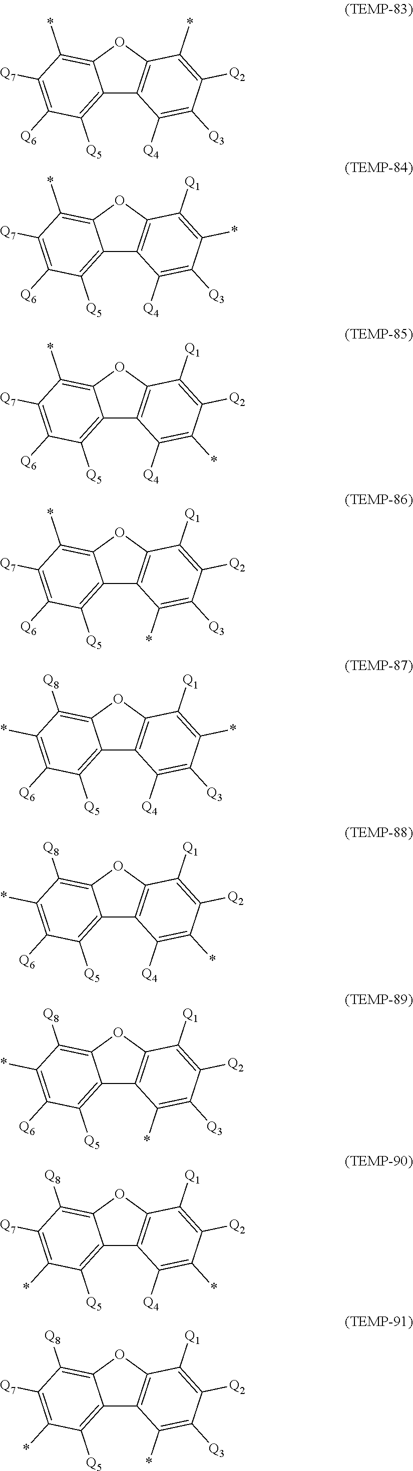

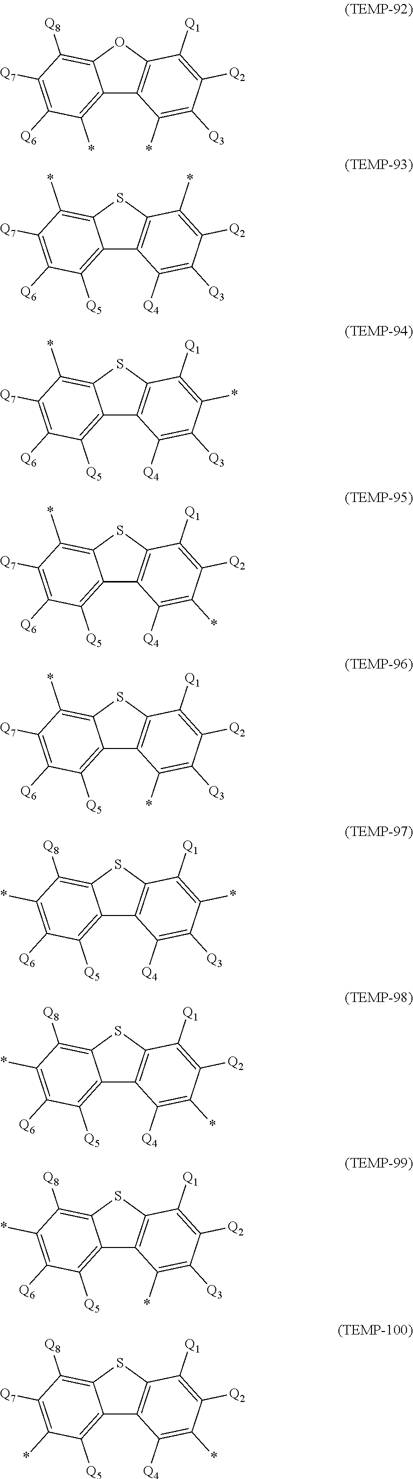

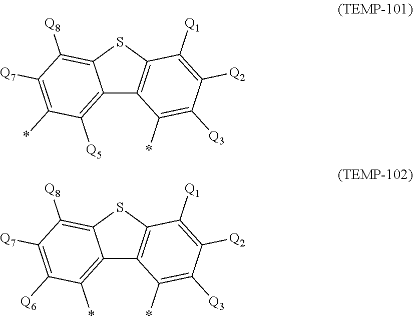

In the formulae (TEMP-83) to (TEMP-102), Q₁ to Q₈ are each independently a hydrogen atom or a substituent.

The substituent mentioned herein has been described above.

### Instance of “Bonded to Form Ring”

Instances where “at least one combination of adjacent two or more (of . . . ) are mutually bonded to form a substituted or unsubstituted monocyclic ring, mutually bonded to form a substituted or unsubstituted fused ring, or not mutually bonded” mentioned herein refer to instances where “at least one combination of adjacent two or more (of . . . ) are mutually bonded to form a substituted or unsubstituted monocyclic ring, “at least one combination of adjacent two or more (of . . . ) are mutually bonded to form a substituted or unsubstituted fused ring,” and “at least one combination of adjacent two or more (of . . . ) are not mutually bonded.”

Instances where “at least one combination of adjacent two or more (of . . . ) are mutually bonded to form a substituted or unsubstituted monocyclic ring” and where “at least one combination of adjacent two or more (of . . . ) are mutually bonded to form a substituted or unsubstituted fused ring” mentioned herein (these instances will be sometimes collectively referred to as an instance of “bonded to form a ring” hereinafter)

will be described below.

An anthracene compound having a basic skeleton in a form of an anthracene ring and represented by a formula (TEMP-103) below will be used as an example for the description.

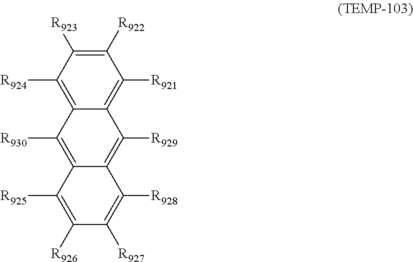

For instance, when “at least one combination of adjacent two or more of R₉₂₁ to R₉₃₀ are mutually bonded to form a ring,” the combination of adjacent ones of R₉₂₁ to R₉₃₀ (i.e. the combination at issue) is a combination of R₉₂₁ and R₉₂₂, a combination of R₉₂₂ and R₉₂₃, a combination of R₉₂₃ and R₉₂₄, a combination of R₉₂₄ and R₉₃₀, a combination of R₉₃₀ and R₉₂₅, a combination of R₉₂₅ and R₉₂₆, a combination of R₉₂₆ and R₉₂₇, a combination of R₉₂₇ and R₉₂₈, a combination of R₉₂₈ and R₉₂₉, or a combination of R₉₂₉ and R₉₂₁.

The term “at least one combination” means that two or more of the above combinations of adjacent two or more of R₉₂₁ to R₉₃₀ may simultaneously form rings.

For instance, when R₉₂₁ and R₉₂₂ are mutually bonded to form a ring Q_{A} and R₉₂₅ and R₉₂₆ are simultaneously mutually bonded to form a ring Q_{B}, the anthracene compound represented by the formula (TEMP-103) is represented by a formula (TEMP-104) below.

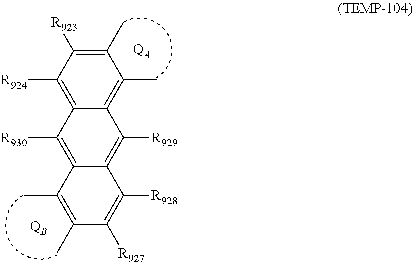

The instance where the “combination of adjacent two or more” form a ring means not only an instance where the “two” adjacent components are bonded but also an instance where adjacent “three or more” are bonded.

For instance, R₉₂₁ and R₉₂₂ are mutually bonded to form a ring Q_{A} and R₉₂₂ and R₉₂₃ are mutually bonded to form a ring Q_{C}, and mutually adjacent three components (R₉₂₁, R₉₂₂ and R₉₂₃) are mutually bonded to form a ring fused to the anthracene basic skeleton. In this case, the anthracene compound represented by the formula (TEMP-103) is represented by a formula (TEMP-105) below.

In the formula (TEMP-105) below, the ring Q_{A} and the ring Q_{C} share R₉₂₂.

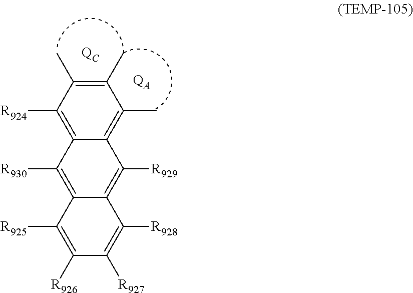

The formed “monocyclic ring” or “fused ring” may be, in terms of the formed ring in itself, a saturated ring or an unsaturated ring.

When the “combination of adjacent two” form a “monocyclic ring” or a “fused ring,” the “monocyclic ring” or “fused ring” may be a saturated ring or an unsaturated ring.

For instance, the ring Q_{A} and the ring Q_{B} formed in the formula (TEMP-104) are each independently a “monocyclic ring” or a “fused ring.”

Further, the ring Q_{A} and the ring Q_{C} formed in the formula (TEMP-105) are each a “fused ring.”

The ring Q_{A} and the ring Q_{C} in the formula (TEMP-105) are fused to form a fused ring.

When the ring Q_{A} in the formula (TEMP-104) is a benzene ring, the ring Q_{A} is a monocyclic ring.

When the ring Q_{A} in the formula (TEMP-104) is a naphthalene ring, the ring Q_{A} is a fused ring.

The “unsaturated ring” represents an aromatic hydrocarbon ring or an aromatic heterocycle.

The “saturated ring” represents an aliphatic hydrocarbon ring or a non-aromatic heterocycle.

Specific examples of the aromatic hydrocarbon ring include a ring formed by terminating a bond of a group in the specific example of the specific example group G1 with a hydrogen atom.

Specific examples of the aromatic heterocycle include a ring formed by terminating a bond of an aromatic heterocyclic group in the specific example of the specific example group G2 with a hydrogen atom.

Specific examples of the aliphatic hydrocarbon ring include a ring formed by terminating a bond of a group in the specific example of the specific example group G6 with a hydrogen atom.

The phrase “to form a ring” herein means that a ring is formed only by a plurality of atoms of a basic skeleton, or by a combination of a plurality of atoms of the basic skeleton and one or more optional atoms.

For instance, the ring Q_{A} formed by mutually bonding R₉₂₁ and R₉₂₂ shown in the formula (TEMP-104) is a ring formed by a carbon atom of the anthracene skeleton bonded to R₉₂₁, a carbon atom of the anthracene skeleton bonded to R₉₂₂, and one or more optional atoms.

Specifically, when the ring Q_{A} is a monocyclic unsaturated ring formed by R₉₂₁ and R₉₂₂, the ring formed by a carbon atom of the anthracene skeleton bonded to R₉₂₁, a carbon atom of the anthracene skeleton bonded to R₉₂₂, and four carbon atoms is a benzene ring.

The “optional atom” is, unless otherwise specified herein, preferably at least one atom selected from the group consisting of a carbon atom, nitrogen atom, oxygen atom, and sulfur atom.

A bond of the optional atom (e.g. a carbon atom and a nitrogen atom) not forming a ring may be terminated by a hydrogen atom or the like or may be substituted by an “optional substituent” described later.

When the ring includes an optional element other than carbon atom, the resultant ring is a heterocycle.

The number of “one or more optional atoms” forming the monocyclic ring or fused ring is, unless otherwise specified herein, preferably in a range from 2 to 15, more preferably in a range from 3 to 12, further preferably in a range from 3 to 5.

Unless otherwise specified herein, the ring, which may be a “monocyclic ring” or “fused ring,” is preferably a “monocyclic ring.”

Unless otherwise specified herein, the ring, which may be a “saturated ring” or “unsaturated ring,” is preferably an “unsaturated ring.”

Unless otherwise specified herein, the “monocyclic ring” is preferably a benzene ring.

Unless otherwise specified herein, the “unsaturated ring” is preferably a benzene ring.

When “at least one combination of adjacent two or more” (of . . . ) are “mutually bonded to form a substituted or unsubstituted monocyclic ring” or “mutually bonded to form a substituted or unsubstituted fused ring,” unless otherwise specified herein, at least one combination of adjacent two or more of components are preferably mutually bonded to form a substituted or unsubstituted “unsaturated ring” formed of a plurality of atoms of the basic skeleton, and 1 to 15 atoms of at least one element selected from the group consisting of carbon, nitrogen, oxygen and sulfur.

When the “monocyclic ring” or the “fused ring” has a substituent, the substituent is the substituent described in later-described “optional substituent.”

When the “monocyclic ring” or the “fused ring” has a substituent, specific examples of the substituent are the substituents described in the above under the subtitle “Substituent Mentioned Herein.”

When the “saturated ring” or the “unsaturated ring” has a substituent, the substituent is the substituent described in later-described “optional substituent.”

When the “monocyclic ring” or the “fused ring” has a substituent, specific examples of the substituent are the substituents described in the above under the subtitle “Substituent Mentioned Herein.”

The above is the description for the instances where “at least one combination of adjacent two or more (of . . . ) are mutually bonded to form a substituted or unsubstituted monocyclic ring” and “at least one combination of adjacent two or more (of . . . ) are mutually bonded to form a substituted or unsubstituted fused ring” mentioned herein (sometimes referred to as an instance of “bonded to form a ring”).

### Substituent for Substituted or Unsubstituted Group

In an exemplary embodiment herein, the substituent for the substituted or unsubstituted group (sometimes referred to as an “optional substituent” hereinafter) is, for instance, a group selected from the group consisting of an unsubstituted alkyl group having 1 to 50 carbon atoms, an unsubstituted alkenyl group having 2 to 50 carbon atoms, an unsubstituted alkynyl group having 2 to 50 carbon atoms, an unsubstituted cycloalkyl group having 3 to 50 ring carbon atoms, —Si(R₉₀₁)(R₉₀₂)(R₉₀₃), —O—(R₉₀₄), —S—(R₉₀₅), —N(R₉₀₆)(R₉₀₇), a halogen atom, a cyano group, a nitro group, an unsubstituted aryl group having 6 to 50 ring carbon atoms, and an unsubstituted heterocyclic group having 5 to 50 ring atoms;

R₉₀₁ to R₉₀₇ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when two or more R₉₀₁ are present, the two or more R₉₀₁ are mutually the same or different;
when two or more R₉₀₂ are present, the two or more R₉₀₂ are mutually the same or different;
when two or more R₉₀₃ are present, the two or more R₉₀₃ are mutually the same or different;
when two or more R₉₀₄ are present, the two or more R₉₀₄ are mutually the same or different;
when two or more R₉₀₅ are present, the two or more R₉₀₅ are mutually the same or different;
when two or more R₉₀₆ are present, the two or more R₉₀₆ are mutually the same or different; and
when two or more R₉₀₇ are present, the two or more R₉₀₇ are mutually the same or different.

In an exemplary embodiment, the substituent for the substituted or unsubstituted group is a group selected from the group consisting of an alkyl group having 1 to 50 carbon atoms, an aryl group having 6 to 50 ring carbon atoms, and a heterocyclic group having 5 to 50 ring atoms.

In an exemplary embodiment, the substituent for the substituted or unsubstituted group is a group selected from the group consisting of an alkyl group having 1 to 18 carbon atoms, an aryl group having 6 to 18 ring carbon atoms, and a heterocyclic group having 5 to 18 ring atoms.

Specific examples of the above optional substituent are the same as the specific examples of the substituent described in the above under the subtitle “Substituent Mentioned Herein.”

Unless otherwise specified herein, adjacent ones of the optional substituents may form a “saturated ring” or an “unsaturated ring,” preferably a substituted or unsubstituted saturated five-membered ring, a substituted or unsubstituted saturated six-membered ring, a substituted or unsubstituted unsaturated five-membered ring, or a substituted or unsubstituted unsaturated six-membered ring, more preferably a benzene ring.

Unless otherwise specified herein, the optional substituent may further include a substituent.

Examples of the substituent for the optional substituent are the same as the examples of the optional substituent.

Herein, numerical ranges represented by “AA to BB” represent a range whose lower limit is the value (AA) recited before “to” and whose upper limit is the value (BB) recited after “to.”

Herein, a numerical formula represented by “A≥B” means that the value A is equal to the value B, or the value A is larger than the value B.

Herein, a numerical formula represented by “A≤B” means that the value A is equal to the value B, or the value A is smaller than the value B.

### First Exemplary Embodiment

### Compound

The compound according to a first exemplary embodiment is a compound represented by a formula (1) below.

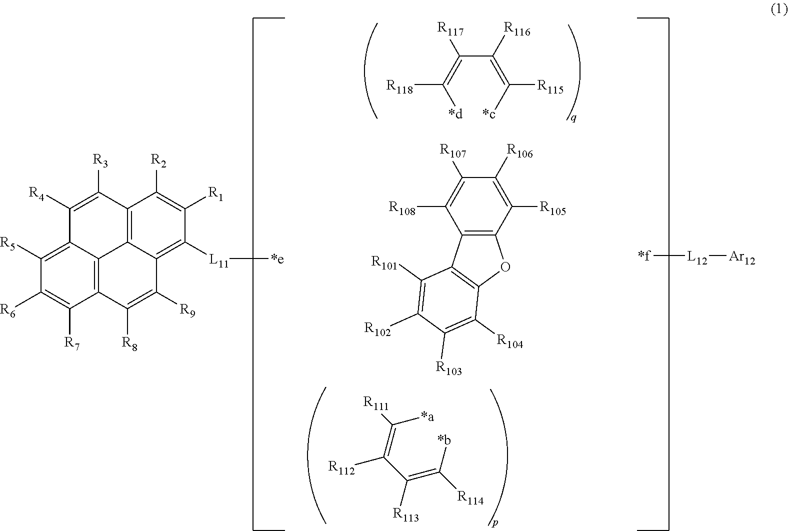

In the formula (1):
R₁ to R₉, R₁₀₁ to R₁₀₈, and R₁₁₁ to R₁₁₈ are each independently a hydrogen atom, a substituted or unsubstituted aryl group having 6 to 20 ring carbon atoms, or a substituted or unsubstituted heterocyclic group having 5 to 21 ring atoms;
Ar₁₂ is a substituted or unsubstituted aryl group having 10 to 30 ring carbon atoms, or a substituted or unsubstituted heterocyclic group having 9 to 31 ring atoms;
the substituted aryl group represented by Ar₁₂ has or does not have, as a substituent, at least one group selected from the group consisting of a substituted or unsubstituted aryl group having 6 to 20 ring carbon atoms and a substituted or unsubstituted heterocyclic group having 5 to 21 ring atoms;
the substituted heterocyclic group represented by Ar₁₂ has or does not have, as a substituent, at least one selected from the group consisting of a substituted or unsubstituted aryl group having 6 to 20 ring carbon atoms and a substituted or unsubstituted heterocyclic group having 5 to 21 ring atoms;
L₁₁ and L₁₂ are each independently a single bond, a substituted or unsubstituted arylene group having 6 to 10 ring carbon atoms, or a substituted or unsubstituted divalent heterocyclic group having 5 to 13 ring atoms;
p is 0 or 1;
q is 0 or 1; and
p+q is 1 or 2,
when p is 1, one of R₁₀₁ and R₁₀₂, R₁₀₂ and R₁₀₃, or R₁₀₃ and R₁₀₄ is a single bond bonded to *a, and the other of R₁₀₁ and R₁₀₂, R₁₀₂ and R₁₀₃, or R₁₀₃ and R₁₀₄ is a single bond bonded to *b;
when q is 0, of two selected from R₁₀₅ to R₁₀₈, one is a single bond bonded to *e, and the other is a single bond bonded to *f; and
when q is 1, one of R₁₀₅ and R₁₀₆, R₁₀₆ and R₁₀₇, or R₁₀₇ and R₁₀₈ is a single bond bonded to *c, and the other of R₁₀₅ and R₁₀₆, R₁₀₆ and R₁₀₇, or R₁₀₇ and R₁₀₈ is a single bond bonded to *d, and of two selected from R₁₀₅ to R₁₀₈ not being the single bonds bonded to *c and *d and R₁₁₅ to R₁₁₈, one is a single bond bonded to *e, and the other is a single bond bonded to *f.

The compound according to the first exemplary embodiment (the compound represented by the formula (1)) has, in a molecule, structures represented by formulae (1a), (1b), and (1c) below.

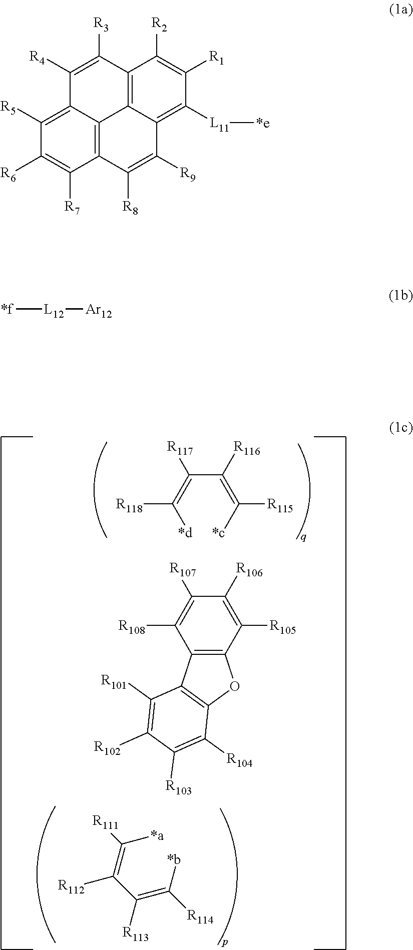

In the formulae (1a), (1b), and (1c), R₁ to R₉, R₁₀₁ to R₁₀₈, R₁₁₁ to R₁₁₈, Ar₁₂, L₁₁, L₁₂, p, q, *a, *b, *c, *d, *e, and *f are as defined in the formula (1).

In the compound according to the first exemplary embodiment, the structure represented by the formula (1c) is bonded to the structure represented by the formula (1a) (pyrene structure), and furthermore the structure represented by the formula (1b) (fused aryl structure having 10 or more ring atoms or fused heterocyclic structure having 10 or more ring atoms) is bonded to the structure represented by the formula (1c).

Having such a molecular structure, the compound according to the first exemplary embodiment has a singlet energy within a particular range (e.g., a singlet energy in a range from 2.95 eV to 3.25 eV) and has improved durability in an excited state.

Accordingly, when the compound according to the first exemplary embodiment is used for an organic layer of an organic EL device, the organic EL device has a prolonged lifetime.

In the compound according to the first exemplary embodiment, the structure represented by the formula (1a) (pyrene structure) and the structure represented by the formula (1b) (fused aryl structure having 10 or more ring atoms or fused heterocyclic structure having 10 or more ring atoms) are bonded to the same single monocyclic ring in the structure represented by the formula (1c), or the structure represented by the formula (1b) is bonded to a monocyclic ring (six-membered ring) adjacent to a monocyclic ring bonded to the structure represented by the formula (1a) (pyrene structure).

Consequently, the compound according to the first exemplary embodiment has a round molecular shape and thus is less likely to undergo intermolecular interaction.

Accordingly, when the compound according to the first exemplary embodiment is used for an organic layer of an organic EL device, chromaticity is expected to improve.

In the compound according to the first exemplary embodiment, when p is 1, “one of R₁₀₁ and R₁₀₂, R₁₀₂ and R₁₀₃, or R₁₀₃ and R₁₀₄ is a single bond bonded to *a, and the other of R₁₀₁ and R₁₀₂, R₁₀₂ and R₁₀₃, or R₁₀₃ and R₁₀₄ is a single bond bonded to *b” means that:
one of R₁₀₁ and R₁₀₂ is a single bond bonded to *a, and the other of R₁₀₁ and R₁₀₂ is a single bond bonded to *b;
one of R₁₀₂ and R₁₀₃ is a single bond bonded to *a, and the other of R₁₀₂ and R₁₀₃ is a single bond bonded to *b; or
one of R₁₀₃ and R₁₀₄ is a single bond bonded to *a, and the other of R₁₀₃ and R₁₀₄ is a single bond bonded to *b.

In the compound according to the first exemplary embodiment, when q is 1, “one of R₁₀₅ and R₁₀₆, R₁₀₆ and R₁₀₇, or R₁₀₇ and R₁₀₈ is a single bond bonded to *c, and the other of R₁₀₅ and R₁₀₆, R₁₀₆ and R₁₀₇, or R₁₀₇ and R₁₀₈ is a single bond bonded to *d” means that:
one of R₁₀₅ and R₁₀₆ is a single bond bonded to *c, and the other of R₁₀₅ and R₁₀₆ is a single bond bonded to *d;
one of R₁₀₆ and R₁₀₇ is a single bond bonded to *c, and the other of R₁₀₆ and R₁₀₇ is a single bond bonded to *d; or
one of R₁₀₇ and R₁₀₈ is a single bond bonded to *c, and the other of R₁₀₇ and R₁₀₈ is a single bond bonded to *d.

In the first exemplary embodiment, the compound represented by the formula (1) is preferably represented by a formula (10) below.

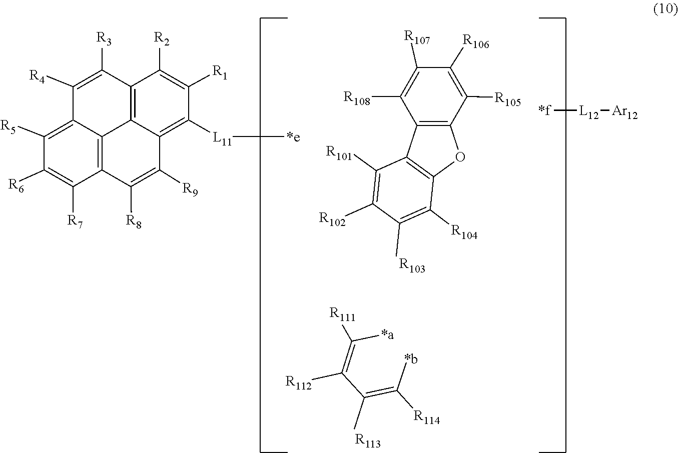

In the formula (10), R₁ to R₉, R₁₀₁ to R₁₀₈, R₁₁₁ to R₁₁₄, Ar₁₂, L₁₁, L₁₂, *a, *b, *e, and *f are as defined in the formula (1).

In the first exemplary embodiment, the compound represented by the formula (1) is also preferably represented by a formula (11) below.

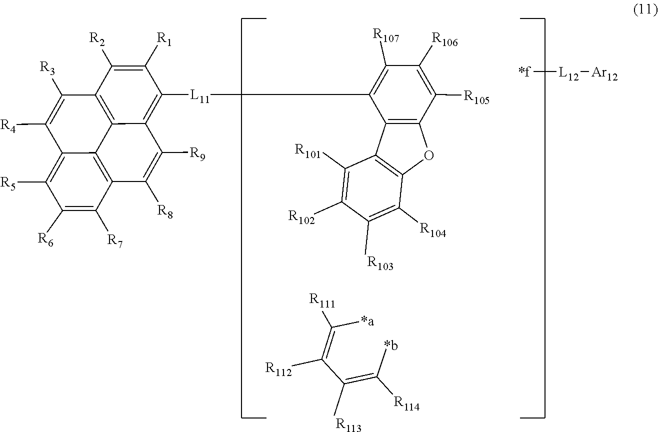

In the formula (11), R₁ to R₉, R₁₀₁ to R₁₀₇, R₁₁₁ to R₁₁₄, Ar₁₂, L₁₁, L₁₂, *a, *b, and *f are as defined in the formula (1).

In the first exemplary embodiment, R₁₀₅ or R₁₀₆ in the compound represented by the formula (1) is preferably a single bond bonded to *f.

In the first exemplary embodiment, preferably, one of R₁₀₂ and R₁₀₃ in the compound represented by the formula (1) is a single bond bonded to *a, and the other of R₁₀₂ and R₁₀₃ is a single bond bonded to *b.

When one of R₁₀₂ and R₁₀₃ in the formula (11) is a single bond bonded to *a and the other of R₁₀₂ and R₁₀₃ is a single bond bonded to *b, the compound according to the first exemplary embodiment is represented by a formula (111) below.

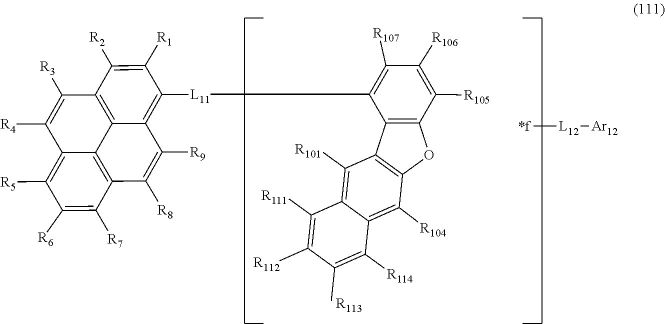

In the formula (111), R₁ to R₉, R₁₀₁, R₁₀₄, R₁₀₅ to R₁₀₇, R₁₁₁ to R₁₁₄, Ar₁₂, L₁₁, L₁₂, and *f are as defined in the formula (1).

In the first exemplary embodiment, preferably, one of R₁₀₃ and R₁₀₄ in the compound represented by the formula (1) is a single bond bonded to *a, and the other of R₁₀₃ and R₁₀₄ is a single bond bonded to *b.

When one of R₁₀₃ and R₁₀₄ in the formula (11) is a single bond bonded to *a and the other of R₁₀₃ and R₁₀₄ is a single bond bonded to *b, the compound according to the first exemplary embodiment is represented by a formula (112) below.

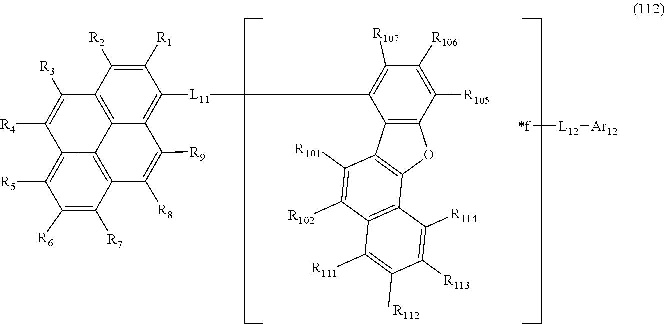

In the formula (112), R₁ to R₉, R₁₀₁, R₁₀₂, R₁₀₅ to R₁₀₇, R₁₁₁ to R₁₁₄, Ar₁₂, L₁₁, L₁₂, and *f are as defined in the formula (1).

In the first exemplary embodiment, Ar₁₂ in the compound represented by the formula (1) is preferably an aryl group derived from fusion of four or less monocyclic rings.

In the compound according to the first exemplary embodiment, Ar₁₂ in the compound represented by the formula (1) is preferably an aryl group derived from fusion of two, three, or four monocyclic rings.

In a first host material, for instance, the pyrenyl group and benzanthryl group are aryl groups derived from fusion of four monocyclic rings (six-membered rings), the anthryl group and phenanthryl group are aryl groups derived from fusion of three monocyclic rings (six-membered rings), and the dibenzofuranyl group and dibenzothienyl group are heterocyclic groups derived from fusion of three monocyclic rings (two six-membered rings and one five-membered ring).

In the first exemplary embodiment, the aryl group represented by Ar₁₂ in the compound represented by the formula (1) also preferably has, as a substituent, an aryl group having 6 to 10 ring carbon atoms.

In the first exemplary embodiment, Ar₁₂ in the compound represented by the formula (1) is also preferably an unsubstituted aryl group having 10 to 30 ring carbon atoms.

In the compound according to the first exemplary embodiment, Ar₁₂ is also preferably a group represented by a formula (1100) or (1200) below.

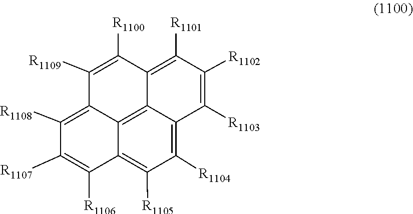

In the formula (1100), one of R₁₁₀₁ to R₁₁₁₀ represents a bonding position to L₁₂, and R₁₁₀₁ to R₁₁₁₀ not being the bonding position to L₁₂ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 17 ring carbon atoms, or a substituted or unsubstituted heterocyclic group having 5 to 17 ring atoms.

In the compound according to the first exemplary embodiment, R₉₀₁, R₉₀₂, R₉₀₃, R₉₀₄, R₉₀₅, R₉₀₆, R₉₀₇, R₈₀₁, and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

In the compound according to the first exemplary embodiment, Ar₁₂ is also preferably a group represented by a formula (1111), a formula (1112), or a formula (1113) below.

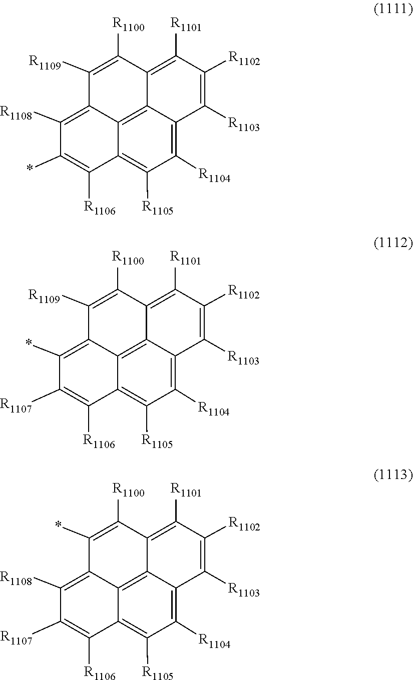

In the formulae (1111), (1112), and (1113), R₁₁₀₁ to R₁₁₁₀ respectively represent the same as R₁₁₀₁ to R₁₁₁₀ in the formula (1100), and * represents a bonding position to L₁₂.

In the compound according to the first exemplary embodiment, R₁₁₀₁ to R₁₁₁₀ not being the bonding position to L₁₂ are preferably each independently a hydrogen atom or a substituted or unsubstituted aryl group having 6 to 14 ring carbon atoms, preferably a hydrogen atom or a substituted or unsubstituted aryl group having 6 to 10 ring carbon atoms, and further preferably a hydrogen atom or a substituted or unsubstituted phenyl group.

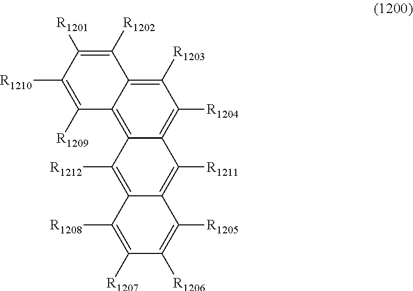

In the formula (1200), one of R₁₂₀₁ to R₁₂₁₂ represents a bonding position to L₁₂, and R₁₂₀₁ to R₁₂₁₂ not being the bonding position to L₁₂ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 17 ring carbon atoms, or a substituted or unsubstituted heterocyclic group having 5 to 17 ring atoms.

In the compound according to the first exemplary embodiment, R₁₂₁₁ or R₁₂₁₂ in the formula (1200) is preferably a bonding position to L₁₂.

In the compound according to the first exemplary embodiment, R₁₂₁₁ in the formula (1200) is preferably a bonding position to L₁₂.

In the compound according to the first exemplary embodiment, Ar₁₂ is also preferably a group represented by a formula (1211) or a formula (1212) below.

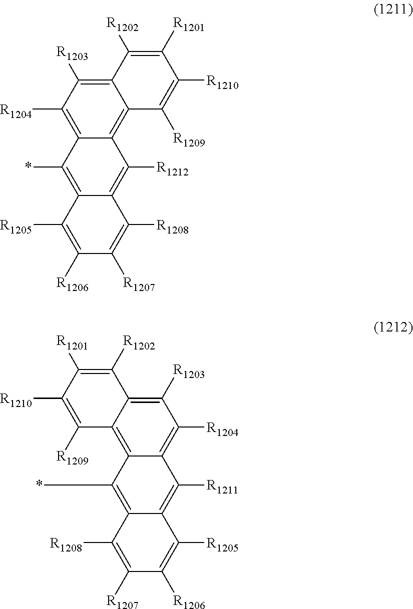

In the formula (1211) and the formula (1212), R₁₂₀₁ to R₁₂₁₂ respectively represent the same as R₁₂₀₁ to R₁₂₁₂ in the formula (1200), and * represents a bonding position to L₁₂.

In the compound according to the first exemplary embodiment, R₁₂₀₁ to R₁₂₁₂ not being the bonding position to L₁₂ are preferably each independently a hydrogen atom or a substituted or unsubstituted aryl group having 6 to 14 ring carbon atoms, preferably a hydrogen atom or a substituted or unsubstituted aryl group having 6 to 10 ring carbon atoms, and further preferably a hydrogen atom or a substituted or unsubstituted phenyl group.

In the compound according to the first exemplary embodiment, R₁₁₀₁ to R₁₁₁₀ and R₁₂₀₁ to R₁₂₁₂ not being the bonding position to L₁₂ are each also preferably a hydrogen atom.

In the first exemplary embodiment, L₁₁ in the compound represented by the formula (1) is also preferably a single bond or a substituted or unsubstituted arylene group having 6 to 10 ring carbon atoms.

In the first exemplary embodiment, L₁₁ in the compound represented by the formula (1) is also preferably a single bond.

In the first exemplary embodiment, L₁₂ in the compound represented by the formula (1) is preferably a single bond or a substituted or unsubstituted arylene group having 6 to 10 ring carbon atoms.

In the first exemplary embodiment, L₁₂ in the compound represented by the formula (1) is preferably a single bond, a substituted or unsubstituted phenylene group, or a substituted or unsubstituted naphthylene group.

In the first exemplary embodiment, R₁ to R₉, R₁₀₁ to R₁₀₈ not being the single bonds, and R₁₁₁ to R₁₁₈ not being the single bonds in the compound represented by the formula (1) are preferably each independently a hydrogen atom or a substituted or unsubstituted aryl group having 6 to 10 ring carbon atoms.

In the first exemplary embodiment, R₁ to R₉, R₁₀₁ to R₁₀₈ not being the single bonds, and R₁₁₁ to R₁₁₈ not being the single bonds in the compound represented by the formula (1) are each preferably a hydrogen atom.

In the compound according to the first exemplary embodiment, the groups specified to be “substituted or unsubstituted” are each preferably an “unsubstituted” group.

Method for Producing Compound According to First Exemplary Embodiment

The compound according to the first exemplary embodiment can be produced according to a synthesis method described in Examples given later or based on the synthesis method through a known alternative reaction using a known material(s) tailored for the target compound.

Specific Examples of Compound According to First Exemplary Embodiment Specific examples of the compound according to the first exemplary embodiment include the following compounds.

However, the invention is by no means limited to the specific examples.

Herein, a deuterium atom is denoted as D in formulae, and a protium atom is denoted as H or omitted.

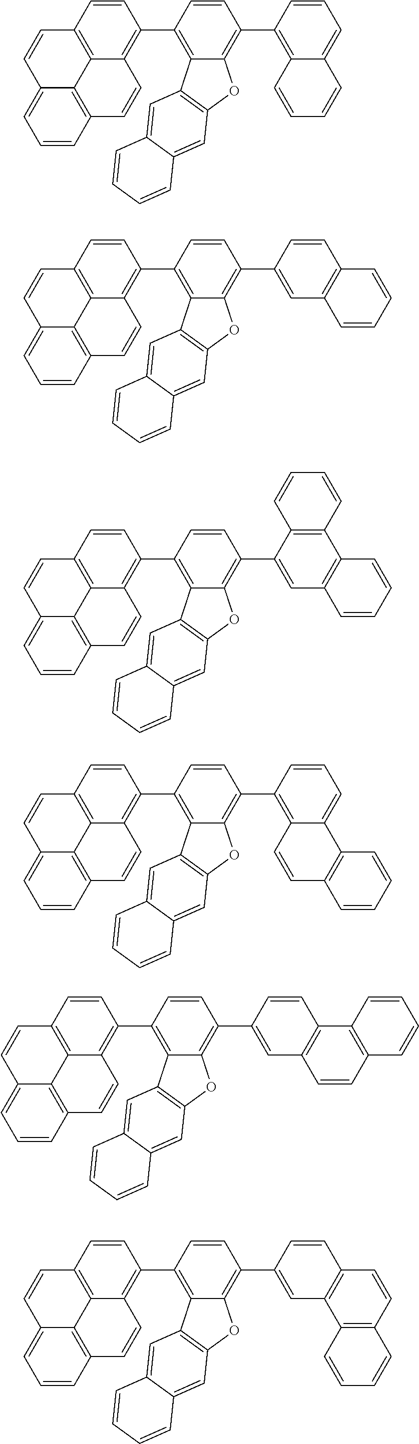

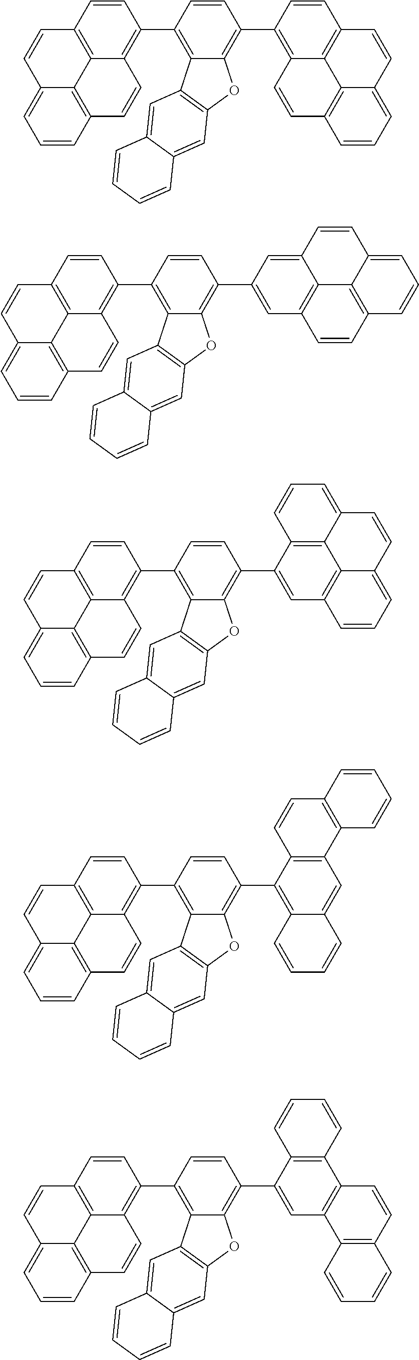 [SMILES: c1ccc2cc3c(cc2c1)oc1c(-c2ccc4ccc5cccc6ccc2c4c56)ccc(-c2ccc4ccc5cccc6ccc2c4c56)c13 ; c1ccc2cc3c(cc2c1)oc1c(-c2cc4ccc5cccc6ccc(c2)c4c56)ccc(-c2ccc4ccc5cccc6ccc2c4c56)c13 ; c1ccc2cc3c(cc2c1)oc1c(-c2cc4cccc5ccc6cccc2c6c54)ccc(-c2ccc4ccc5cccc6ccc2c4c56)c13 ; c1ccc2cc3c(cc2c1)oc1c(-c2c4ccccc4cc4c2ccc2ccccc24)ccc(-c2ccc4ccc5cccc6ccc2c4c56)c13 ; c1ccc2cc3c(cc2c1)oc1c(-c2cc4c5ccccc5ccc4c4ccccc24)ccc(-c2ccc4ccc5cccc6ccc2c4c56)c13]

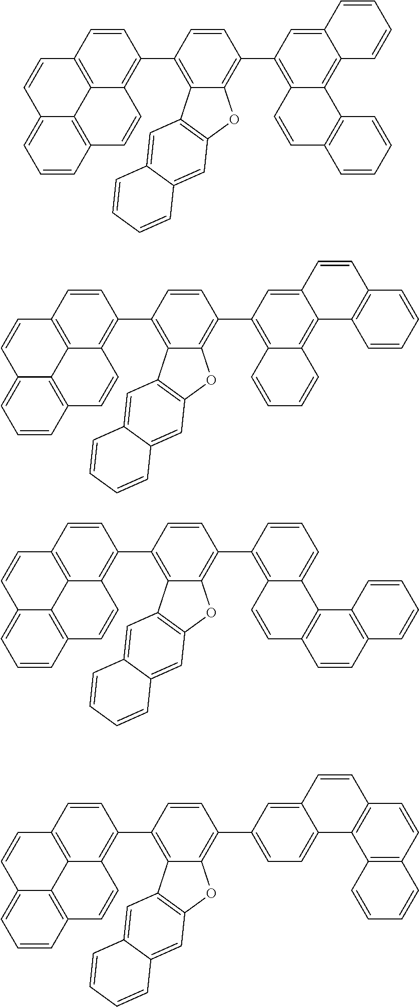

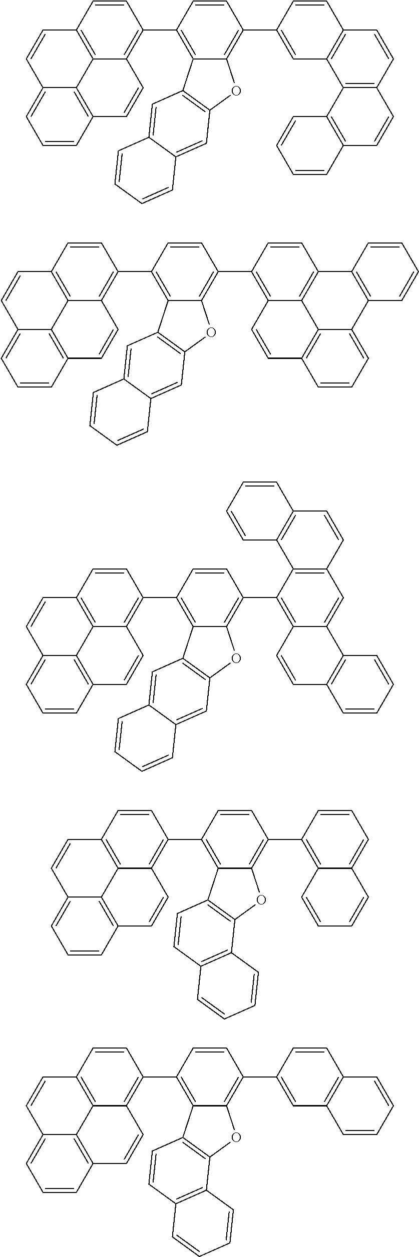

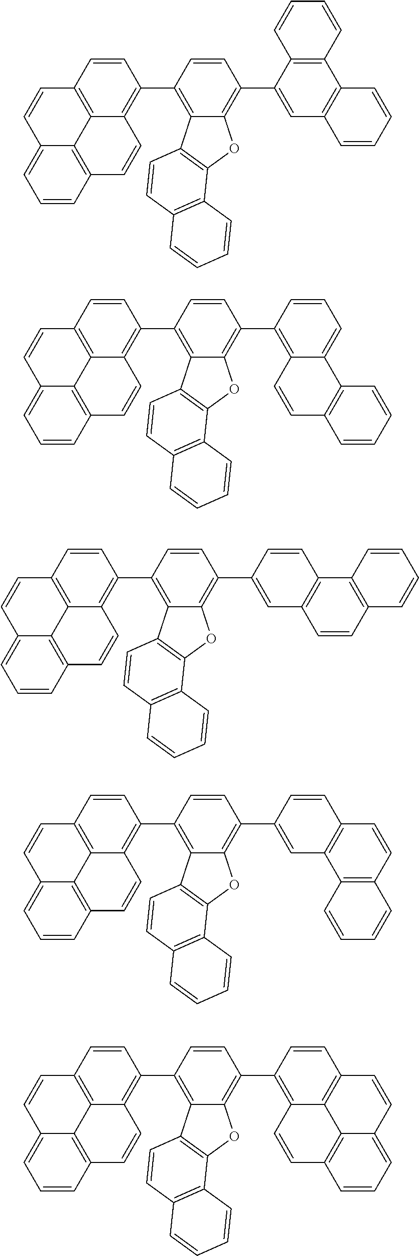

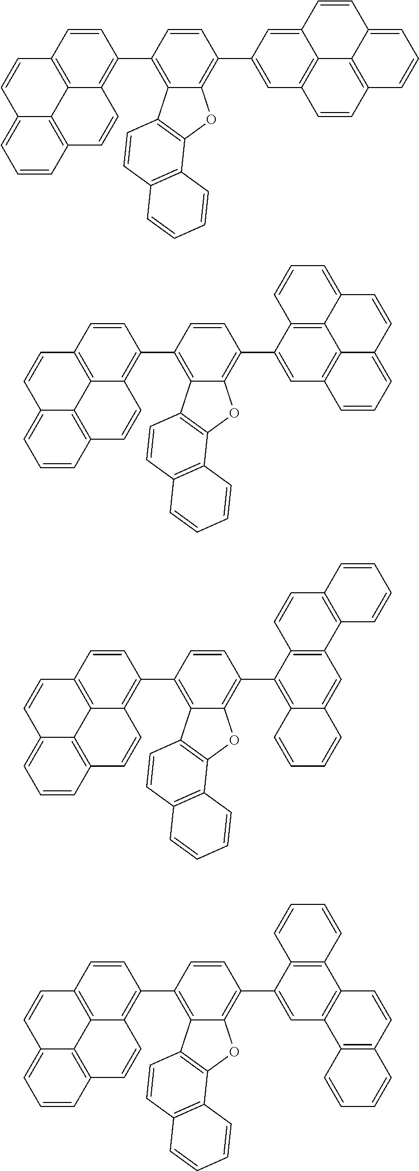 [SMILES: c1ccc2c(c1)ccc1c2oc2c(-c3cc4ccc5cccc6ccc(c3)c4c56)ccc(-c3ccc4ccc5cccc6ccc3c4c56)c21 ; c1ccc2c(c1)ccc1c2oc2c(-c3cc4cccc5ccc6cccc3c6c54)ccc(-c3ccc4ccc5cccc6ccc3c4c56)c21 ; c1ccc2c(-c3ccc(-c4ccc5ccc6cccc7ccc4c5c67)c4c3oc3c5ccccc5ccc34)c3ccc4ccccc4c3cc2c1 ; c1ccc2c(c1)ccc1c3ccccc3c(-c3ccc(-c4ccc5ccc6cccc7ccc4c5c67)c4c3oc3c5ccccc5ccc34)cc21]

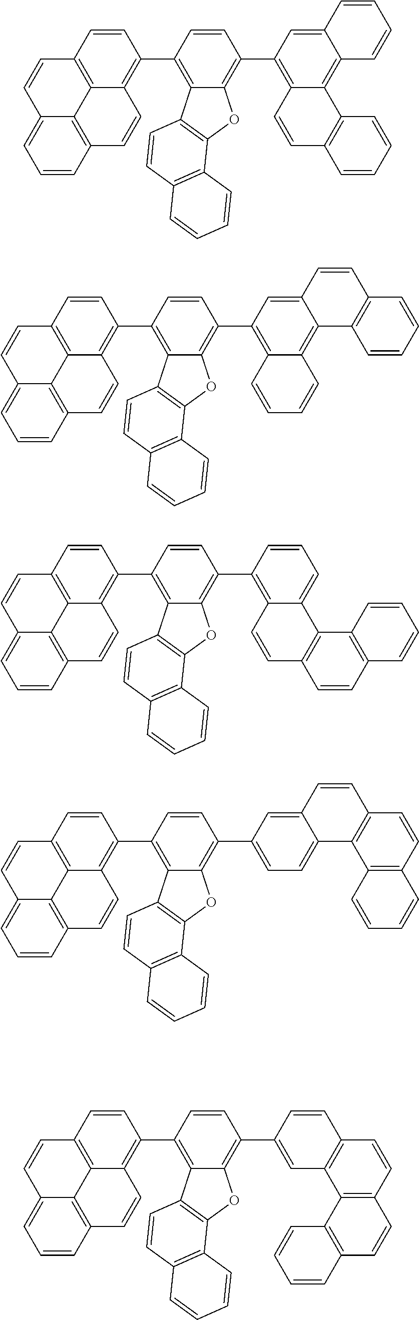

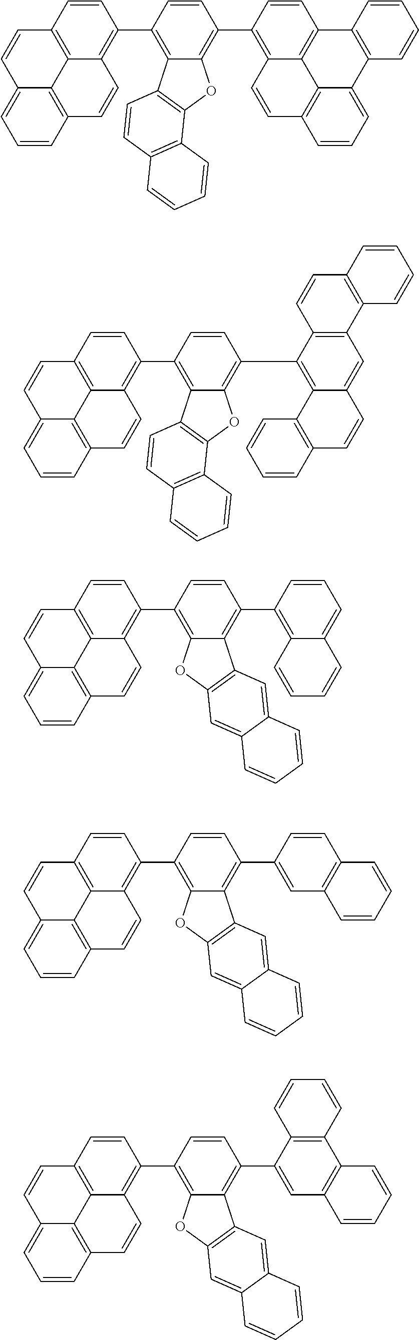

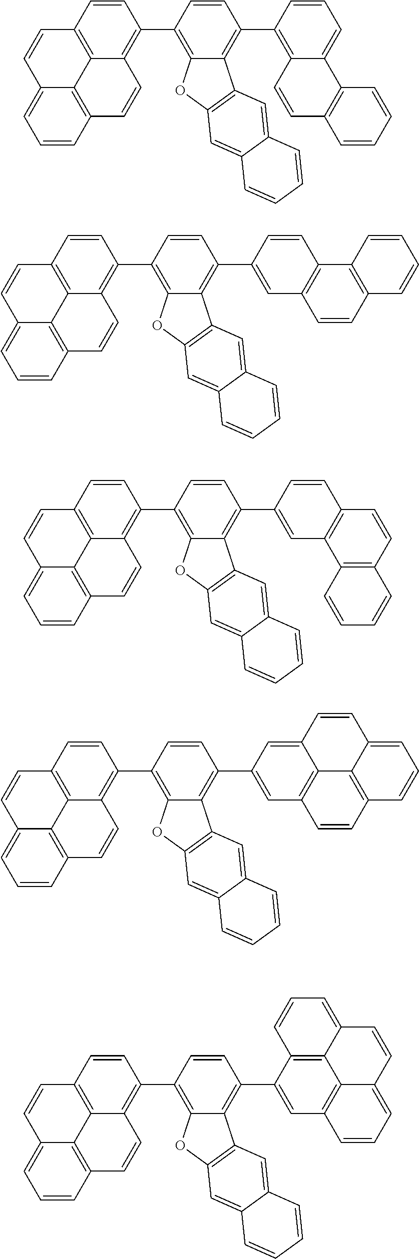

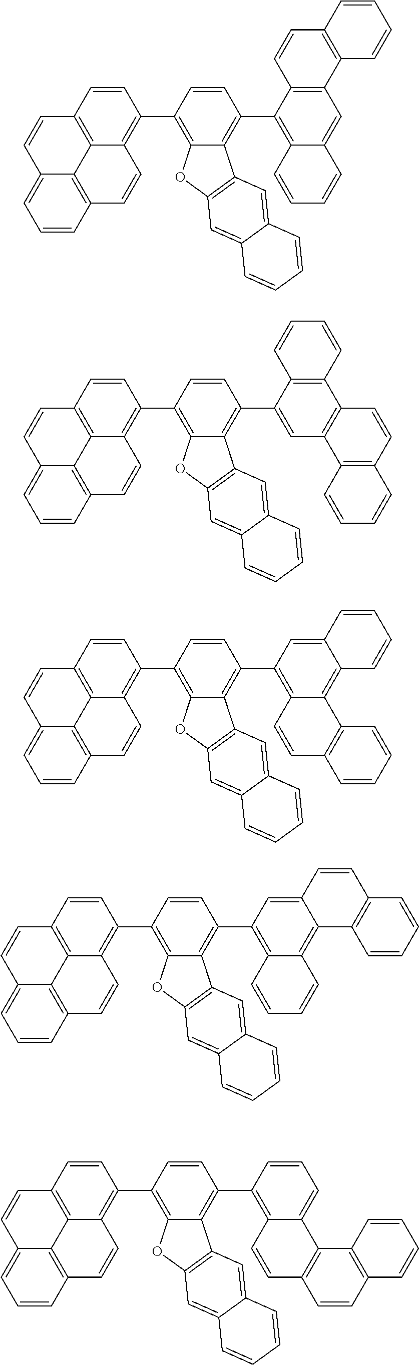

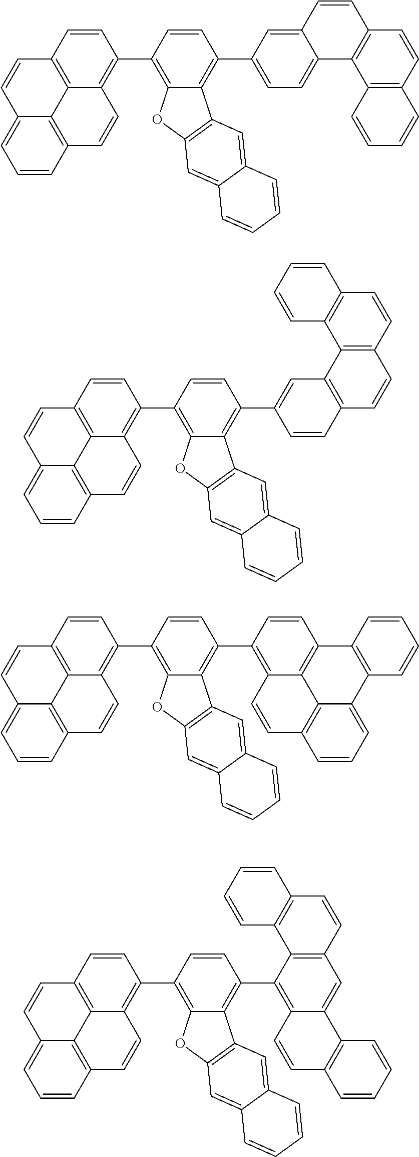

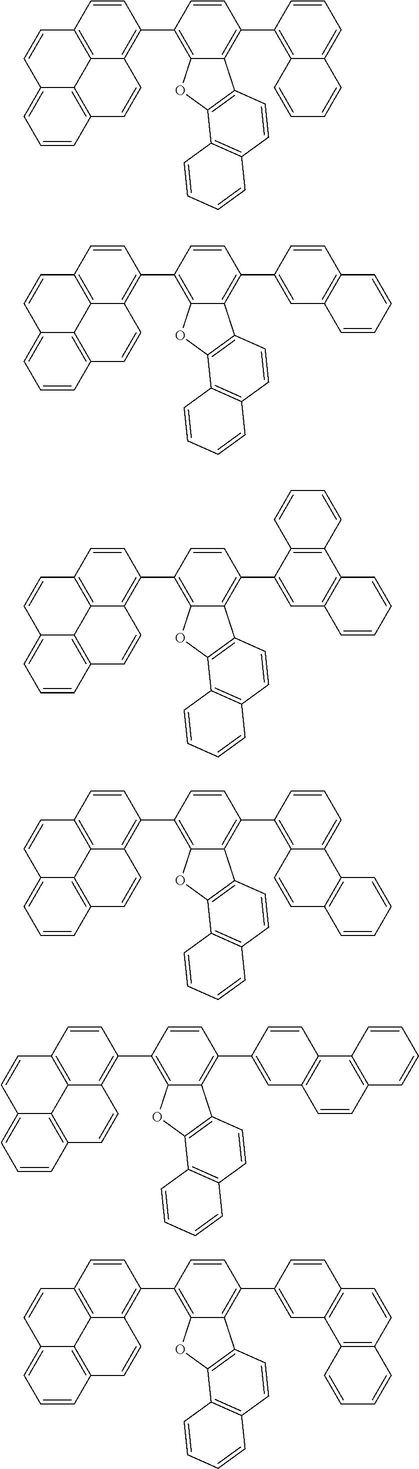

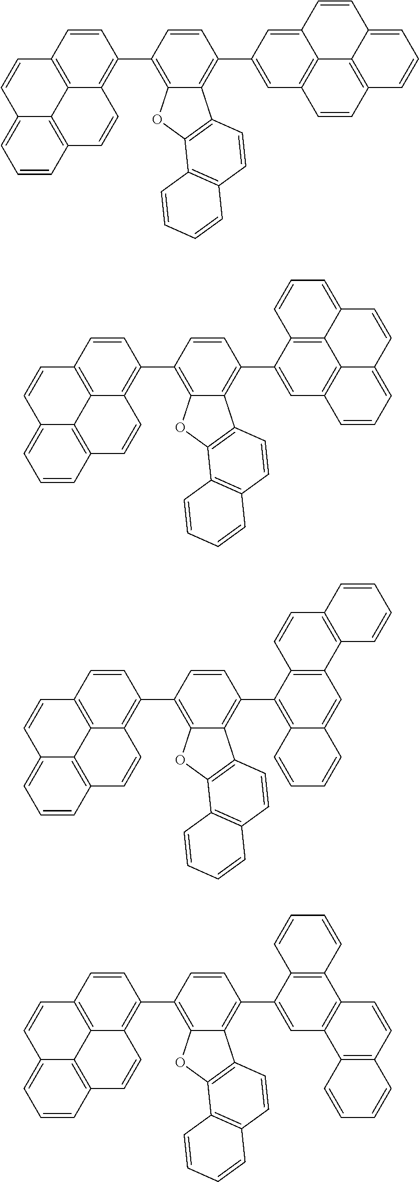 [SMILES: c1ccc2c(c1)ccc1c2oc2c(-c3ccc4ccc5cccc6ccc3c4c56)ccc(-c3cc4ccc5cccc6ccc(c3)c4c56)c21 ; c1ccc2c(c1)ccc1c2oc2c(-c3ccc4ccc5cccc6ccc3c4c56)ccc(-c3cc4cccc5ccc6cccc3c6c54)c21 ; c1ccc2c(-c3ccc(-c4ccc5ccc6cccc7ccc4c5c67)c4oc5c6ccccc6ccc5c34)c3ccc4ccccc4c3cc2c1 ; c1ccc2c(c1)ccc1c3ccccc3c(-c3ccc(-c4ccc5ccc6cccc7ccc4c5c67)c4oc5c6ccccc6ccc5c34)cc21]

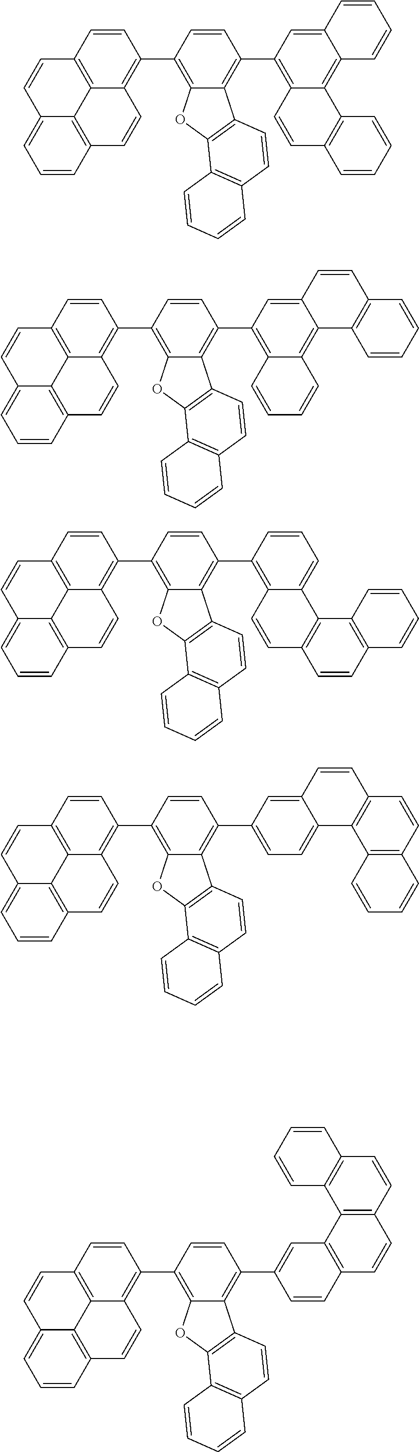

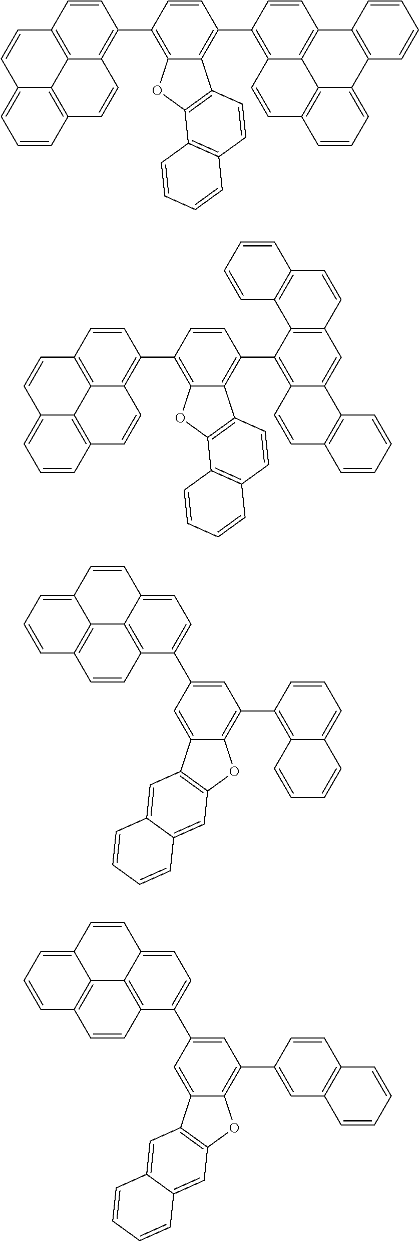

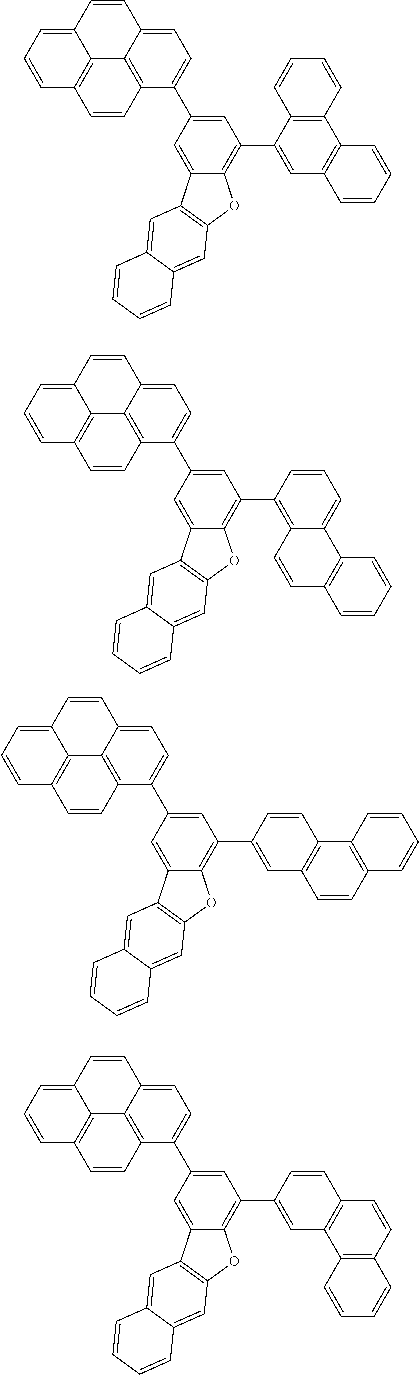

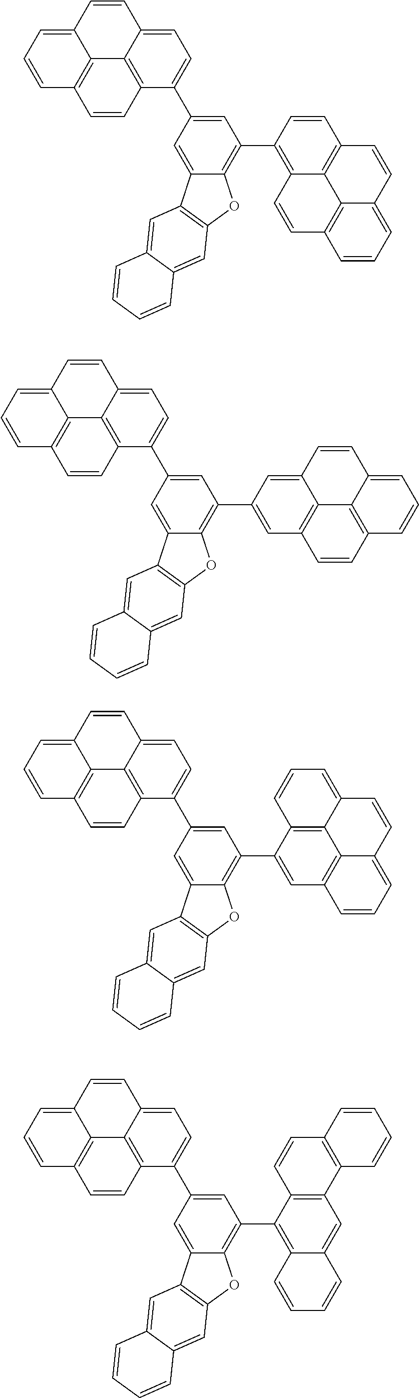

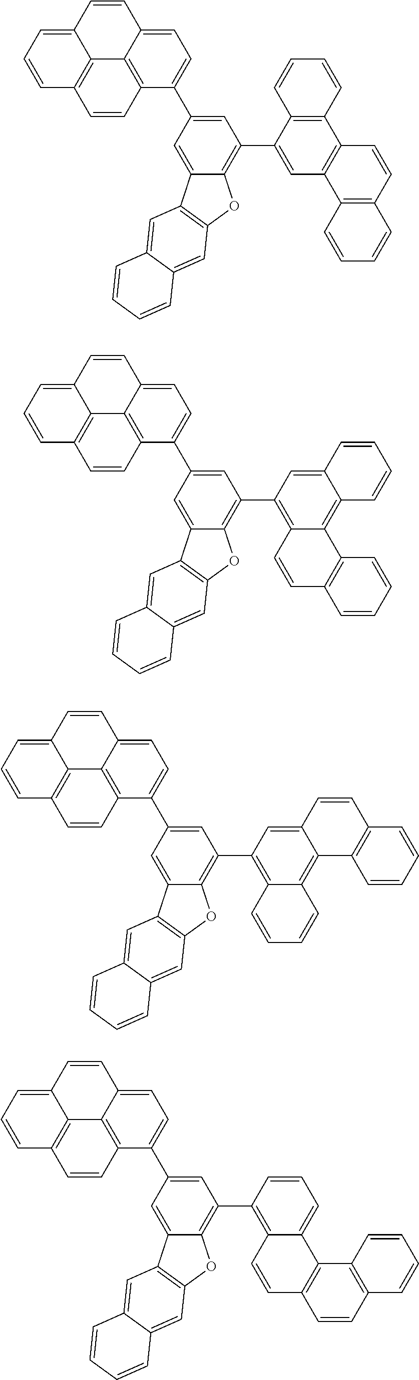

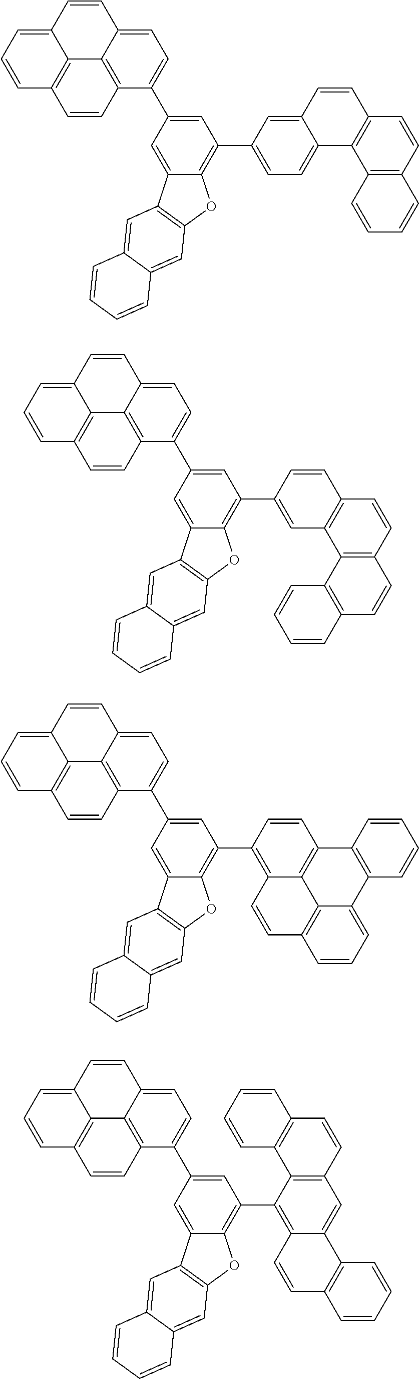

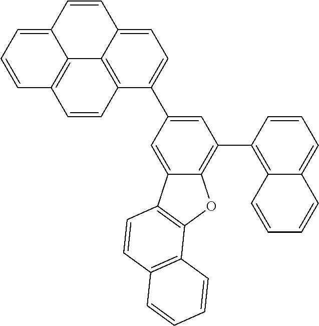 [SMILES: c1ccc2c(-c3cc(-c4ccc5ccc6cccc7ccc4c5c67)cc4c3oc3c5ccccc5ccc43)cccc2c1]

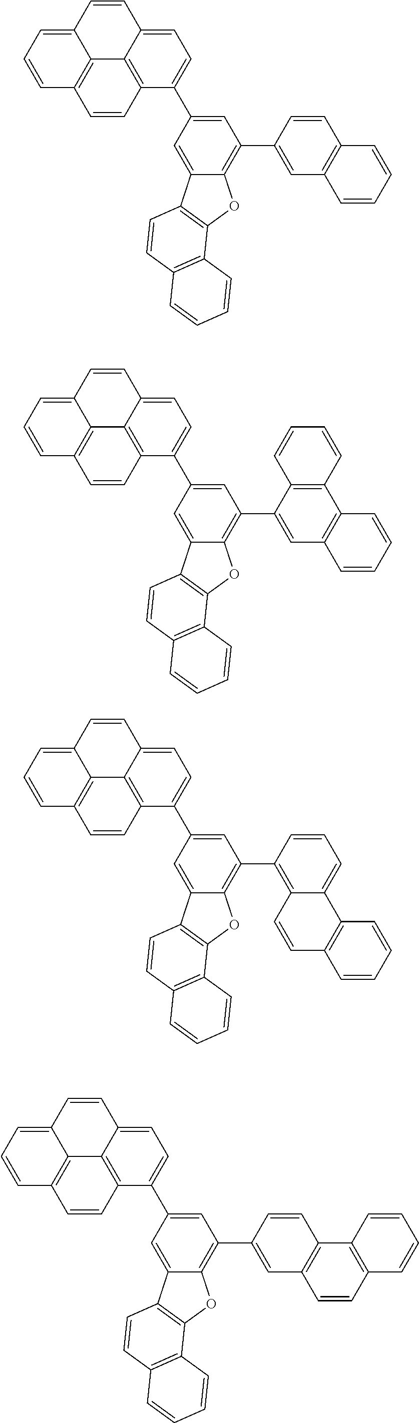 [SMILES: c1ccc2cc(-c3cc(-c4ccc5ccc6cccc7ccc4c5c67)cc4c3oc3c5ccccc5ccc43)ccc2c1 ; c1ccc2c(c1)cc(-c1cc(-c3ccc4ccc5cccc6ccc3c4c56)cc3c1oc1c4ccccc4ccc31)c1ccccc12 ; c1ccc2c(c1)ccc1c(-c3cc(-c4ccc5ccc6cccc7ccc4c5c67)cc4c3oc3c5ccccc5ccc43)cccc12 ; c1ccc2c(c1)ccc1cc(-c3cc(-c4ccc5ccc6cccc7ccc4c5c67)cc4c3oc3c5ccccc5ccc43)ccc12]

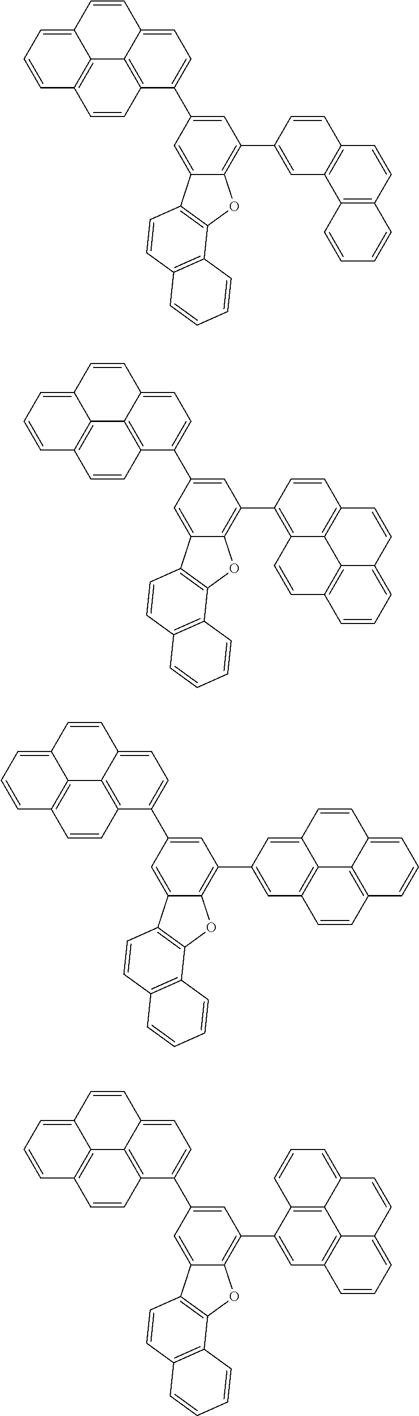 [SMILES: c1ccc2c(c1)ccc1ccc(-c3cc(-c4ccc5ccc6cccc7ccc4c5c67)cc4c3oc3c5ccccc5ccc43)cc12 ; c1ccc2c(c1)ccc1c3cc(-c4ccc5ccc6cccc7ccc4c5c67)cc(-c4ccc5ccc6cccc7ccc4c5c67)c3oc21 ; c1ccc2c(c1)ccc1c3cc(-c4ccc5ccc6cccc7ccc4c5c67)cc(-c4cc5ccc6cccc7ccc(c4)c5c67)c3oc21 ; c1ccc2c(c1)ccc1c3cc(-c4ccc5ccc6cccc7ccc4c5c67)cc(-c4cc5cccc6ccc7cccc4c7c65)c3oc21]

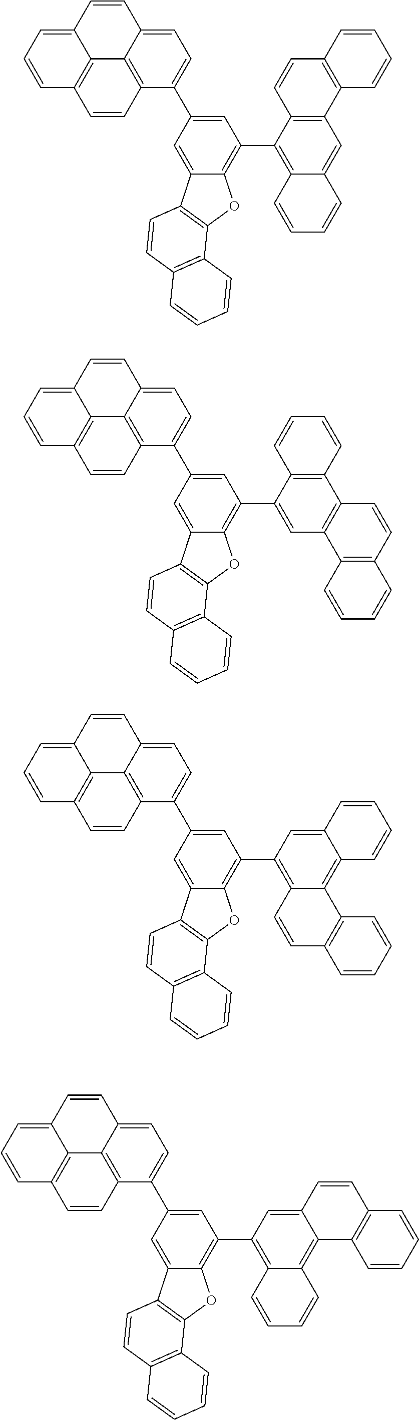 [SMILES: c1ccc2c(-c3cc(-c4ccc5ccc6cccc7ccc4c5c67)cc4c3oc3c5ccccc5ccc43)c3ccc4ccccc4c3cc2c1 ; c1ccc2c(c1)ccc1c3ccccc3c(-c3cc(-c4ccc5ccc6cccc7ccc4c5c67)cc4c3oc3c5ccccc5ccc43)cc21 ; c1ccc2c(c1)ccc1c3cc(-c4ccc5ccc6cccc7ccc4c5c67)cc(-c4cc5ccccc5c5c4ccc4ccccc45)c3oc21 ; c1ccc2c(c1)ccc1c3cc(-c4ccc5ccc6cccc7ccc4c5c67)cc(-c4cc5ccc6ccccc6c5c5ccccc45)c3oc21]

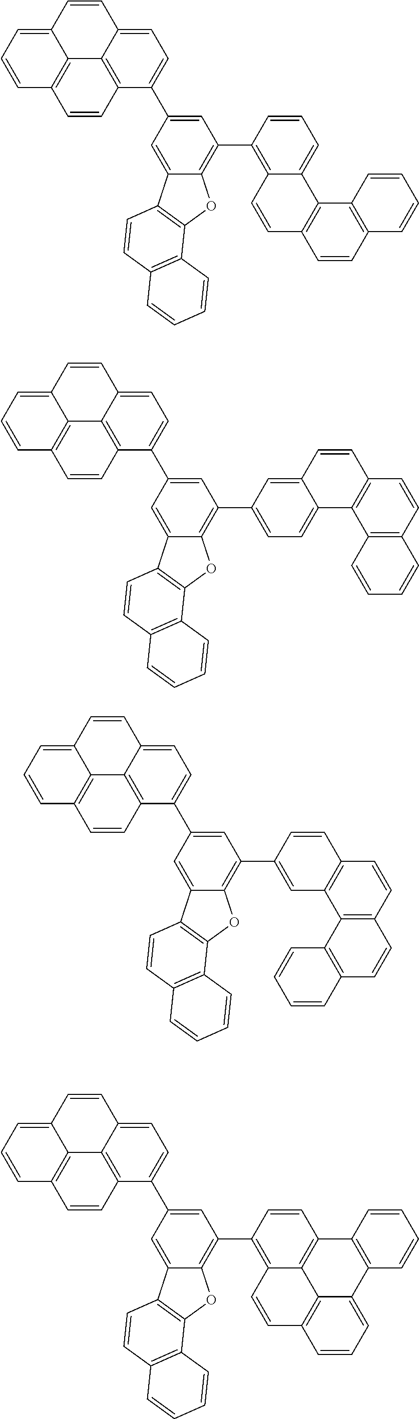 [SMILES: c1ccc2c(c1)ccc1c3cc(-c4ccc5ccc6cccc7ccc4c5c67)cc(-c4cccc5c4ccc4ccc6ccccc6c45)c3oc21 ; c1ccc2c(c1)ccc1c3cc(-c4ccc5ccc6cccc7ccc4c5c67)cc(-c4ccc5c(ccc6ccc7ccccc7c65)c4)c3oc21 ; c1ccc2c(c1)ccc1c3cc(-c4ccc5ccc6cccc7ccc4c5c67)cc(-c4ccc5ccc6ccc7ccccc7c6c5c4)c3oc21 ; c1ccc2c(c1)ccc1c3cc(-c4ccc5ccc6cccc7ccc4c5c67)cc(-c4ccc5c6ccccc6c6cccc7ccc4c5c76)c3oc21]

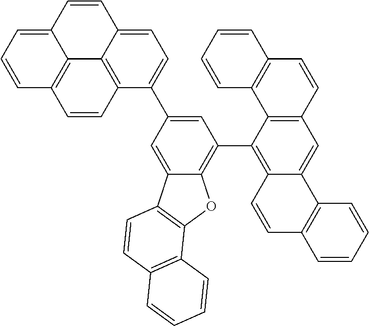 [SMILES: c1ccc2c(c1)ccc1c(-c3cc(-c4ccc5ccc6cccc7ccc4c5c67)cc4c3oc3c5ccccc5ccc43)c3c(ccc4ccccc43)cc12]

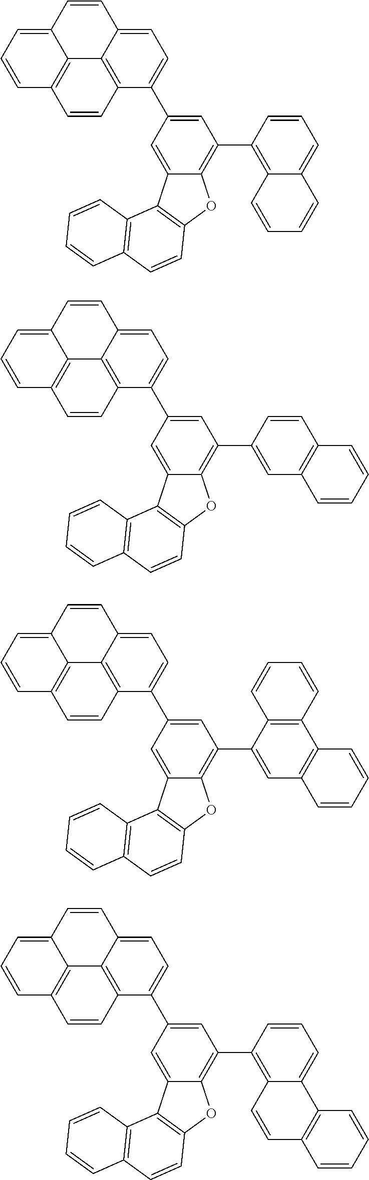

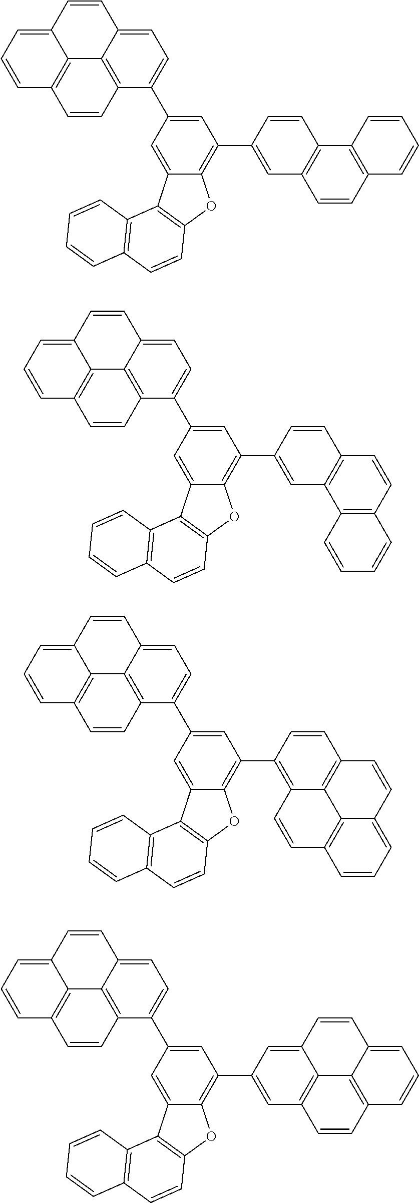

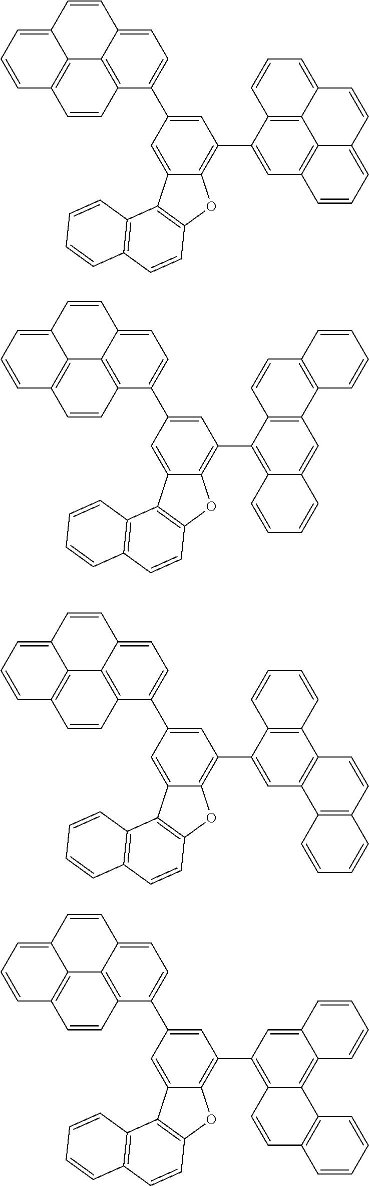

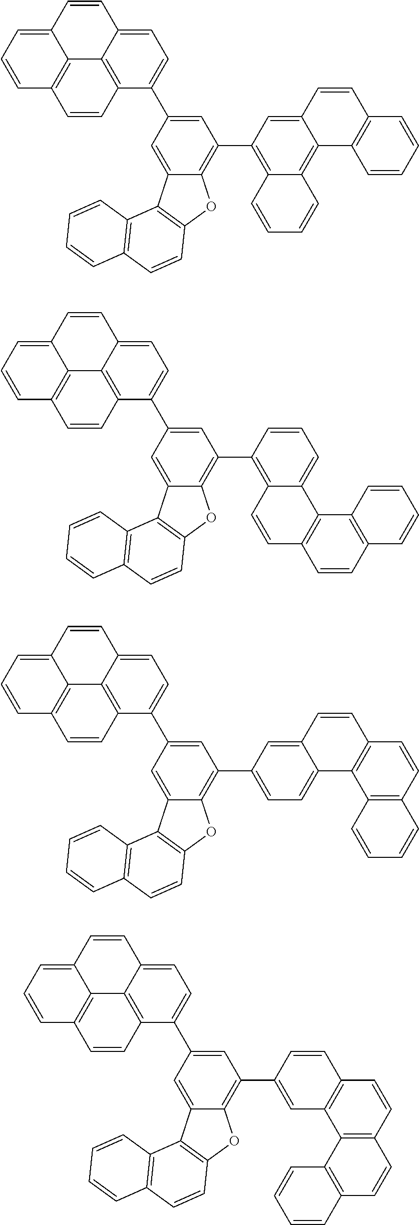

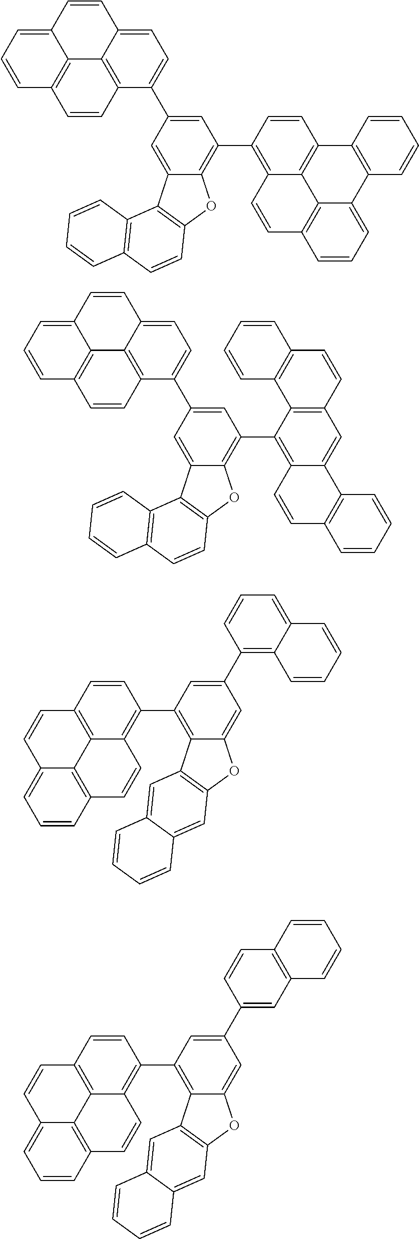

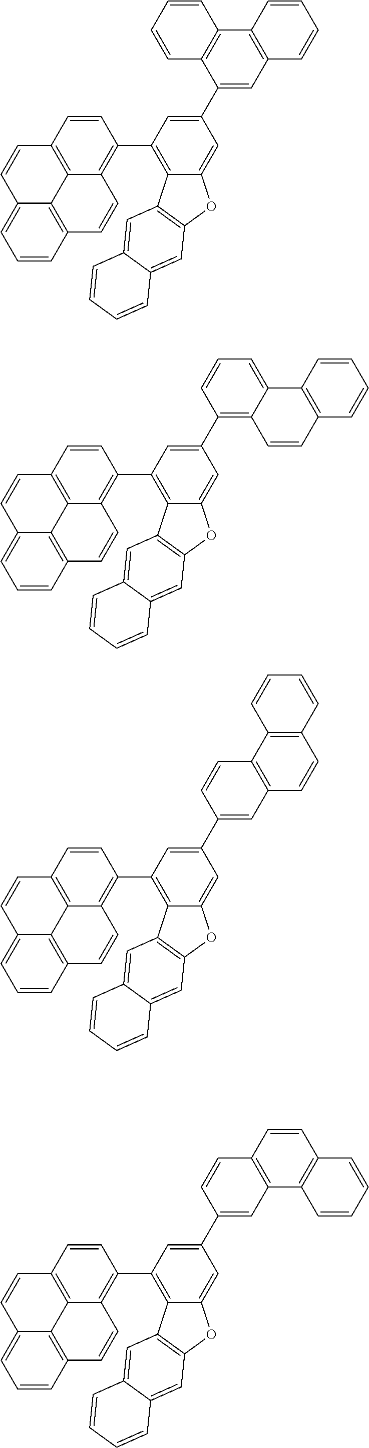 [SMILES: c1ccc2cc3c(cc2c1)oc1cc(-c2cc4ccccc4c4ccccc24)cc(-c2ccc4ccc5cccc6ccc2c4c56)c13 ; c1ccc2cc3c(cc2c1)oc1cc(-c2cccc4c2ccc2ccccc24)cc(-c2ccc4ccc5cccc6ccc2c4c56)c13 ; c1ccc2cc3c(cc2c1)oc1cc(-c2ccc4c(ccc5ccccc54)c2)cc(-c2ccc4ccc5cccc6ccc2c4c56)c13 ; c1ccc2cc3c(cc2c1)oc1cc(-c2ccc4ccc5ccccc5c4c2)cc(-c2ccc4ccc5cccc6ccc2c4c56)c13]

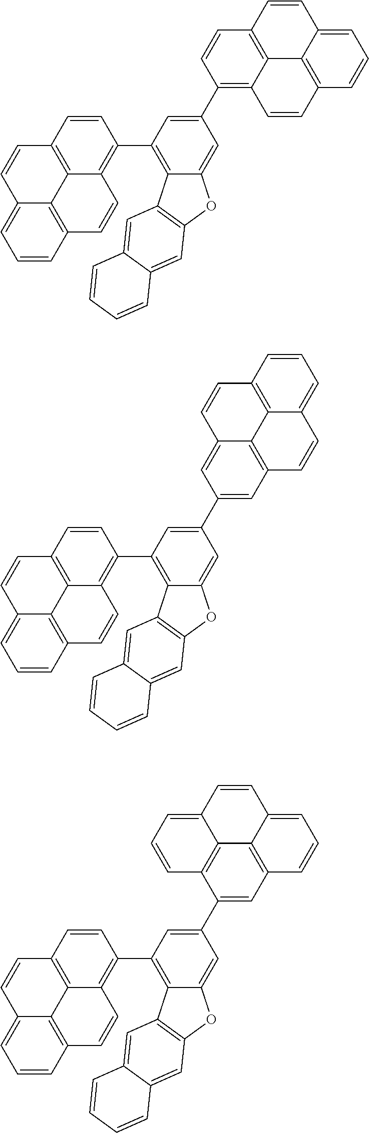 [SMILES: c1ccc2cc3c(cc2c1)oc1cc(-c2ccc4ccc5cccc6ccc2c4c56)cc(-c2ccc4ccc5cccc6ccc2c4c56)c13 ; c1ccc2cc3c(cc2c1)oc1cc(-c2cc4ccc5cccc6ccc(c2)c4c56)cc(-c2ccc4ccc5cccc6ccc2c4c56)c13 ; c1ccc2cc3c(cc2c1)oc1cc(-c2cc4cccc5ccc6cccc2c6c54)cc(-c2ccc4ccc5cccc6ccc2c4c56)c13]

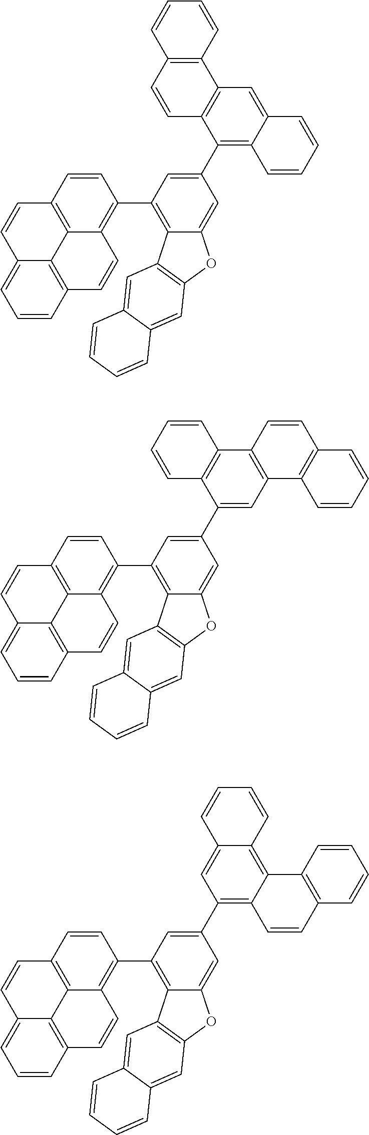 [SMILES: c1ccc2cc3c(cc2c1)oc1cc(-c2c4ccccc4cc4c2ccc2ccccc24)cc(-c2ccc4ccc5cccc6ccc2c4c56)c13 ; c1ccc2cc3c(cc2c1)oc1cc(-c2cc4c5ccccc5ccc4c4ccccc24)cc(-c2ccc4ccc5cccc6ccc2c4c56)c13 ; c1ccc2cc3c(cc2c1)oc1cc(-c2cc4ccccc4c4c2ccc2ccccc24)cc(-c2ccc4ccc5cccc6ccc2c4c56)c13]

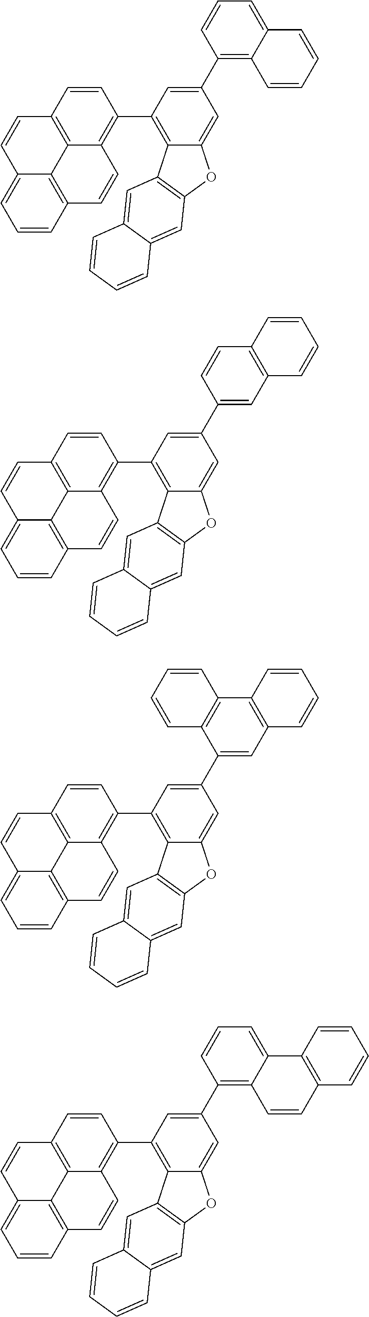 [SMILES: c1ccc2cc3c(cc2c1)oc1cc(-c2cccc4ccccc24)cc(-c2ccc4ccc5cccc6ccc2c4c56)c13 ; c1ccc2cc(-c3cc(-c4ccc5ccc6cccc7ccc4c5c67)c4c(c3)oc3cc5ccccc5cc34)ccc2c1 ; c1ccc2cc3c(cc2c1)oc1cc(-c2cc4ccccc4c4ccccc24)cc(-c2ccc4ccc5cccc6ccc2c4c56)c13 ; c1ccc2cc3c(cc2c1)oc1cc(-c2cccc4c2ccc2ccccc24)cc(-c2ccc4ccc5cccc6ccc2c4c56)c13]

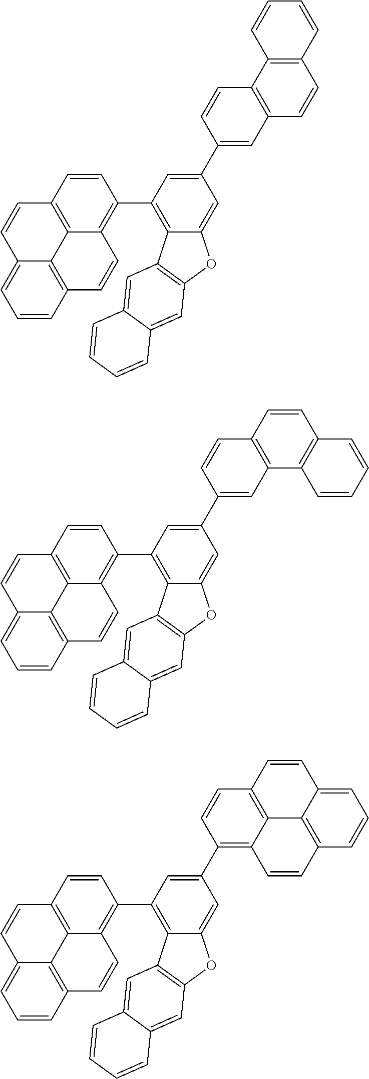 [SMILES: c1ccc2cc3c(cc2c1)oc1cc(-c2ccc4c(ccc5ccccc54)c2)cc(-c2ccc4ccc5cccc6ccc2c4c56)c13 ; c1ccc2cc3c(cc2c1)oc1cc(-c2ccc4ccc5ccccc5c4c2)cc(-c2ccc4ccc5cccc6ccc2c4c56)c13 ; c1ccc2cc3c(cc2c1)oc1cc(-c2ccc4ccc5cccc6ccc2c4c56)cc(-c2ccc4ccc5cccc6ccc2c4c56)c13]

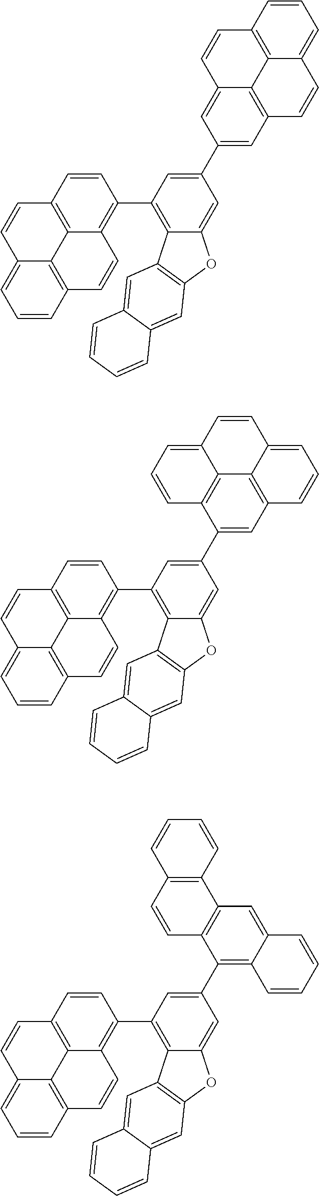 [SMILES: c1ccc2cc3c(cc2c1)oc1cc(-c2cc4ccc5cccc6ccc(c2)c4c56)cc(-c2ccc4ccc5cccc6ccc2c4c56)c13 ; c1ccc2cc3c(cc2c1)oc1cc(-c2cc4cccc5ccc6cccc2c6c54)cc(-c2ccc4ccc5cccc6ccc2c4c56)c13 ; c1ccc2cc3c(cc2c1)oc1cc(-c2c4ccccc4cc4c2ccc2ccccc24)cc(-c2ccc4ccc5cccc6ccc2c4c56)c13]

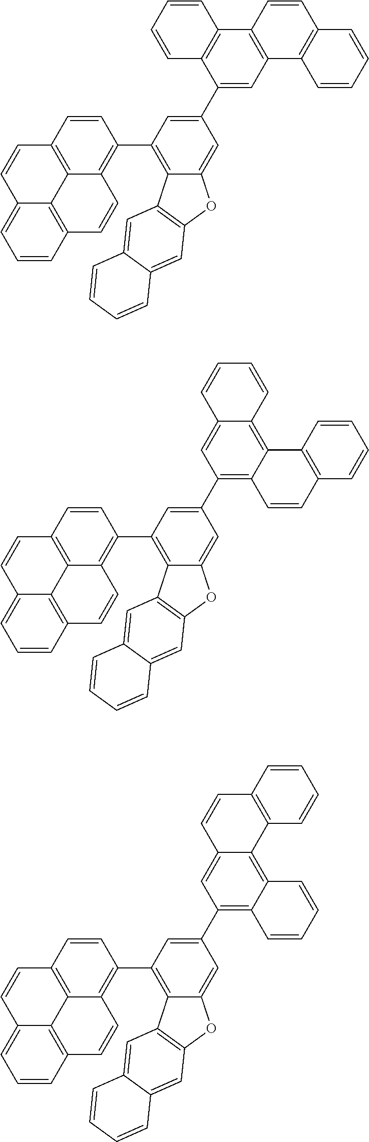 [SMILES: c1ccc2cc3c(cc2c1)oc1cc(-c2cc4c5ccccc5ccc4c4ccccc24)cc(-c2ccc4ccc5cccc6ccc2c4c56)c13 ; c1ccc2cc3c(cc2c1)oc1cc(-c2cc4ccccc4c4c2ccc2ccccc24)cc(-c2ccc4ccc5cccc6ccc2c4c56)c13 ; c1ccc2cc3c(cc2c1)oc1cc(-c2cc4ccc5ccccc5c4c4ccccc24)cc(-c2ccc4ccc5cccc6ccc2c4c56)c13]

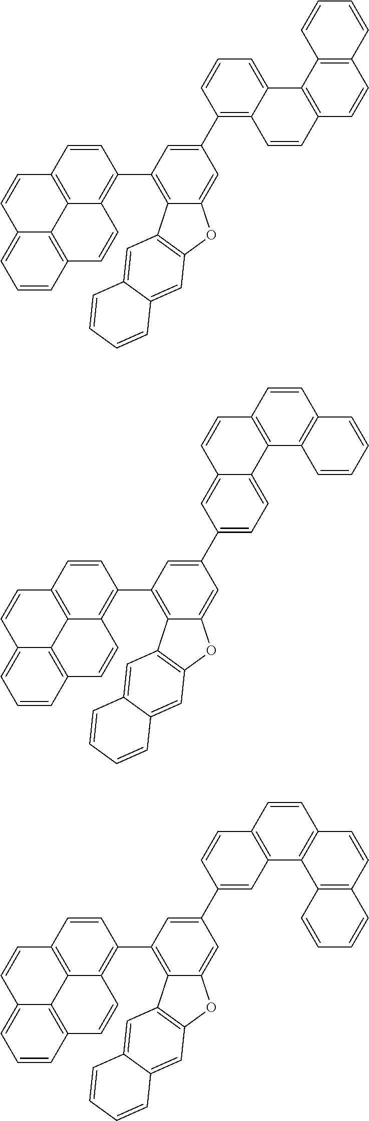 [SMILES: c1ccc2cc3c(cc2c1)oc1cc(-c2cccc4c2ccc2ccc5ccccc5c24)cc(-c2ccc4ccc5cccc6ccc2c4c56)c13 ; c1ccc2cc3c(cc2c1)oc1cc(-c2ccc4c(ccc5ccc6ccccc6c54)c2)cc(-c2ccc4ccc5cccc6ccc2c4c56)c13 ; c1ccc2cc3c(cc2c1)oc1cc(-c2ccc4ccc5ccc6ccccc6c5c4c2)cc(-c2ccc4ccc5cccc6ccc2c4c56)c13]

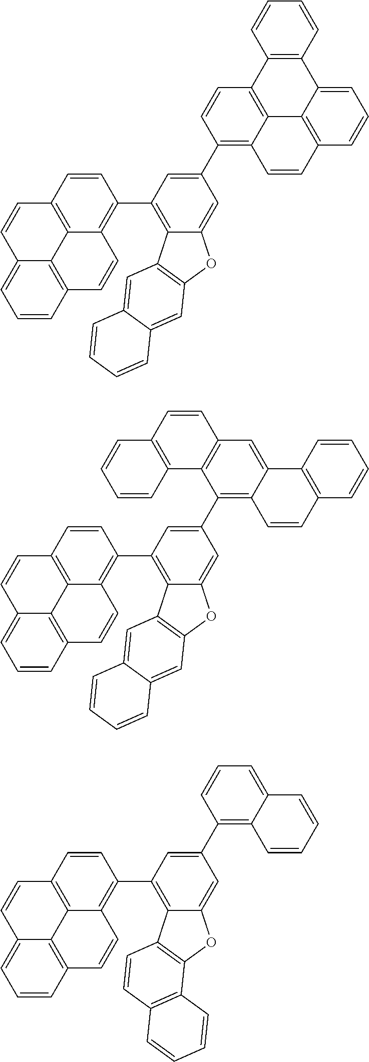 [SMILES: c1ccc2cc3c(cc2c1)oc1cc(-c2ccc4c5ccccc5c5cccc6ccc2c4c65)cc(-c2ccc4ccc5cccc6ccc2c4c56)c13 ; c1ccc2cc3c(cc2c1)oc1cc(-c2c4ccc5ccccc5c4cc4ccc5ccccc5c24)cc(-c2ccc4ccc5cccc6ccc2c4c56)c13 ; c1ccc2c(-c3cc(-c4ccc5ccc6cccc7ccc4c5c67)c4c(c3)oc3c5ccccc5ccc34)cccc2c1]

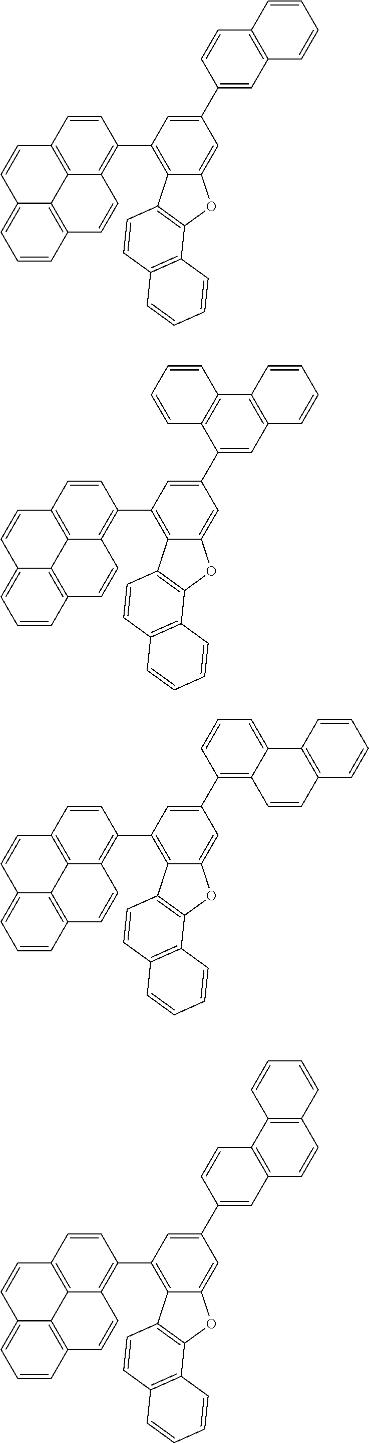

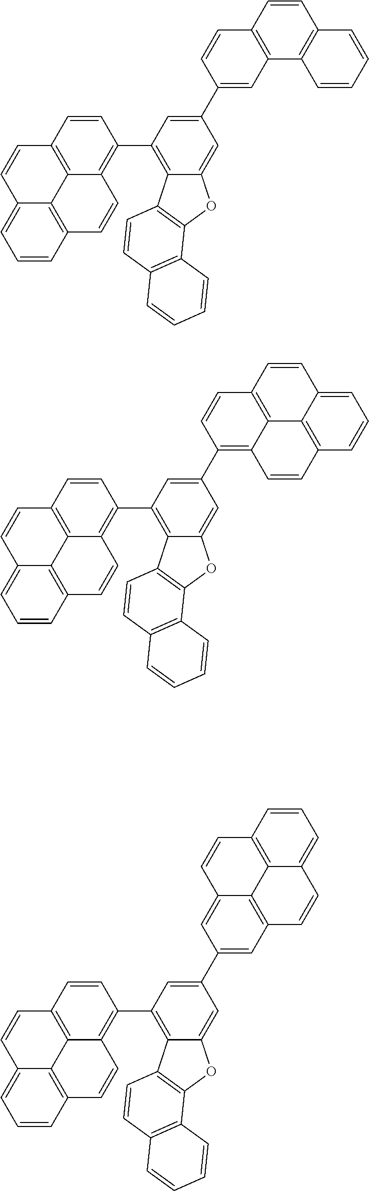 [SMILES: c1ccc2c(c1)ccc1ccc(-c3cc(-c4ccc5ccc6cccc7ccc4c5c67)c4c(c3)oc3c5ccccc5ccc34)cc12 ; c1ccc2c(c1)ccc1c2oc2cc(-c3ccc4ccc5cccc6ccc3c4c56)cc(-c3ccc4ccc5cccc6ccc3c4c56)c21 ; c1ccc2c(c1)ccc1c2oc2cc(-c3cc4ccc5cccc6ccc(c3)c4c56)cc(-c3ccc4ccc5cccc6ccc3c4c56)c21]

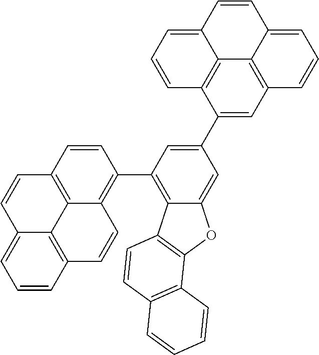 [SMILES: c1ccc2c(c1)ccc1c2oc2cc(-c3cc4cccc5ccc6cccc3c6c54)cc(-c3ccc4ccc5cccc6ccc3c4c56)c21]

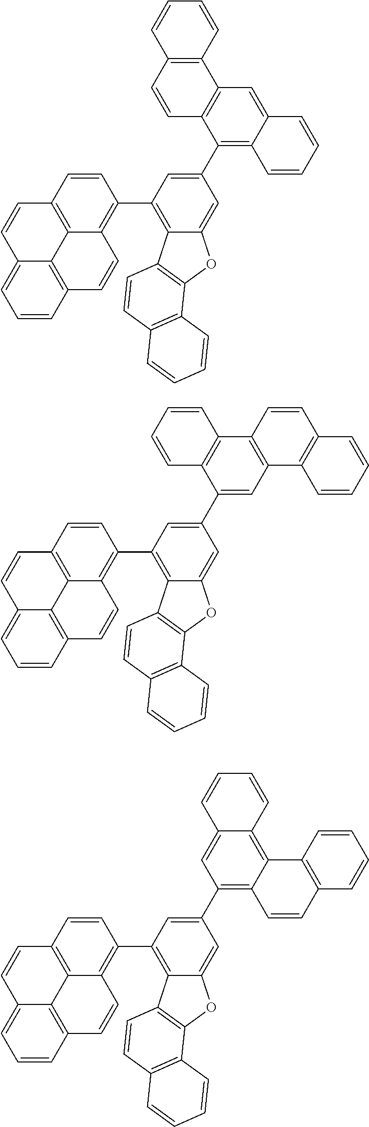

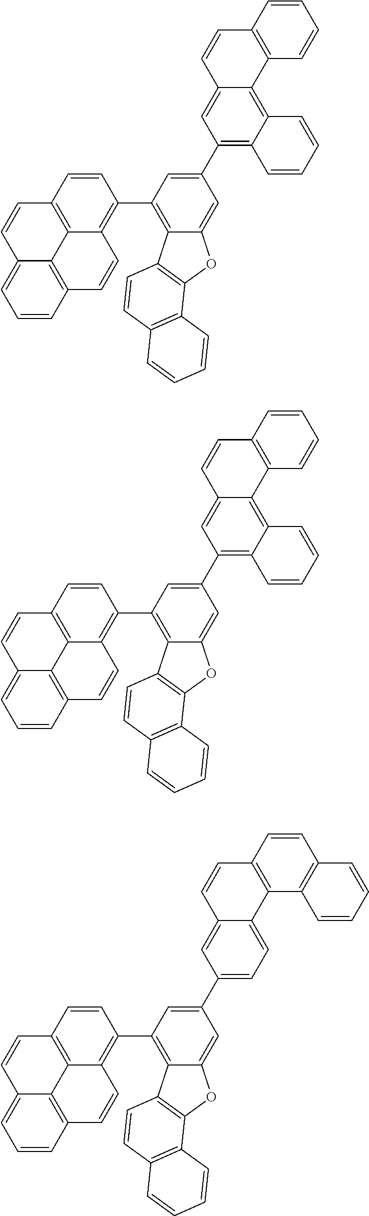 [SMILES: c1ccc2c(c1)ccc1c2oc2cc(-c3cc4ccc5ccccc5c4c4ccccc34)cc(-c3ccc4ccc5cccc6ccc3c4c56)c21 ; c1ccc2c(c1)ccc1c2oc2cc(-c3cc4ccc5ccccc5c4c4ccccc34)cc(-c3ccc4ccc5cccc6ccc3c4c56)c21 ; c1ccc2c(c1)ccc1c2oc2cc(-c3ccc4c(ccc5ccc6ccccc6c54)c3)cc(-c3ccc4ccc5cccc6ccc3c4c56)c21]

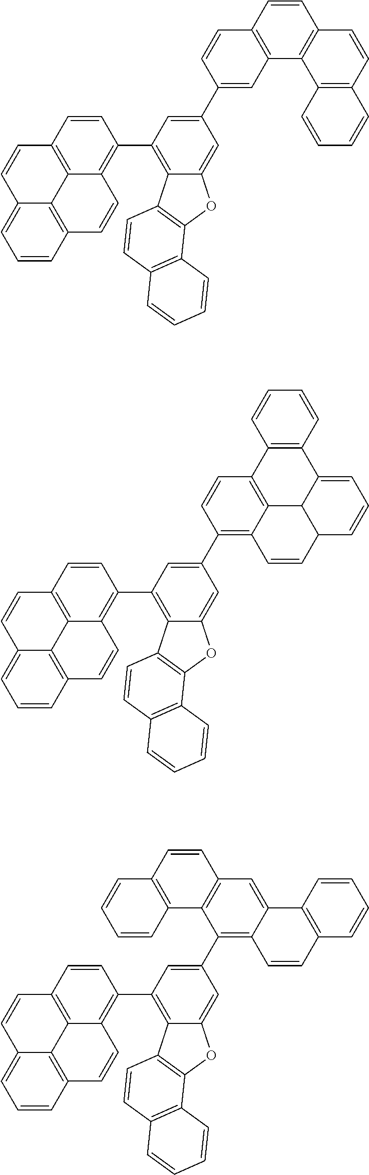 [SMILES: c1ccc2c(c1)ccc1c2oc2cc(-c3ccc4ccc5ccc6ccccc6c5c4c3)cc(-c3ccc4ccc5cccc6ccc3c4c56)c21 ; C1=CC2C=Cc3c(-c4cc(-c5ccc6ccc7cccc8ccc5c6c78)c5c(c4)oc4c6ccccc6ccc45)ccc4c3C2C(=C1)c1ccccc1-4 ; c1ccc2c(c1)ccc1c(-c3cc(-c4ccc5ccc6cccc7ccc4c5c67)c4c(c3)oc3c5ccccc5ccc34)c3c(ccc4ccccc43)cc12]

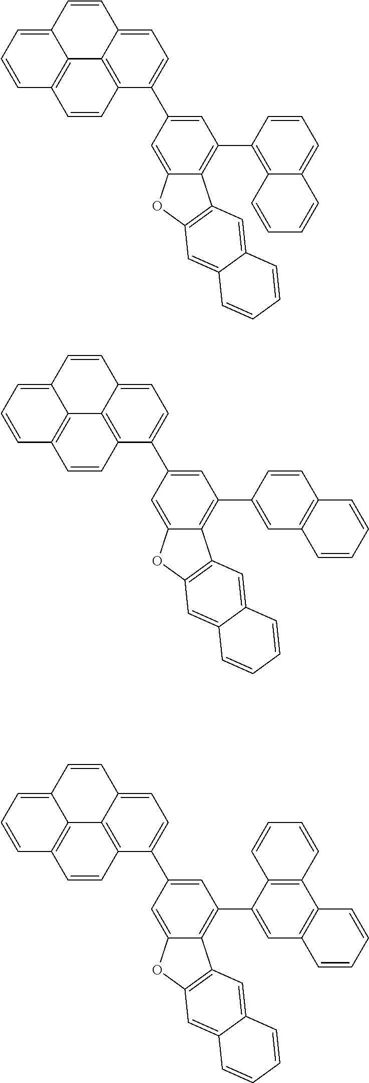 [SMILES: c1ccc2cc3c(cc2c1)oc1cc(-c2ccc4ccc5cccc6ccc2c4c56)cc(-c2cccc4ccccc24)c13 ; c1ccc2cc(-c3cc(-c4ccc5ccc6cccc7ccc4c5c67)cc4oc5cc6ccccc6cc5c34)ccc2c1 ; c1ccc2cc3c(cc2c1)oc1cc(-c2ccc4ccc5cccc6ccc2c4c56)cc(-c2cc4ccccc4c4ccccc24)c13]

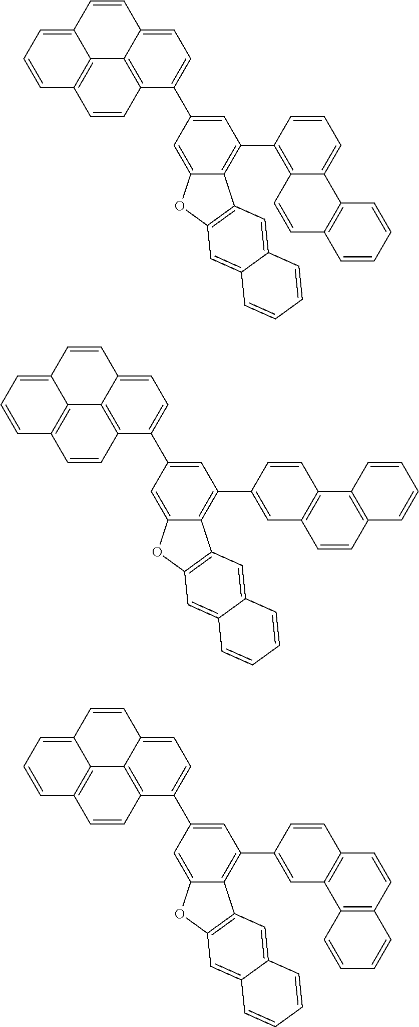 [SMILES: c1ccc2cc3c(cc2c1)oc1cc(-c2ccc4ccc5cccc6ccc2c4c56)cc(-c2cccc4c2ccc2ccccc24)c13 ; c1ccc2cc3c(cc2c1)oc1cc(-c2ccc4ccc5cccc6ccc2c4c56)cc(-c2ccc4c(ccc5ccccc54)c2)c13 ; c1ccc2cc3c(cc2c1)oc1cc(-c2ccc4ccc5cccc6ccc2c4c56)cc(-c2ccc4ccc5ccccc5c4c2)c13]

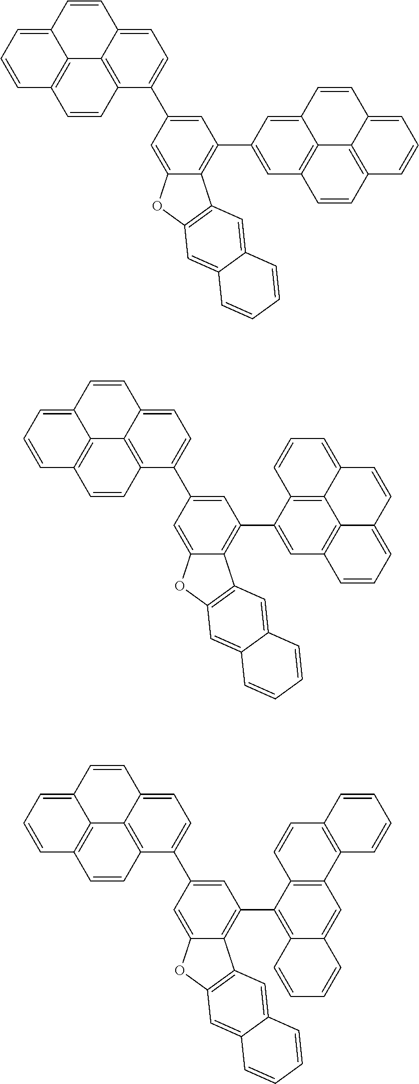 [SMILES: c1ccc2cc3c(cc2c1)oc1cc(-c2ccc4ccc5cccc6ccc2c4c56)cc(-c2cc4ccc5cccc6ccc(c2)c4c56)c13 ; c1ccc2cc3c(cc2c1)oc1cc(-c2ccc4ccc5cccc6ccc2c4c56)cc(-c2cc4cccc5ccc6cccc2c6c54)c13 ; c1ccc2cc3c(cc2c1)oc1cc(-c2ccc4ccc5cccc6ccc2c4c56)cc(-c2c4ccccc4cc4c2ccc2ccccc24)c13]

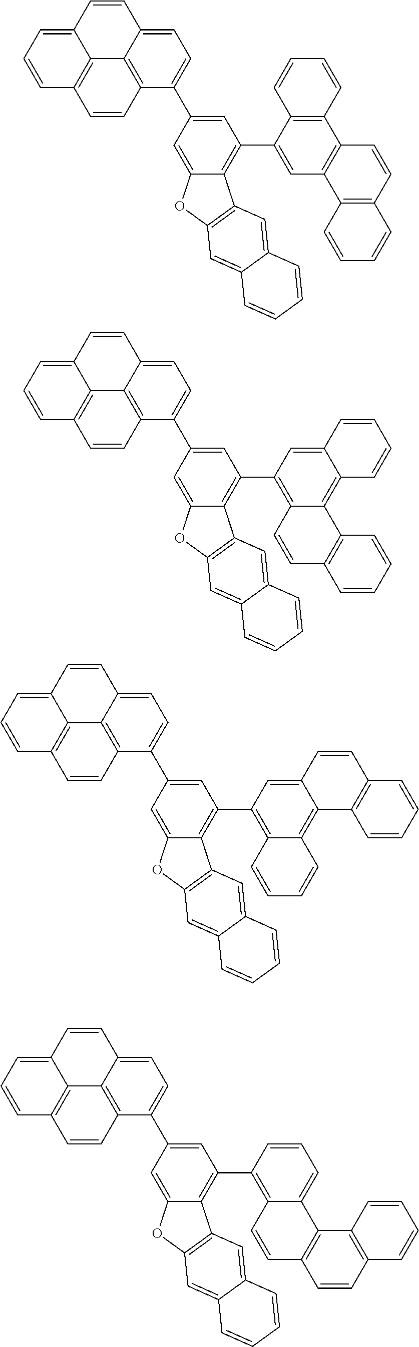 [SMILES: c1ccc2cc3c(cc2c1)oc1cc(-c2ccc4ccc5cccc6ccc2c4c56)cc(-c2cc4c5ccccc5ccc4c4ccccc24)c13 ; c1ccc2cc3c(cc2c1)oc1cc(-c2ccc4ccc5cccc6ccc2c4c56)cc(-c2cc4ccccc4c4c2ccc2ccccc24)c13 ; c1ccc2cc3c(cc2c1)oc1cc(-c2ccc4ccc5cccc6ccc2c4c56)cc(-c2cc4ccc5ccccc5c4c4ccccc24)c13 ; c1ccc2cc3c(cc2c1)oc1cc(-c2ccc4ccc5cccc6ccc2c4c56)cc(-c2cccc4c2ccc2ccc5ccccc5c24)c13]

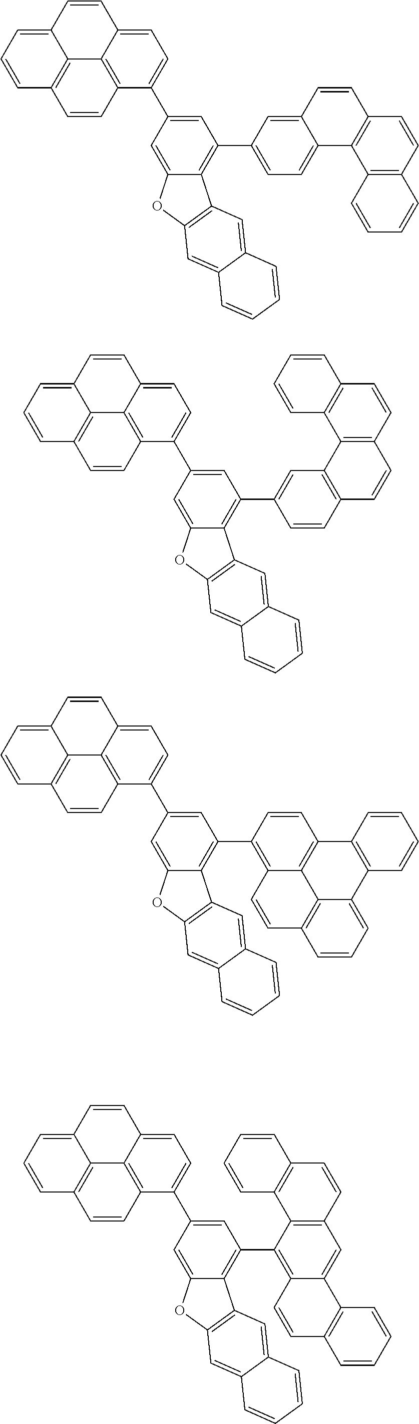 [SMILES: c1ccc2cc3c(cc2c1)oc1cc(-c2ccc4ccc5cccc6ccc2c4c56)cc(-c2ccc4c(ccc5ccc6ccccc6c54)c2)c13 ; c1ccc2cc3c(cc2c1)oc1cc(-c2ccc4ccc5cccc6ccc2c4c56)cc(-c2ccc4ccc5ccc6ccccc6c5c4c2)c13 ; c1ccc2cc3c(cc2c1)oc1cc(-c2ccc4ccc5cccc6ccc2c4c56)cc(-c2ccc4c5ccccc5c5cccc6ccc2c4c65)c13 ; c1ccc2cc3c(cc2c1)oc1cc(-c2ccc4ccc5cccc6ccc2c4c56)cc(-c2c4ccc5ccccc5c4cc4ccc5ccccc5c24)c13]

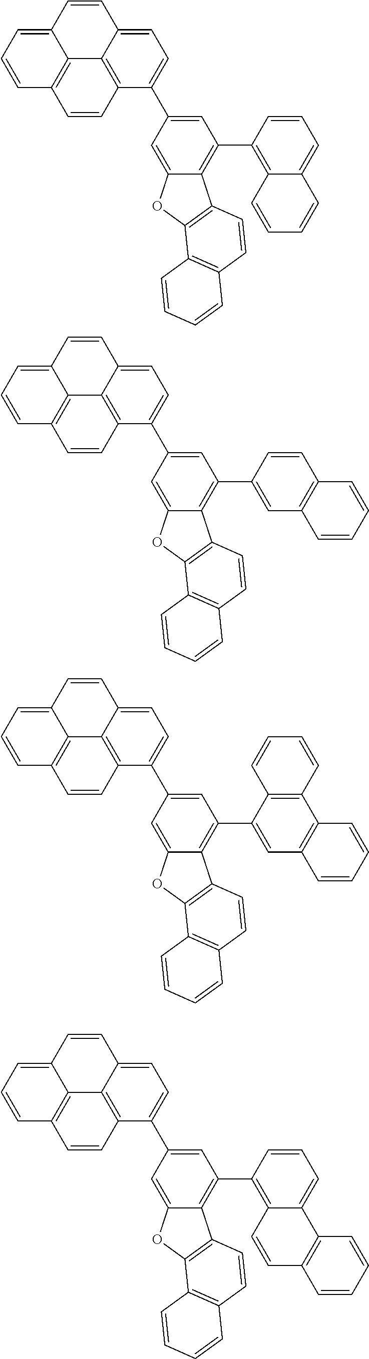 [SMILES: c1ccc2c(-c3cc(-c4ccc5ccc6cccc7ccc4c5c67)cc4oc5c6ccccc6ccc5c34)cccc2c1 ; c1ccc2cc(-c3cc(-c4ccc5ccc6cccc7ccc4c5c67)cc4oc5c6ccccc6ccc5c34)ccc2c1 ; c1ccc2c(c1)cc(-c1cc(-c3ccc4ccc5cccc6ccc3c4c56)cc3oc4c5ccccc5ccc4c13)c1ccccc12 ; c1ccc2c(c1)ccc1c(-c3cc(-c4ccc5ccc6cccc7ccc4c5c67)cc4oc5c6ccccc6ccc5c34)cccc12]

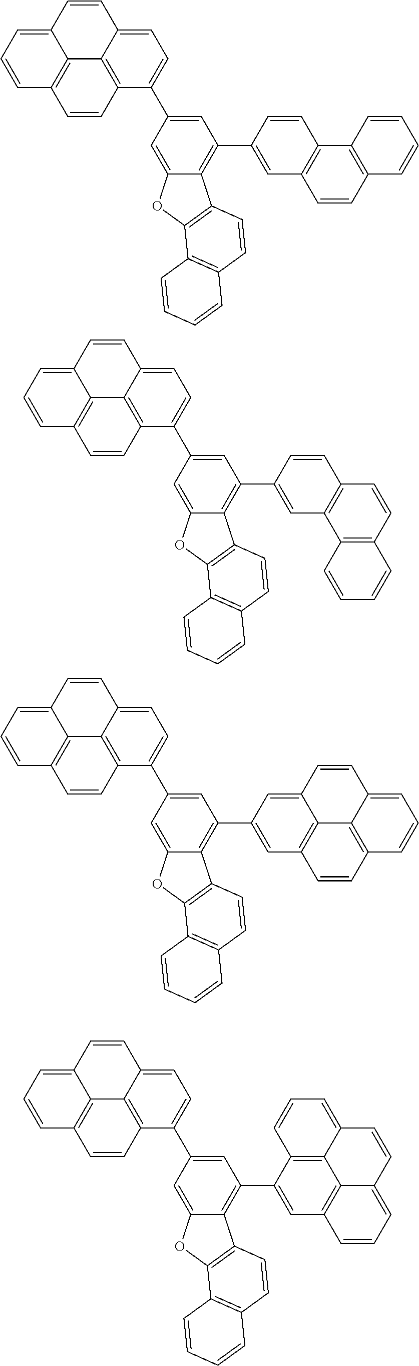 [SMILES: c1ccc2c(c1)ccc1cc(-c3cc(-c4ccc5ccc6cccc7ccc4c5c67)cc4oc5c6ccccc6ccc5c34)ccc12 ; c1ccc2c(c1)ccc1ccc(-c3cc(-c4ccc5ccc6cccc7ccc4c5c67)cc4oc5c6ccccc6ccc5c34)cc12 ; c1ccc2c(c1)ccc1c2oc2cc(-c3ccc4ccc5cccc6ccc3c4c56)cc(-c3cc4ccc5cccc6ccc(c3)c4c56)c21 ; c1ccc2c(c1)ccc1c2oc2cc(-c3ccc4ccc5cccc6ccc3c4c56)cc(-c3cc4cccc5ccc6cccc3c6c54)c21]

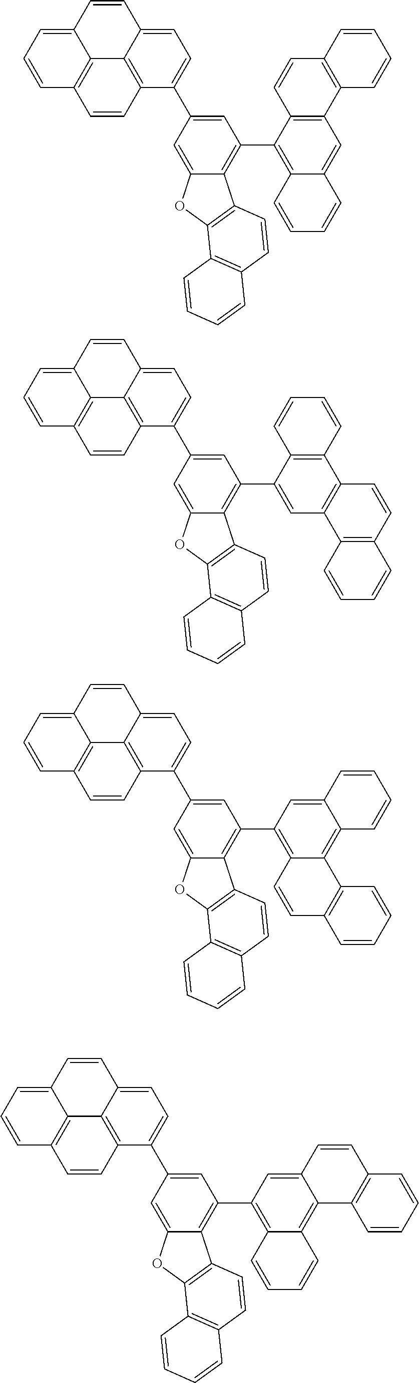 [SMILES: c1ccc2c(-c3cc(-c4ccc5ccc6cccc7ccc4c5c67)cc4oc5c6ccccc6ccc5c34)c3ccc4ccccc4c3cc2c1 ; c1ccc2c(c1)ccc1c3ccccc3c(-c3cc(-c4ccc5ccc6cccc7ccc4c5c67)cc4oc5c6ccccc6ccc5c34)cc21 ; c1ccc2c(c1)ccc1c2oc2cc(-c3ccc4ccc5cccc6ccc3c4c56)cc(-c3cc4ccccc4c4c3ccc3ccccc34)c21 ; c1ccc2c(c1)ccc1c2oc2cc(-c3ccc4ccc5cccc6ccc3c4c56)cc(-c3cc4ccc5ccccc5c4c4ccccc34)c21]

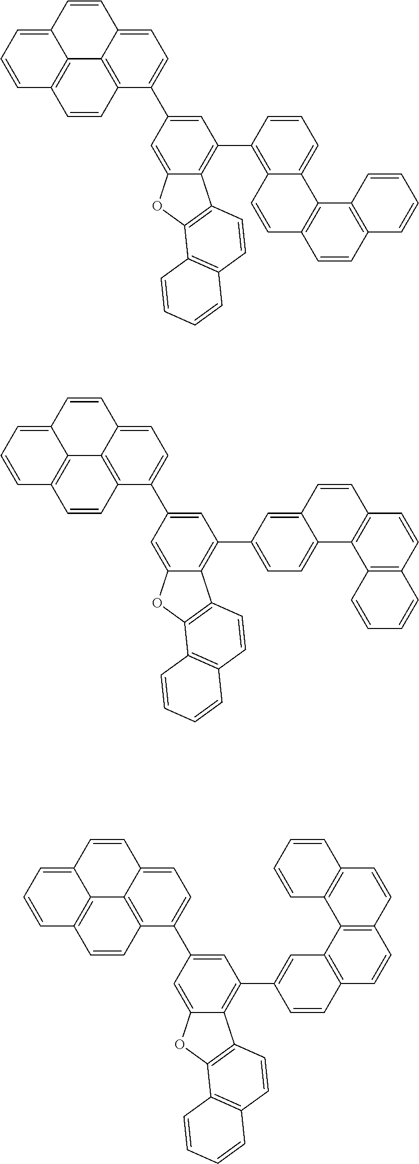 [SMILES: c1ccc2c(c1)ccc1c2oc2cc(-c3ccc4ccc5cccc6ccc3c4c56)cc(-c3cccc4c3ccc3ccc5ccccc5c34)c21 ; c1ccc2c(c1)ccc1c2oc2cc(-c3ccc4ccc5cccc6ccc3c4c56)cc(-c3ccc4c(ccc5ccc6ccccc6c54)c3)c21 ; c1ccc2c(c1)ccc1c2oc2cc(-c3ccc4ccc5cccc6ccc3c4c56)cc(-c3ccc4ccc5ccc6ccccc6c5c4c3)c21]

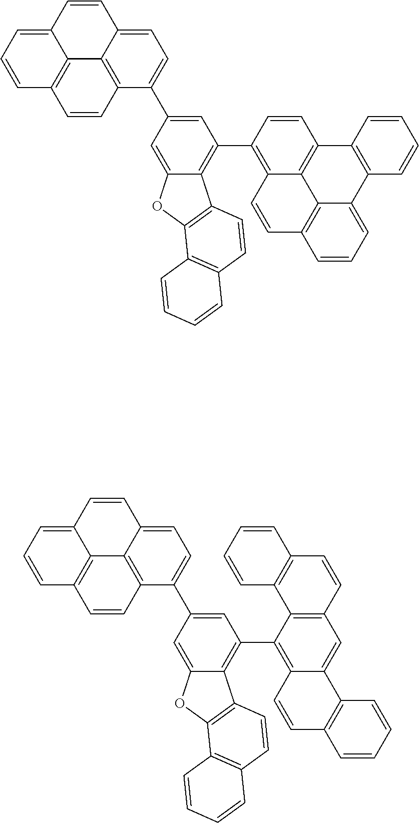 [SMILES: c1ccc2c(c1)ccc1c2oc2cc(-c3ccc4ccc5cccc6ccc3c4c56)cc(-c3ccc4c5ccccc5c5cccc6ccc3c4c65)c21 ; c1ccc2c(c1)ccc1c(-c3cc(-c4ccc5ccc6cccc7ccc4c5c67)cc4oc5c6ccccc6ccc5c34)c3c(ccc4ccccc43)cc12]

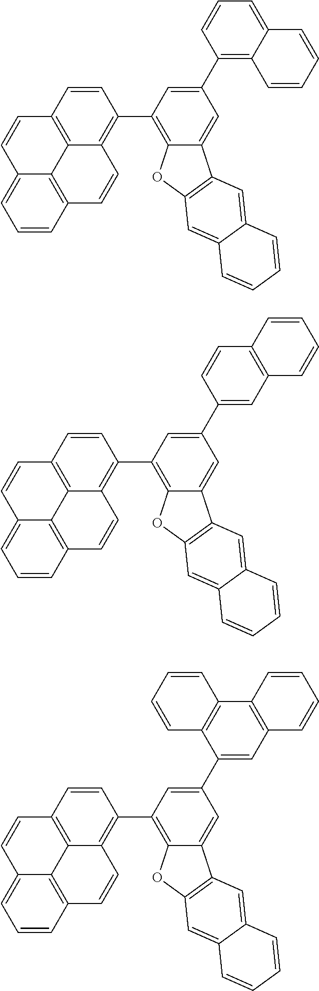

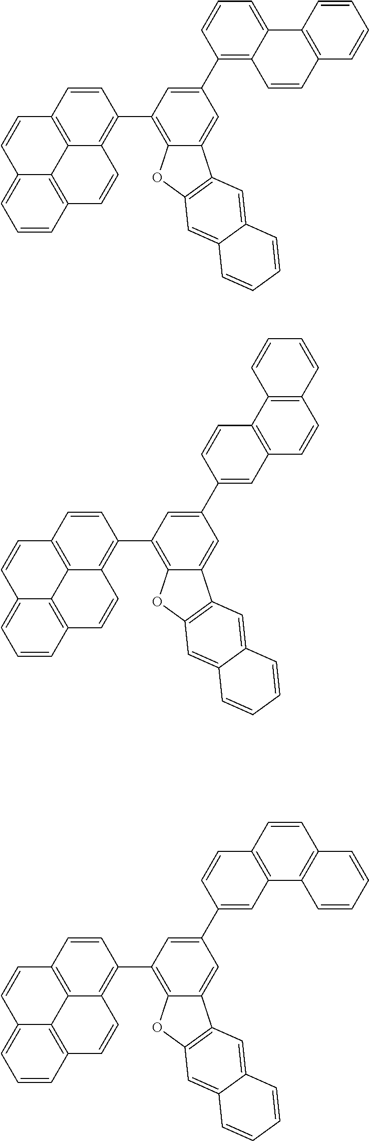 [SMILES: c1ccc2cc3c(cc2c1)oc1c(-c2ccc4ccc5cccc6ccc2c4c56)cc(-c2cccc4c2ccc2ccccc24)cc13 ; c1ccc2cc3c(cc2c1)oc1c(-c2ccc4ccc5cccc6ccc2c4c56)cc(-c2ccc4c(ccc5ccccc54)c2)cc13 ; c1ccc2cc3c(cc2c1)oc1c(-c2ccc4ccc5cccc6ccc2c4c56)cc(-c2ccc4ccc5ccccc5c4c2)cc13]

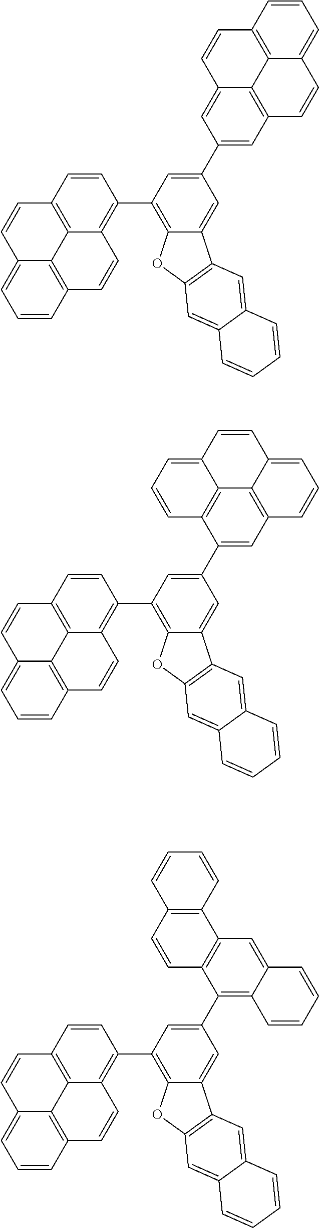 [SMILES: c1ccc2cc3c(cc2c1)oc1c(-c2ccc4ccc5cccc6ccc2c4c56)cc(-c2cc4ccc5cccc6ccc(c2)c4c56)cc13 ; c1ccc2cc3c(cc2c1)oc1c(-c2ccc4ccc5cccc6ccc2c4c56)cc(-c2cc4cccc5ccc6cccc2c6c54)cc13 ; c1ccc2cc3c(cc2c1)oc1c(-c2ccc4ccc5cccc6ccc2c4c56)cc(-c2c4ccccc4cc4c2ccc2ccccc24)cc13]

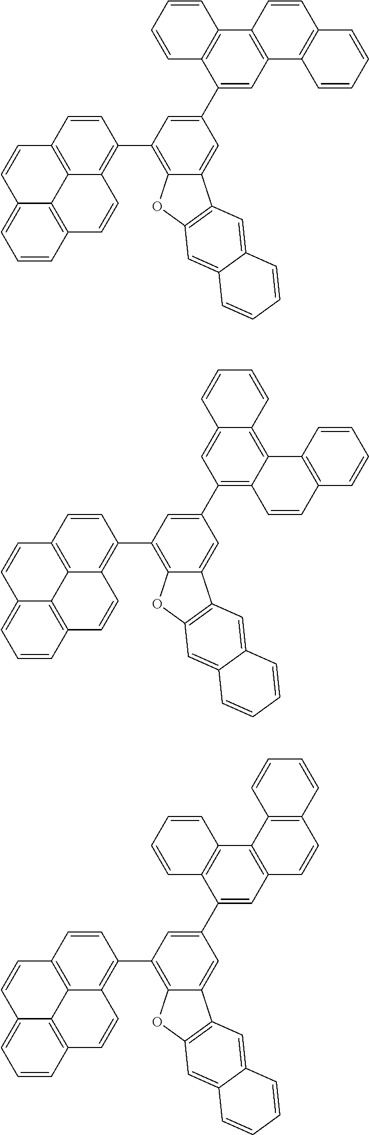

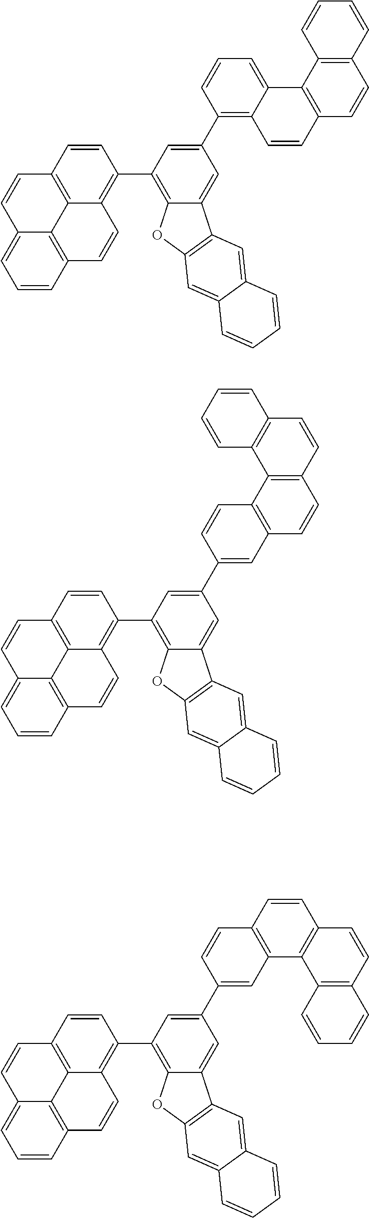

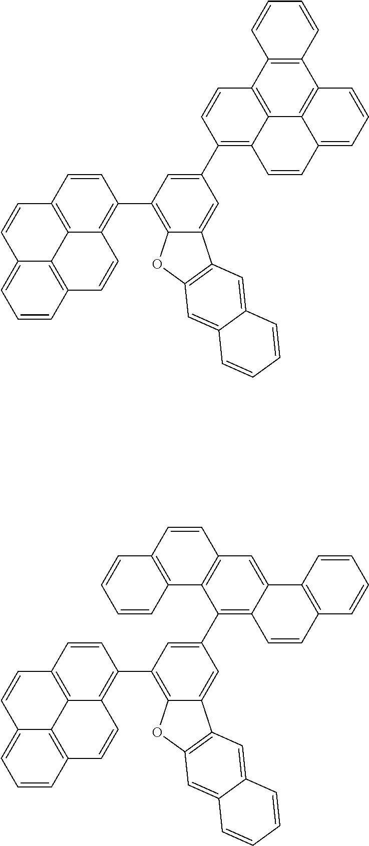 [SMILES: c1ccc2cc3c(cc2c1)oc1c(-c2ccc4ccc5cccc6ccc2c4c56)cc(-c2ccc4c5ccccc5c5cccc6ccc2c4c65)cc13 ; c1ccc2cc3c(cc2c1)oc1c(-c2ccc4ccc5cccc6ccc2c4c56)cc(-c2c4ccc5ccccc5c4cc4ccc5ccccc5c24)cc13]

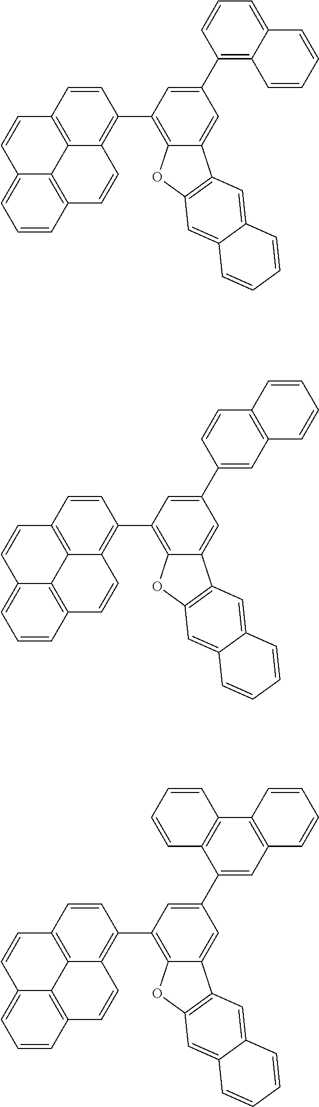 [SMILES: c1ccc2cc3c(cc2c1)oc1c(-c2ccc4ccc5cccc6ccc2c4c56)cc(-c2cccc4ccccc24)cc13 ; c1ccc2cc(-c3cc(-c4ccc5ccc6cccc7ccc4c5c67)c4oc5cc6ccccc6cc5c4c3)ccc2c1 ; c1ccc2cc3c(cc2c1)oc1c(-c2ccc4ccc5cccc6ccc2c4c56)cc(-c2cc4ccccc4c4ccccc24)cc13]

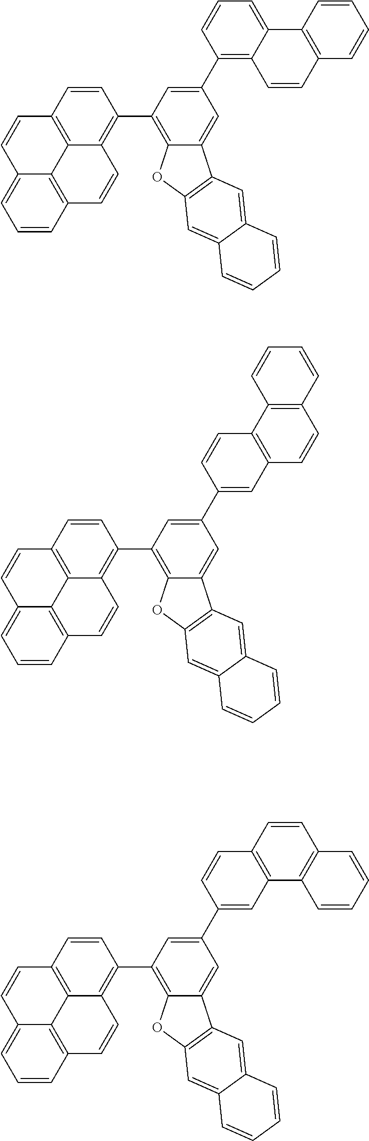 [SMILES: c1ccc2cc3c(cc2c1)oc1c(-c2ccc4ccc5cccc6ccc2c4c56)cc(-c2cccc4c2ccc2ccccc24)cc13 ; c1ccc2cc3c(cc2c1)oc1c(-c2ccc4ccc5cccc6ccc2c4c56)cc(-c2ccc4c(ccc5ccccc54)c2)cc13 ; c1ccc2cc3c(cc2c1)oc1c(-c2ccc4ccc5cccc6ccc2c4c56)cc(-c2ccc4ccc5ccccc5c4c2)cc13]

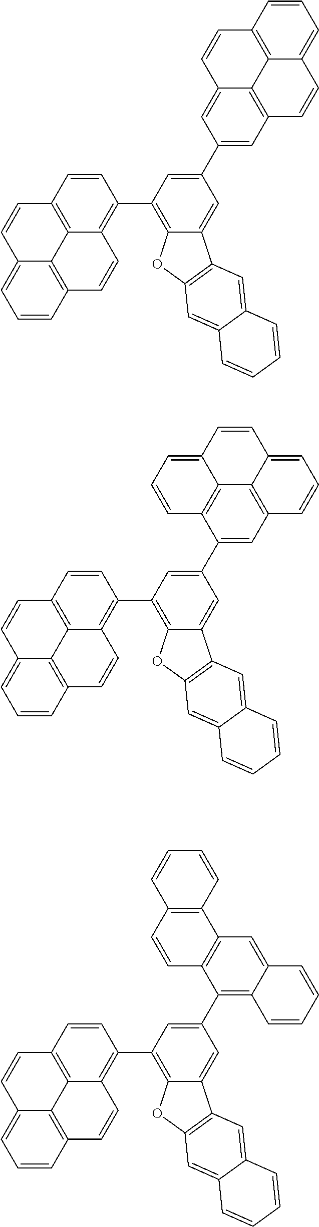 [SMILES: c1ccc2cc3c(cc2c1)oc1c(-c2ccc4ccc5cccc6ccc2c4c56)cc(-c2cc4ccc5cccc6ccc(c2)c4c56)cc13 ; c1ccc2cc3c(cc2c1)oc1c(-c2ccc4ccc5cccc6ccc2c4c56)cc(-c2cc4cccc5ccc6cccc2c6c54)cc13 ; c1ccc2cc3c(cc2c1)oc1c(-c2ccc4ccc5cccc6ccc2c4c56)cc(-c2c4ccccc4cc4c2ccc2ccccc24)cc13]

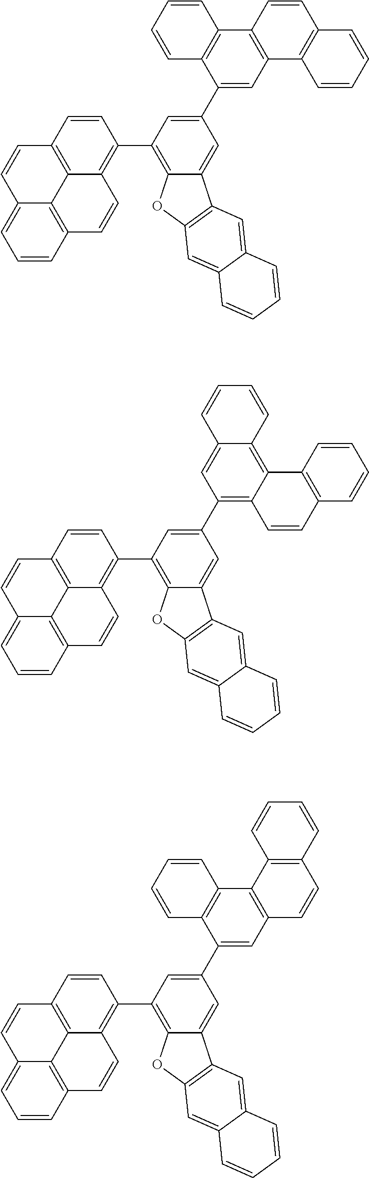 [SMILES: c1ccc2cc3c(cc2c1)oc1c(-c2ccc4ccc5cccc6ccc2c4c56)cc(-c2cc4c5ccccc5ccc4c4ccccc24)cc13 ; c1ccc2cc3c(cc2c1)oc1c(-c2ccc4ccc5cccc6ccc2c4c56)cc(-c2cc4ccccc4c4c2ccc2ccccc24)cc13 ; c1ccc2cc3c(cc2c1)oc1c(-c2ccc4ccc5cccc6ccc2c4c56)cc(-c2cc4ccc5ccccc5c4c4ccccc24)cc13]

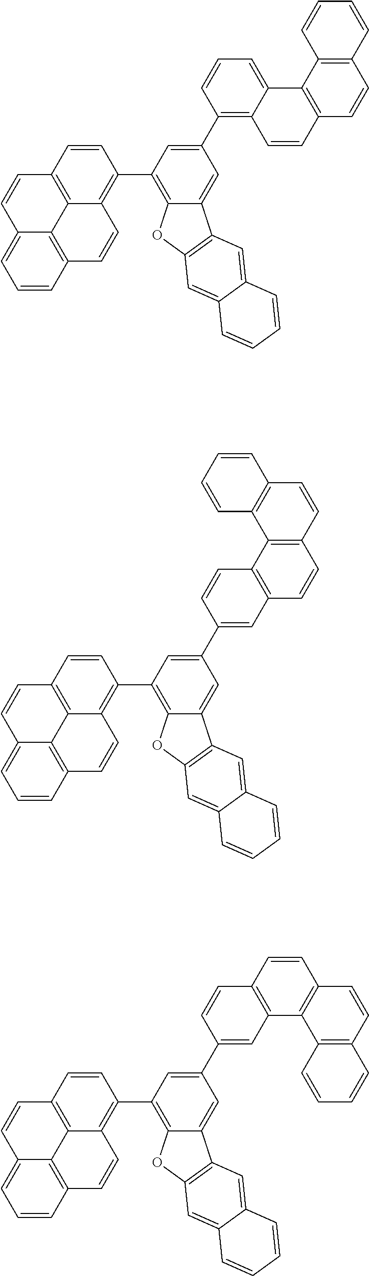 [SMILES: c1ccc2cc3c(cc2c1)oc1c(-c2ccc4ccc5cccc6ccc2c4c56)cc(-c2cccc4c2ccc2ccc5ccccc5c24)cc13 ; c1ccc2cc3c(cc2c1)oc1c(-c2ccc4ccc5cccc6ccc2c4c56)cc(-c2ccc4c(ccc5ccc6ccccc6c54)c2)cc13 ; c1ccc2cc3c(cc2c1)oc1c(-c2ccc4ccc5cccc6ccc2c4c56)cc(-c2ccc4ccc5ccc6ccccc6c5c4c2)cc13]

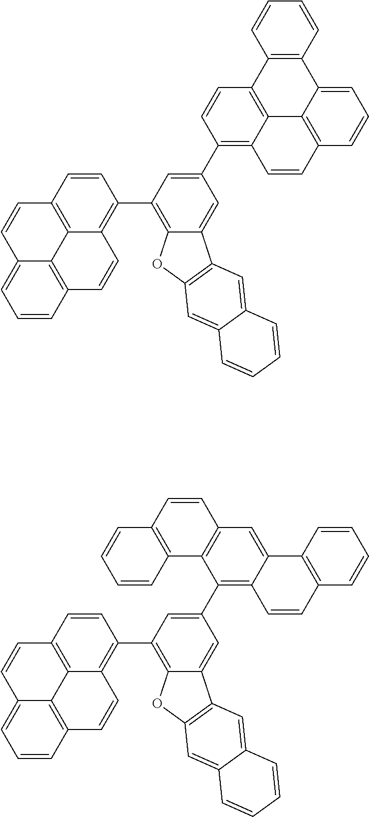 [SMILES: c1ccc2cc3c(cc2c1)oc1c(-c2ccc4ccc5cccc6ccc2c4c56)cc(-c2ccc4c5ccccc5c5cccc6ccc2c4c65)cc13 ; c1ccc2cc3c(cc2c1)oc1c(-c2ccc4ccc5cccc6ccc2c4c56)cc(-c2c4ccc5ccccc5c4cc4ccc5ccccc5c24)cc13]

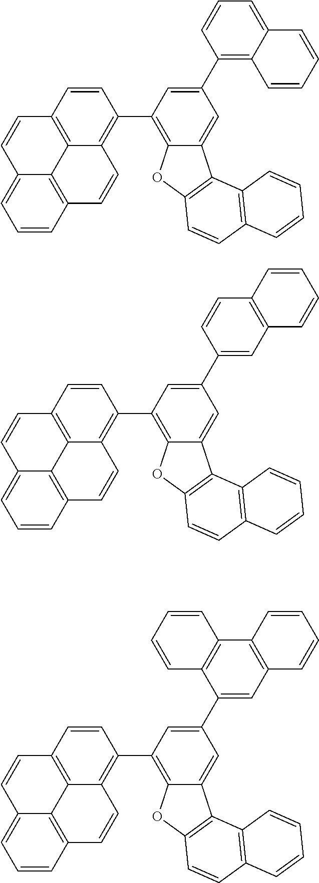

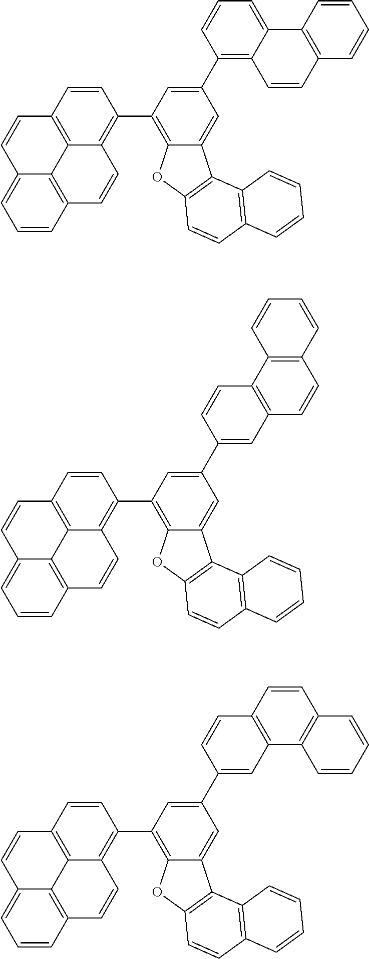 [SMILES: c1ccc2c(c1)ccc1c(-c3cc(-c4ccc5ccc6cccc7ccc4c5c67)c4oc5ccc6ccccc6c5c4c3)cccc12 ; c1ccc2c(c1)ccc1cc(-c3cc(-c4ccc5ccc6cccc7ccc4c5c67)c4oc5ccc6ccccc6c5c4c3)ccc12 ; c1ccc2c(c1)ccc1ccc(-c3cc(-c4ccc5ccc6cccc7ccc4c5c67)c4oc5ccc6ccccc6c5c4c3)cc12]

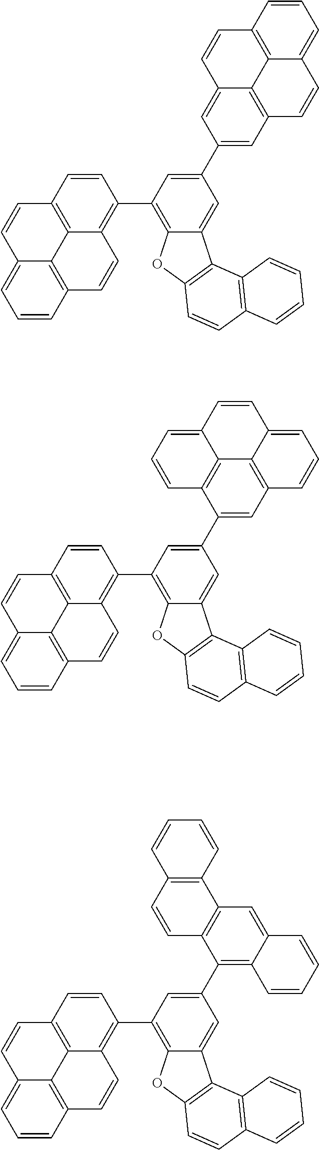 [SMILES: c1ccc2c(c1)ccc1oc3c(-c4ccc5ccc6cccc7ccc4c5c67)cc(-c4cc5ccc6cccc7ccc(c4)c5c67)cc3c12 ; c1ccc2c(c1)ccc1oc3c(-c4ccc5ccc6cccc7ccc4c5c67)cc(-c4cc5cccc6ccc7cccc4c7c65)cc3c12 ; c1ccc2c(-c3cc(-c4ccc5ccc6cccc7ccc4c5c67)c4oc5ccc6ccccc6c5c4c3)c3ccc4ccccc4c3cc2c1]

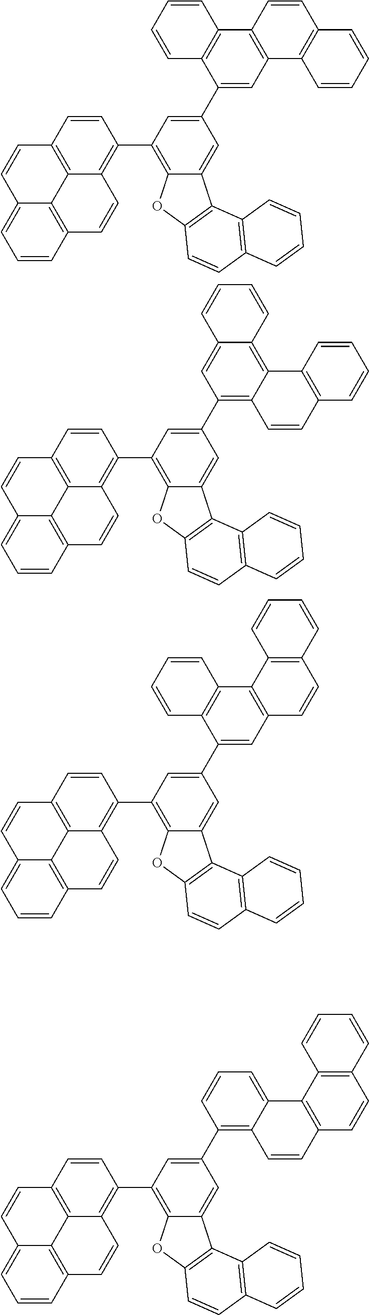

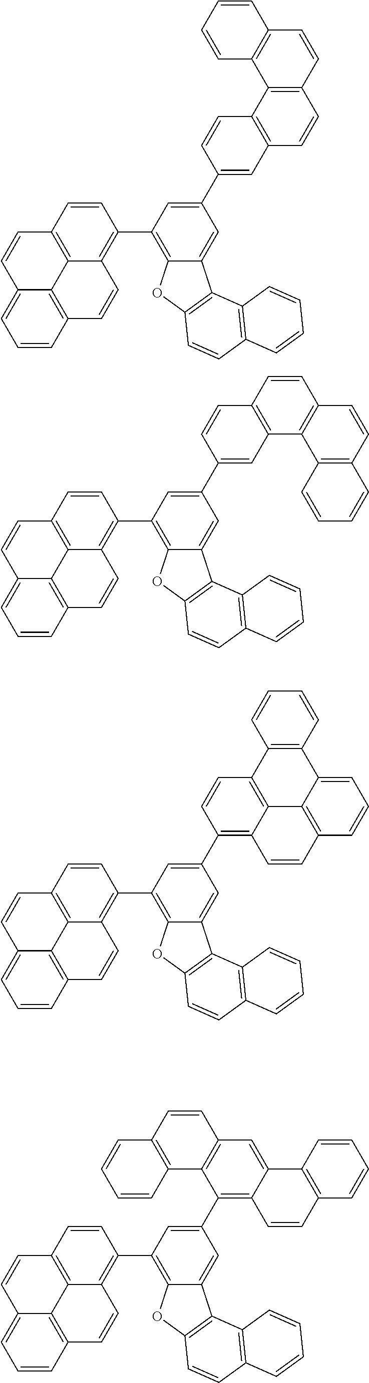

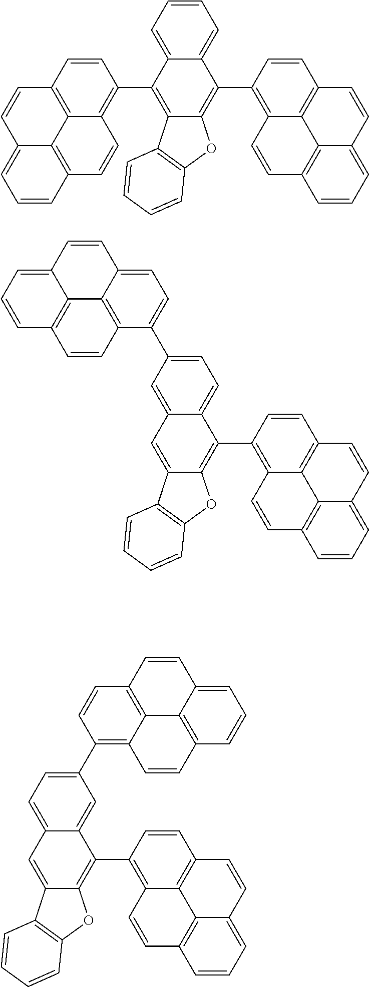

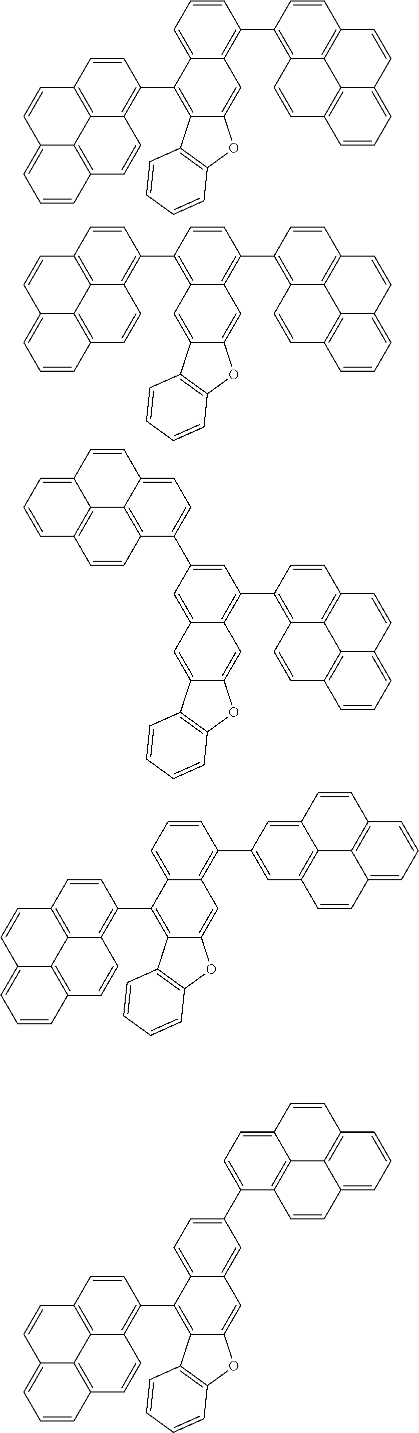

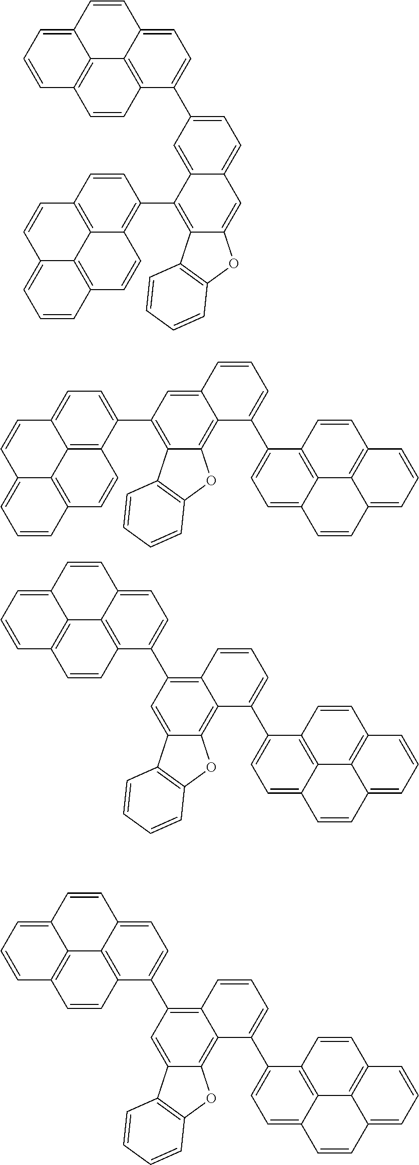

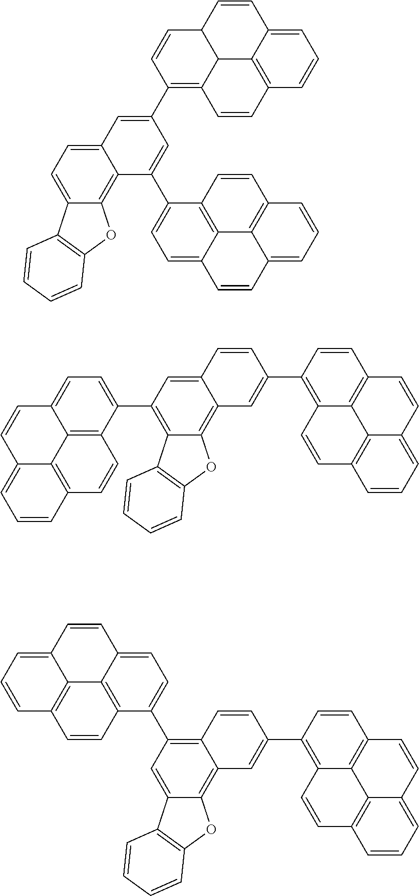 [SMILES: C1=CC2C=Cc3cccc4c3C2C(=C1c1cc(-c2ccc3ccc5cccc6ccc2c3c56)c2c(ccc3c5ccccc5oc32)c1)C=C4 ; c1cc2ccc3ccc(-c4ccc5cc(-c6ccc7ccc8cccc9ccc6c7c89)c6c7ccccc7oc6c5c4)c4ccc(c1)c2c34 ; c1cc2ccc3ccc(-c4ccc5c(-c6ccc7ccc8cccc9ccc6c7c89)cc6c7ccccc7oc6c5c4)c4ccc(c1)c2c34]

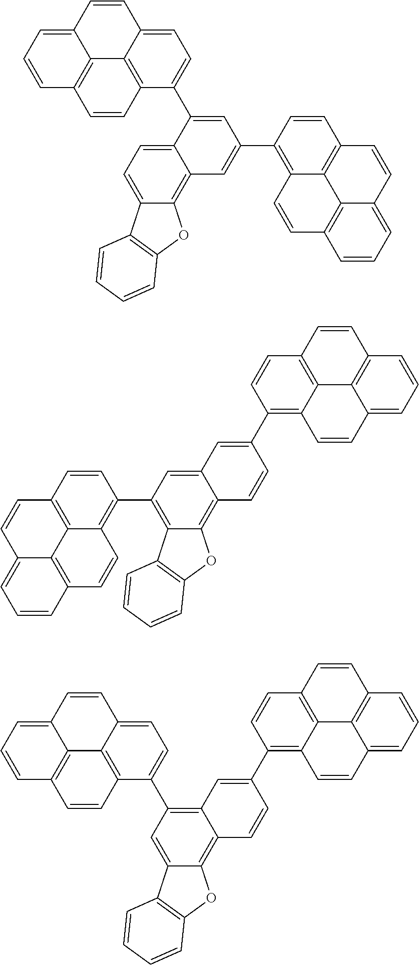 [SMILES: c1cc2ccc3ccc(-c4cc(-c5ccc6ccc7cccc8ccc5c6c78)c5ccc6c7ccccc7oc6c5c4)c4ccc(c1)c2c34 ; c1cc2ccc3ccc(-c4ccc5c(c4)cc(-c4ccc6ccc7cccc8ccc4c6c78)c4c6ccccc6oc54)c4ccc(c1)c2c34 ; c1cc2ccc3ccc(-c4ccc5c(c4)c(-c4ccc6ccc7cccc8ccc4c6c78)cc4c6ccccc6oc54)c4ccc(c1)c2c34]

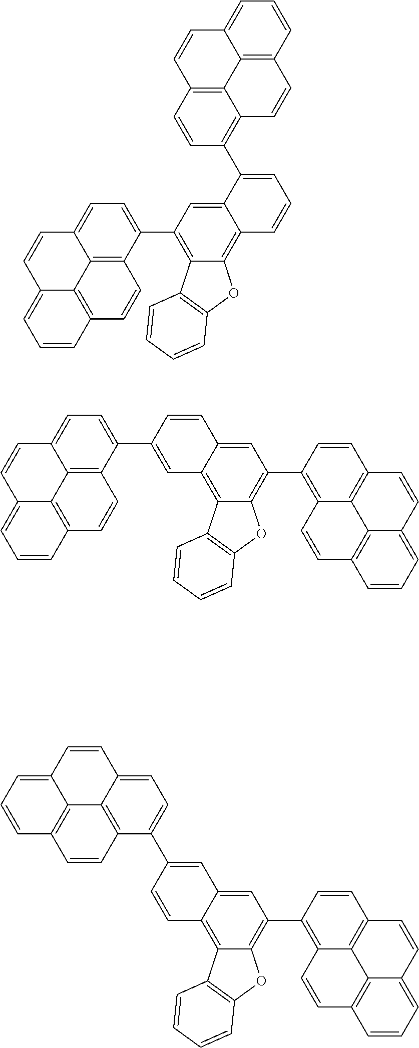 [SMILES: c1cc2ccc3ccc(-c4cccc5c4cc(-c4ccc6ccc7cccc8ccc4c6c78)c4c6ccccc6oc54)c4ccc(c1)c2c34 ; c1cc2ccc3ccc(-c4ccc5cc(-c6ccc7ccc8cccc9ccc6c7c89)c6oc7ccccc7c6c5c4)c4ccc(c1)c2c34 ; c1cc2ccc3ccc(-c4ccc5c(c4)cc(-c4ccc6ccc7cccc8ccc4c6c78)c4oc6ccccc6c45)c4ccc(c1)c2c34]

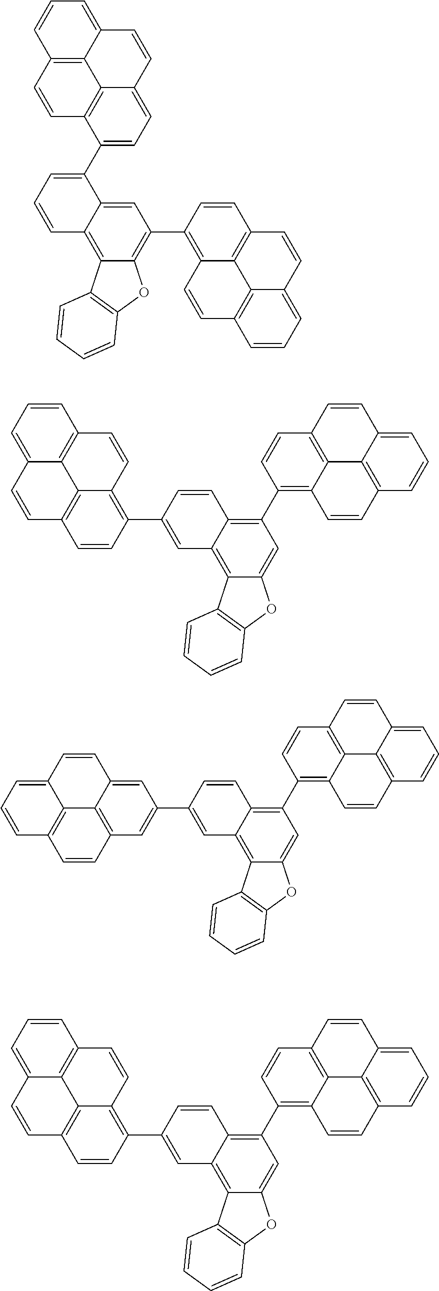 [SMILES: c1cc2ccc3ccc(-c4cccc5c4cc(-c4ccc6ccc7cccc8ccc4c6c78)c4oc6ccccc6c45)c4ccc(c1)c2c34 ; c1cc2ccc3ccc(-c4ccc5c(-c6ccc7ccc8cccc9ccc6c7c89)cc6oc7ccccc7c6c5c4)c4ccc(c1)c2c34 ; c1cc2ccc3cc(-c4ccc5c(-c6ccc7ccc8cccc9ccc6c7c89)cc6oc7ccccc7c6c5c4)cc4ccc(c1)c2c34 ; c1cc2ccc3ccc(-c4ccc5c(-c6ccc7ccc8cccc9ccc6c7c89)cc6oc7ccccc7c6c5c4)c4ccc(c1)c2c34]

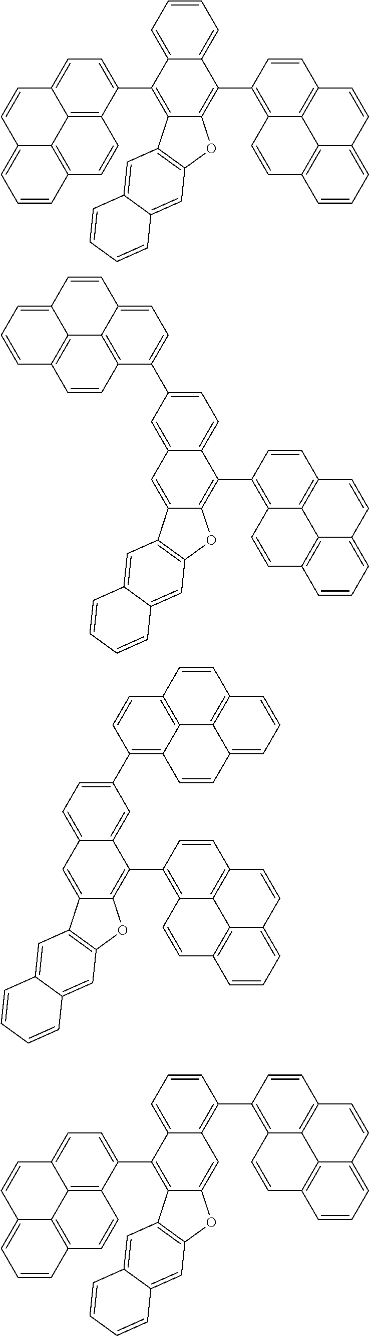

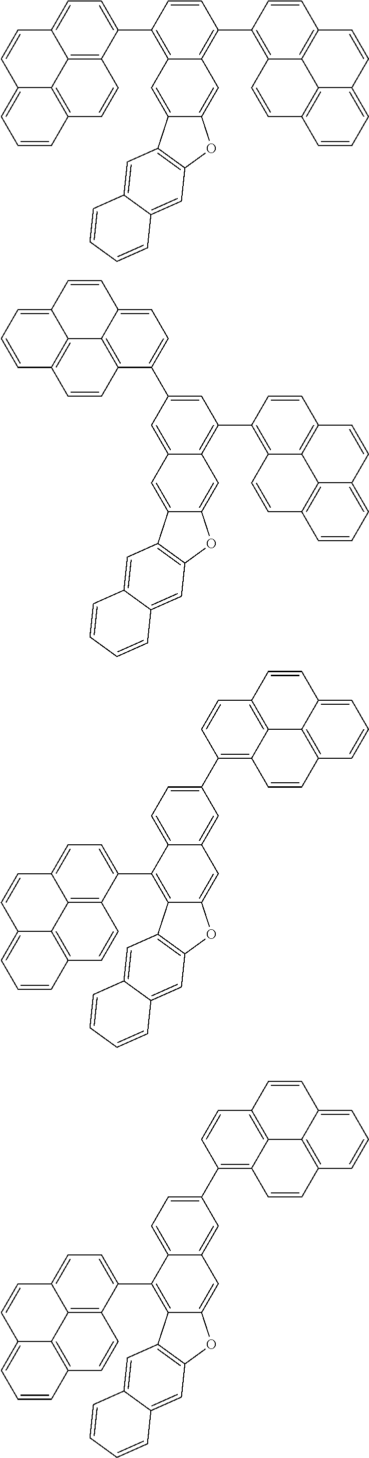

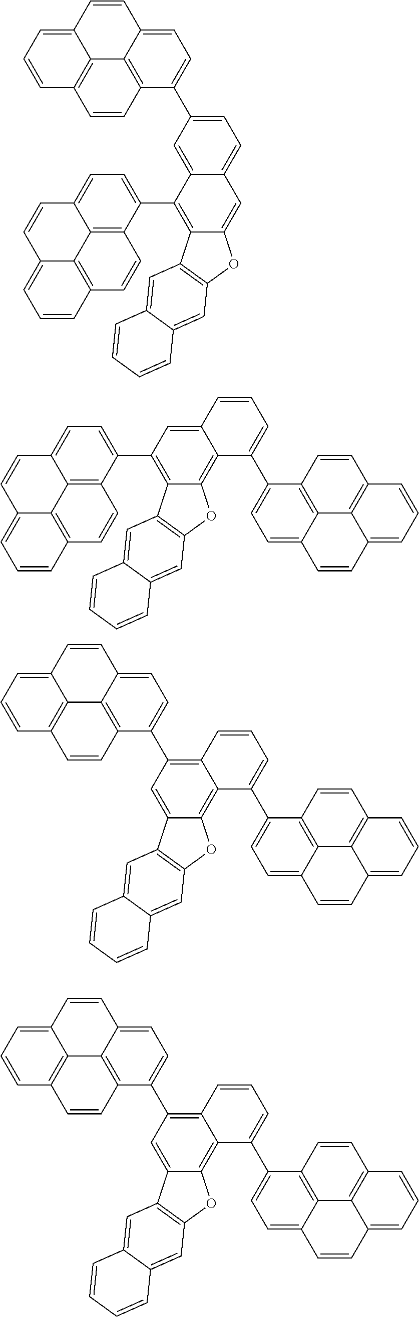

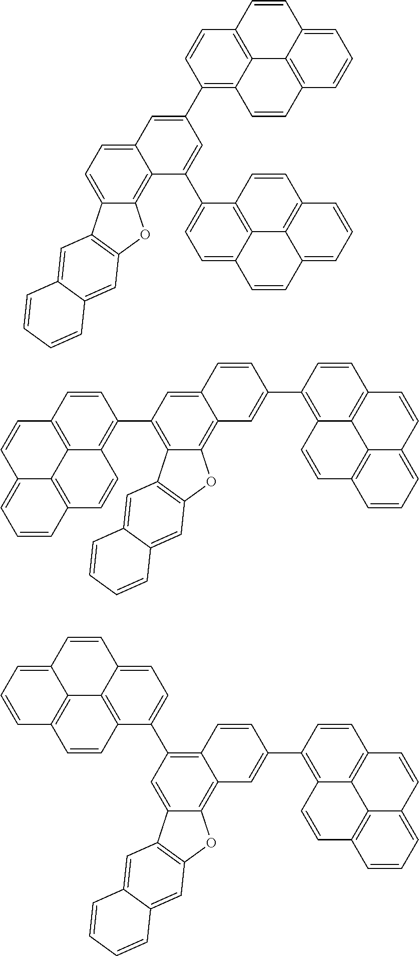

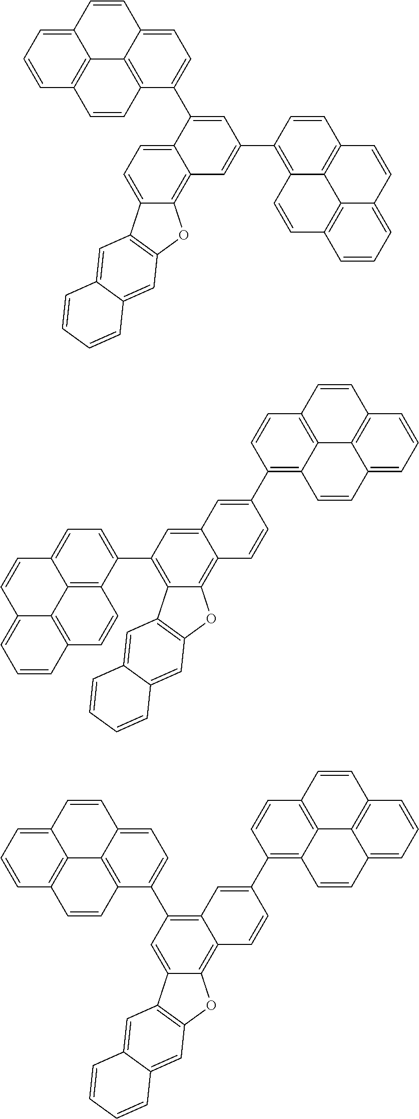 [SMILES: c1ccc2cc3c(cc2c1)oc1c2cc(-c4ccc5ccc6cccc7ccc4c5c67)cc(-c4ccc5ccc6cccc7ccc4c5c67)c2ccc31 ; c1ccc2cc3c(cc2c1)oc1c2ccc(-c4ccc5ccc6cccc7ccc4c5c67)cc2cc(-c2ccc4ccc5cccc6ccc2c4c56)c31 ; c1ccc2cc3c(cc2c1)oc1c2ccc(-c4ccc5ccc6cccc7ccc4c5c67)cc2c(-c2ccc4ccc5cccc6ccc2c4c56)cc31]

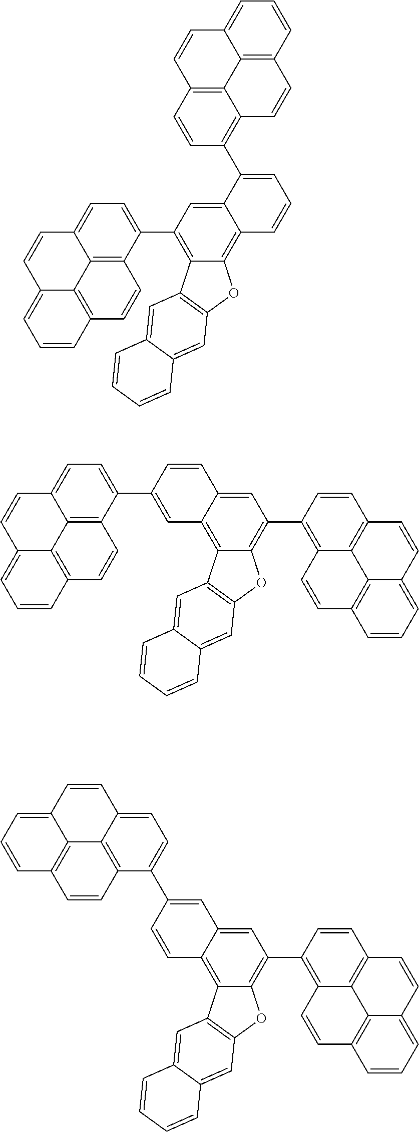 [SMILES: c1ccc2cc3c(cc2c1)oc1c2cccc(-c4ccc5ccc6cccc7ccc4c5c67)c2cc(-c2ccc4ccc5cccc6ccc2c4c56)c31 ; c1ccc2cc3c(cc2c1)oc1c(-c2ccc4ccc5cccc6ccc2c4c56)cc2ccc(-c4ccc5ccc6cccc7ccc4c5c67)cc2c13 ; c1ccc2cc3c(cc2c1)oc1c(-c2ccc4ccc5cccc6ccc2c4c56)cc2cc(-c4ccc5ccc6cccc7ccc4c5c67)ccc2c13]

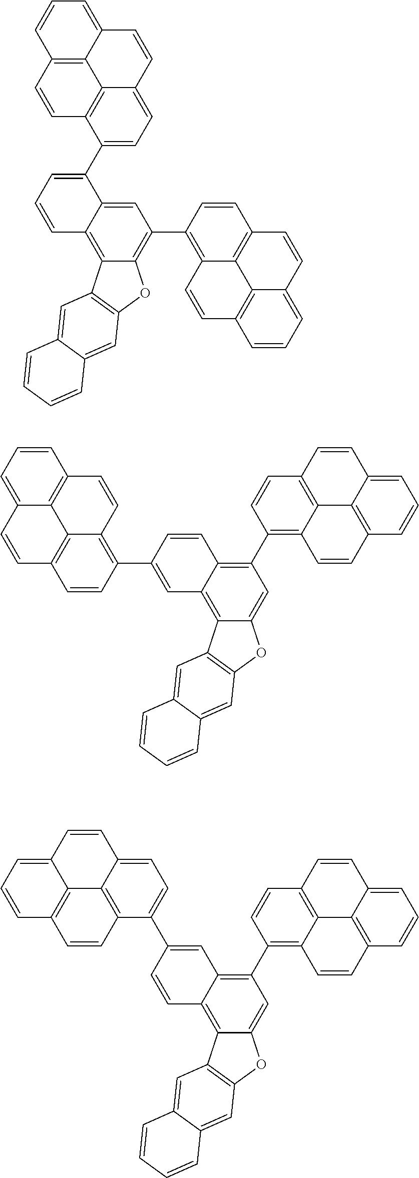

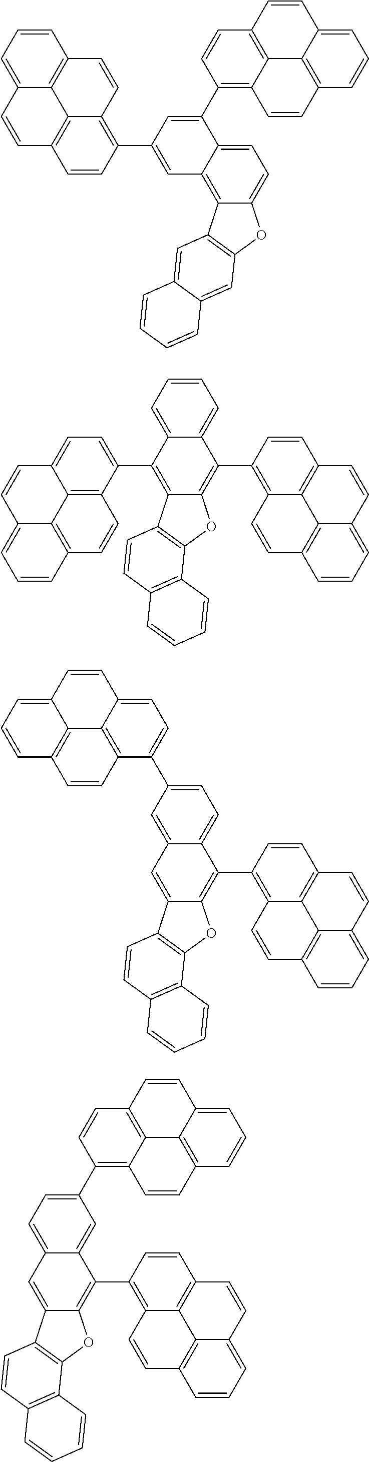

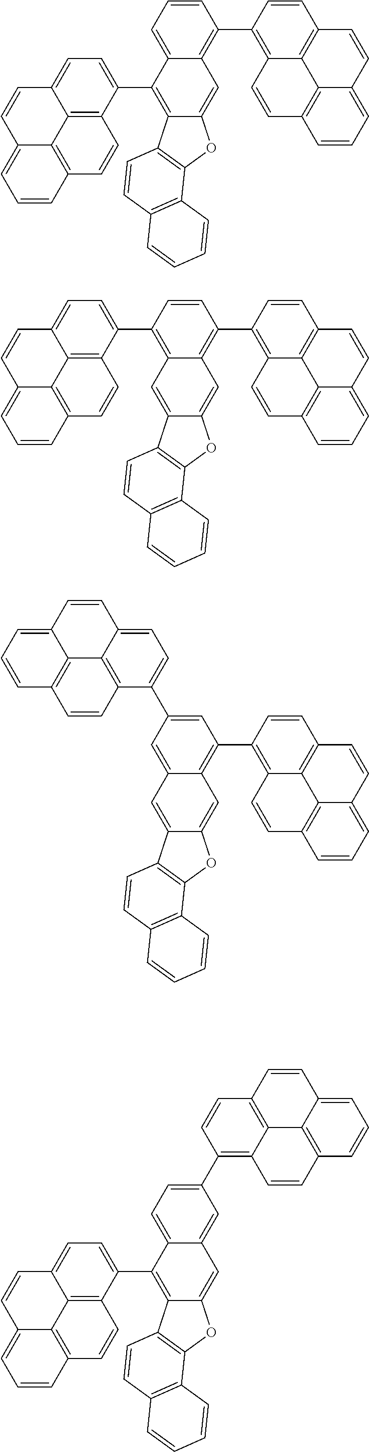

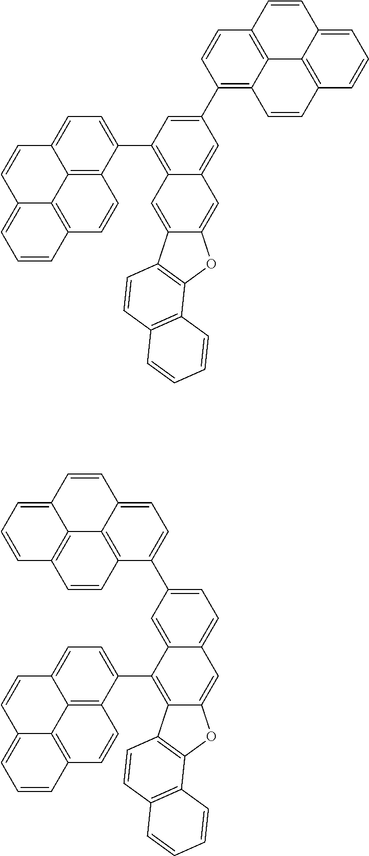 [SMILES: c1ccc2c(c1)ccc1c3cc4c(-c5ccc6ccc7cccc8ccc5c6c78)cc(-c5ccc6ccc7cccc8ccc5c6c78)cc4cc3oc21 ; c1ccc2c(c1)ccc1c2oc2cc3ccc(-c4ccc5ccc6cccc7ccc4c5c67)cc3c(-c3ccc4ccc5cccc6ccc3c4c56)c21]

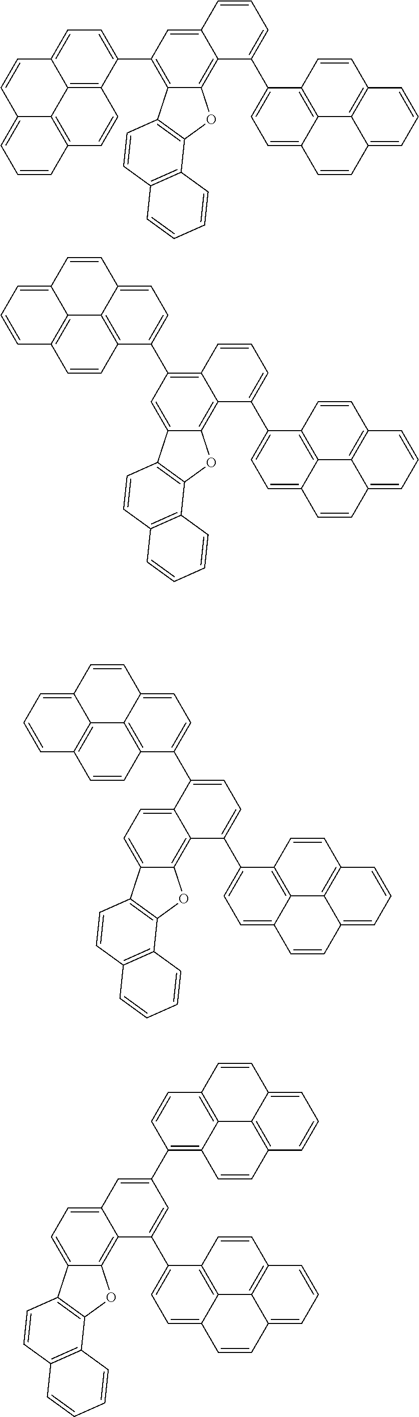 [SMILES: c1ccc2c(c1)ccc1c2oc2c3c(-c4ccc5ccc6cccc7ccc4c5c67)cccc3cc(-c3ccc4ccc5cccc6ccc3c4c56)c12 ; c1ccc2c(c1)ccc1c3cc(-c4ccc5ccc6cccc7ccc4c5c67)c4cccc(-c5ccc6ccc7cccc8ccc5c6c78)c4c3oc21 ; c1ccc2c(c1)ccc1c3ccc4c(-c5ccc6ccc7cccc8ccc5c6c78)ccc(-c5ccc6ccc7cccc8ccc5c6c78)c4c3oc21 ; c1ccc2c(c1)ccc1c3ccc4cc(-c5ccc6ccc7cccc8ccc5c6c78)cc(-c5ccc6ccc7cccc8ccc5c6c78)c4c3oc21]

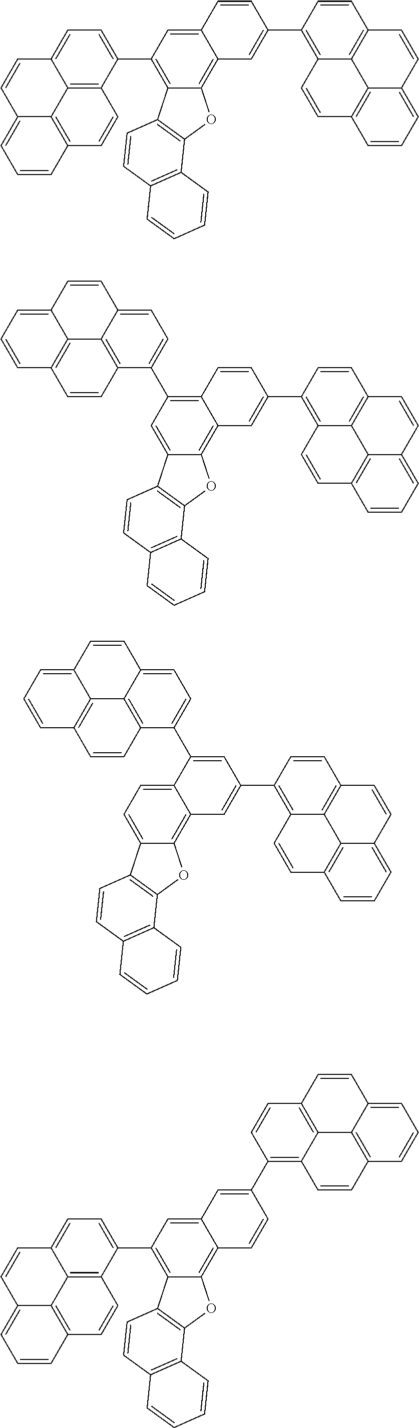 [SMILES: c1ccc2c(c1)ccc1c2oc2c3cc(-c4ccc5ccc6cccc7ccc4c5c67)ccc3cc(-c3ccc4ccc5cccc6ccc3c4c56)c12 ; c1ccc2c(c1)ccc1c3cc(-c4ccc5ccc6cccc7ccc4c5c67)c4ccc(-c5ccc6ccc7cccc8ccc5c6c78)cc4c3oc21 ; c1ccc2c(c1)ccc1c3ccc4c(-c5ccc6ccc7cccc8ccc5c6c78)cc(-c5ccc6ccc7cccc8ccc5c6c78)cc4c3oc21 ; c1ccc2c(c1)ccc1c2oc2c3ccc(-c4ccc5ccc6cccc7ccc4c5c67)cc3cc(-c3ccc4ccc5cccc6ccc3c4c56)c12]

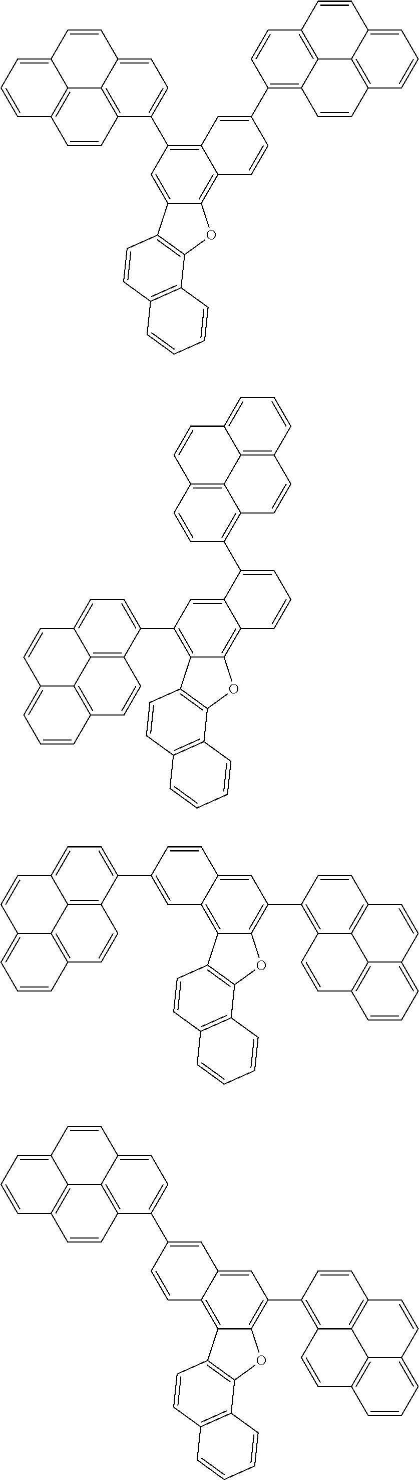

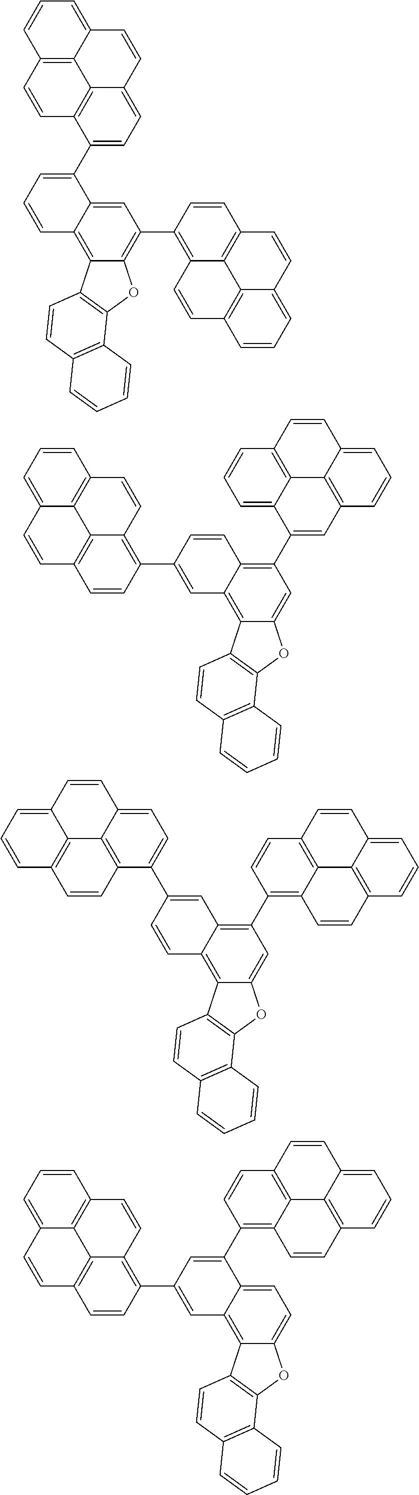

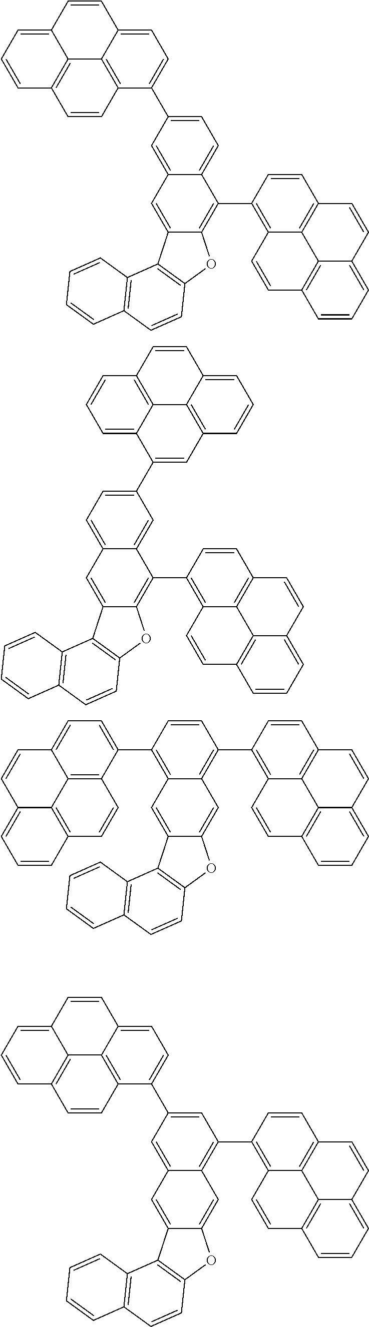

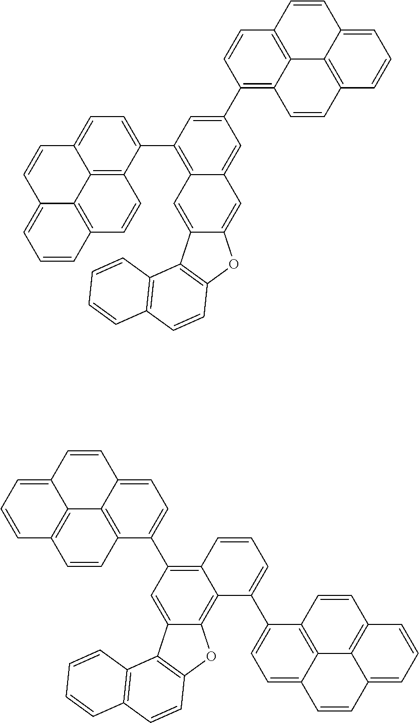 [SMILES: c1ccc2c(c1)ccc1oc3cc4cc(-c5ccc6ccc7cccc8ccc5c6c78)cc(-c5ccc6ccc7cccc8ccc5c6c78)c4cc3c12 ; c1ccc2c(c1)ccc1oc3c(cc(-c4ccc5ccc6cccc7ccc4c5c67)c4cccc(-c5ccc6ccc7cccc8ccc5c6c78)c43)c12]

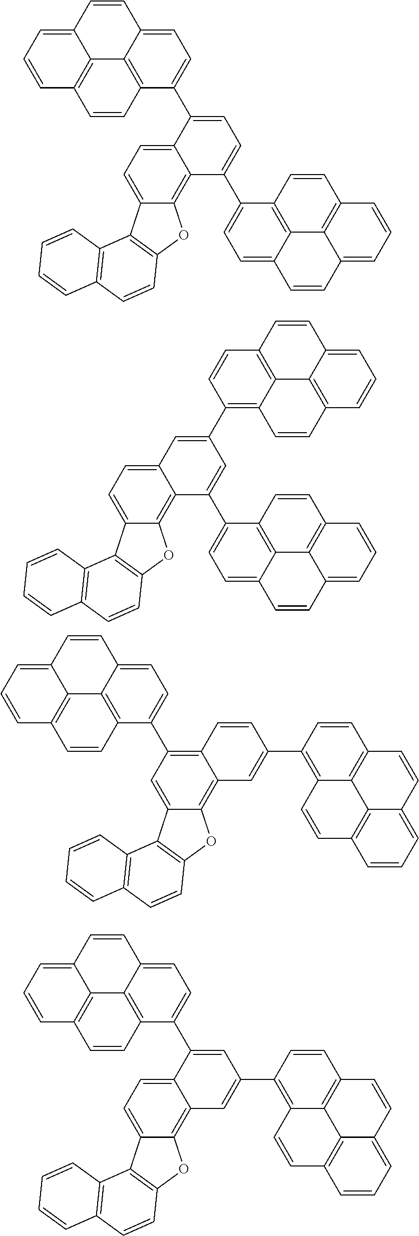

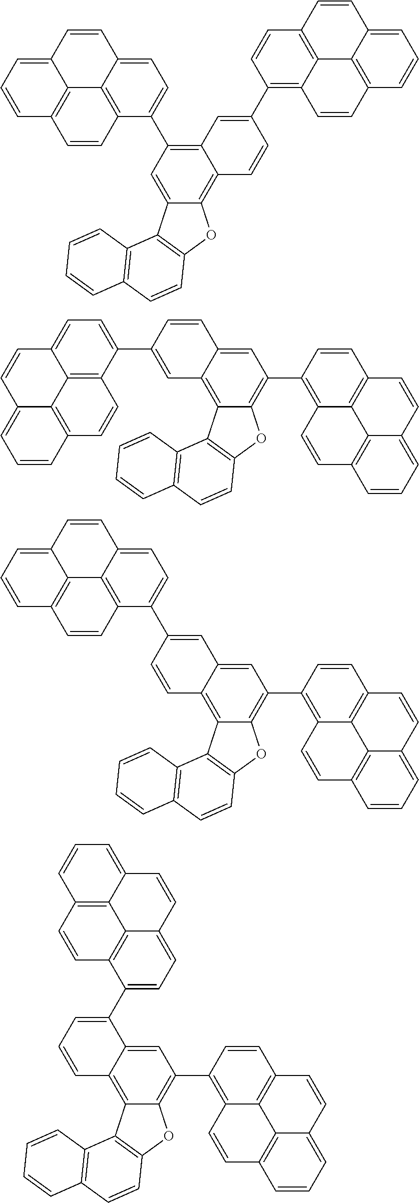

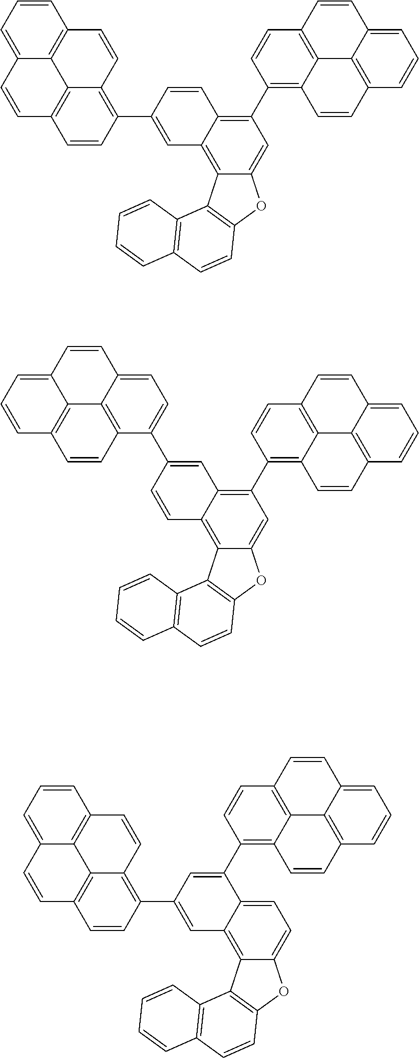 [SMILES: c1ccc2c(c1)ccc1oc3cc(-c4ccc5ccc6cccc7ccc4c5c67)c4ccc(-c5ccc6ccc7cccc8ccc5c6c78)cc4c3c12 ; c1ccc2c(c1)ccc1oc3cc(-c4ccc5ccc6cccc7ccc4c5c67)c4cc(-c5ccc6ccc7cccc8ccc5c6c78)ccc4c3c12 ; c1ccc2c(c1)ccc1oc3ccc4c(-c5ccc6ccc7cccc8ccc5c6c78)cc(-c5ccc6ccc7cccc8ccc5c6c78)cc4c3c12]

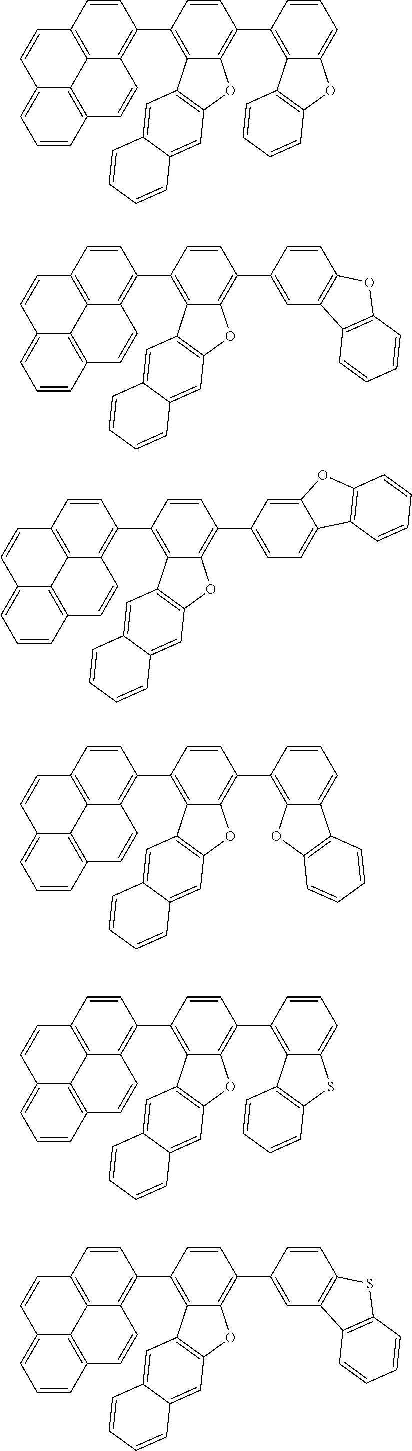

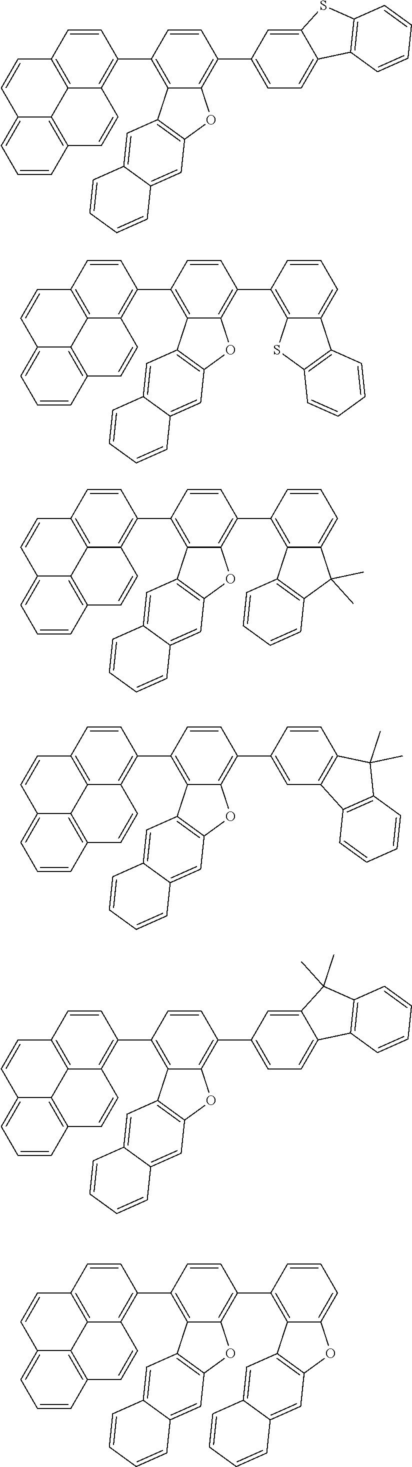

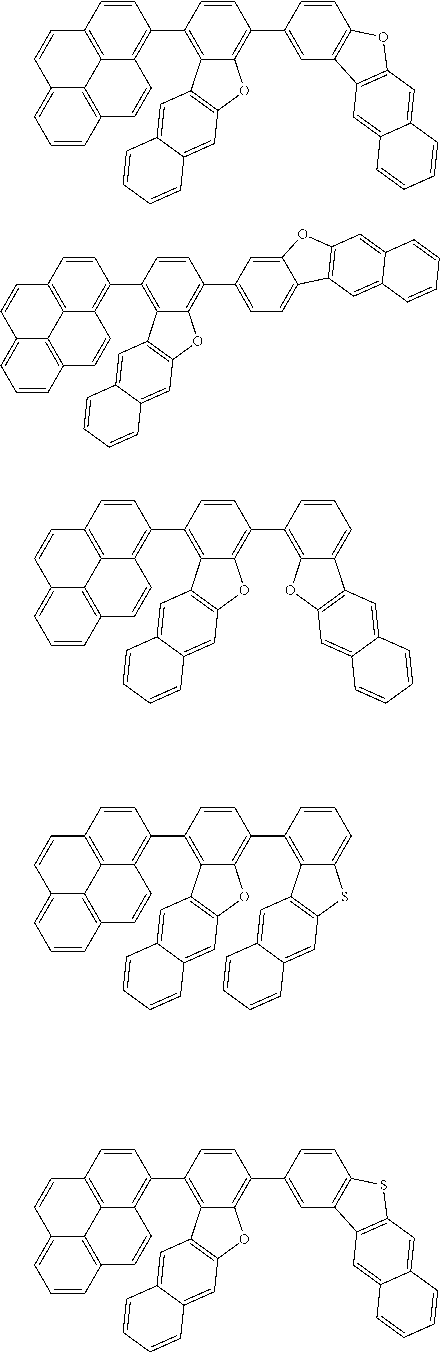 [SMILES: c1ccc2cc3c(cc2c1)oc1ccc(-c2ccc(-c4ccc5ccc6cccc7ccc4c5c67)c4c2oc2cc5ccccc5cc24)cc13 ; c1ccc2cc3c(cc2c1)oc1cc(-c2ccc(-c4ccc5ccc6cccc7ccc4c5c67)c4c2oc2cc5ccccc5cc24)ccc13 ; c1ccc2cc3c(cc2c1)oc1c(-c2ccc(-c4ccc5ccc6cccc7ccc4c5c67)c4c2oc2cc5ccccc5cc24)cccc13 ; c1ccc2cc3c(cc2c1)oc1c(-c2cccc4sc5cc6ccccc6cc5c24)ccc(-c2ccc4ccc5cccc6ccc2c4c56)c13 ; c1ccc2cc3c(cc2c1)oc1c(-c2ccc4sc5cc6ccccc6cc5c4c2)ccc(-c2ccc4ccc5cccc6ccc2c4c56)c13]

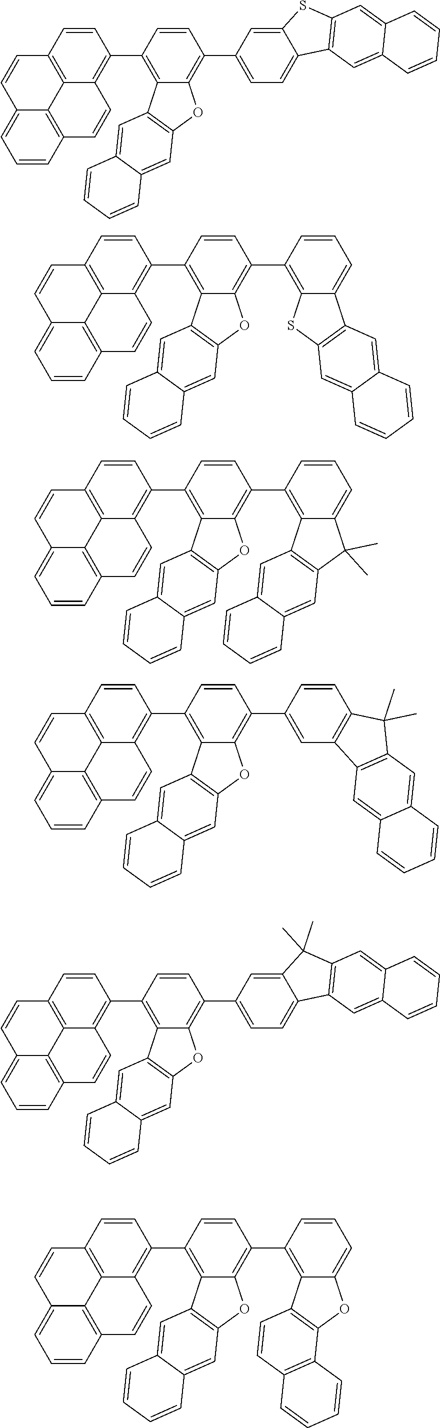

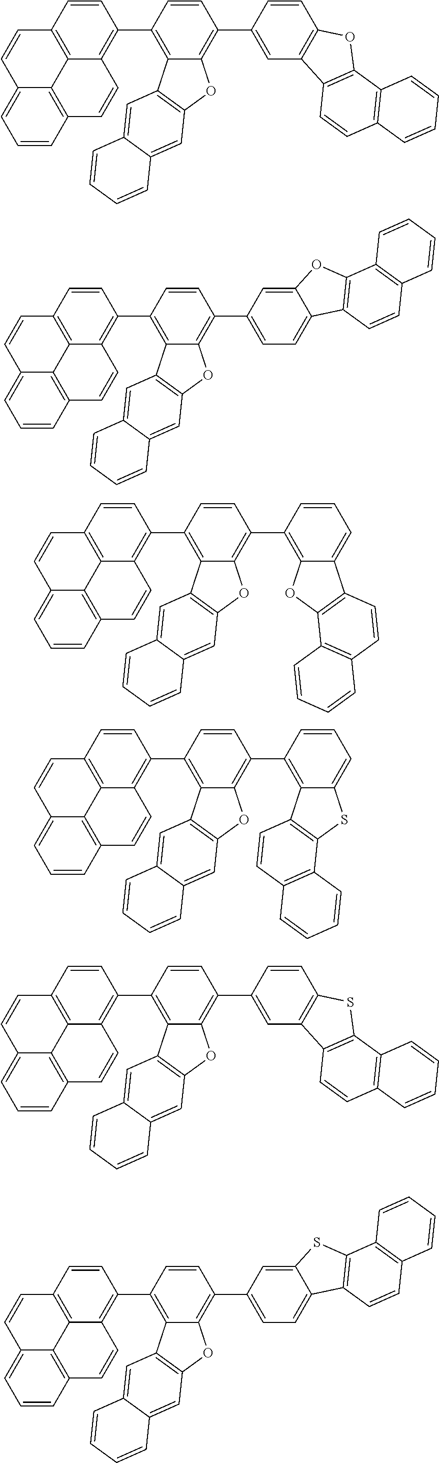

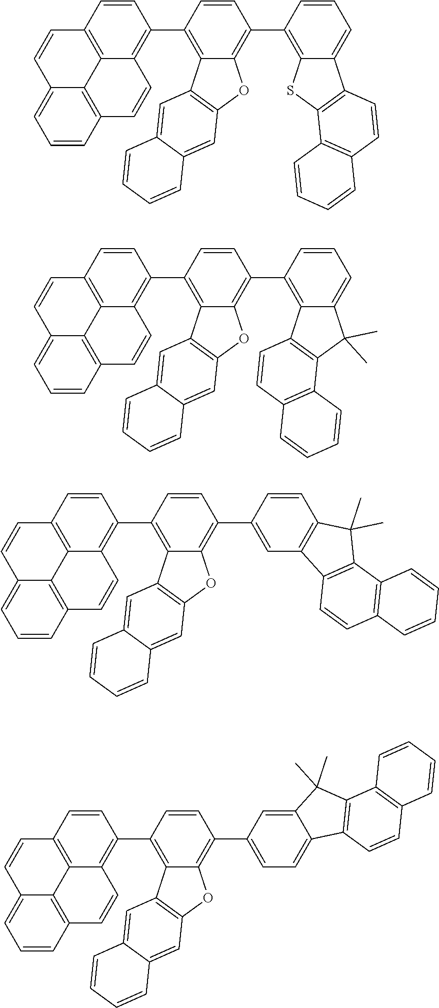

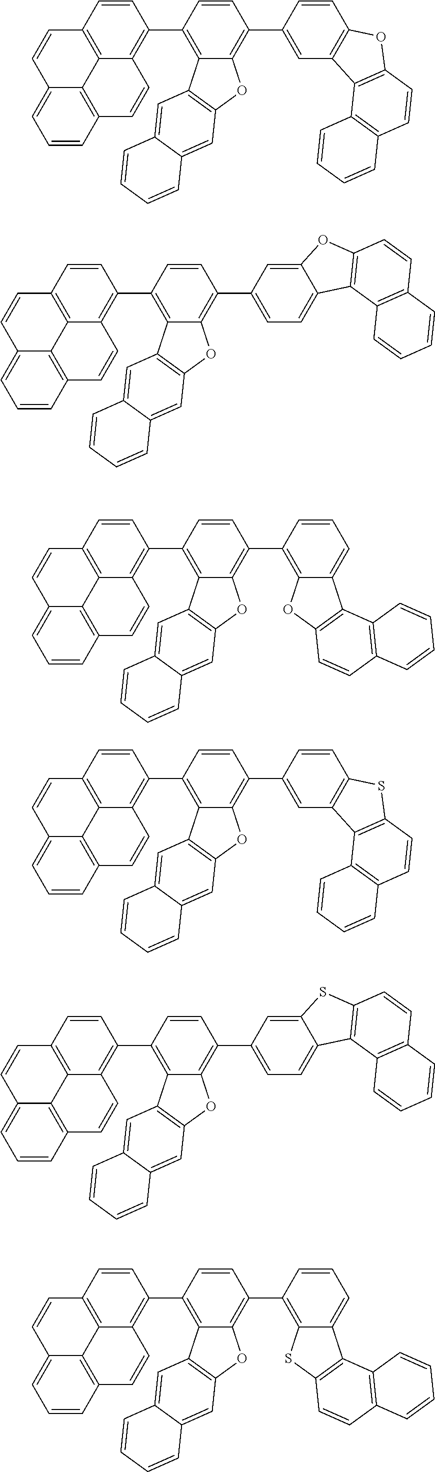

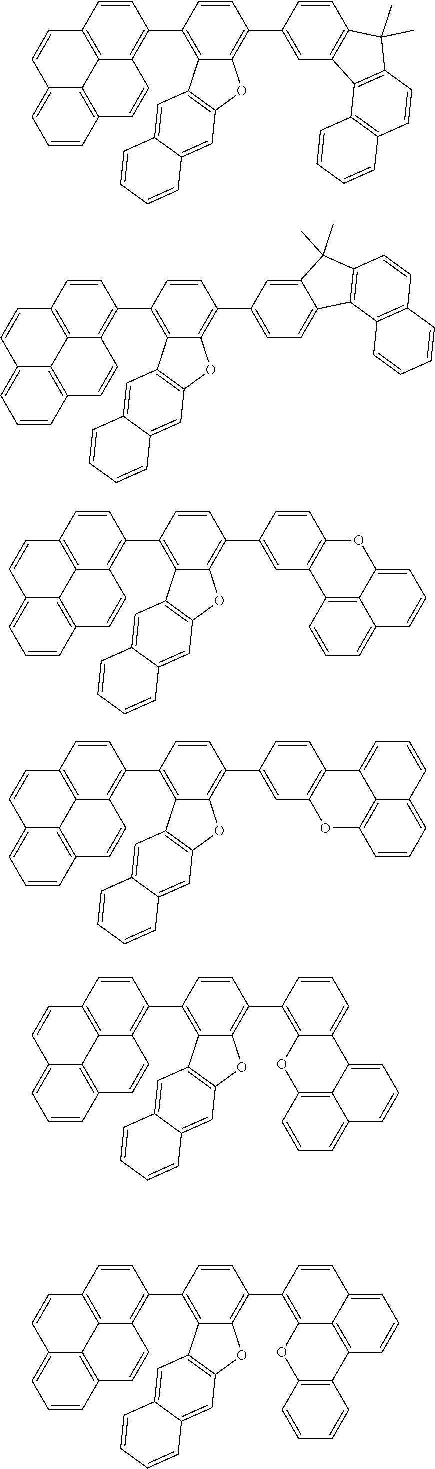

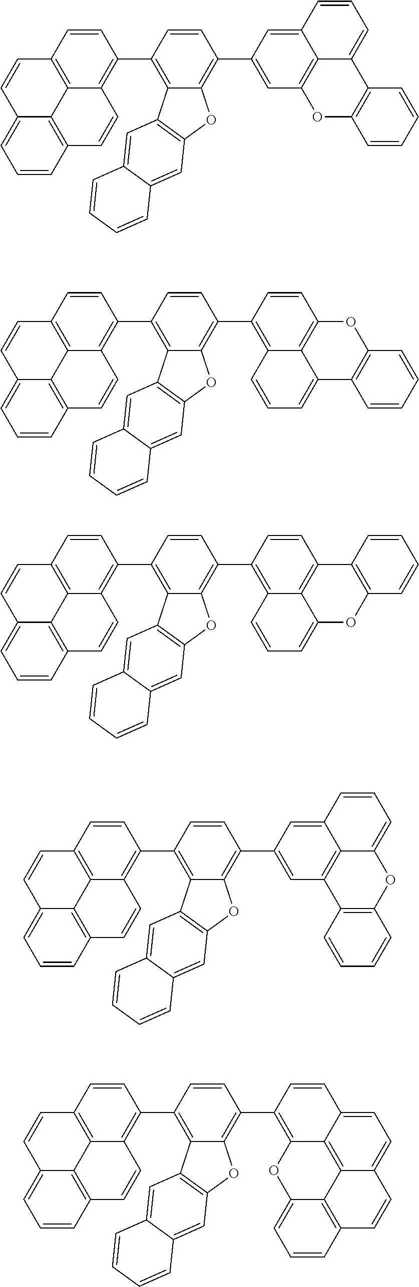

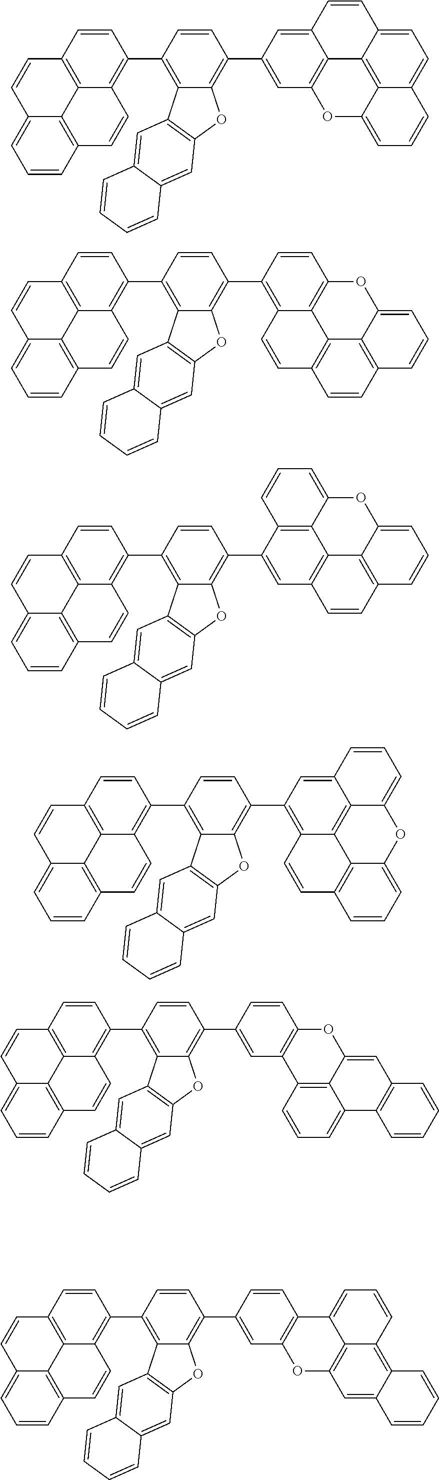

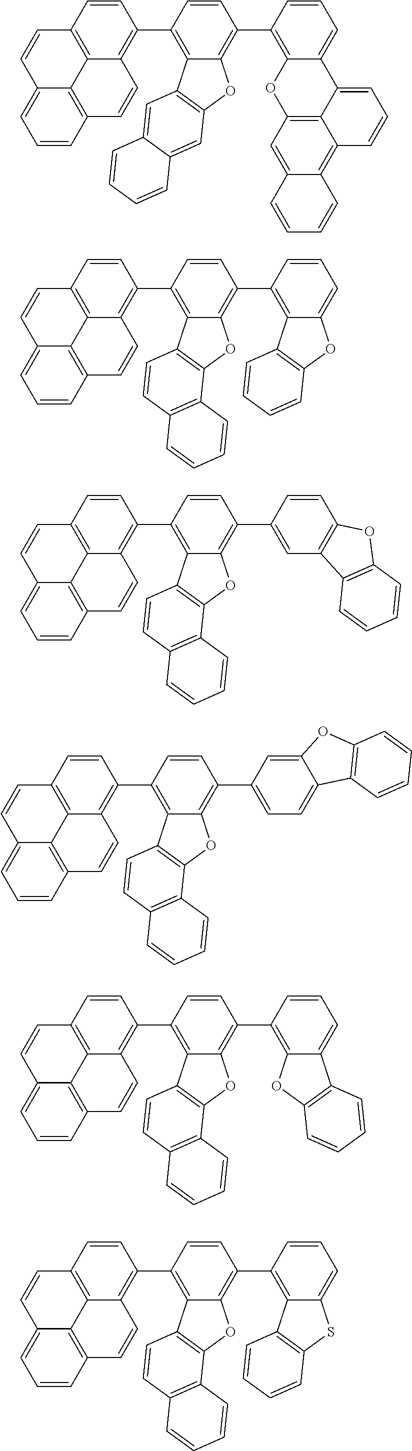

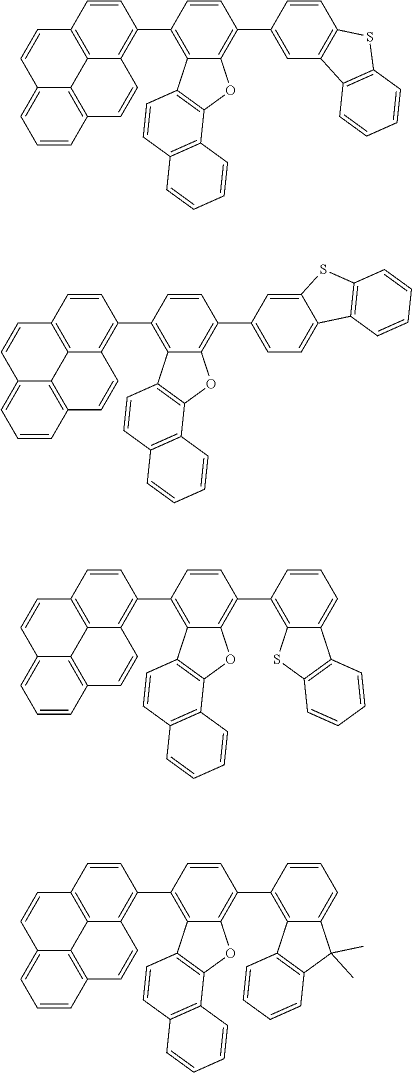 [SMILES: c1ccc2c(c1)ccc1c2oc2c(-c3ccc4sc5ccccc5c4c3)ccc(-c3ccc4ccc5cccc6ccc3c4c56)c21 ; c1ccc2c(c1)ccc1c2oc2c(-c3ccc4c(c3)sc3ccccc34)ccc(-c3ccc4ccc5cccc6ccc3c4c56)c21 ; c1ccc2c(c1)ccc1c2oc2c(-c3cccc4c3sc3ccccc34)ccc(-c3ccc4ccc5cccc6ccc3c4c56)c21 ; CC1(C)c2ccccc2-c2c(-c3ccc(-c4ccc5ccc6cccc7ccc4c5c67)c4c3oc3c5ccccc5ccc34)cccc21]

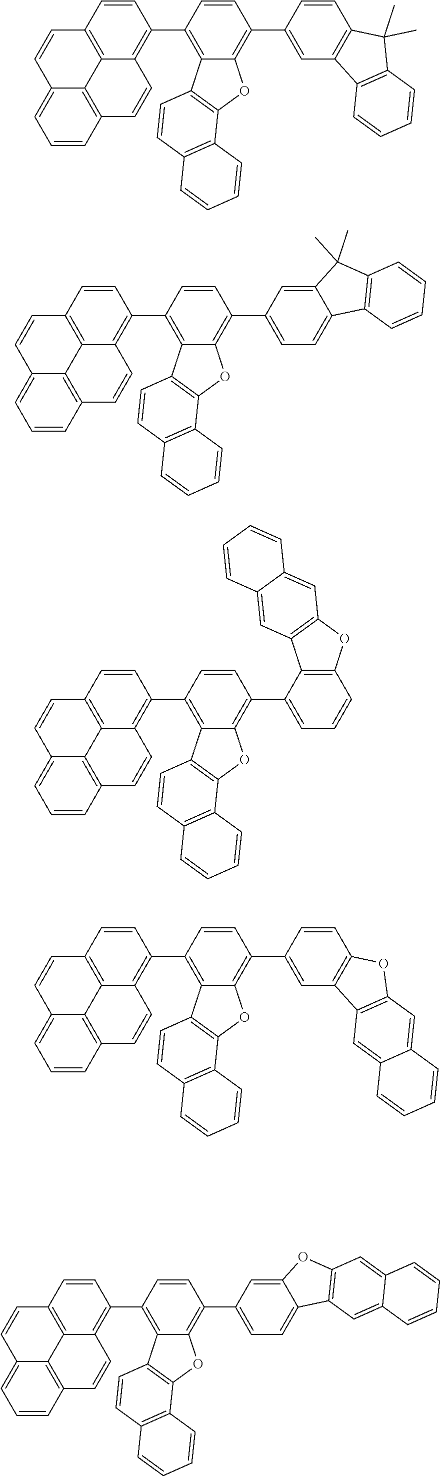 [SMILES: CC1(C)c2ccccc2-c2cc(-c3ccc(-c4ccc5ccc6cccc7ccc4c5c67)c4c3oc3c5ccccc5ccc34)ccc21 ; CC1(C)c2ccccc2-c2ccc(-c3ccc(-c4ccc5ccc6cccc7ccc4c5c67)c4c3oc3c5ccccc5ccc34)cc21 ; c1ccc2cc3c(cc2c1)oc1cccc(-c2ccc(-c4ccc5ccc6cccc7ccc4c5c67)c4c2oc2c5ccccc5ccc24)c13 ; c1ccc2cc3c(cc2c1)oc1ccc(-c2ccc(-c4ccc5ccc6cccc7ccc4c5c67)c4c2oc2c5ccccc5ccc24)cc13 ; c1ccc2cc3c(cc2c1)oc1cc(-c2ccc(-c4ccc5ccc6cccc7ccc4c5c67)c4c2oc2c5ccccc5ccc24)ccc13]

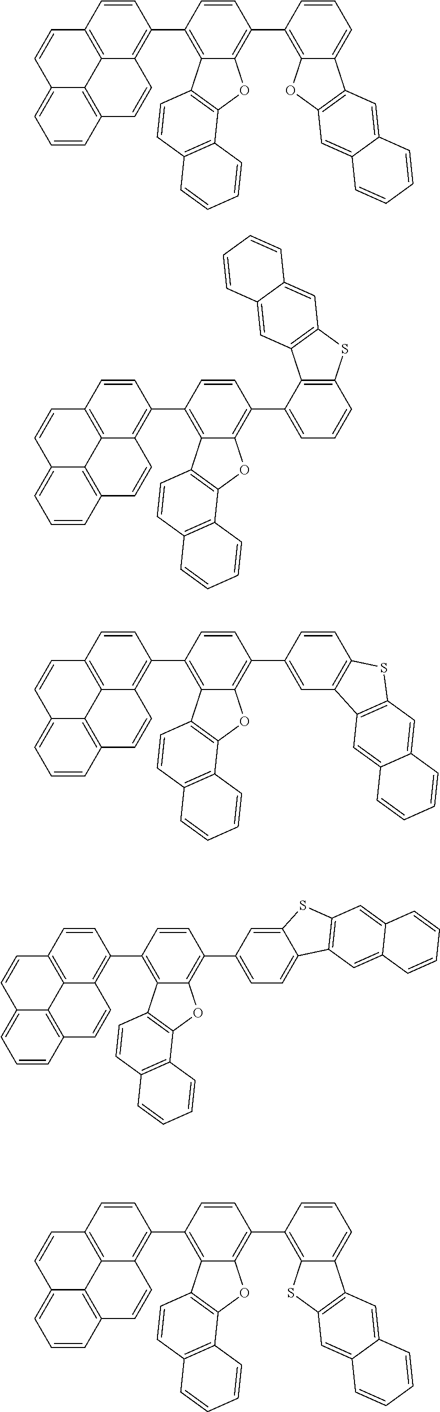

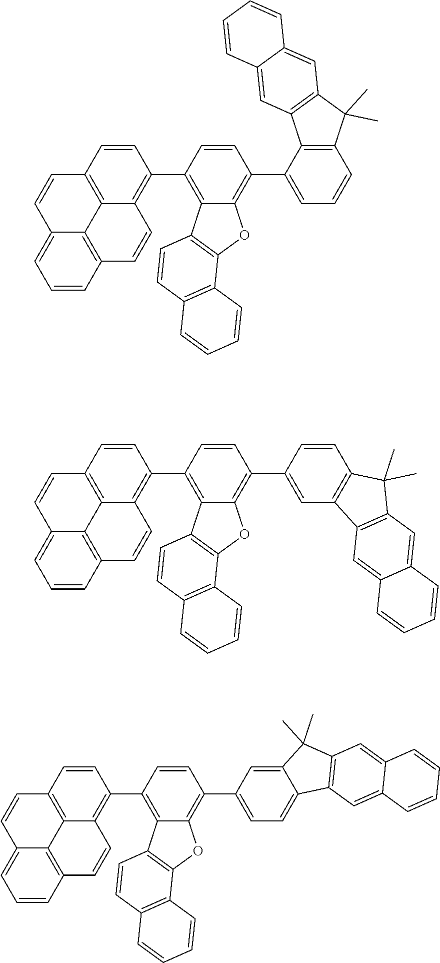 [SMILES: CC1(C)c2cc3ccccc3cc2-c2c(-c3ccc(-c4ccc5ccc6cccc7ccc4c5c67)c4c3oc3c5ccccc5ccc34)cccc21 ; CC1(C)c2ccc(-c3ccc(-c4ccc5ccc6cccc7ccc4c5c67)c4c3oc3c5ccccc5ccc34)cc2-c2cc3ccccc3cc21 ; CC1(C)c2cc(-c3ccc(-c4ccc5ccc6cccc7ccc4c5c67)c4c3oc3c5ccccc5ccc34)ccc2-c2cc3ccccc3cc21]

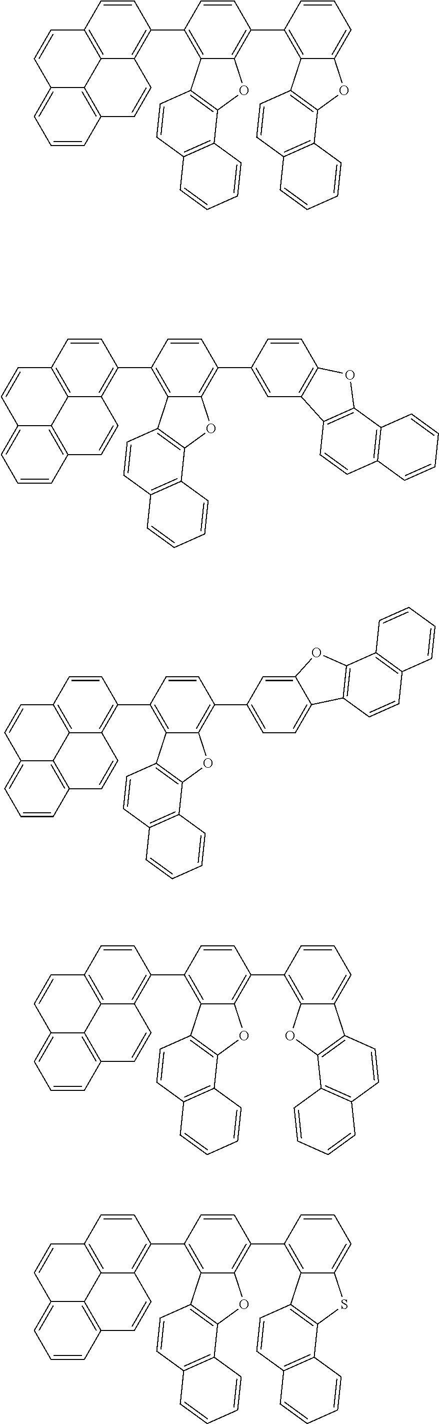 [SMILES: c1ccc2c(c1)ccc1c2oc2cccc(-c3ccc(-c4ccc5ccc6cccc7ccc4c5c67)c4c3oc3c5ccccc5ccc34)c21 ; c1ccc2c(c1)ccc1c3cc(-c4ccc(-c5ccc6ccc7cccc8ccc5c6c78)c5c4oc4c6ccccc6ccc45)ccc3oc21 ; c1ccc2c(c1)ccc1c3ccc(-c4ccc(-c5ccc6ccc7cccc8ccc5c6c78)c5c4oc4c6ccccc6ccc45)cc3oc21 ; c1ccc2c(c1)ccc1c3cccc(-c4ccc(-c5ccc6ccc7cccc8ccc5c6c78)c5c4oc4c6ccccc6ccc45)c3oc21 ; c1ccc2c(c1)ccc1c2oc2c(-c3cccc4sc5c6ccccc6ccc5c34)ccc(-c3ccc4ccc5cccc6ccc3c4c56)c21]

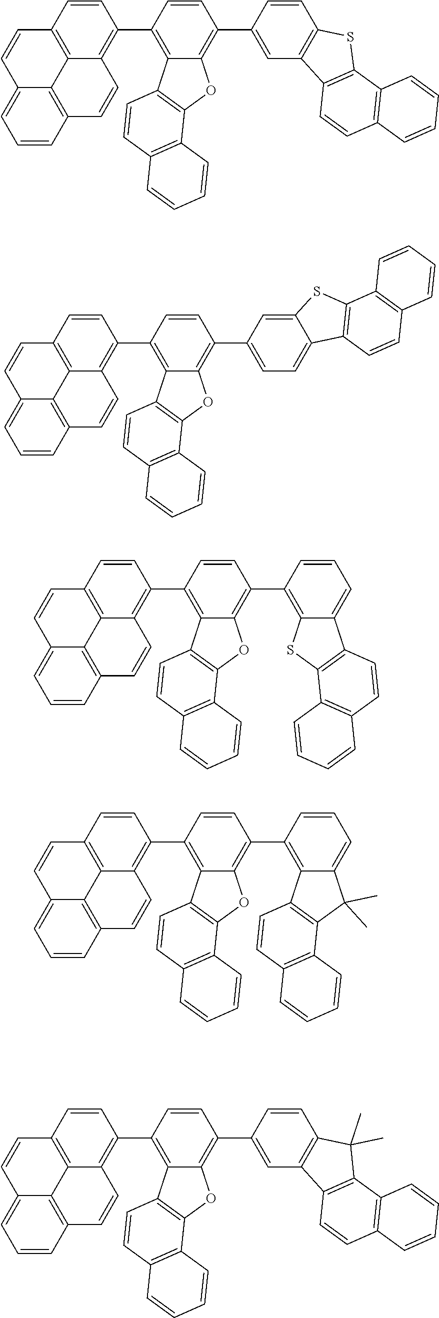

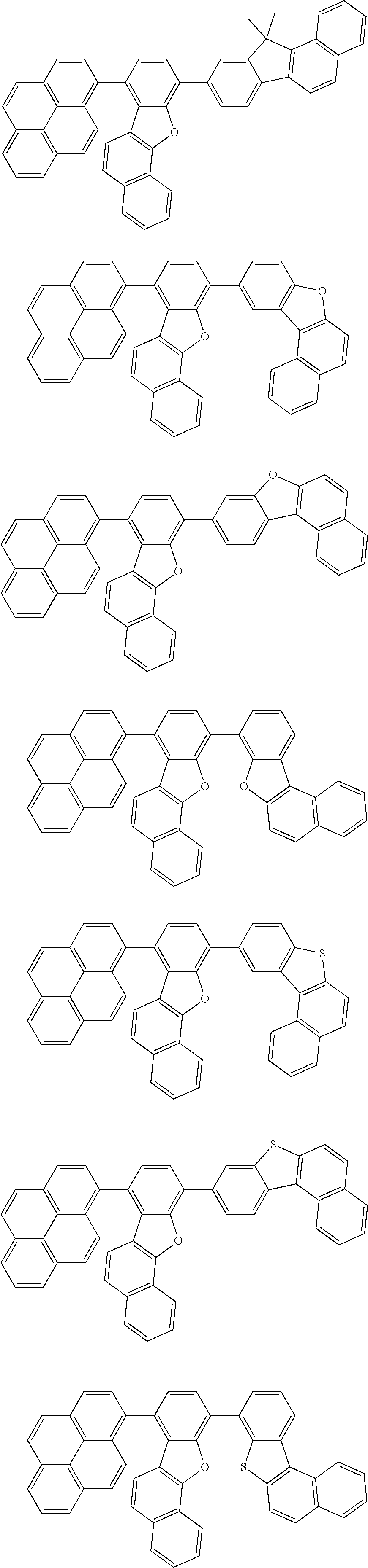

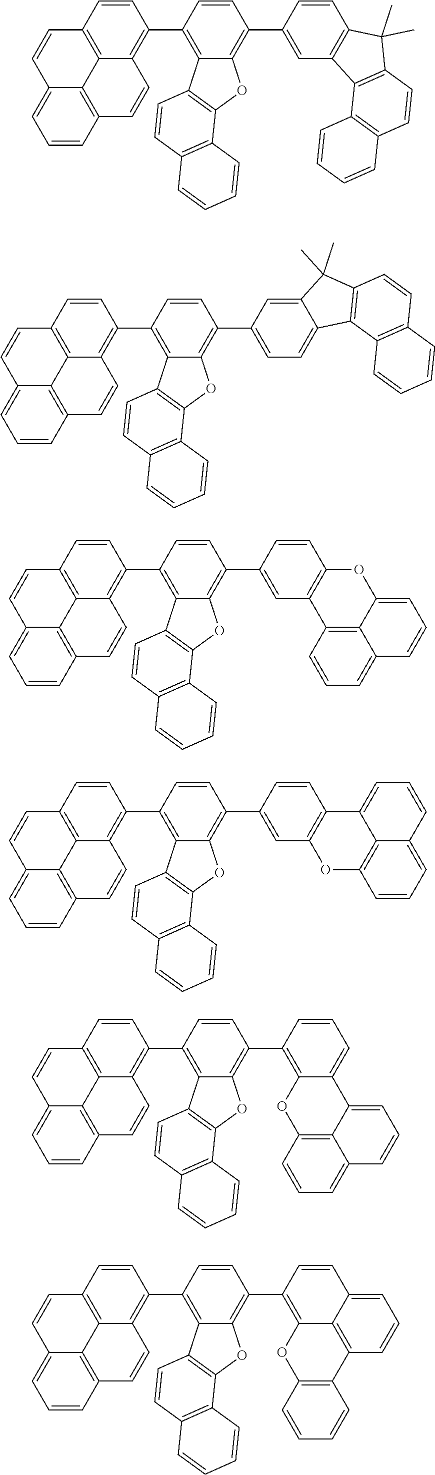

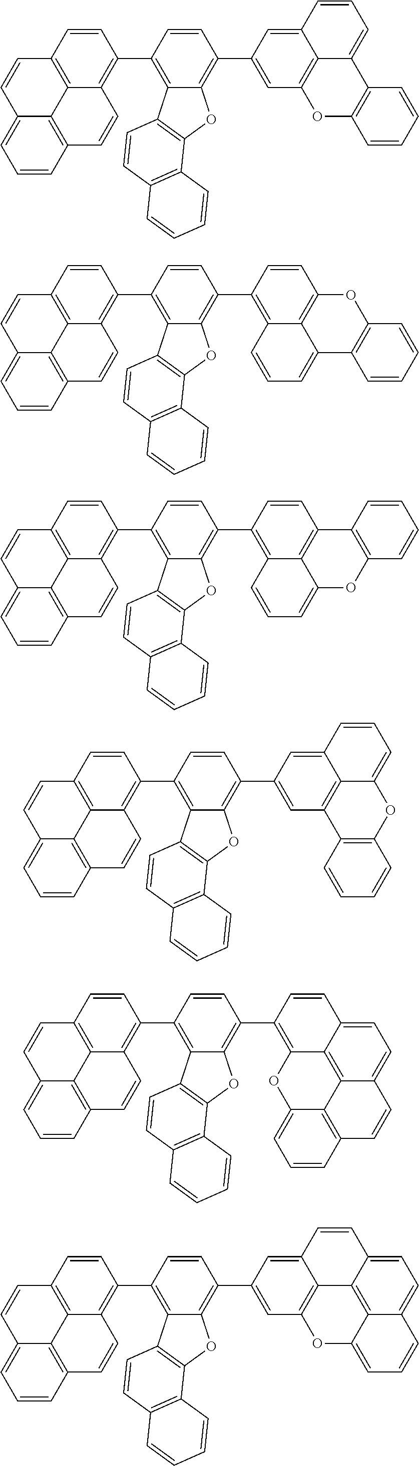

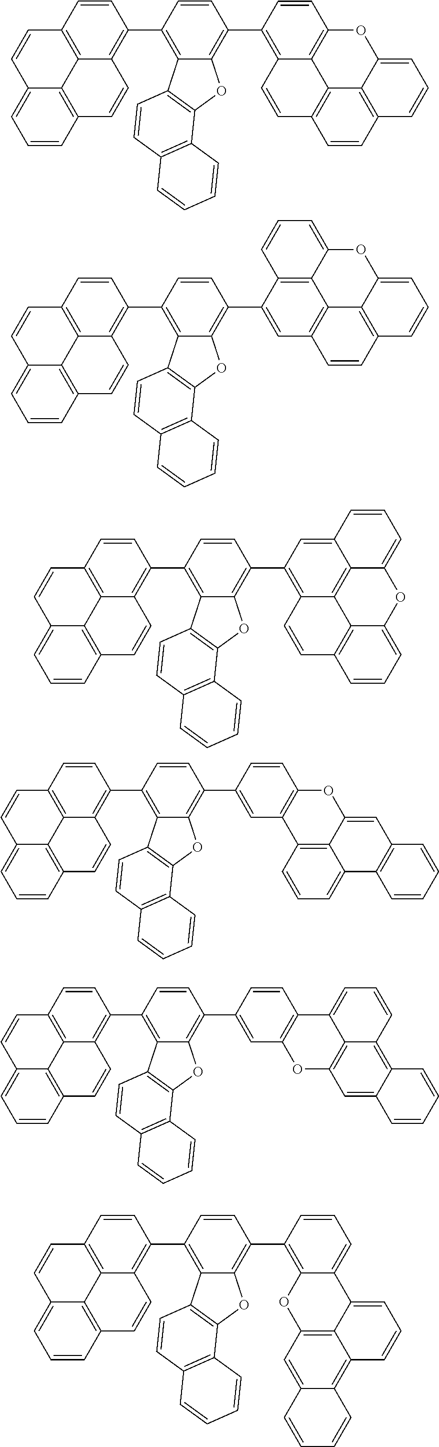

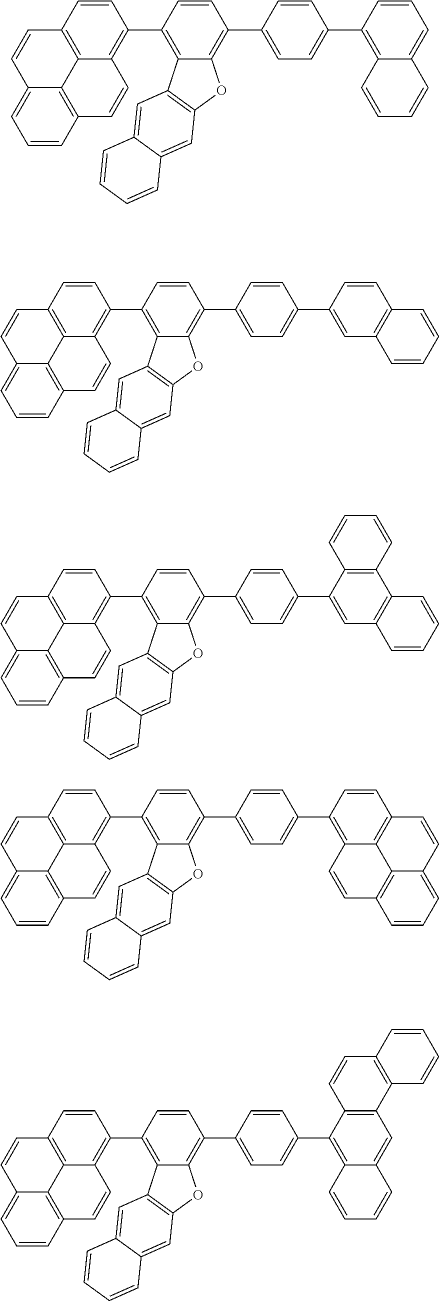

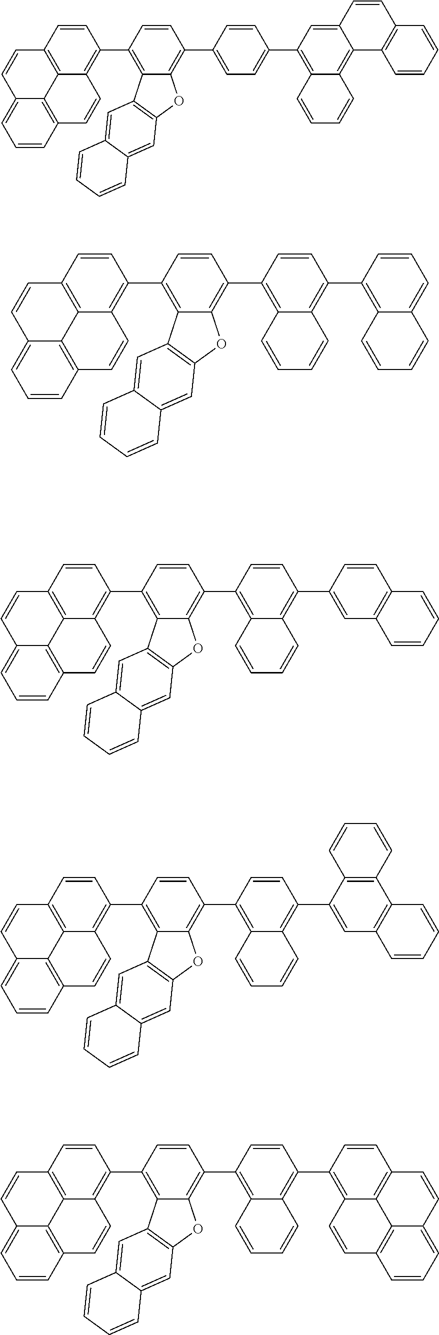 [SMILES: c1ccc2cc3c(cc2c1)oc1c(-c2ccc(-c4cc5ccc6ccccc6c5c5ccccc45)cc2)ccc(-c2ccc4ccc5cccc6ccc2c4c56)c13 ; c1ccc2cc3c(cc2c1)oc1c(-c2ccc(-c4cccc5ccccc45)c4ccccc24)ccc(-c2ccc4ccc5cccc6ccc2c4c56)c13 ; c1ccc2cc(-c3ccc(-c4ccc(-c5ccc6ccc7cccc8ccc5c6c78)c5c4oc4cc6ccccc6cc45)c4ccccc34)ccc2c1 ; c1ccc2cc3c(cc2c1)oc1c(-c2ccc(-c4cc5ccccc5c5ccccc45)c4ccccc24)ccc(-c2ccc4ccc5cccc6ccc2c4c56)c13 ; c1ccc2cc3c(cc2c1)oc1c(-c2ccc(-c4ccc5ccc6cccc7ccc4c5c67)c4ccccc24)ccc(-c2ccc4ccc5cccc6ccc2c4c56)c13]

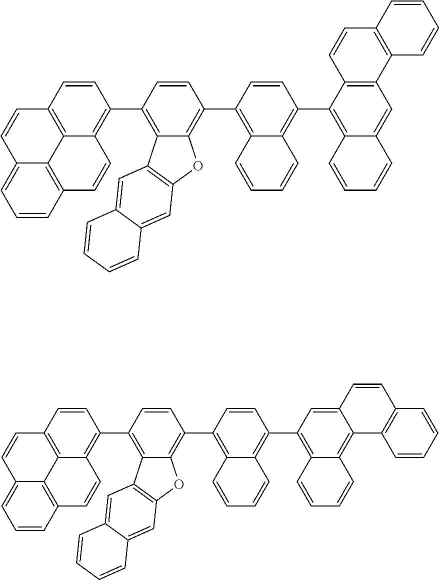 [SMILES: c1ccc2cc3c(cc2c1)oc1c(-c2ccc(-c4c5ccccc5cc5c4ccc4ccccc45)c4ccccc24)ccc(-c2ccc4ccc5cccc6ccc2c4c56)c13 ; c1ccc2cc3c(cc2c1)oc1c(-c2ccc(-c4cc5ccc6ccccc6c5c5ccccc45)c4ccccc24)ccc(-c2ccc4ccc5cccc6ccc2c4c56)c13]

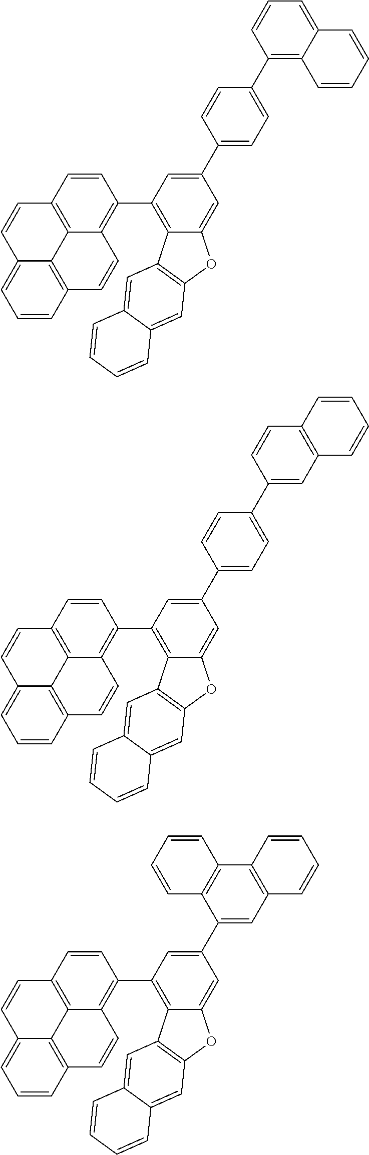 [SMILES: c1ccc2cc3c(cc2c1)oc1cc(-c2ccc(-c4cccc5ccccc45)cc2)cc(-c2ccc4ccc5cccc6ccc2c4c56)c13 ; c1ccc2cc(-c3ccc(-c4cc(-c5ccc6ccc7cccc8ccc5c6c78)c5c(c4)oc4cc6ccccc6cc45)cc3)ccc2c1 ; c1ccc2cc3c(cc2c1)oc1cc(-c2cc4ccccc4c4ccccc24)cc(-c2ccc4ccc5cccc6ccc2c4c56)c13]

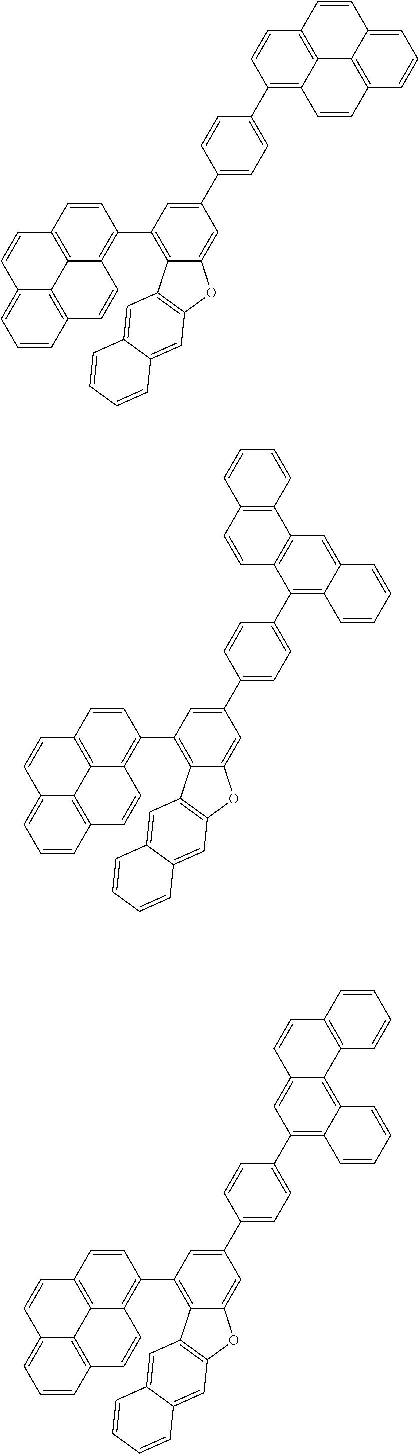 [SMILES: c1ccc2cc3c(cc2c1)oc1cc(-c2ccc(-c4ccc5ccc6cccc7ccc4c5c67)cc2)cc(-c2ccc4ccc5cccc6ccc2c4c56)c13 ; c1ccc2cc3c(cc2c1)oc1cc(-c2ccc(-c4c5ccccc5cc5c4ccc4ccccc45)cc2)cc(-c2ccc4ccc5cccc6ccc2c4c56)c13 ; c1ccc2cc3c(cc2c1)oc1cc(-c2ccc(-c4cc5ccc6ccccc6c5c5ccccc45)cc2)cc(-c2ccc4ccc5cccc6ccc2c4c56)c13]

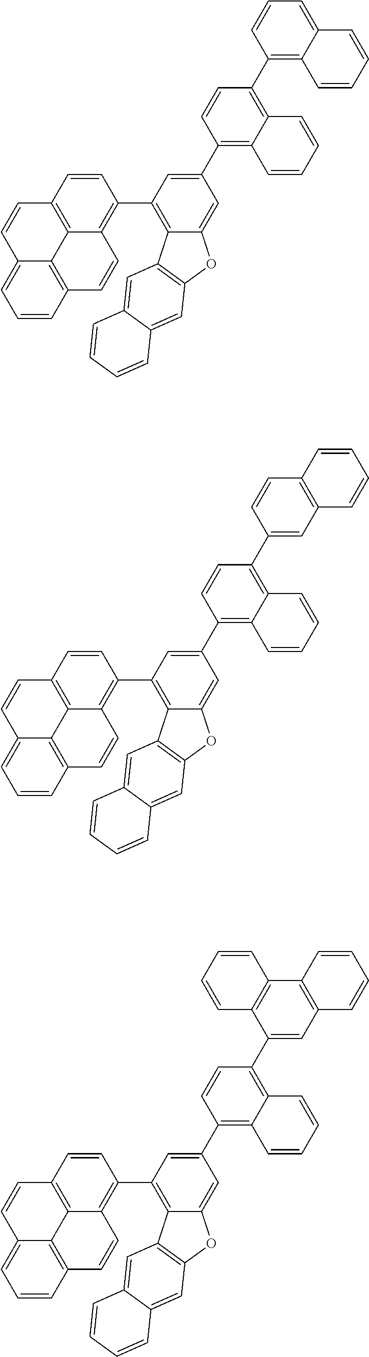 [SMILES: c1ccc2cc3c(cc2c1)oc1cc(-c2ccc(-c4cccc5ccccc45)c4ccccc24)cc(-c2ccc4ccc5cccc6ccc2c4c56)c13 ; c1ccc2cc(-c3ccc(-c4cc(-c5ccc6ccc7cccc8ccc5c6c78)c5c(c4)oc4cc6ccccc6cc45)c4ccccc34)ccc2c1 ; c1ccc2cc3c(cc2c1)oc1cc(-c2ccc(-c4cc5ccccc5c5ccccc45)c4ccccc24)cc(-c2ccc4ccc5cccc6ccc2c4c56)c13]

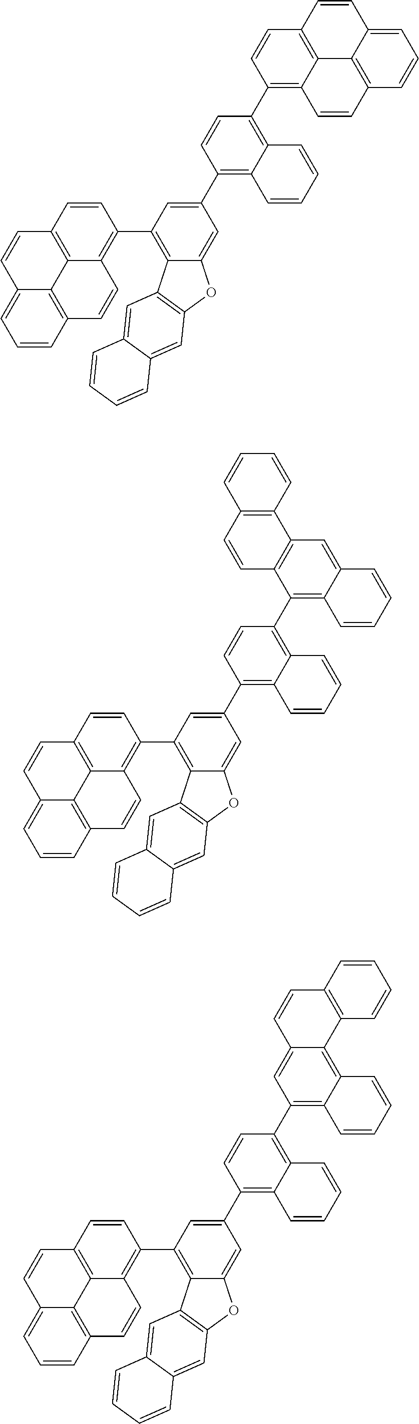 [SMILES: c1ccc2cc3c(cc2c1)oc1cc(-c2ccc(-c4ccc5ccc6cccc7ccc4c5c67)c4ccccc24)cc(-c2ccc4ccc5cccc6ccc2c4c56)c13 ; c1ccc2cc3c(cc2c1)oc1cc(-c2ccc(-c4c5ccccc5cc5c4ccc4ccccc45)c4ccccc24)cc(-c2ccc4ccc5cccc6ccc2c4c56)c13 ; c1ccc2cc3c(cc2c1)oc1cc(-c2ccc(-c4cc5ccc6ccccc6c5c5ccccc45)c4ccccc24)cc(-c2ccc4ccc5cccc6ccc2c4c56)c13]

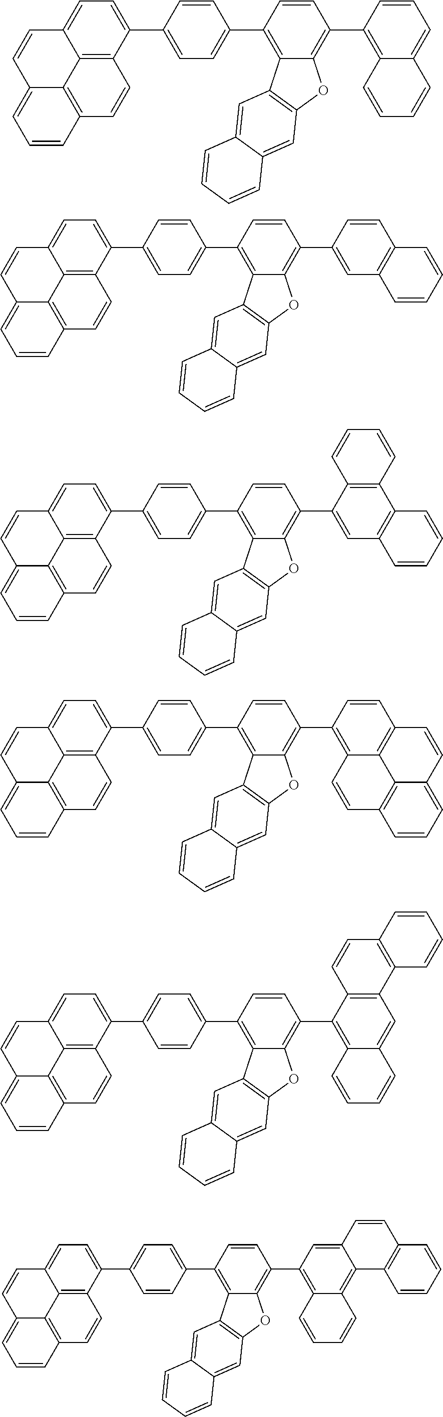

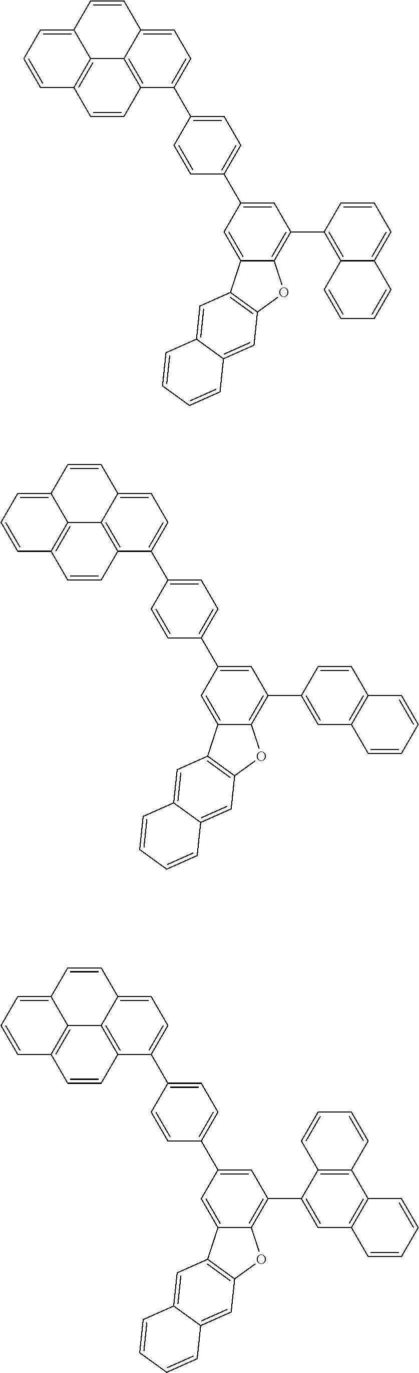 [SMILES: c1ccc2cc3c(cc2c1)oc1c(-c2cccc4ccccc24)cc(-c2ccc(-c4ccc5ccc6cccc7ccc4c5c67)cc2)cc13 ; c1ccc2cc(-c3cc(-c4ccc(-c5ccc6ccc7cccc8ccc5c6c78)cc4)cc4c3oc3cc5ccccc5cc34)ccc2c1 ; c1ccc2cc3c(cc2c1)oc1c(-c2cc4ccccc4c4ccccc24)cc(-c2ccc(-c4ccc5ccc6cccc7ccc4c5c67)cc2)cc13]

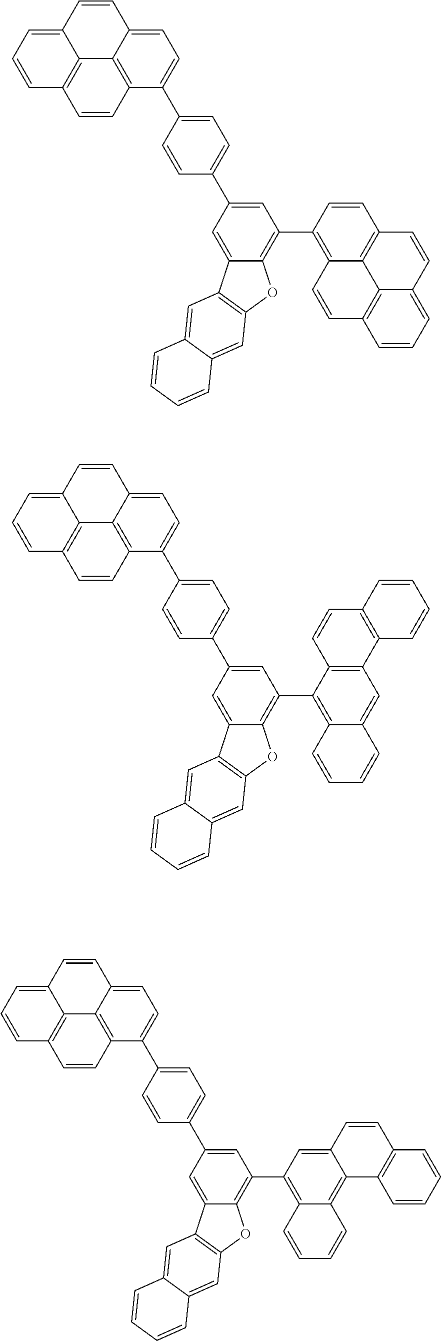 [SMILES: c1ccc2cc3c(cc2c1)oc1c(-c2ccc4ccc5cccc6ccc2c4c56)cc(-c2ccc(-c4ccc5ccc6cccc7ccc4c5c67)cc2)cc13 ; c1ccc2cc3c(cc2c1)oc1c(-c2c4ccccc4cc4c2ccc2ccccc24)cc(-c2ccc(-c4ccc5ccc6cccc7ccc4c5c67)cc2)cc13 ; c1ccc2cc3c(cc2c1)oc1c(-c2cc4ccc5ccccc5c4c4ccccc24)cc(-c2ccc(-c4ccc5ccc6cccc7ccc4c5c67)cc2)cc13]

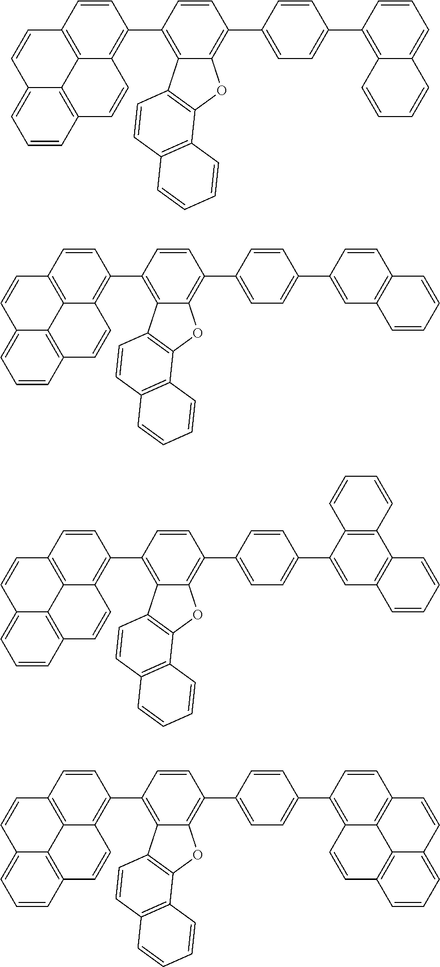

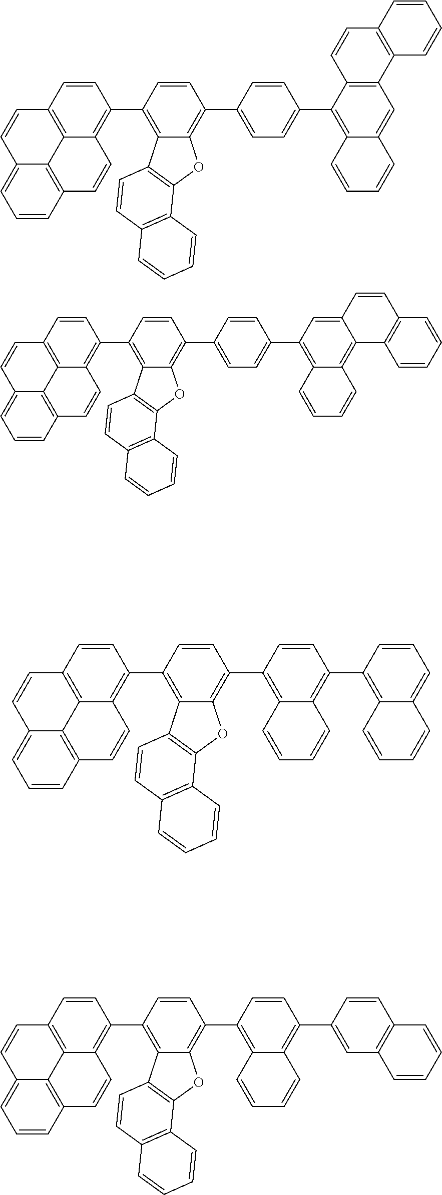

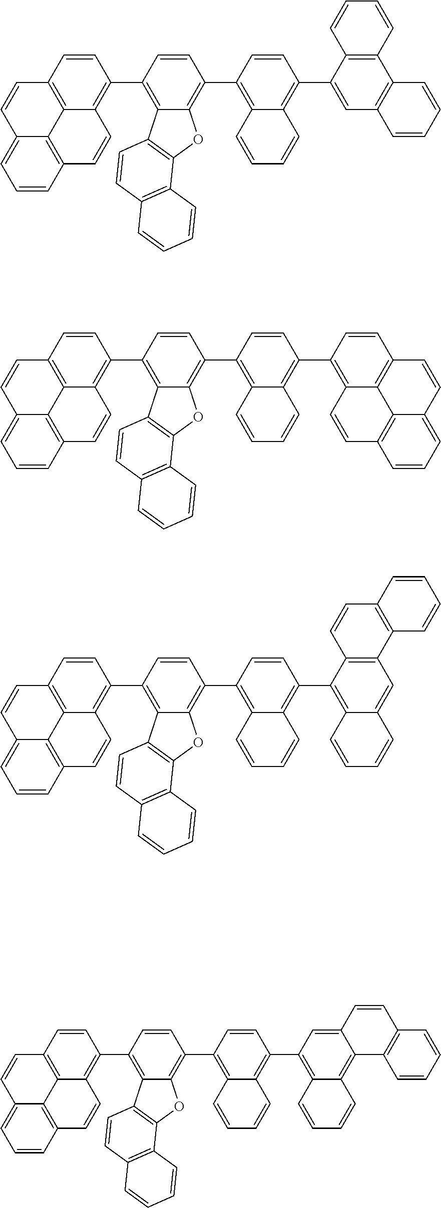 [SMILES: c1ccc2c(c1)cc(-c1ccc(-c3ccc(-c4ccc5ccc6cccc7ccc4c5c67)c4c3oc3c5ccccc5ccc34)c3ccccc13)c1ccccc12 ; c1ccc2c(c1)ccc1c2oc2c(-c3ccc(-c4ccc5ccc6cccc7ccc4c5c67)c4ccccc34)ccc(-c3ccc4ccc5cccc6ccc3c4c56)c21 ; c1ccc2c(-c3ccc(-c4ccc(-c5ccc6ccc7cccc8ccc5c6c78)c5c4oc4c6ccccc6ccc45)c4ccccc34)c3ccc4ccccc4c3cc2c1 ; c1ccc2c(c1)ccc1c2oc2c(-c3ccc(-c4cc5ccc6ccccc6c5c5ccccc45)c4ccccc34)ccc(-c3ccc4ccc5cccc6ccc3c4c56)c21]

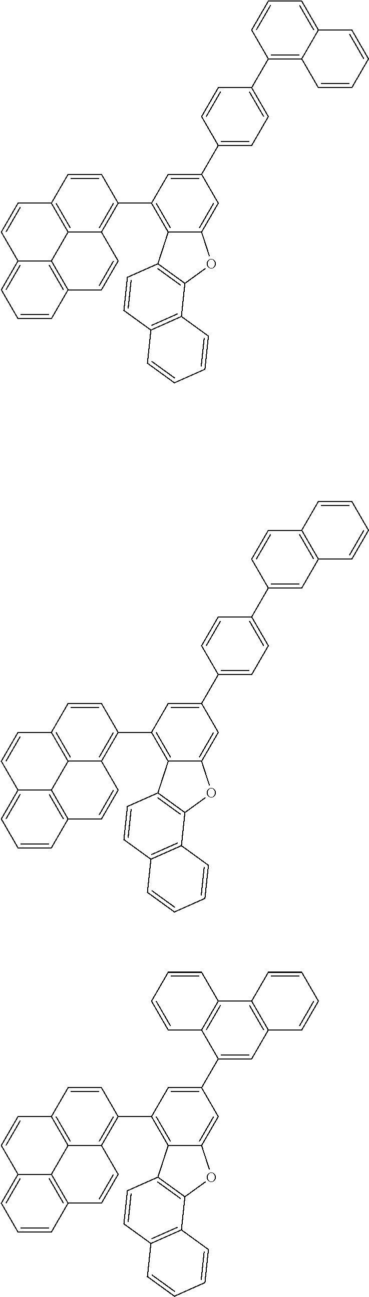 [SMILES: c1ccc2c(-c3ccc(-c4cc(-c5ccc6ccc7cccc8ccc5c6c78)c5c(c4)oc4c6ccccc6ccc45)cc3)cccc2c1 ; c1ccc2cc(-c3ccc(-c4cc(-c5ccc6ccc7cccc8ccc5c6c78)c5c(c4)oc4c6ccccc6ccc45)cc3)ccc2c1 ; c1ccc2c(c1)cc(-c1cc(-c3ccc4ccc5cccc6ccc3c4c56)c3c(c1)oc1c4ccccc4ccc13)c1ccccc12]

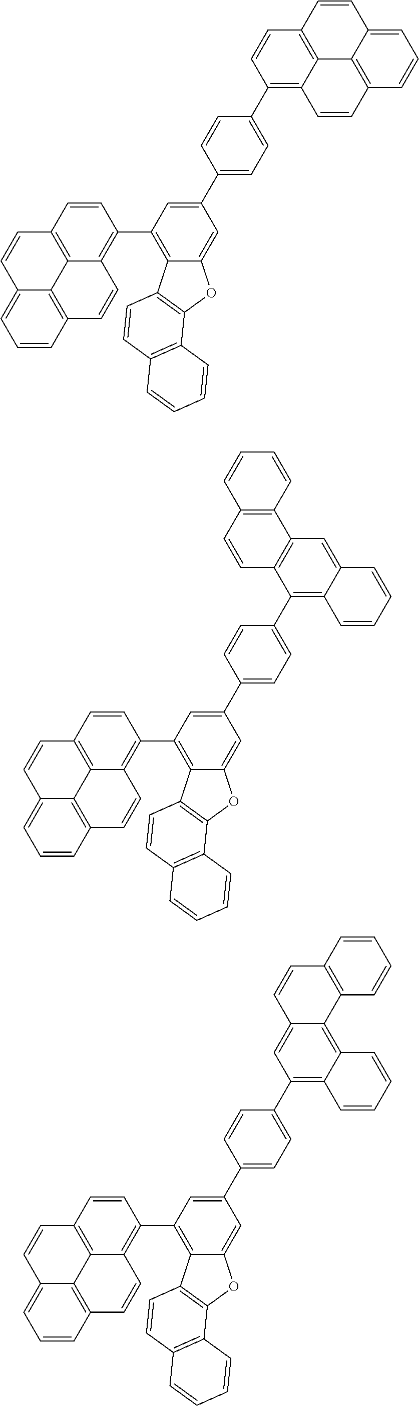 [SMILES: c1ccc2c(c1)ccc1c2oc2cc(-c3ccc(-c4ccc5ccc6cccc7ccc4c5c67)cc3)cc(-c3ccc4ccc5cccc6ccc3c4c56)c21 ; c1ccc2c(-c3ccc(-c4cc(-c5ccc6ccc7cccc8ccc5c6c78)c5c(c4)oc4c6ccccc6ccc45)cc3)c3ccc4ccccc4c3cc2c1 ; c1ccc2c(c1)ccc1c2oc2cc(-c3ccc(-c4cc5ccc6ccccc6c5c5ccccc45)cc3)cc(-c3ccc4ccc5cccc6ccc3c4c56)c21]

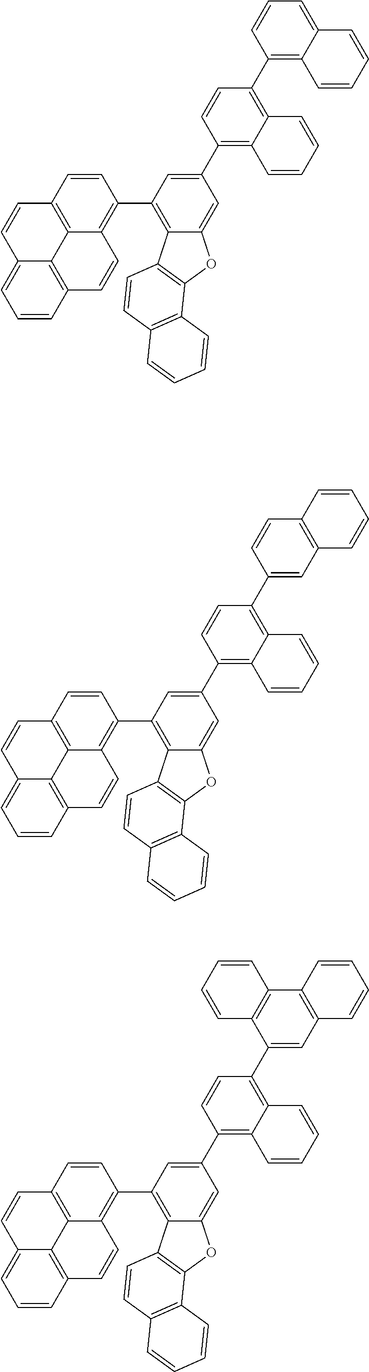 [SMILES: c1ccc2c(-c3ccc(-c4cc(-c5ccc6ccc7cccc8ccc5c6c78)c5c(c4)oc4c6ccccc6ccc45)c4ccccc34)cccc2c1 ; c1ccc2cc(-c3ccc(-c4cc(-c5ccc6ccc7cccc8ccc5c6c78)c5c(c4)oc4c6ccccc6ccc45)c4ccccc34)ccc2c1 ; c1ccc2c(c1)cc(-c1ccc(-c3cc(-c4ccc5ccc6cccc7ccc4c5c67)c4c(c3)oc3c5ccccc5ccc34)c3ccccc13)c1ccccc12]

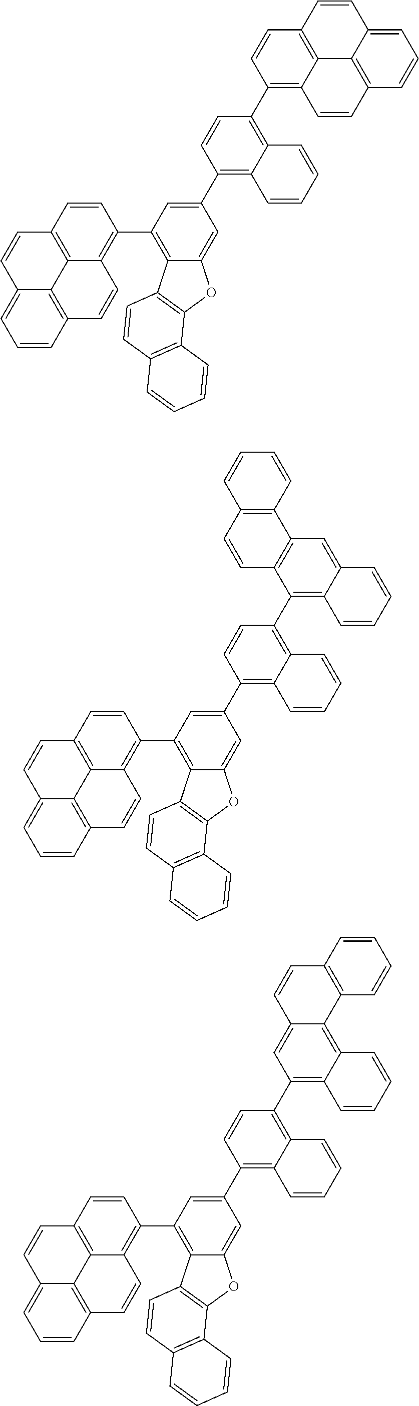 [SMILES: c1ccc2c(c1)ccc1c2oc2cc(-c3ccc(-c4ccc5ccc6cccc7ccc4c5c67)c4ccccc34)cc(-c3ccc4ccc5cccc6ccc3c4c56)c21 ; c1ccc2c(-c3ccc(-c4cc(-c5ccc6ccc7cccc8ccc5c6c78)c5c(c4)oc4c6ccccc6ccc45)c4ccccc34)c3ccc4ccccc4c3cc2c1 ; c1ccc2c(c1)ccc1c2oc2cc(-c3ccc(-c4cc5ccc6ccccc6c5c5ccccc45)c4ccccc34)cc(-c3ccc4ccc5cccc6ccc3c4c56)c21]

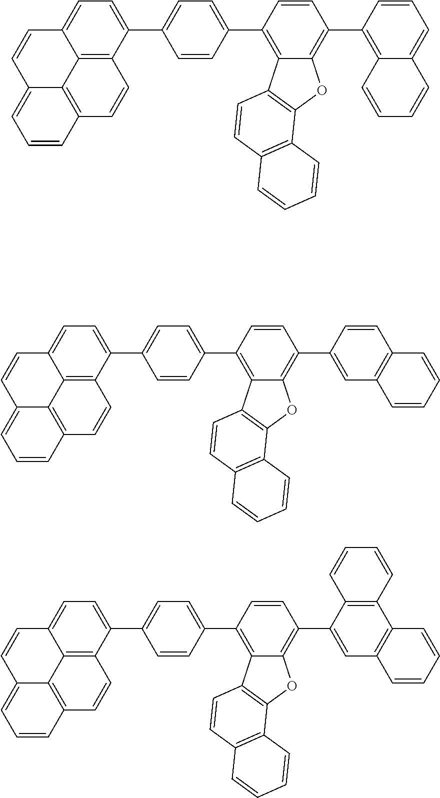 [SMILES: c1ccc2c(-c3ccc(-c4ccc(-c5ccc6ccc7cccc8ccc5c6c78)cc4)c4c3oc3c5ccccc5ccc34)cccc2c1 ; c1ccc2cc(-c3ccc(-c4ccc(-c5ccc6ccc7cccc8ccc5c6c78)cc4)c4c3oc3c5ccccc5ccc34)ccc2c1 ; c1ccc2c(c1)cc(-c1ccc(-c3ccc(-c4ccc5ccc6cccc7ccc4c5c67)cc3)c3c1oc1c4ccccc4ccc13)c1ccccc12]

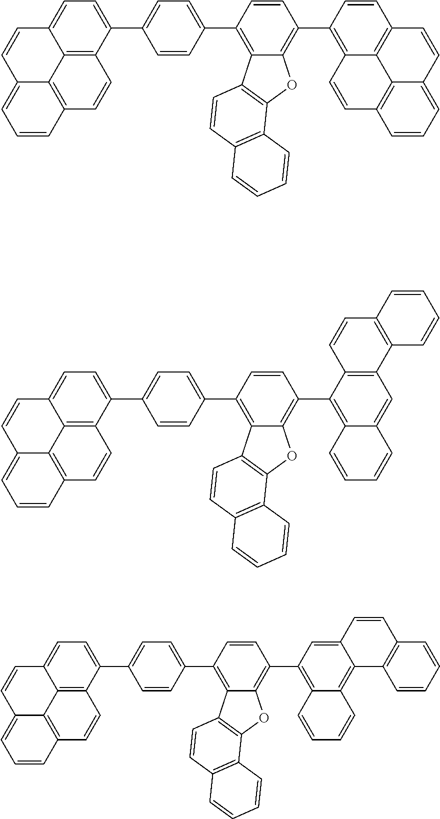 [SMILES: c1ccc2c(c1)ccc1c2oc2c(-c3ccc4ccc5cccc6ccc3c4c56)ccc(-c3ccc(-c4ccc5ccc6cccc7ccc4c5c67)cc3)c21 ; c1ccc2c(-c3ccc(-c4ccc(-c5ccc6ccc7cccc8ccc5c6c78)cc4)c4c3oc3c5ccccc5ccc34)c3ccc4ccccc4c3cc2c1 ; c1ccc2c(c1)ccc1c2oc2c(-c3cc4ccc5ccccc5c4c4ccccc34)ccc(-c3ccc(-c4ccc5ccc6cccc7ccc4c5c67)cc3)c21]

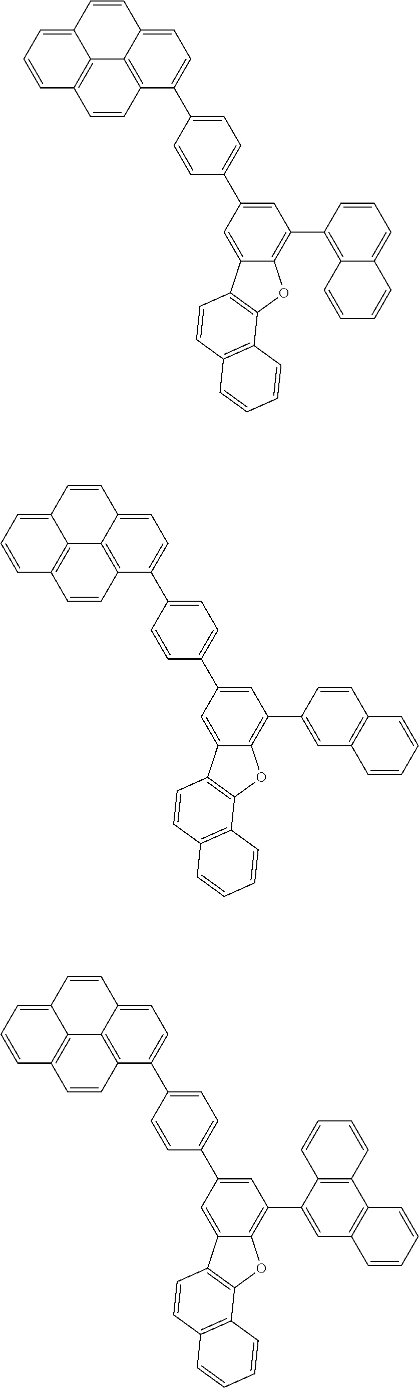 [SMILES: c1ccc2c(-c3cc(-c4ccc(-c5ccc6ccc7cccc8ccc5c6c78)cc4)cc4c3oc3c5ccccc5ccc43)cccc2c1 ; c1ccc2cc(-c3cc(-c4ccc(-c5ccc6ccc7cccc8ccc5c6c78)cc4)cc4c3oc3c5ccccc5ccc43)ccc2c1 ; c1ccc2c(c1)cc(-c1cc(-c3ccc(-c4ccc5ccc6cccc7ccc4c5c67)cc3)cc3c1oc1c4ccccc4ccc31)c1ccccc12]

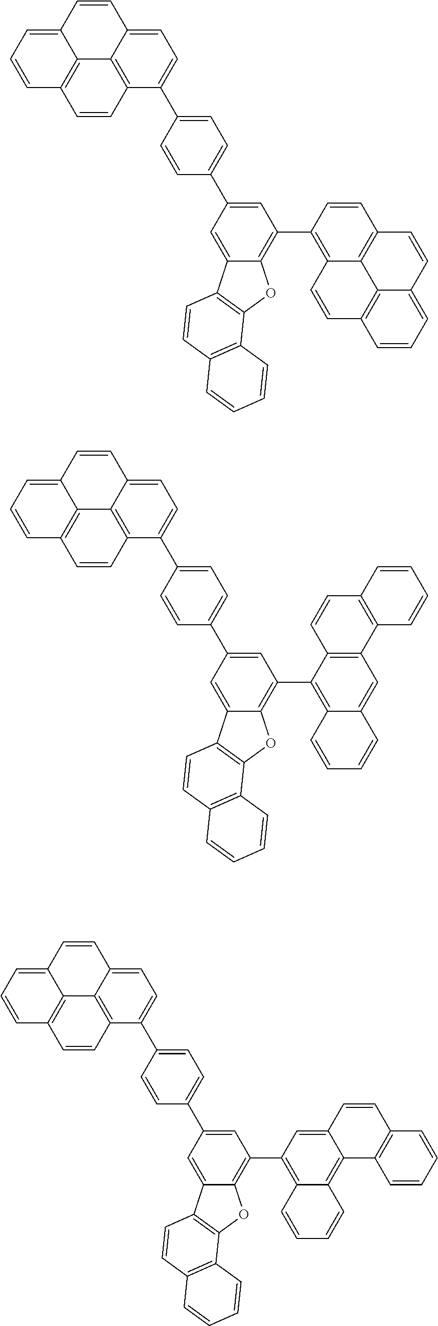 [SMILES: c1ccc2c(c1)ccc1c3cc(-c4ccc(-c5ccc6ccc7cccc8ccc5c6c78)cc4)cc(-c4ccc5ccc6cccc7ccc4c5c67)c3oc21 ; c1ccc2c(-c3cc(-c4ccc(-c5ccc6ccc7cccc8ccc5c6c78)cc4)cc4c3oc3c5ccccc5ccc43)c3ccc4ccccc4c3cc2c1 ; c1ccc2c(c1)ccc1c3cc(-c4ccc(-c5ccc6ccc7cccc8ccc5c6c78)cc4)cc(-c4cc5ccc6ccccc6c5c5ccccc45)c3oc21]

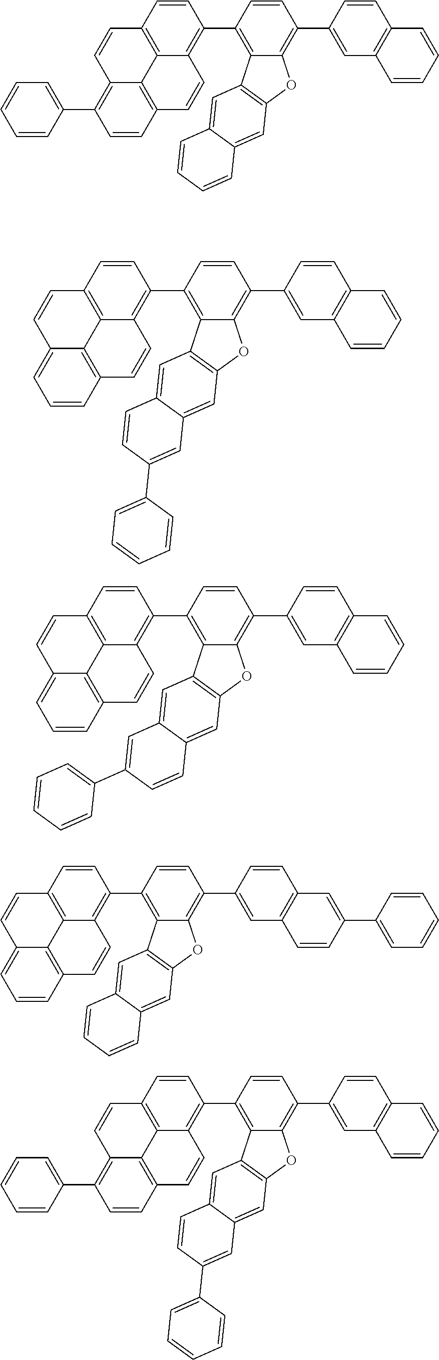

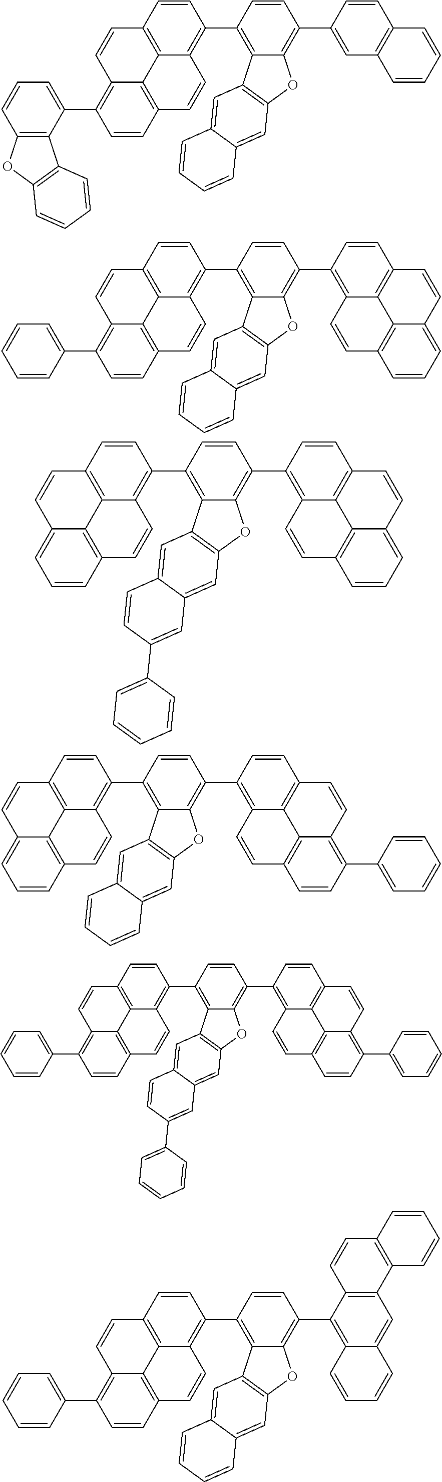

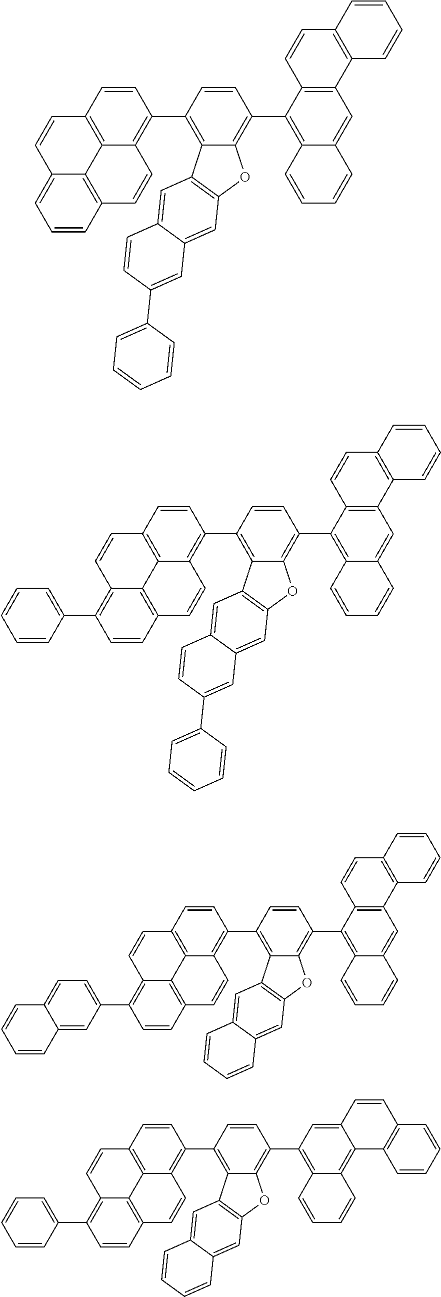 [SMILES: c1ccc(-c2ccc3cc4c(cc3c2)oc2c(-c3c5ccccc5cc5c3ccc3ccccc35)ccc(-c3ccc5ccc6cccc7ccc3c5c67)c24)cc1 ; c1ccc(-c2ccc3cc4c(cc3c2)oc2c(-c3c5ccccc5cc5c3ccc3ccccc35)ccc(-c3ccc5ccc6c(-c7ccccc7)ccc7ccc3c5c76)c24)cc1 ; c1ccc2cc(-c3ccc4ccc5c(-c6ccc(-c7c8ccccc8cc8c7ccc7ccccc78)c7oc8cc9ccccc9cc8c67)ccc6ccc3c4c65)ccc2c1 ; c1ccc(-c2ccc3ccc4c(-c5ccc(-c6cc7ccc8ccccc8c7c7ccccc67)c6oc7cc8ccccc8cc7c56)ccc5ccc2c3c54)cc1]

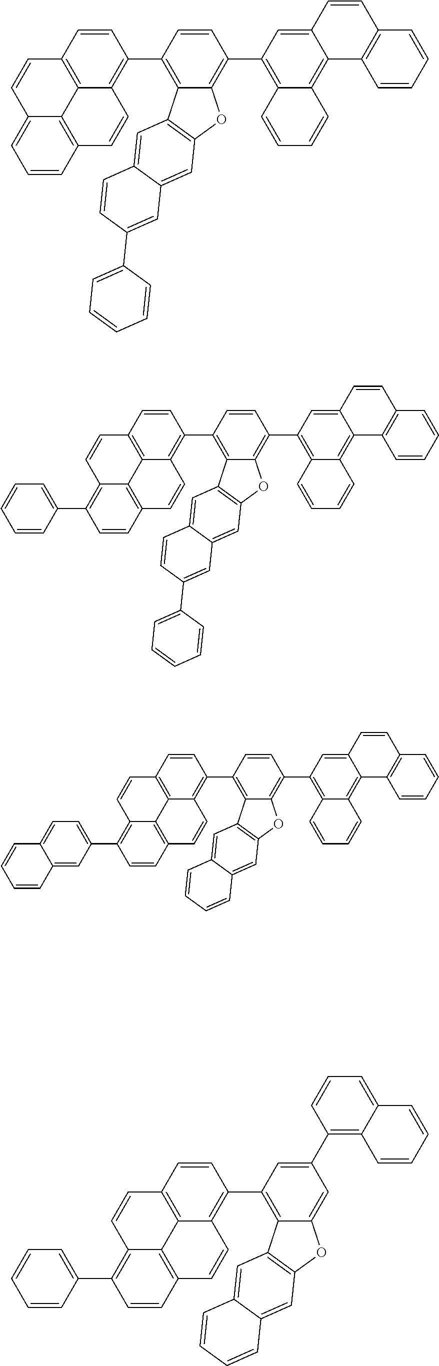 [SMILES: c1ccc(-c2ccc3cc4c(cc3c2)oc2c(-c3cc5ccc6ccccc6c5c5ccccc35)ccc(-c3ccc5ccc6cccc7ccc3c5c67)c24)cc1 ; c1ccc(-c2ccc3cc4c(cc3c2)oc2c(-c3cc5ccc6ccccc6c5c5ccccc35)ccc(-c3ccc5ccc6c(-c7ccccc7)ccc7ccc3c5c76)c24)cc1 ; c1ccc2cc(-c3ccc4ccc5c(-c6ccc(-c7cc8ccc9ccccc9c8c8ccccc78)c7oc8cc9ccccc9cc8c67)ccc6ccc3c4c65)ccc2c1 ; c1ccc(-c2ccc3ccc4c(-c5cc(-c6cccc7ccccc67)cc6oc7cc8ccccc8cc7c56)ccc5ccc2c3c54)cc1]

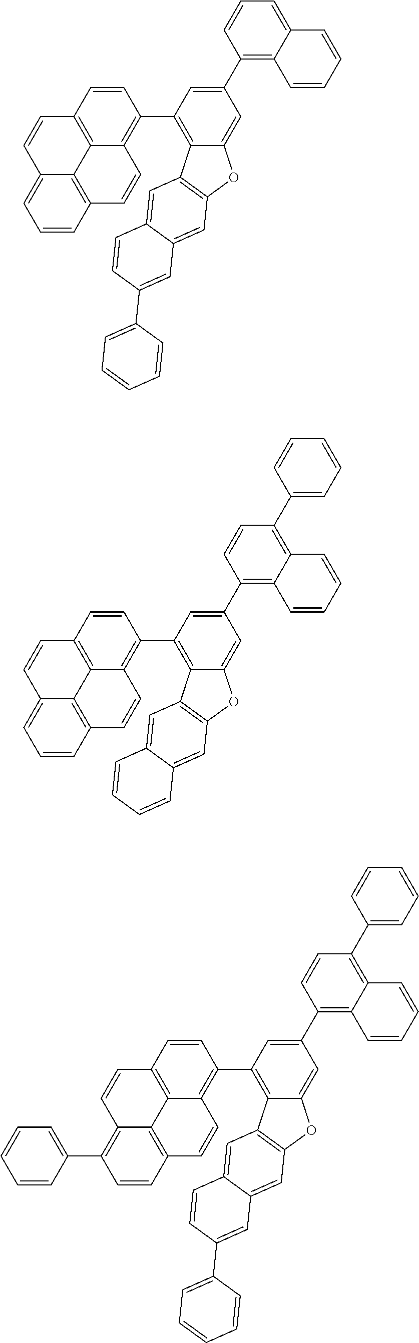 [SMILES: c1ccc(-c2ccc3cc4c(cc3c2)oc2cc(-c3cccc5ccccc35)cc(-c3ccc5ccc6cccc7ccc3c5c67)c24)cc1 ; c1ccc(-c2ccc(-c3cc(-c4ccc5ccc6cccc7ccc4c5c67)c4c(c3)oc3cc5ccccc5cc34)c3ccccc23)cc1 ; c1ccc(-c2ccc3cc4c(cc3c2)oc2cc(-c3ccc(-c5ccccc5)c5ccccc35)cc(-c3ccc5ccc6c(-c7ccccc7)ccc7ccc3c5c76)c24)cc1]

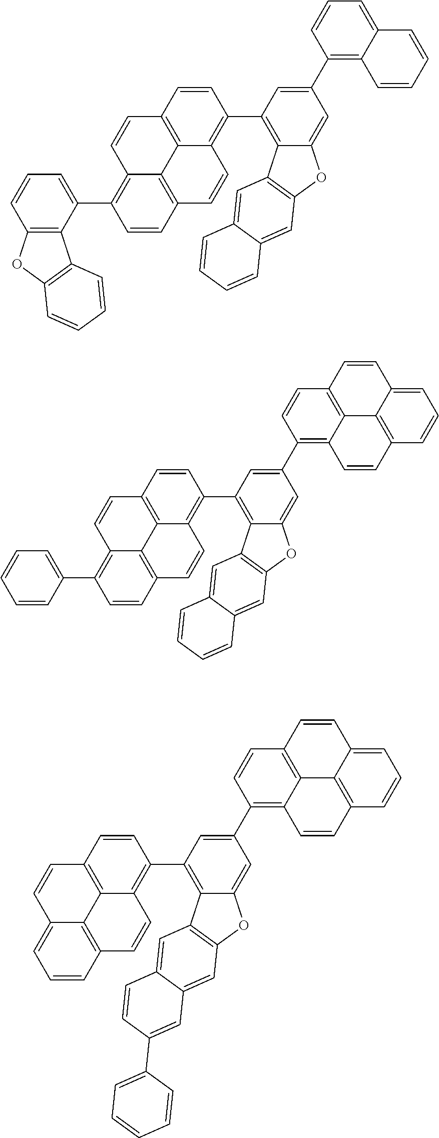 [SMILES: c1ccc2cc3c(cc2c1)oc1cc(-c2cccc4ccccc24)cc(-c2ccc4ccc5c(-c6cccc7oc8ccccc8c67)ccc6ccc2c4c65)c13 ; c1ccc(-c2ccc3ccc4c(-c5cc(-c6ccc7ccc8cccc9ccc6c7c89)cc6oc7cc8ccccc8cc7c56)ccc5ccc2c3c54)cc1 ; c1ccc(-c2ccc3cc4c(cc3c2)oc2cc(-c3ccc5ccc6cccc7ccc3c5c67)cc(-c3ccc5ccc6cccc7ccc3c5c67)c24)cc1]

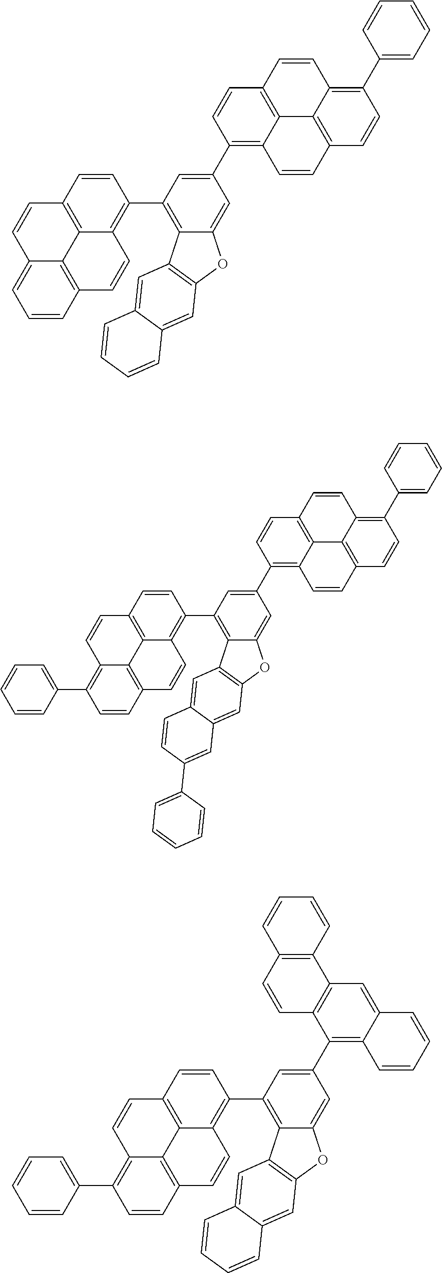 [SMILES: c1ccc(-c2ccc3ccc4c(-c5cc(-c6ccc7ccc8cccc9ccc6c7c89)c6c(c5)oc5cc7ccccc7cc56)ccc5ccc2c3c54)cc1 ; c1ccc(-c2ccc3cc4c(cc3c2)oc2cc(-c3ccc5ccc6c(-c7ccccc7)ccc7ccc3c5c76)cc(-c3ccc5ccc6c(-c7ccccc7)ccc7ccc3c5c76)c24)cc1 ; c1ccc(-c2ccc3ccc4c(-c5cc(-c6c7ccccc7cc7c6ccc6ccccc67)cc6oc7cc8ccccc8cc7c56)ccc5ccc2c3c54)cc1]

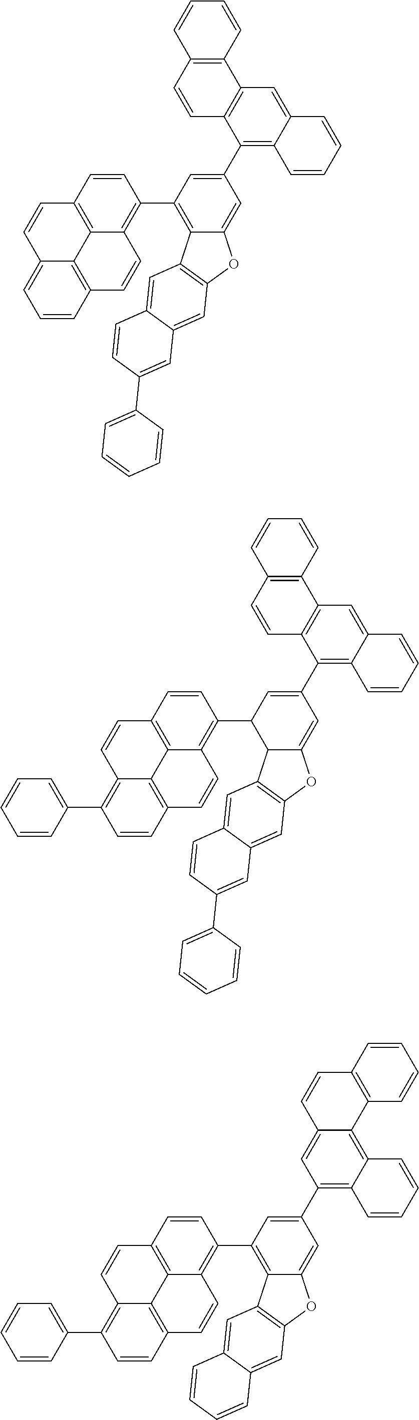 [SMILES: c1ccc(-c2ccc3cc4c(cc3c2)oc2cc(-c3c5ccccc5cc5c3ccc3ccccc35)cc(-c3ccc5ccc6cccc7ccc3c5c67)c24)cc1 ; C1=C(c2c3ccccc3cc3c2ccc2ccccc23)C=C2Oc3cc4cc(-c5ccccc5)ccc4cc3C2C1c1ccc2ccc3c(-c4ccccc4)ccc4ccc1c2c43 ; c1ccc(-c2ccc3ccc4c(-c5cc(-c6cc7ccc8ccccc8c7c7ccccc67)cc6oc7cc8ccccc8cc7c56)ccc5ccc2c3c54)cc1]

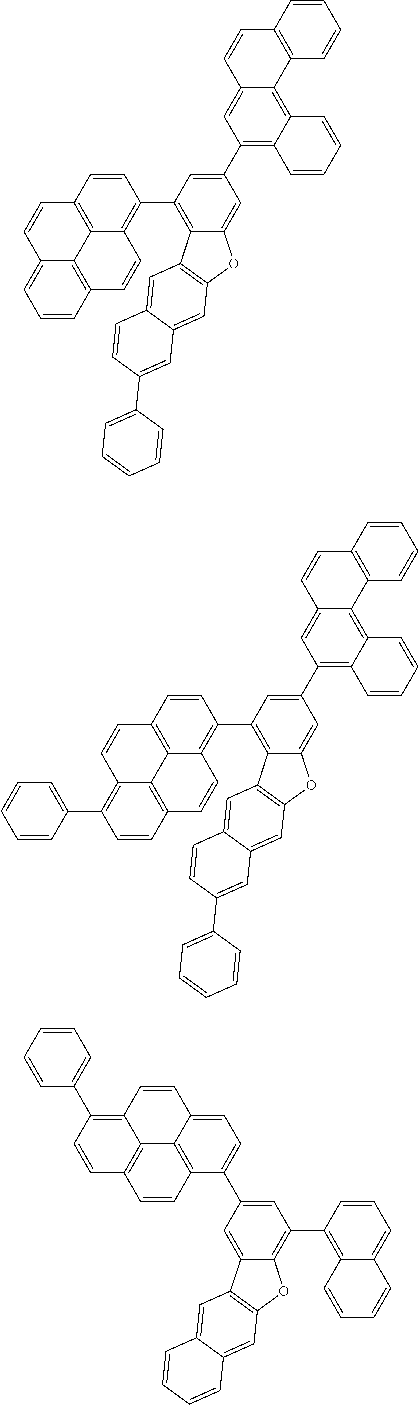 [SMILES: c1ccc(-c2ccc3cc4c(cc3c2)oc2cc(-c3cc5ccc6ccccc6c5c5ccccc35)cc(-c3ccc5ccc6cccc7ccc3c5c67)c24)cc1 ; c1ccc(-c2ccc3cc4c(cc3c2)oc2cc(-c3cc5ccc6ccccc6c5c5ccccc35)cc(-c3ccc5ccc6c(-c7ccccc7)ccc7ccc3c5c76)c24)cc1 ; c1ccc(-c2ccc3ccc4c(-c5cc(-c6cccc7ccccc67)c6oc7cc8ccccc8cc7c6c5)ccc5ccc2c3c54)cc1]

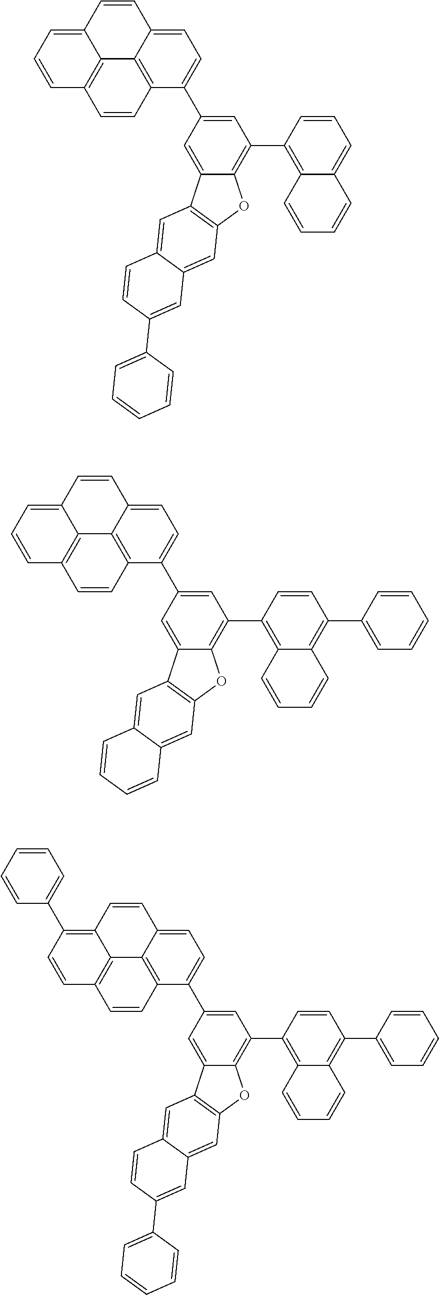 [SMILES: c1ccc(-c2ccc3cc4c(cc3c2)oc2c(-c3cccc5ccccc35)cc(-c3ccc5ccc6cccc7ccc3c5c67)cc24)cc1 ; c1ccc(-c2ccc(-c3cc(-c4ccc5ccc6cccc7ccc4c5c67)cc4c3oc3cc5ccccc5cc34)c3ccccc23)cc1 ; c1ccc(-c2ccc3cc4c(cc3c2)oc2c(-c3ccc(-c5ccccc5)c5ccccc35)cc(-c3ccc5ccc6c(-c7ccccc7)ccc7ccc3c5c76)cc24)cc1]

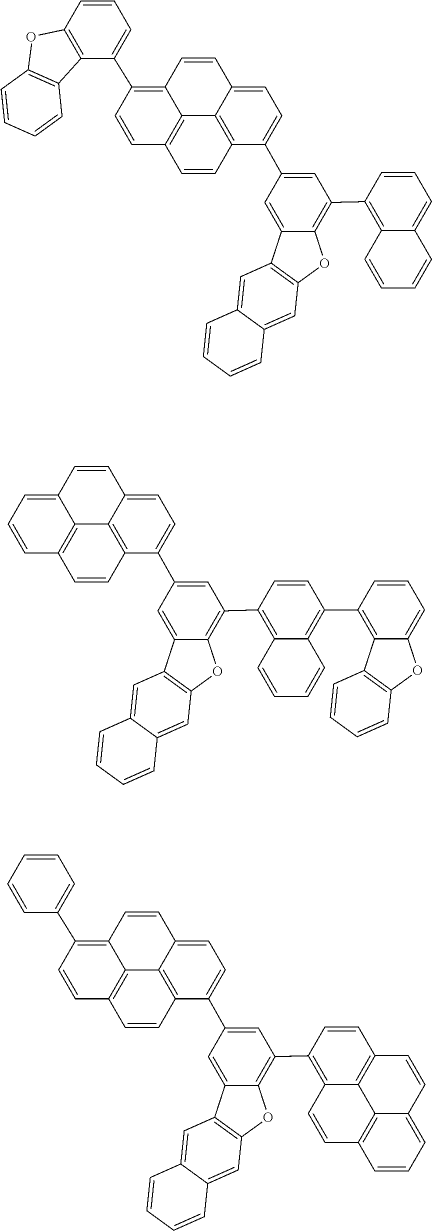 [SMILES: c1ccc2cc3c(cc2c1)oc1c(-c2cccc4ccccc24)cc(-c2ccc4ccc5c(-c6cccc7oc8ccccc8c67)ccc6ccc2c4c65)cc13 ; c1ccc2cc3c(cc2c1)oc1c(-c2ccc(-c4cccc5oc6ccccc6c45)c4ccccc24)cc(-c2ccc4ccc5cccc6ccc2c4c56)cc13 ; c1ccc(-c2ccc3ccc4c(-c5cc(-c6ccc7ccc8cccc9ccc6c7c89)c6oc7cc8ccccc8cc7c6c5)ccc5ccc2c3c54)cc1]

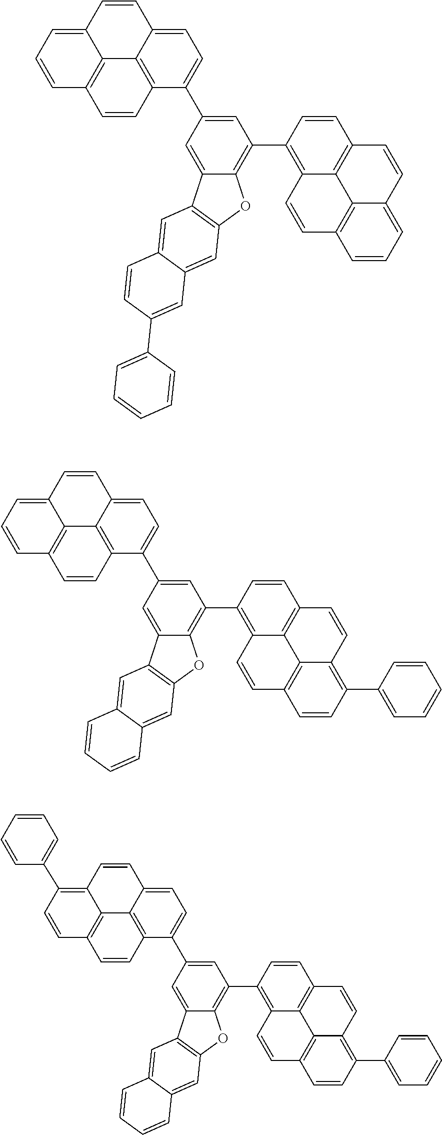 [SMILES: c1ccc(-c2ccc3cc4c(cc3c2)oc2c(-c3ccc5ccc6cccc7ccc3c5c67)cc(-c3ccc5ccc6cccc7ccc3c5c67)cc24)cc1 ; c1ccc(-c2ccc3ccc4c(-c5cc(-c6ccc7ccc8cccc9ccc6c7c89)cc6c5oc5cc7ccccc7cc56)ccc5ccc2c3c54)cc1 ; c1ccc(-c2ccc3ccc4c(-c5cc(-c6ccc7ccc8c(-c9ccccc9)ccc9ccc6c7c98)c6oc7cc8ccccc8cc7c6c5)ccc5ccc2c3c54)cc1]

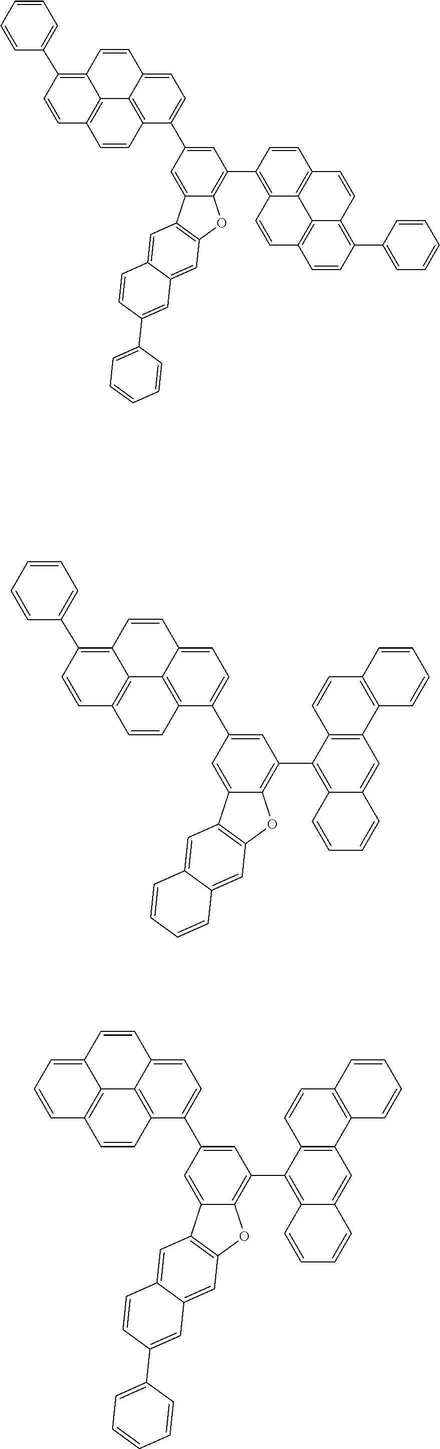 [SMILES: c1ccc(-c2ccc3cc4c(cc3c2)oc2c(-c3ccc5ccc6c(-c7ccccc7)ccc7ccc3c5c76)cc(-c3ccc5ccc6c(-c7ccccc7)ccc7ccc3c5c76)cc24)cc1 ; c1ccc(-c2ccc3ccc4c(-c5cc(-c6c7ccccc7cc7c6ccc6ccccc67)c6oc7cc8ccccc8cc7c6c5)ccc5ccc2c3c54)cc1 ; c1ccc(-c2ccc3cc4c(cc3c2)oc2c(-c3c5ccccc5cc5c3ccc3ccccc35)cc(-c3ccc5ccc6cccc7ccc3c5c67)cc24)cc1]

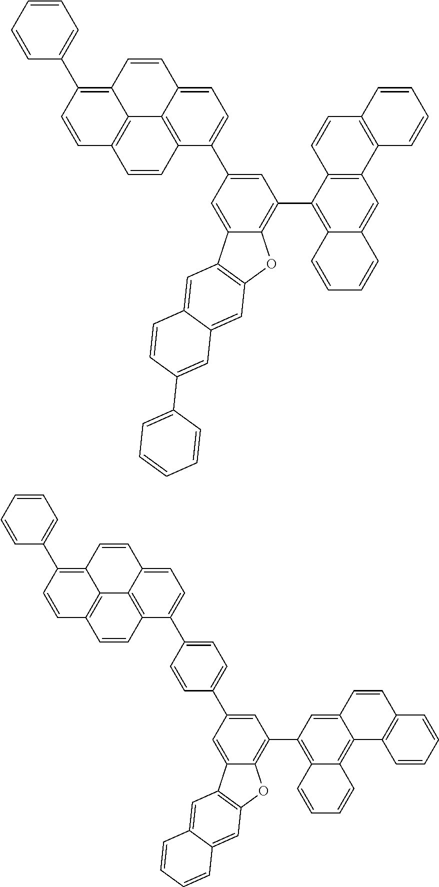 [SMILES: c1ccc(-c2ccc3cc4c(cc3c2)oc2c(-c3c5ccccc5cc5c3ccc3ccccc35)cc(-c3ccc5ccc6c(-c7ccccc7)ccc7ccc3c5c76)cc24)cc1 ; c1ccc(-c2ccc3ccc4c(-c5ccc(-c6cc(-c7cc8ccc9ccccc9c8c8ccccc78)c7oc8cc9ccccc9cc8c7c6)cc5)ccc5ccc2c3c54)cc1]

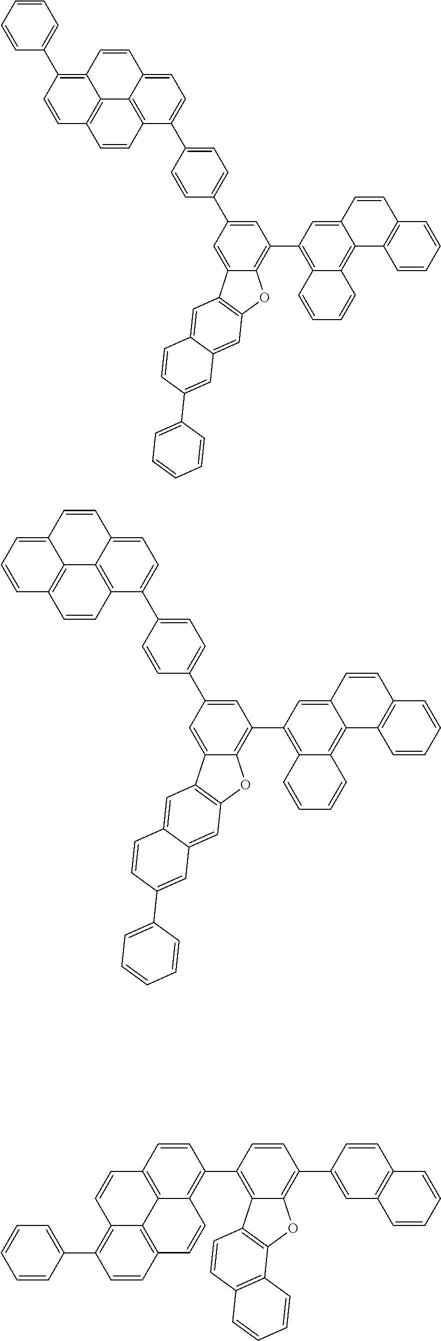 [SMILES: c1ccc(-c2ccc3cc4c(cc3c2)oc2c(-c3cc5ccc6ccccc6c5c5ccccc35)cc(-c3ccc(-c5ccc6ccc7c(-c8ccccc8)ccc8ccc5c6c87)cc3)cc24)cc1 ; c1ccc(-c2ccc3cc4c(cc3c2)oc2c(-c3cc5ccc6ccccc6c5c5ccccc35)cc(-c3ccc(-c5ccc6ccc7cccc8ccc5c6c78)cc3)cc24)cc1 ; c1ccc(-c2ccc3ccc4c(-c5ccc(-c6ccc7ccccc7c6)c6oc7c8ccccc8ccc7c56)ccc5ccc2c3c54)cc1]

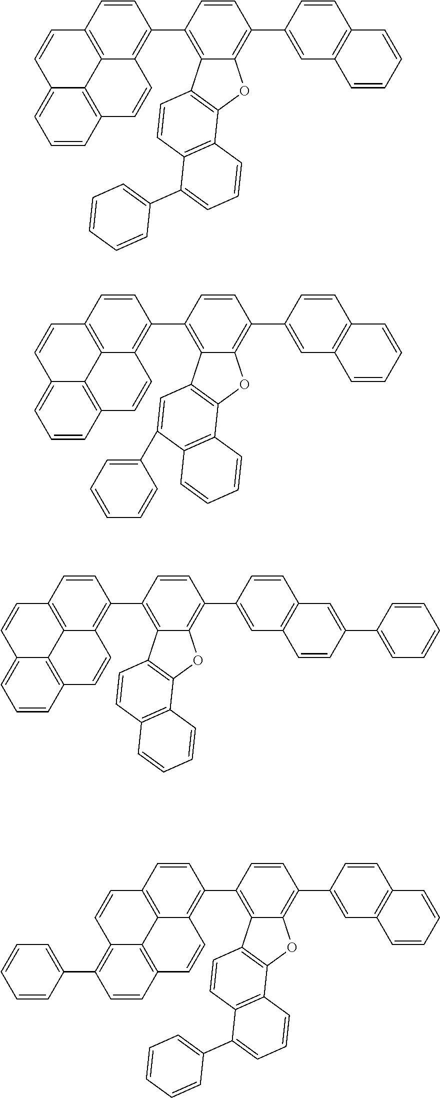 [SMILES: c1ccc(-c2cccc3c2ccc2c3oc3c(-c4ccc5ccccc5c4)ccc(-c4ccc5ccc6cccc7ccc4c5c67)c32)cc1 ; c1ccc(-c2cc3c(oc4c(-c5ccc6ccccc6c5)ccc(-c5ccc6ccc7cccc8ccc5c6c78)c43)c3ccccc23)cc1 ; c1ccc(-c2ccc3cc(-c4ccc(-c5ccc6ccc7cccc8ccc5c6c78)c5c4oc4c6ccccc6ccc45)ccc3c2)cc1 ; c1ccc(-c2cccc3c2ccc2c3oc3c(-c4ccc5ccccc5c4)ccc(-c4ccc5ccc6c(-c7ccccc7)ccc7ccc4c5c76)c32)cc1]

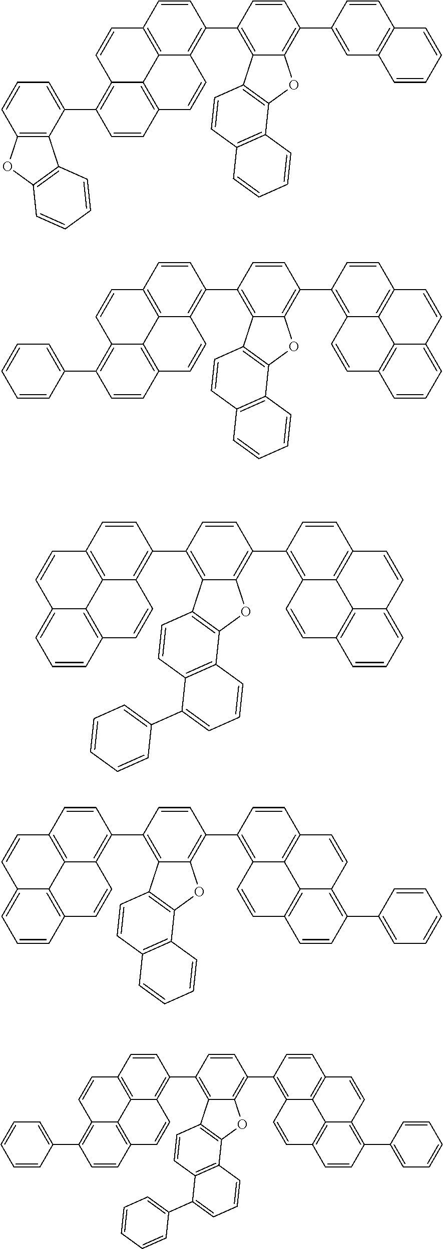

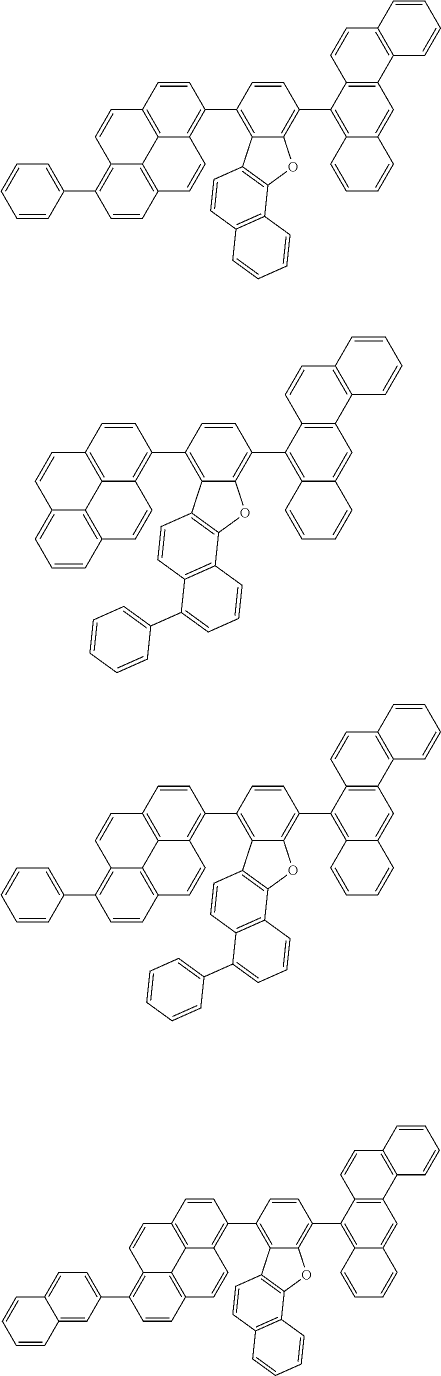 [SMILES: c1ccc(-c2ccc3ccc4c(-c5ccc(-c6c7ccccc7cc7c6ccc6ccccc67)c6oc7c8ccccc8ccc7c56)ccc5ccc2c3c54)cc1 ; c1ccc(-c2cccc3c2ccc2c3oc3c(-c4c5ccccc5cc5c4ccc4ccccc45)ccc(-c4ccc5ccc6cccc7ccc4c5c67)c32)cc1 ; c1ccc(-c2cccc3c2ccc2c3oc3c(-c4c5ccccc5cc5c4ccc4ccccc45)ccc(-c4ccc5ccc6c(-c7ccccc7)ccc7ccc4c5c76)c32)cc1 ; c1ccc2cc(-c3ccc4ccc5c(-c6ccc(-c7c8ccccc8cc8c7ccc7ccccc78)c7oc8c9ccccc9ccc8c67)ccc6ccc3c4c65)ccc2c1]

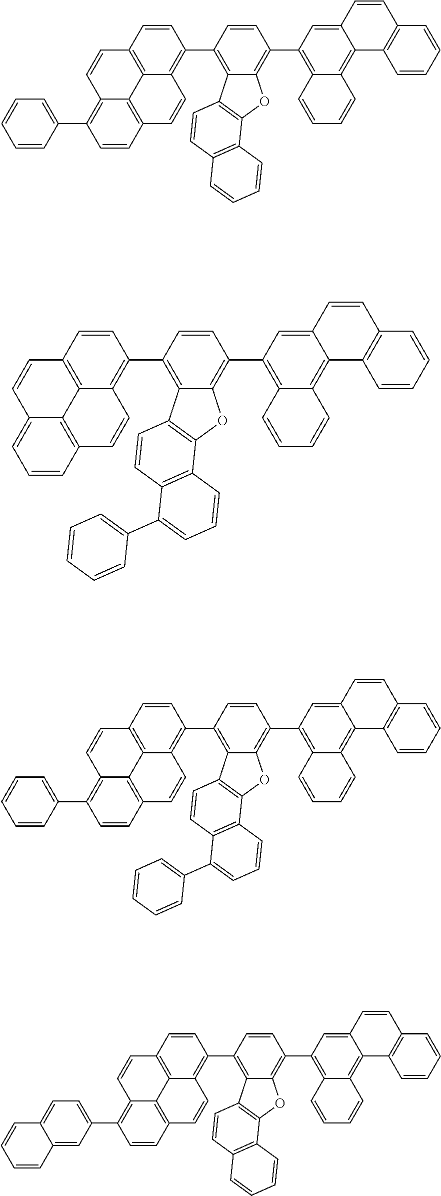 [SMILES: c1ccc(-c2ccc3ccc4c(-c5ccc(-c6cc7ccc8ccccc8c7c7ccccc67)c6oc7c8ccccc8ccc7c56)ccc5ccc2c3c54)cc1 ; c1ccc(-c2cccc3c2ccc2c3oc3c(-c4cc5ccc6ccccc6c5c5ccccc45)ccc(-c4ccc5ccc6cccc7ccc4c5c67)c32)cc1 ; c1ccc(-c2cccc3c2ccc2c3oc3c(-c4cc5ccc6ccccc6c5c5ccccc45)ccc(-c4ccc5ccc6c(-c7ccccc7)ccc7ccc4c5c76)c32)cc1 ; c1ccc2cc(-c3ccc4ccc5c(-c6ccc(-c7cc8ccc9ccccc9c8c8ccccc78)c7oc8c9ccccc9ccc8c67)ccc6ccc3c4c65)ccc2c1]

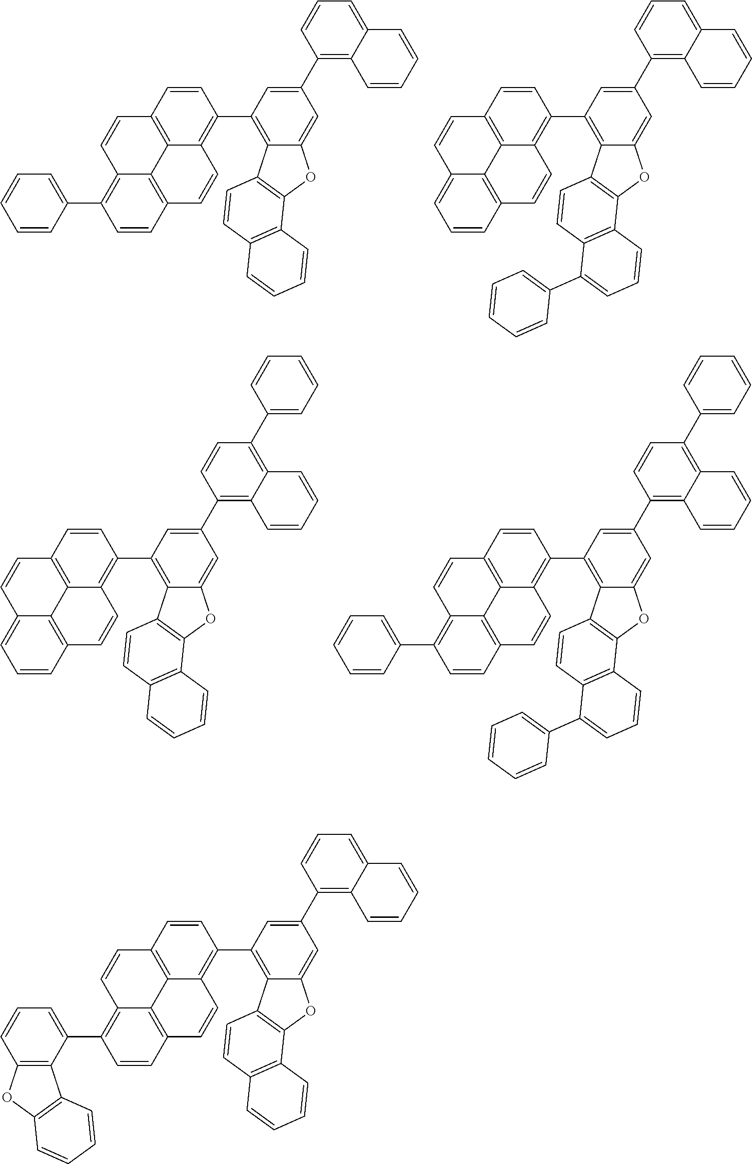 [SMILES: c1ccc(-c2ccc3ccc4c(-c5cc(-c6cccc7ccccc67)cc6oc7c8ccccc8ccc7c56)ccc5ccc2c3c54)cc1 ; c1ccc(-c2cccc3c2ccc2c3oc3cc(-c4cccc5ccccc45)cc(-c4ccc5ccc6cccc7ccc4c5c67)c32)cc1 ; c1ccc(-c2ccc(-c3cc(-c4ccc5ccc6cccc7ccc4c5c67)c4c(c3)oc3c5ccccc5ccc34)c3ccccc23)cc1 ; c1ccc(-c2ccc(-c3cc(-c4ccc5ccc6c(-c7ccccc7)ccc7ccc4c5c76)c4c(c3)oc3c5cccc(-c6ccccc6)c5ccc34)c3ccccc23)cc1 ; c1ccc2c(-c3cc(-c4ccc5ccc6c(-c7cccc8oc9ccccc9c78)ccc7ccc4c5c76)c4c(c3)oc3c5ccccc5ccc34)cccc2c1]

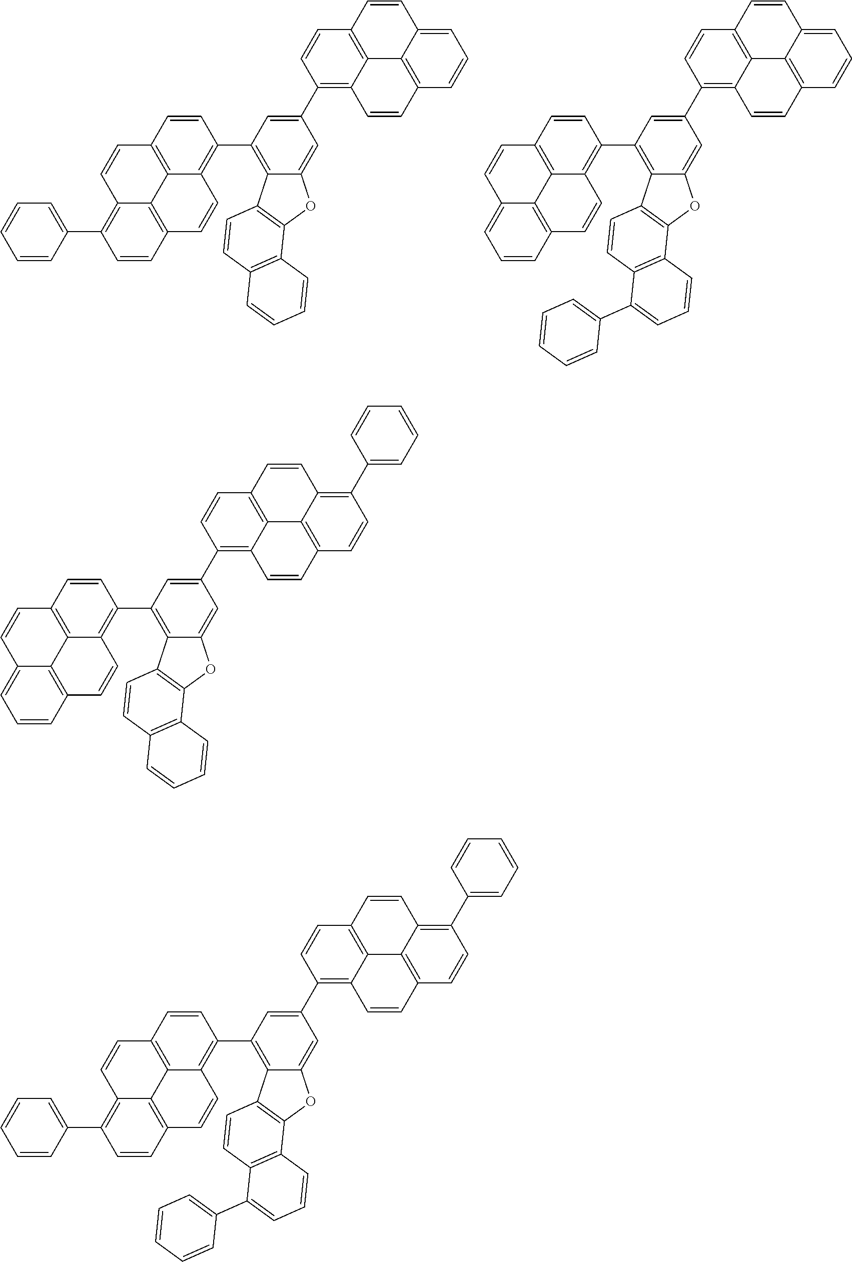 [SMILES: c1ccc(-c2ccc3ccc4c(-c5cc(-c6ccc7ccc8cccc9ccc6c7c89)cc6oc7c8ccccc8ccc7c56)ccc5ccc2c3c54)cc1 ; c1ccc(-c2cccc3c2ccc2c3oc3cc(-c4ccc5ccc6cccc7ccc4c5c67)cc(-c4ccc5ccc6cccc7ccc4c5c67)c32)cc1 ; c1ccc(-c2ccc3ccc4c(-c5cc(-c6ccc7ccc8cccc9ccc6c7c89)c6c(c5)oc5c7ccccc7ccc56)ccc5ccc2c3c54)cc1 ; c1ccc(-c2cccc3c2ccc2c3oc3cc(-c4ccc5ccc6c(-c7ccccc7)ccc7ccc4c5c76)cc(-c4ccc5ccc6c(-c7ccccc7)ccc7ccc4c5c76)c32)cc1]

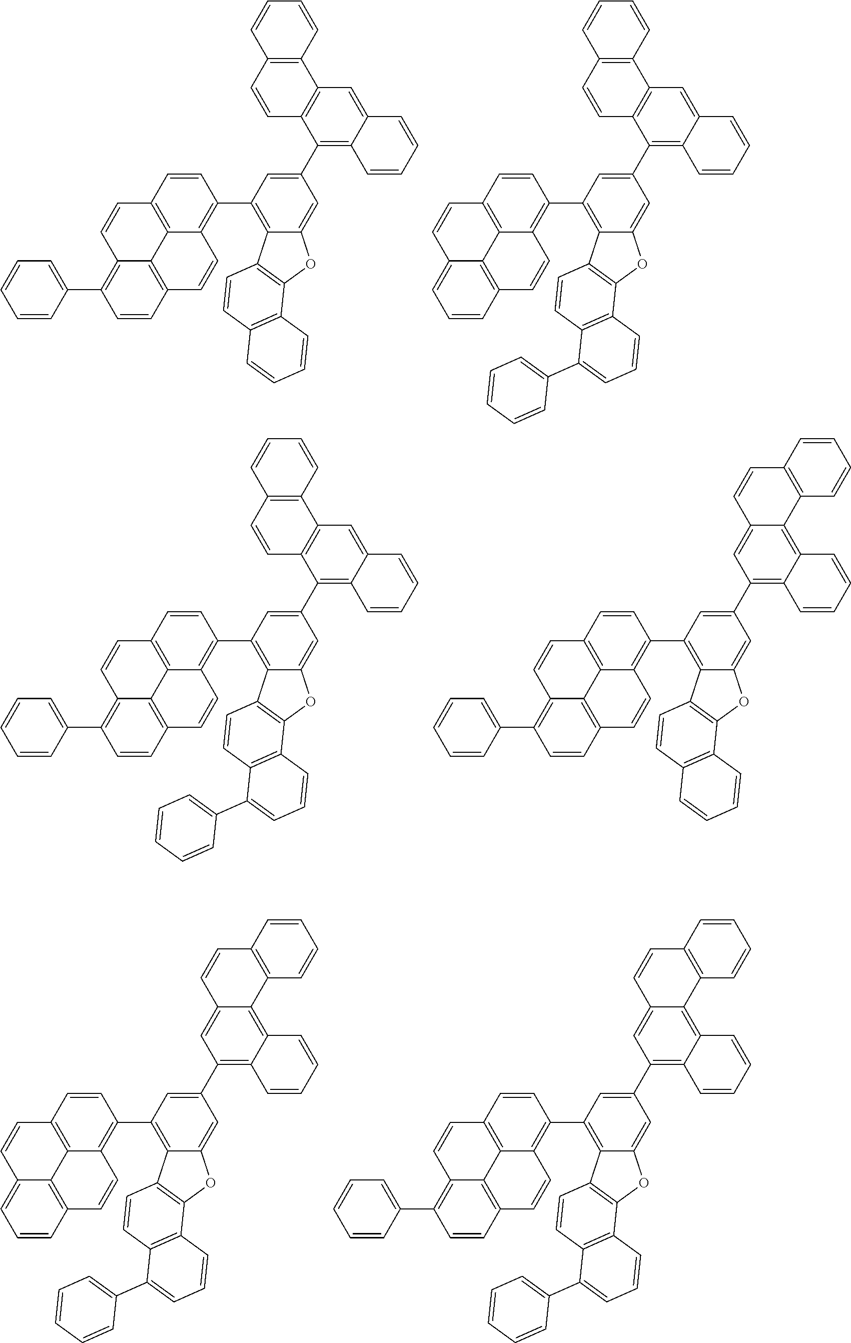

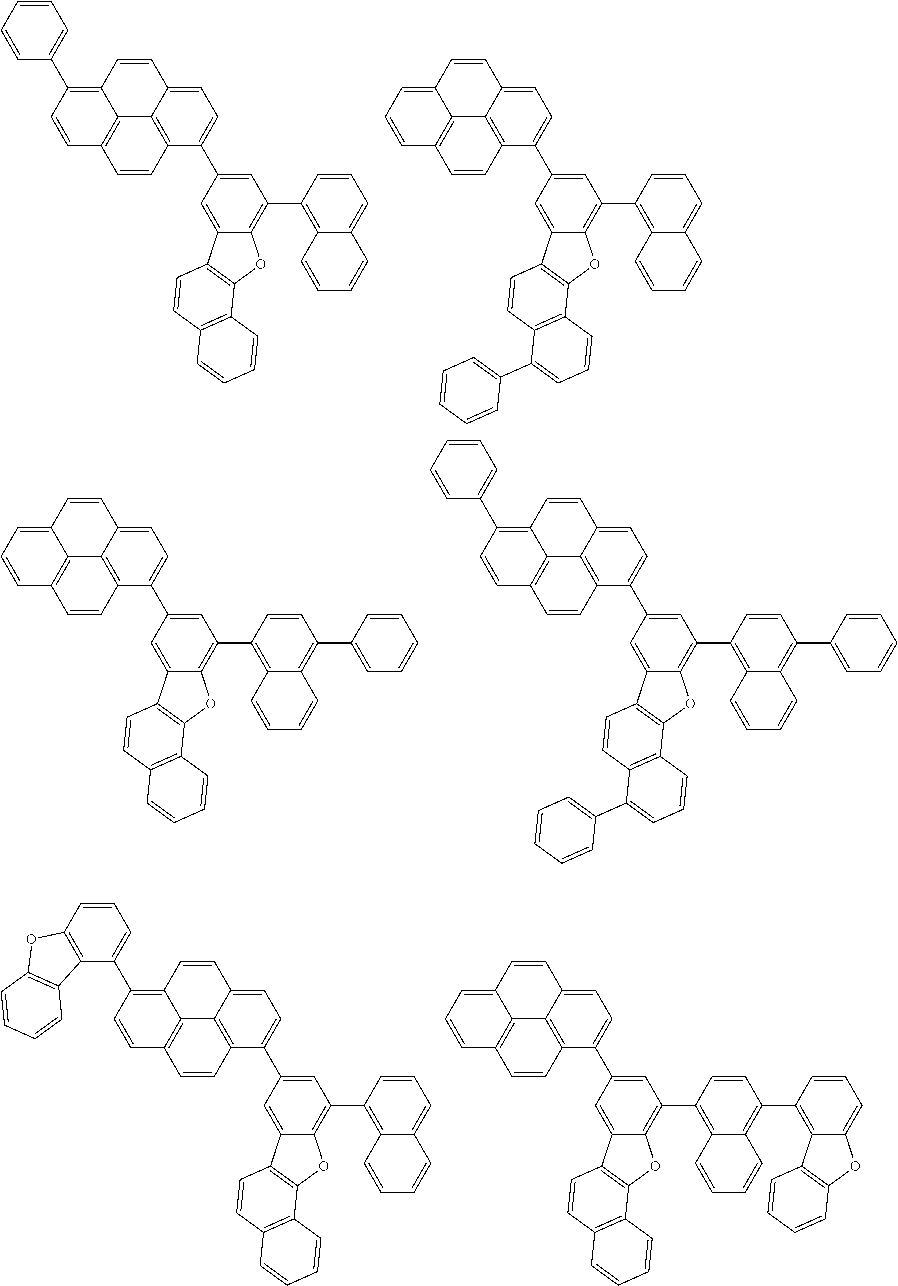

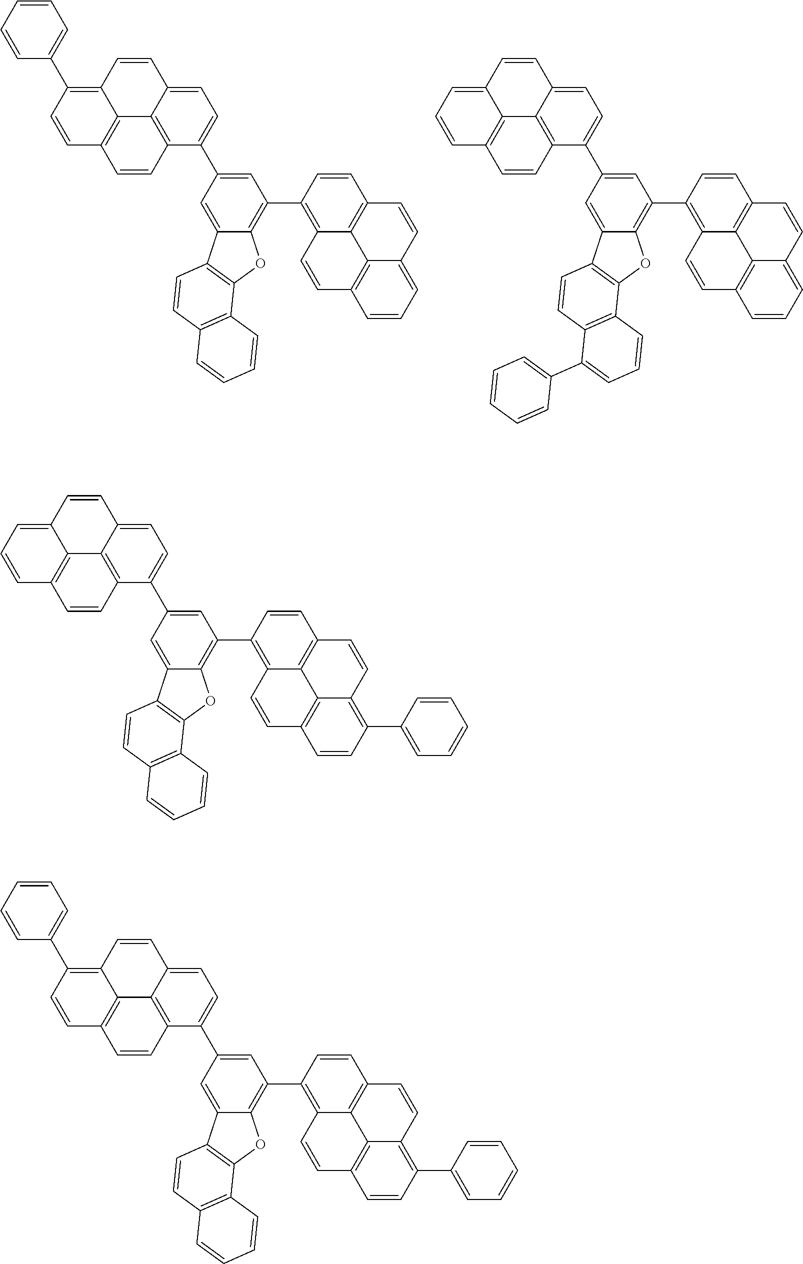 [SMILES: c1ccc(-c2ccc3ccc4c(-c5cc(-c6ccc7ccc8cccc9ccc6c7c89)c6oc7c8ccccc8ccc7c6c5)ccc5ccc2c3c54)cc1 ; c1ccc(-c2cccc3c2ccc2c4cc(-c5ccc6ccc7cccc8ccc5c6c78)cc(-c5ccc6ccc7cccc8ccc5c6c78)c4oc32)cc1 ; c1ccc(-c2ccc3ccc4c(-c5cc(-c6ccc7ccc8cccc9ccc6c7c89)cc6c5oc5c7ccccc7ccc65)ccc5ccc2c3c54)cc1 ; c1ccc(-c2ccc3ccc4c(-c5cc(-c6ccc7ccc8c(-c9ccccc9)ccc9ccc6c7c98)c6oc7c8ccccc8ccc7c6c5)ccc5ccc2c3c54)cc1]

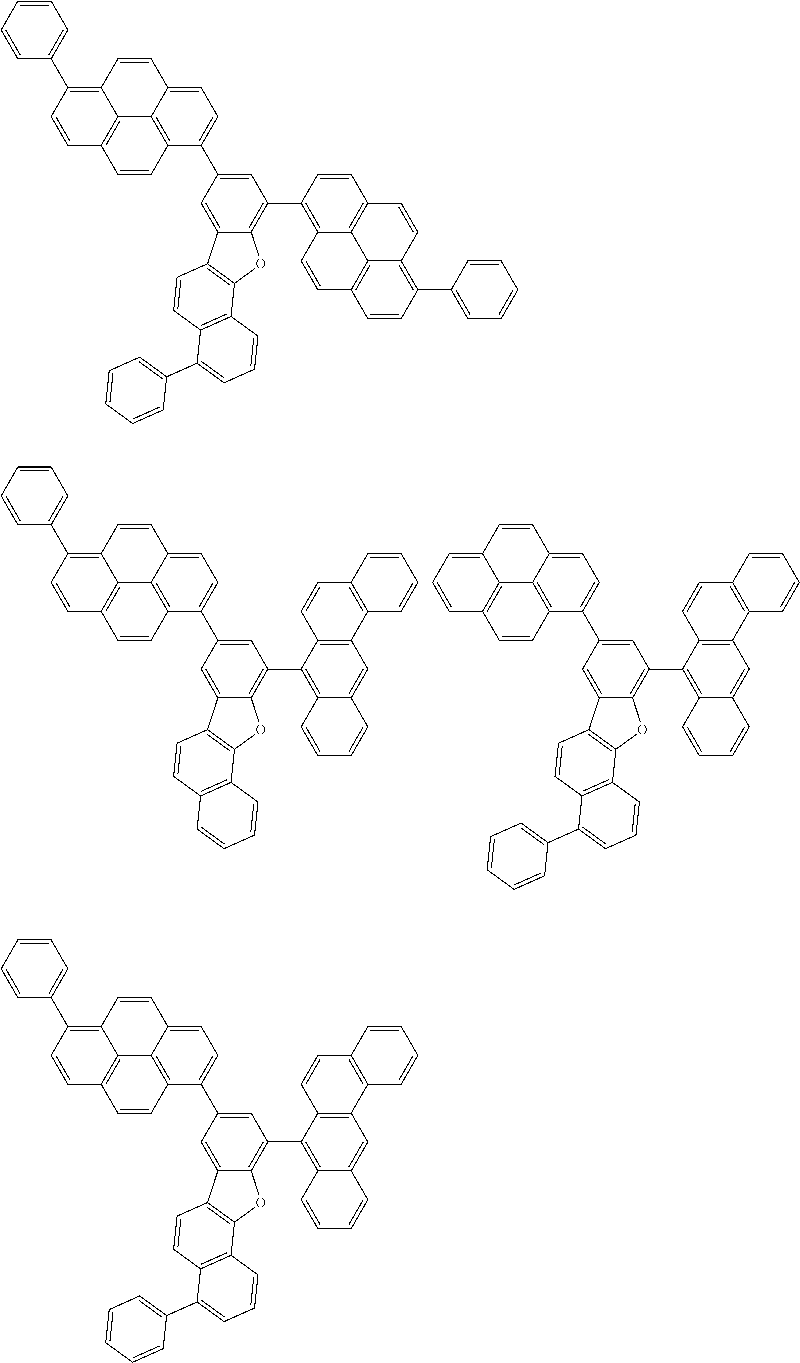

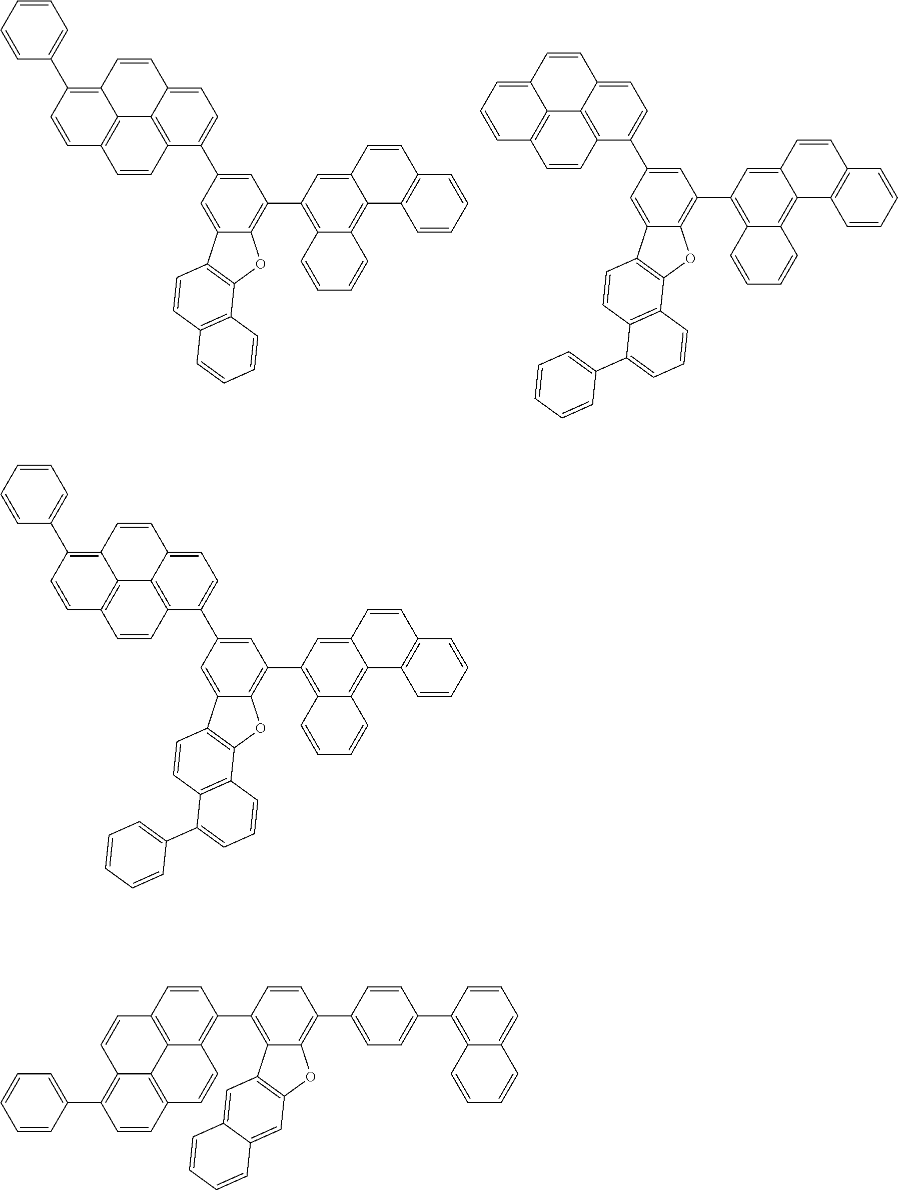 [SMILES: c1ccc(-c2ccc3ccc4c(-c5cc(-c6cc7ccc8ccccc8c7c7ccccc67)c6oc7c8ccccc8ccc7c6c5)ccc5ccc2c3c54)cc1 ; c1ccc(-c2cccc3c2ccc2c4cc(-c5ccc6ccc7cccc8ccc5c6c78)cc(-c5cc6ccc7ccccc7c6c6ccccc56)c4oc32)cc1 ; c1ccc(-c2cccc3c2ccc2c4cc(-c5ccc6ccc7c(-c8ccccc8)ccc8ccc5c6c87)cc(-c5cc6ccc7ccccc7c6c6ccccc56)c4oc32)cc1 ; c1ccc(-c2ccc3ccc4c(-c5ccc(-c6ccc(-c7cccc8ccccc78)cc6)c6oc7cc8ccccc8cc7c56)ccc5ccc2c3c54)cc1]

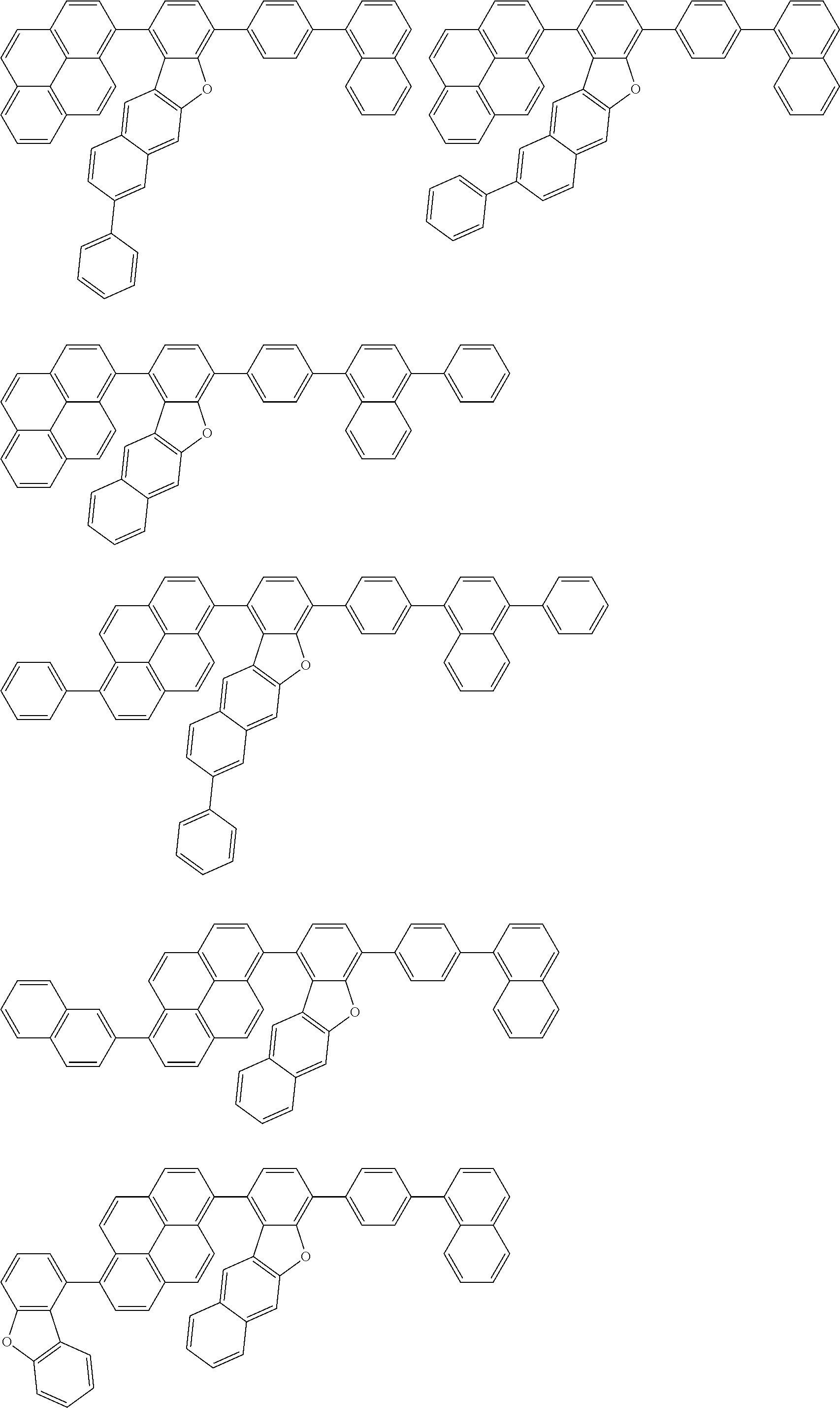

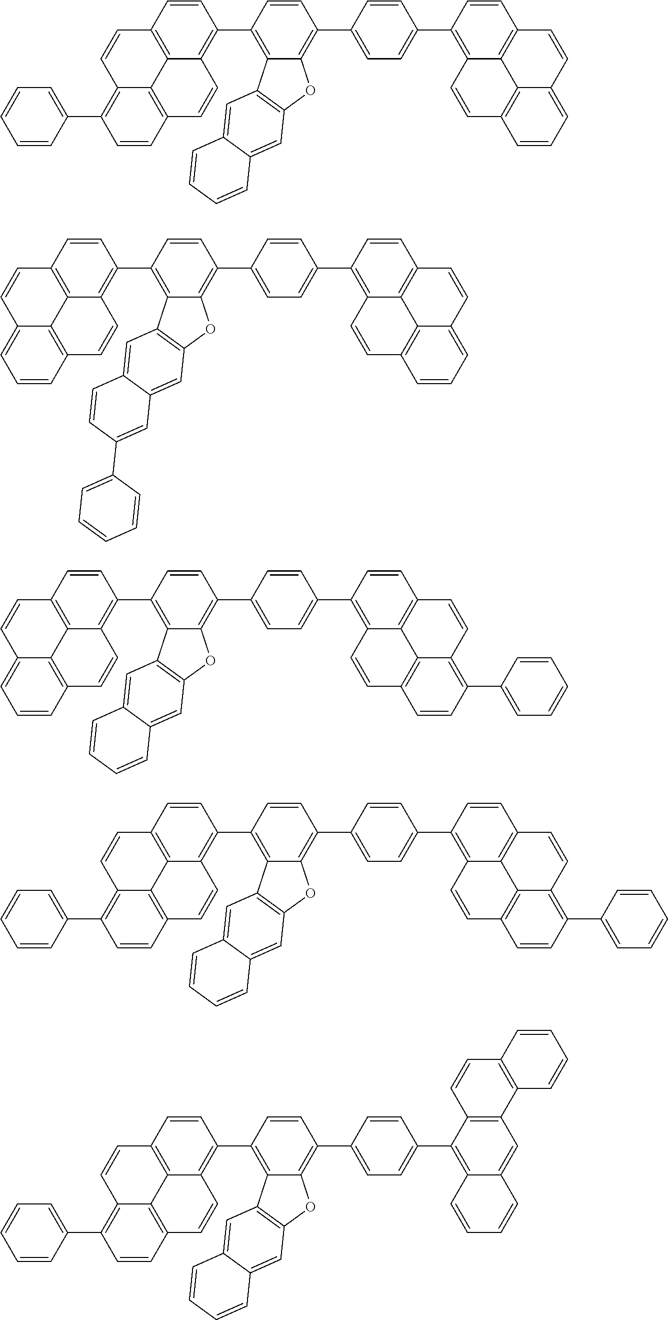

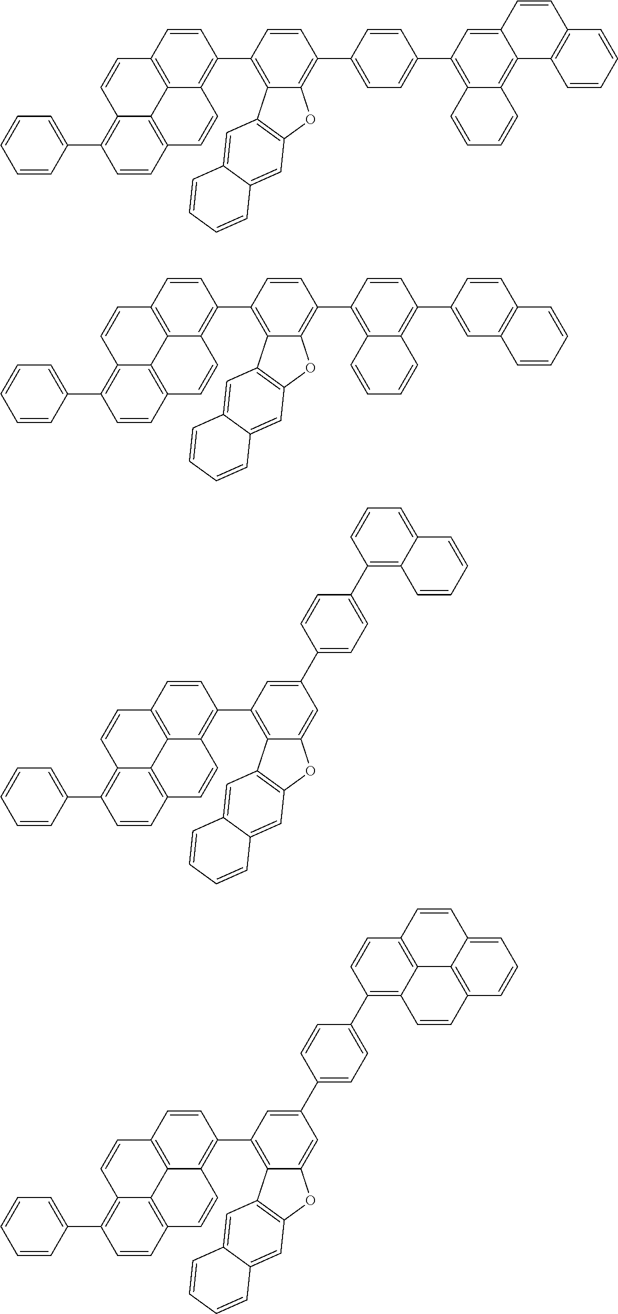 [SMILES: c1ccc(-c2ccc3ccc4c(-c5ccc(-c6ccc(-c7cc8ccc9ccccc9c8c8ccccc78)cc6)c6oc7cc8ccccc8cc7c56)ccc5ccc2c3c54)cc1 ; c1ccc(-c2ccc3ccc4c(-c5ccc(-c6ccc(-c7ccc8ccccc8c7)c7ccccc67)c6oc7cc8ccccc8cc7c56)ccc5ccc2c3c54)cc1 ; c1ccc(-c2ccc3ccc4c(-c5cc(-c6ccc(-c7cccc8ccccc78)cc6)cc6oc7cc8ccccc8cc7c56)ccc5ccc2c3c54)cc1 ; c1ccc(-c2ccc3ccc4c(-c5cc(-c6ccc(-c7ccc8ccc9cccc%10ccc7c8c9%10)cc6)cc6oc7cc8ccccc8cc7c56)ccc5ccc2c3c54)cc1]

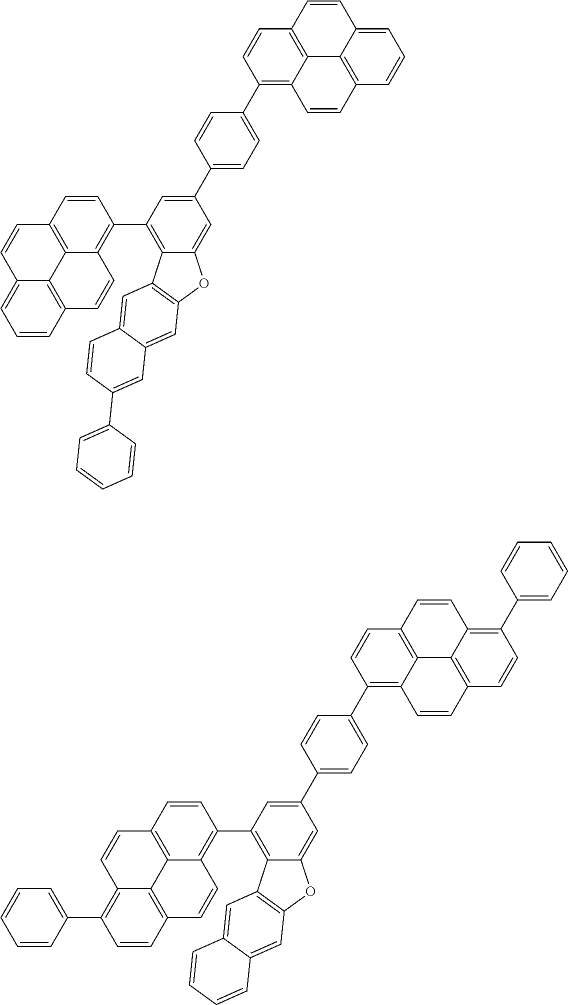 [SMILES: c1ccc(-c2ccc3cc4c(cc3c2)oc2cc(-c3ccc(-c5ccc6ccc7cccc8ccc5c6c78)cc3)cc(-c3ccc5ccc6cccc7ccc3c5c67)c24)cc1 ; c1ccc(-c2ccc3ccc4c(-c5ccc(-c6cc(-c7ccc8ccc9c(-c%10ccccc%10)ccc%10ccc7c8c%109)c7c(c6)oc6cc8ccccc8cc67)cc5)ccc5ccc2c3c54)cc1]

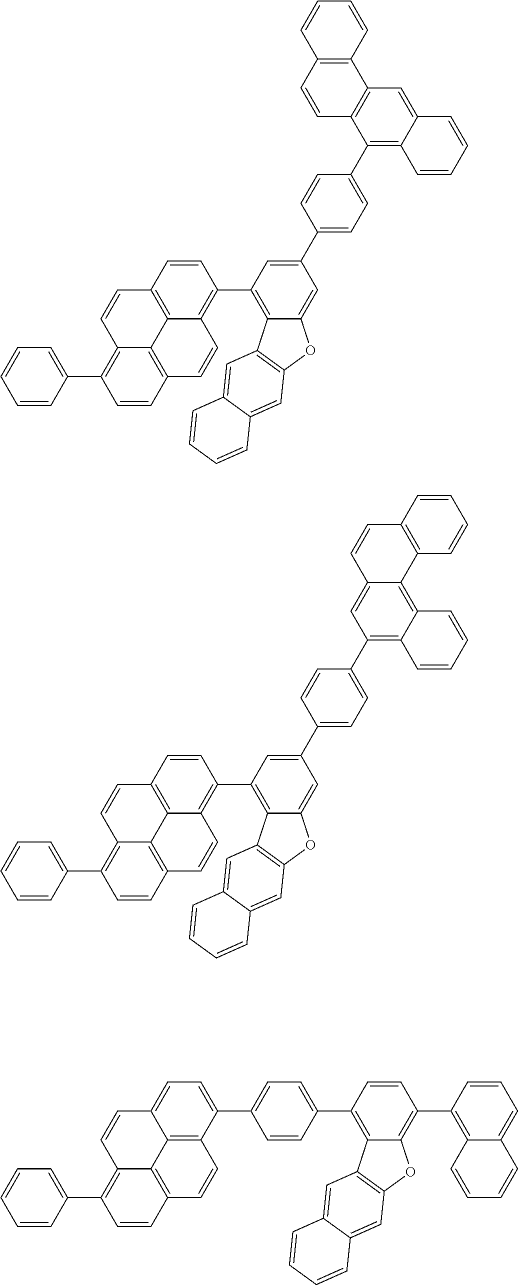 [SMILES: c1ccc(-c2ccc3ccc4c(-c5cc(-c6ccc(-c7c8ccccc8cc8c7ccc7ccccc78)cc6)cc6oc7cc8ccccc8cc7c56)ccc5ccc2c3c54)cc1 ; c1ccc(-c2ccc3ccc4c(-c5cc(-c6ccc(-c7cc8ccc9ccccc9c8c8ccccc78)cc6)cc6oc7cc8ccccc8cc7c56)ccc5ccc2c3c54)cc1 ; c1ccc(-c2ccc3ccc4c(-c5ccc(-c6ccc(-c7cccc8ccccc78)c7oc8cc9ccccc9cc8c67)cc5)ccc5ccc2c3c54)cc1]

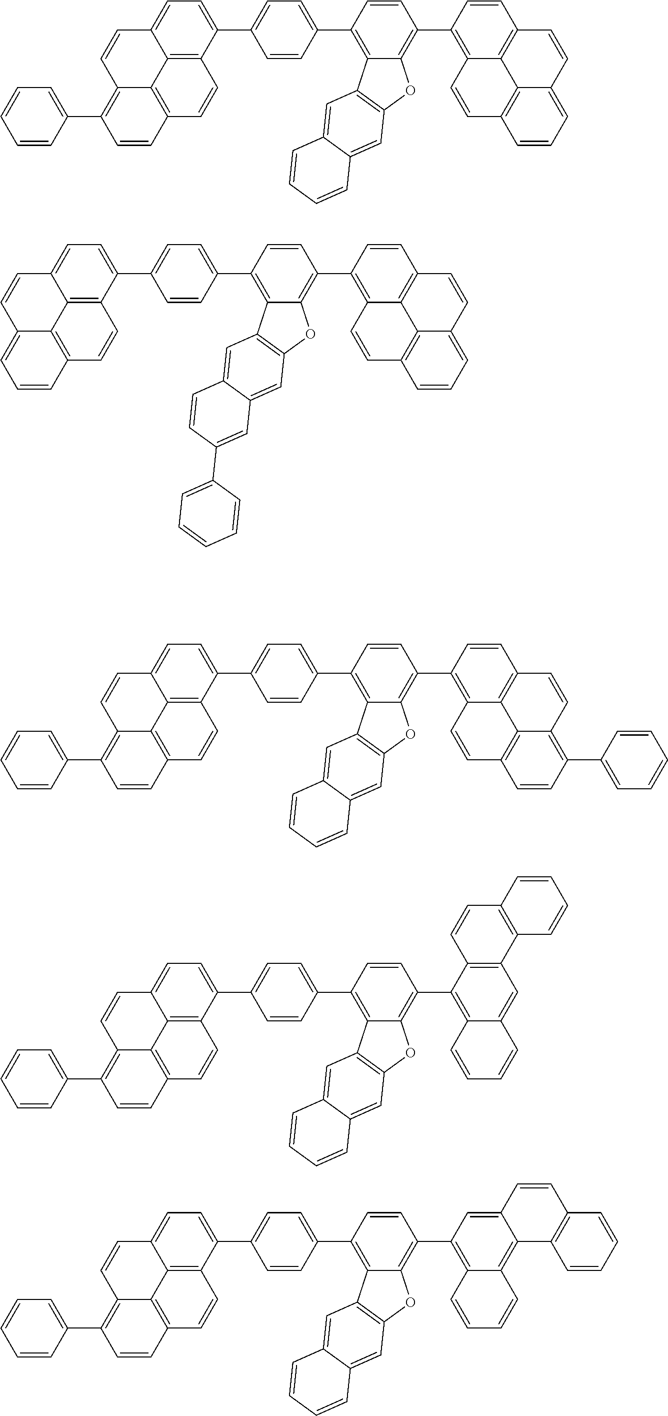

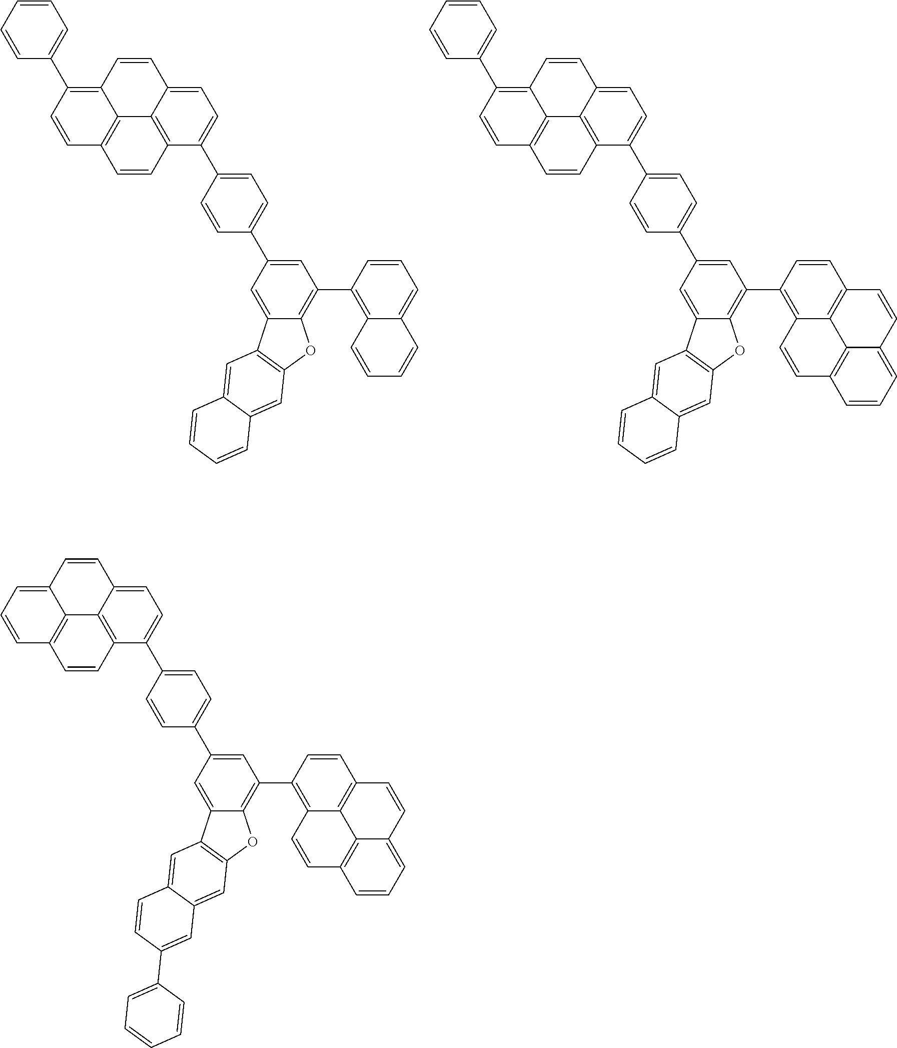 [SMILES: c1ccc(-c2ccc3ccc4c(-c5ccc(-c6cc(-c7cccc8ccccc78)c7oc8cc9ccccc9cc8c7c6)cc5)ccc5ccc2c3c54)cc1 ; c1ccc(-c2ccc3ccc4c(-c5ccc(-c6cc(-c7ccc8ccc9cccc%10ccc7c8c9%10)c7oc8cc9ccccc9cc8c7c6)cc5)ccc5ccc2c3c54)cc1 ; c1ccc(-c2ccc3cc4c(cc3c2)oc2c(-c3ccc5ccc6cccc7ccc3c5c67)cc(-c3ccc(-c5ccc6ccc7cccc8ccc5c6c78)cc3)cc24)cc1]

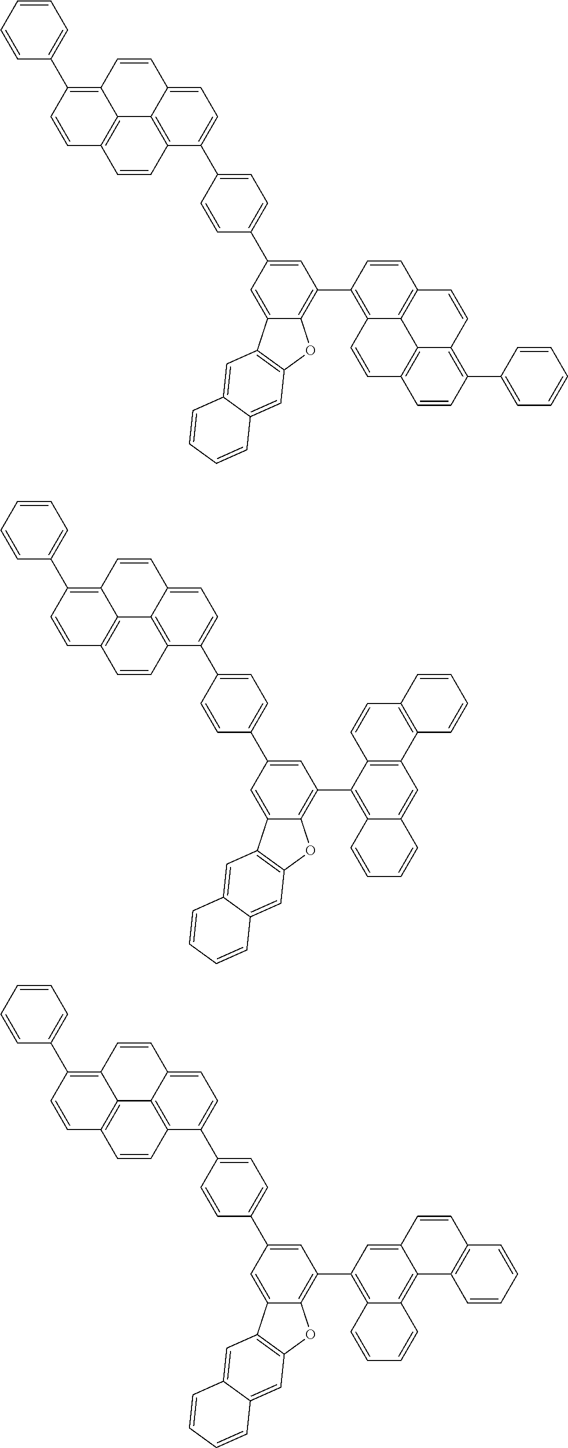 [SMILES: c1ccc(-c2ccc3ccc4c(-c5ccc(-c6cc(-c7ccc8ccc9c(-c%10ccccc%10)ccc%10ccc7c8c%109)c7oc8cc9ccccc9cc8c7c6)cc5)ccc5ccc2c3c54)cc1 ; c1ccc(-c2ccc3ccc4c(-c5ccc(-c6cc(-c7c8ccccc8cc8c7ccc7ccccc78)c7oc8cc9ccccc9cc8c7c6)cc5)ccc5ccc2c3c54)cc1 ; c1ccc(-c2ccc3ccc4c(-c5ccc(-c6cc(-c7cc8ccc9ccccc9c8c8ccccc78)c7oc8cc9ccccc9cc8c7c6)cc5)ccc5ccc2c3c54)cc1]

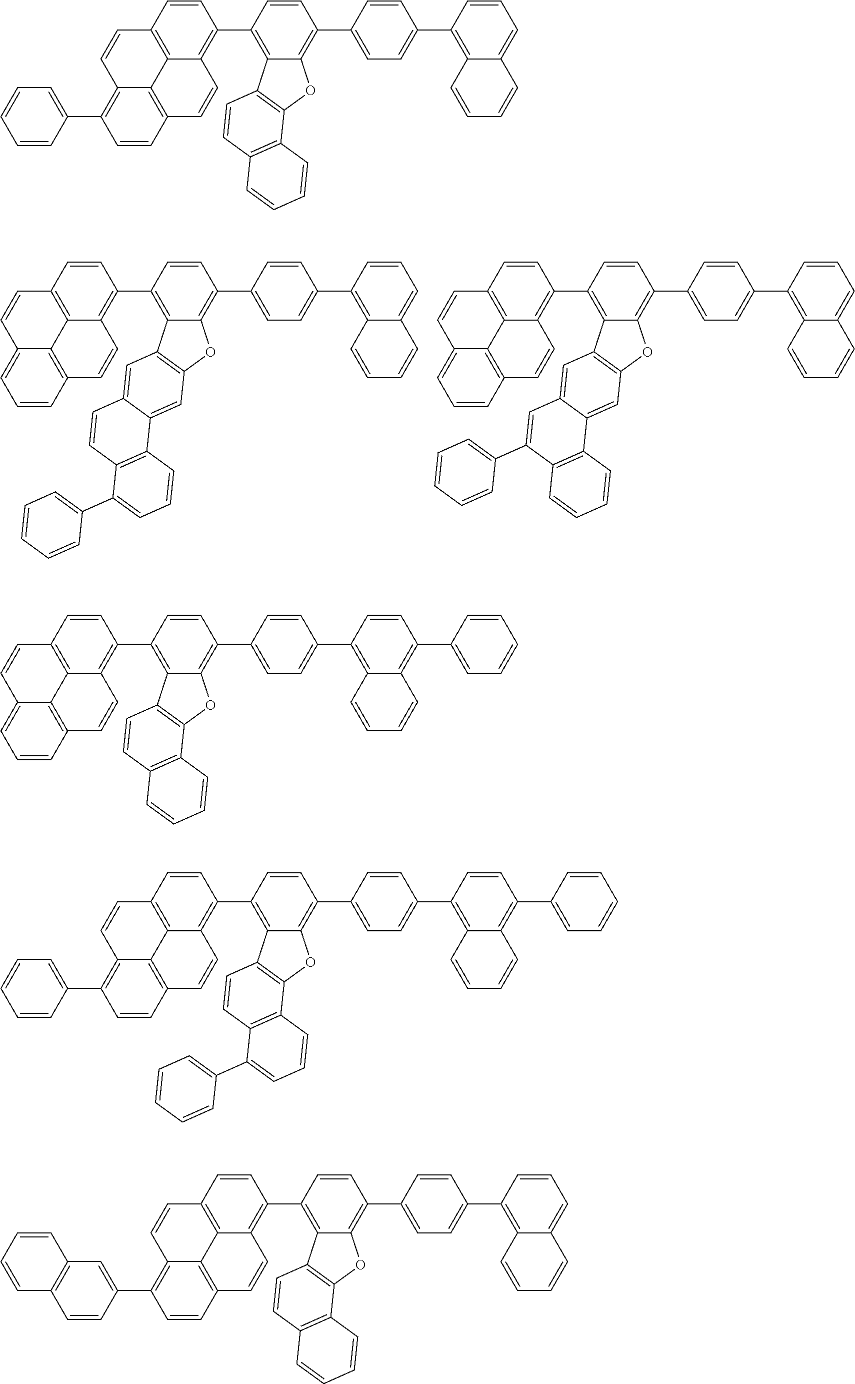

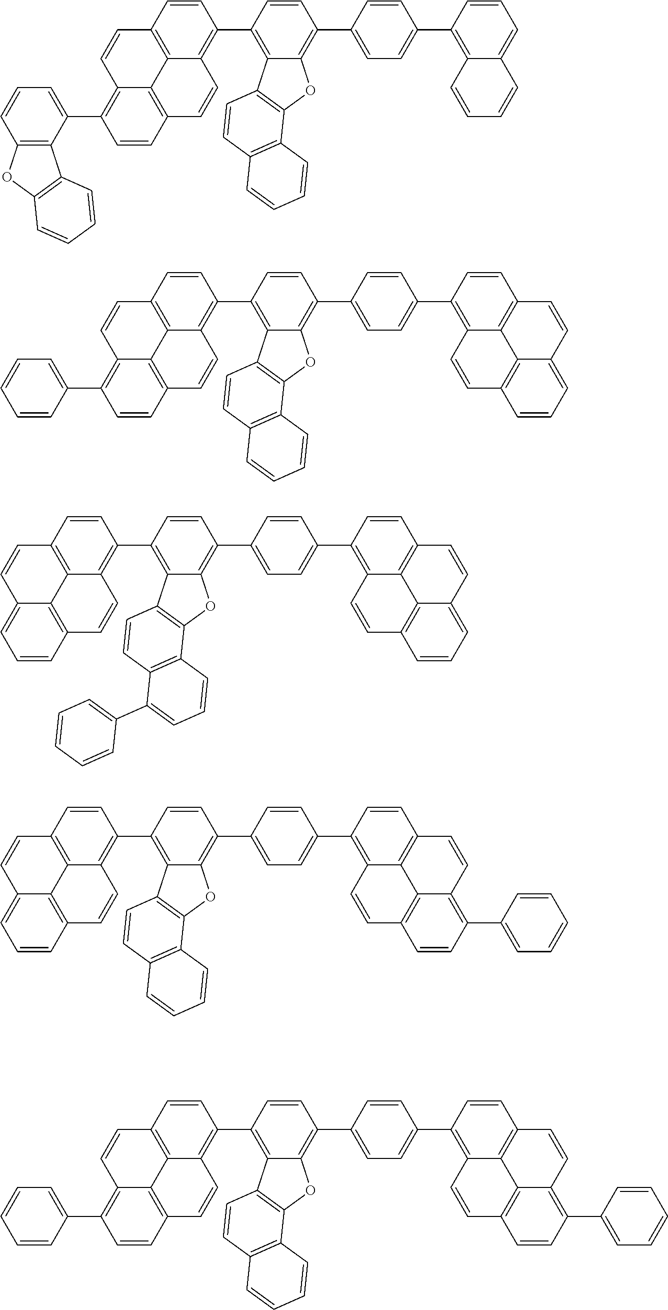

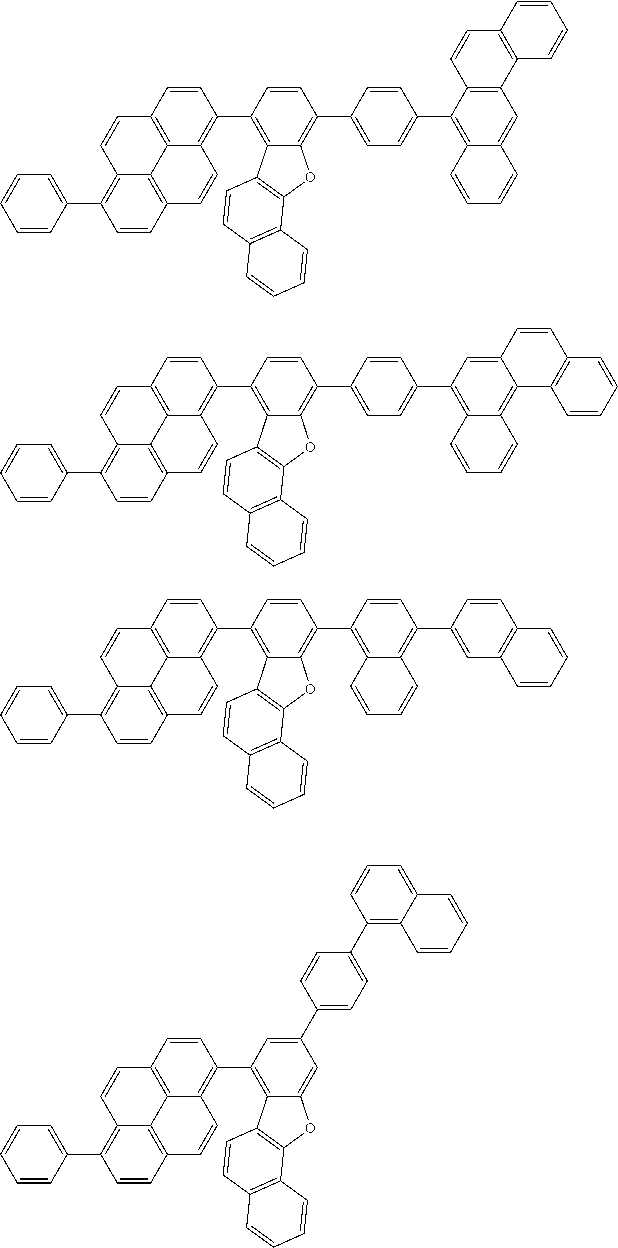

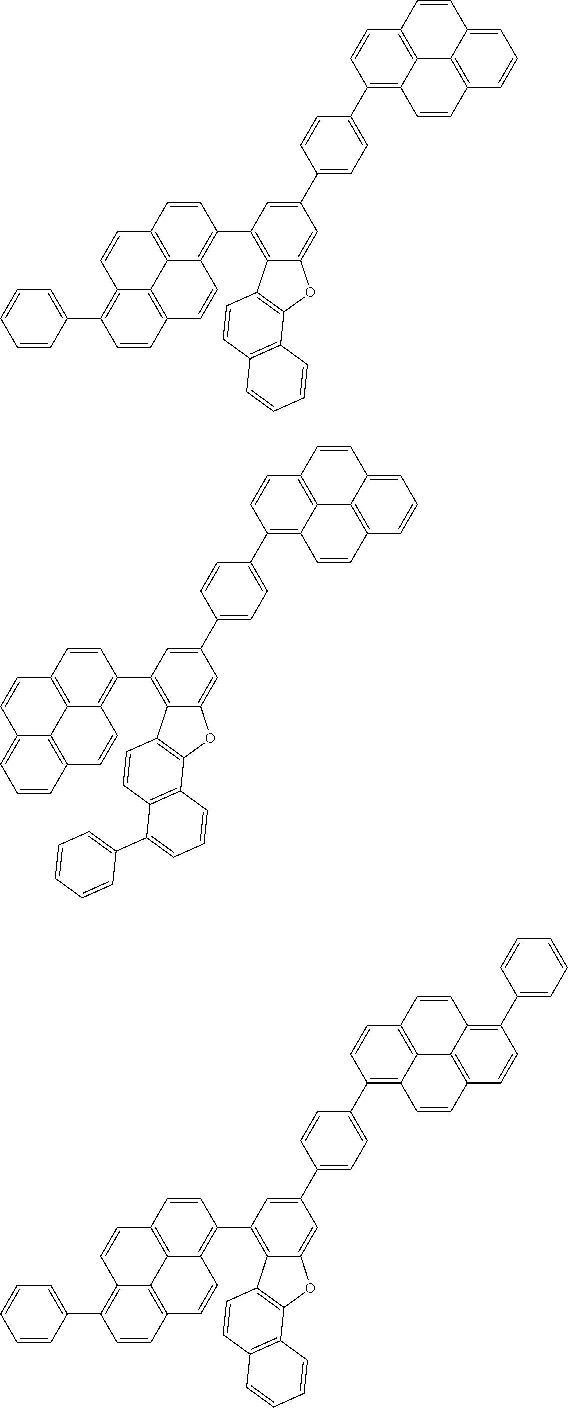 [SMILES: c1ccc(-c2ccc3ccc4c(-c5cc(-c6ccc(-c7ccc8ccc9cccc%10ccc7c8c9%10)cc6)cc6oc7c8ccccc8ccc7c56)ccc5ccc2c3c54)cc1 ; c1ccc(-c2cccc3c2ccc2c3oc3cc(-c4ccc(-c5ccc6ccc7cccc8ccc5c6c78)cc4)cc(-c4ccc5ccc6cccc7ccc4c5c67)c32)cc1 ; c1ccc(-c2ccc3ccc4c(-c5ccc(-c6cc(-c7ccc8ccc9c(-c%10ccccc%10)ccc%10ccc7c8c%109)c7c(c6)oc6c8ccccc8ccc67)cc5)ccc5ccc2c3c54)cc1]

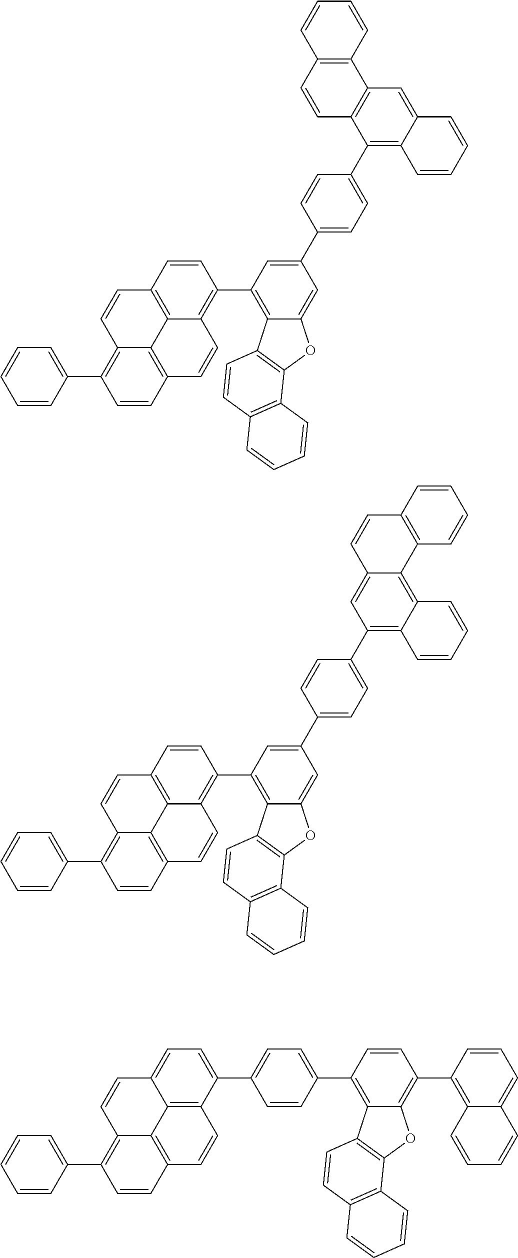 [SMILES: c1ccc(-c2ccc3ccc4c(-c5cc(-c6ccc(-c7c8ccccc8cc8c7ccc7ccccc78)cc6)cc6oc7c8ccccc8ccc7c56)ccc5ccc2c3c54)cc1 ; c1ccc(-c2ccc3ccc4c(-c5cc(-c6ccc(-c7cc8ccc9ccccc9c8c8ccccc78)cc6)cc6oc7c8ccccc8ccc7c56)ccc5ccc2c3c54)cc1 ; c1ccc(-c2ccc3ccc4c(-c5ccc(-c6ccc(-c7cccc8ccccc78)c7oc8c9ccccc9ccc8c67)cc5)ccc5ccc2c3c54)cc1]

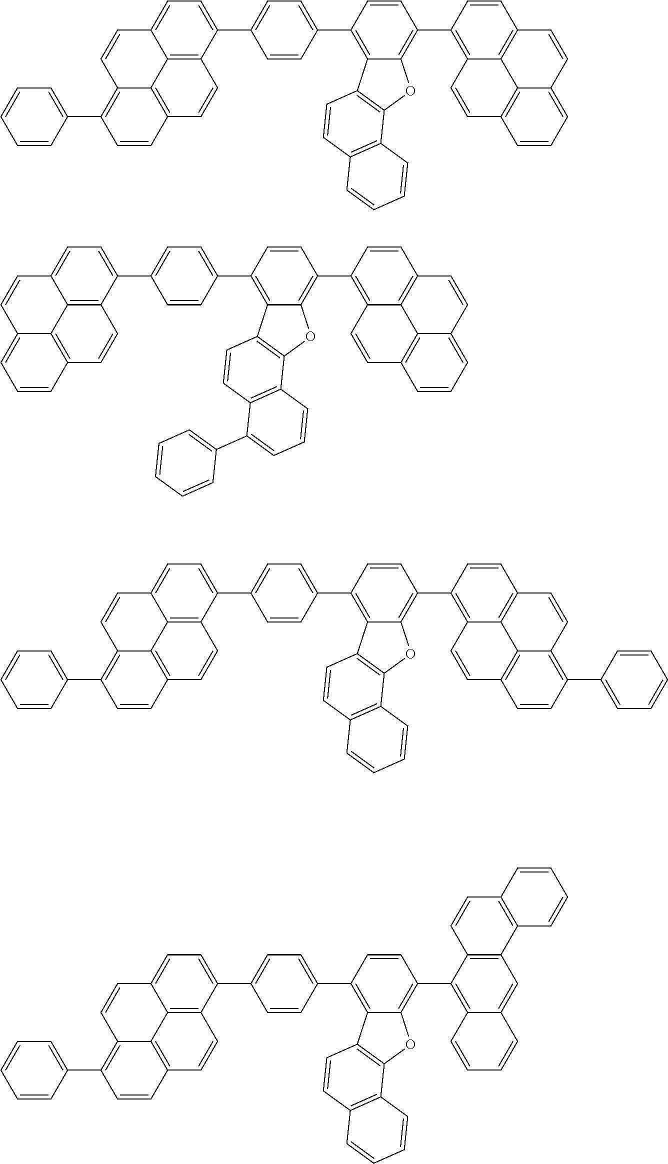

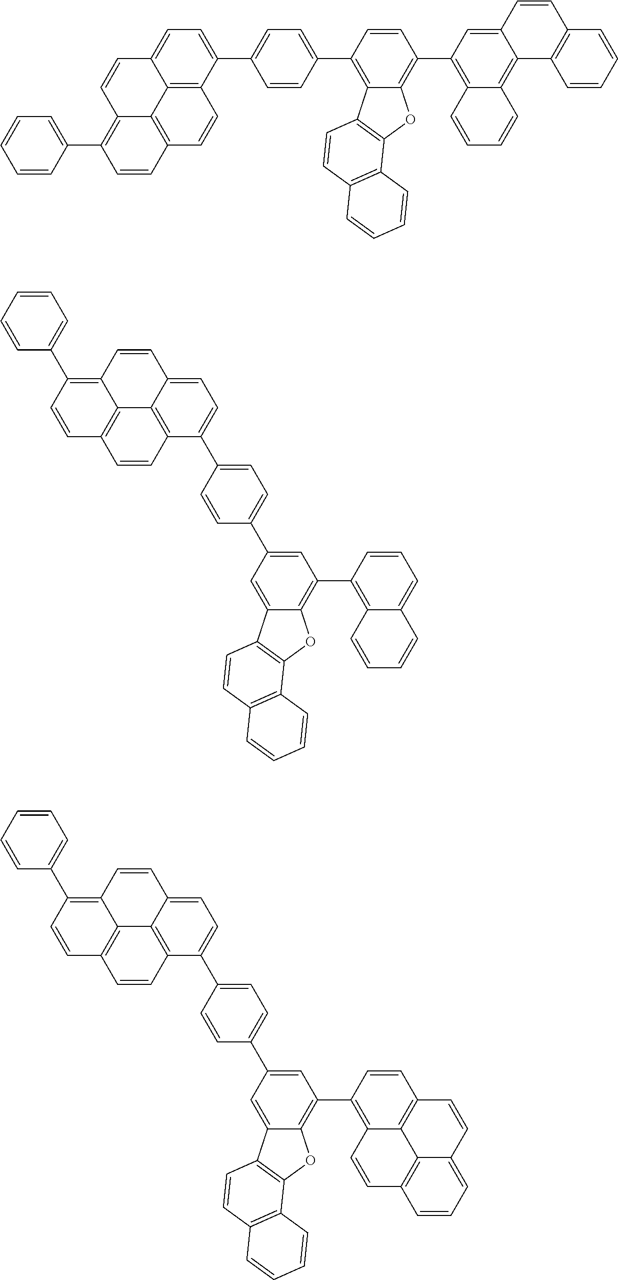 [SMILES: c1ccc(-c2ccc3ccc4c(-c5ccc(-c6ccc(-c7cc8ccc9ccccc9c8c8ccccc78)c7oc8c9ccccc9ccc8c67)cc5)ccc5ccc2c3c54)cc1 ; c1ccc(-c2ccc3ccc4c(-c5ccc(-c6cc(-c7cccc8ccccc78)c7oc8c9ccccc9ccc8c7c6)cc5)ccc5ccc2c3c54)cc1 ; c1ccc(-c2ccc3ccc4c(-c5ccc(-c6cc(-c7ccc8ccc9cccc%10ccc7c8c9%10)c7oc8c9ccccc9ccc8c7c6)cc5)ccc5ccc2c3c54)cc1]

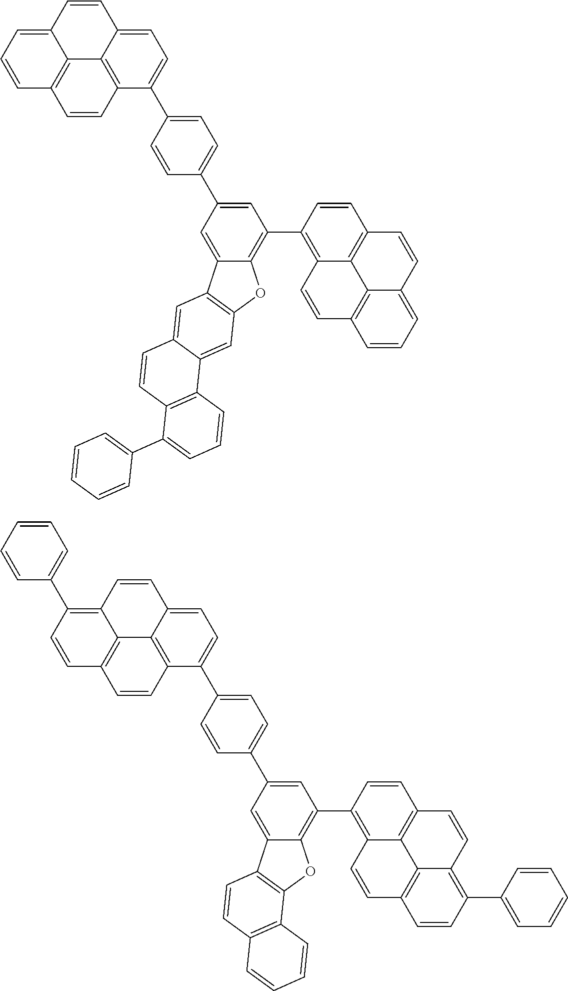 [SMILES: c1ccc(-c2cccc3c2ccc2cc4c(cc23)oc2c(-c3ccc5ccc6cccc7ccc3c5c67)cc(-c3ccc(-c5ccc6ccc7cccc8ccc5c6c78)cc3)cc24)cc1 ; c1ccc(-c2ccc3ccc4c(-c5ccc(-c6cc(-c7ccc8ccc9c(-c%10ccccc%10)ccc%10ccc7c8c%109)c7oc8c9ccccc9ccc8c7c6)cc5)ccc5ccc2c3c54)cc1]

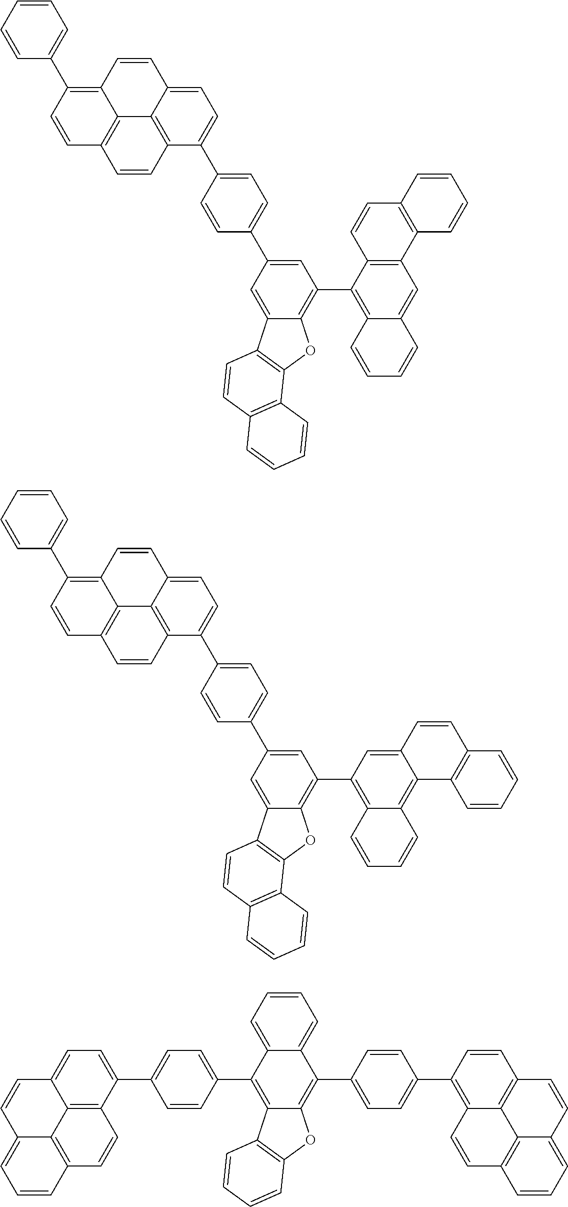

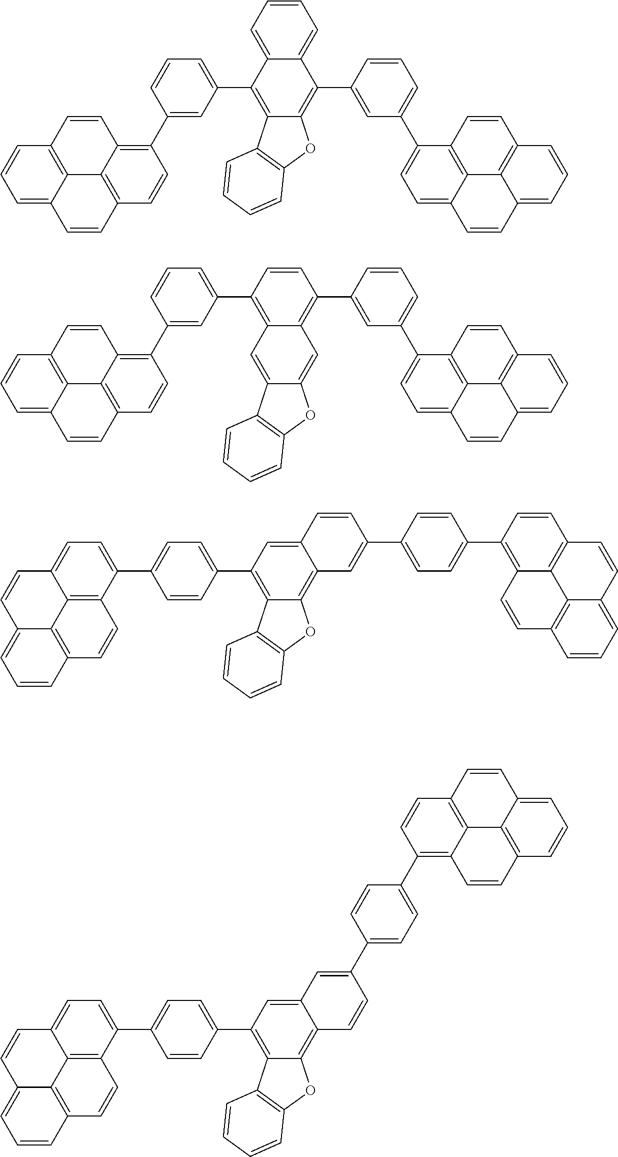 [SMILES: c1cc(-c2ccc3ccc4cccc5ccc2c3c45)cc(-c2c3ccccc3c(-c3cccc(-c4ccc5ccc6cccc7ccc4c5c67)c3)c3c2oc2ccccc23)c1 ; c1cc(-c2ccc(-c3cccc(-c4ccc5ccc6cccc7ccc4c5c67)c3)c3cc4c(cc23)oc2ccccc24)cc(-c2ccc3ccc4cccc5ccc2c3c45)c1 ; c1cc2ccc3ccc(-c4ccc(-c5ccc6cc(-c7ccc(-c8ccc9ccc%10cccc%11ccc8c9c%10%11)cc7)c7c8ccccc8oc7c6c5)cc4)c4ccc(c1)c2c34 ; c1cc2ccc3ccc(-c4ccc(-c5ccc6c(c5)cc(-c5ccc(-c7ccc8ccc9cccc%10ccc7c8c9%10)cc5)c5c7ccccc7oc65)cc4)c4ccc(c1)c2c34]

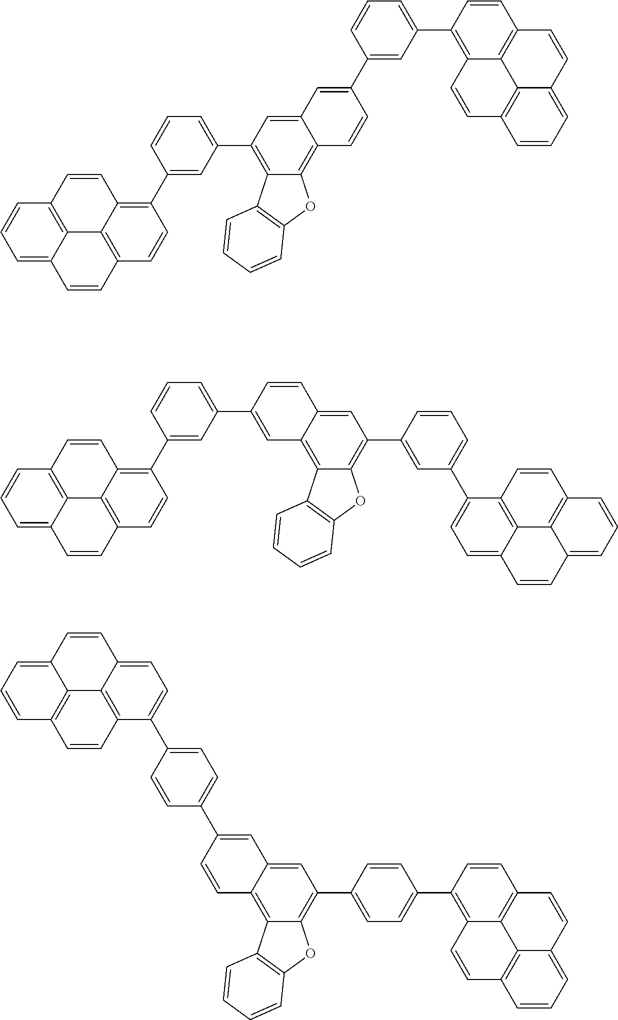 [SMILES: c1cc(-c2ccc3c(c2)cc(-c2cccc(-c4ccc5ccc6cccc7ccc4c5c67)c2)c2c4ccccc4oc32)cc(-c2ccc3ccc4cccc5ccc2c3c45)c1 ; c1cc(-c2ccc3cc(-c4cccc(-c5ccc6ccc7cccc8ccc5c6c78)c4)c4oc5ccccc5c4c3c2)cc(-c2ccc3ccc4cccc5ccc2c3c45)c1 ; c1cc2ccc3ccc(-c4ccc(-c5ccc6c(c5)cc(-c5ccc(-c7ccc8ccc9cccc%10ccc7c8c9%10)cc5)c5oc7ccccc7c56)cc4)c4ccc(c1)c2c34]

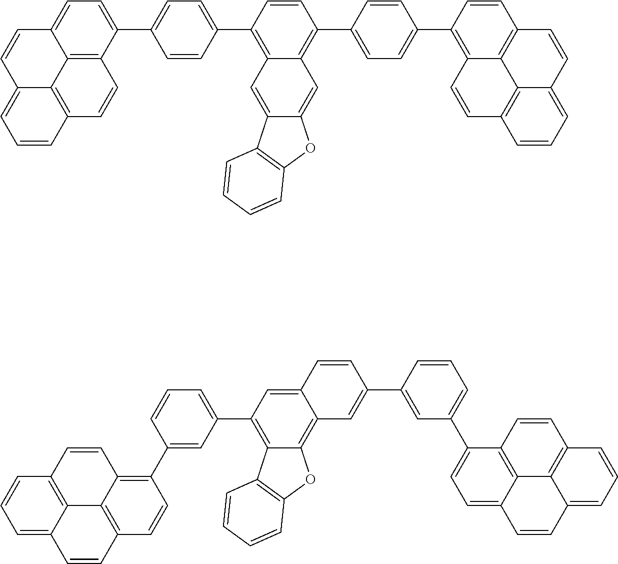 [SMILES: c1cc2ccc3ccc(-c4ccc(-c5ccc(-c6ccc(-c7ccc8ccc9cccc%10ccc7c8c9%10)cc6)c6cc7c(cc56)oc5ccccc57)cc4)c4ccc(c1)c2c34 ; c1cc(-c2ccc3cc(-c4cccc(-c5ccc6ccc7cccc8ccc5c6c78)c4)c4c5ccccc5oc4c3c2)cc(-c2ccc3ccc4cccc5ccc2c3c45)c1]

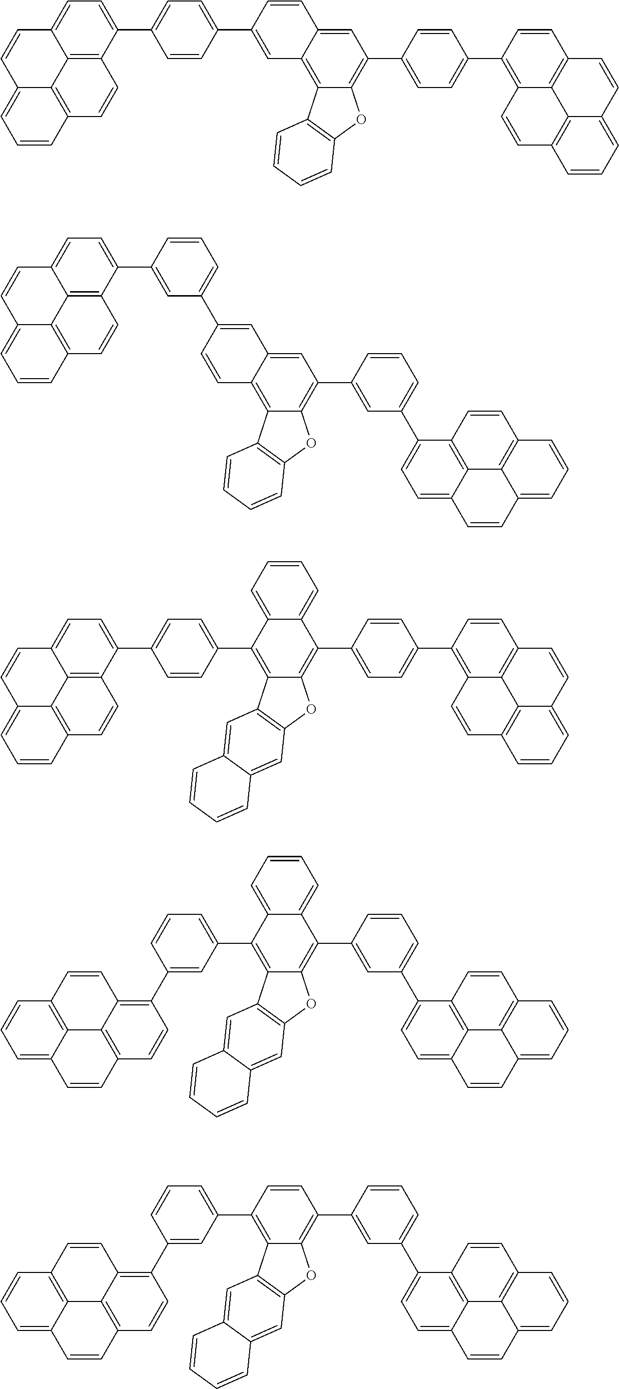

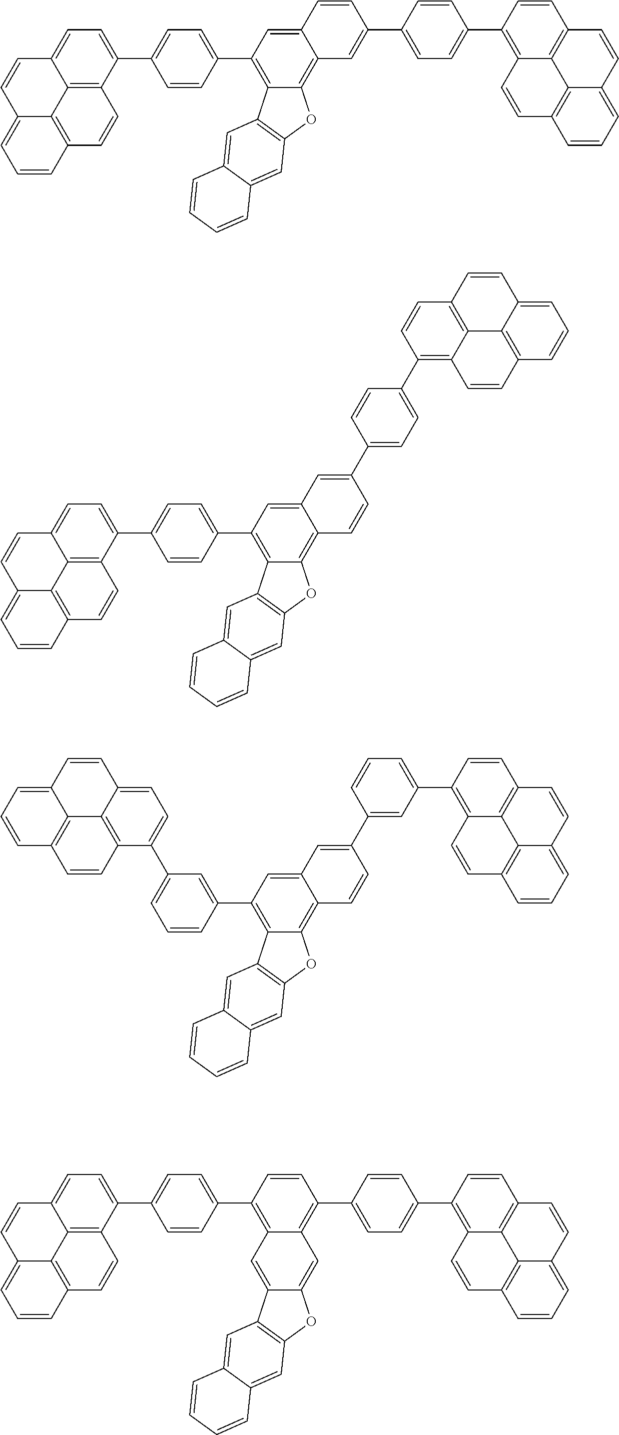 [SMILES: c1ccc2cc3c(cc2c1)oc1c2cc(-c4ccc(-c5ccc6ccc7cccc8ccc5c6c78)cc4)ccc2cc(-c2ccc(-c4ccc5ccc6cccc7ccc4c5c67)cc2)c31 ; c1ccc2cc3c(cc2c1)oc1c2ccc(-c4ccc(-c5ccc6ccc7cccc8ccc5c6c78)cc4)cc2cc(-c2ccc(-c4ccc5ccc6cccc7ccc4c5c67)cc2)c31 ; c1cc(-c2ccc3c(c2)cc(-c2cccc(-c4ccc5ccc6cccc7ccc4c5c67)c2)c2c4cc5ccccc5cc4oc32)cc(-c2ccc3ccc4cccc5ccc2c3c45)c1 ; c1ccc2cc3c(cc2c1)oc1cc2c(-c4ccc(-c5ccc6ccc7cccc8ccc5c6c78)cc4)ccc(-c4ccc(-c5ccc6ccc7cccc8ccc5c6c78)cc4)c2cc13]

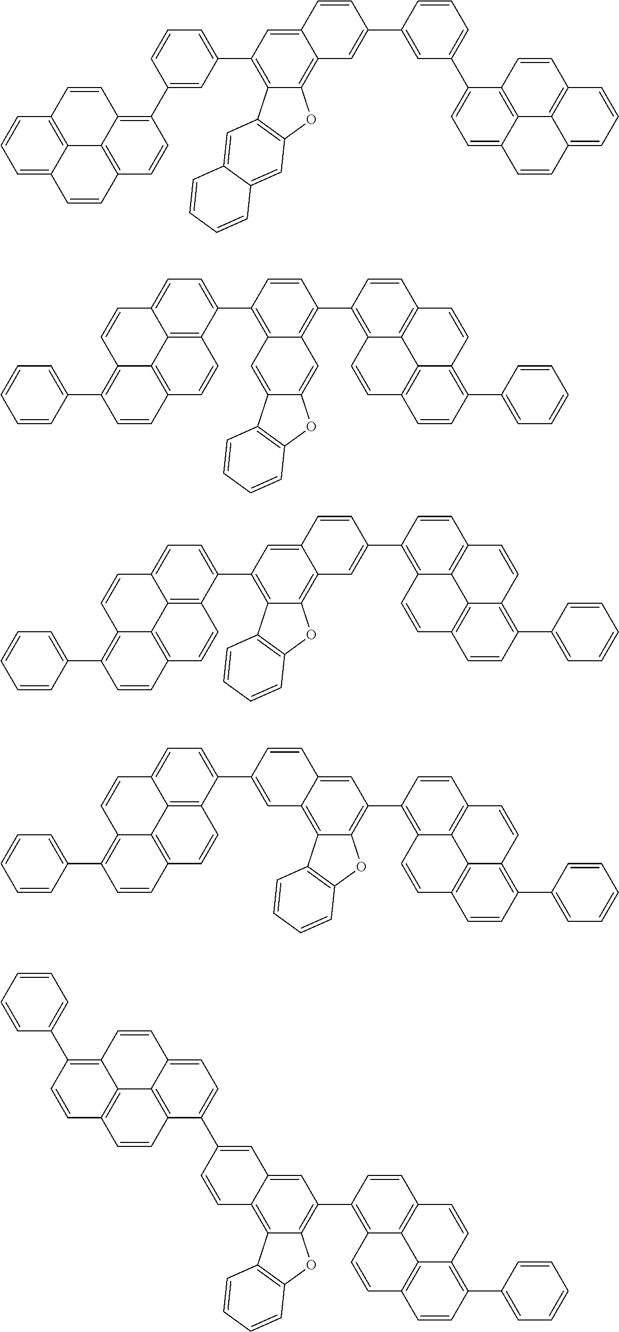

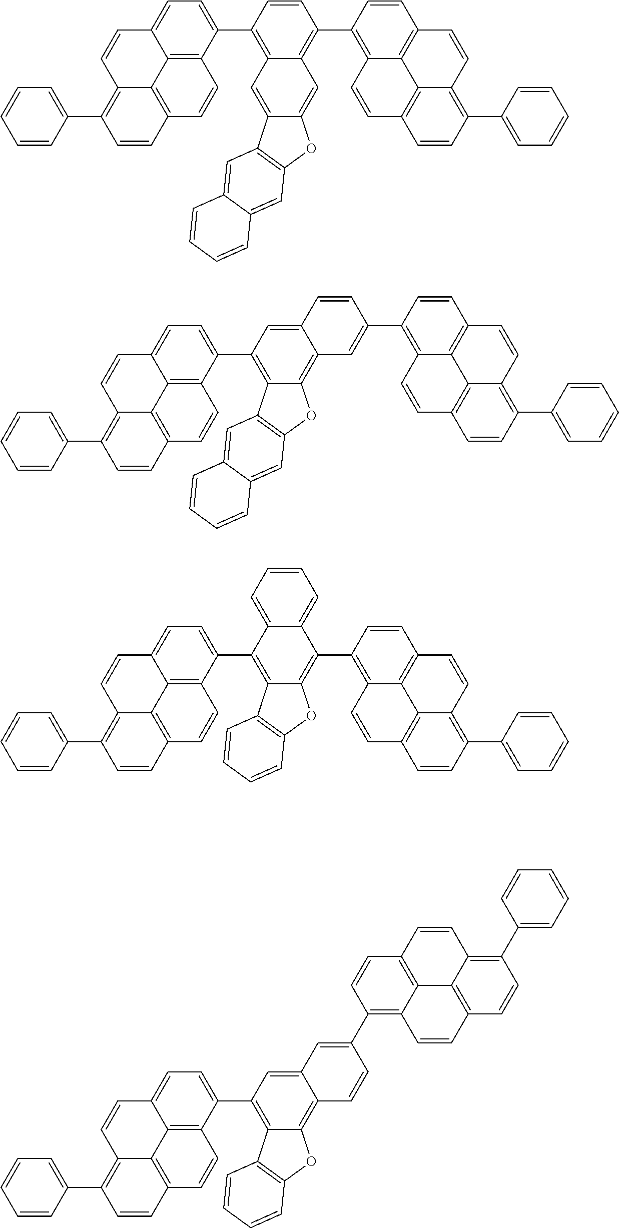 [SMILES: c1ccc(-c2ccc3ccc4c(-c5ccc(-c6ccc7ccc8c(-c9ccccc9)ccc9ccc6c7c98)c6cc7c(cc56)oc5cc6ccccc6cc57)ccc5ccc2c3c54)cc1 ; c1ccc(-c2ccc3ccc4c(-c5ccc6cc(-c7ccc8ccc9c(-c%10ccccc%10)ccc%10ccc7c8c%109)c7c8cc9ccccc9cc8oc7c6c5)ccc5ccc2c3c54)cc1 ; c1ccc(-c2ccc3ccc4c(-c5c6ccccc6c(-c6ccc7ccc8c(-c9ccccc9)ccc9ccc6c7c98)c6c5oc5ccccc56)ccc5ccc2c3c54)cc1 ; c1ccc(-c2ccc3ccc4c(-c5ccc6c(c5)cc(-c5ccc7ccc8c(-c9ccccc9)ccc9ccc5c7c98)c5c7ccccc7oc65)ccc5ccc2c3c54)cc1]

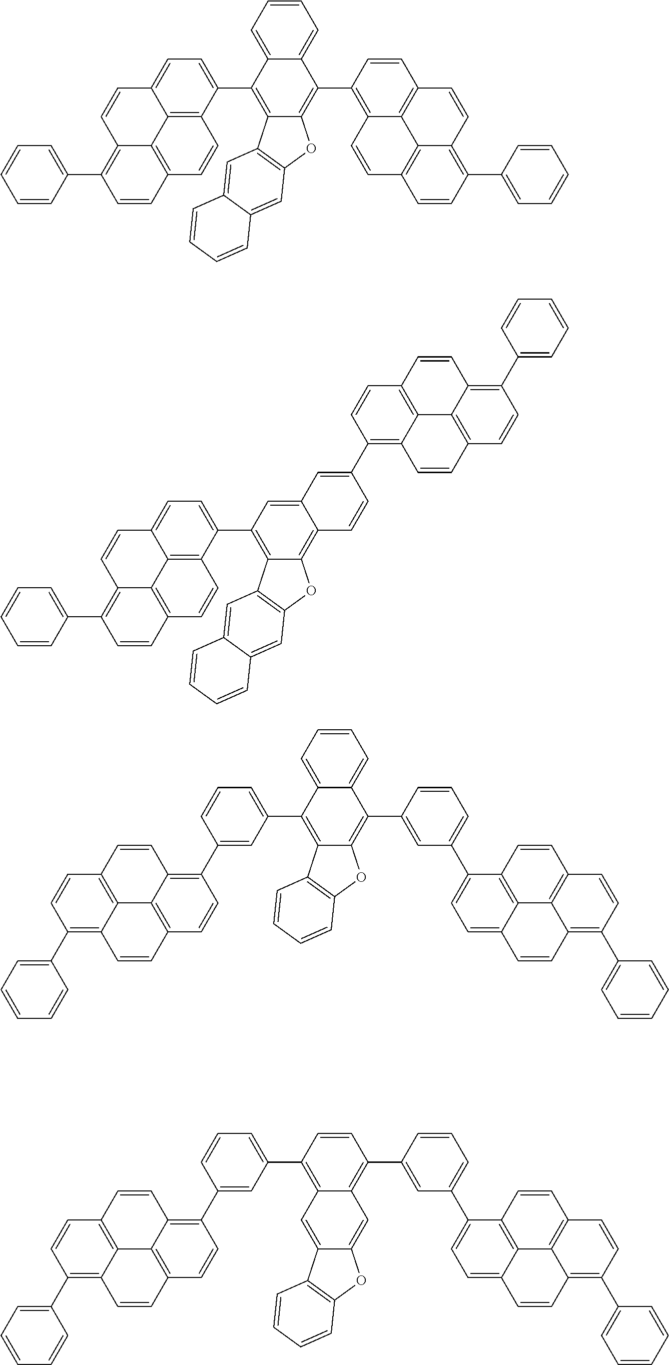 [SMILES: c1ccc(-c2ccc3ccc4c(-c5c6ccccc6c(-c6ccc7ccc8c(-c9ccccc9)ccc9ccc6c7c98)c6c5oc5cc7ccccc7cc56)ccc5ccc2c3c54)cc1 ; c1ccc(-c2ccc3ccc4c(-c5ccc6c(c5)cc(-c5ccc7ccc8c(-c9ccccc9)ccc9ccc5c7c98)c5c7cc8ccccc8cc7oc65)ccc5ccc2c3c54)cc1 ; c1ccc(-c2ccc3ccc4c(-c5cccc(-c6c7ccccc7c(-c7cccc(-c8ccc9ccc%10c(-c%11ccccc%11)ccc%11ccc8c9c%11%10)c7)c7c6oc6ccccc67)c5)ccc5ccc2c3c54)cc1 ; c1ccc(-c2ccc3ccc4c(-c5cccc(-c6ccc(-c7cccc(-c8ccc9ccc%10c(-c%11ccccc%11)ccc%11ccc8c9c%11%10)c7)c7cc8c(cc67)oc6ccccc68)c5)ccc5ccc2c3c54)cc1]

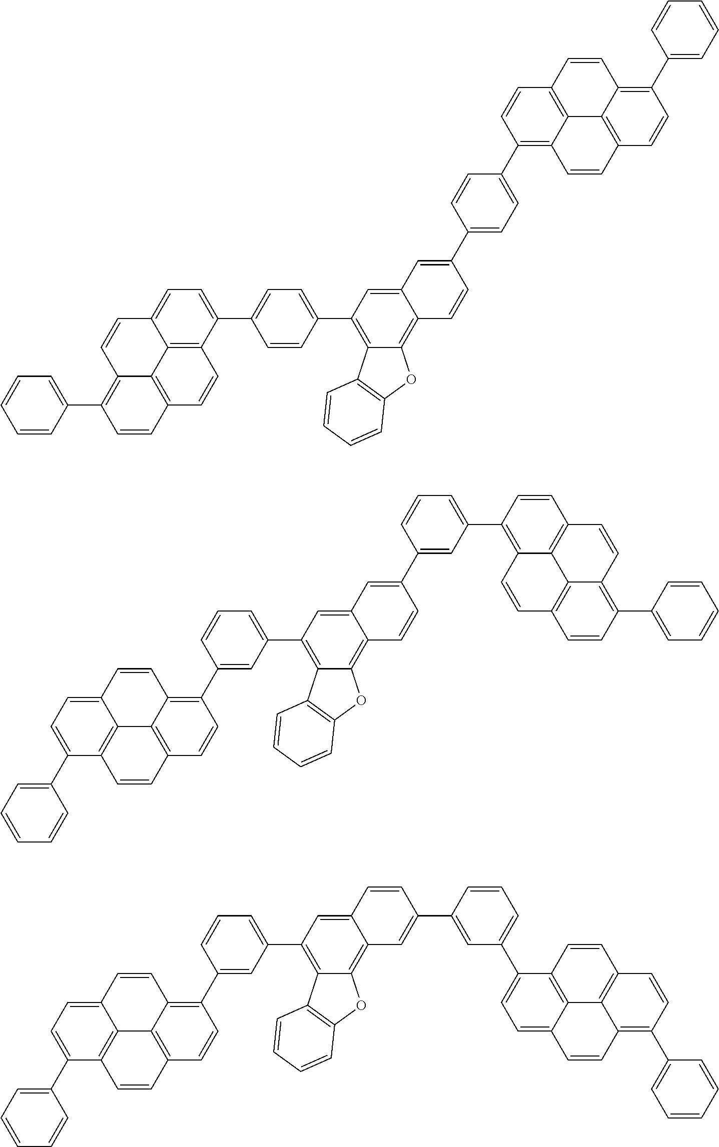 [SMILES: c1ccc(-c2ccc3ccc4c(-c5ccc(-c6ccc7c(c6)cc(-c6ccc(-c8ccc9ccc%10c(-c%11ccccc%11)ccc%11ccc8c9c%11%10)cc6)c6c8ccccc8oc76)cc5)ccc5ccc2c3c54)cc1 ; c1ccc(-c2ccc3ccc4c(-c5cccc(-c6ccc7c(c6)cc(-c6cccc(-c8ccc9ccc%10c(-c%11ccccc%11)ccc%11ccc8c9c%11%10)c6)c6c8ccccc8oc76)c5)ccc5ccc2c3c54)cc1 ; c1ccc(-c2ccc3ccc4c(-c5cccc(-c6ccc7cc(-c8cccc(-c9ccc%10ccc%11c(-c%12ccccc%12)ccc%12ccc9c%10c%12%11)c8)c8c9ccccc9oc8c7c6)c5)ccc5ccc2c3c54)cc1]

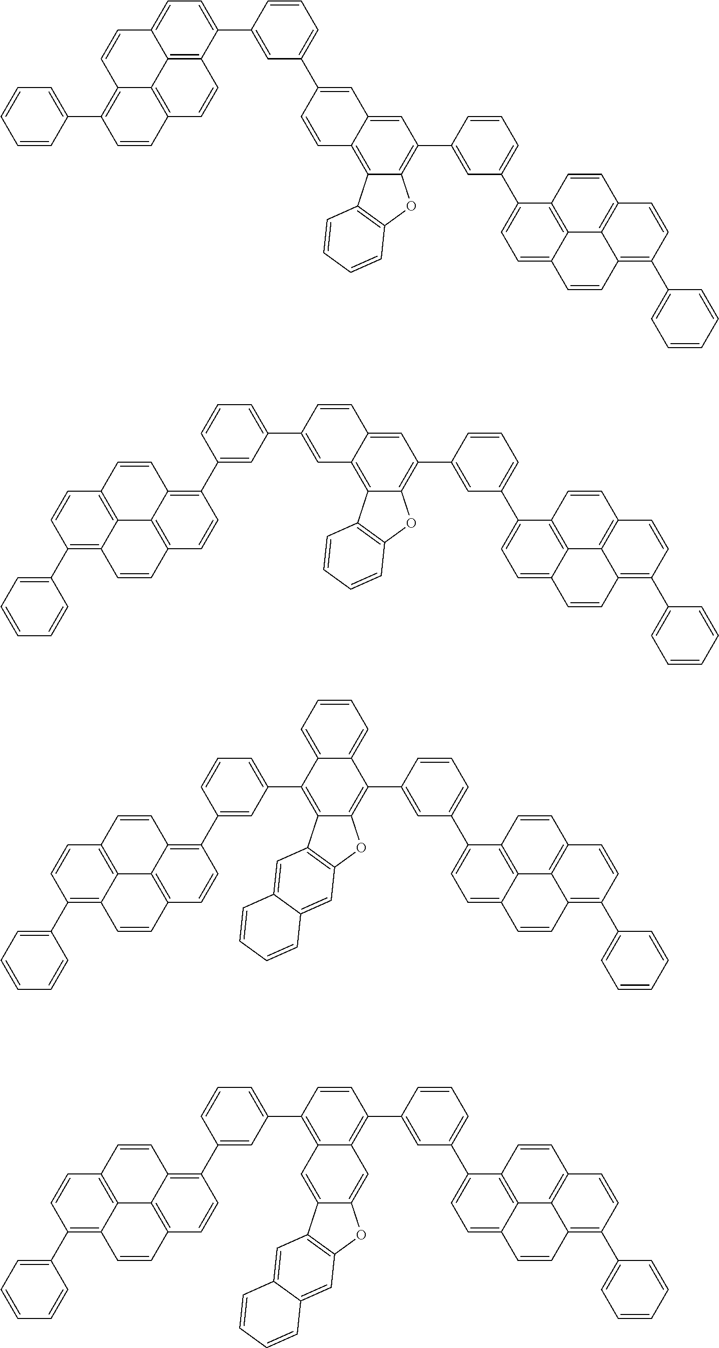

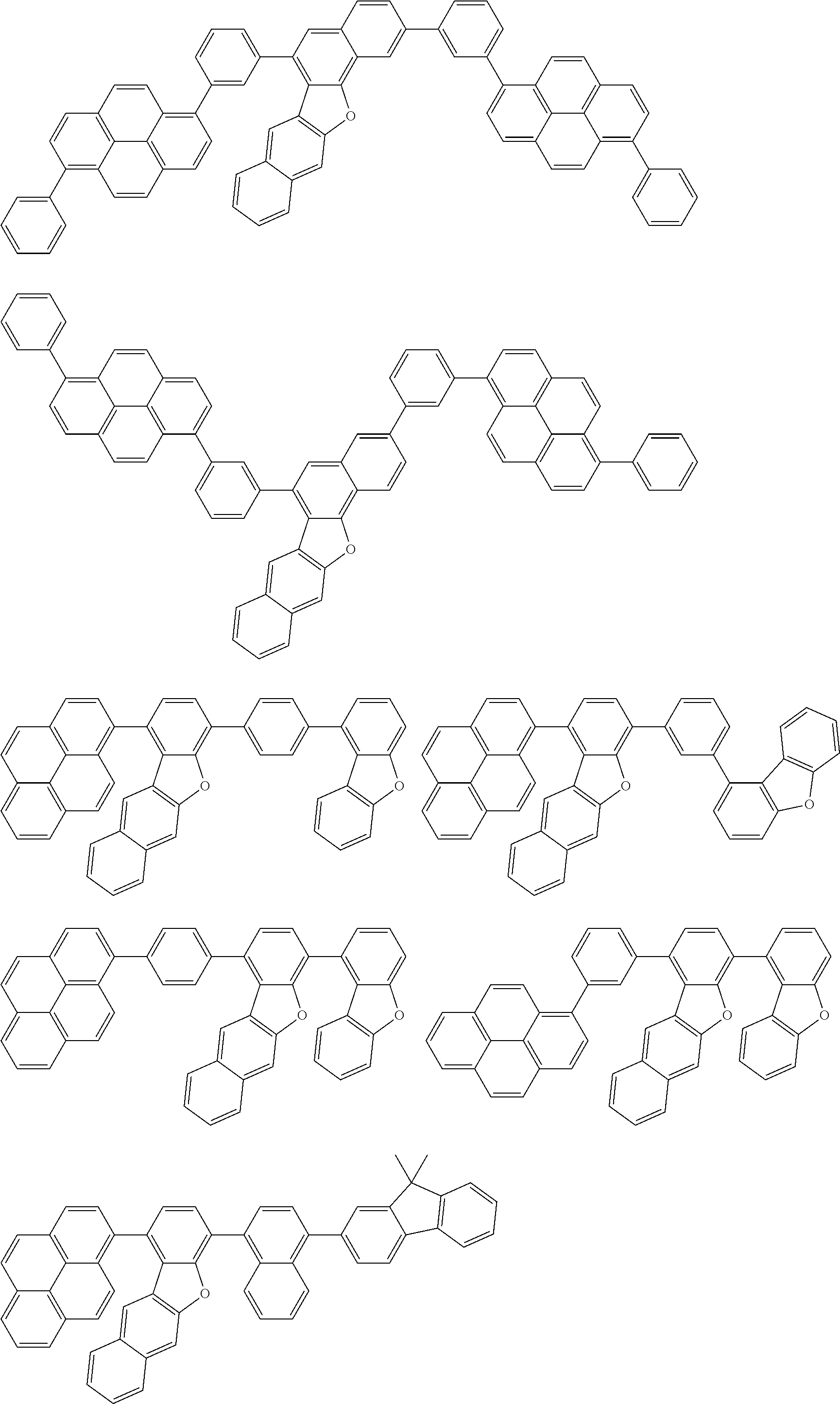

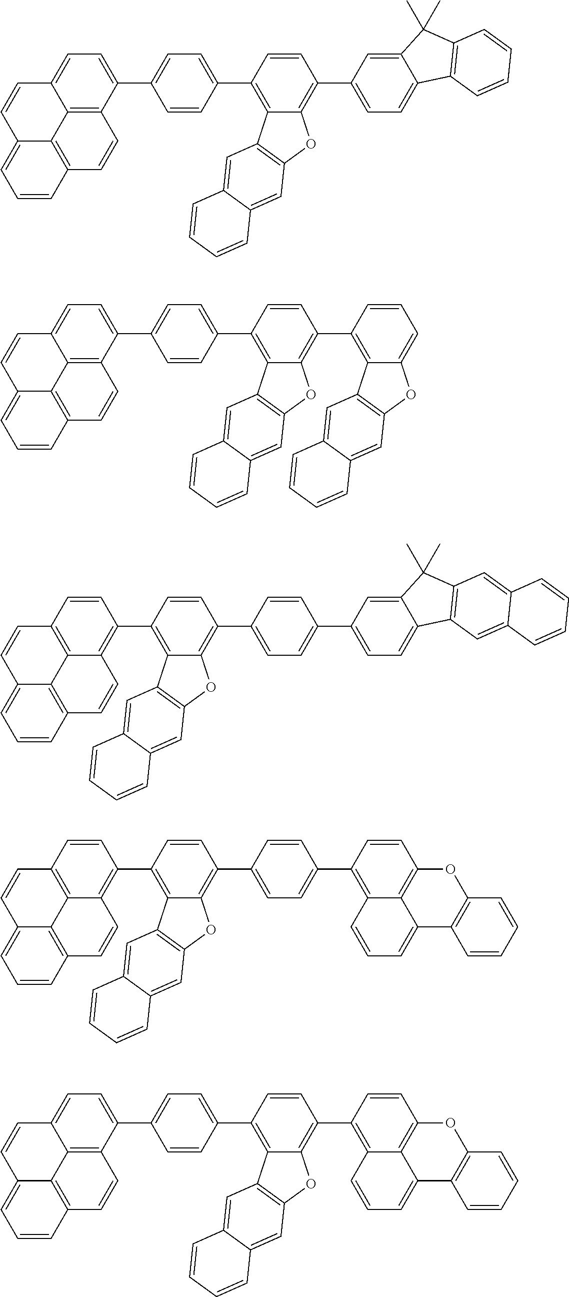

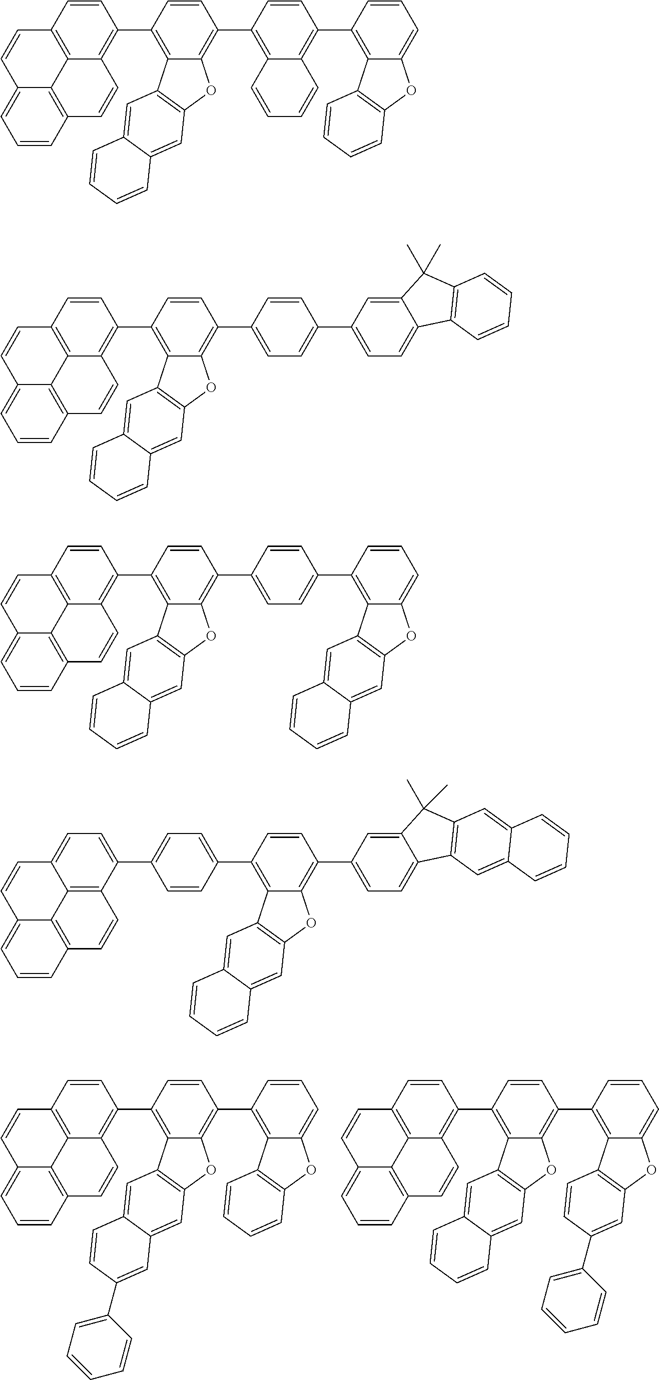

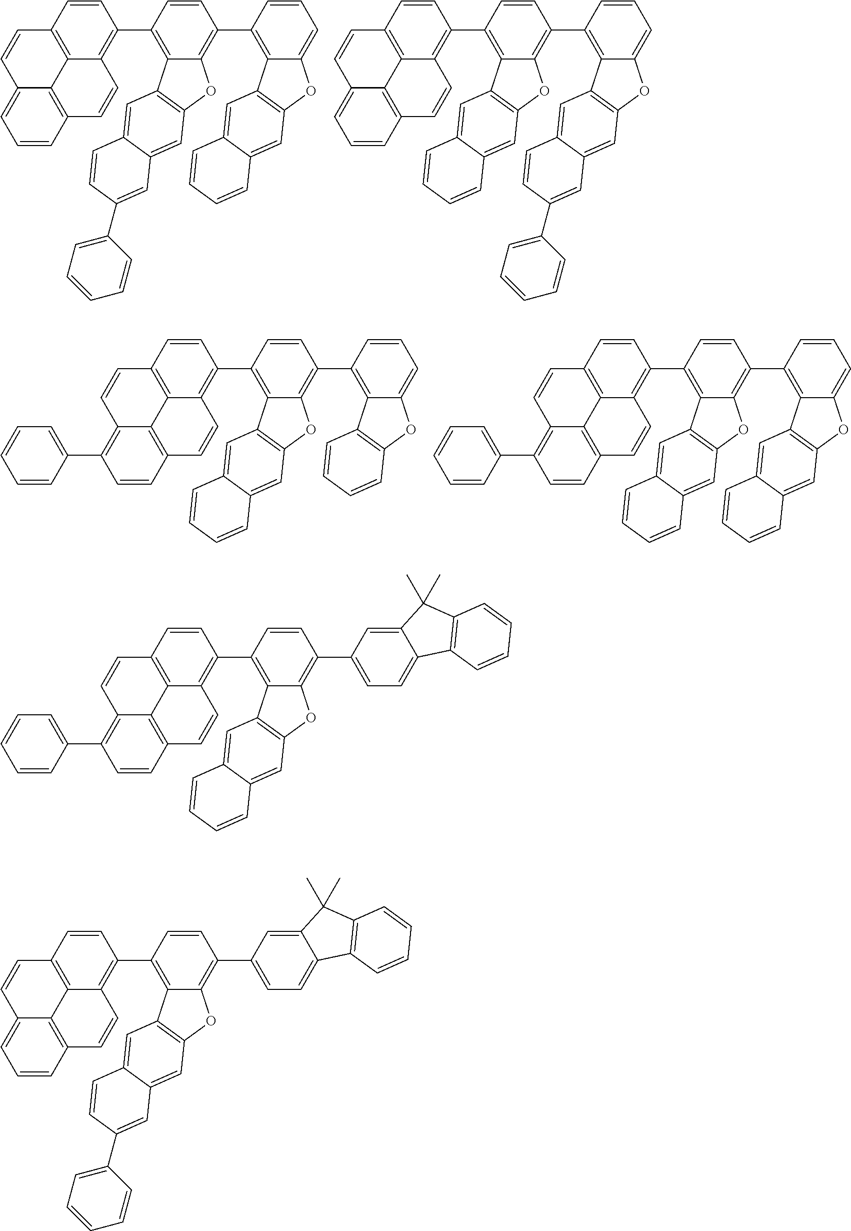

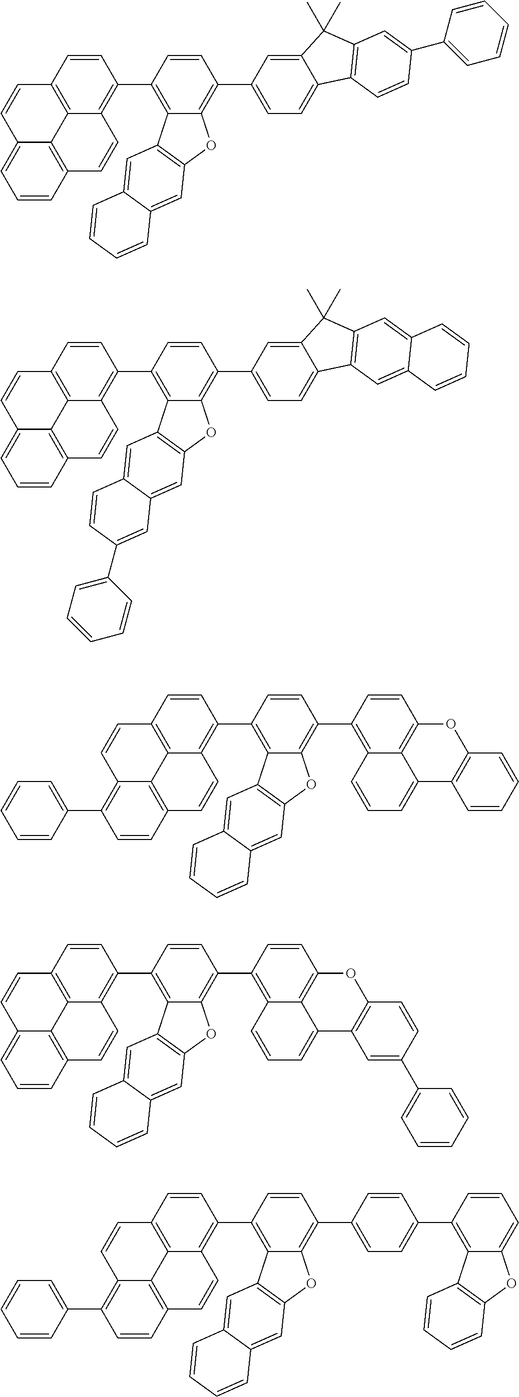 [SMILES: CC1(C)c2cc(-c3ccccc3)ccc2-c2ccc(-c3ccc(-c4ccc5ccc6cccc7ccc4c5c67)c4c3oc3cc5ccccc5cc34)cc21 ; CC1(C)c2cc(-c3ccc(-c4ccc5ccc6cccc7ccc4c5c67)c4c3oc3cc5cc(-c6ccccc6)ccc5cc34)ccc2-c2cc3ccccc3cc21 ; c1ccc(-c2ccc3ccc4c(-c5ccc(-c6ccc7c8c(cccc68)-c6ccccc6O7)c6oc7cc8ccccc8cc7c56)ccc5ccc2c3c54)cc1 ; c1ccc(-c2ccc3c(c2)-c2cccc4c(-c5ccc(-c6ccc7ccc8cccc9ccc6c7c89)c6c5oc5cc7ccccc7cc56)ccc(c24)O3)cc1 ; c1ccc(-c2ccc3ccc4c(-c5ccc(-c6ccc(-c7cccc8oc9ccccc9c78)cc6)c6oc7cc8ccccc8cc7c56)ccc5ccc2c3c54)cc1]

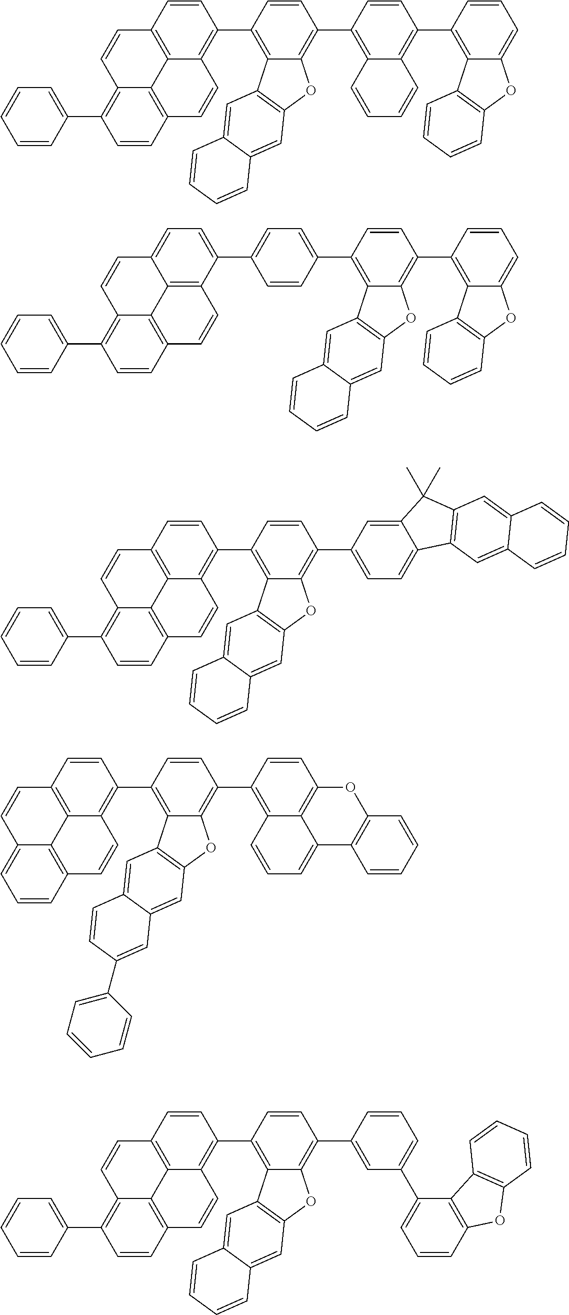

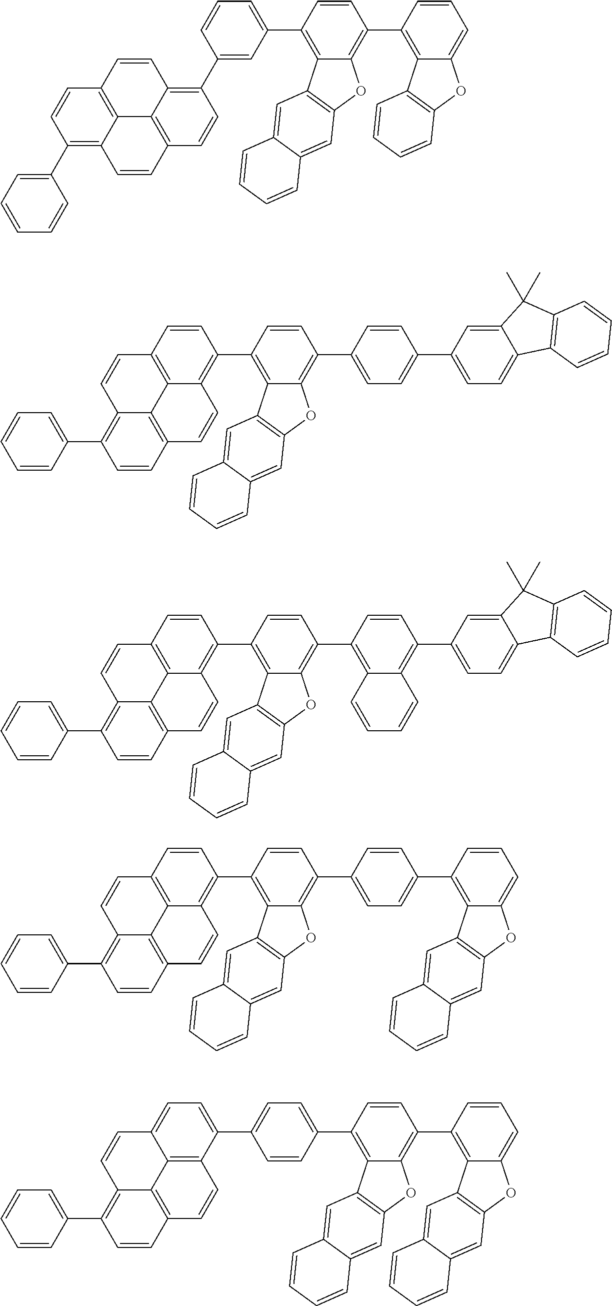

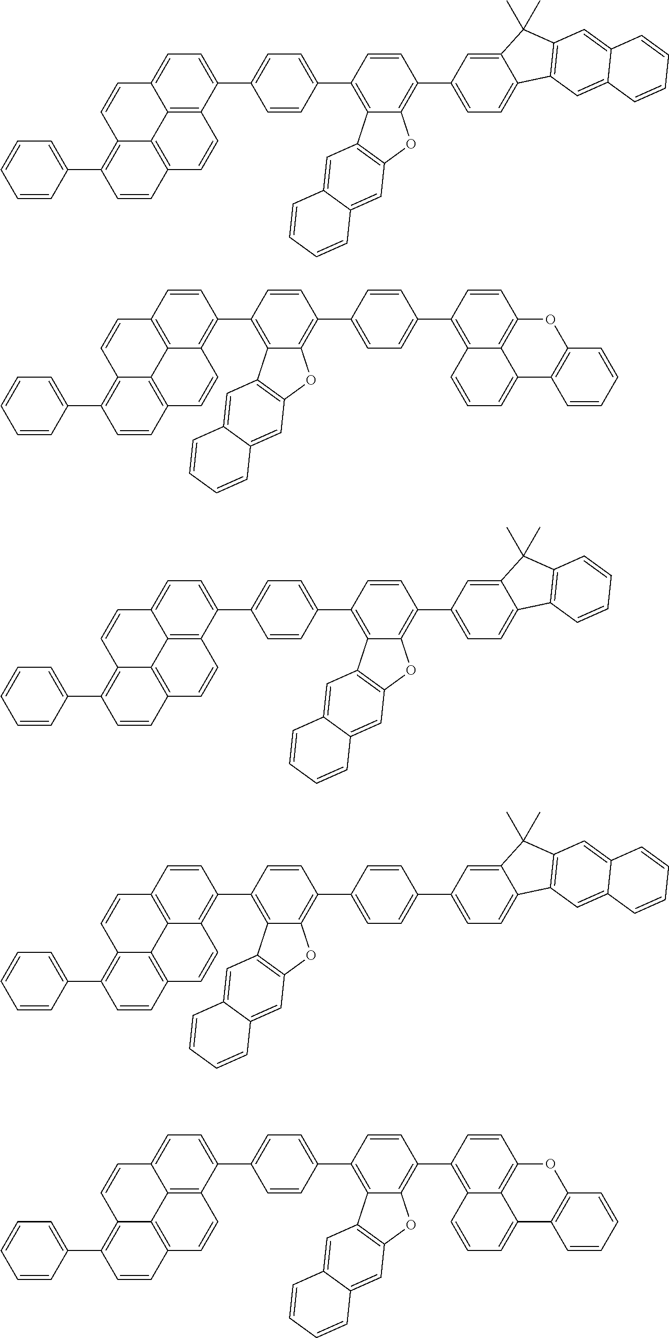

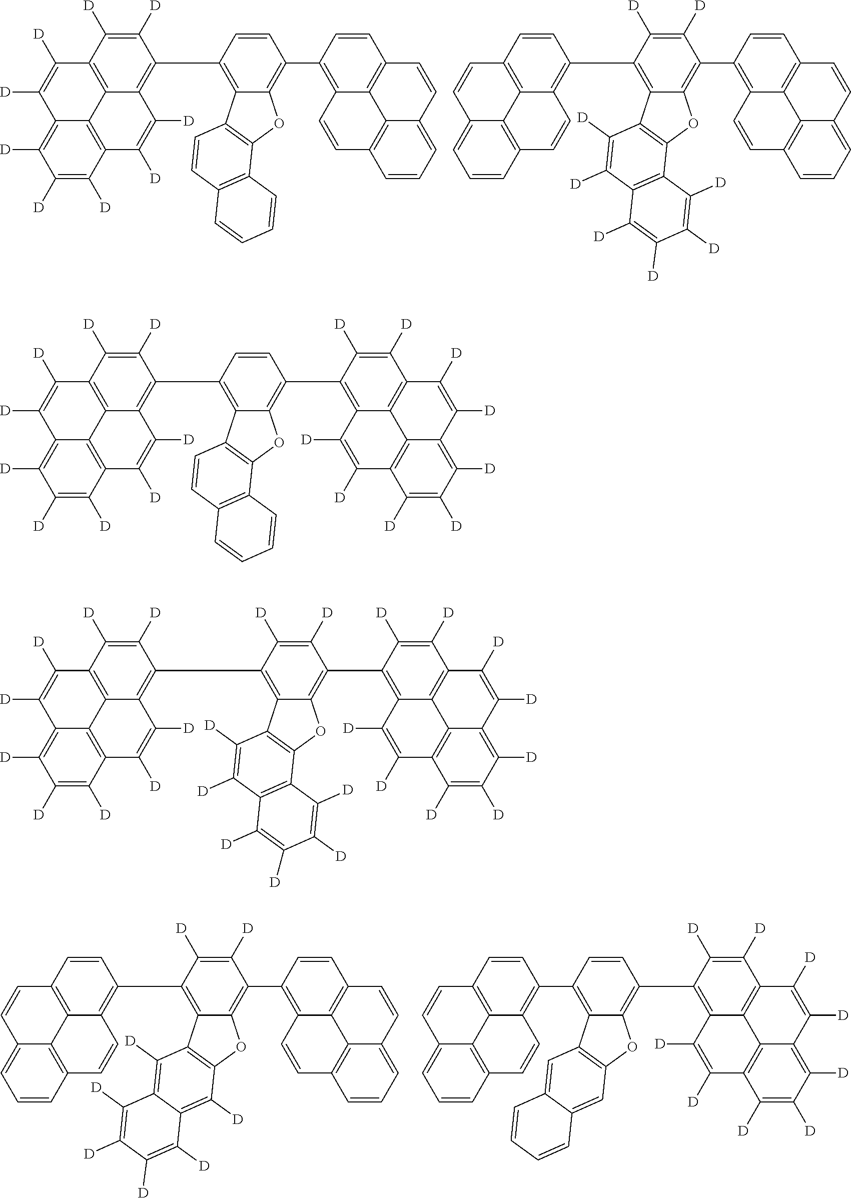

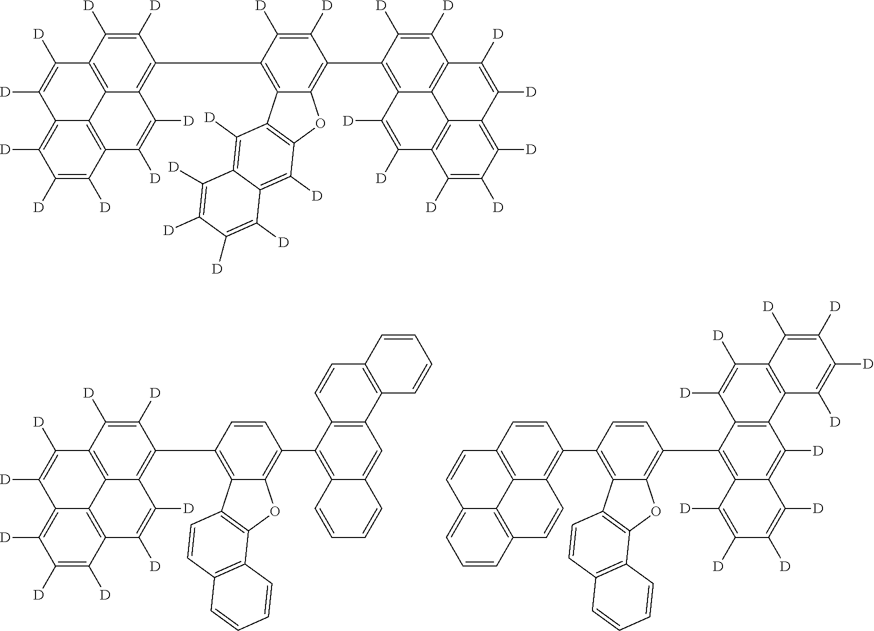

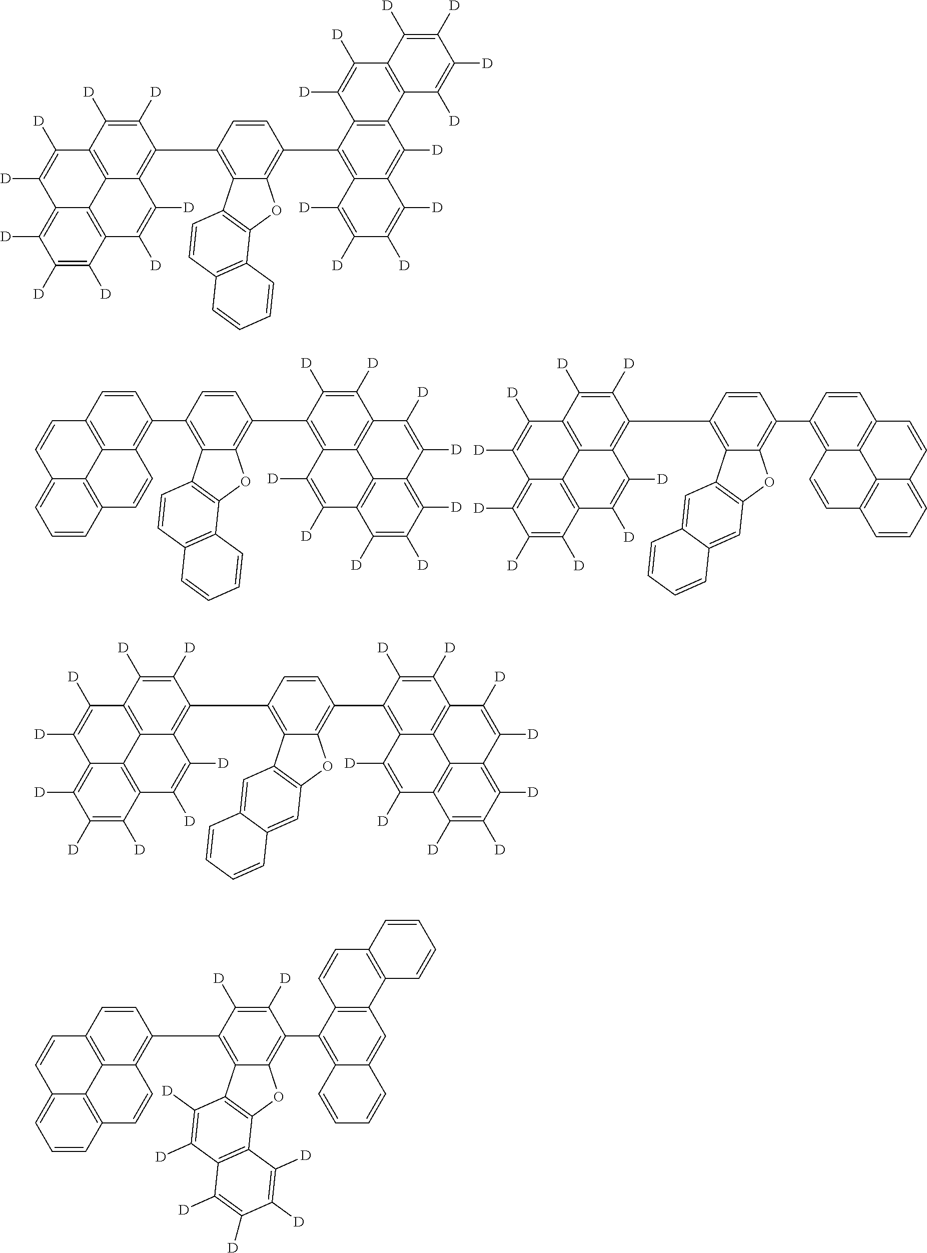

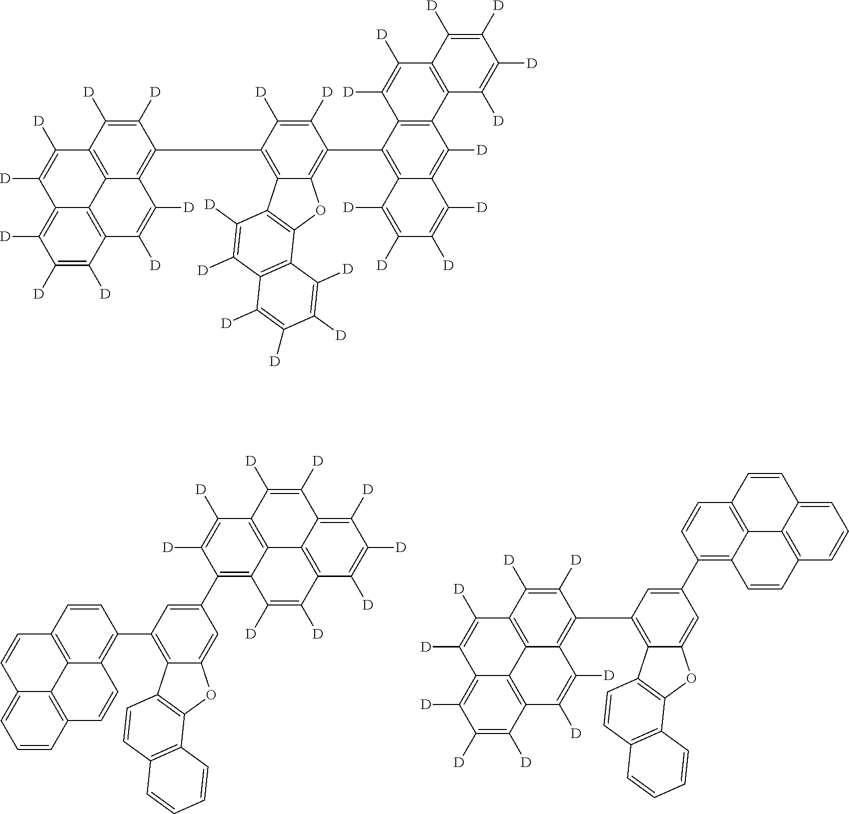

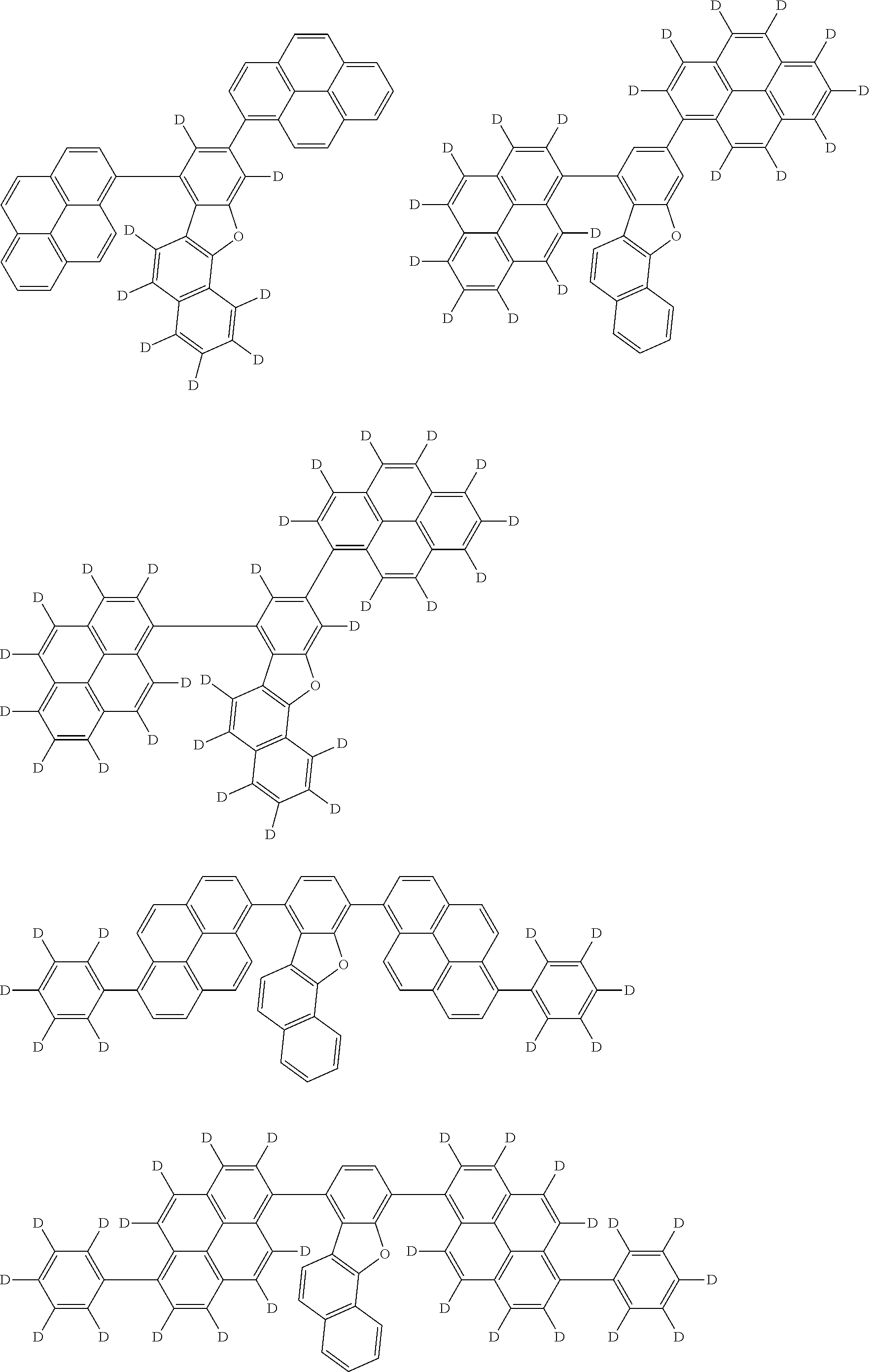

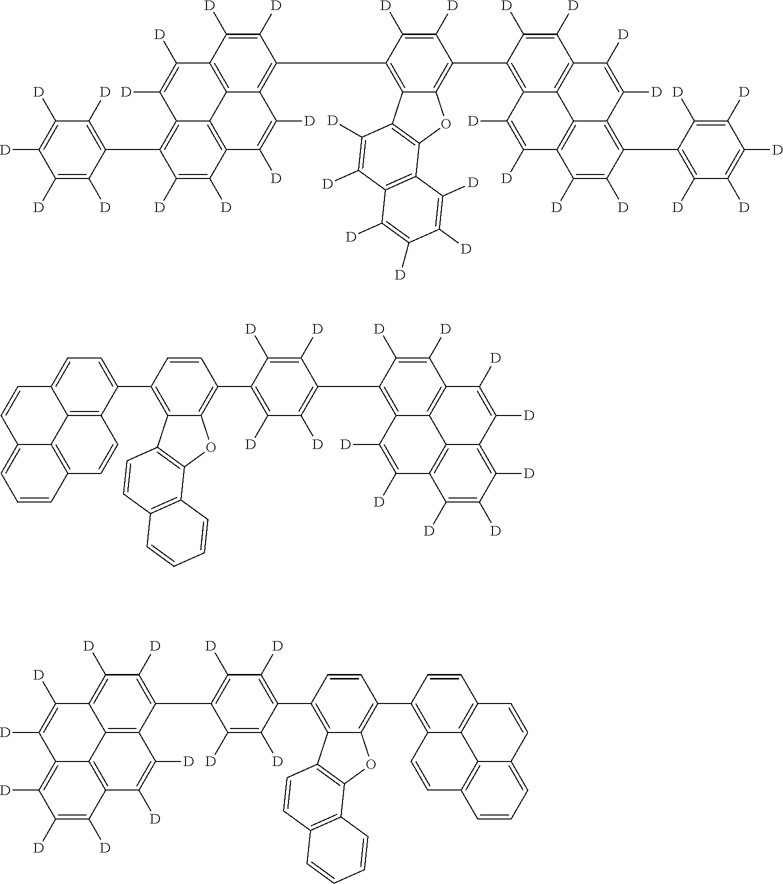

### Second Exemplary Embodiment

### Organic-Electroluminescence-Device Material

An organic-electroluminescence-device material according to a second exemplary embodiment contains a compound according to the first exemplary embodiment.

One example is an organic-electroluminescence-device material containing only a compound according to the first exemplary embodiment, and another example is an organic-electroluminescence-device material containing a compound according to the first exemplary embodiment and other compounds different from the compound according to the first exemplary embodiment.

In the organic-electroluminescence-device material according to the second exemplary embodiment, the compound according to the first exemplary embodiment is preferably a host material.

In this case, the organic-electroluminescence-device material may contain the compound according to the first exemplary embodiment as the host material and other compounds such as an emitting compound as a dopant material.

### Third Exemplary Embodiment

### Organic Electroluminescence Device

An organic electroluminescence device according to a third exemplary embodiment includes an anode, a cathode, and an emitting zone provided between the anode and the cathode.

A first emitting layer of the organic EL device according to the third exemplary embodiment contains a compound according to the first exemplary embodiment (compound represented by the formula (1)) as a first host material.

As described above, the compound according to the first exemplary embodiment has improved durability in an excited state and is less likely to undergo intermolecular interaction.

Therefore, according to the third exemplary embodiment, an organic EL device having a prolonged lifetime and improved chromaticity can be provided.

### Emitting Zone

The emitting zone of the organic EL device according to the third exemplary embodiment includes one or more emitting layers.

In the organic EL device according to the third exemplary embodiment, the emitting zone includes the first emitting layer.

In the organic EL device according to the third exemplary embodiment, the first emitting layer contains a compound according to the first exemplary embodiment as a first host material.

In an example of the organic EL device according to the third exemplary embodiment, the emitting zone includes only the first emitting layer.

The organic EL device according to the third exemplary embodiment may include the first emitting layer and in addition one or more organic layers.

In an example of the organic EL device according to the third exemplary embodiment, the emitting zone includes the first emitting layer and a second emitting layer.

### First Emitting Layer

In the organic EL device according to the third exemplary embodiment, the first emitting layer preferably contains the first host material and a first emitting compound.

In the organic EL device according to the third exemplary embodiment, the first emitting compound emits light with a maximum peak wavelength of preferably 500 nm or less, more preferably 480 nm or less, still more preferably 470 nm or less, and still further more preferably 465 nm or less.

In the organic EL device according to the third exemplary embodiment, the first emitting compound emits light with a maximum peak wavelength of preferably 430 nm or more, more preferably 440 nm or more, and still more preferably 445 nm or more.

In the organic EL device according to the third exemplary embodiment, the first emitting compound emits fluorescence with a maximum peak wavelength of preferably 500 nm or less, more preferably 480 nm or less, still more preferably 470 nm or less, and still further more preferably 465 nm or less.

In the organic EL device according to the third exemplary embodiment, the first emitting compound emits fluorescence with a maximum peak wavelength of preferably 430 nm or more, more preferably 440 nm or more, and still more preferably 445 nm or more.

In the organic EL device according to the third exemplary embodiment, the first emitting compound is preferably a compound not including an azine ring structure in a molecule.

In the organic EL device according to the third exemplary embodiment, the first emitting compound is preferably not a boron-containing complex, and the first emitting compound is more preferably not a complex.

In the organic EL device according to the third exemplary embodiment, the first emitting layer preferably contains no metal complex.

In the organic EL device according to the third exemplary embodiment, the first emitting layer also preferably contains no boron-containing complex.

In the organic EL device according to the third exemplary embodiment, the first emitting layer preferably contains no phosphorescent material (dopant material).

The first emitting layer preferably contains neither a heavy-metal complex nor a phosphorescent rare earth metal complex.

Examples of the heavy-metal complex herein include iridium complex, osmium complex, and platinum complex.

A method of measuring the maximum peak wavelength of the compound is as follows.

A toluene solution of a measurement target compound at a concentration of 5 μmol/L is prepared and put in a quartz cell, and an emission spectrum (ordinate axis: luminous intensity, abscissa axis: wavelength) of this sample is measured at normal temperature (300 K).

The emission spectrum can be measured using a spectrophotometer (machine name: F-7000) produced by Hitachi High-Tech Science Corporation.

It should be noted that the machine for measuring the emission spectrum is not limited to the machine used herein.

In the emission spectrum, the peak wavelength at a maximum luminous intensity is a maximum peak wavelength.

Herein, a maximum peak wavelength of fluorescence is occasionally referred to as a maximum fluorescence peak wavelength (FL-peak).

In an emission spectrum of the first emitting compound, when the height of a maximum peak, which is a peak at a maximum luminous intensity, is taken as 1, the height of other peaks appearing in the emission spectrum is preferably less than 0.6.

It should be noted that the peaks in the emission spectrum are defined as local maximum values.

In the emission spectrum of the first emitting compound, the number of peaks is preferably less than 3.

In the organic EL device according to the third exemplary embodiment, a triplet energy T₁(H1) of the first host material and a triplet energy T₁(D1) of the first emitting compound preferably satisfy a relationship of a numerical formula (Numerical Formula 6) below.

T₁(D1)>T₁(H1) (Numerical Formula 6)

When the first host material and the first emitting compound satisfy the relationship of the numerical formula (Numerical Formula 6), triplet excitons generated on the first host material do not transfer to the first emitting compound having a higher triplet energy.

Triplet excitons generated on the first emitting compound quickly energy-transfer to molecules of the first host material.

In other words, the triplet excitons on the first host material do not transfer to the first emitting compound and exhibit the TTF phenomenon in which the triplet excitons efficiently collide with one another on the first host material to generate singlet excitons.

In the organic EL device according to the third exemplary embodiment, a singlet energy S₁(H1) of the first host material and a singlet energy S₁ (D1) of the first emitting compound preferably satisfy a relationship of a numerical formula (Numerical Formula 5) below.

The singlet energy S₁ means an energy difference between the lowest singlet state and the ground state.

S₁(H1)>S₁(D1) (Numerical Formula 5)

When the first host material and the first emitting compound satisfy the relationship of the numerical formula (Numerical Formula 5), singlet excitons generated on the first host material easily energy-transfer from the first host material to the first emitting compound, thus contributing to fluorescence of the first emitting compound.

In the organic EL device according to the third exemplary embodiment, the first emitting compound is preferably contained at 0.5 mass % or more in the first emitting layer.

In other words, the first emitting layer contains the first emitting compound preferably at 0.5 mass % or more, more preferably at 1.0 mass % or more, still more preferably at 1.2 mass % or more, and still further more preferably at 1.5 mass % or more with respect to the total mass of the first emitting layer.

The first emitting layer contains the first emitting compound preferably at 10 mass % or less, more preferably at 7 mass % or less, and still more preferably at 5 mass % less with respect to the total mass of the first emitting layer.

In the organic EL device according to the third exemplary embodiment, the first emitting layer contains the first host material preferably at 60 mass % or more, more preferably at 70 mass % or more, still more preferably at 80 mass % or more, still further more preferably at 90 mass % or more, and yet still further more preferably at 95 mass % or more with respect to the total mass of the first emitting layer.

The first emitting layer preferably contains the first host material at 99 mass % or less with respect to the total mass of the first emitting layer.

When the first emitting layer contains the first host material and the first emitting compound, the upper limit of the total of the content ratios of the first host material and the first emitting compound is 100 mass %.

In the third exemplary embodiment, the first emitting layer may contain any other material than the first host material and the first emitting compound.

The first emitting layer may contain a single type of the first host material or may contain two or more types of the first host material.

The first emitting layer may contain a single type of the first emitting compound or may contain two or more types of the first emitting compound.

Second Emitting Layer

In the organic EL device according to the third exemplary embodiment, the emitting zone also preferably further includes the second emitting layer.

In the organic EL device according to the third exemplary embodiment, the second emitting layer preferably contains a second host material.

The second host material is a compound different from the first host material contained in the first emitting layer.

In the organic EL device according to the third exemplary embodiment, the second emitting layer preferably contains the second host material and a second emitting compound.

In the organic EL device according to the third exemplary embodiment, the first emitting compound and the second emitting compound are mutually the same or different.

In the organic EL device according to the third exemplary embodiment, the second emitting compound preferably emits light with a maximum peak wavelength of 500 nm or less.

The first emitting compound and the second emitting compound are each independently a compound that emits light with a maximum peak wavelength of 500 nm or less.

In the organic EL device according to the third exemplary embodiment, the second emitting compound emits light with a maximum peak wavelength of preferably 480 nm or less, more preferably 470 nm or less, and still more preferably 465 nm or less.

In the organic EL device according to the third exemplary embodiment, the second emitting compound emits light with a maximum peak wavelength of preferably 430 nm or more, more preferably 440 nm or more, and still more preferably 445 nm or more.

In the organic EL device according to the third exemplary embodiment, the second emitting compound emits fluorescence with a maximum peak wavelength of preferably 500 nm or less, more preferably 480 nm or less, still more preferably 470 nm or less, and still further more preferably 465 nm or less.

In the organic EL device according to the third exemplary embodiment, the second emitting compound emits fluorescence with a maximum peak wavelength of preferably 430 nm or more, more preferably 440 nm or more, and still more preferably 445 nm or more.

A method of measuring the maximum peak wavelength of the compound is as described above.

In the organic EL device according to the third exemplary embodiment, the full width at half maximum of a maximum peak of the second emitting compound is preferably in a range from 1 nm to 20 nm.

In the organic EL device according to the third exemplary embodiment, preferably, the emitting zone includes the first emitting layer and the second emitting layer, the first emitting layer contains the first host material and the first emitting compound, the second emitting layer contains the second host material and the second emitting compound, the first host material and the second host material are mutually different, and the first emitting compound and the second emitting compound are mutually the same or different.

In the organic EL device according to the third exemplary embodiment, the triplet energy T₁(H1) of the first host material and a triplet energy T₁(H2) of the second host material preferably satisfy a relationship of a numerical formula (Numerical Formula 1) below.

T₁(H1)>T₁(H2) (Numerical Formula 1)

According to an exemplary arrangement of the third exemplary embodiment, an organic electroluminescence device with improved luminous efficiency can be provided.

Triplet-triplet-annihilation (occasionally referred to as TTA) is conventionally known as a technique for improving the luminous efficiency of an organic electroluminescence device.

TTA is a mechanism in which triplet excitons collide with one another to generate singlet excitons.

The TTA mechanism is occasionally referred to as the TTF mechanism as described in International Publication No. 2010/134350.

The TTF phenomenon will be described.

Holes injected from an anode and electrons injected from a cathode are recombined in an emitting layer to generate excitons.

As for the spin state, as is conventionally known, singlet excitons account for 25% and triplet excitons account for 75%.

In a conventionally known fluorescent device, light is emitted when singlet excitons of 25% are relaxed to the ground state. The remaining triplet excitons of 75% are returned to the ground state without emitting light through a thermal deactivation process.

Accordingly, the theoretical limit value of the internal quantum efficiency of the conventional fluorescent device is believed to be 25%.

The behavior of triplet excitons generated within an organic substance has been theoretically examined.

According to S. M. Bachilo et al. (J. Phys. Chem. A, 104, 7711 (2000)), assuming that high-order excitons such as quintet excitons are quickly returned to triplet excitons, triplet excitons (hereinafter abbreviated as ³A*) collide with one another with an increase in density thereof, whereby a reaction shown by the following formula occurs.

In the formula, ¹A represents the ground state and ¹A* represents the lowest singlet excitons.

³A*+³A*→( 4/9)¹A+( 1/9)¹A*+(13/9)³A*

In other words, 5³A*→4¹A+1A* is satisfied, and it is expected that, among triplet excitons initially generated, which account for 75%, one fifth thereof (i.e., 20%) is changed to singlet excitons.

Accordingly, the amount of singlet excitons which contribute to emission is 40%, which is a value obtained by adding 15% (75%×(⅕)=15%) to 25%, which is the amount ratio of initially generated singlet excitons.

At this time, a ratio of luminous intensity derived from TTF (TTF ratio) relative to the total luminous intensity is 15/40, i.e., 37.5%.

Assuming that singlet excitons are generated by collision of initially generated triplet excitons accounting for 75% (i.e., one singlet exciton is generated from two triplet excitons), a significantly high internal quantum efficiency of 62.5% is obtained, which is a value obtained by adding 37.5% (75%×(½)=37.5%) to 25% (the amount ratio of initially generated singlet excitons).

At this time, the TTF ratio is 37.5/62.5=60%.

In the organic electroluminescence device according to the third exemplary embodiment, triplet excitons generated in the first emitting layer through recombination of holes and electrons are considered to be less likely to be quenched at the interface between the first emitting layer and an organic layer in direct contact with the first emitting layer even if carriers are excessively present at the interface.

For instance, the presence of a recombination region locally on an interface between the first emitting layer and a hole transporting layer or an electron blocking layer is considered to cause quenching by excessive electrons.

Meanwhile, the presence of a recombination region locally on an interface between the first emitting layer and an electron transporting layer or a hole blocking layer is considered to cause quenching by excessive holes.

The organic electroluminescence device according to the third exemplary embodiment includes at least two emitting layers (i.e., the first emitting layer and the second emitting layer) satisfying a predetermined relationship, and the triplet energy T₁(H1) of the first host material in the first emitting layer and the triplet energy T₁(H2) of the second host material in the second emitting layer satisfy the relationship of the numerical formula (Numerical Formula 1).

Because of the presence of the first emitting layer and the second emitting layer satisfying the relationship of the numerical formula (Numerical Formula 1), triplet excitons generated in the first emitting layer can transfer to the second emitting layer without being quenched by excessive carriers and can be inhibited from back-transferring from the second emitting layer to the first emitting layer.

Consequently, the second emitting layer exhibits the TTF mechanism to effectively generate singlet excitons, thereby improving the luminous efficiency.

With this configuration in which the organic electroluminescence device includes, as different regions, the first emitting layer that mainly generates triplet excitons and the second emitting layer that mainly exhibits the TTF mechanism by utilizing the triplet excitons transferred from the first emitting layer and in which a compound having a triplet energy smaller than that of the first host material in the first emitting layer is used as the second host material in the second emitting layer to make a difference in triplet energy, the luminous efficiency is improved.

In the organic EL device according to the third exemplary embodiment, the triplet energy T₁(H1) of the first host material and the triplet energy T₁(H2) of the second host material preferably satisfy the relationship of a numerical formula (Numerical Formula 1B) below.

T₁(H1)−T₁(H2)>0.03 eV (Numerical Formula 1B)

Herein, the “host material” refers to, for instance, a material that accounts for “50 mass % or more of the layer”.

That is, for instance, the first emitting layer contains 50 mass % or more of the first host material with respect to the total mass of the first emitting layer.

For instance, the second emitting layer contains 50 mass % or more of the second host material with respect to the total mass of the second emitting layer.

In the organic EL device according to the third exemplary embodiment, when the emitting zone includes the first emitting layer and the second emitting layer and the first host material and the first emitting compound satisfy the relationship of the numerical formula (Numerical Formula 6), triplet excitons generated in the first emitting layer transfer not on the first emitting compound having a higher triplet energy but on the first host material, and thus easily transfer to the second emitting layer.

The organic EL device according to the third exemplary embodiment, when the emitting zone includes the first emitting layer and the second emitting layer, preferably satisfies a relationship of a numerical formula (Numerical Formula 20B) below.

T₁(D1)>T₁(H1)>T₁(H2) (Numerical Formula 20B)

In the organic EL device according to the third exemplary embodiment, a singlet energy S₁(H2) of the second host material and a singlet energy S₁(D2) of the second emitting compound preferably satisfy a relationship of a numerical formula (Numerical Formula 7) below.

S₁(H2)>S₁(D2) (Numerical Formula 7)

In the organic EL device according to the third exemplary embodiment, when the second emitting compound and the second host material satisfy the relationship of the numerical formula (Numerical Formula 7), since the singlet energy of the second emitting compound is smaller than the singlet energy of the second host material, singlet excitons generated by the TTF phenomenon energy-transfer from the second host material to the second emitting compound, thus contributing to fluorescence of the second emitting compound.

In the organic EL device according to the third exemplary embodiment, a triplet energy T₁(D2) of the second emitting compound and the triplet energy T₁(H2) of the second host material preferably satisfy a relationship of a numerical formula (Numerical Formula 8) below.

T₁(D2)>T₁(H2) (Numerical Formula 8)

In the organic EL device according to the third exemplary embodiment, when the second emitting compound and the second host material satisfy the relationship of the numerical formula (Numerical Formula 8), triplet excitons generated in the first emitting layer energy-transfer not to the second emitting compound having a higher triplet energy but to molecules of the second host material when transferring to the second emitting layer.

In addition, triplet excitons generated by recombination of holes and electrons on the second host material do not transfer to the second emitting compound having higher triplet energy.

Triplet excitons generated by recombination on molecules of the second emitting compound quickly energy-transfer to molecules of the second host material.

Triplet excitons in the second host material do not transfer to the second emitting compound but efficiently collide with one another on the second host material to generate singlet excitons by the TTF phenomenon.

In the organic EL device according to the third exemplary embodiment, the second emitting compound is preferably a compound not including an azine ring structure in a molecule.

In the organic EL device according to the third exemplary embodiment, the second emitting compound is preferably not a boron-containing complex, and the second emitting compound is more preferably not a complex.

In the organic EL device according to the third exemplary embodiment, the second emitting layer preferably contains no metal complex.

In the organic EL device according to the third exemplary embodiment, the second emitting layer also preferably contains no boron-containing complex.

In the organic EL device according to the third exemplary embodiment, the second emitting layer preferably contains no phosphorescent material (dopant material).

The second emitting layer preferably contains neither a heavy-metal complex nor a phosphorescent rare earth metal complex.

Examples of the heavy-metal complex herein include iridium complex, osmium complex, and platinum complex.

In the organic EL device according to the third exemplary embodiment, the second emitting compound is preferably contained at 0.5 mass % or more in the second emitting layer.

In other words, the second emitting layer contains the second emitting compound preferably at 0.5 mass % or more, more preferably at 1.0 mass % or more, still more preferably at 1.2 mass % or more, and still further more preferably at 1.5 mass % or more with respect to the total mass of the second emitting layer.

The second emitting layer contains the second emitting compound preferably at 10 mass % or less, more preferably at 7 mass % or less, and still more preferably at 5 mass % less with respect to the total mass of the second emitting layer.

The second emitting layer contains a second compound as the second host material preferably at 60 mass % or more, more preferably at 70 mass % or more, still more preferably at 80 mass % or more, still further more preferably at 90 mass % or more, and yet still further more preferably at 95 mass % or more, with respect to the total mass of the second emitting layer.

The second emitting layer preferably contains the second host material at 99 mass % or less with respect to the total mass of the second emitting layer.

When the second emitting layer contains the second host material and the second emitting compound, the upper limit of the total of the content ratios of the second host material and the second emitting compound is 100 mass %.

In the third exemplary embodiment, the second emitting layer may contain any other material than the second host material and the second emitting compound.

The second emitting layer may contain a single type of the second host material or may contain two or more types of the second host material.

The second emitting layer may contain a single type of the second emitting compound or may contain two or more types of the second emitting compound.

In the organic EL device according to the third exemplary embodiment, a triplet energy T₁(DX) of the first emitting compound or the second emitting compound, the triplet energy T₁(H1) of the first host material, and the triplet energy T₁(H2) of the second host material preferably satisfy a relationship of a numerical formula (Numerical Formula 10) below.

2.6 eV>T₁(DX)>T₁(H1)>T₁(H2) (Numerical Formula 10)

The triplet energy T₁(D1) of the first emitting compound preferably satisfies a relationship of a numerical formula (Numerical Formula 10A) below.

2.6 eV>T₁(D1)>T₁(H1)>T₁(H2) (Numerical Formula 10A)

The triplet energy T₁(D2) of the second emitting compound preferably satisfies a relationship of a numerical formula (Numerical Formula 10B) below.

2.6 eV>T₁(D2)>T₁(H1)>T₁(H2) (Numerical Formula 10B)

In the organic EL device according to the third exemplary embodiment, the triplet energy T₁(DX) of the first emitting compound or the second emitting compound and the triplet energy T₁(H1) of the first host material preferably satisfy the relationship of a numerical formula (Numerical Formula 11) below.

0 eV<T₁(DX)−T₁(H1)<0.6 eV (Numerical Formula 11).

The triplet energy T₁(D1) of the first emitting compound preferably satisfies the relationship of a numerical formula (Numerical Formula 11A) below.

0 eV<T₁(D1)−T₁(H1)<0.6 eV (Numerical Formula 11A)

The triplet energy T₁(D2) of the second emitting compound preferably satisfies the relationship of a numerical formula (Numerical Formula 11B) below.

0 eV<T₁(D2)−T₁(H2)<0.8 eV (Numerical Formula 11B)

In the organic EL device according to the third exemplary embodiment, the triplet energy T₁(H1) of the first host material preferably satisfies the relationship of a numerical formula (Numerical Formula 12) below.

T₁(H1)>2.0 eV (Numerical Formula 12).

In the organic EL device according to the third exemplary embodiment, the triplet energy T₁(H1) of the first host material also preferably satisfies a relationship of a numerical formula (Numerical Formula 12A) below, and also preferably satisfies a relationship of a numerical formula (Numerical Formula 12B) below.

T₁(H1)>2.10 eV (Numerical Formula 12A).

T₁(H1)>2.15 eV (Numerical Formula 12B)

In the organic EL device according to the third exemplary embodiment, when the triplet energy T₁(H1) of the first host material satisfies the relationship of the numerical formula (Numerical Formula 12A) or the numerical formula (Numerical Formula 12B), triplet excitons generated in the first emitting layer easily transfer to the second emitting layer and are easily inhibited from back-transferring from the second emitting layer to the first emitting layer.

Consequently, singlet excitons are efficiently generated in the second emitting layer, thereby improving luminous efficiency.

In the organic EL device according to the third exemplary embodiment, the triplet energy T₁(H1) of the first host material also preferably satisfies a relationship of a numerical formula (Numerical Formula 12C) below, and also preferably satisfies a relationship of a numerical formula (Numerical Formula 12D) below.

2.08 eV>T₁(H1)>1.87 eV (Numerical Formula 12C).

2.05 eV>T₁(H1)>1.90 eV (Numerical Formula 12D).

In the organic EL device according to the third exemplary embodiment, when the triplet energy T₁(H1) of the first host material satisfies the relationship of the numerical formula (Numerical Formula 12C) or the numerical formula (Numerical Formula 12D), the energy of triplet excitons generated in the first emitting layer is small, which is expected to prolong the lifetime of the organic EL device.

In the organic EL device according to the third exemplary embodiment, the triplet energy T₁(D1) of the first emitting compound also preferably satisfies a relationship of a numerical formula (Numerical Formula 14A) below, and also preferably satisfies a relationship of a numerical formula (Numerical Formula 14B) below.

2.60 eV>T₁(D1) (Numerical Formula 14A)

2.50 eV>T₁(D1) (Numerical Formula 14B)

When the first emitting layer contains the first emitting compound satisfying the relationship of the numerical formula (Numerical Formula 14A) or (Numerical Formula 14B), the organic EL device has a prolonged lifetime.

In the organic EL device according to the third exemplary embodiment, the triplet energy T₁(D2) of the second emitting compound also preferably satisfies a relationship of a numerical formula (Numerical Formula 14C) below, and also preferably satisfies a relationship of a numerical formula (Numerical Formula 14D) below.

2.60 eV>T₁(D2) (Numerical Formula 14C)

2.50 eV>T₁(D2) (Numerical Formula 14D)

When the second emitting layer contains a compound satisfying the relationship of the numerical formula (Numerical Formula 14C) or (Numerical Formula 14D), the organic EL device has a prolonged lifetime.

In the organic EL device according to the third exemplary embodiment, the triplet energy T₁(H2) of the second host material also preferably satisfies a relationship of a numerical formula (Numerical Formula 13) below.

T₁(H2)>1.9 eV (Numerical Formula 13)

In the organic EL device according to the third exemplary embodiment, the triplet energy T₁(H2) of the second host material also preferably satisfies a relationship of a numerical formula (Numerical Formula 13A) below.

1.9 eV≥T₁(H2)≥1.8 eV (Numerical Formula 13A)

In the organic EL device according to the third exemplary embodiment, the first emitting layer is also preferably provided between the anode and the second emitting layer.

In the organic EL device according to the third exemplary embodiment, the second emitting layer is also preferably provided between the anode and the first emitting layer.

In the organic EL device according to the third exemplary embodiment, one of the first emitting layer and the second emitting layer is preferably a layer provided closest to the anode among a plurality of layers included in the emitting zone.

In the organic EL device according to the third exemplary embodiment, one of the first emitting layer and the second emitting layer is preferably a layer provided closest to the cathode among the plurality of layers included in the emitting zone.

The organic EL device according to the third exemplary embodiment may include the anode, the first emitting layer, the second emitting layer, and the cathode in this order, or the order of the first emitting layer and the second emitting layer may be reversed.

In other words, the organic EL device may include the anode, the second emitting layer, the first emitting layer, and the cathode in this order.

Regardless of the order of the first emitting layer and the second emitting layer, the effect of a layered structure of the first emitting layer and the second emitting layer is expected to be exhibited by selecting a combination of materials satisfying the relationship of the numerical formula (Numerical Formula 1).

In the organic EL device according to the third exemplary embodiment, when the first emitting layer and the second emitting layer are layered in this order from the anode side, an electron mobility μe(H1) of the first host material and an electron mobility μe(H2) of the second host material also preferably satisfy a relationship of a numerical formula (Numerical Formula 30) below.

μe(H2)>μe(H1) (Numerical Formula 30)

When the first host material and the second host material satisfy the relationship of the numerical formula (Numerical Formula 30), a recombination ability between holes and electrons in the first emitting layer is improved.

In the organic EL device according to the third exemplary embodiment, when the first emitting layer and the second emitting layer are layered in this order from the anode side, a hole mobility μh(H1) of the first host material and a hole mobility μh(H2) of the second host material also preferably satisfy a relationship of a numerical formula (Numerical Formula 31) below.

μh(H1)>μh(H2) (Numerical Formula 31)

In the organic EL device according to the third exemplary embodiment, when the first emitting layer and the second emitting layer are layered in this order from the anode side, the hole mobility μh(H1) of the first host material, the electron mobility μe(H1) of the first host material, the hole mobility μh(H2) of the second host material, and the electron mobility μe(H2) of the second host material also preferably satisfy a relationship of a numerical formula (Numerical Formula 32) below.

(μe(H2)/μh(H2))>(μe(H1)/μh(H1)) (Numerical Formula 32)

The electron mobility can be measured by performing an impedance measurement using a device for mobility evaluation produced according to the following procedure.

The device for mobility evaluation is produced, for instance, according to the following steps.

A compound Target to be measured for electron mobility is vapor-deposited on a glass substrate having an aluminum electrode (anode) so as to cover the aluminum electrode, thereby forming a measurement target layer.

A compound ET-A below is vapor-deposited on the measurement target layer to form an electron transporting layer.

LiF is vapor-deposited on the electron transporting layer to form an electron injecting layer.

Metal aluminum (Al) is vapor-deposited on the electron injecting layer to form a metal cathode.

An arrangement of the above device for mobility evaluation is roughly shown as follows.

Glass/Al (50)/Target (200)/ET-A (10)/LiF (1)/Al (50)

Numerals in parentheses represent a film thickness (nm).

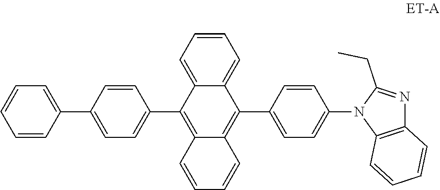 [SMILES: CCc1nc2ccccc2n1-c1ccc(-c2c3ccccc3c(-c3ccc(-c4ccccc4)cc3)c3ccccc23)cc1]

The device for electron mobility evaluation is set in an impedance measurement apparatus, and an impedance measurement is performed.

In the impedance measurement, a measurement frequency is swept from 1 Hz to 1 MHz.

At this time, an alternating current amplitude of 0.1 V and a direct current voltage V are applied to the device.

A modulus M is calculated from a measured impedance Z using a relationship of a calculation formula (C1) below.

M=jωZ Calculation Formula (C1):

In the calculation formula (C1), j is an imaginary unit whose square is −1 and ω is an angular frequency [rad/s].

In a bode plot in which an imaginary part of the modulus M is represented by an ordinate axis and the frequency [Hz] is represented by an abscissa axis, an electrical time constant τ of the device for mobility evaluation is obtained from a frequency fmax showing a peak using a calculation formula (C2) below.

τ=1/(2πfmax) Calculation Formula (C2):

τ in the calculation formula (C2) is a symbol representing a circumference ratio.

Using τ, an electron mobility μe is calculated from a relationship of a calculation formula (C3-1) below.

μe=d²/(Vτ) Calculation Formula (C3-1):

In the calculation formula (C3-1), d is a total film thickness of organic thin film(s) forming the device. In a case of the device arrangement for electron mobility evaluation, d=210 [nm] is satisfied.

Hole mobility can be measured by measuring impedance using a device for mobility evaluation produced according to the following steps.

The device for mobility evaluation is produced, for instance, according to the following steps.

A compound HA-2 below is vapor-deposited on a glass substrate having an ITO transparent electrode (anode) so as to cover the transparent electrode, thereby forming a hole injecting layer.

A compound HT-A is vapor-deposited on the hole injecting layer to form a hole transporting layer.

Subsequently, a compound Target, which is to be measured for the hole mobility, is vapor-deposited to form a measurement target layer.

Metal aluminum (Al) is vapor-deposited on the measurement target layer to form a metal cathode.

An arrangement of the above device for mobility evaluation is roughly shown as follows.

ITO(130)/HA-2(5)/HT-A(10)/Target(200)/Al(80)

Numerals in parentheses represent a film thickness (nm).

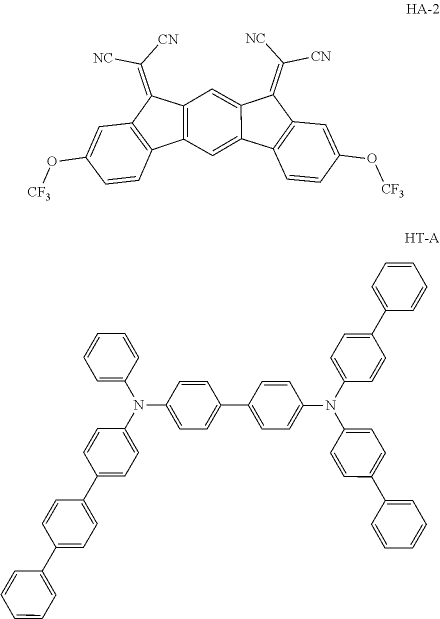 [SMILES: N#CC(C#N)=C1c2cc(OC(F)(F)F)ccc2-c2cc3c(cc21)C(=C(C#N)C#N)c1cc(OC(F)(F)F)ccc1-3 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

The device for evaluating hole mobility is set in an impedance measurement apparatus and an impedance measurement is performed.

In the impedance measurement, a measurement frequency is swept from 1 Hz to 1 MHz.

At this time, an alternating current amplitude of 0.1 V and a direct current voltage V are applied to the device.

A modulus M is calculated from a measured impedance Z using the relationship of the calculation formula (C1).

In a bode plot in which the ordinate axis represents the imaginary part of the modulus M and the abscissa axis represents frequency [Hz], an electrical time constant τ of the device for mobility evaluation is determined from a frequency fmax showing a peak by using the calculation formula (C2).

Using τ determined using the calculation formula (C2), a hole mobility μh is calculated from a relationship of a calculation formula (C3-2) below.

μh=d²/(Vτ) Calculation Formula (C3-2):

d in the calculation formula (C3-2) is a total film thickness of organic thin film(s) forming the device. In a case of the device arrangement for hole mobility evaluation, d=215 [nm] is satisfied.

The electron mobility and the hole mobility herein are values in the case where the square root of an electric field intensity, E^{1/2}, is 500 [V^{1/2}/cm^{1/2}].

The square root of the electric field intensity, E^{1/2}, can be calculated from a relationship of a calculation formula (C4) below.

E^{1/2}=V^{1/2}/d^{1/2} Calculation Formula (C4):

For the impedance measurement, a 1260 type by Solartron Analytical is used as the impedance measurement apparatus, and for a higher accuracy, a 1296 type dielectric constant measurement interface by Solartron Analytical can be used together therewith.

In the organic EL device according to the third exemplary embodiment, the first emitting layer and the second emitting layer are preferably in direct contact with each other.

Herein, the layer structure in which “the first emitting layer and the second emitting layer are in direct contact with each other” may include, for instance, any of the following embodiments (LS1), (LS2), and (LS3).

(LS1) An embodiment in which a region containing both the first host material and the second host material is generated in a process of vapor-depositing the compound of the first emitting layer and vapor-depositing the compound of the second emitting layer, and is present on the interface between the first emitting layer and the second emitting layer.

(LS2) An embodiment in which in a case of containing an emitting compound in the first emitting layer and the second emitting layer, a region containing the first host material, the second host material and the emitting compound is generated in a process of vapor-depositing the compound of the first emitting layer and vapor-depositing the compound of the second emitting layer, and is present on the interface between the first emitting layer and the second emitting layer.

(LS3) An embodiment in which in a case of containing an emitting compound in the first emitting layer and the second emitting layer, a region containing the emitting compound, a region containing the first host material or a region containing the second host material is generated in a process of vapor-depositing the compound of the first emitting layer and vapor-depositing the compound of the second emitting layer, and is present on the interface between the first emitting layer and the second emitting layer.

### Second Host Material

In the organic EL device according to the third exemplary embodiment, the second host material is, for instance, but is not limited to, a second compound represented by a formula (2) below.

### Second Compound

In the organic EL device according to the third exemplary embodiment, the second compound is preferably a compound represented by the formula (2) below.

The second host material is preferably the second compound represented by the formula (2) below.

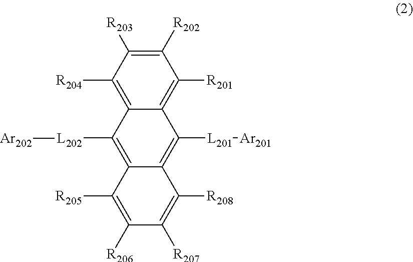

In the formula (2):
R₂₀₁ to R₂₀₈ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
L₂₀₁ and L₂₀₂ are each independently a single bond, a substituted or unsubstituted arylene group having 6 to 50 ring carbon atoms, or a substituted or unsubstituted divalent heterocyclic group having 5 to 50 ring atoms; and
Ar₂₀₁ and Ar₂₀₂ are each independently a substituted or unsubstituted aryl group having 6 to 50 ring carbon atoms, or a substituted or unsubstituted heterocyclic group having 5 to 50 ring atoms.
In the second host material, R₉₀₁, R₉₀₂, R₉₀₃, R₉₀₄, R₉₀₅, R₉₀₆, R₉₀₇, R₈₀₁, and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

In the organic EL device according to the third exemplary embodiment, R₂₀₁ to R₂₀₈ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, or a nitro group;
L₂₀₁ and L₂₀₂ are each independently a single bond, a substituted or unsubstituted arylene group having 6 to 50 ring carbon atoms, or a substituted or unsubstituted divalent heterocyclic group having 5 to 50 ring atoms;
Ar₂₀₁ and Ar₂₀₂ are each independently a substituted or unsubstituted aryl group having 6 to 50 ring carbon atoms, or a substituted or unsubstituted heterocyclic group having 5 to 50 ring atoms.

In the organic EL device according to the third exemplary embodiment, L₂₀₁ and L₂₀₂ are each independently a single bond or a substituted or unsubstituted arylene group having 6 to 50 ring carbon atoms, and Ar₂₀₁ and Ar₂₀₂ are preferably each independently a substituted or unsubstituted aryl group having 6 to 50 ring carbon atoms.

In the organic EL device according to the third exemplary embodiment, Ar₂₀₁ and Ar₂₀₂ are preferably each independently a phenyl group, naphthyl group, phenanthryl group, biphenyl group, terphenyl group, diphenylfluorenyl group, dimethylfluorenyl group, benzodiphenylfluorenyl group, benzodimethylfluorenyl group, dibenzofuranyl group, dibenzothienyl group, naphthobenzofuranyl group, or naphthobenzothienyl group.

In the organic EL device according to the third exemplary embodiment, the second compound represented by the formula (2) is preferably a compound represented by a formula (201), a formula (202), a formula (203), a formula (204), a formula (205), a formula (206), a formula (207), a formula (208), or a formula (209) below.

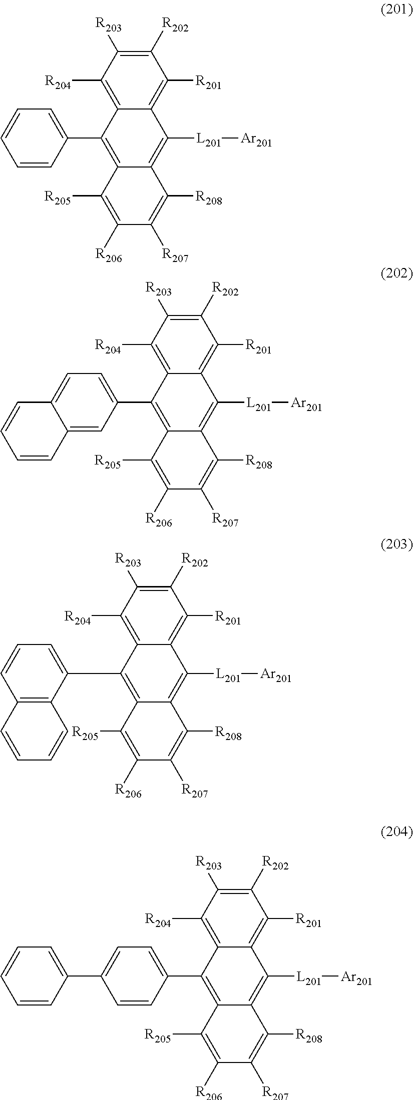

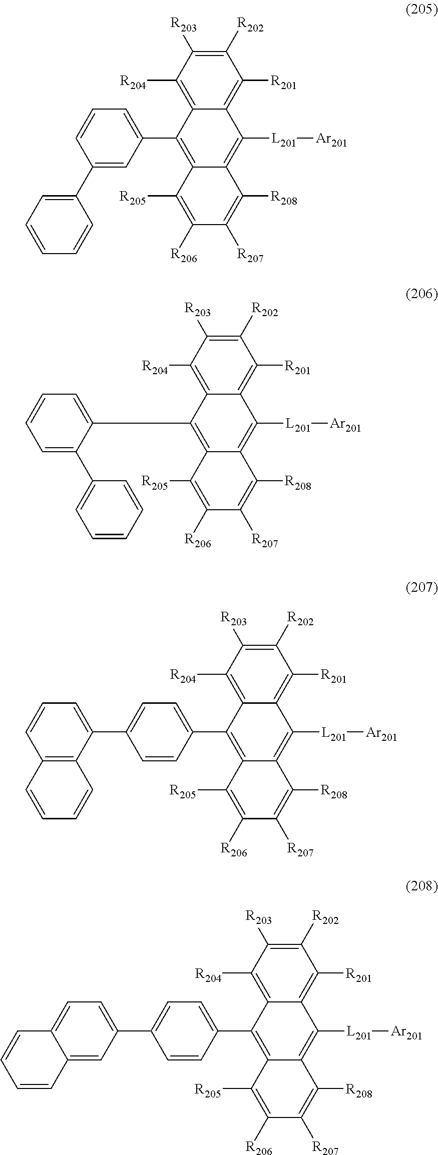

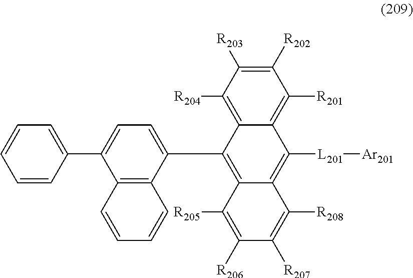

In the formulae (201) to (209):
L₂₀₁ and Ar₂₀₁ represent the same as L₂₀₁ and Ar₂₀₁ in the formula (2); and
R₂₀₁ to R₂₀₈ each independently represent the same as R₂₀₁ to R₂₀₈ in the formula (2).

The second compound represented by the formula (2) is also preferably a compound represented by a formula (221), a formula (222), a formula (223), a formula (224), a formula (225), a formula (226), a formula (227), a formula (228), or a formula (229) below.

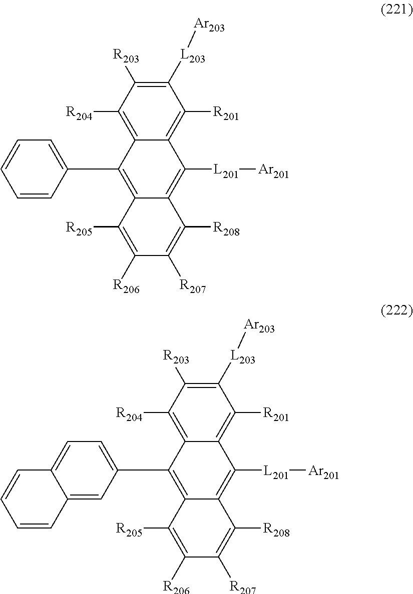

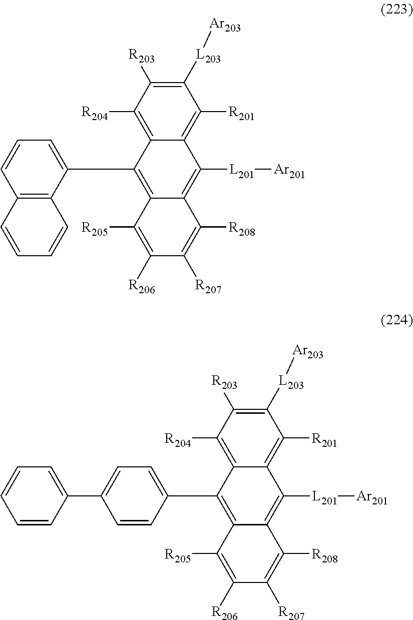

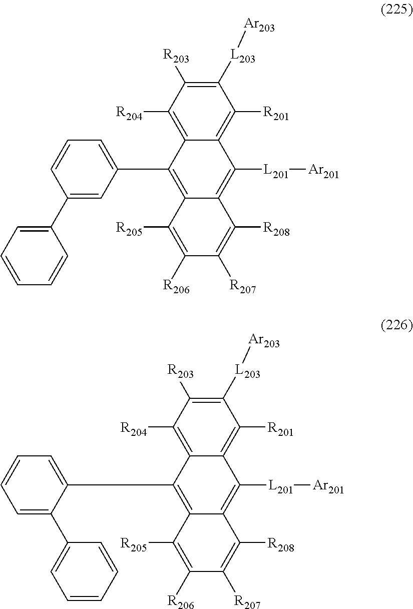

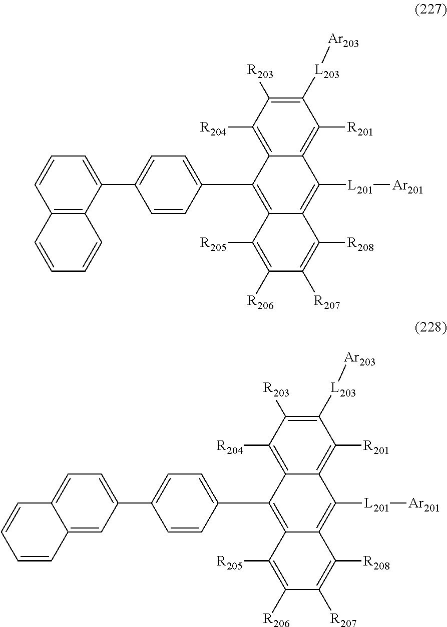

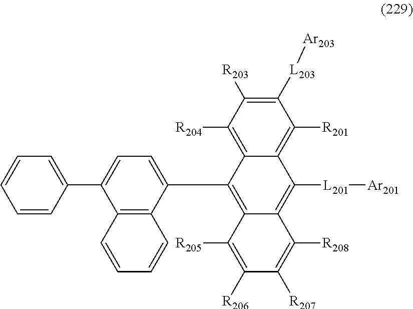

In the formulae (221), (222), (223), (224), (225), (226), (227), (228) and (229):
R₂₀₁ and R₂₀₃ to R₂₀₈ each independently represent the same as R₂₀₁ and R₂₀₃ to R₂₀₈ in the formula (2);
L₂₀₁ and Ar₂₀₁ respectively represent the same as L₂₀₁ and Ar₂₀₁ in the formula (2);
L₂₀₃ represents the same as L₂₀₁ in the formula (2);
L₂₀₃ and L₂₀₁ are mutually the same or different;
Ar₂₀₃ represents the same as Ar₂₀₁ in the formula (2); and
Ar₂₀₃ and Ar₂₀₁ are mutually the same or different.

The second compound represented by the formula (2) is also preferably a compound represented by a formula (241), a formula (242), a formula (243), a formula (244), a formula (245), a formula (246), a formula (247), a formula (248), or a formula (249) below.

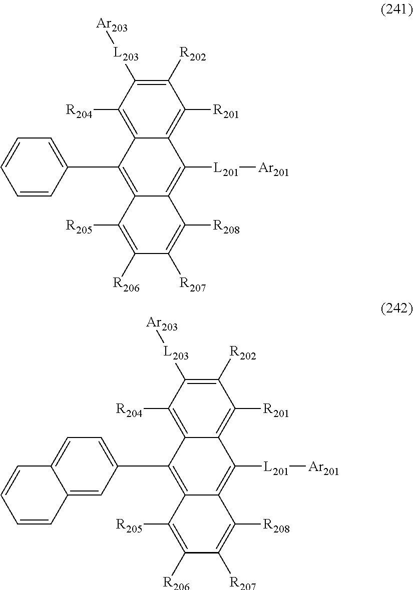

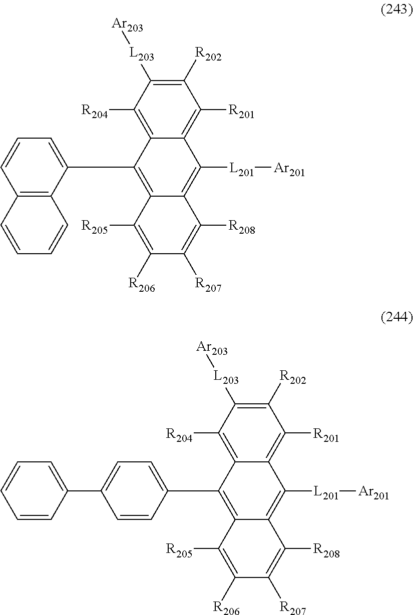

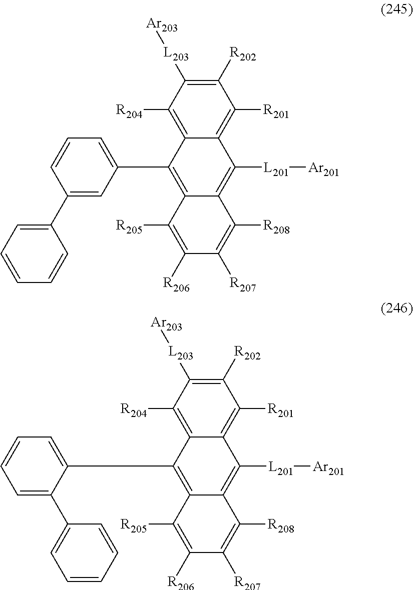

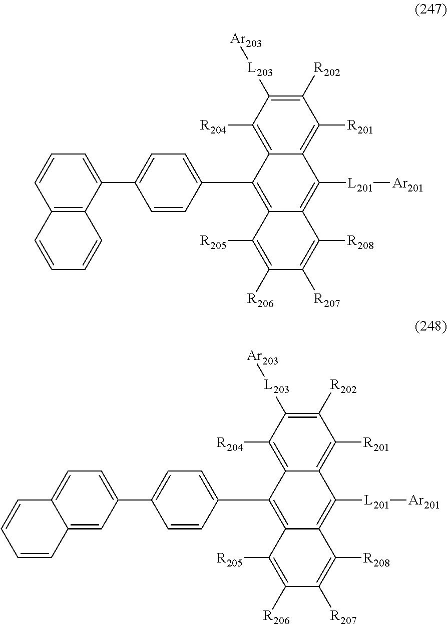

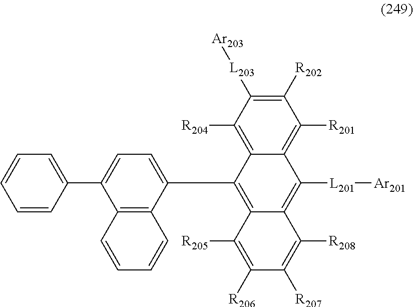

In the formulae (241), (242), (243), (244), (245), (246), (247), (248) and (249):
R₂₀₁, R₂₀₂ and R₂₀₄ to R₂₀₈ each independently represent the same as R₂₀₁, R₂₀₂ and R₂₀₄ to R₂₀₈ in the formula (2);
L₂₀₁ and Ar₂₀₁ respectively represent the same as L₂₀₁ and Ar₂₀₁ in the formula (2);
L₂₀₃ represents the same as L₂₀₁ in the formula (2);
L₂₀₃ and L₂₀₁ are mutually the same or different;
Ar₂₀₃ represents the same as Ar₂₀₁ in the formula (2); and
Ar₂₀₃ and Ar₂₀₁ are mutually the same or different.

In the second compound represented by the formula (2), R₂₀₁ to R₂₀₈ are preferably each independently a hydrogen atom, a substituted or unsubstituted alkyl group having 1 to 50 carbon atoms, a substituted or unsubstituted cycloalkyl group having 3 to 50 ring carbon atoms, or a group represented by —Si(R₉₀₁)(R₉₀₂)(R₉₀₃).

L₂₀₁ is preferably a single bond or an unsubstituted arylene group having 6 to 22 ring carbon atoms, and Ar₂₀₁ is preferably a substituted or unsubstituted aryl group having 6 to 22 ring carbon atoms.

In the organic EL device according to the third exemplary embodiment, R₂₀₁ to R₂₀₈, which are substituents on the anthracene skeleton in the second compound represented by the formula (2), are each preferably a hydrogen atom in order to prevent intermolecular interaction from being inhibited and inhibit a decrease in electron mobility, but R₂₀₁ to R₂₀₈ may each be a substituted or unsubstituted aryl group having 6 to 50 ring carbon atoms or a substituted or unsubstituted heterocyclic group having 5 to 50 ring atoms.

If R₂₀₁ to R₂₀₈ are bulky substituents such as alkyl groups and cycloalkyl groups, intermolecular interaction may be inhibited to decrease the electron mobility of the second host material relative to that of the first host material, so that the relationship μe(H2)>μe(H1) shown by the numerical formula (Numerical Formula 30) may be unsatisfied.

When the second compound is used in the second emitting layer, satisfying the relationship μe(H2)>μe(H1) can inhibit a decrease in the recombination ability between holes and electrons in the first emitting layer and a decrease in luminous efficiency.

It should be noted that substituents, namely, a haloalkyl group, alkenyl group, alkynyl group, group represented by —Si(R₉₀₁)(R₉₀₂)(R₉₀₃), group represented by —O—(R₉₀₄), group represented by —S—(R₉₀₅), group represented by —N(R₉₀₆)(R₉₀₇), aralkyl group, group represented by —C(═O)R₈₀₁, group represented by —COOR₈₀₂, halogen atom, cyano group, and nitro group are likely to be bulky, and an alkyl group and cycloalkyl group are likely to be further bulky.

In the second compound represented by the formula (2), R₂₀₁ to R₂₀₈, which are the substituents on the anthracene skeleton, are each preferably not a bulky substituent and preferably not an alkyl group and cycloalkyl group. More preferably, R₂₀₁ to R₂₀₈ are each not an alkyl group, cycloalkyl group, haloalkyl group, alkenyl group, alkynyl group, group represented by —Si(R₉₀₁)(R₉₀₂)(R₉₀₃), group represented by —O—(R₉₀₄), group represented by —S—(R₉₀₅), group represented by —N(R₉₀₆)(R₉₀₇), aralkyl group, group represented by —C(═O)R₈₀₁, group represented by —COOR₈₀₂, halogen atom, cyano group, and nitro group.

In the organic EL device according to the third exemplary embodiment, R₂₀₁ to R₂₀₈ in the second compound represented by the formula (2) are also preferably each independently a hydrogen atom, a substituted or unsubstituted alkyl group having 1 to 50 carbon atoms, a substituted or unsubstituted cycloalkyl group having 3 to 50 ring carbon atoms, or a group represented by —Si(R₉₀₁)(R₉₀₂)(R₉₀₃).

In the organic EL device according to the third exemplary embodiment, R₂₀₁ to R₂₀₈ in the second compound represented by the formula (2) are each preferably a hydrogen atom.

In the second compound, examples of the substituent for the “substituted or unsubstituted” group on R₂₀₁ to R₂₀₈ also preferably do not include the above-described substituent that is likely to be bulky, especially a substituted or unsubstituted alkyl group and a substituted or unsubstituted cycloalkyl group.

When examples of the substituent for the “substituted or unsubstituted” group on R₂₀₁ to R₂₀₈ do not include a substituted or unsubstituted alkyl group and a substituted or unsubstituted cycloalkyl group, inhibition of intermolecular interaction to be caused by presence of a bulky substituent such as an alkyl group and a cycloalkyl group can be prevented, thereby preventing a decrease in the electron mobility. Moreover, when the second compound described above is used in the second emitting layer, a decrease in a recombination ability between holes and electrons in the first emitting layer and a decrease in the luminous efficiency can be inhibited.

Further preferably, R₂₀₁ to R₂₀₈ that are the substituents on the anthracene skeleton are not bulky substituents and R₂₀₁ to R₂₀₈ as substituents are unsubstituted.

Assuming that R₂₀₁ to R₂₀₈ that are the substituents on the anthracene skeleton are not bulky substituents and substituents are bonded to R₂₀₁ to R₂₀₈ that are not bulky substituents, the substituents bonded to R₂₀₁ to R₂₀₈ are preferably not bulky substituents; and the substituents bonded to R₂₀₁ to R₂₀₈ serving as substituents are preferably not an alkyl group and cycloalkyl group, more preferably not an alkyl group, cycloalkyl group, haloalkyl group, alkenyl group, alkynyl group, group represented by —Si(R₉₀₁)(R₉₀₂)(R₉₀₃), group represented by —O—(R₉₀₄), group represented by —S—(R₉₀₅), group represented by —N(R₉₀₆)(R₉₀₇), aralkyl group, group represented by —C(═O)R₈₀₁, group represented by —COOR₈₀₂, halogen atom, cyano group, and nitro group.

In the second compound, the groups specified to be “substituted or unsubstituted” are each preferably an “unsubstituted” group.

### Method of Producing Second Compound

The second compound can be produced by a known method.

The second compound can also be produced based on a known method through a known alternative reaction using a known material(s) tailored for the target compound.

### Specific Examples of Second Compound

Specific examples of the second compound include the following compounds.

It should however be noted that the invention is not limited to the specific examples of the second compound.

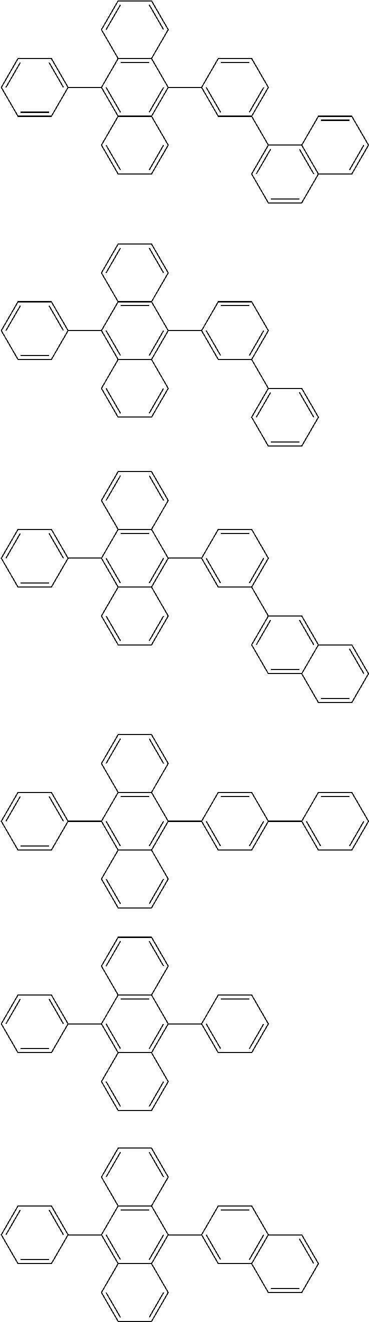

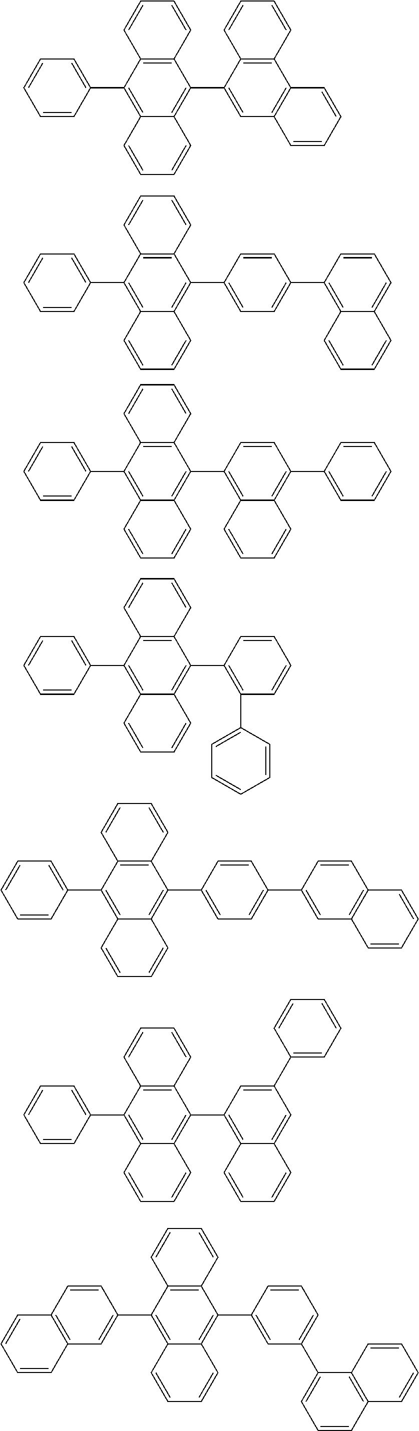

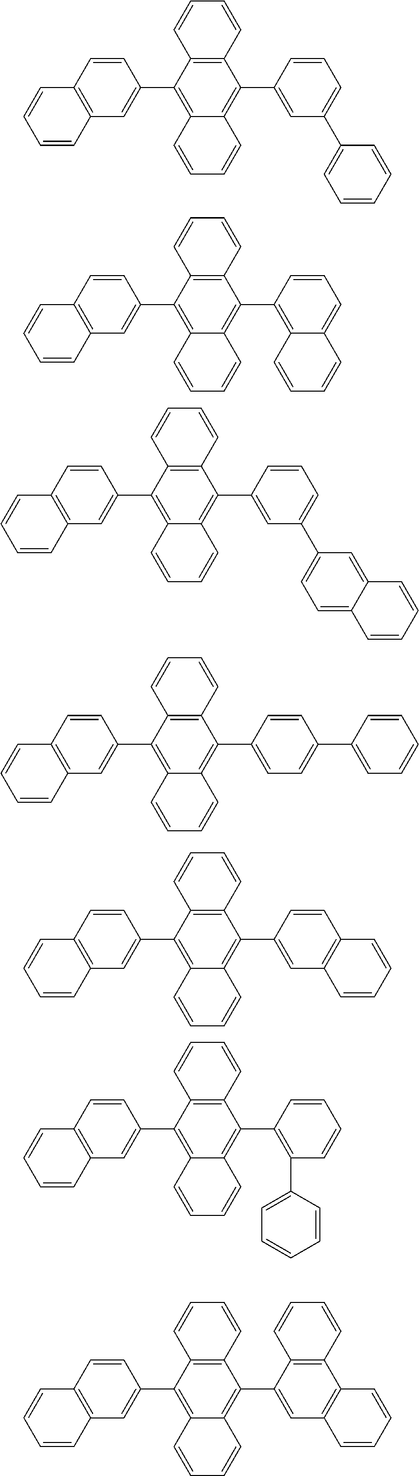

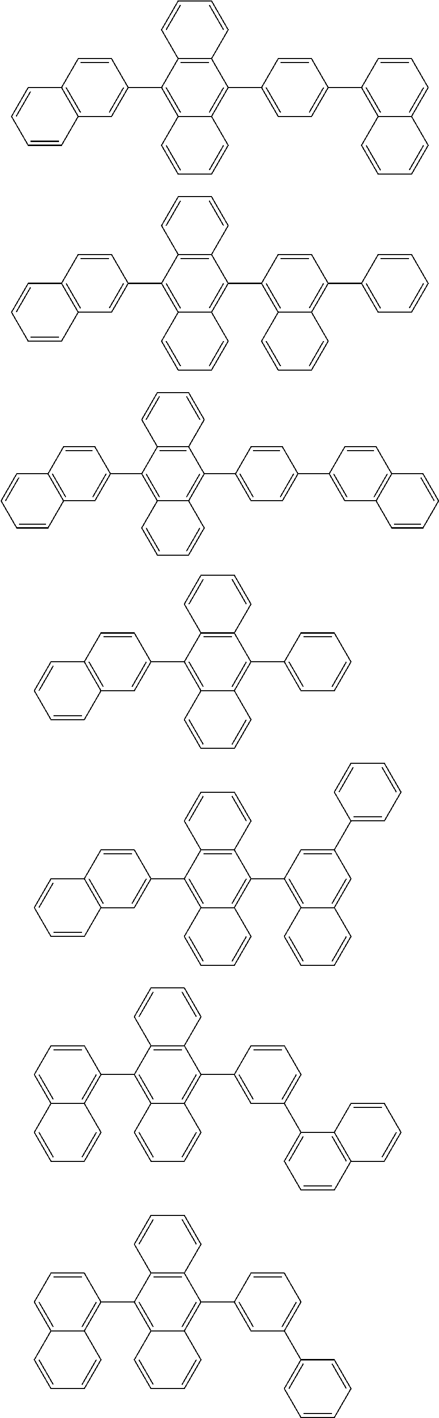

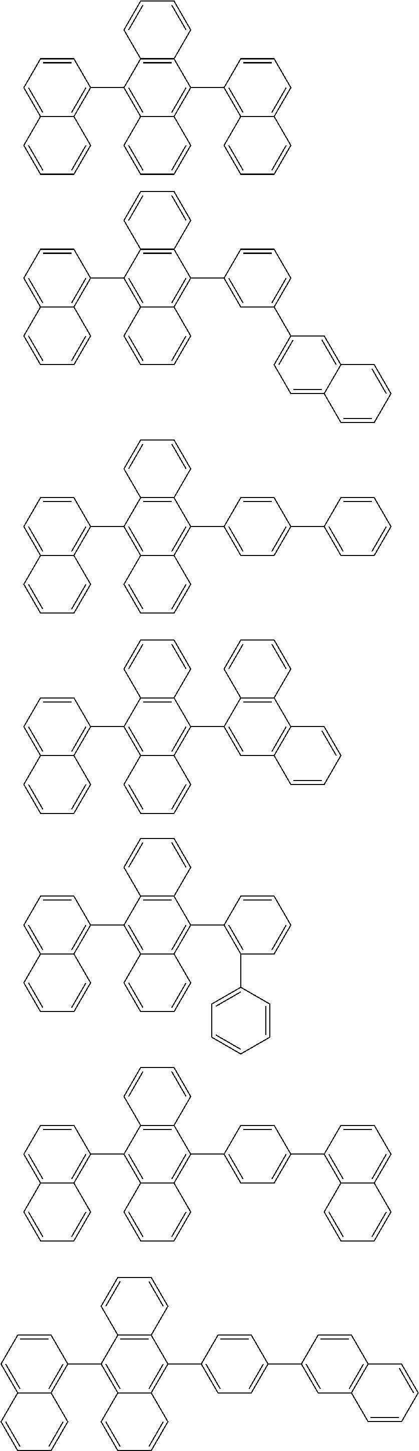

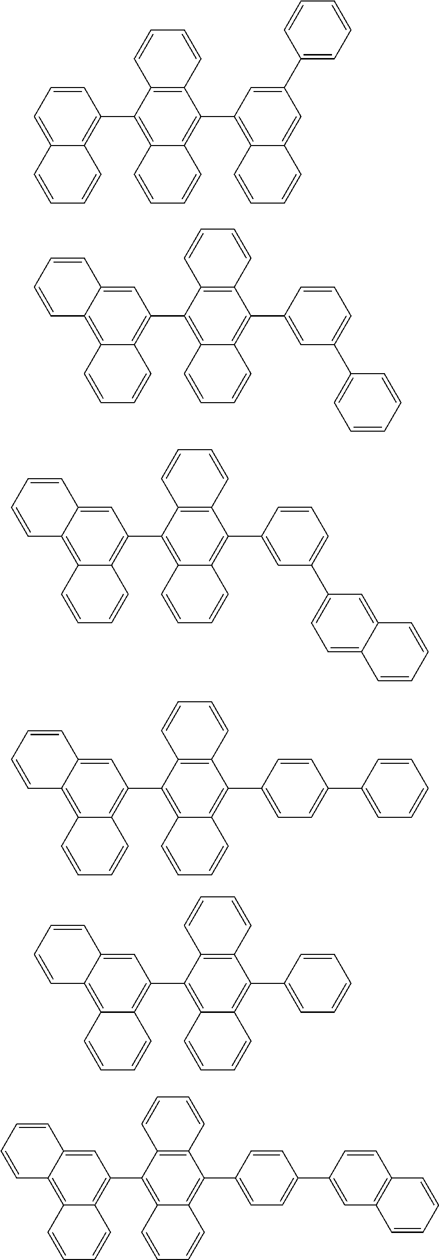

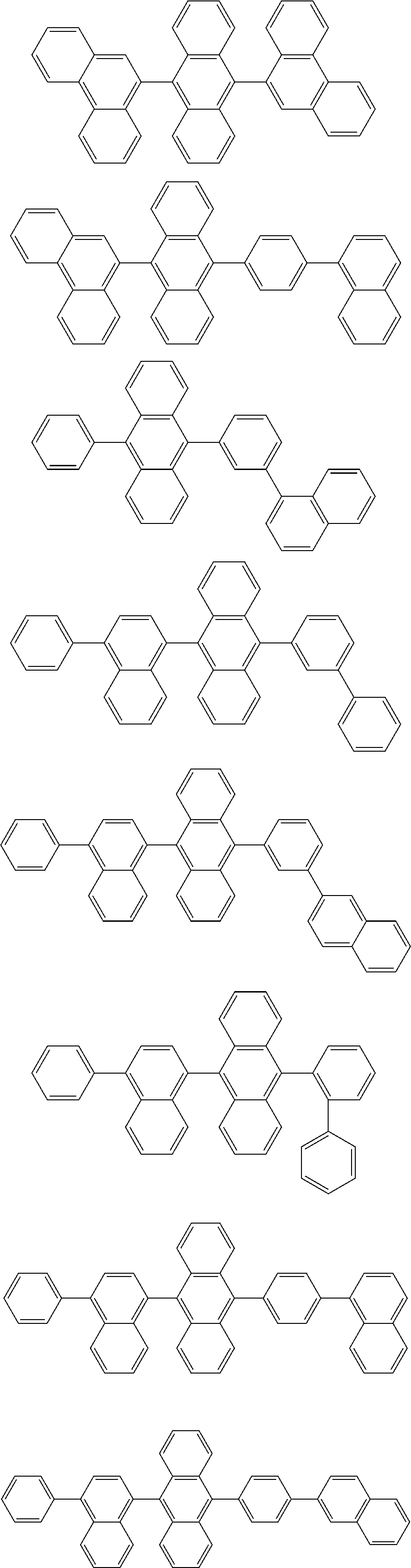

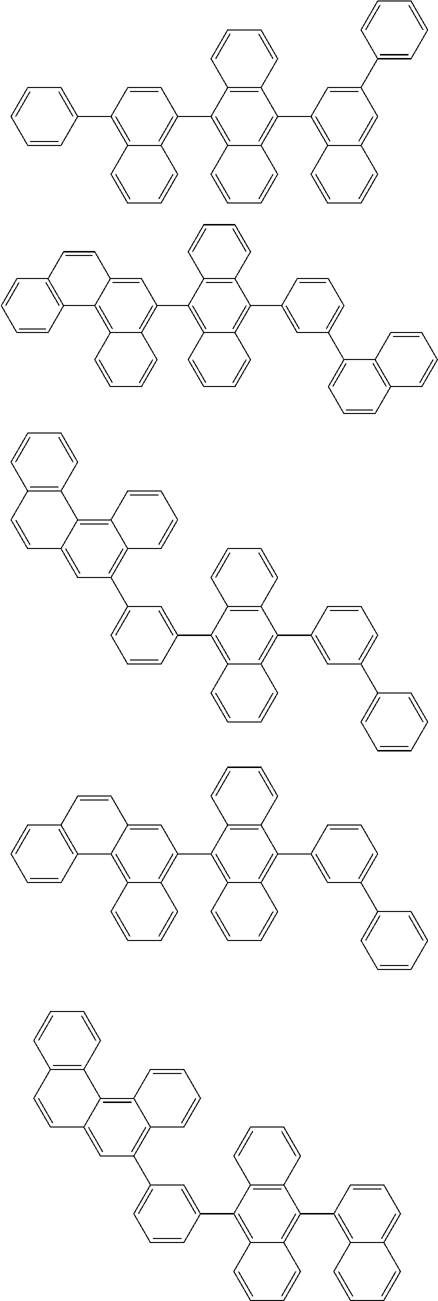

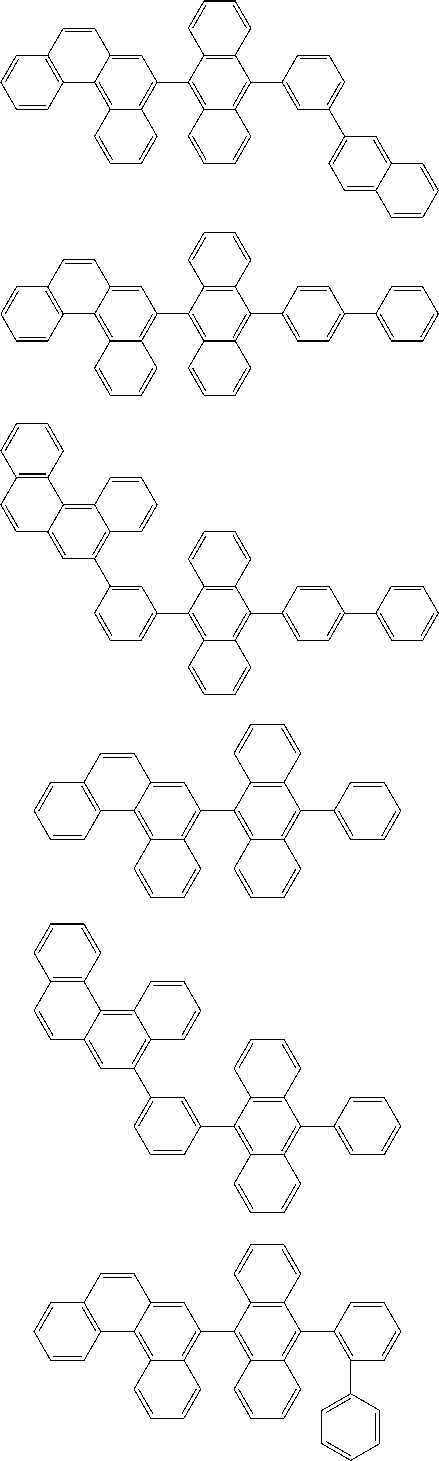

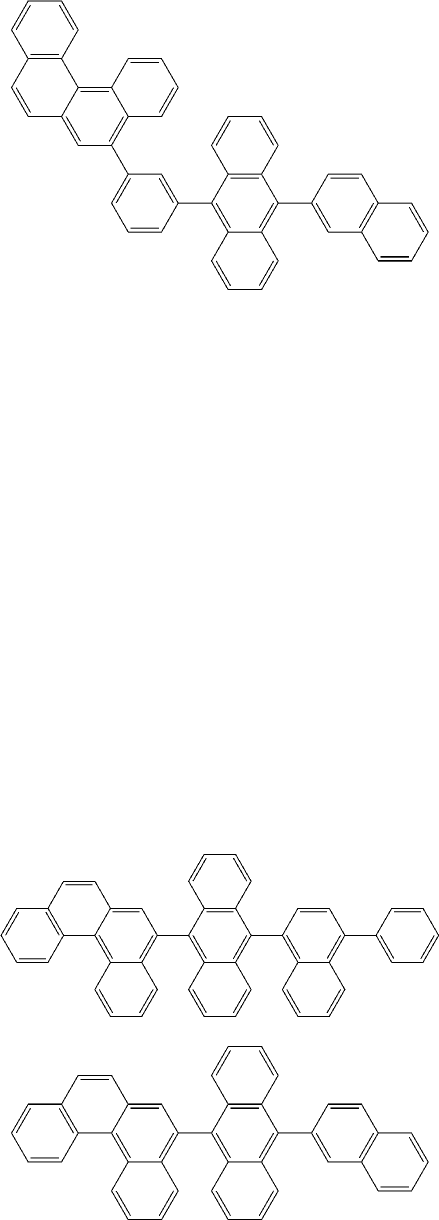

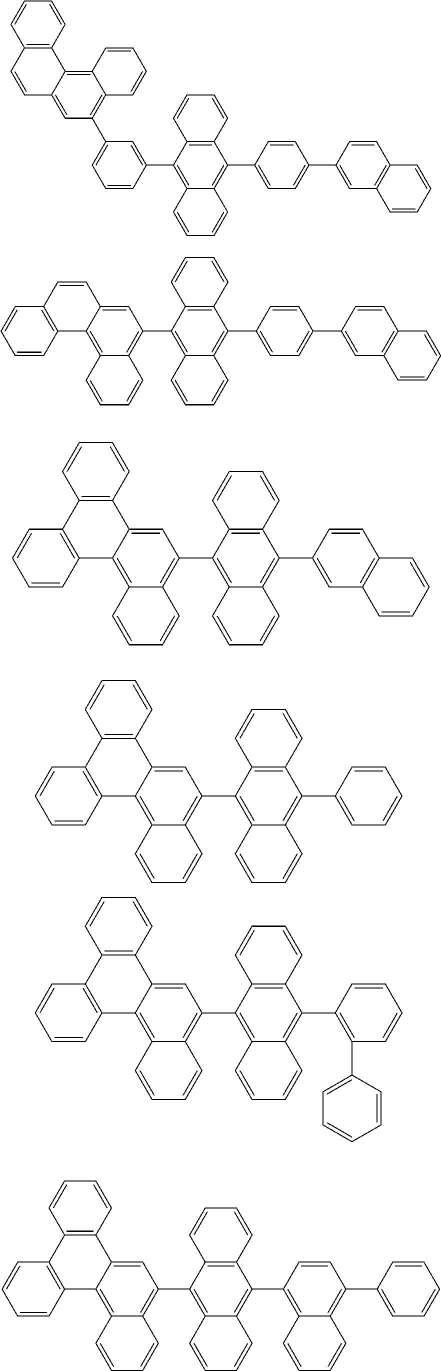

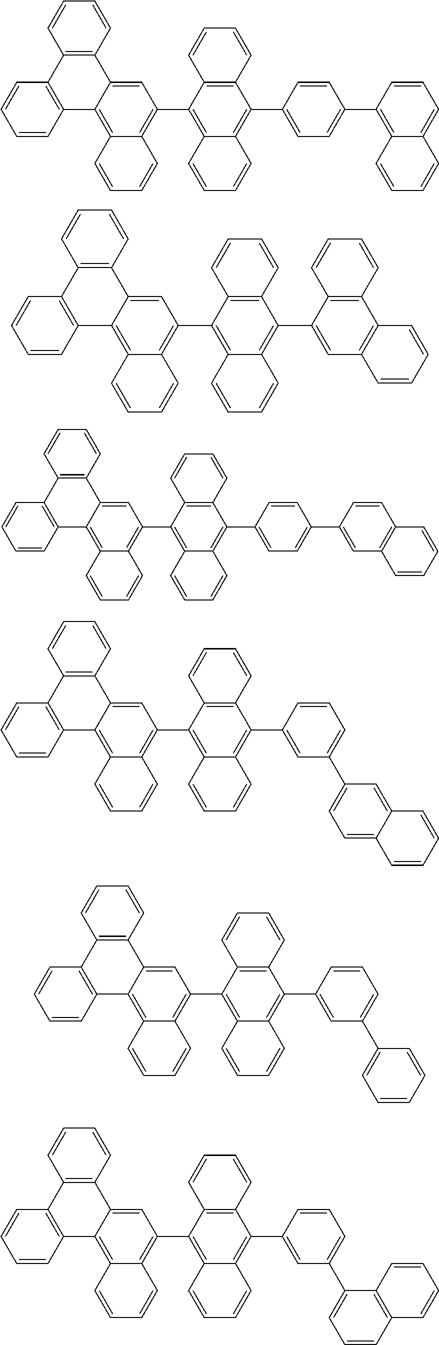

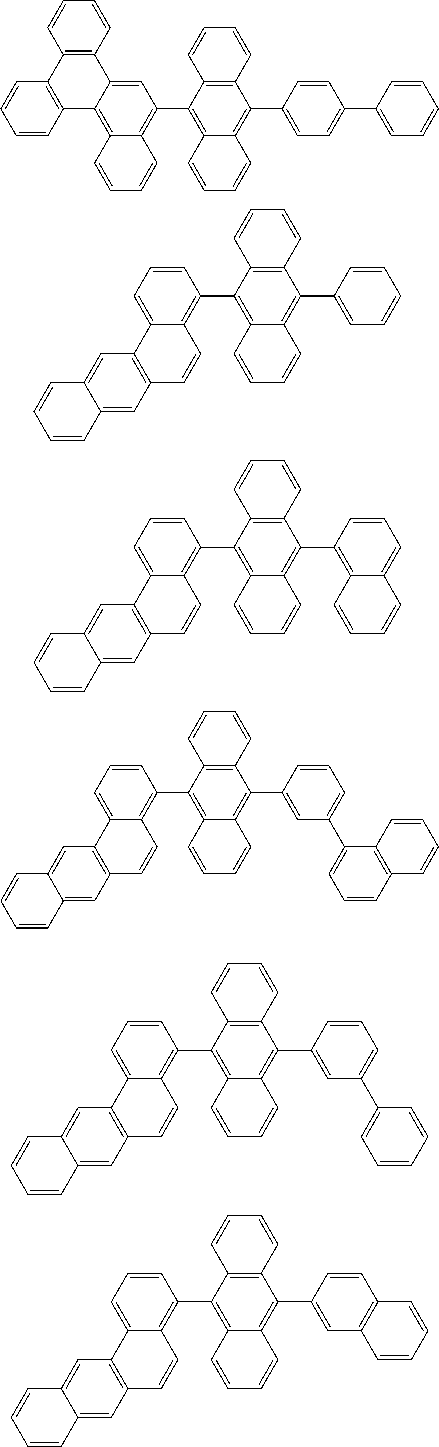

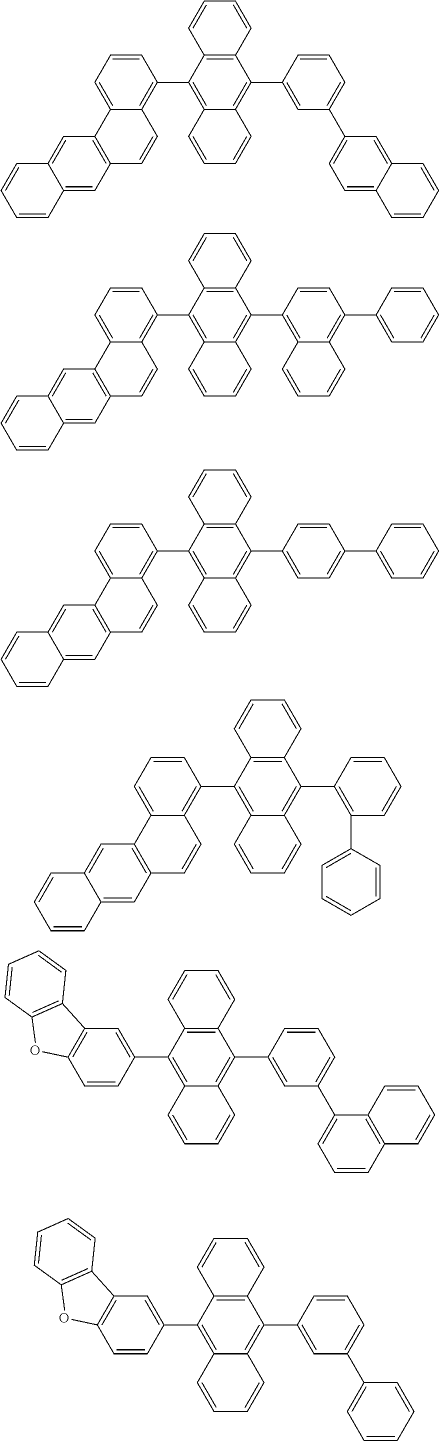

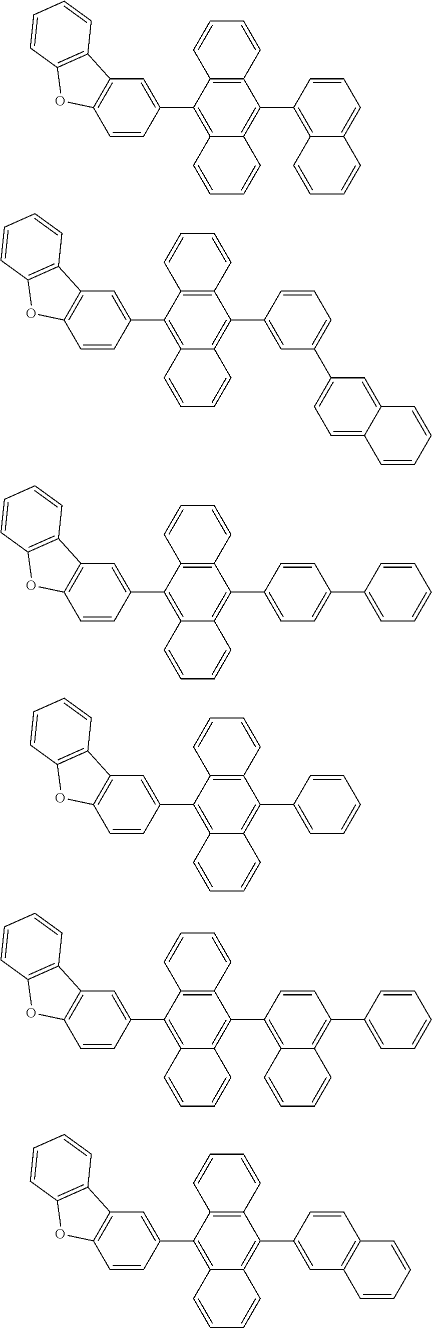

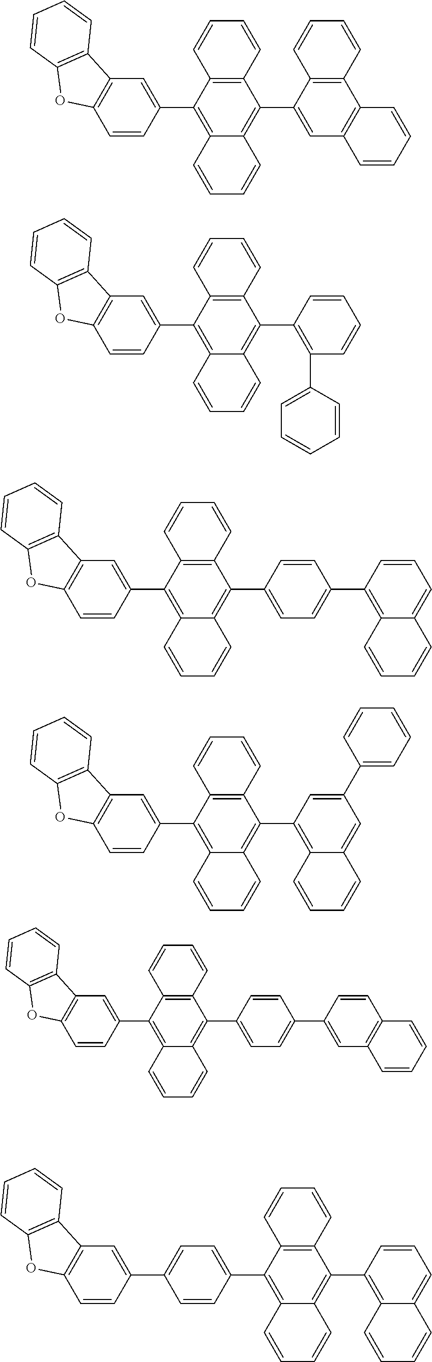

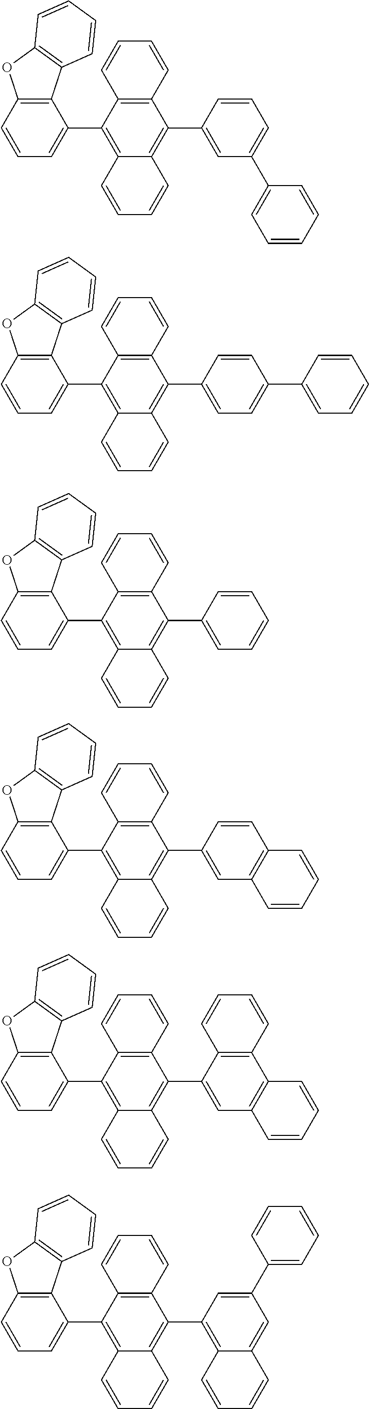

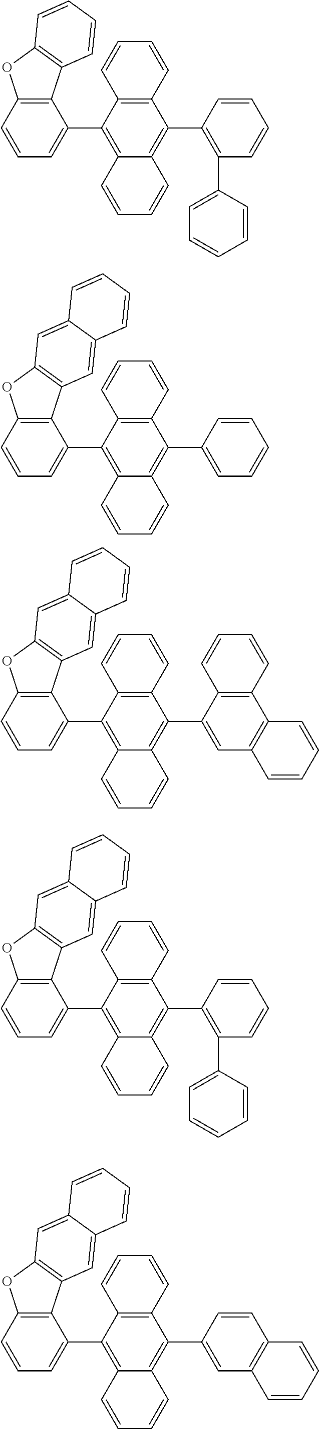

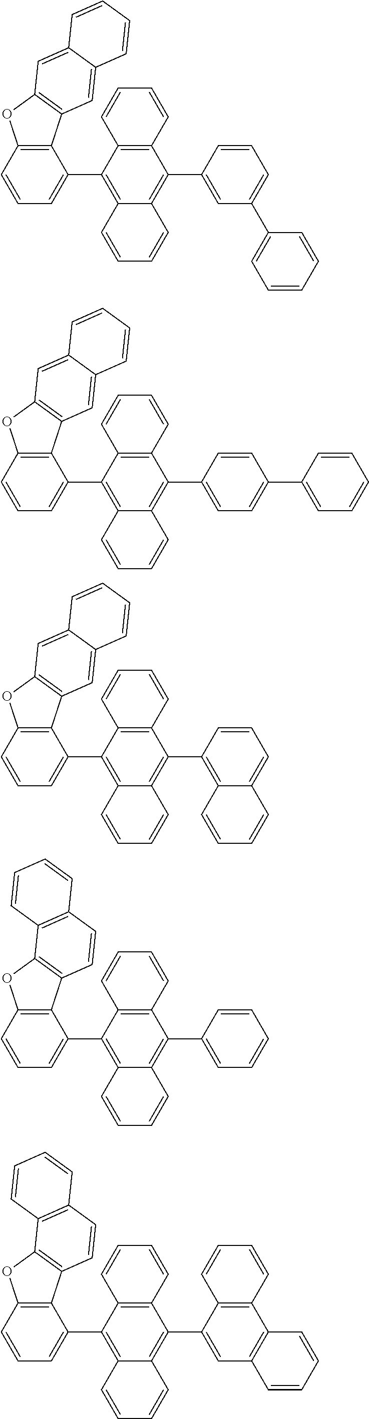

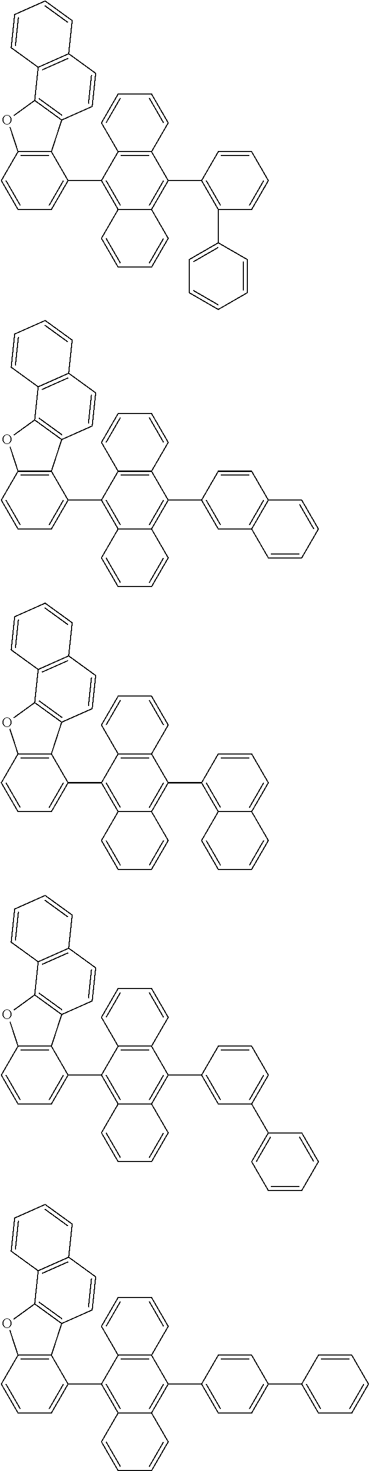 [SMILES: c1ccc(-c2ccccc2-c2c3ccccc3c(-c3cccc4oc5c6ccccc6ccc5c34)c3ccccc23)cc1 ; c1ccc2cc(-c3c4ccccc4c(-c4cccc5oc6c7ccccc7ccc6c45)c4ccccc34)ccc2c1 ; c1ccc2c(-c3c4ccccc4c(-c4cccc5oc6c7ccccc7ccc6c45)c4ccccc34)cccc2c1 ; c1ccc(-c2cccc(-c3c4ccccc4c(-c4cccc5oc6c7ccccc7ccc6c45)c4ccccc34)c2)cc1 ; c1ccc(-c2ccc(-c3c4ccccc4c(-c4cccc5oc6c7ccccc7ccc6c45)c4ccccc34)cc2)cc1]

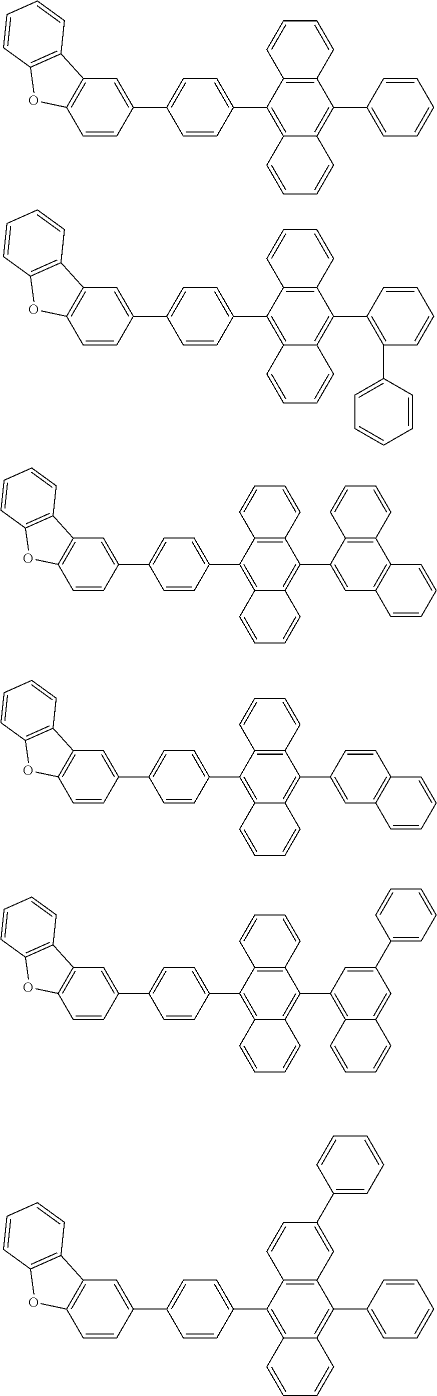

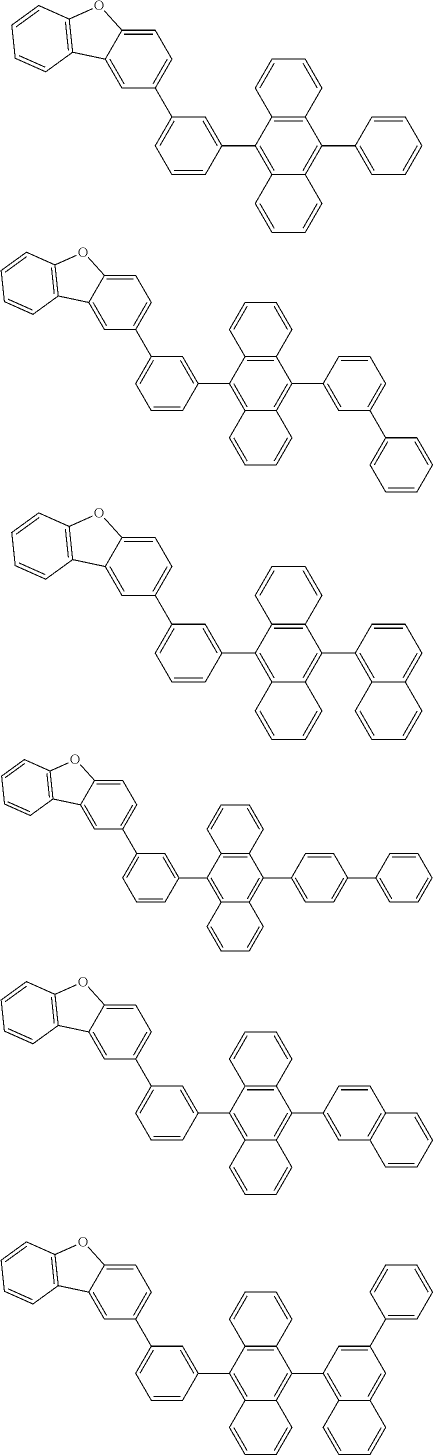 [SMILES: c1ccc(-c2c3ccccc3c(-c3cccc(-c4ccc5oc6ccccc6c5c4)c3)c3ccccc23)cc1 ; c1ccc(-c2cccc(-c3c4ccccc4c(-c4cccc(-c5ccc6oc7ccccc7c6c5)c4)c4ccccc34)c2)cc1 ; c1cc(-c2ccc3oc4ccccc4c3c2)cc(-c2c3ccccc3c(-c3cccc4ccccc34)c3ccccc23)c1 ; c1ccc(-c2ccc(-c3c4ccccc4c(-c4cccc(-c5ccc6oc7ccccc7c6c5)c4)c4ccccc34)cc2)cc1 ; c1cc(-c2ccc3oc4ccccc4c3c2)cc(-c2c3ccccc3c(-c3ccc4ccccc4c3)c3ccccc23)c1 ; c1ccc(-c2cc(-c3c4ccccc4c(-c4cccc(-c5ccc6oc7ccccc7c6c5)c4)c4ccccc34)c3ccccc3c2)cc1]

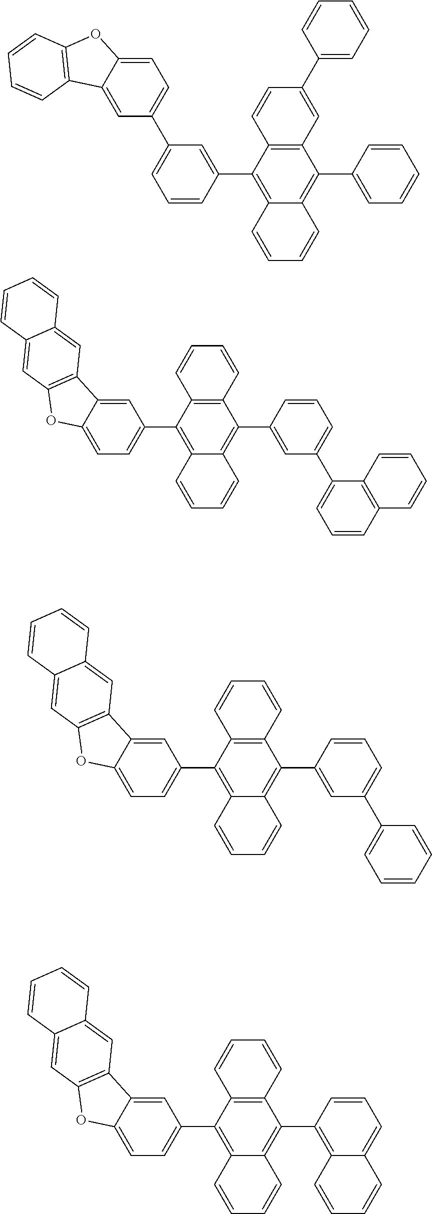 [SMILES: c1ccc(-c2ccc3c(-c4cccc(-c5ccc6oc7ccccc7c6c5)c4)c4ccccc4c(-c4ccccc4)c3c2)cc1 ; c1cc(-c2cccc3ccccc23)cc(-c2c3ccccc3c(-c3ccc4oc5cc6ccccc6cc5c4c3)c3ccccc23)c1 ; c1ccc(-c2cccc(-c3c4ccccc4c(-c4ccc5oc6cc7ccccc7cc6c5c4)c4ccccc34)c2)cc1 ; c1ccc2cc3c(cc2c1)oc1ccc(-c2c4ccccc4c(-c4cccc5ccccc45)c4ccccc24)cc13]

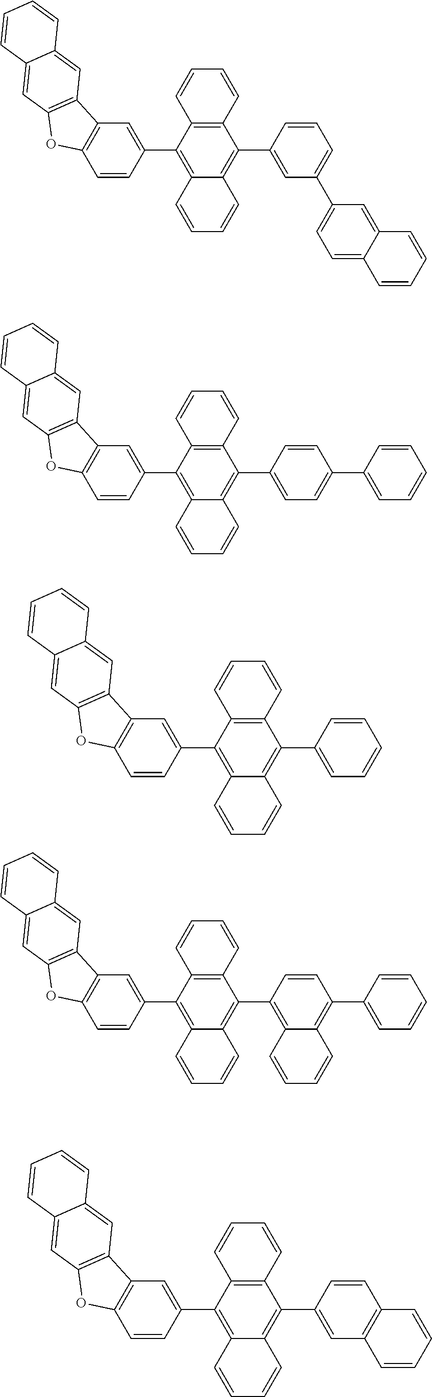 [SMILES: c1cc(-c2ccc3ccccc3c2)cc(-c2c3ccccc3c(-c3ccc4oc5cc6ccccc6cc5c4c3)c3ccccc23)c1 ; c1ccc(-c2ccc(-c3c4ccccc4c(-c4ccc5oc6cc7ccccc7cc6c5c4)c4ccccc34)cc2)cc1 ; c1ccc(-c2c3ccccc3c(-c3ccc4oc5cc6ccccc6cc5c4c3)c3ccccc23)cc1 ; c1ccc(-c2ccc(-c3c4ccccc4c(-c4ccc5oc6cc7ccccc7cc6c5c4)c4ccccc34)c3ccccc23)cc1 ; c1ccc2cc(-c3c4ccccc4c(-c4ccc5oc6cc7ccccc7cc6c5c4)c4ccccc34)ccc2c1]

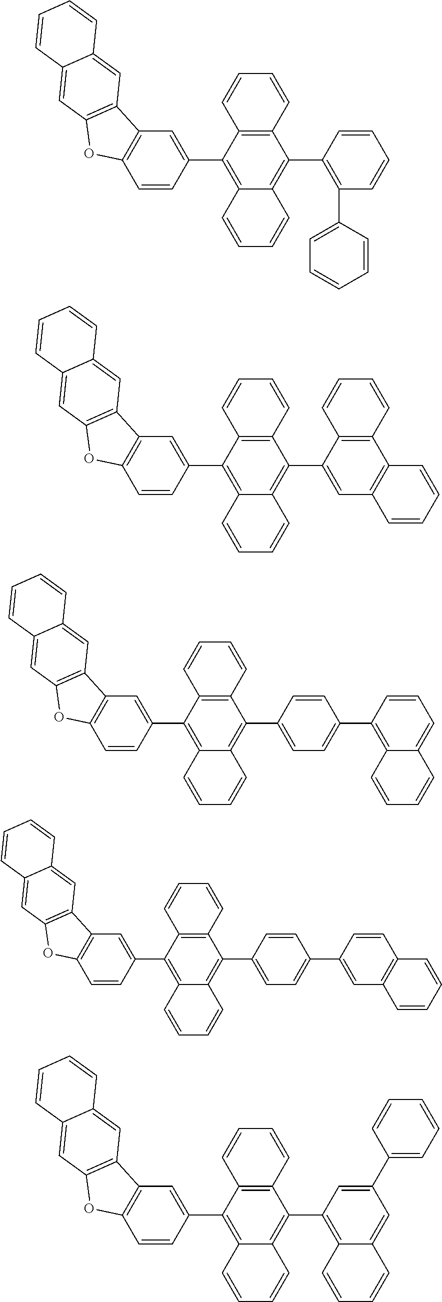 [SMILES: c1ccc(-c2ccccc2-c2c3ccccc3c(-c3ccc4oc5cc6ccccc6cc5c4c3)c3ccccc23)cc1 ; c1ccc2cc3c(cc2c1)oc1ccc(-c2c4ccccc4c(-c4cc5ccccc5c5ccccc45)c4ccccc24)cc13 ; c1ccc2cc3c(cc2c1)oc1ccc(-c2c4ccccc4c(-c4ccc(-c5cccc6ccccc56)cc4)c4ccccc24)cc13 ; c1ccc2cc(-c3ccc(-c4c5ccccc5c(-c5ccc6oc7cc8ccccc8cc7c6c5)c5ccccc45)cc3)ccc2c1 ; c1ccc(-c2cc(-c3c4ccccc4c(-c4ccc5oc6cc7ccccc7cc6c5c4)c4ccccc34)c3ccccc3c2)cc1]

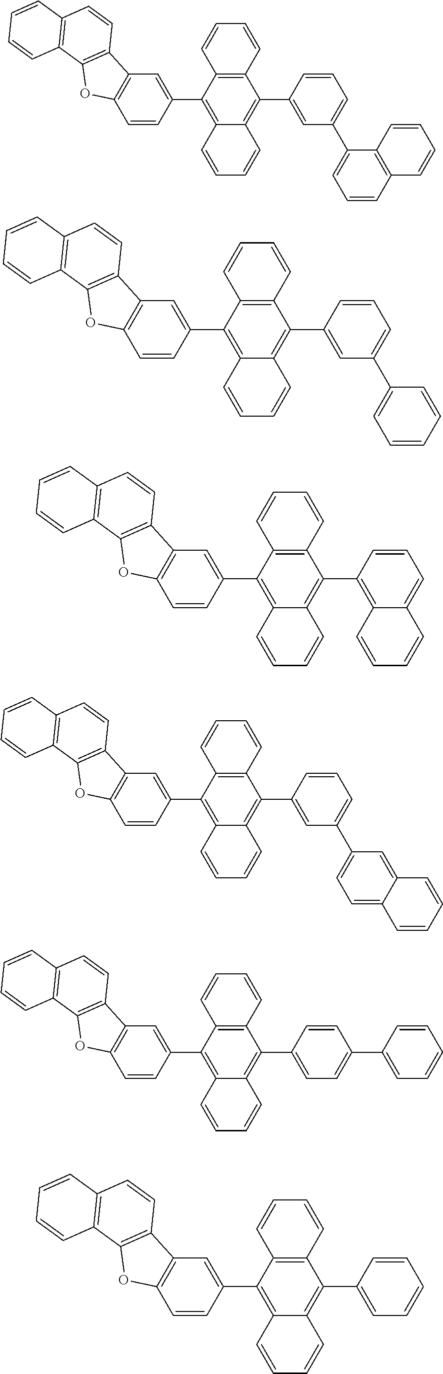 [SMILES: c1cc(-c2cccc3ccccc23)cc(-c2c3ccccc3c(-c3ccc4oc5c6ccccc6ccc5c4c3)c3ccccc23)c1 ; c1ccc(-c2cccc(-c3c4ccccc4c(-c4ccc5oc6c7ccccc7ccc6c5c4)c4ccccc34)c2)cc1 ; c1ccc2c(-c3c4ccccc4c(-c4ccc5oc6c7ccccc7ccc6c5c4)c4ccccc34)cccc2c1 ; c1cc(-c2ccc3ccccc3c2)cc(-c2c3ccccc3c(-c3ccc4oc5c6ccccc6ccc5c4c3)c3ccccc23)c1 ; c1ccc(-c2ccc(-c3c4ccccc4c(-c4ccc5oc6c7ccccc7ccc6c5c4)c4ccccc34)cc2)cc1 ; c1ccc(-c2c3ccccc3c(-c3ccc4oc5c6ccccc6ccc5c4c3)c3ccccc23)cc1]

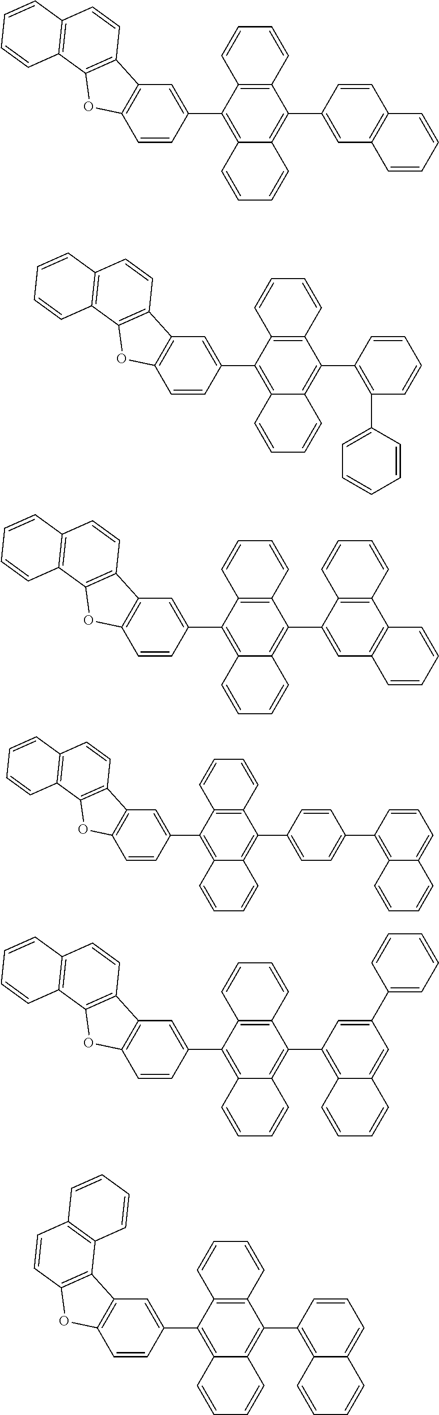

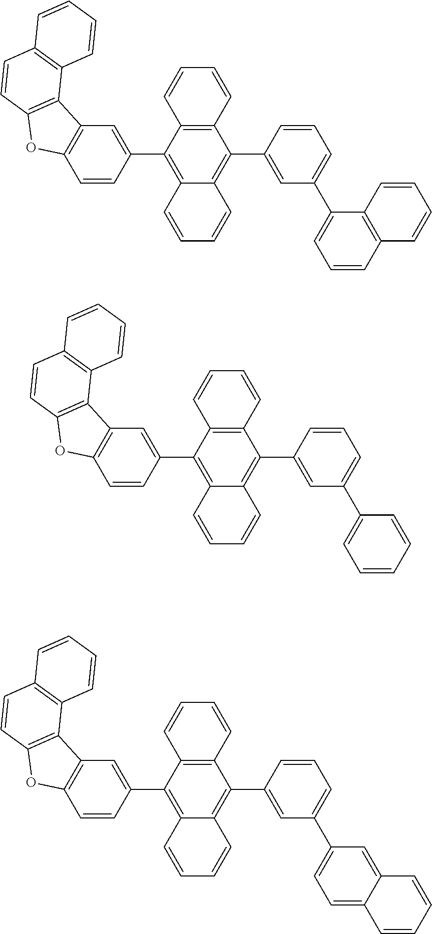 [SMILES: c1cc(-c2cccc3ccccc23)cc(-c2c3ccccc3c(-c3ccc4oc5ccc6ccccc6c5c4c3)c3ccccc23)c1 ; c1ccc(-c2cccc(-c3c4ccccc4c(-c4ccc5oc6ccc7ccccc7c6c5c4)c4ccccc34)c2)cc1 ; c1cc(-c2ccc3ccccc3c2)cc(-c2c3ccccc3c(-c3ccc4oc5ccc6ccccc6c5c4c3)c3ccccc23)c1]

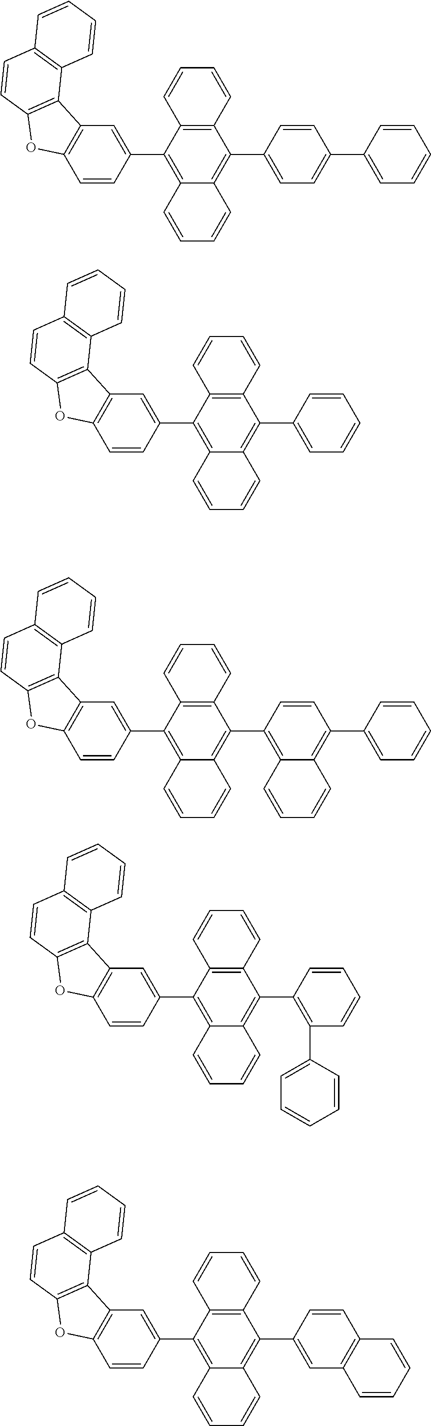 [SMILES: c1ccc(-c2ccc(-c3c4ccccc4c(-c4ccc5oc6ccc7ccccc7c6c5c4)c4ccccc34)cc2)cc1 ; c1ccc(-c2c3ccccc3c(-c3ccc4oc5ccc6ccccc6c5c4c3)c3ccccc23)cc1 ; c1ccc(-c2ccc(-c3c4ccccc4c(-c4ccc5oc6ccc7ccccc7c6c5c4)c4ccccc34)c3ccccc23)cc1 ; c1ccc(-c2ccccc2-c2c3ccccc3c(-c3ccc4oc5ccc6ccccc6c5c4c3)c3ccccc23)cc1 ; c1ccc2cc(-c3c4ccccc4c(-c4ccc5oc6ccc7ccccc7c6c5c4)c4ccccc34)ccc2c1]

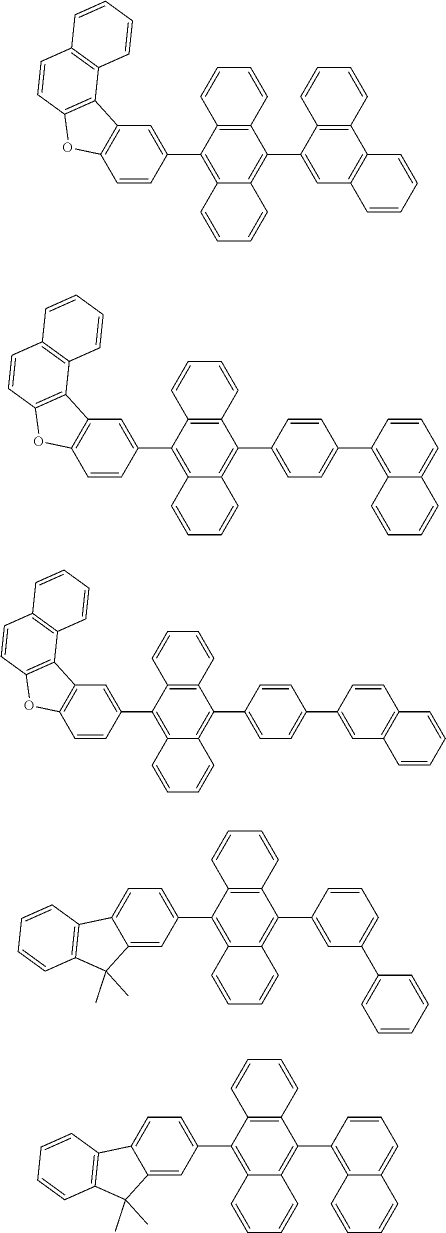

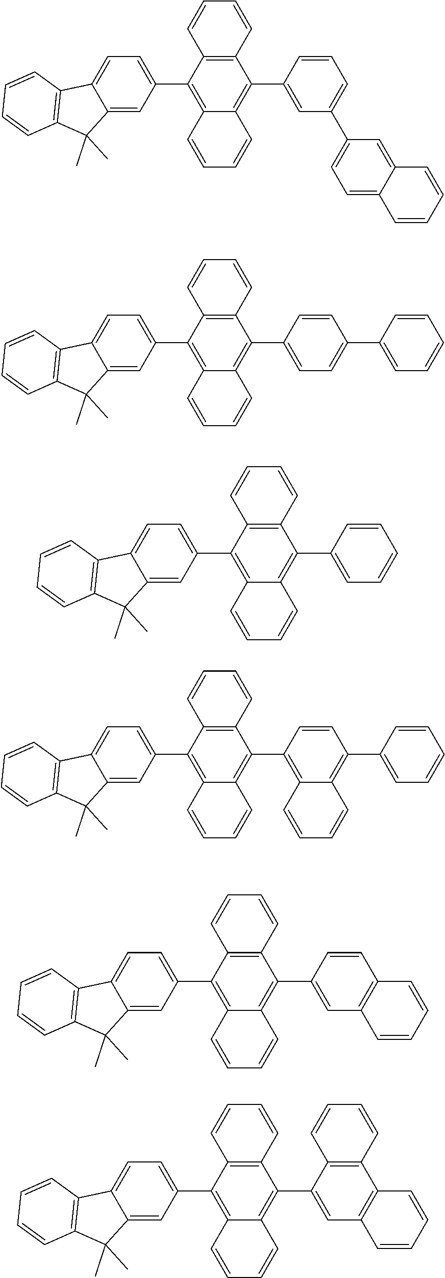

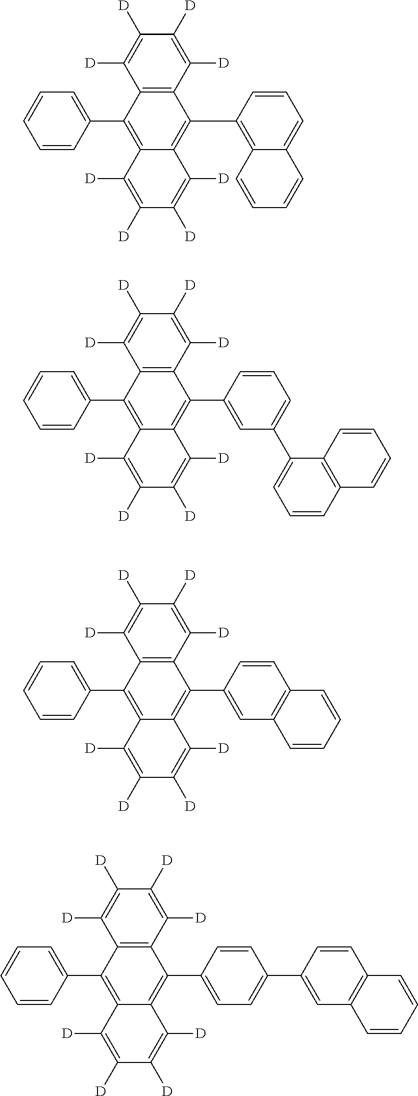 [SMILES: [2H]c1c([2H])c([2H])c2c(-c3cccc4ccccc34)c3c([2H])c([2H])c([2H])c([2H])c3c(-c3ccccc3)c2c1[2H] ; [2H]c1c([2H])c([2H])c2c(-c3cccc(-c4cccc5ccccc45)c3)c3c([2H])c([2H])c([2H])c([2H])c3c(-c3ccccc3)c2c1[2H] ; [2H]c1c([2H])c([2H])c2c(-c3ccc4ccccc4c3)c3c([2H])c([2H])c([2H])c([2H])c3c(-c3ccccc3)c2c1[2H] ; [2H]c1c([2H])c([2H])c2c(-c3ccc(-c4ccc5ccccc5c4)cc3)c3c([2H])c([2H])c([2H])c([2H])c3c(-c3ccccc3)c2c1[2H]]

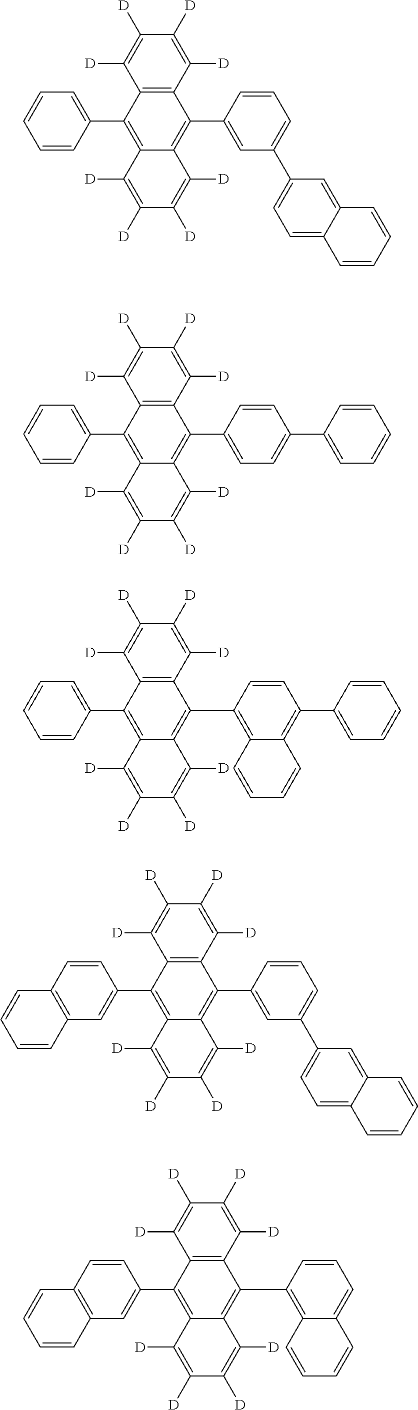

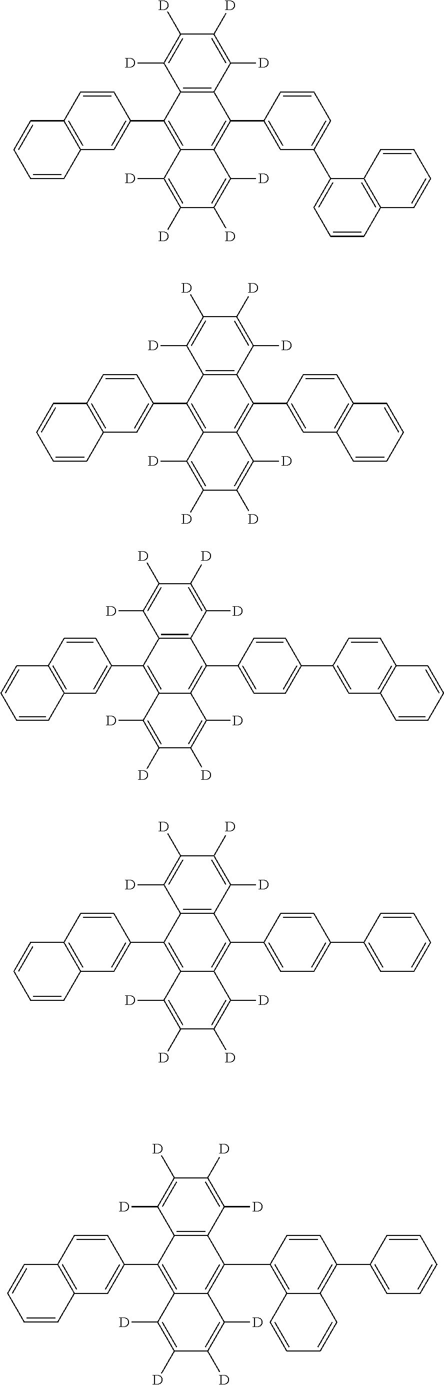

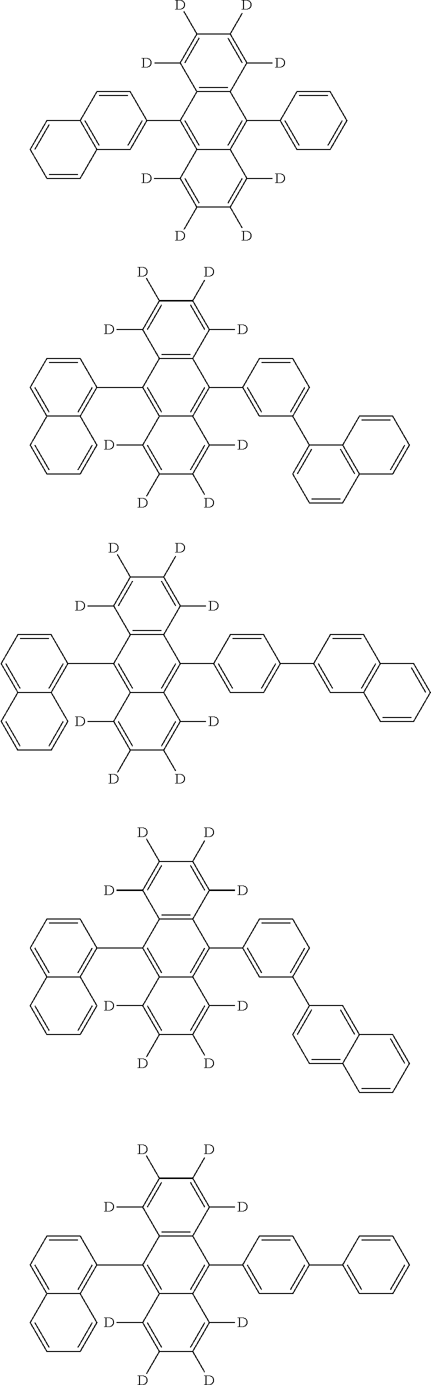

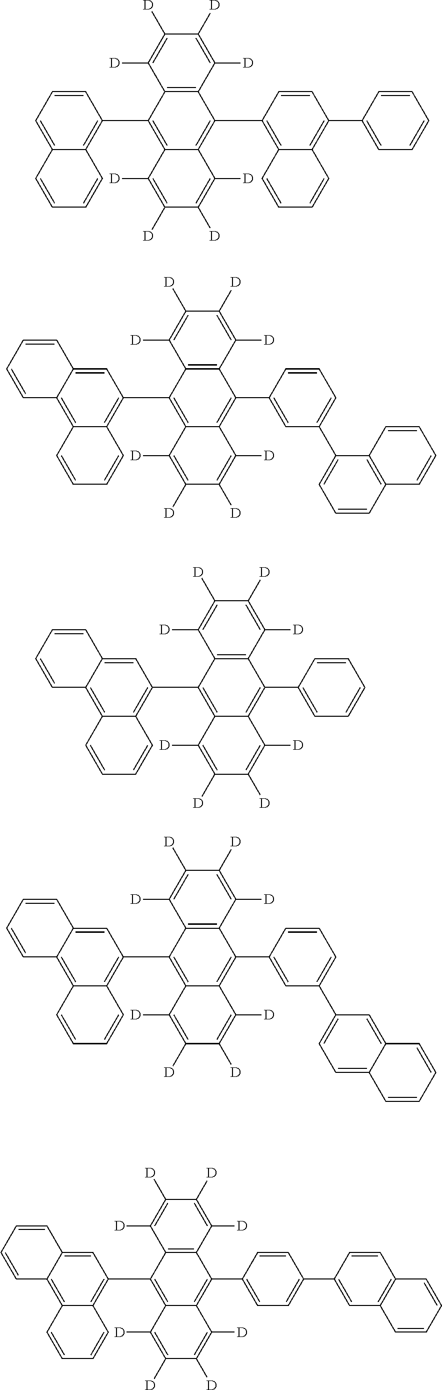

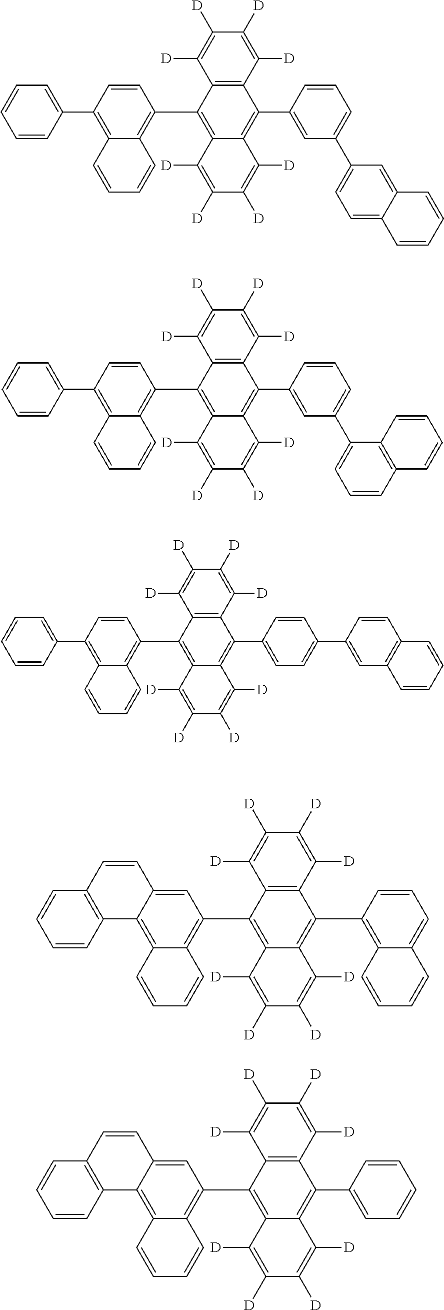

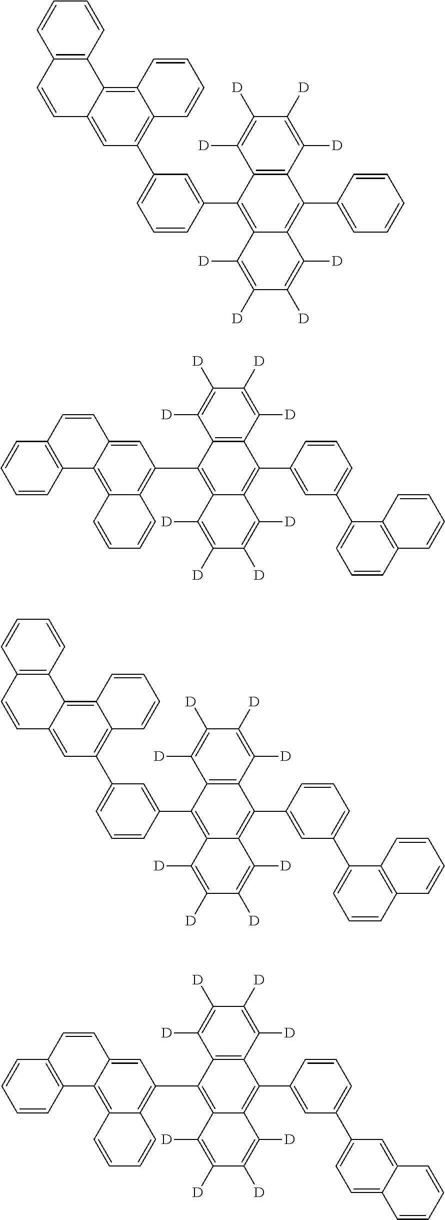 [SMILES: [2H]c1c([2H])c([2H])c2c(-c3cccc(-c4cc5ccc6ccccc6c5c5ccccc45)c3)c3c([2H])c([2H])c([2H])c([2H])c3c(-c3ccccc3)c2c1[2H] ; [2H]c1c([2H])c([2H])c2c(-c3cc4ccc5ccccc5c4c4ccccc34)c3c([2H])c([2H])c([2H])c([2H])c3c(-c3cccc(-c4cccc5ccccc45)c3)c2c1[2H] ; [2H]c1c([2H])c([2H])c2c(-c3cccc(-c4cc5ccc6ccccc6c5c5ccccc45)c3)c3c([2H])c([2H])c([2H])c([2H])c3c(-c3cccc(-c4cccc5ccccc45)c3)c2c1[2H] ; [2H]c1c([2H])c([2H])c2c(-c3cc4ccc5ccccc5c4c4ccccc34)c3c([2H])c([2H])c([2H])c([2H])c3c(-c3cccc(-c4ccc5ccccc5c4)c3)c2c1[2H]]

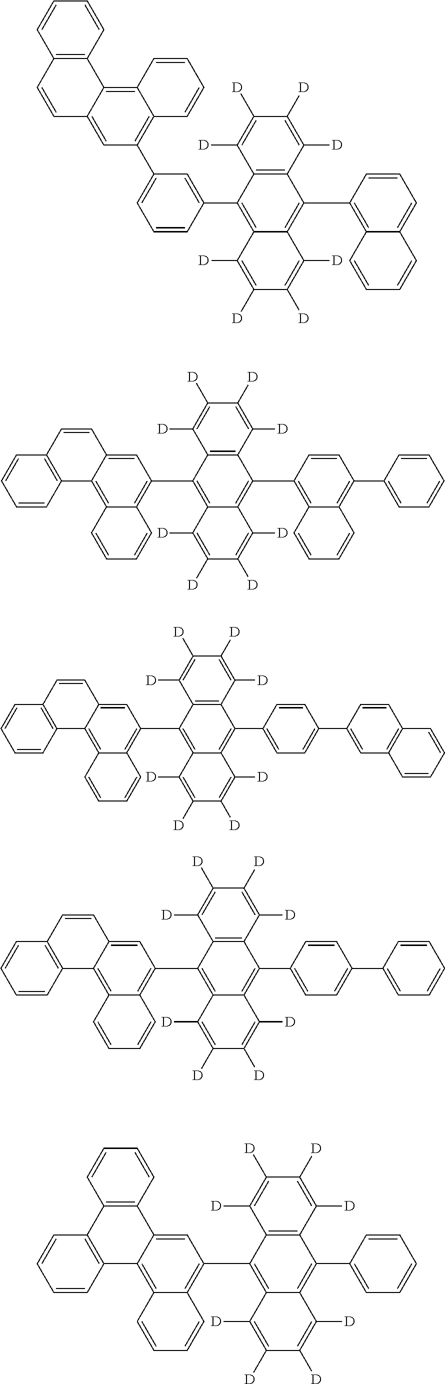

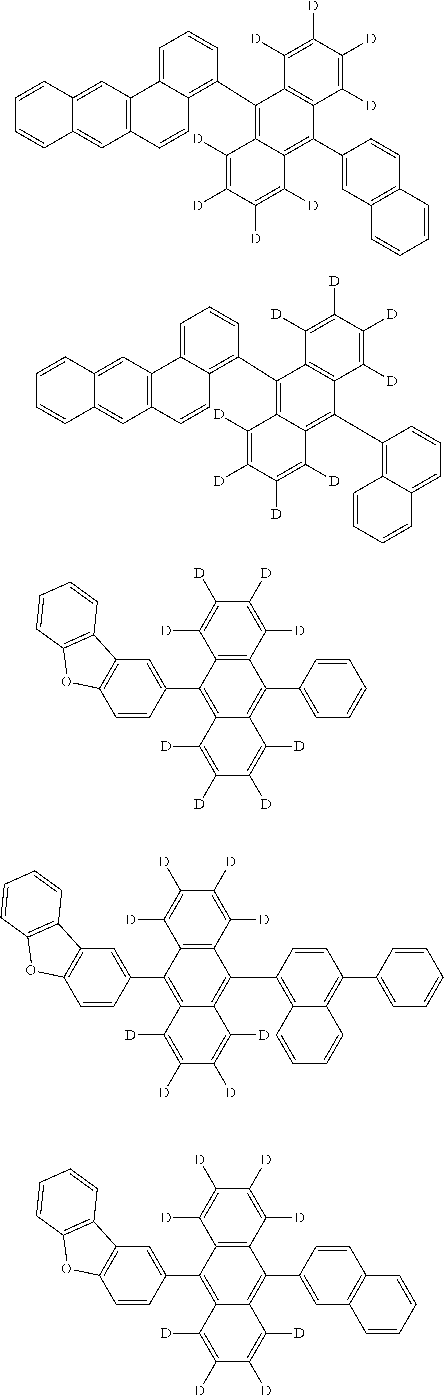

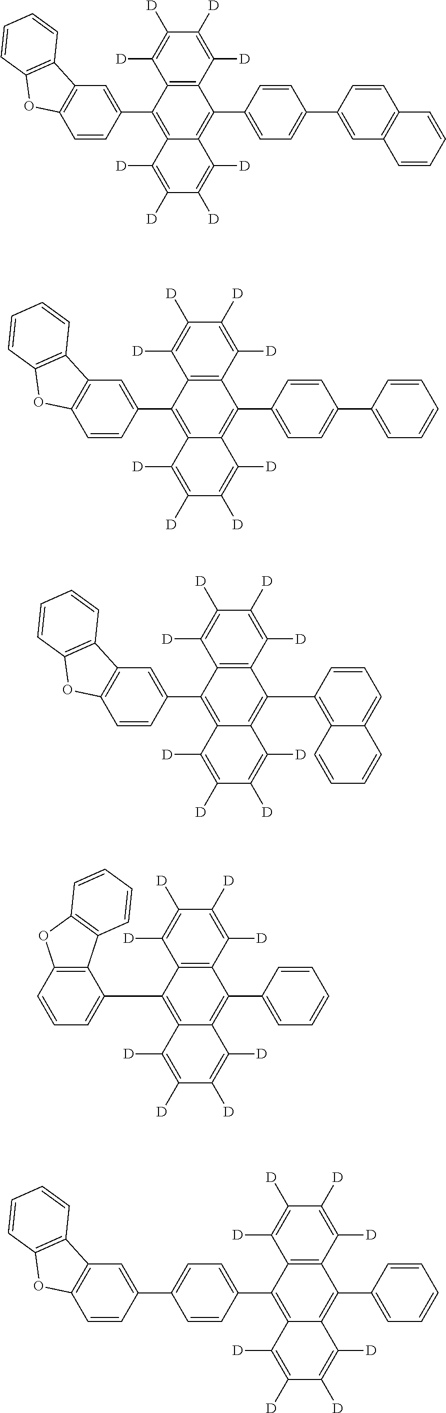

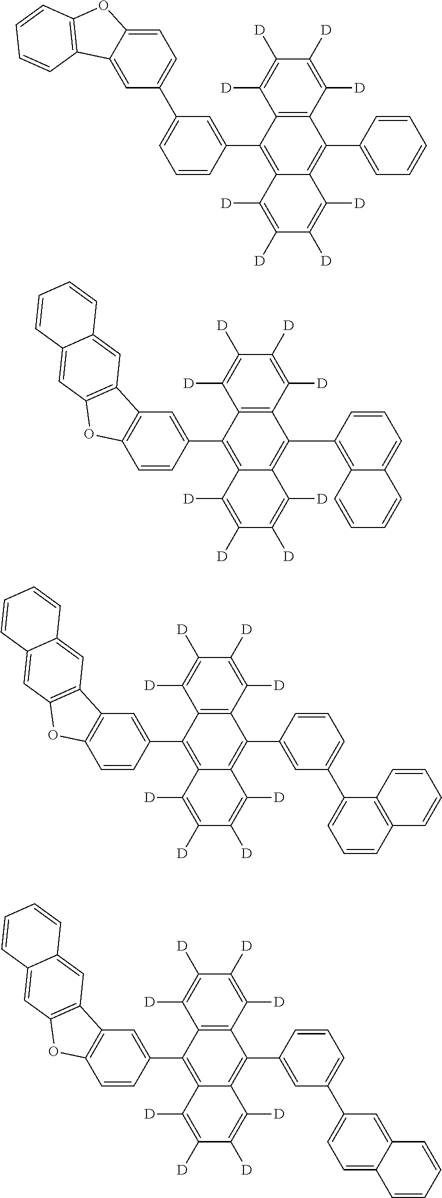 [SMILES: [2H]c1c([2H])c([2H])c2c(-c3cccc(-c4ccc5oc6ccccc6c5c4)c3)c3c([2H])c([2H])c([2H])c([2H])c3c(-c3ccccc3)c2c1[2H] ; [2H]c1c([2H])c([2H])c2c(-c3cccc4ccccc34)c3c([2H])c([2H])c([2H])c([2H])c3c(-c3ccc4oc5cc6ccccc6cc5c4c3)c2c1[2H] ; [2H]c1c([2H])c([2H])c2c(-c3ccc4oc5cc6ccccc6cc5c4c3)c3c([2H])c([2H])c([2H])c([2H])c3c(-c3cccc(-c4cccc5ccccc45)c3)c2c1[2H] ; [2H]c1c([2H])c([2H])c2c(-c3ccc4oc5cc6ccccc6cc5c4c3)c3c([2H])c([2H])c([2H])c([2H])c3c(-c3cccc(-c4ccc5ccccc5c4)c3)c2c1[2H]]

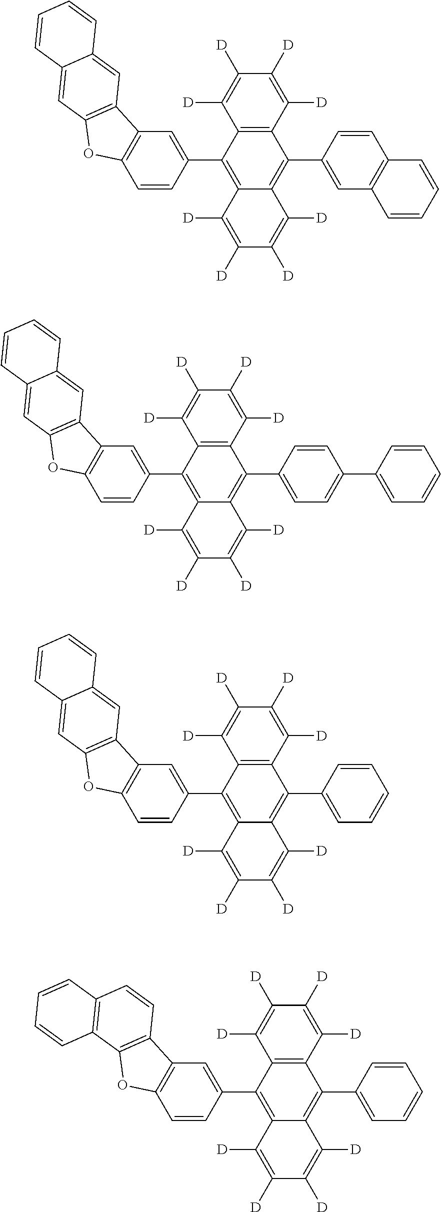 [SMILES: [2H]c1c([2H])c([2H])c2c(-c3ccc4oc5cc6ccccc6cc5c4c3)c3c([2H])c([2H])c([2H])c([2H])c3c(-c3ccc4ccccc4c3)c2c1[2H] ; [2H]c1c([2H])c([2H])c2c(-c3ccc4oc5cc6ccccc6cc5c4c3)c3c([2H])c([2H])c([2H])c([2H])c3c(-c3ccc(-c4ccccc4)cc3)c2c1[2H] ; [2H]c1c([2H])c([2H])c2c(-c3ccc4oc5cc6ccccc6cc5c4c3)c3c([2H])c([2H])c([2H])c([2H])c3c(-c3ccccc3)c2c1[2H] ; [2H]c1c([2H])c([2H])c2c(-c3ccc4oc5c6ccccc6ccc5c4c3)c3c([2H])c([2H])c([2H])c([2H])c3c(-c3ccccc3)c2c1[2H]]

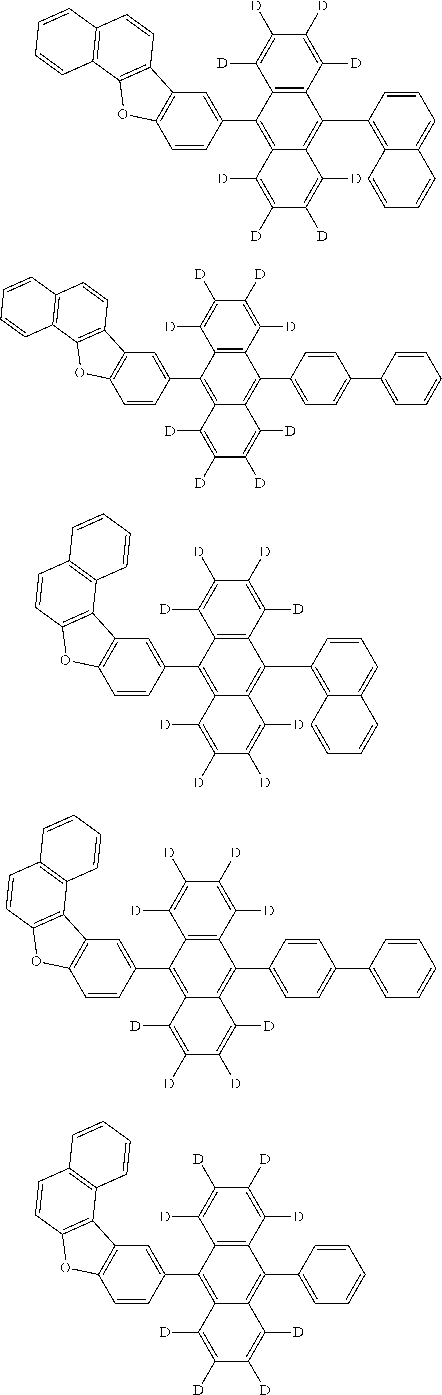

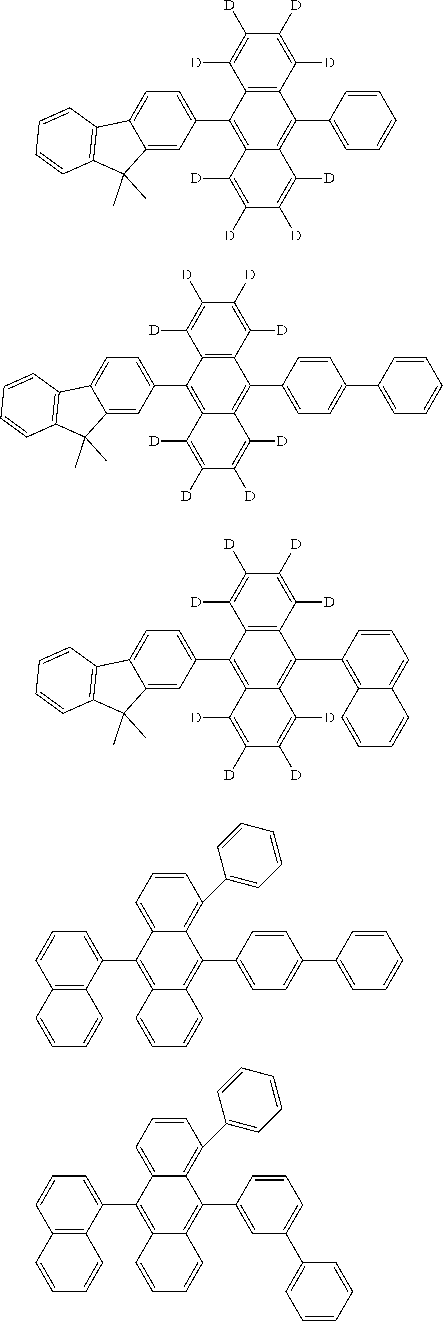

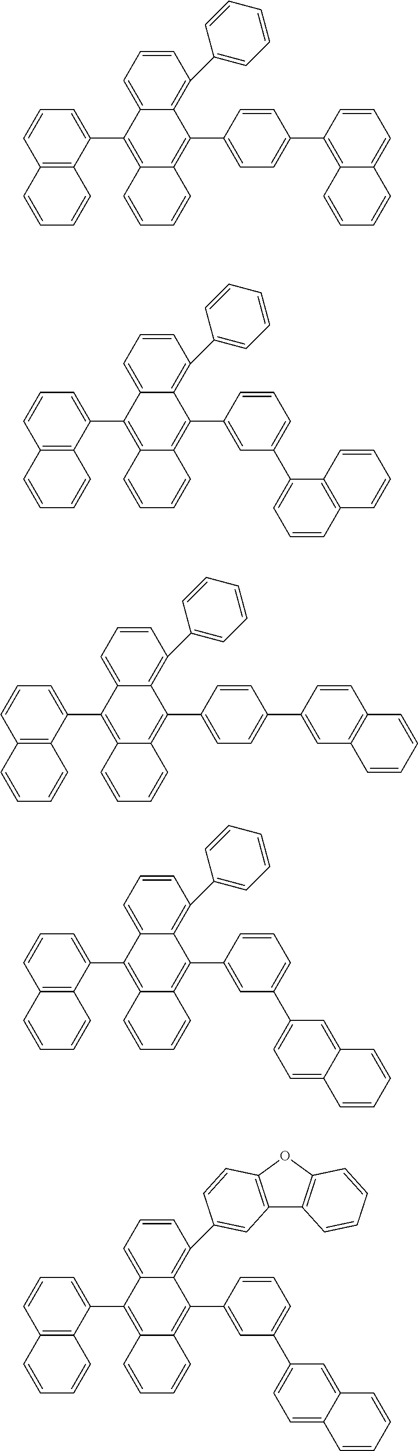

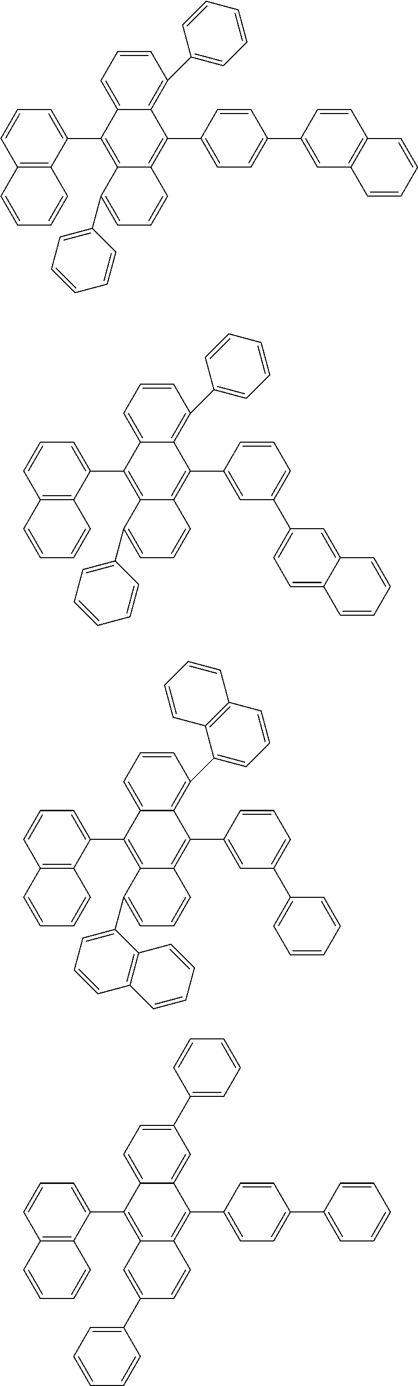

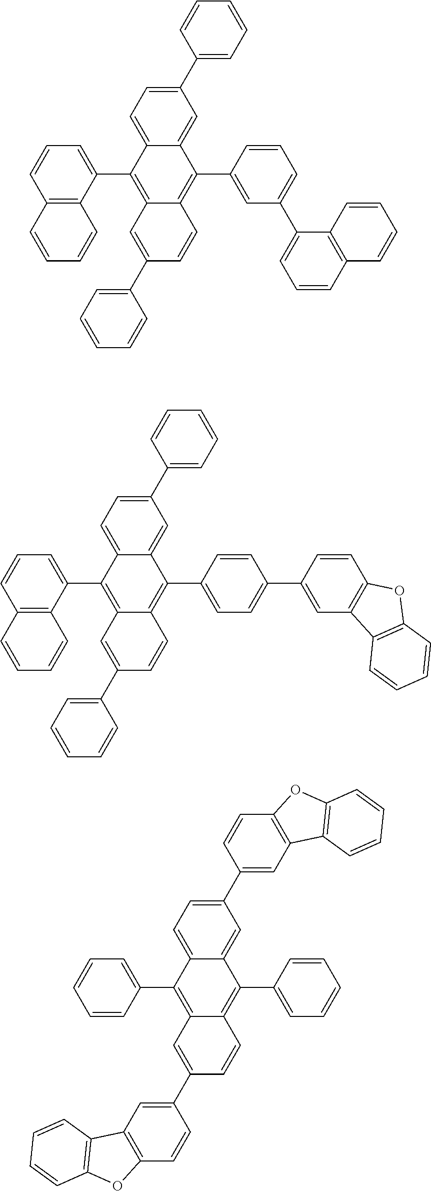

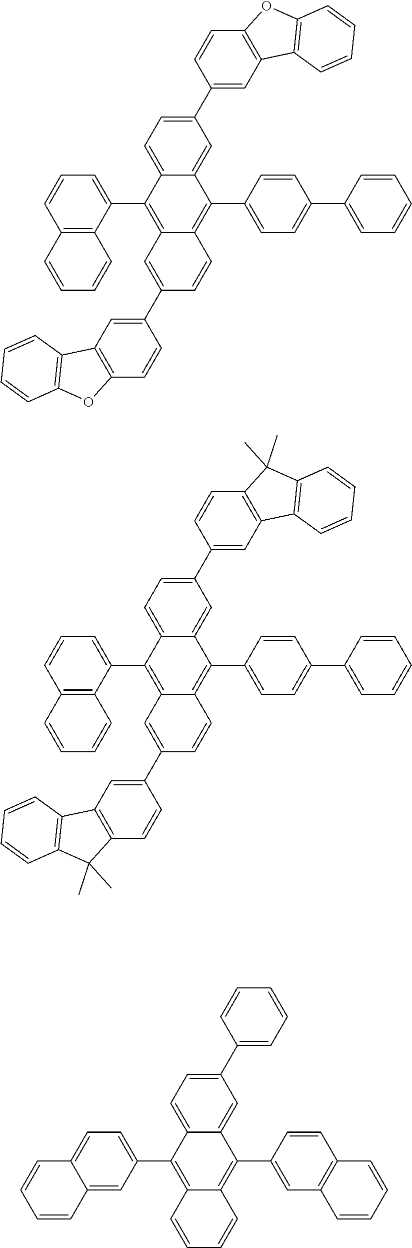

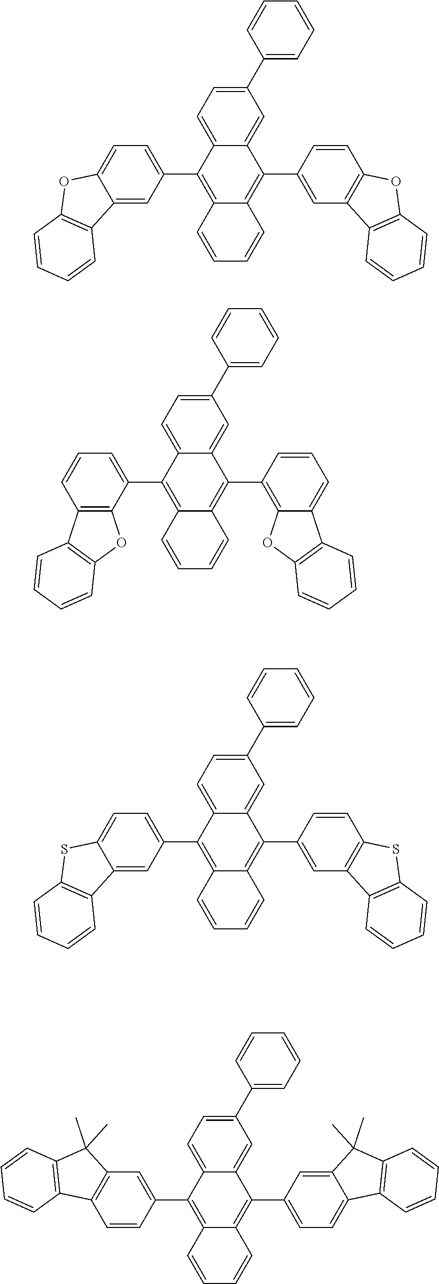

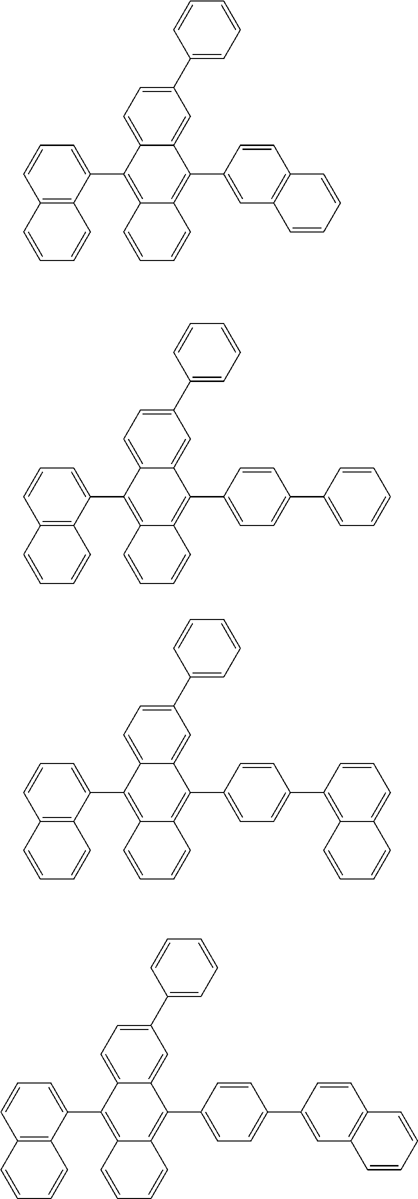

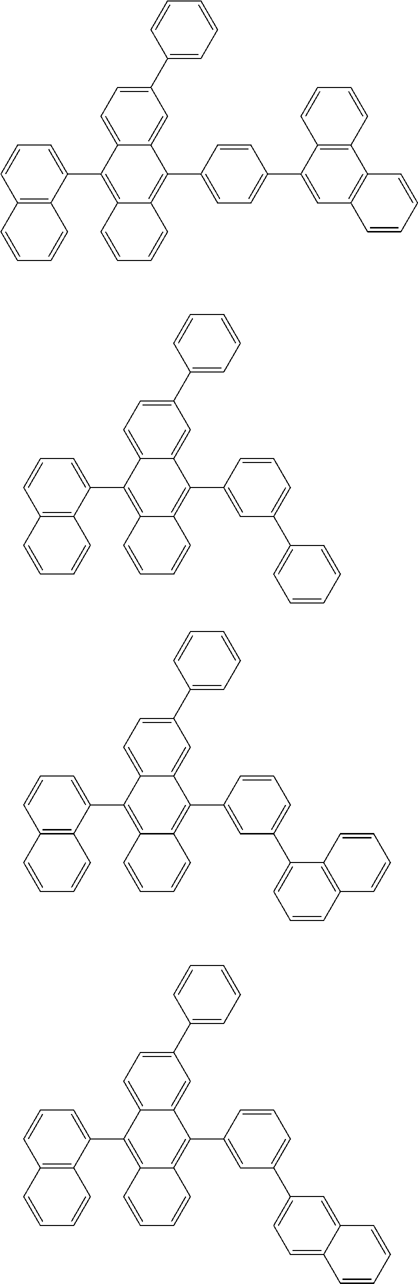

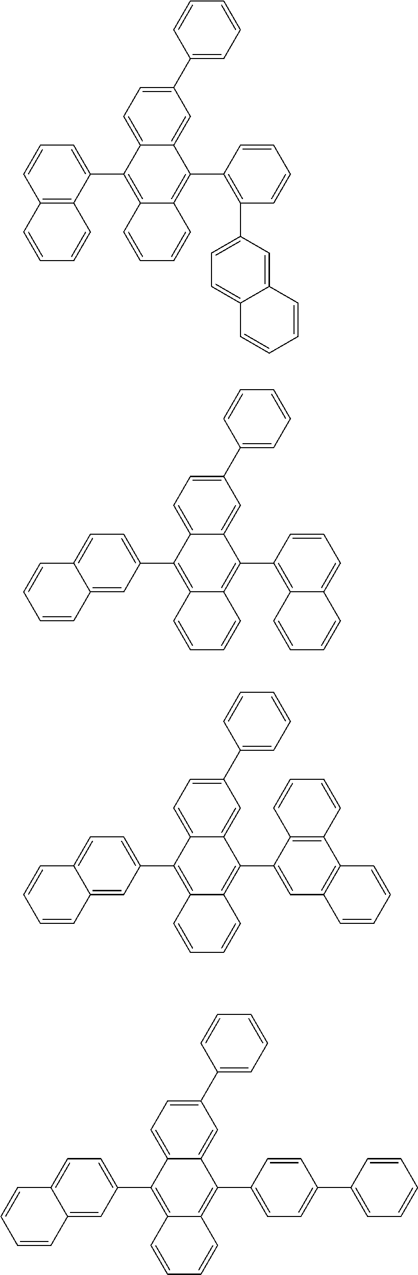

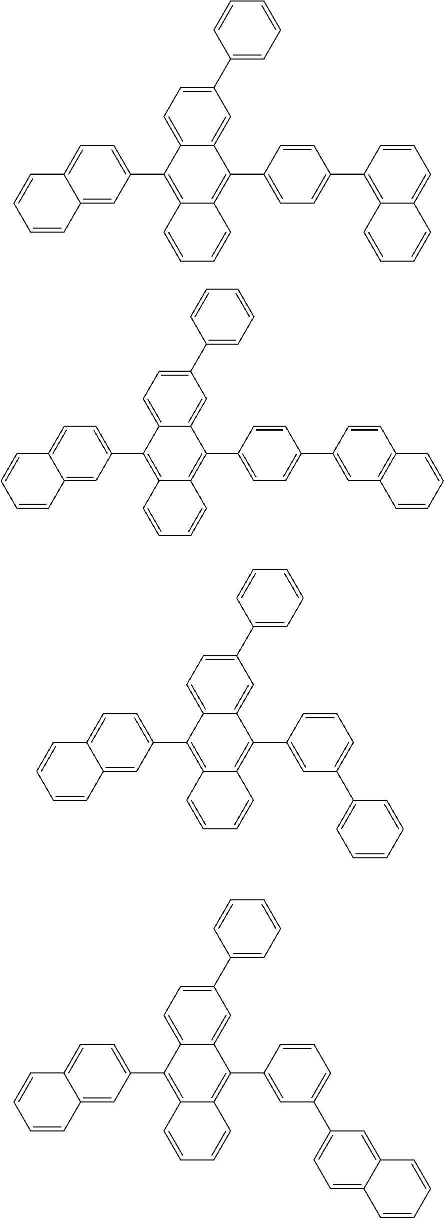

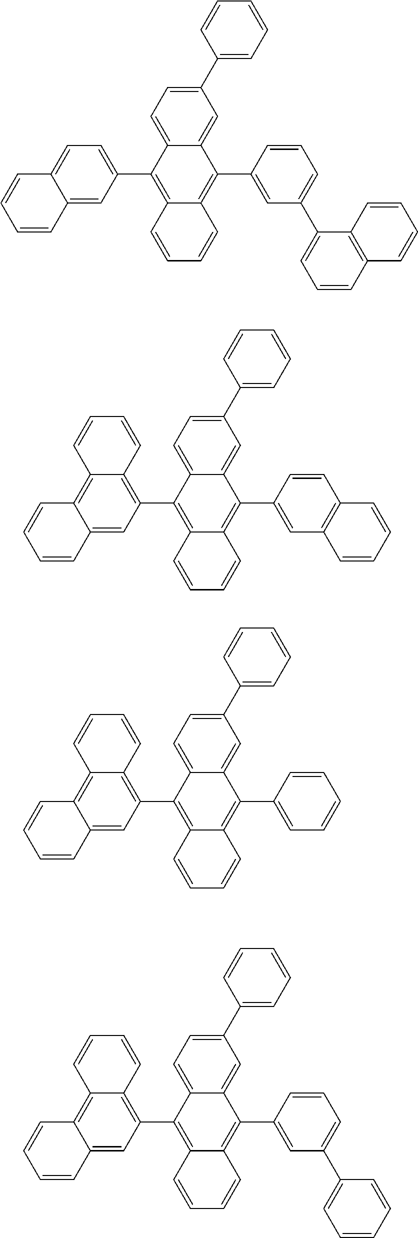

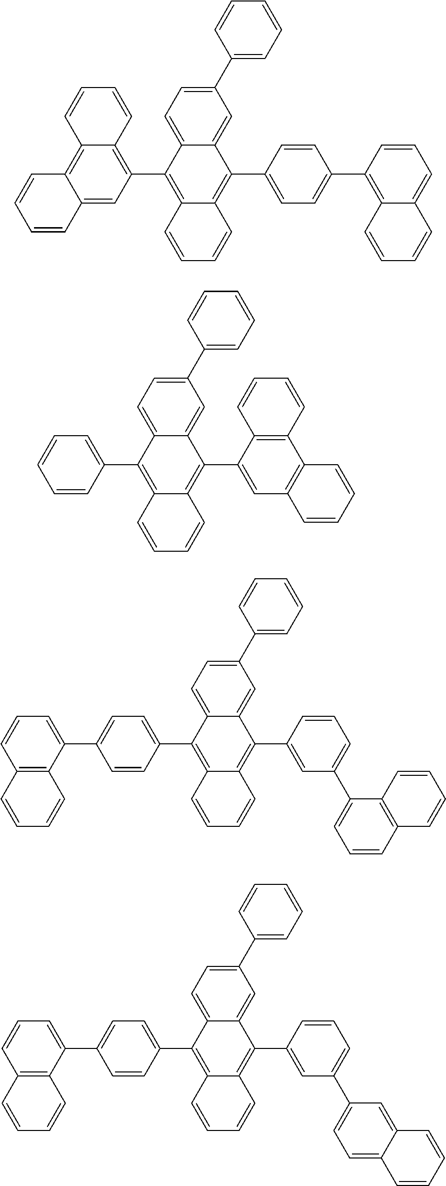

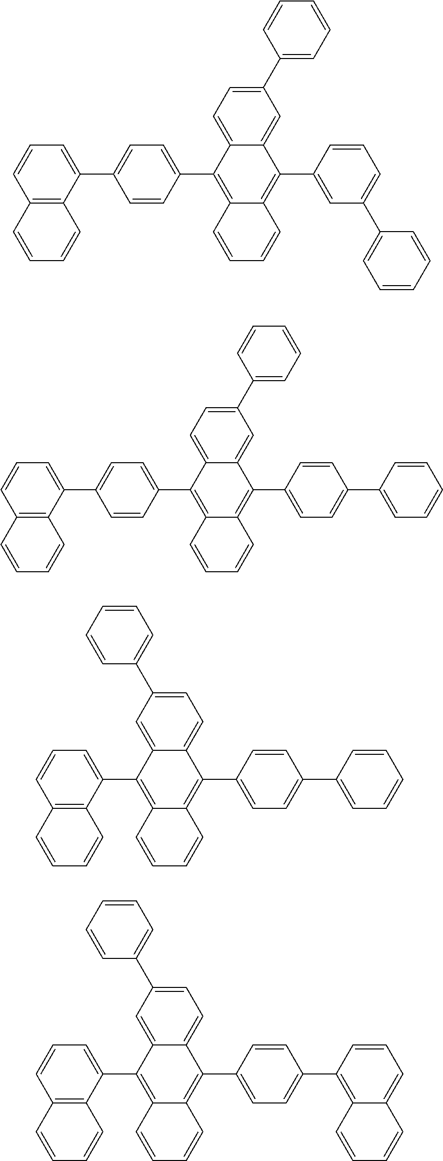

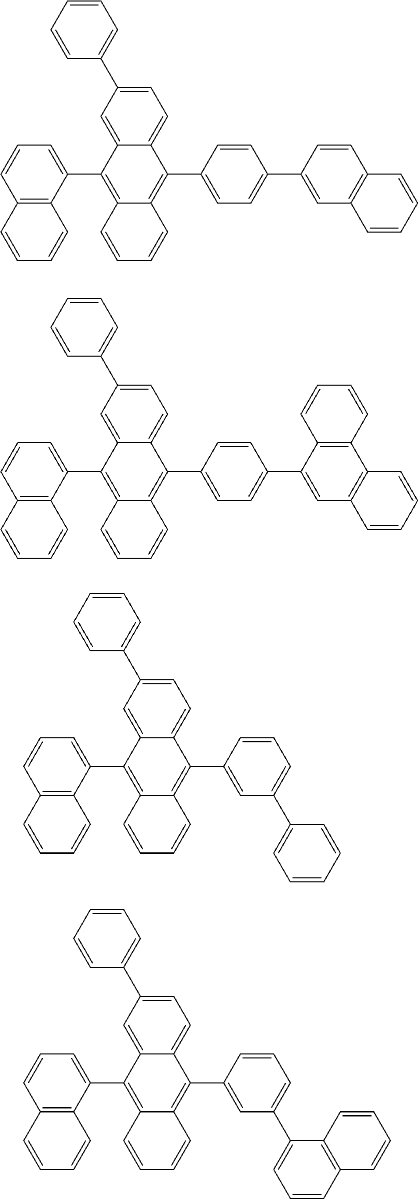

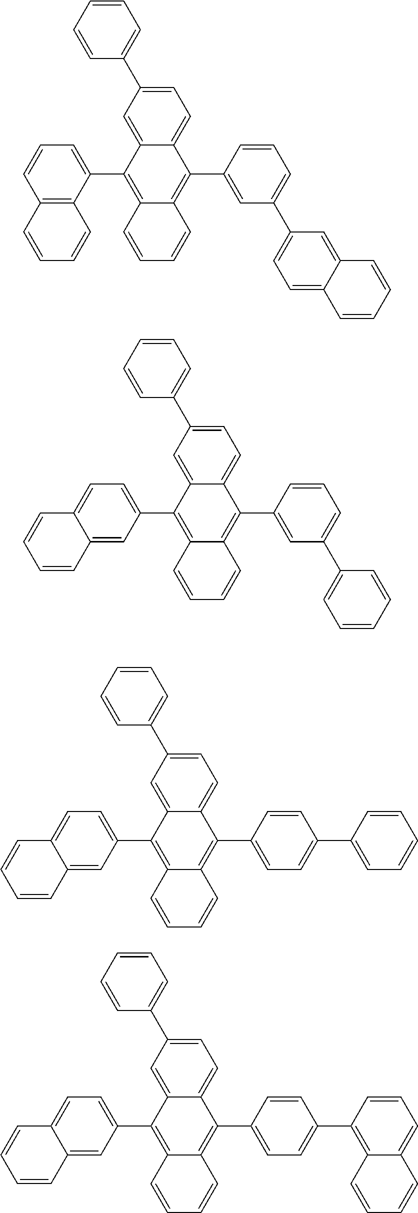

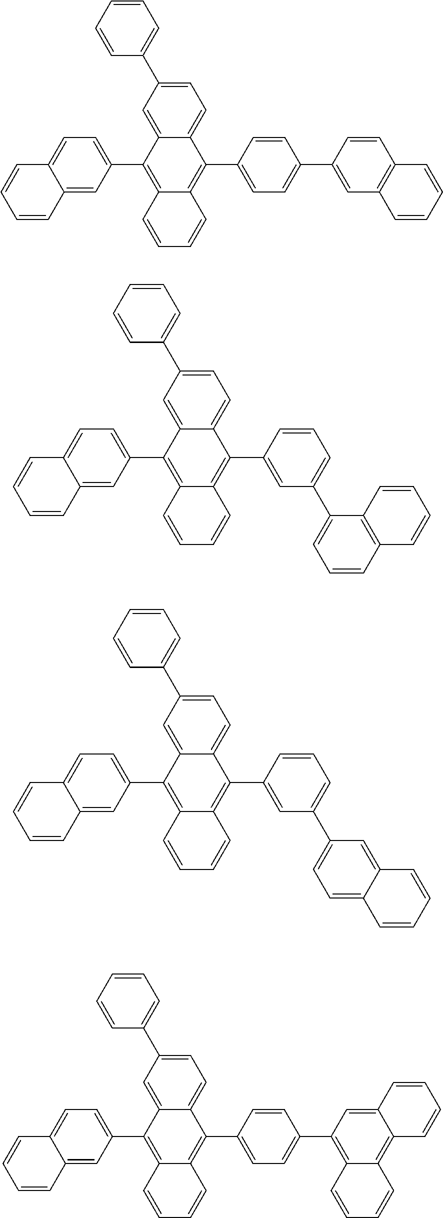

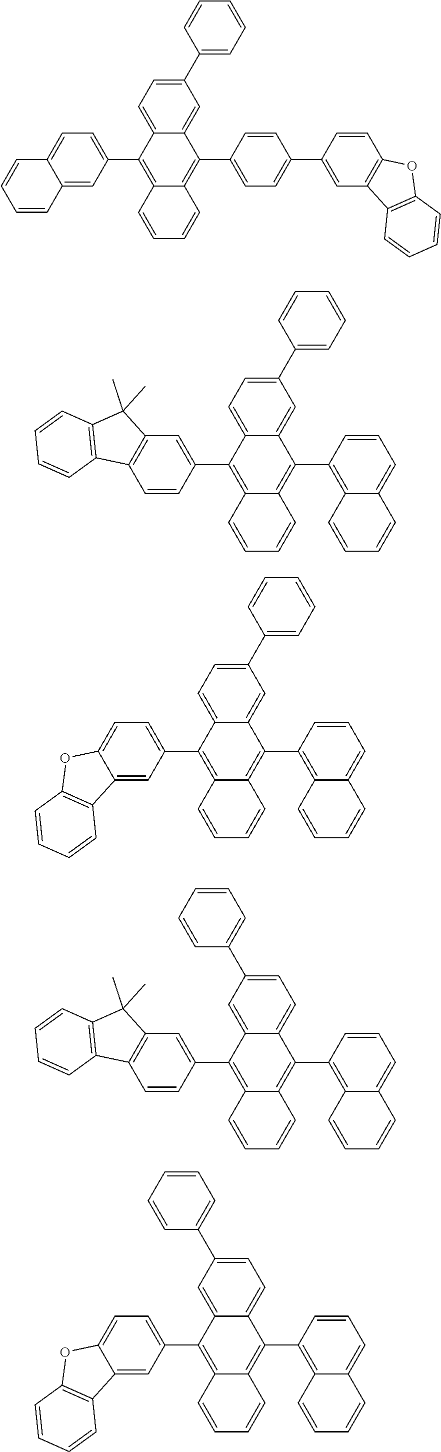

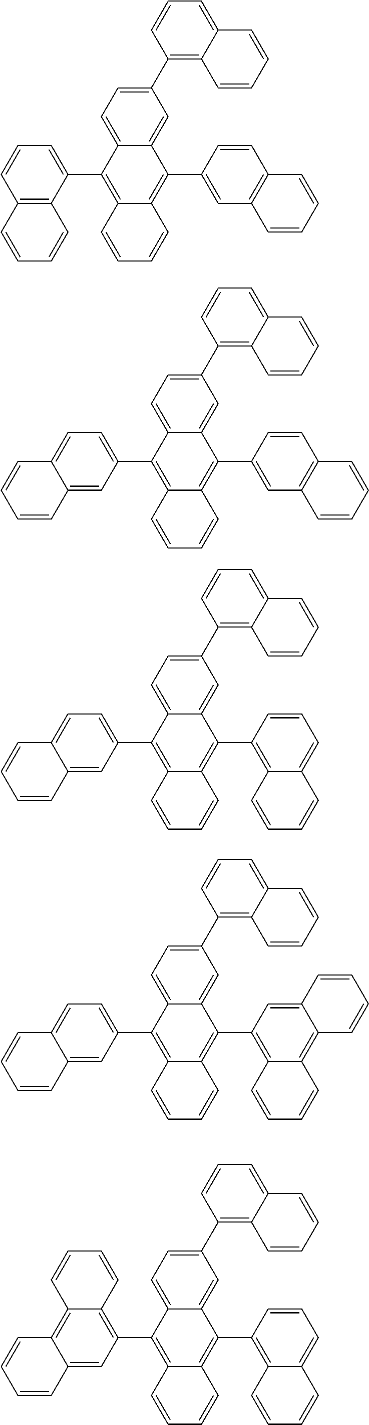

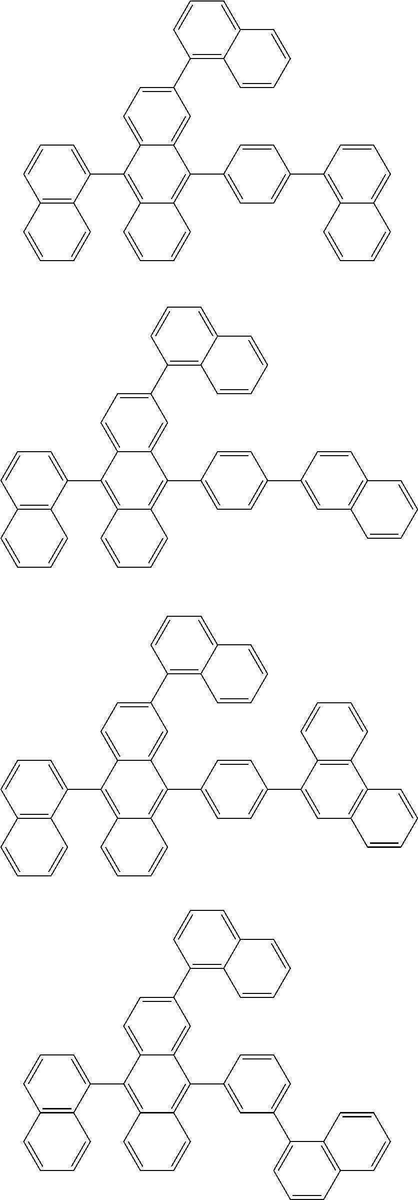

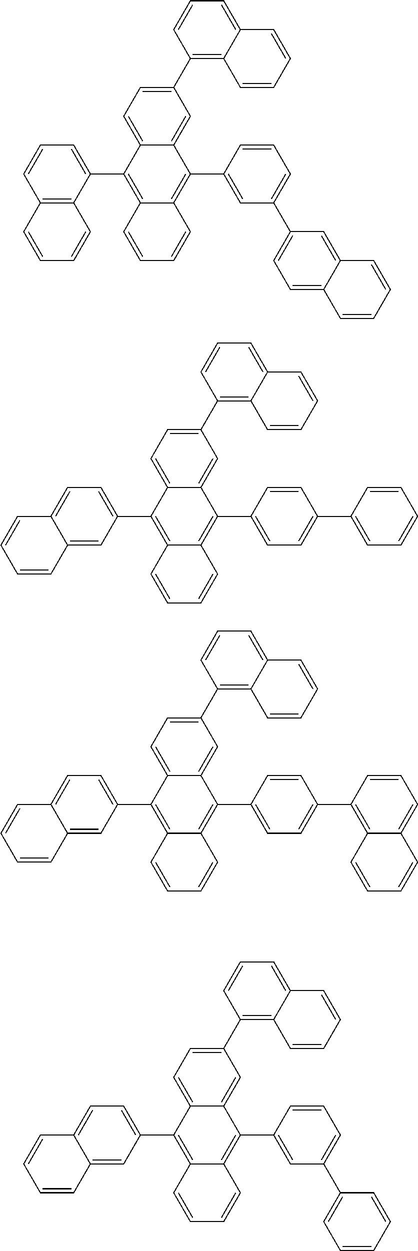

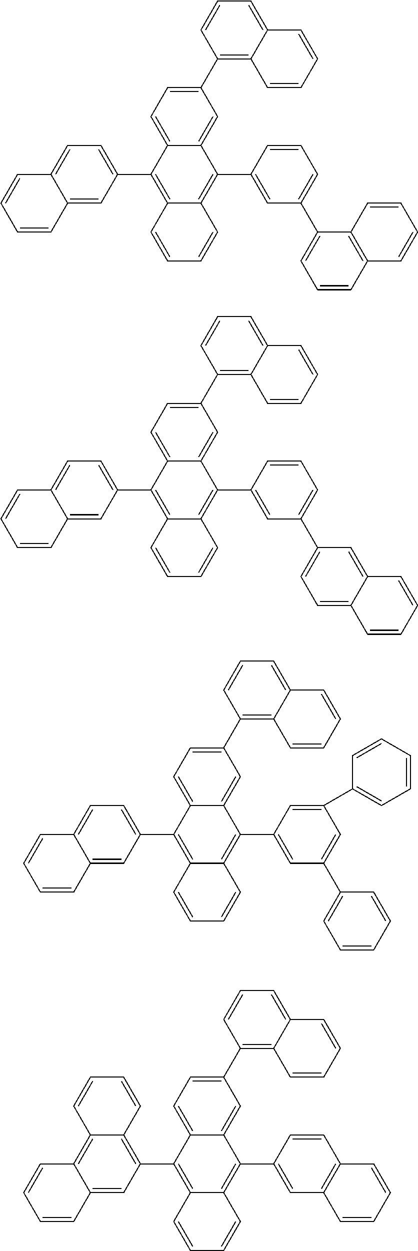

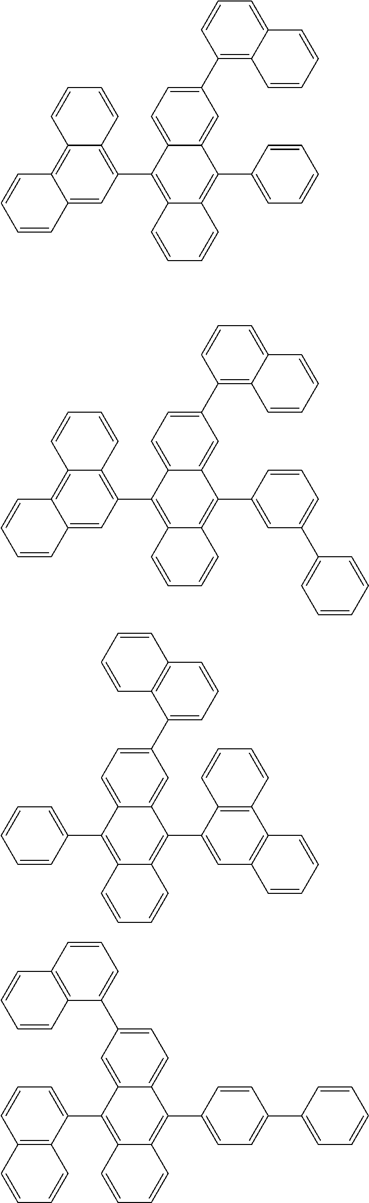

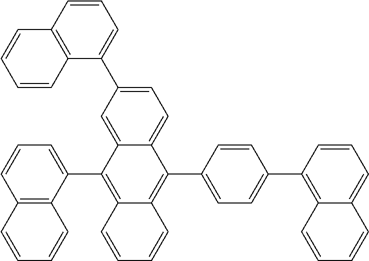 [SMILES: c1ccc2c(-c3ccc(-c4c5ccccc5c(-c5cccc6ccccc56)c5cc(-c6cccc7ccccc67)ccc45)cc3)cccc2c1]

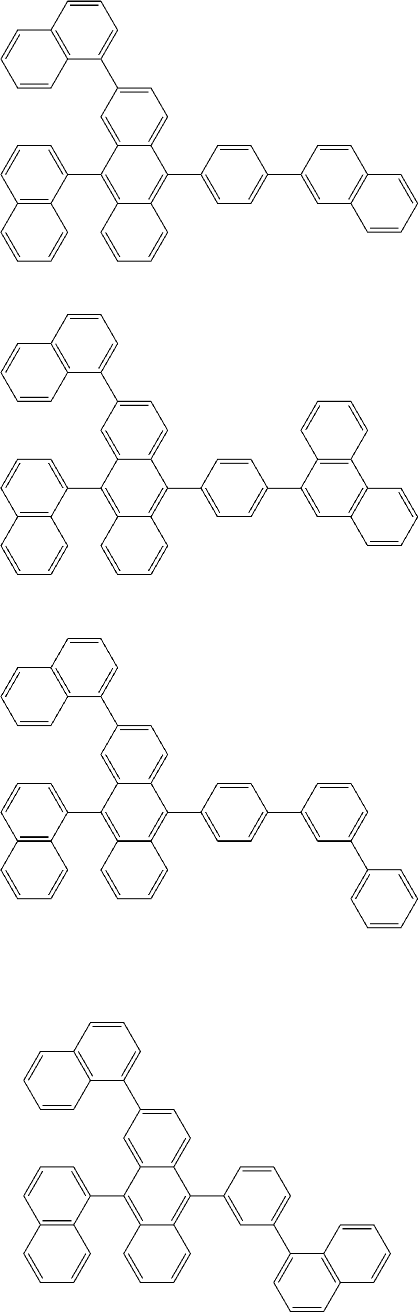

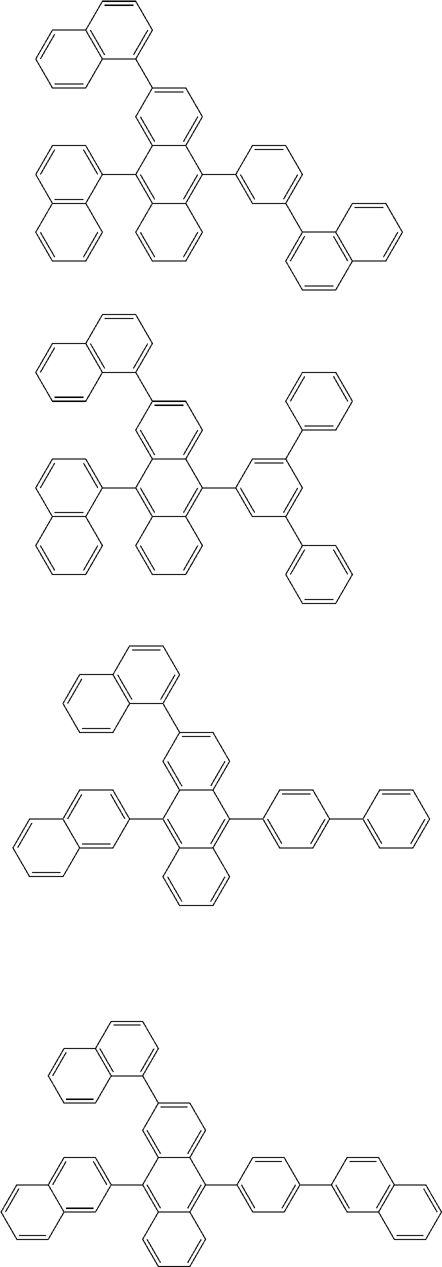

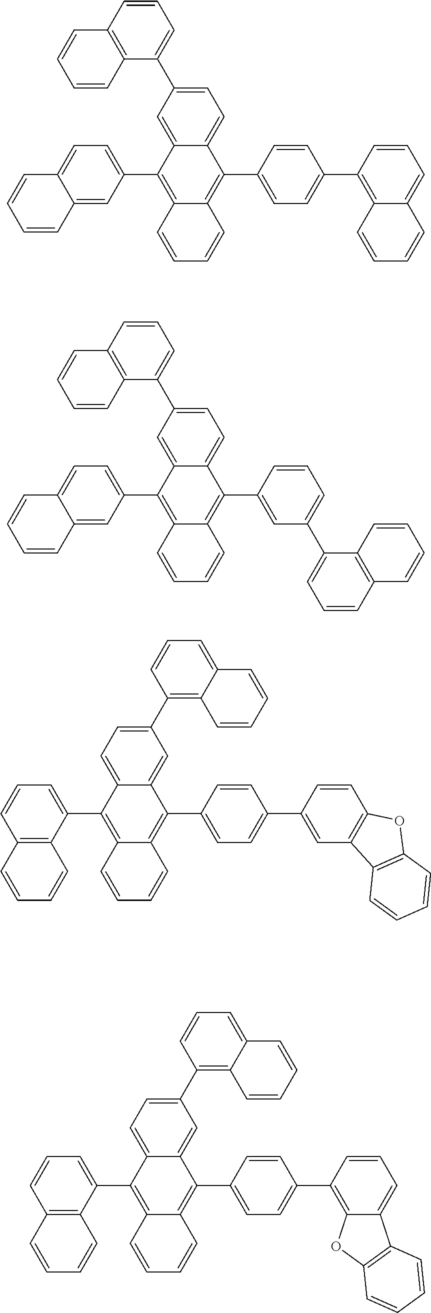

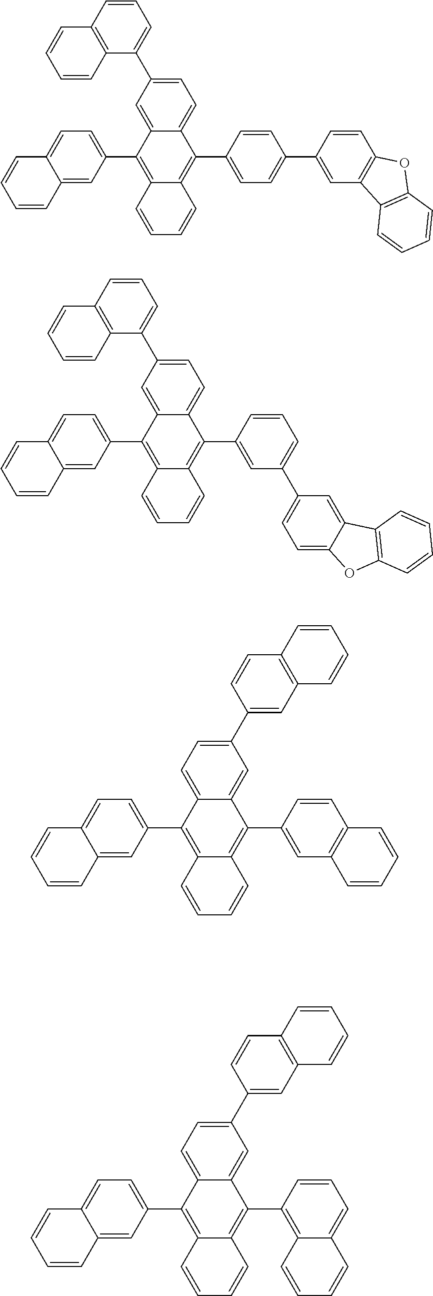

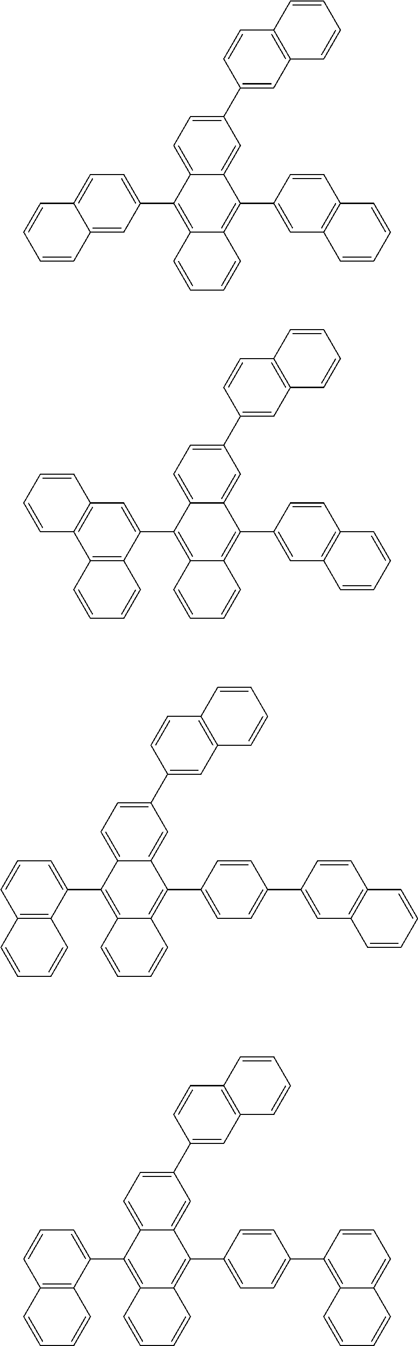

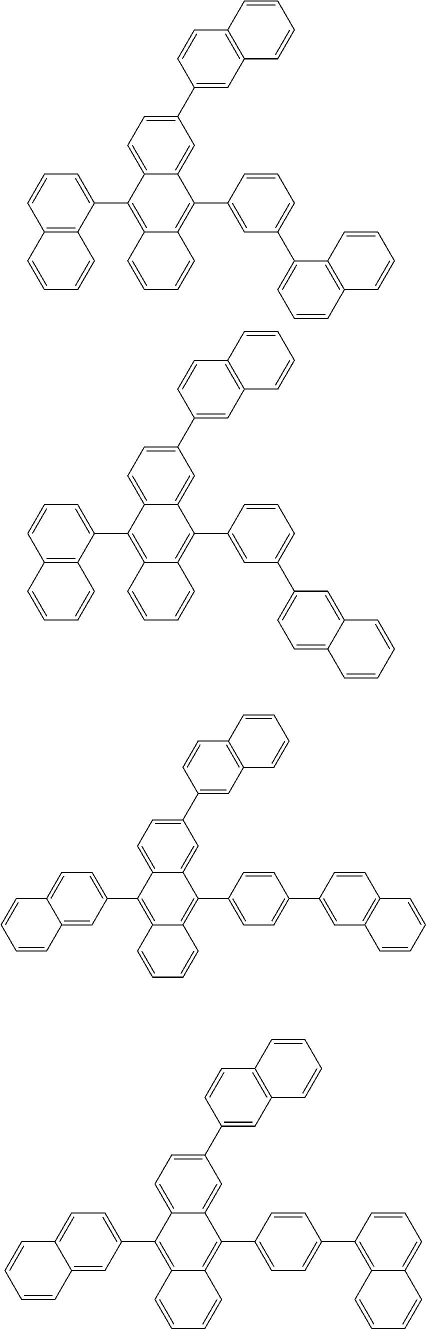

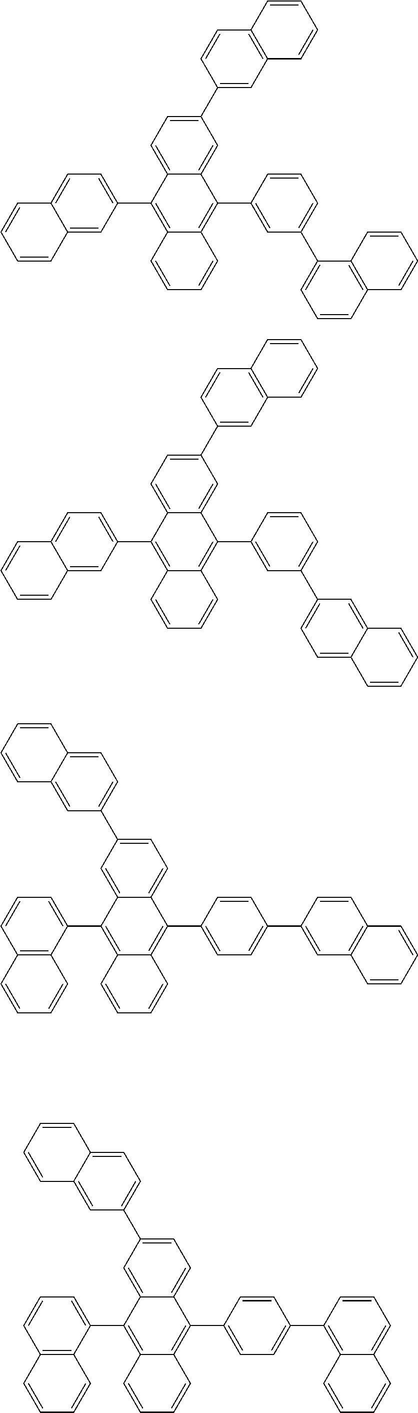

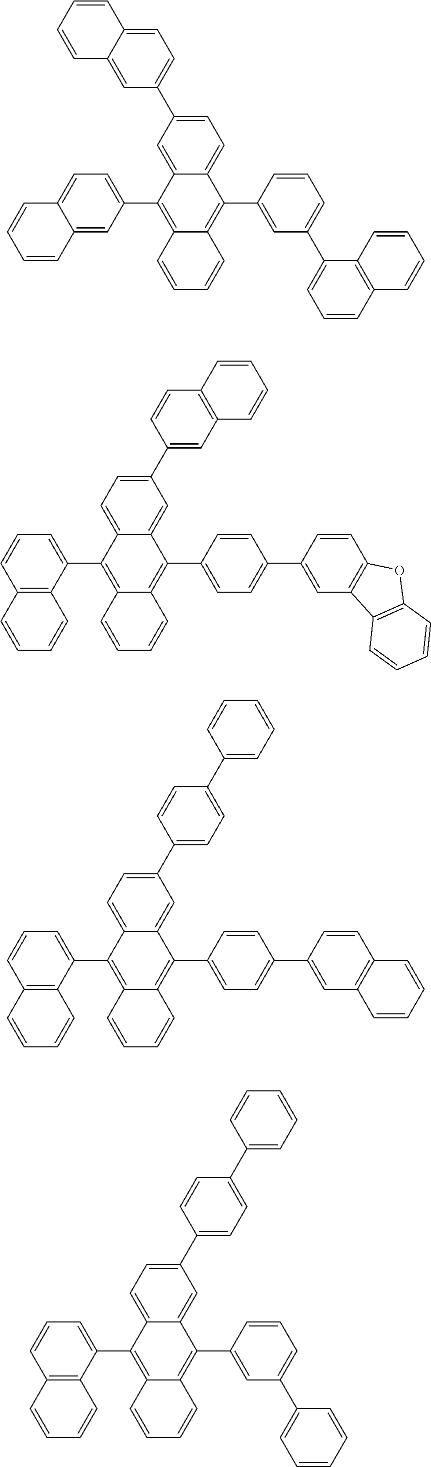

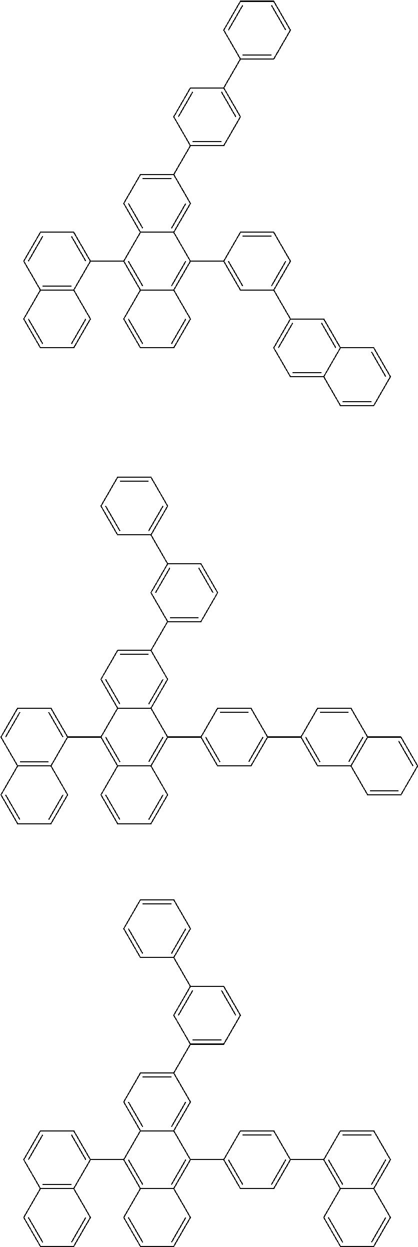

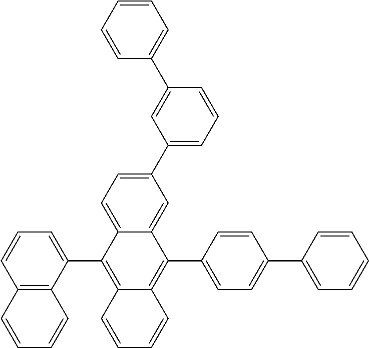 [SMILES: c1ccc(-c2ccc(-c3c4ccccc4c(-c4cccc5ccccc45)c4ccc(-c5cccc(-c6ccccc6)c5)cc34)cc2)cc1]

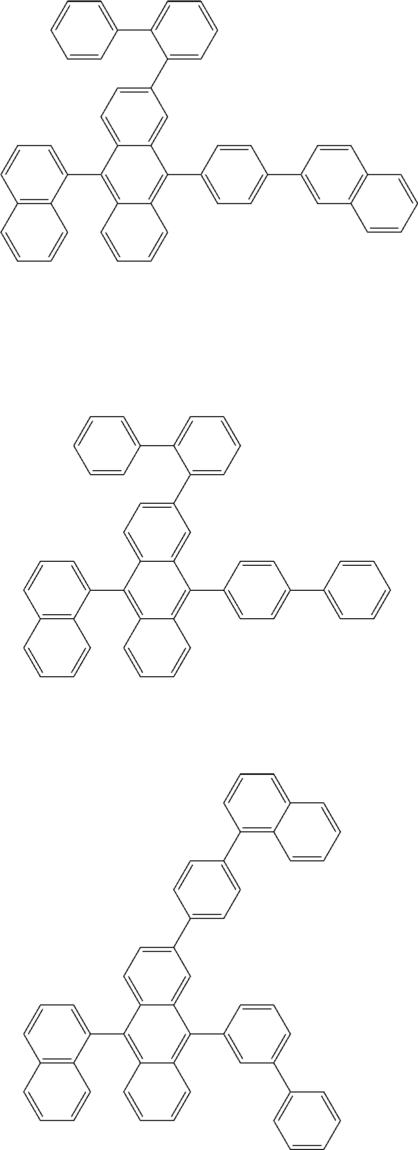

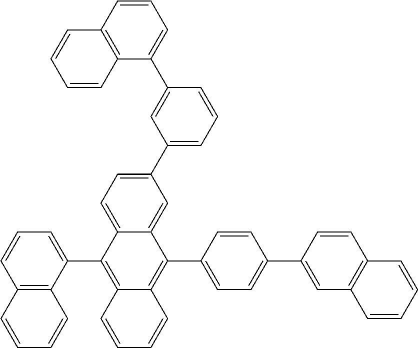 [SMILES: c1cc(-c2ccc3c(-c4cccc5ccccc45)c4ccccc4c(-c4ccc(-c5ccc6ccccc6c5)cc4)c3c2)cc(-c2cccc3ccccc23)c1]

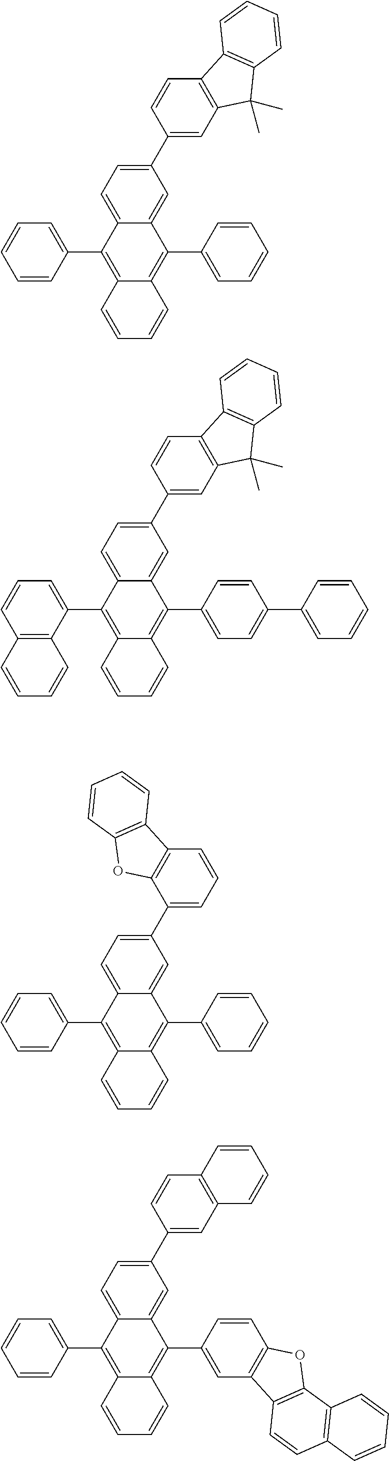

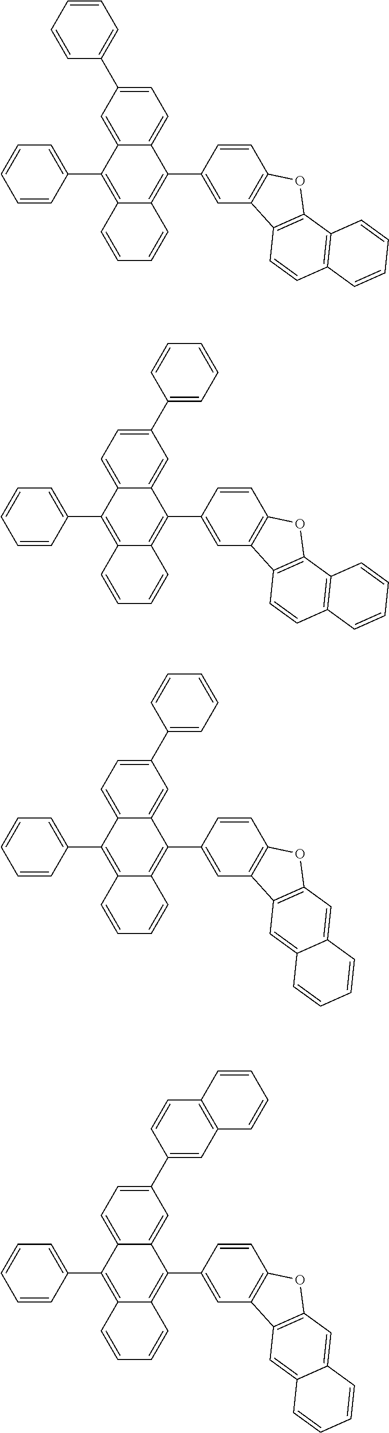 [SMILES: c1ccc(-c2ccc3c(-c4ccc5oc6c7ccccc7ccc6c5c4)c4ccccc4c(-c4ccccc4)c3c2)cc1 ; c1ccc(-c2ccc3c(-c4ccccc4)c4ccccc4c(-c4ccc5oc6c7ccccc7ccc6c5c4)c3c2)cc1 ; c1ccc(-c2ccc3c(-c4ccccc4)c4ccccc4c(-c4ccc5oc6cc7ccccc7cc6c5c4)c3c2)cc1 ; c1ccc(-c2c3ccccc3c(-c3ccc4oc5cc6ccccc6cc5c4c3)c3cc(-c4ccc5ccccc5c4)ccc23)cc1]

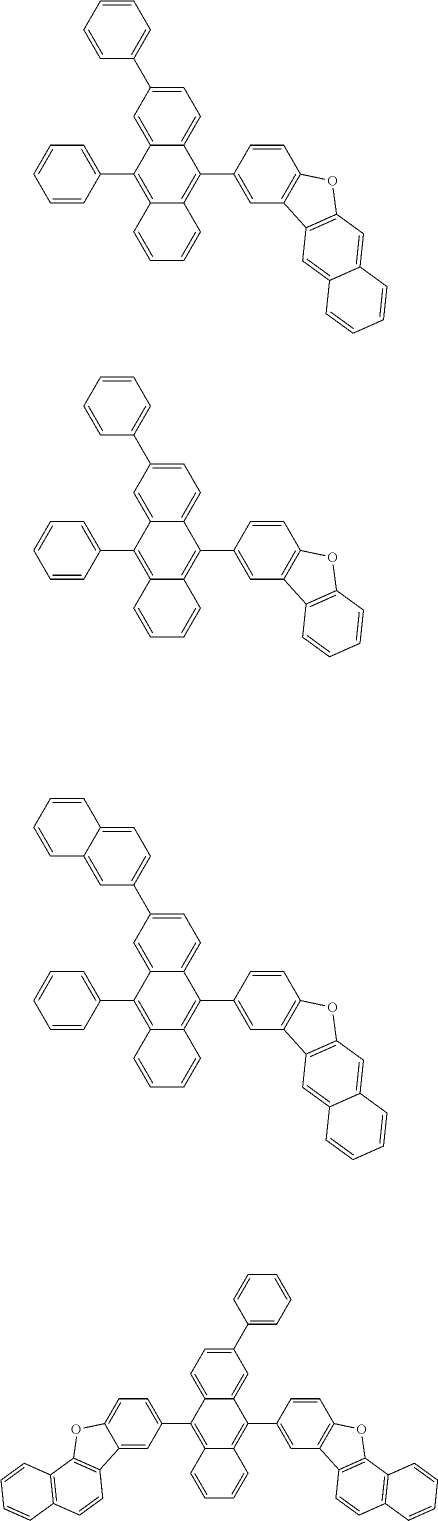 [SMILES: c1ccc(-c2ccc3c(-c4ccc5oc6cc7ccccc7cc6c5c4)c4ccccc4c(-c4ccccc4)c3c2)cc1 ; c1ccc(-c2ccc3c(-c4ccc5oc6ccccc6c5c4)c4ccccc4c(-c4ccccc4)c3c2)cc1 ; c1ccc(-c2c3ccccc3c(-c3ccc4oc5cc6ccccc6cc5c4c3)c3ccc(-c4ccc5ccccc5c4)cc23)cc1 ; c1ccc(-c2ccc3c(-c4ccc5oc6c7ccccc7ccc6c5c4)c4ccccc4c(-c4ccc5oc6c7ccccc7ccc6c5c4)c3c2)cc1]

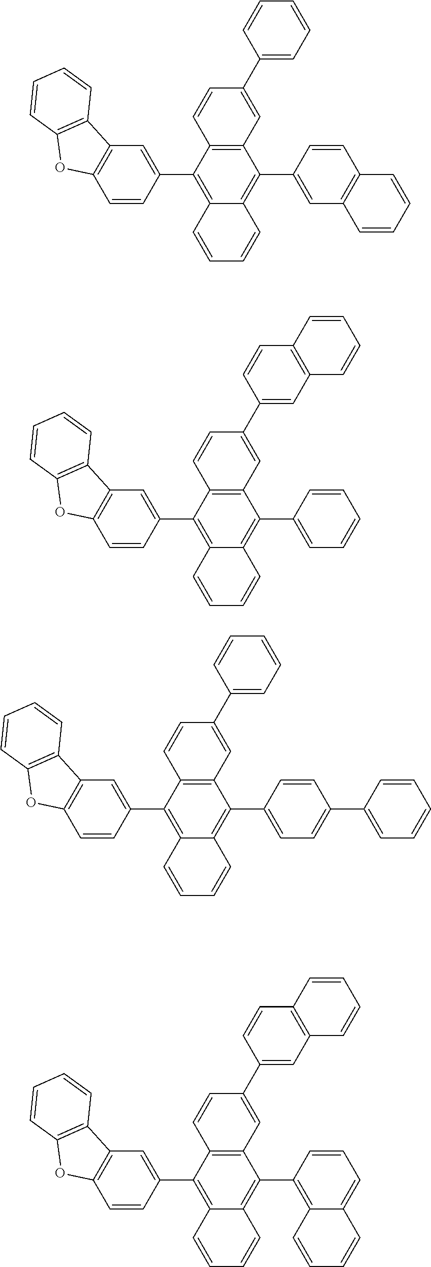

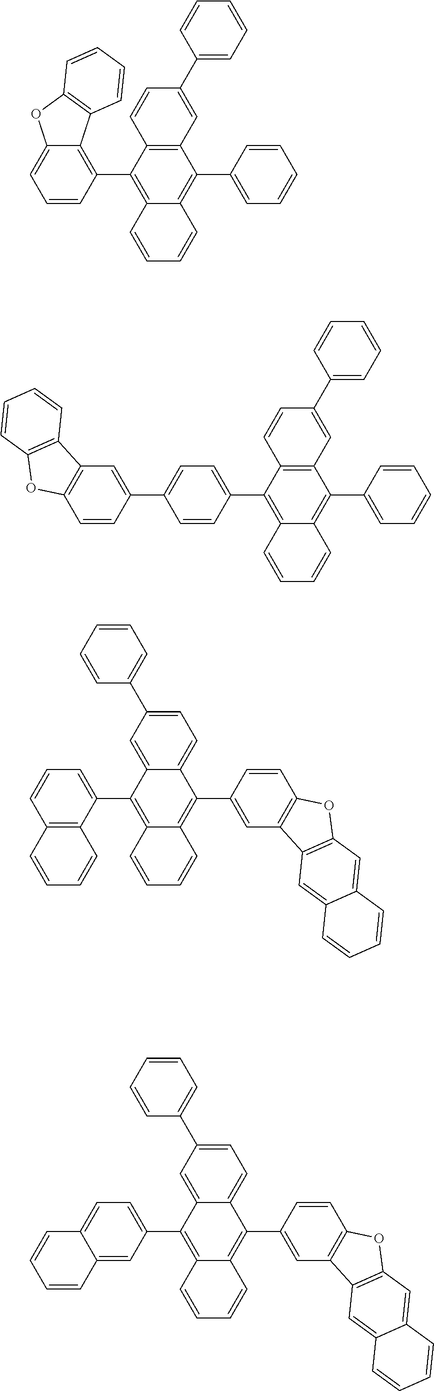 [SMILES: c1ccc(-c2ccc3c(-c4cccc5oc6ccccc6c45)c4ccccc4c(-c4ccccc4)c3c2)cc1 ; c1ccc(-c2ccc3c(-c4ccc(-c5ccc6oc7ccccc7c6c5)cc4)c4ccccc4c(-c4ccccc4)c3c2)cc1 ; c1ccc(-c2ccc3c(-c4ccc5oc6cc7ccccc7cc6c5c4)c4ccccc4c(-c4cccc5ccccc45)c3c2)cc1 ; c1ccc(-c2ccc3c(-c4ccc5oc6cc7ccccc7cc6c5c4)c4ccccc4c(-c4ccc5ccccc5c4)c3c2)cc1]

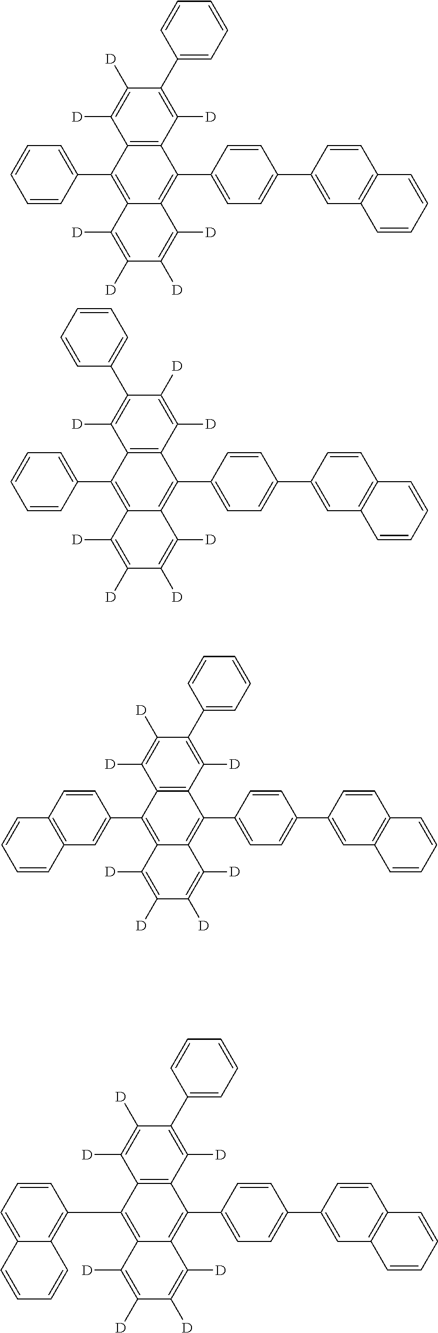

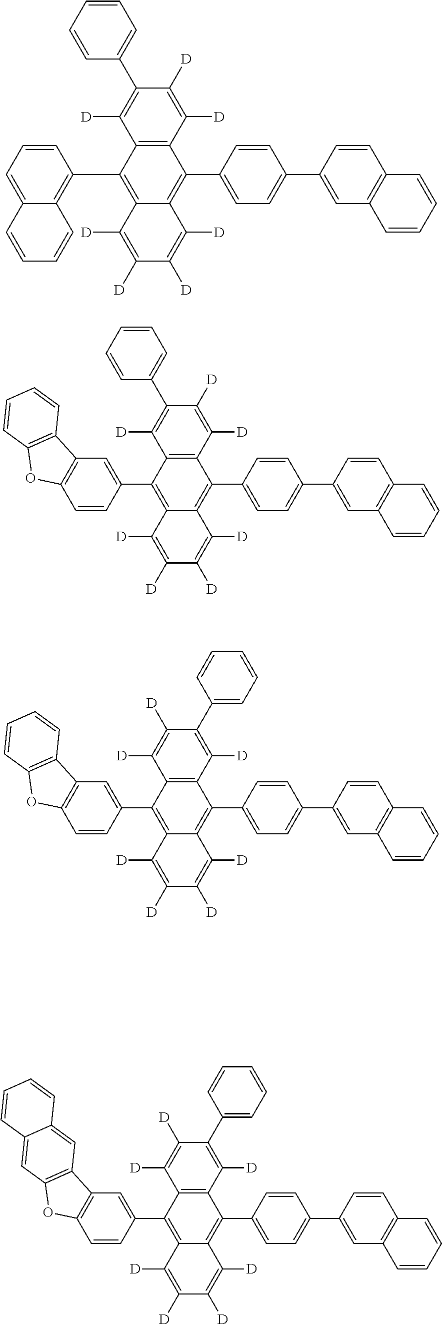

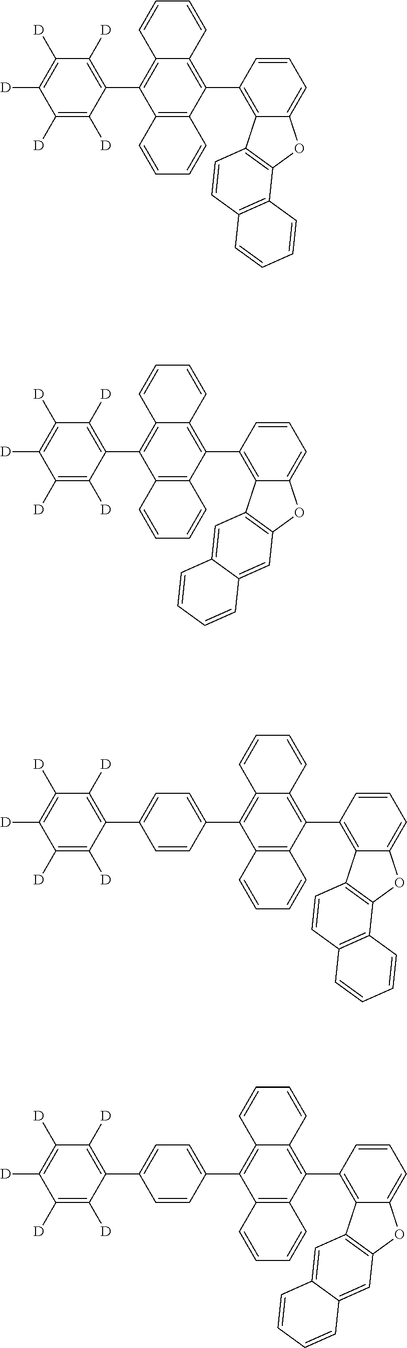 [SMILES: [2H]c1c([2H])c([2H])c(-c2c3ccccc3c(-c3cccc4oc5c6ccccc6ccc5c34)c3ccccc23)c([2H])c1[2H] ; [2H]c1c([2H])c([2H])c(-c2c3ccccc3c(-c3cccc4oc5cc6ccccc6cc5c34)c3ccccc23)c([2H])c1[2H] ; [2H]c1c([2H])c([2H])c(-c2ccc(-c3c4ccccc4c(-c4cccc5oc6c7ccccc7ccc6c45)c4ccccc34)cc2)c([2H])c1[2H] ; [2H]c1c([2H])c([2H])c(-c2ccc(-c3c4ccccc4c(-c4cccc5oc6cc7ccccc7cc6c45)c4ccccc34)cc2)c([2H])c1[2H]]

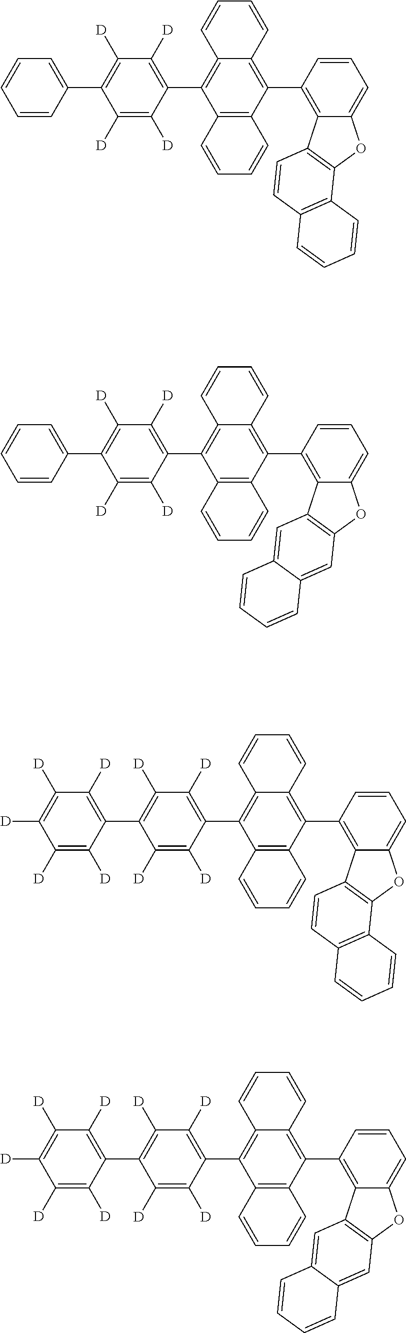 [SMILES: [2H]c1c([2H])c(-c2c3ccccc3c(-c3cccc4oc5c6ccccc6ccc5c34)c3ccccc23)c([2H])c([2H])c1-c1ccccc1 ; [2H]c1c([2H])c(-c2c3ccccc3c(-c3cccc4oc5cc6ccccc6cc5c34)c3ccccc23)c([2H])c([2H])c1-c1ccccc1 ; [2H]c1c([2H])c([2H])c(-c2c([2H])c([2H])c(-c3c4ccccc4c(-c4cccc5oc6c7ccccc7ccc6c45)c4ccccc34)c([2H])c2[2H])c([2H])c1[2H] ; [2H]c1c([2H])c([2H])c(-c2c([2H])c([2H])c(-c3c4ccccc4c(-c4cccc5oc6cc7ccccc7cc6c45)c4ccccc34)c([2H])c2[2H])c([2H])c1[2H]]

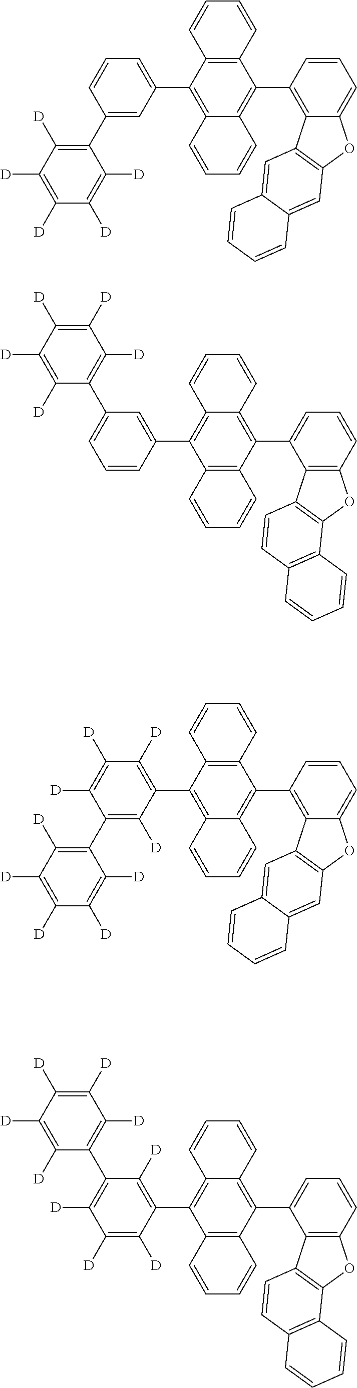 [SMILES: [2H]c1c([2H])c([2H])c(-c2cccc(-c3c4ccccc4c(-c4cccc5oc6cc7ccccc7cc6c45)c4ccccc34)c2)c([2H])c1[2H] ; [2H]c1c([2H])c([2H])c(-c2cccc(-c3c4ccccc4c(-c4cccc5oc6c7ccccc7ccc6c45)c4ccccc34)c2)c([2H])c1[2H] ; [2H]c1c([2H])c([2H])c(-c2c([2H])c([2H])c([2H])c(-c3c4ccccc4c(-c4cccc5oc6cc7ccccc7cc6c45)c4ccccc34)c2[2H])c([2H])c1[2H] ; [2H]c1c([2H])c([2H])c(-c2c([2H])c([2H])c([2H])c(-c3c4ccccc4c(-c4cccc5oc6c7ccccc7ccc6c45)c4ccccc34)c2[2H])c([2H])c1[2H]]

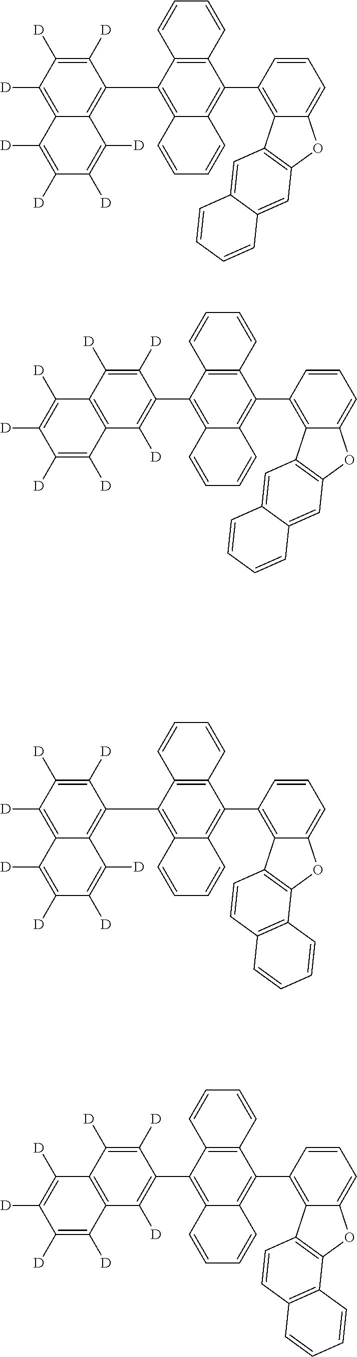 [SMILES: [2H]c1c([2H])c([2H])c2c(-c3c4ccccc4c(-c4cccc5oc6cc7ccccc7cc6c45)c4ccccc34)c([2H])c([2H])c([2H])c2c1[2H] ; [2H]c1c([2H])c([2H])c2c([2H])c(-c3c4ccccc4c(-c4cccc5oc6cc7ccccc7cc6c45)c4ccccc34)c([2H])c([2H])c2c1[2H] ; [2H]c1c([2H])c([2H])c2c(-c3c4ccccc4c(-c4cccc5oc6c7ccccc7ccc6c45)c4ccccc34)c([2H])c([2H])c([2H])c2c1[2H] ; [2H]c1c([2H])c([2H])c2c([2H])c(-c3c4ccccc4c(-c4cccc5oc6c7ccccc7ccc6c45)c4ccccc34)c([2H])c([2H])c2c1[2H]]

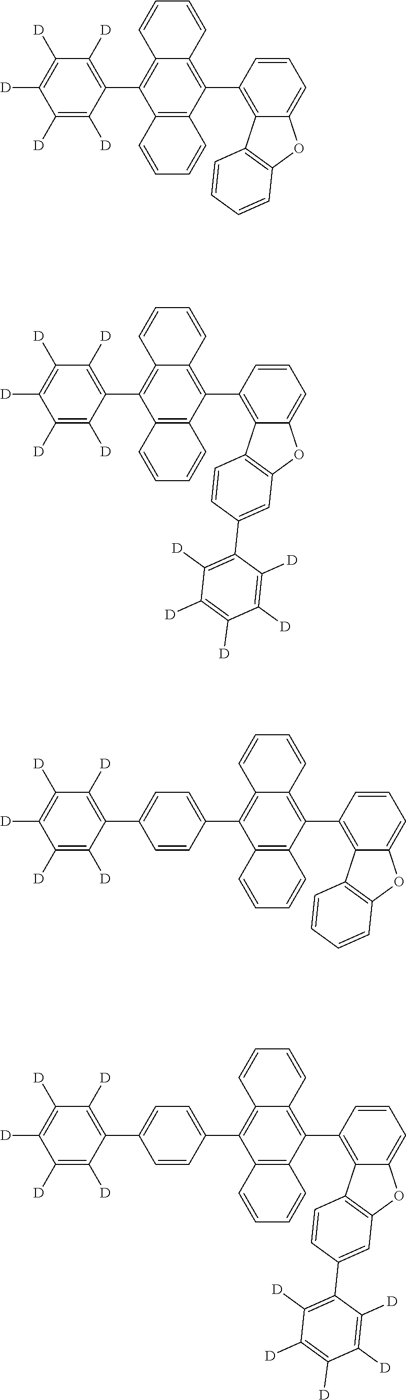 [SMILES: [2H]c1c([2H])c([2H])c(-c2c3ccccc3c(-c3cccc4oc5ccccc5c34)c3ccccc23)c([2H])c1[2H] ; [2H]c1c([2H])c([2H])c(-c2ccc3c(c2)oc2cccc(-c4c5ccccc5c(-c5c([2H])c([2H])c([2H])c([2H])c5[2H])c5ccccc45)c23)c([2H])c1[2H] ; [2H]c1c([2H])c([2H])c(-c2ccc(-c3c4ccccc4c(-c4cccc5oc6ccccc6c45)c4ccccc34)cc2)c([2H])c1[2H] ; [2H]c1c([2H])c([2H])c(-c2ccc(-c3c4ccccc4c(-c4cccc5oc6cc(-c7c([2H])c([2H])c([2H])c([2H])c7[2H])ccc6c45)c4ccccc34)cc2)c([2H])c1[2H]]

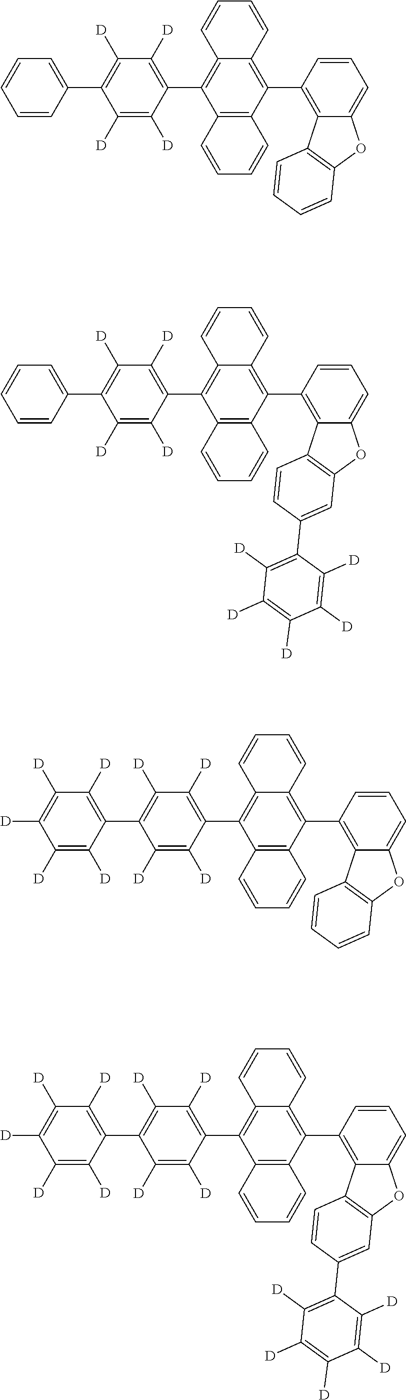 [SMILES: [2H]c1c([2H])c(-c2c3ccccc3c(-c3cccc4oc5ccccc5c34)c3ccccc23)c([2H])c([2H])c1-c1ccccc1 ; [2H]c1c([2H])c([2H])c(-c2ccc3c(c2)oc2cccc(-c4c5ccccc5c(-c5c([2H])c([2H])c(-c6ccccc6)c([2H])c5[2H])c5ccccc45)c23)c([2H])c1[2H] ; [2H]c1c([2H])c([2H])c(-c2c([2H])c([2H])c(-c3c4ccccc4c(-c4cccc5oc6ccccc6c45)c4ccccc34)c([2H])c2[2H])c([2H])c1[2H] ; [2H]c1c([2H])c([2H])c(-c2ccc3c(c2)oc2cccc(-c4c5ccccc5c(-c5c([2H])c([2H])c(-c6c([2H])c([2H])c([2H])c([2H])c6[2H])c([2H])c5[2H])c5ccccc45)c23)c([2H])c1[2H]]

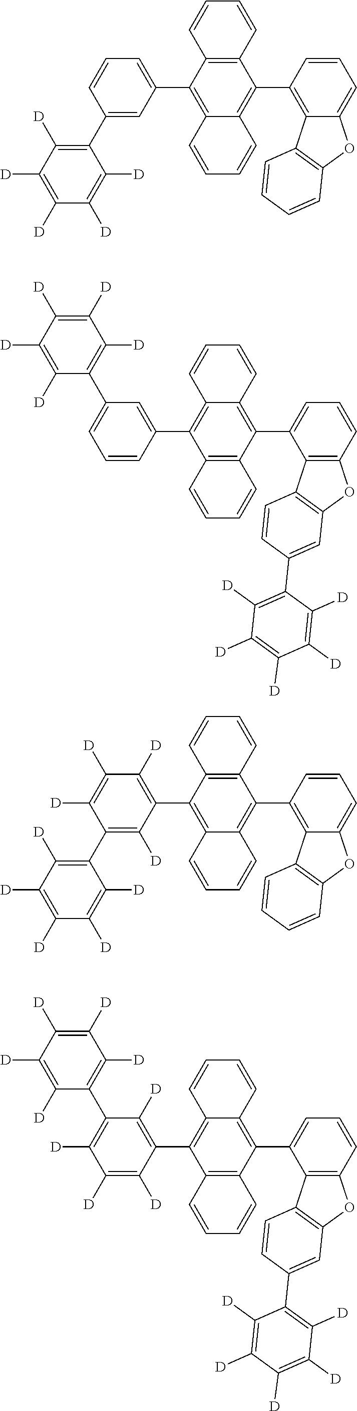

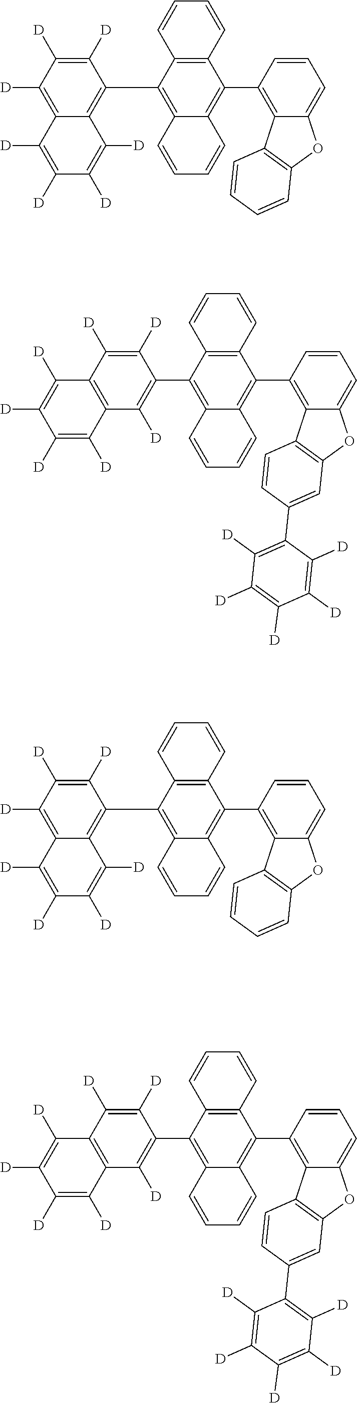 [SMILES: [2H]c1c([2H])c([2H])c2c(-c3c4ccccc4c(-c4cccc5oc6ccccc6c45)c4ccccc34)c([2H])c([2H])c([2H])c2c1[2H] ; [2H]c1c([2H])c([2H])c(-c2ccc3c(c2)oc2cccc(-c4c5ccccc5c(-c5c([2H])c([2H])c6c([2H])c([2H])c([2H])c([2H])c6c5[2H])c5ccccc45)c23)c([2H])c1[2H] ; [2H]c1c([2H])c([2H])c2c(-c3c4ccccc4c(-c4cccc5oc6ccccc6c45)c4ccccc34)c([2H])c([2H])c([2H])c2c1[2H] ; [2H]c1c([2H])c([2H])c(-c2ccc3c(c2)oc2cccc(-c4c5ccccc5c(-c5c([2H])c([2H])c6c([2H])c([2H])c([2H])c([2H])c6c5[2H])c5ccccc45)c23)c([2H])c1[2H]]

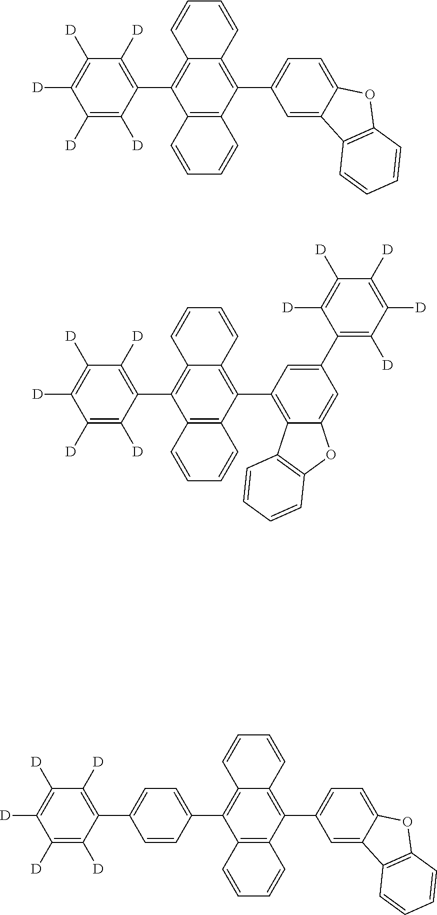 [SMILES: [2H]c1c([2H])c([2H])c(-c2c3ccccc3c(-c3ccc4oc5ccccc5c4c3)c3ccccc23)c([2H])c1[2H] ; [2H]c1c([2H])c([2H])c(-c2cc(-c3c4ccccc4c(-c4c([2H])c([2H])c([2H])c([2H])c4[2H])c4ccccc34)c3c(c2)oc2ccccc23)c([2H])c1[2H] ; [2H]c1c([2H])c([2H])c(-c2ccc(-c3c4ccccc4c(-c4ccc5oc6ccccc6c5c4)c4ccccc34)cc2)c([2H])c1[2H]]

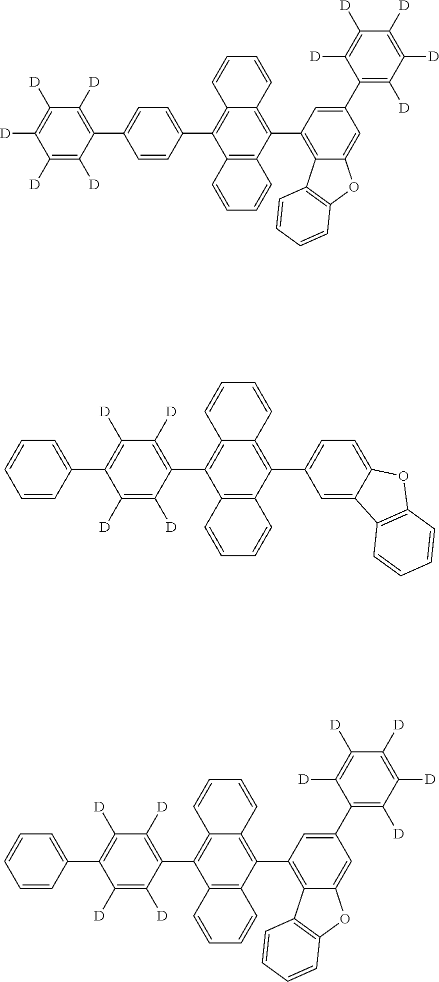 [SMILES: [2H]c1c([2H])c([2H])c(-c2ccc(-c3c4ccccc4c(-c4cc(-c5c([2H])c([2H])c([2H])c([2H])c5[2H])cc5oc6ccccc6c45)c4ccccc34)cc2)c([2H])c1[2H] ; [2H]c1c([2H])c(-c2c3ccccc3c(-c3ccc4oc5ccccc5c4c3)c3ccccc23)c([2H])c([2H])c1-c1ccccc1 ; [2H]c1c([2H])c([2H])c(-c2cc(-c3c4ccccc4c(-c4c([2H])c([2H])c(-c5ccccc5)c([2H])c4[2H])c4ccccc34)c3c(c2)oc2ccccc23)c([2H])c1[2H]]

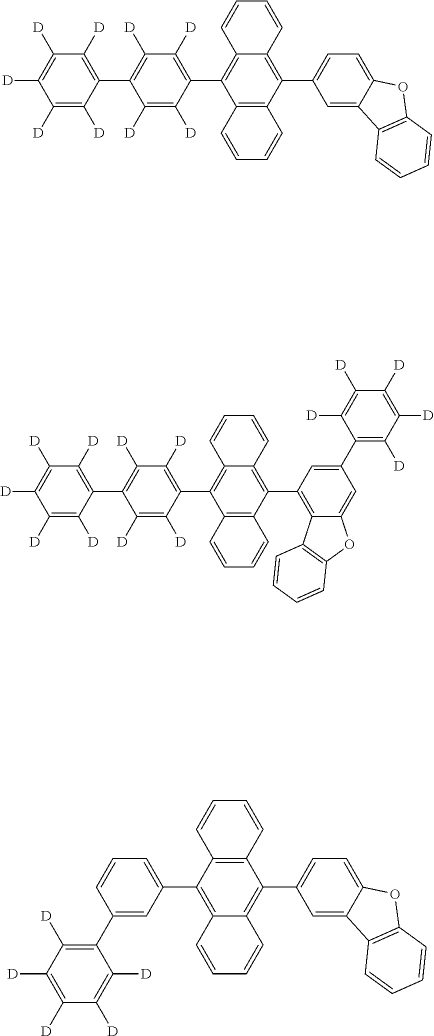 [SMILES: [2H]c1c([2H])c([2H])c(-c2c([2H])c([2H])c(-c3c4ccccc4c(-c4ccc5oc6ccccc6c5c4)c4ccccc34)c([2H])c2[2H])c([2H])c1[2H] ; [2H]c1c([2H])c([2H])c(-c2cc(-c3c4ccccc4c(-c4c([2H])c([2H])c(-c5c([2H])c([2H])c([2H])c([2H])c5[2H])c([2H])c4[2H])c4ccccc34)c3c(c2)oc2ccccc23)c([2H])c1[2H] ; [2H]c1c([2H])c([2H])c(-c2cccc(-c3c4ccccc4c(-c4ccc5oc6ccccc6c5c4)c4ccccc34)c2)c([2H])c1[2H]]

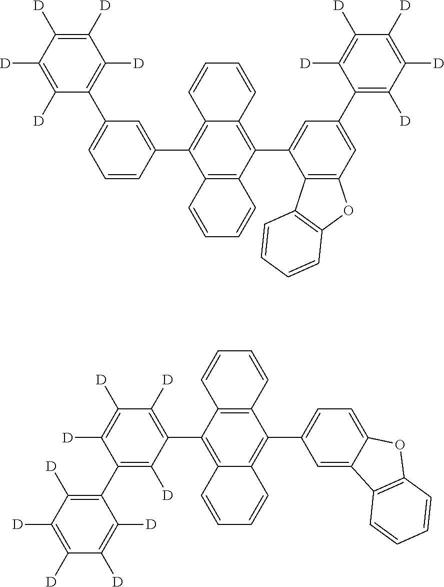 [SMILES: [2H]c1c([2H])c([2H])c(-c2cccc(-c3c4ccccc4c(-c4cc(-c5c([2H])c([2H])c([2H])c([2H])c5[2H])cc5oc6ccccc6c45)c4ccccc34)c2)c([2H])c1[2H] ; [2H]c1c([2H])c([2H])c(-c2c([2H])c([2H])c([2H])c(-c3c4ccccc4c(-c4ccc5oc6ccccc6c5c4)c4ccccc34)c2[2H])c([2H])c1[2H]]

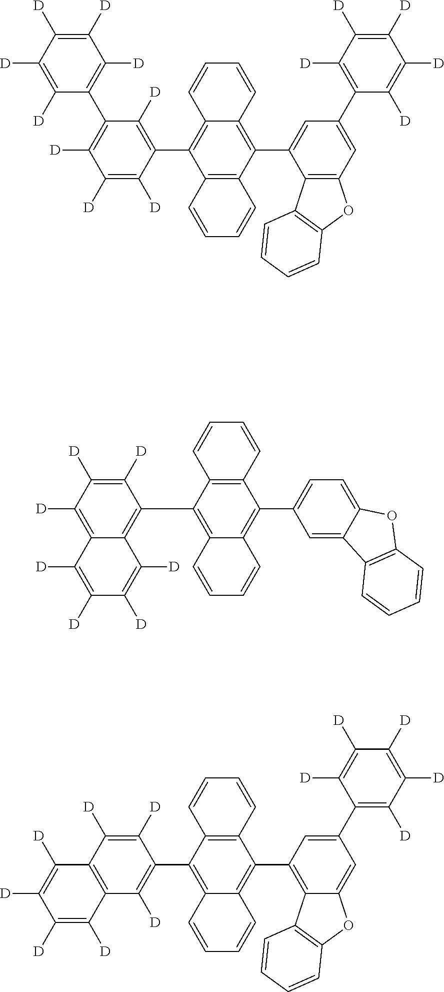 [SMILES: [2H]c1c([2H])c([2H])c(-c2cc(-c3c4ccccc4c(-c4c([2H])c([2H])c([2H])c(-c5c([2H])c([2H])c([2H])c([2H])c5[2H])c4[2H])c4ccccc34)c3c(c2)oc2ccccc23)c([2H])c1[2H] ; [2H]c1c([2H])c([2H])c2c(-c3c4ccccc4c(-c4ccc5oc6ccccc6c5c4)c4ccccc34)c([2H])c([2H])c([2H])c2c1[2H] ; [2H]c1c([2H])c([2H])c(-c2cc(-c3c4ccccc4c(-c4c([2H])c([2H])c5c([2H])c([2H])c([2H])c([2H])c5c4[2H])c4ccccc34)c3c(c2)oc2ccccc23)c([2H])c1[2H]]

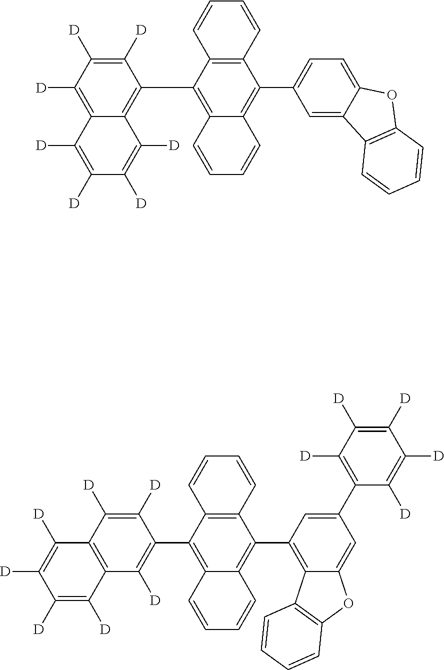 [SMILES: [2H]c1c([2H])c([2H])c2c(-c3c4ccccc4c(-c4ccc5oc6ccccc6c5c4)c4ccccc34)c([2H])c([2H])c([2H])c2c1[2H] ; [2H]c1c([2H])c([2H])c(-c2cc(-c3c4ccccc4c(-c4c([2H])c([2H])c5c([2H])c([2H])c([2H])c([2H])c5c4[2H])c4ccccc34)c3c(c2)oc2ccccc23)c([2H])c1[2H]]

### Emitting Compound

In the organic EL device according to the third exemplary embodiment, emitting compounds such as the first emitting compound and the second emitting compound are not particularly limited. For instance, the emitting compounds are also preferably each independently at least one compound selected from the group consisting of a compound represented by a formula (4) below, a compound represented by a formula (5) below, and a compound represented by a formula (6) below.

### Compound Represented by Formula (4)

The compound represented by the formula (4) will be described below.

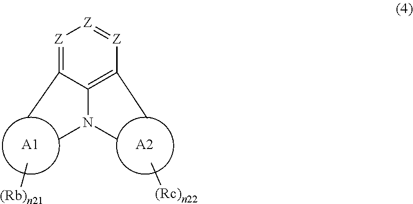

In the formula (4):
Z are each independently CRa or a nitrogen atom;
A1 ring and A2 ring are each independently a substituted or unsubstituted aromatic hydrocarbon ring having 6 to 50 ring carbon atoms, or a substituted or unsubstituted heterocycle having 5 to 50 ring atoms;
when a plurality of Ra are present, at least one combination of adjacent two or more of the plurality of Ra are mutually bonded to form a substituted or unsubstituted monocyclic ring, mutually bonded to form a substituted or unsubstituted fused ring, or not mutually bonded; and
n21 and n22 are each independently 0, 1, 2, 3 or 4;
when a plurality of Rb are present, at least one combination of adjacent two or more of the plurality of Rb are mutually bonded to form a substituted or unsubstituted monocyclic ring, mutually bonded to form a substituted or unsubstituted fused ring, or not mutually bonded; and
when a plurality of Rc are present, at least one combination of adjacent two or more of the plurality of Rc are mutually bonded to form a substituted or unsubstituted monocyclic ring, mutually bonded to form a substituted or unsubstituted fused ring, or not mutually bonded; and
Ra, Rb, and Rc forming neither the substituted or unsubstituted monocyclic ring nor the substituted or unsubstituted fused ring are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

### Specific Examples of Compound Represented by Formula (4)

Specific examples of the compound represented by the formula (4) include compounds shown below.

In specific examples of compounds herein, occasionaly, Ph represents a phenyl group, Me represents a methyl group, D represents a deuterium atom, tBu represents a tert-butyl group, and tAm represents a tert-amyl group.

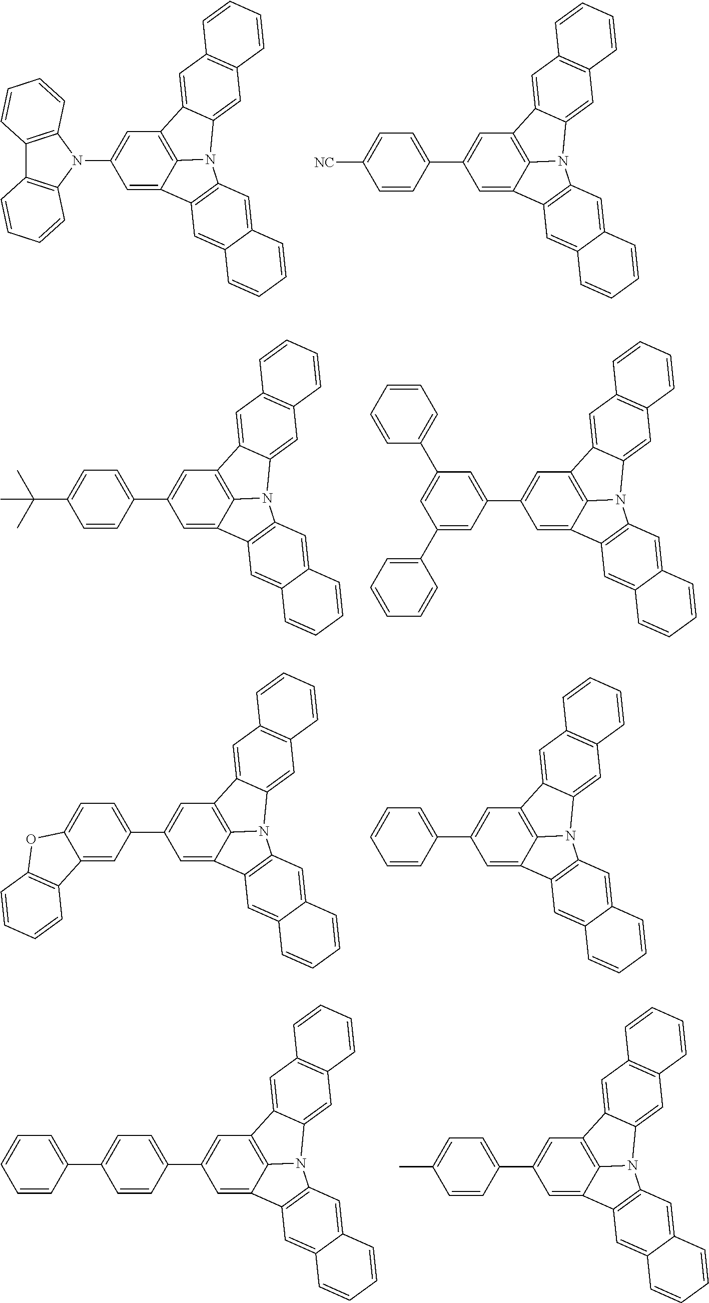

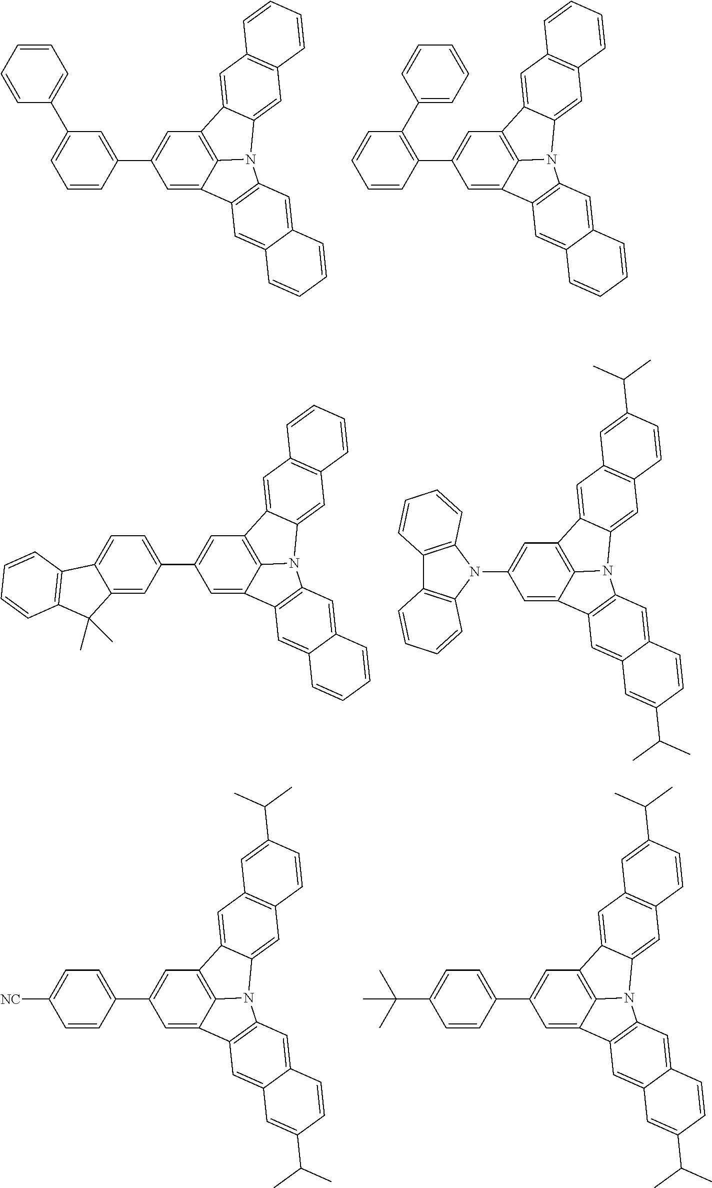

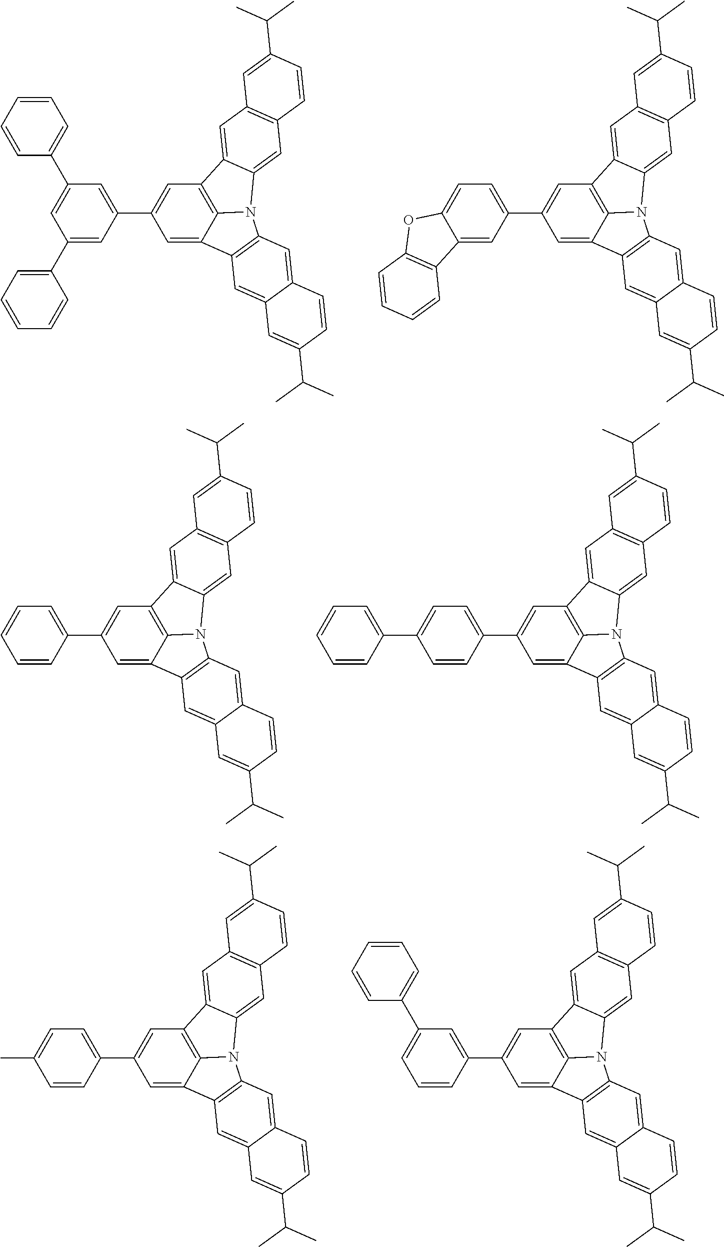

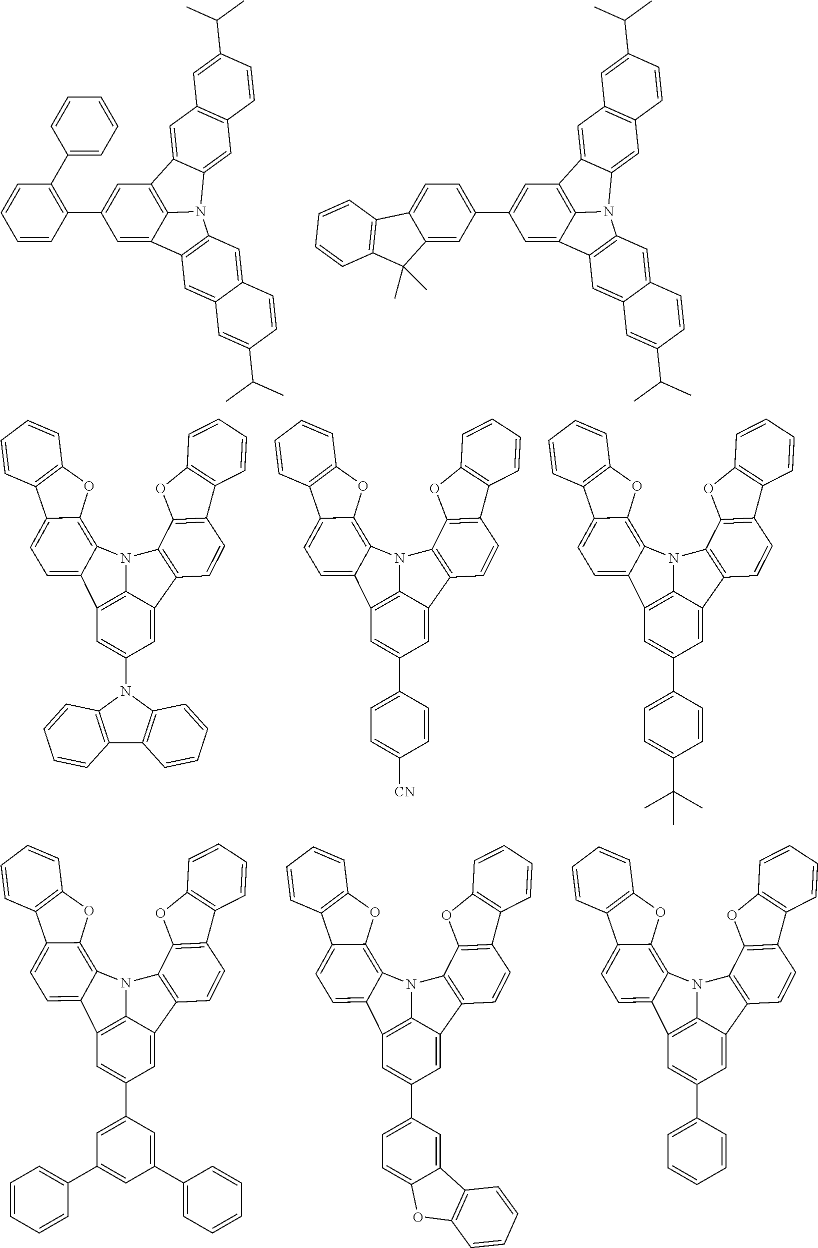

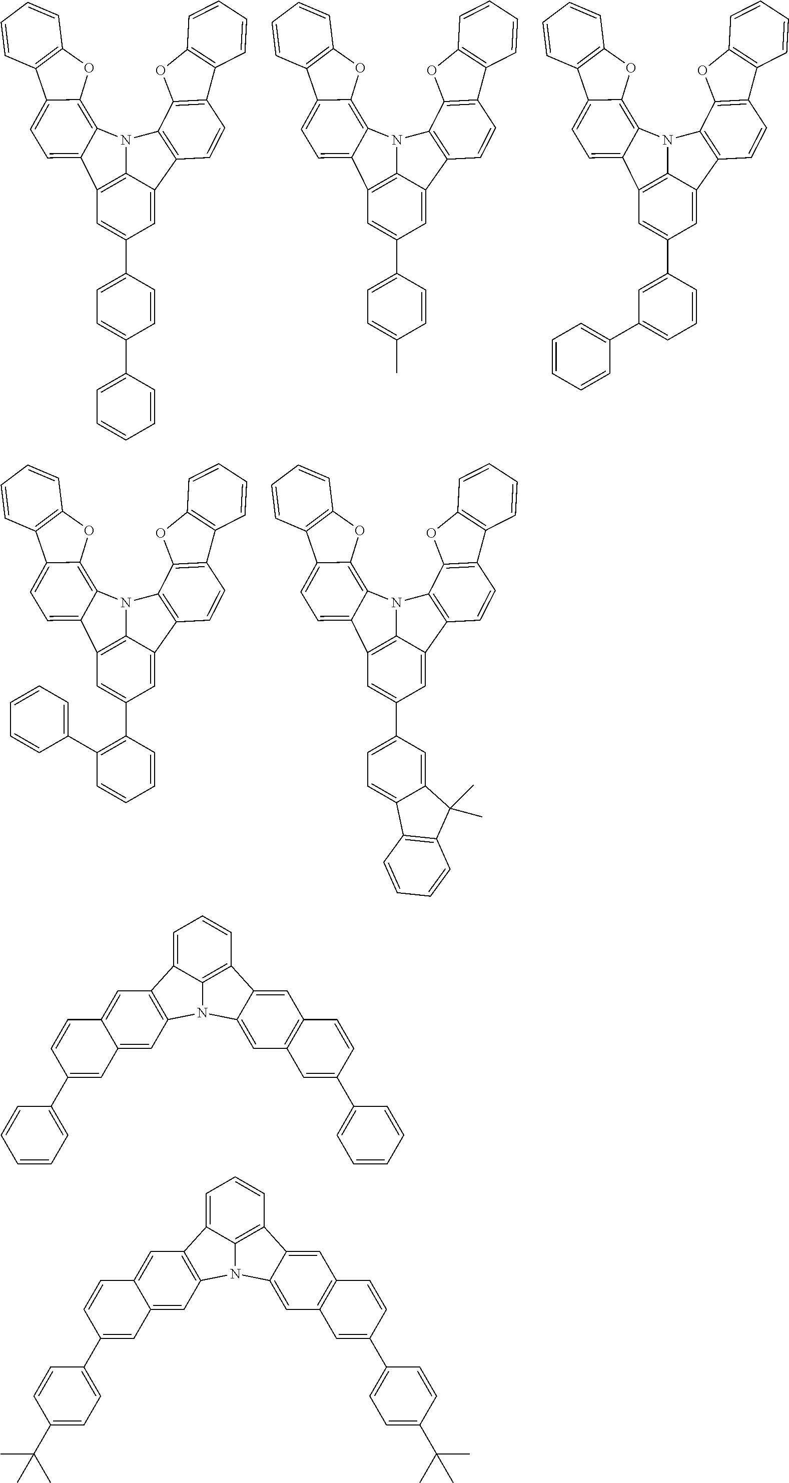

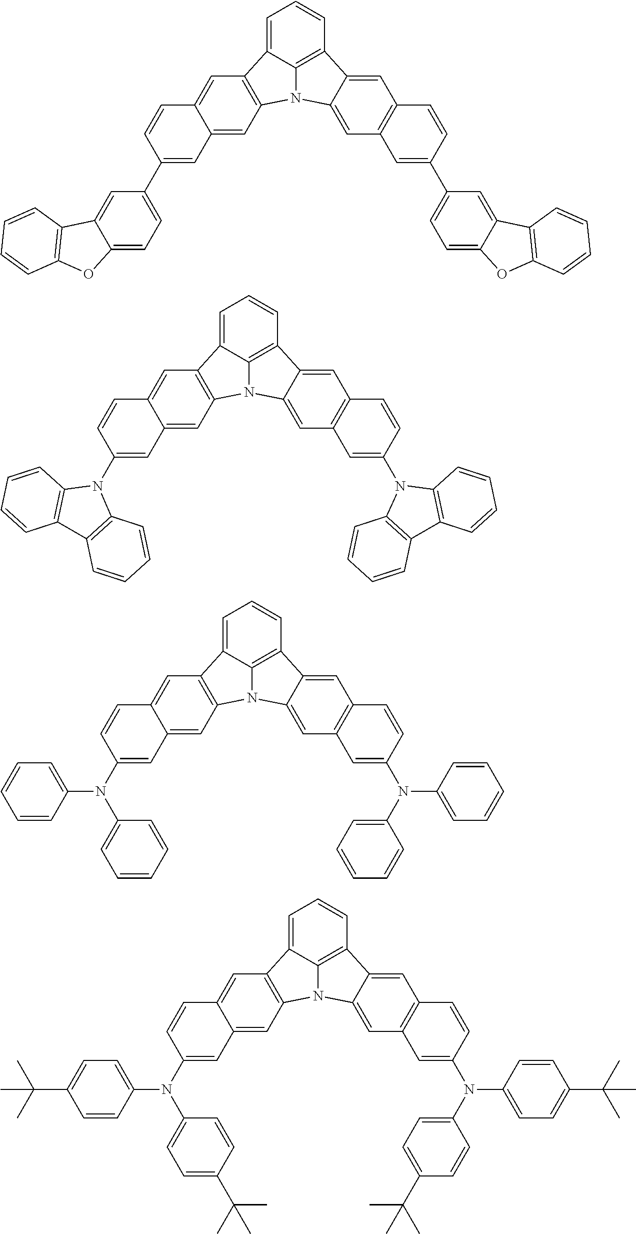

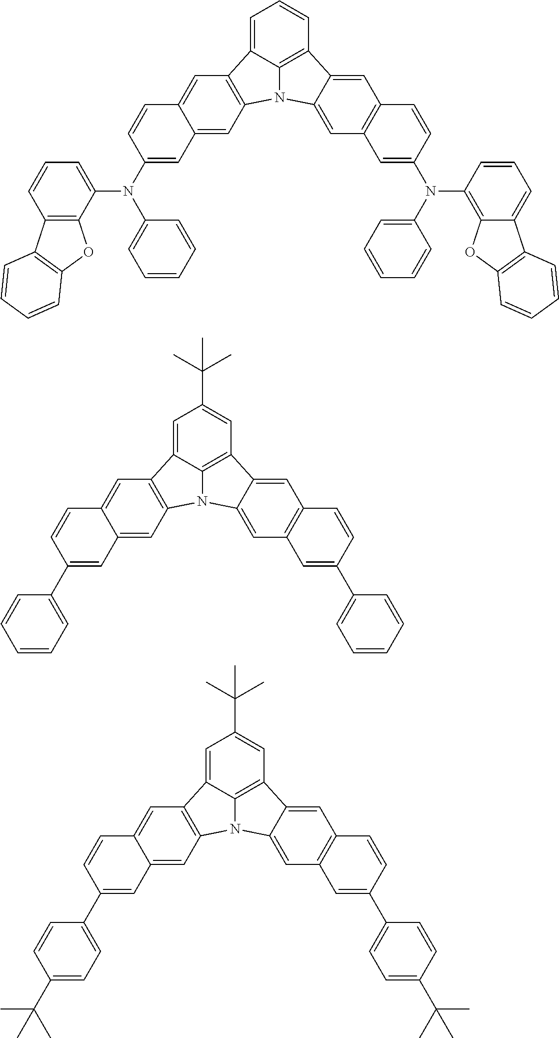 [SMILES: c1ccc(N(c2ccc3cc4c5cccc6c7cc8ccc(N(c9ccccc9)c9cccc%10c9oc9ccccc9%10)cc8cc7n(c4cc3c2)c56)c2cccc3c2oc2ccccc23)cc1 ; CC(C)(C)c1cc2c3cc4ccc(-c5ccccc5)cc4cc3n3c4cc5cc(-c6ccccc6)ccc5cc4c(c1)c23 ; CC(C)(C)c1ccc(-c2ccc3cc4c5cc(C(C)(C)C)cc6c7cc8ccc(-c9ccc(C(C)(C)C)cc9)cc8cc7n(c4cc3c2)c56)cc1]

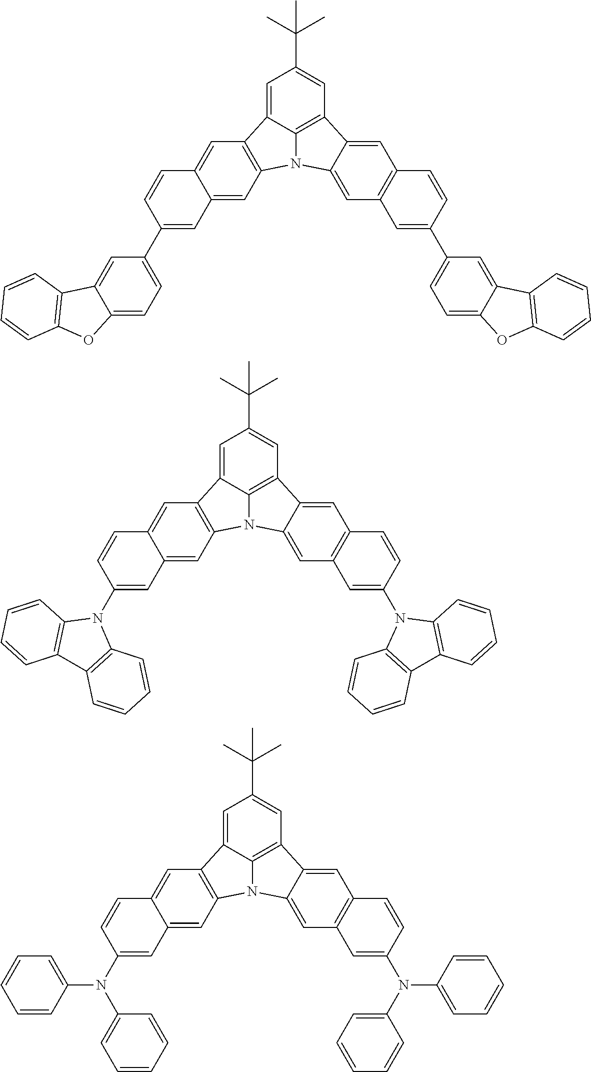 [SMILES: CC(C)(C)c1cc2c3cc4ccc(-c5ccc6oc7ccccc7c6c5)cc4cc3n3c4cc5cc(-c6ccc7oc8ccccc8c7c6)ccc5cc4c(c1)c23 ; CC(C)(C)c1cc2c3cc4ccc(-n5c6ccccc6c6ccccc65)cc4cc3n3c4cc5cc(-n6c7ccccc7c7ccccc76)ccc5cc4c(c1)c23 ; CC(C)(C)c1cc2c3cc4ccc(N(c5ccccc5)c5ccccc5)cc4cc3n3c4cc5cc(N(c6ccccc6)c6ccccc6)ccc5cc4c(c1)c23]

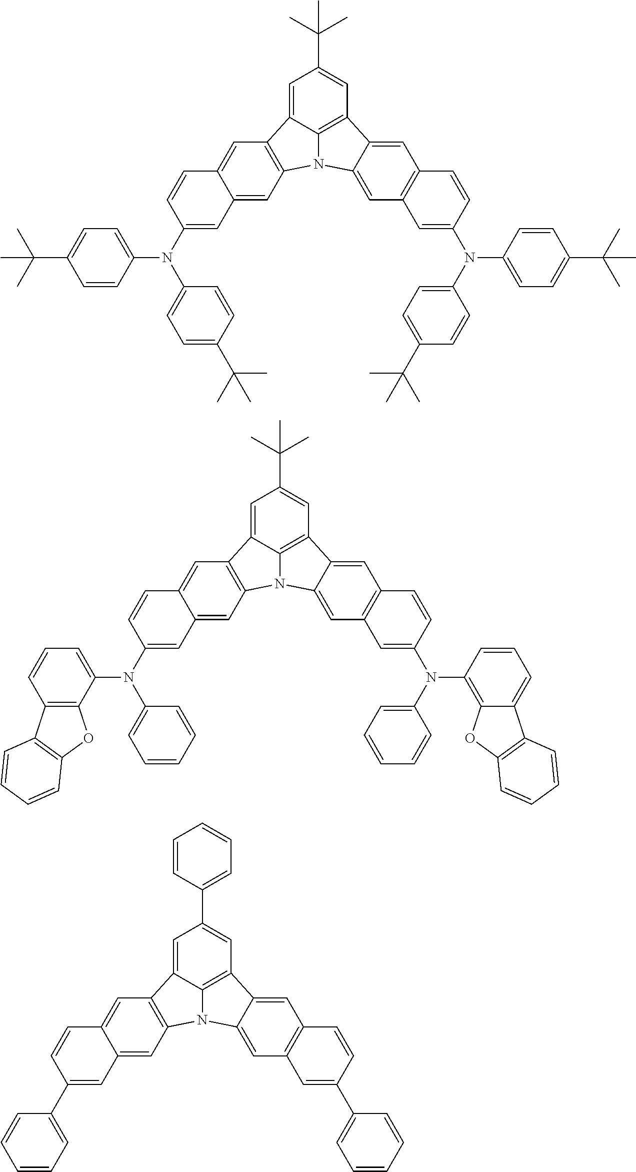

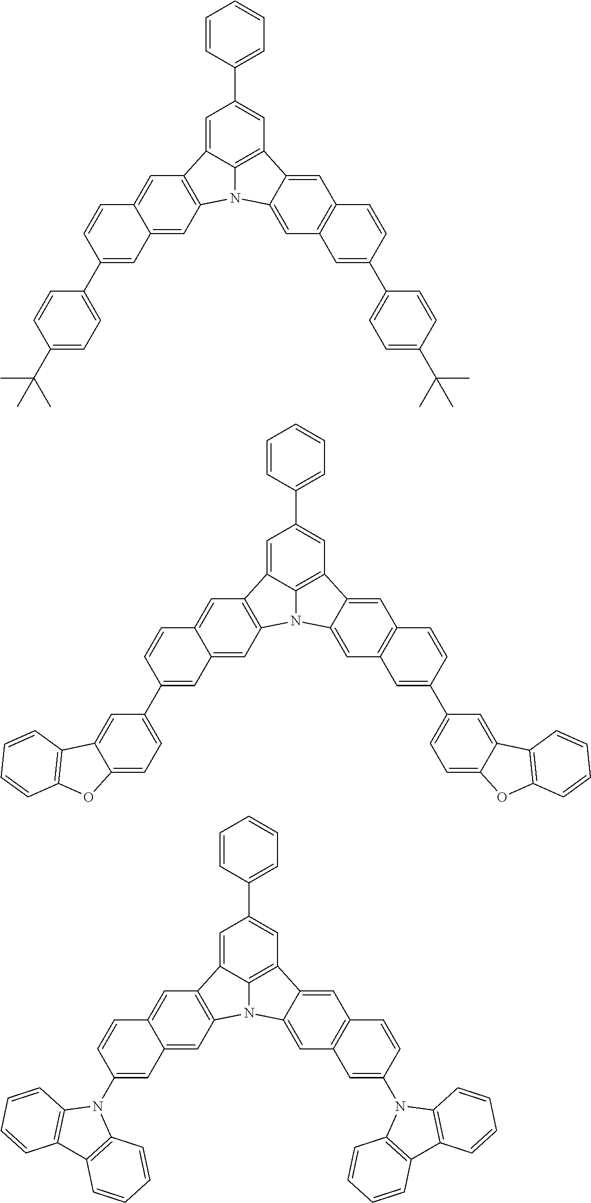 [SMILES: CC(C)(C)c1ccc(-c2ccc3cc4c5cc(-c6ccccc6)cc6c7cc8ccc(-c9ccc(C(C)(C)C)cc9)cc8cc7n(c4cc3c2)c56)cc1 ; c1ccc(-c2cc3c4cc5ccc(-c6ccc7oc8ccccc8c7c6)cc5cc4n4c5cc6cc(-c7ccc8oc9ccccc9c8c7)ccc6cc5c(c2)c34)cc1 ; c1ccc(-c2cc3c4cc5ccc(-n6c7ccccc7c7ccccc76)cc5cc4n4c5cc6cc(-n7c8ccccc8c8ccccc87)ccc6cc5c(c2)c34)cc1]

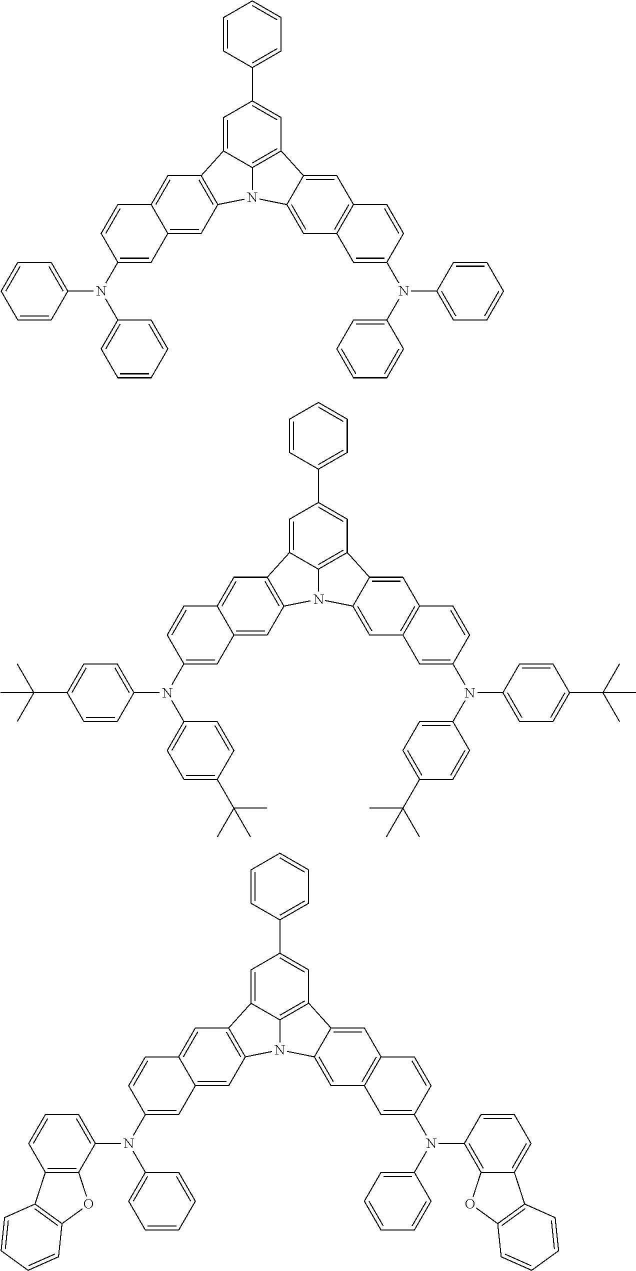

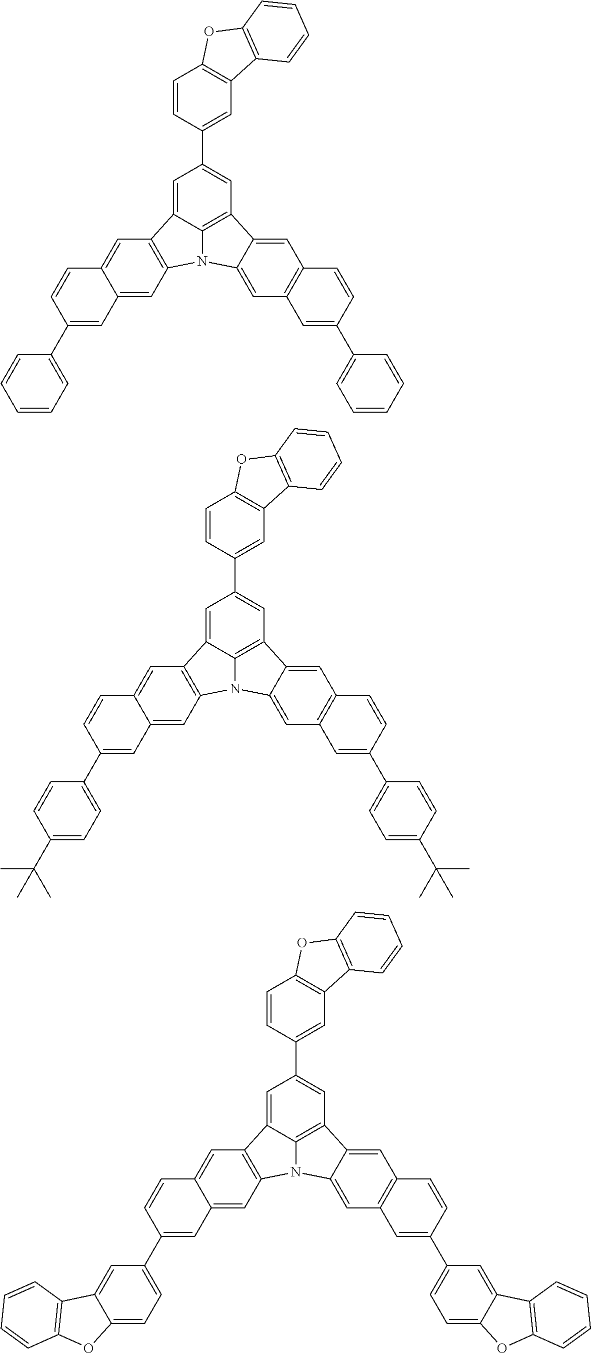

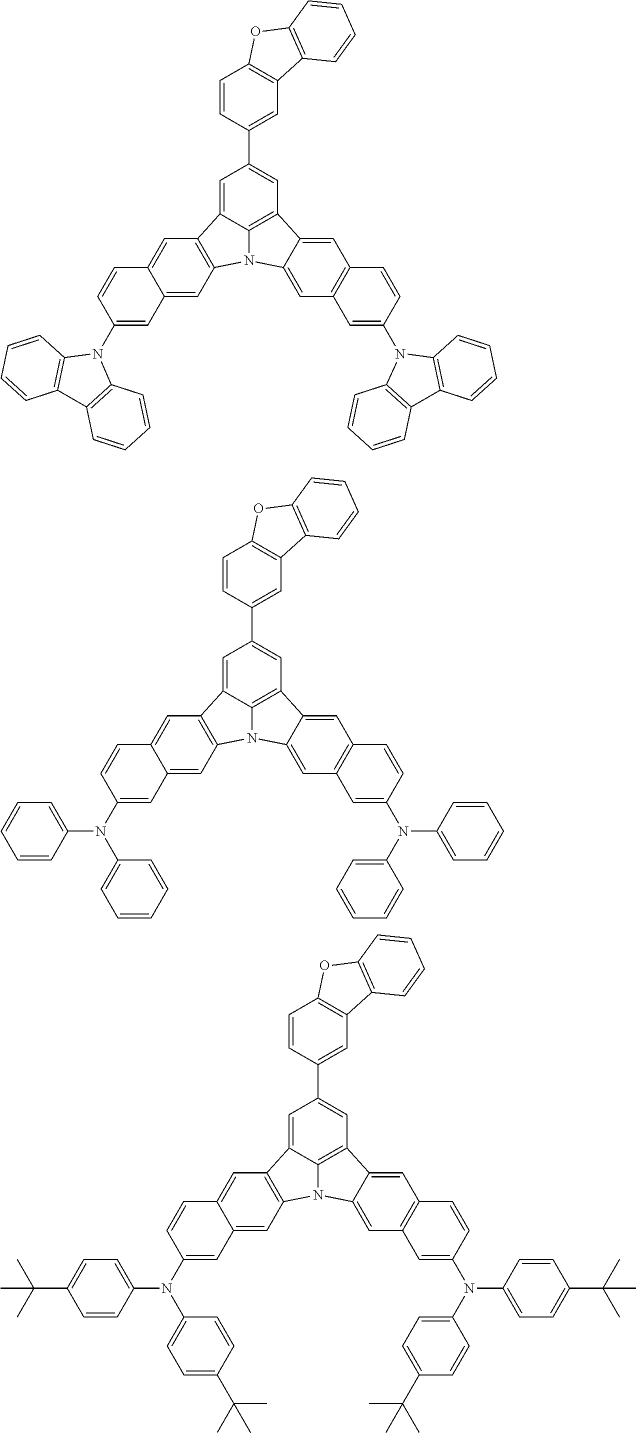

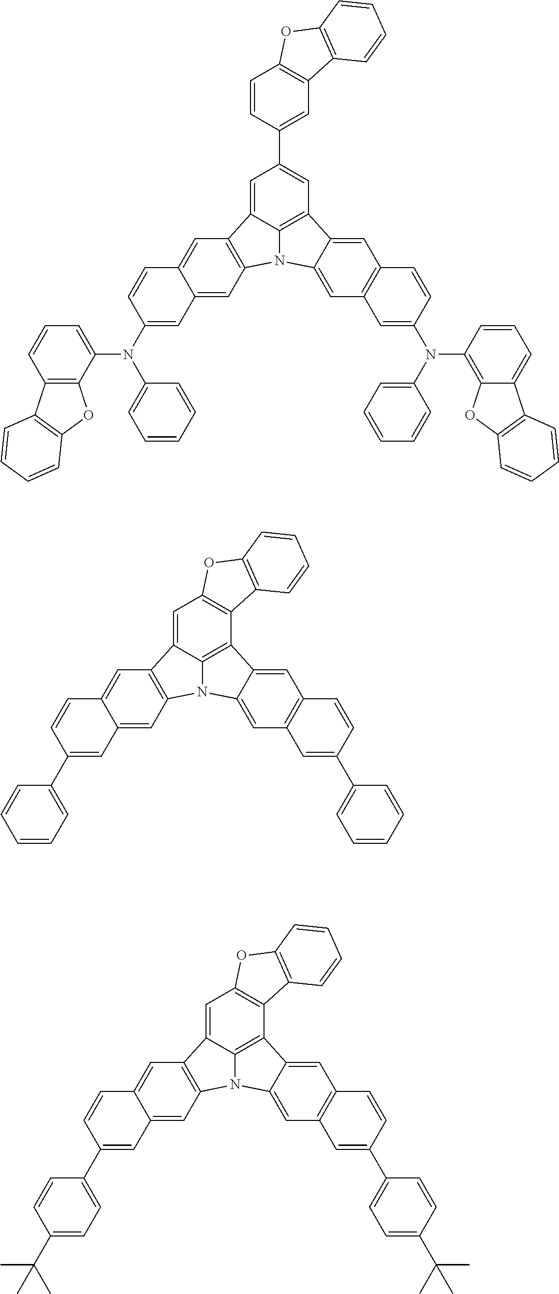 [SMILES: c1ccc(N(c2ccc3cc4c5cc(-c6ccc7oc8ccccc8c7c6)cc6c7cc8ccc(N(c9ccccc9)c9cccc%10c9oc9ccccc9%10)cc8cc7n(c4cc3c2)c56)c2cccc3c2oc2ccccc23)cc1 ; c1ccc(-c2ccc3cc4c5cc6oc7ccccc7c6c6c7cc8ccc(-c9ccccc9)cc8cc7n(c4cc3c2)c56)cc1 ; CC(C)(C)c1ccc(-c2ccc3cc4c5cc6oc7ccccc7c6c6c7cc8ccc(-c9ccc(C(C)(C)C)cc9)cc8cc7n(c4cc3c2)c56)cc1]

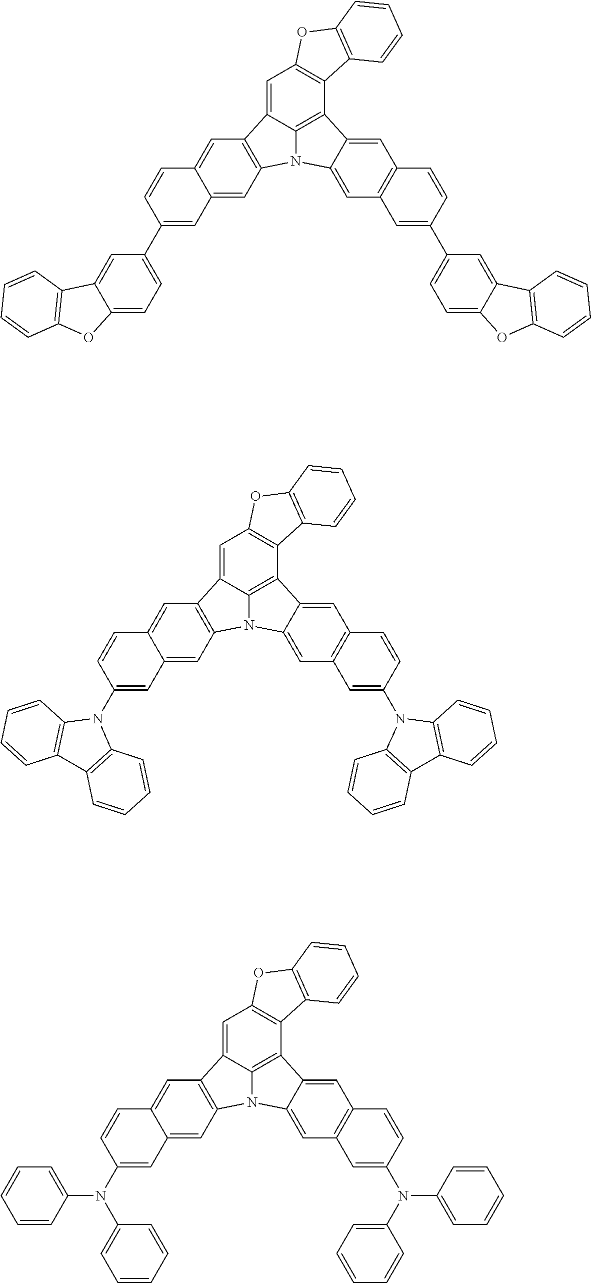 [SMILES: c1ccc2c(c1)oc1ccc(-c3ccc4cc5c6cc7oc8ccccc8c7c7c8cc9ccc(-c%10ccc%11oc%12ccccc%12c%11c%10)cc9cc8n(c5cc4c3)c67)cc12 ; c1ccc2c(c1)oc1cc3c4cc5ccc(-n6c7ccccc7c7ccccc76)cc5cc4n4c5cc6cc(-n7c8ccccc8c8ccccc87)ccc6cc5c(c12)c34 ; c1ccc(N(c2ccccc2)c2ccc3cc4c5cc6oc7ccccc7c6c6c7cc8ccc(N(c9ccccc9)c9ccccc9)cc8cc7n(c4cc3c2)c56)cc1]

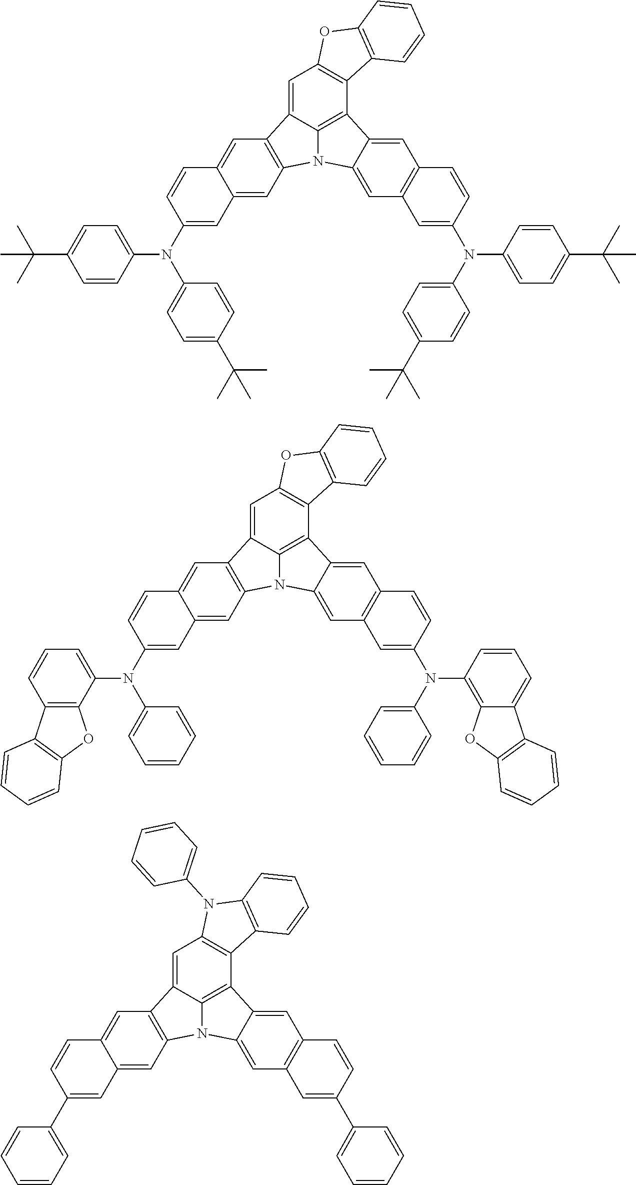

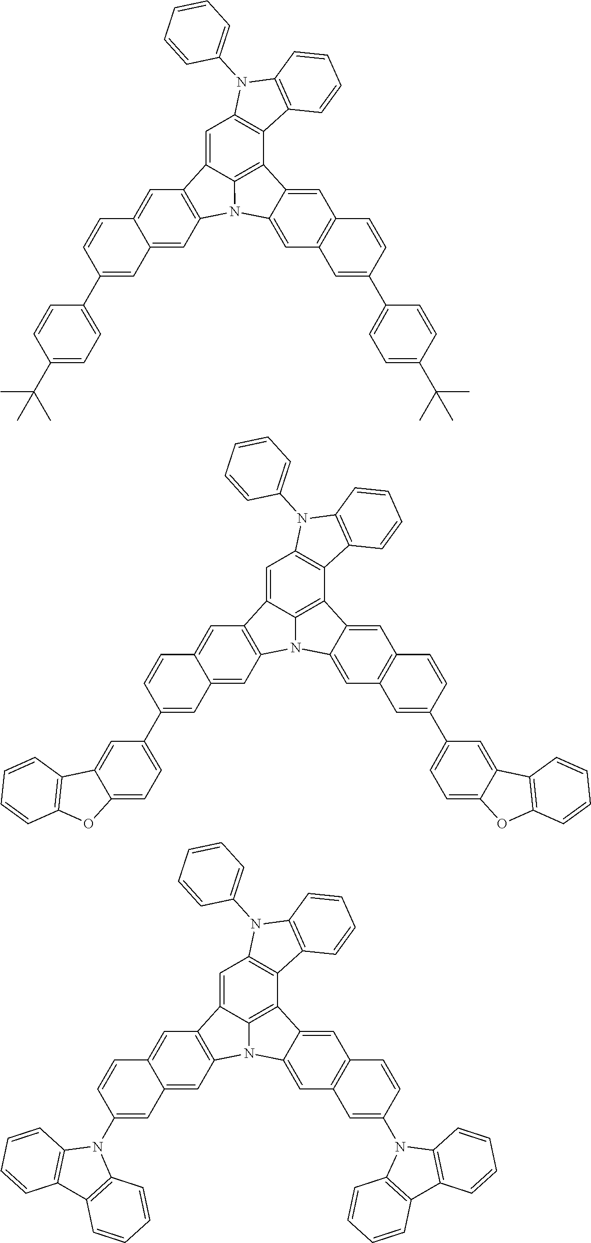 [SMILES: CC(C)(C)c1ccc(-c2ccc3cc4c5cc6c(c7ccccc7n6-c6ccccc6)c6c7cc8ccc(-c9ccc(C(C)(C)C)cc9)cc8cc7n(c4cc3c2)c56)cc1 ; c1ccc(-n2c3ccccc3c3c4c5cc6ccc(-c7ccc8oc9ccccc9c8c7)cc6cc5n5c6cc7cc(-c8ccc9oc%10ccccc%10c9c8)ccc7cc6c(cc32)c45)cc1 ; c1ccc(-n2c3ccccc3c3c4c5cc6ccc(-n7c8ccccc8c8ccccc87)cc6cc5n5c6cc7cc(-n8c9ccccc9c9ccccc98)ccc7cc6c(cc32)c45)cc1]

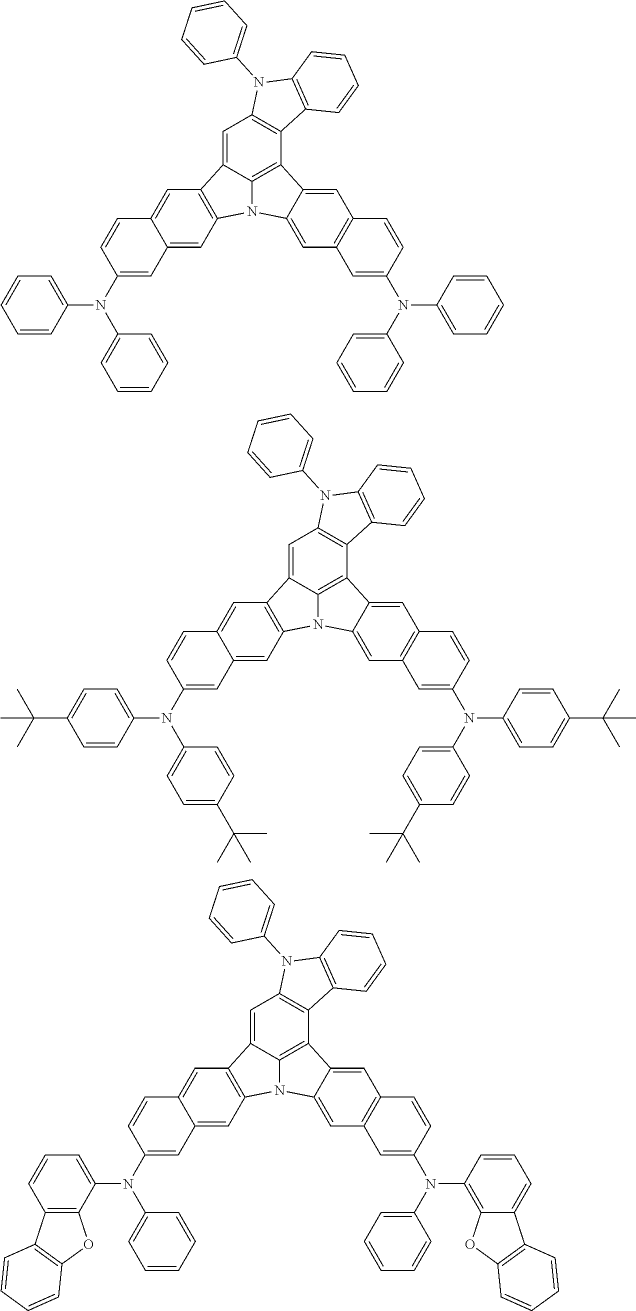

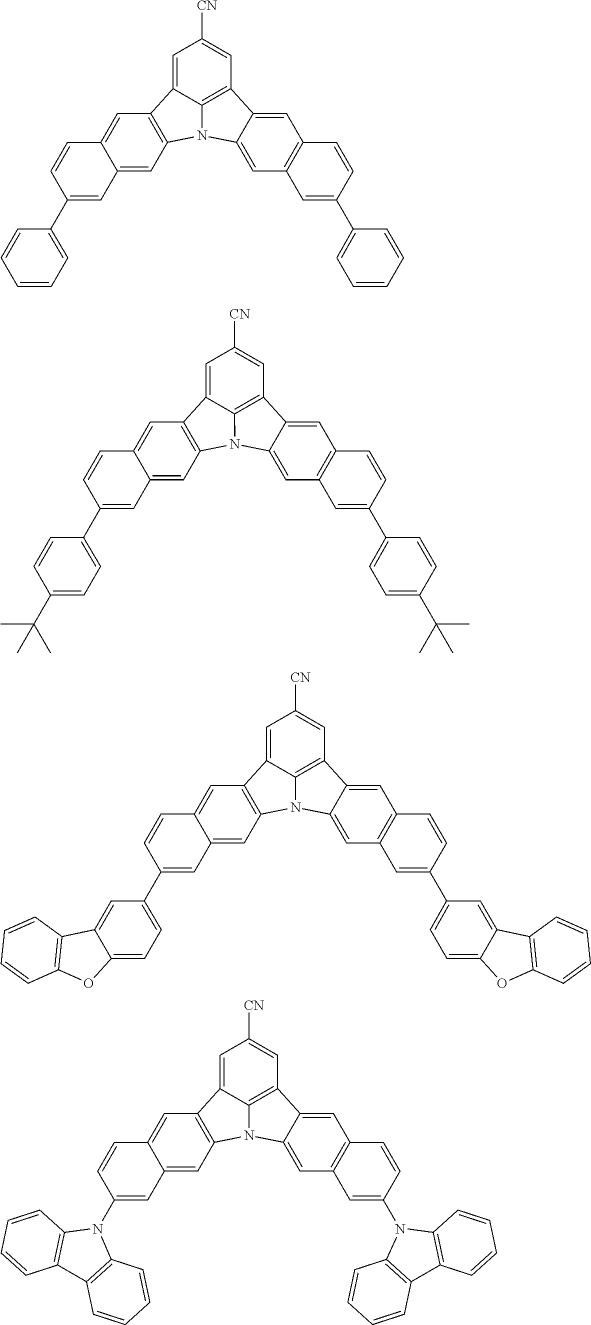 [SMILES: N#Cc1cc2c3cc4ccc(-c5ccccc5)cc4cc3n3c4cc5cc(-c6ccccc6)ccc5cc4c(c1)c23 ; CC(C)(C)c1ccc(-c2ccc3cc4c5cc(C#N)cc6c7cc8ccc(-c9ccc(C(C)(C)C)cc9)cc8cc7n(c4cc3c2)c56)cc1 ; N#Cc1cc2c3cc4ccc(-c5ccc6oc7ccccc7c6c5)cc4cc3n3c4cc5cc(-c6ccc7oc8ccccc8c7c6)ccc5cc4c(c1)c23 ; N#Cc1cc2c3cc4ccc(-n5c6ccccc6c6ccccc65)cc4cc3n3c4cc5cc(-n6c7ccccc7c7ccccc76)ccc5cc4c(c1)c23]

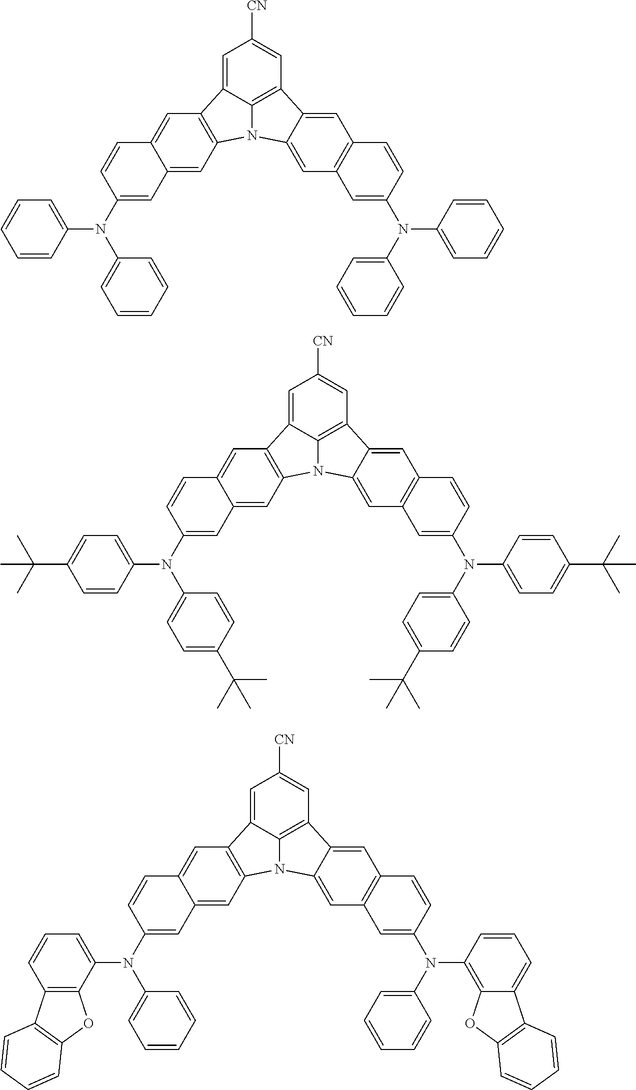

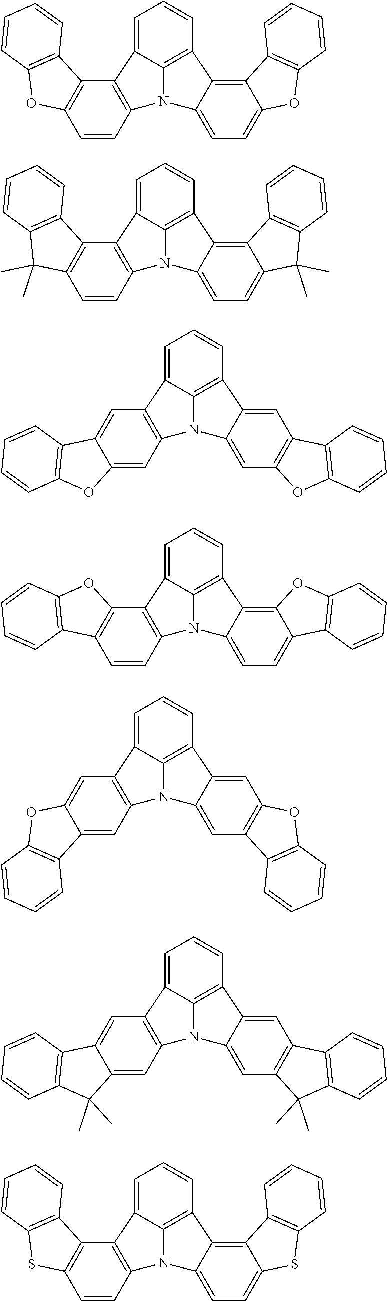

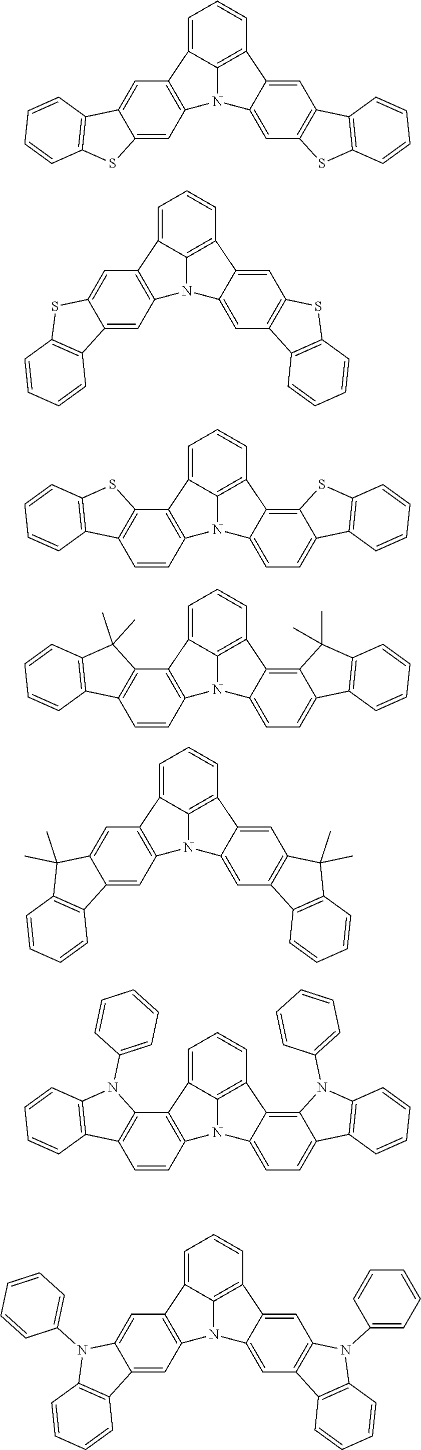

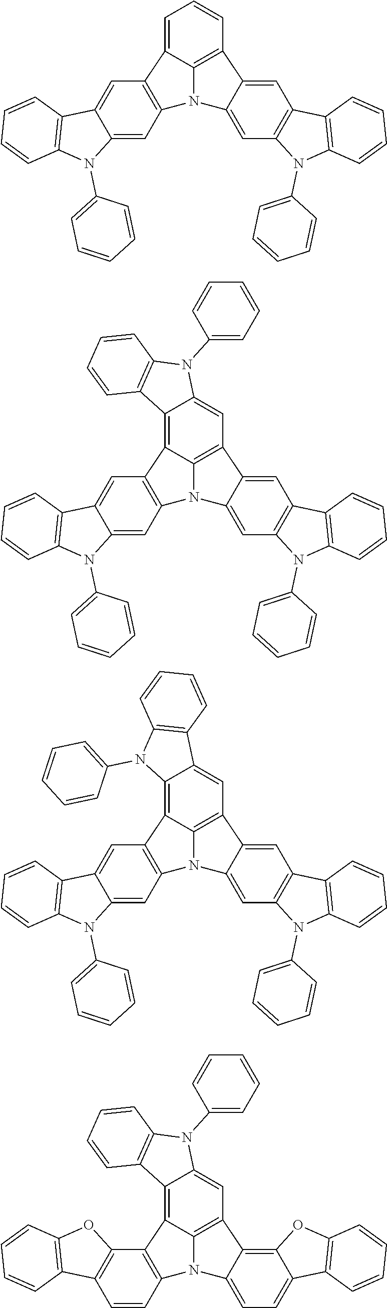

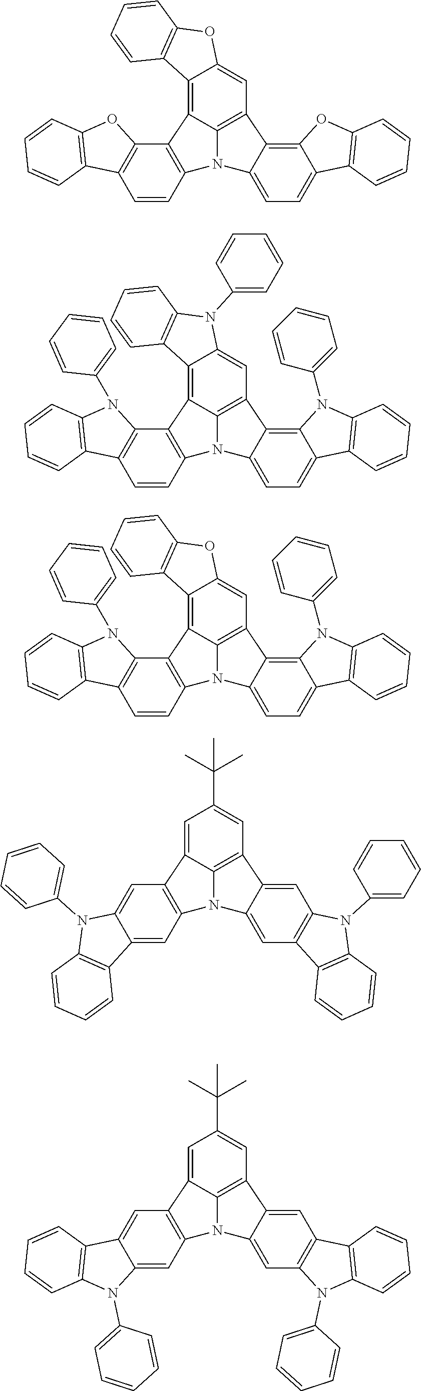

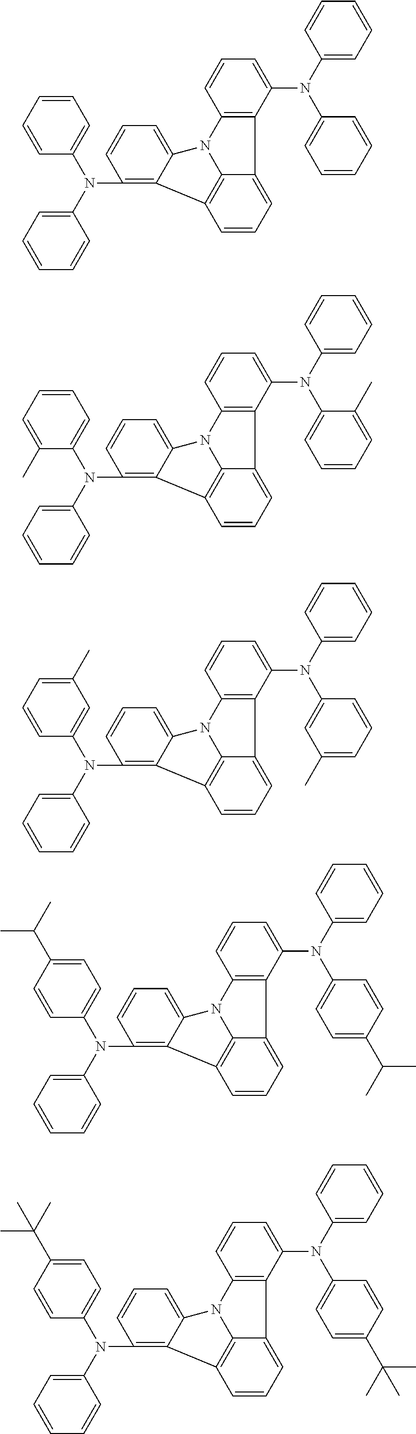

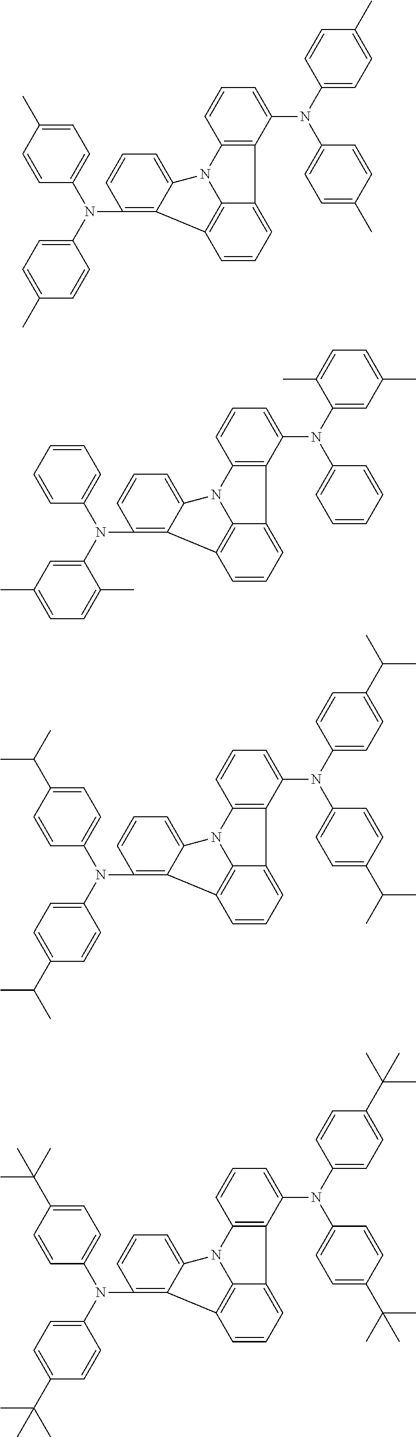

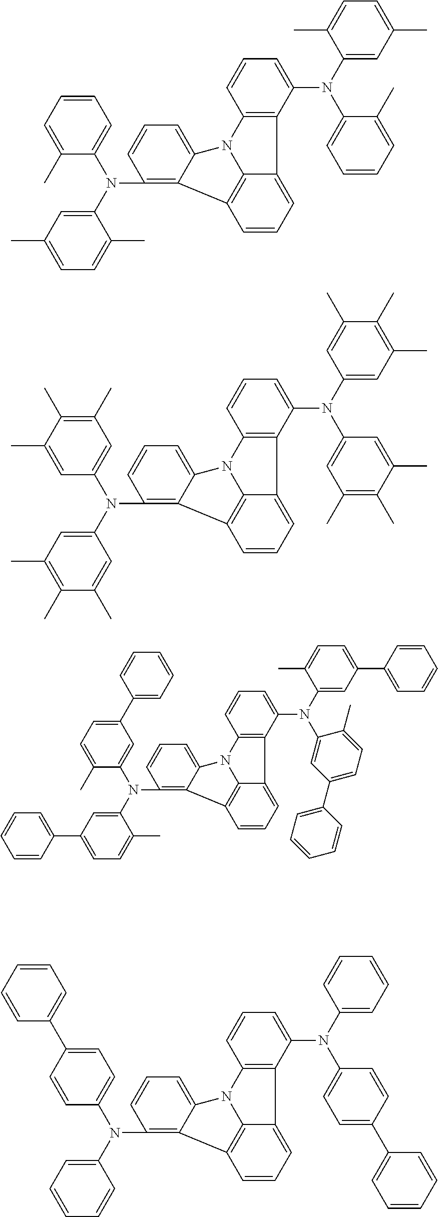

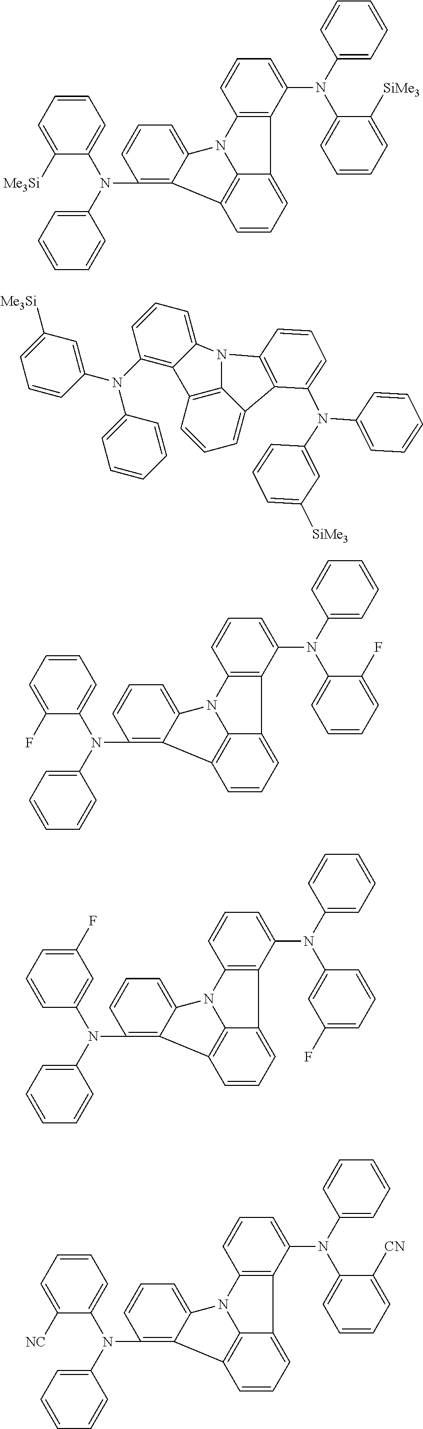

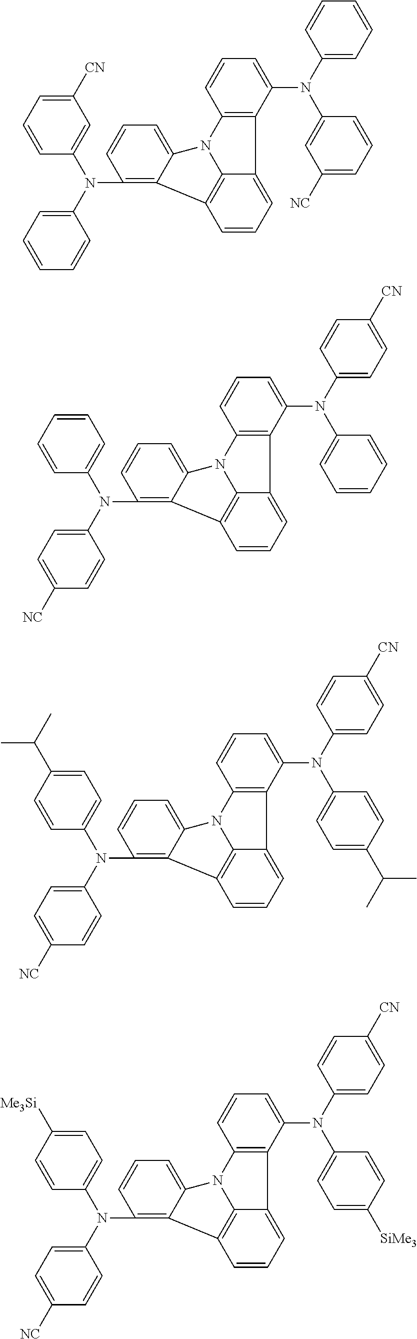 [SMILES: N#Cc1cccc(N(c2ccccc2)c2cccc3c2c2cccc4c5c(N(c6ccccc6)c6cccc(C#N)c6)cccc5n3c24)c1 ; N#Cc1ccc(N(c2ccccc2)c2cccc3c2c2cccc4c5c(N(c6ccccc6)c6ccc(C#N)cc6)cccc5n3c24)cc1 ; CC(C)c1ccc(N(c2ccc(C#N)cc2)c2cccc3c2c2cccc4c5c(N(c6ccc(C#N)cc6)c6ccc(C(C)C)cc6)cccc5n3c24)cc1 ; C[Si](C)(C)c1ccc(N(c2ccc(C#N)cc2)c2cccc3c2c2cccc4c5c(N(c6ccc(C#N)cc6)c6ccc([Si](C)(C)C)cc6)cccc5n3c24)cc1]

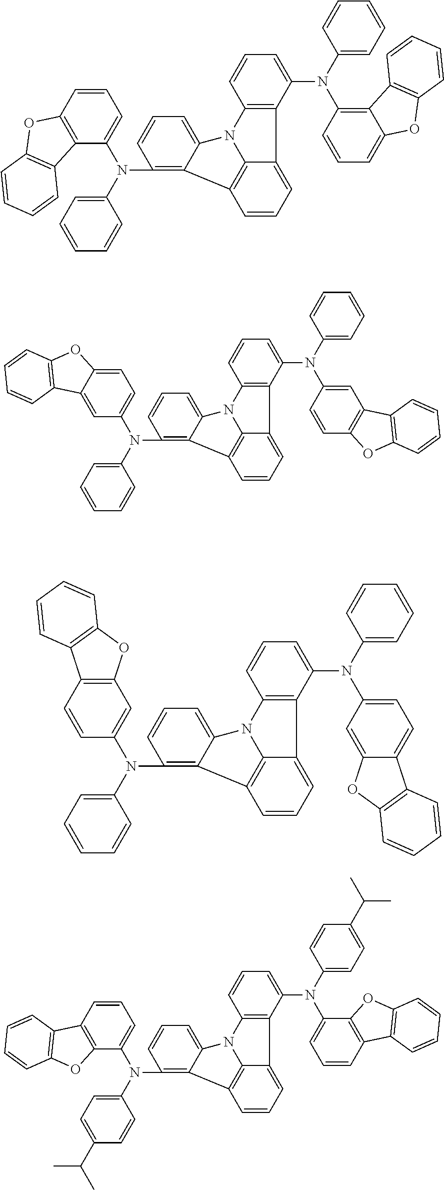 [SMILES: c1ccc(N(c2cccc3oc4ccccc4c23)c2cccc3c2c2cccc4c5c(N(c6ccccc6)c6cccc7oc8ccccc8c67)cccc5n3c24)cc1 ; c1ccc(N(c2ccc3oc4ccccc4c3c2)c2cccc3c2c2cccc4c5c(N(c6ccccc6)c6ccc7oc8ccccc8c7c6)cccc5n3c24)cc1 ; c1ccc(N(c2ccc3c(c2)oc2ccccc23)c2cccc3c2c2cccc4c5c(N(c6ccccc6)c6ccc7c(c6)oc6ccccc67)cccc5n3c24)cc1 ; CC(C)c1ccc(N(c2cccc3c2oc2ccccc23)c2cccc3c2c2cccc4c5c(N(c6ccc(C(C)C)cc6)c6cccc7c6oc6ccccc67)cccc5n3c24)cc1]

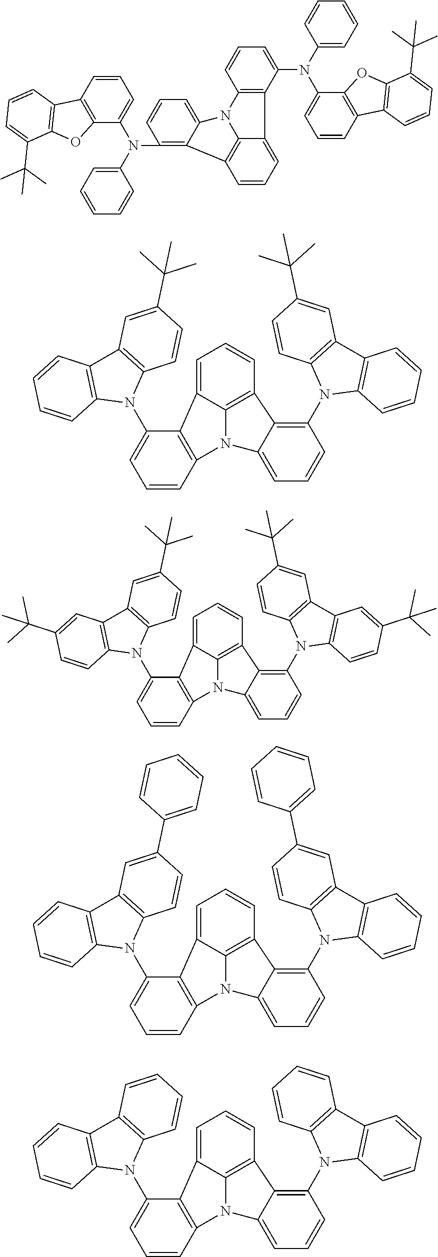

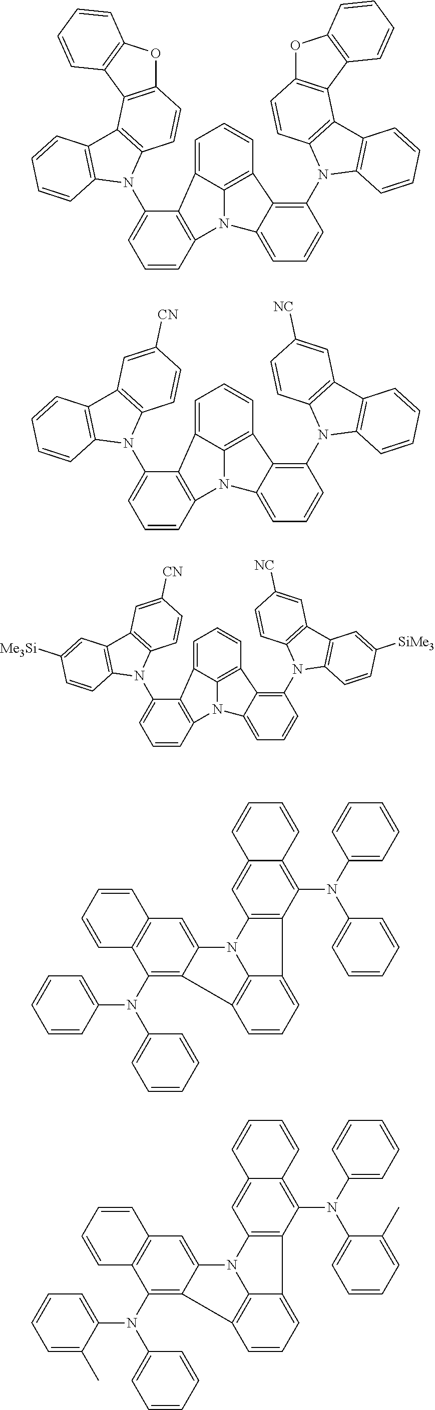

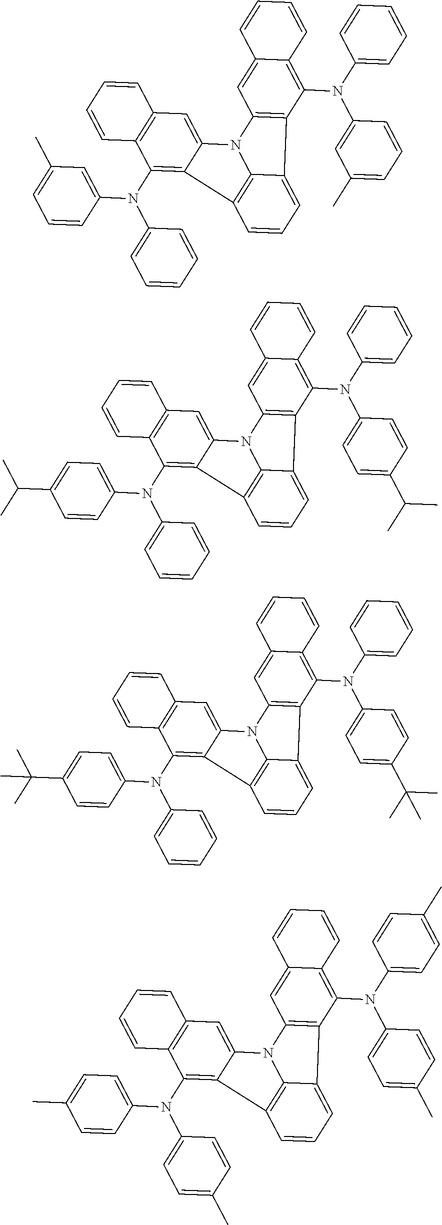

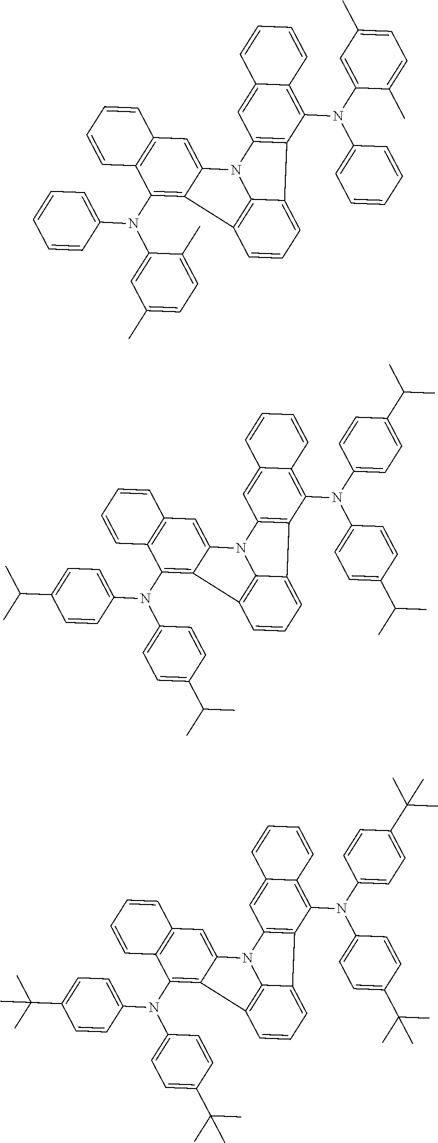

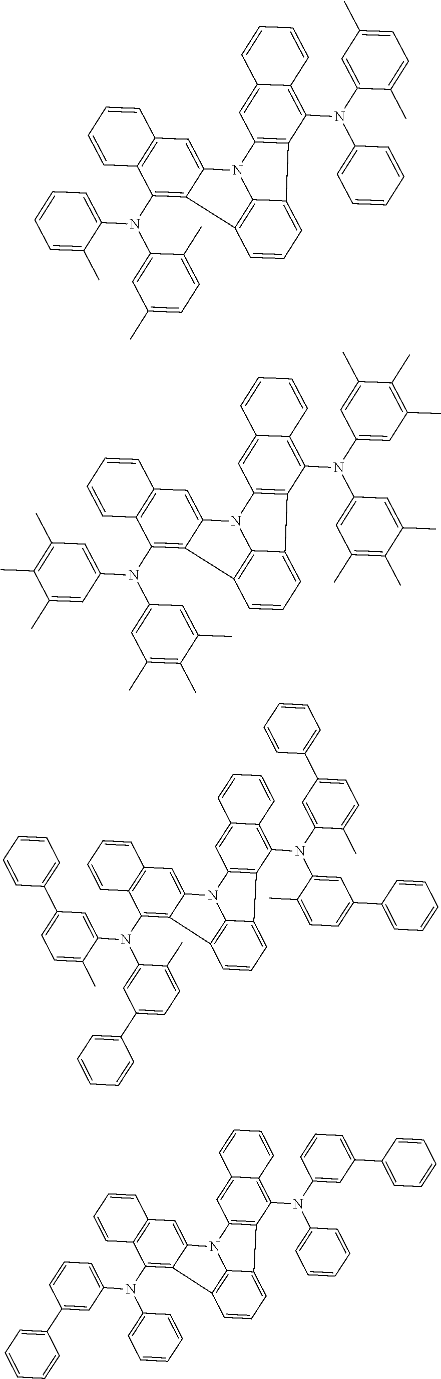

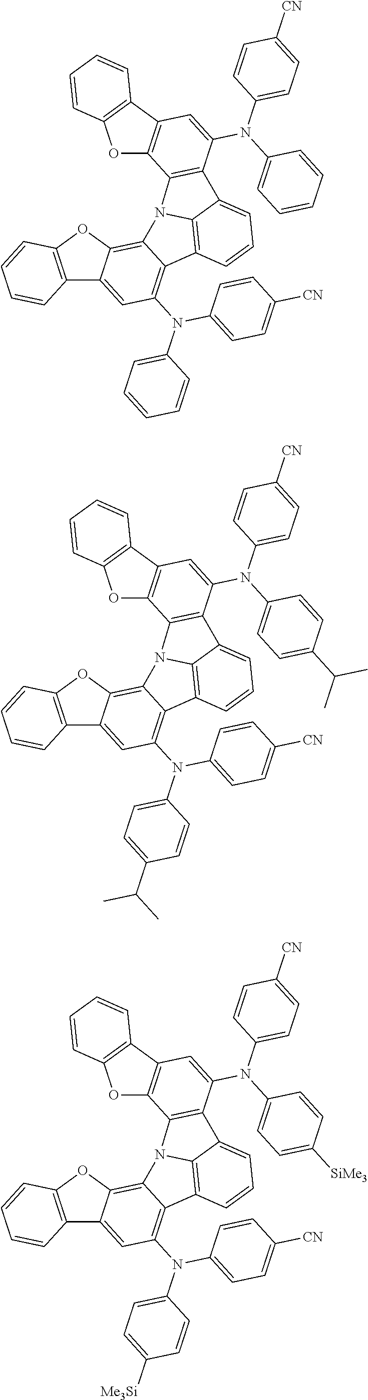 [SMILES: N#Cc1ccc(N(c2ccccc2)c2cc3c4ccccc4oc3c3c2c2cccc4c5c(N(c6ccccc6)c6ccc(C#N)cc6)cc6c7ccccc7oc6c5n3c24)cc1 ; CC(C)c1ccc(N(c2ccc(C#N)cc2)c2cc3c4ccccc4oc3c3c2c2cccc4c5c(N(c6ccc(C#N)cc6)c6ccc(C(C)C)cc6)cc6c7ccccc7oc6c5n3c24)cc1 ; C[Si](C)(C)c1ccc(N(c2ccc(C#N)cc2)c2cc3c4ccccc4oc3c3c2c2cccc4c5c(N(c6ccc(C#N)cc6)c6ccc([Si](C)(C)C)cc6)cc6c7ccccc7oc6c5n3c24)cc1]

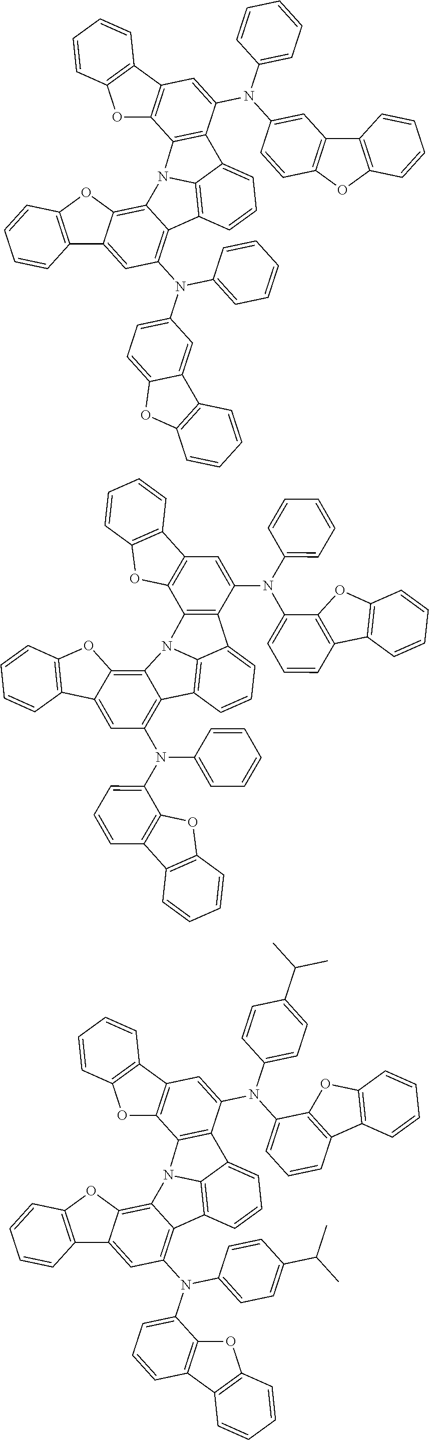

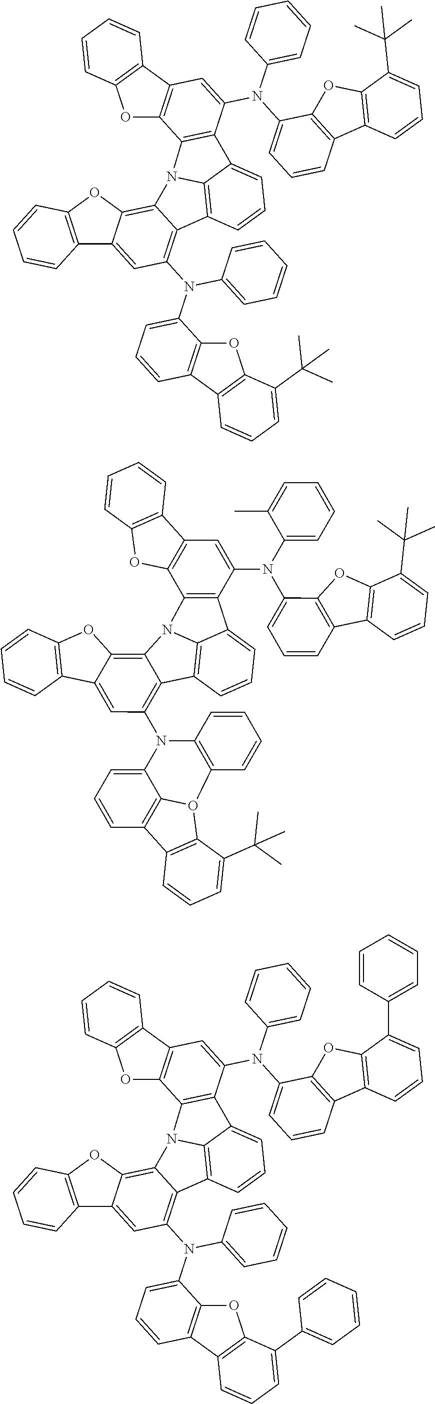

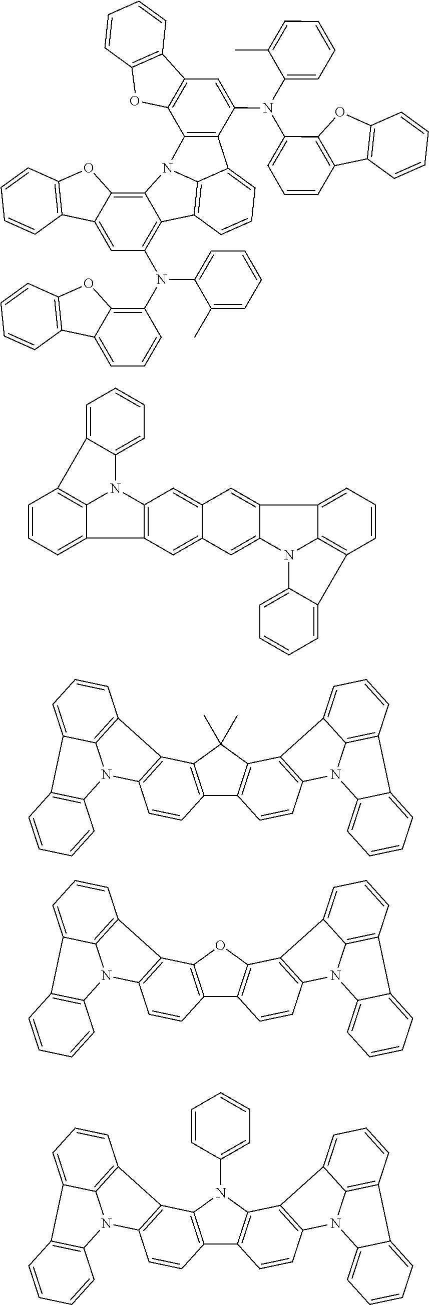

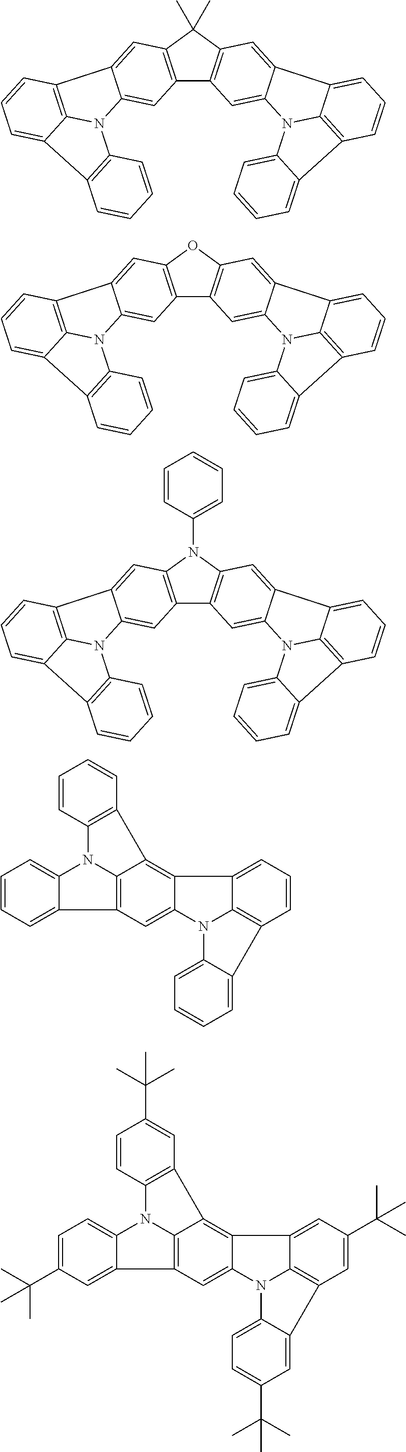

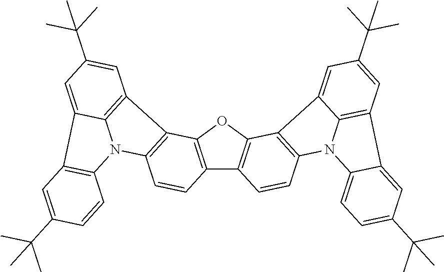 [SMILES: CC(C)(C)c1ccc2c(c1)c1cc(C(C)(C)C)cc3c4c5oc6c(ccc7c6c6cc(C(C)(C)C)cc8c9cc(C(C)(C)C)ccc9n7c86)c5ccc4n2c13]

### Compound Represented by Formula (5)

The compound represented by the formula (5) will be described below.

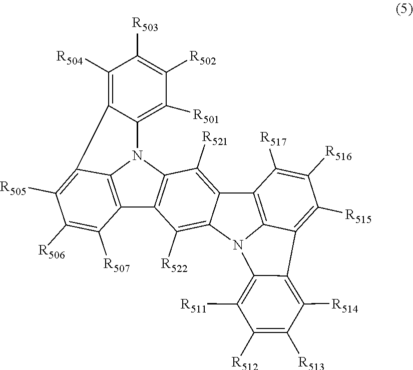

In the formula (5):
at least one combination of adjacent two or more of R₅₀₁ to R₅₀₇ and R₅₁₁ to R₅₁₇ are mutually bonded to form a substituted or unsubstituted monocyclic ring, mutually bonded to form a substituted or unsubstituted fused ring, or not mutually bonded; and
R₅₂₁, R₅₂₂, and R₅₀₁ to R₅₀₇ and R₅₁₁ to R₅₁₇ forming neither the substituted or unsubstituted monocyclic ring nor the substituted or unsubstituted fused ring are each independently a hydrogen atom, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a halogen atom, a cyano group, a nitro group, a substituted or unsubstituted aryl group having 6 to 50 ring carbon atoms, or a substituted or unsubstituted heterocyclic group having 5 to 50 ring atoms.

### Specific Examples of Compound Represented by Formula (5)

Specific examples of the compound represented by the formula (5) include compounds shown below.

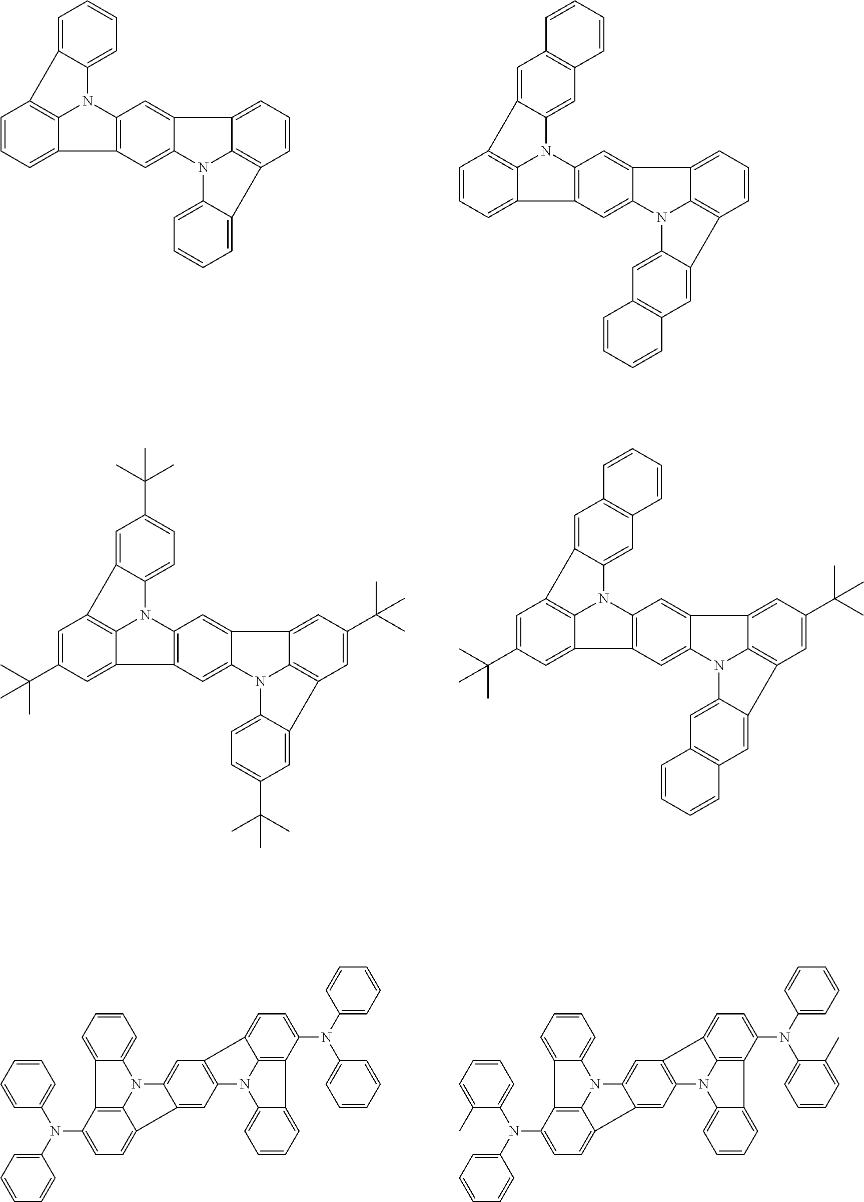 [SMILES: c1ccc2c(c1)c1cccc3c4cc5c(cc4n2c13)c1cccc2c3ccccc3n5c21 ; c1ccc2cc3c(cc2c1)c1cccc2c4cc5c(cc4n3c12)c1cccc2c3cc4ccccc4cc3n5c21 ; CC(C)(C)c1ccc2c(c1)c1cc(C(C)(C)C)cc3c4cc5c(cc4n2c13)c1cc(C(C)(C)C)cc2c3cc(C(C)(C)C)ccc3n5c21 ; CC(C)(C)c1cc2c3cc4ccccc4cc3n3c4cc5c6cc(C(C)(C)C)cc7c8cc9ccccc9cc8n(c5cc4c(c1)c23)c76 ; c1ccc(N(c2ccccc2)c2ccc3c4cc5c(cc4n4c6ccccc6c2c34)c2ccc(N(c3ccccc3)c3ccccc3)c3c4ccccc4n5c23)cc1 ; Cc1ccccc1N(c1ccccc1)c1ccc2c3cc4c(cc3n3c5ccccc5c1c23)c1ccc(N(c2ccccc2)c2ccccc2C)c2c3ccccc3n4c12]

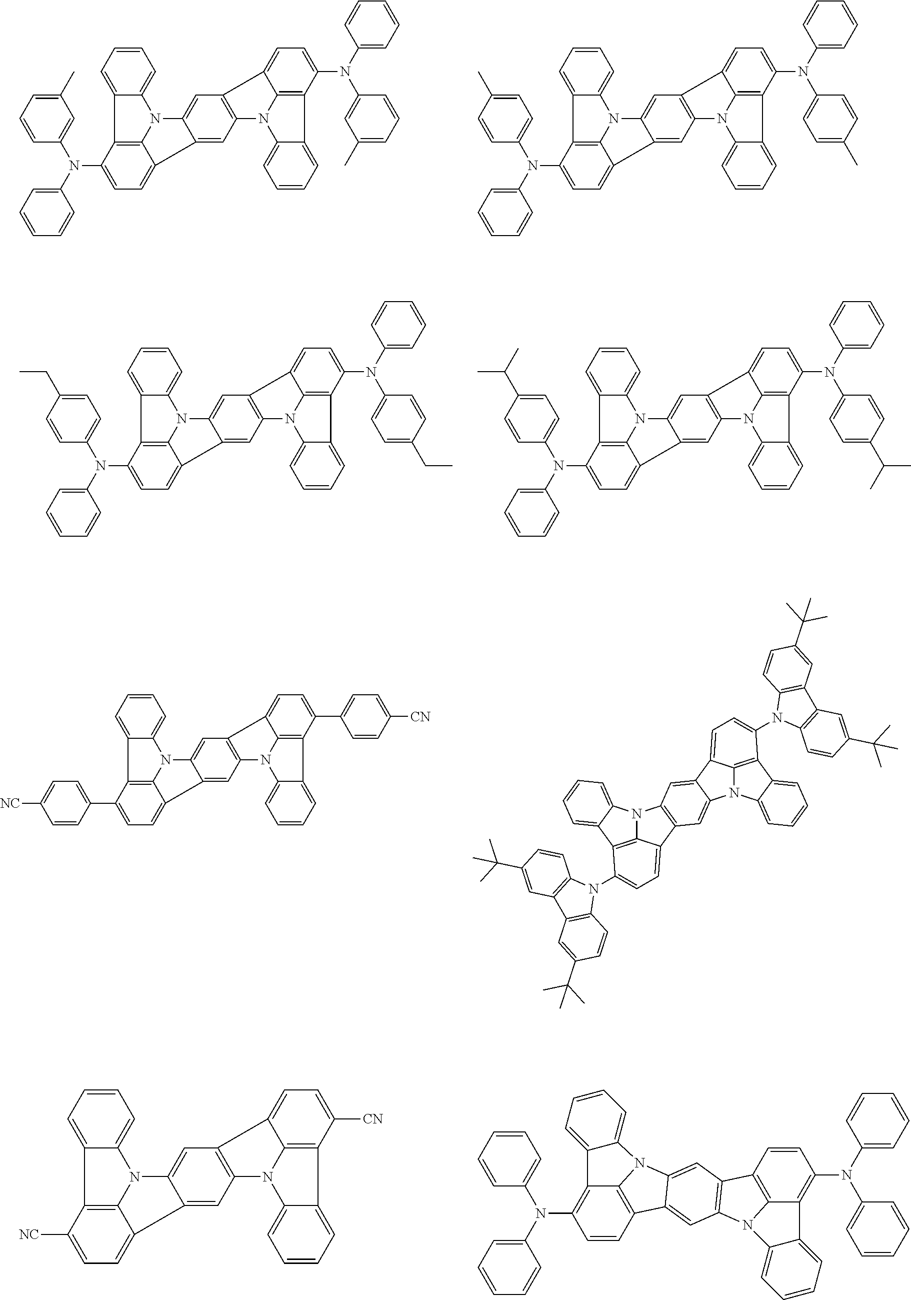

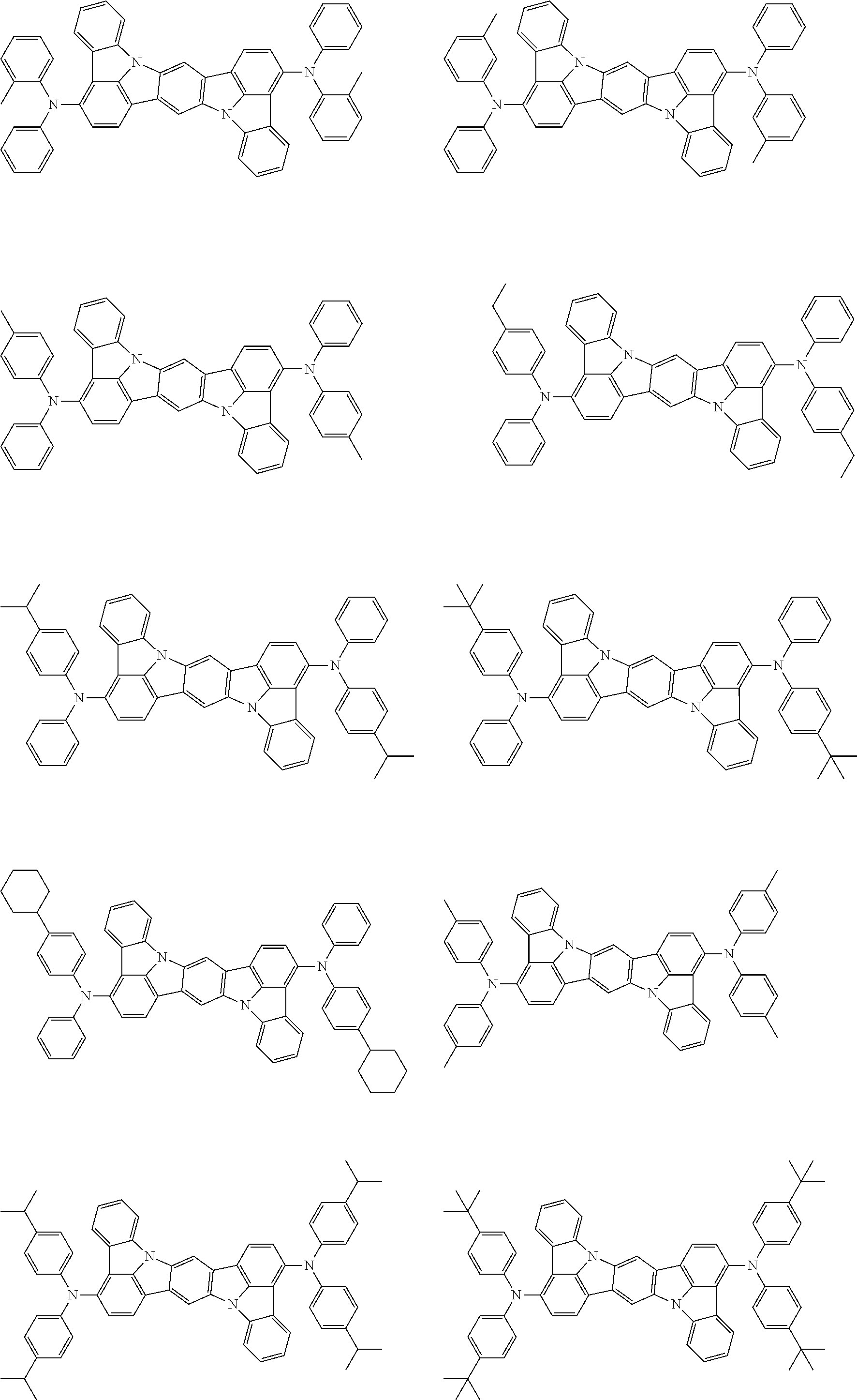

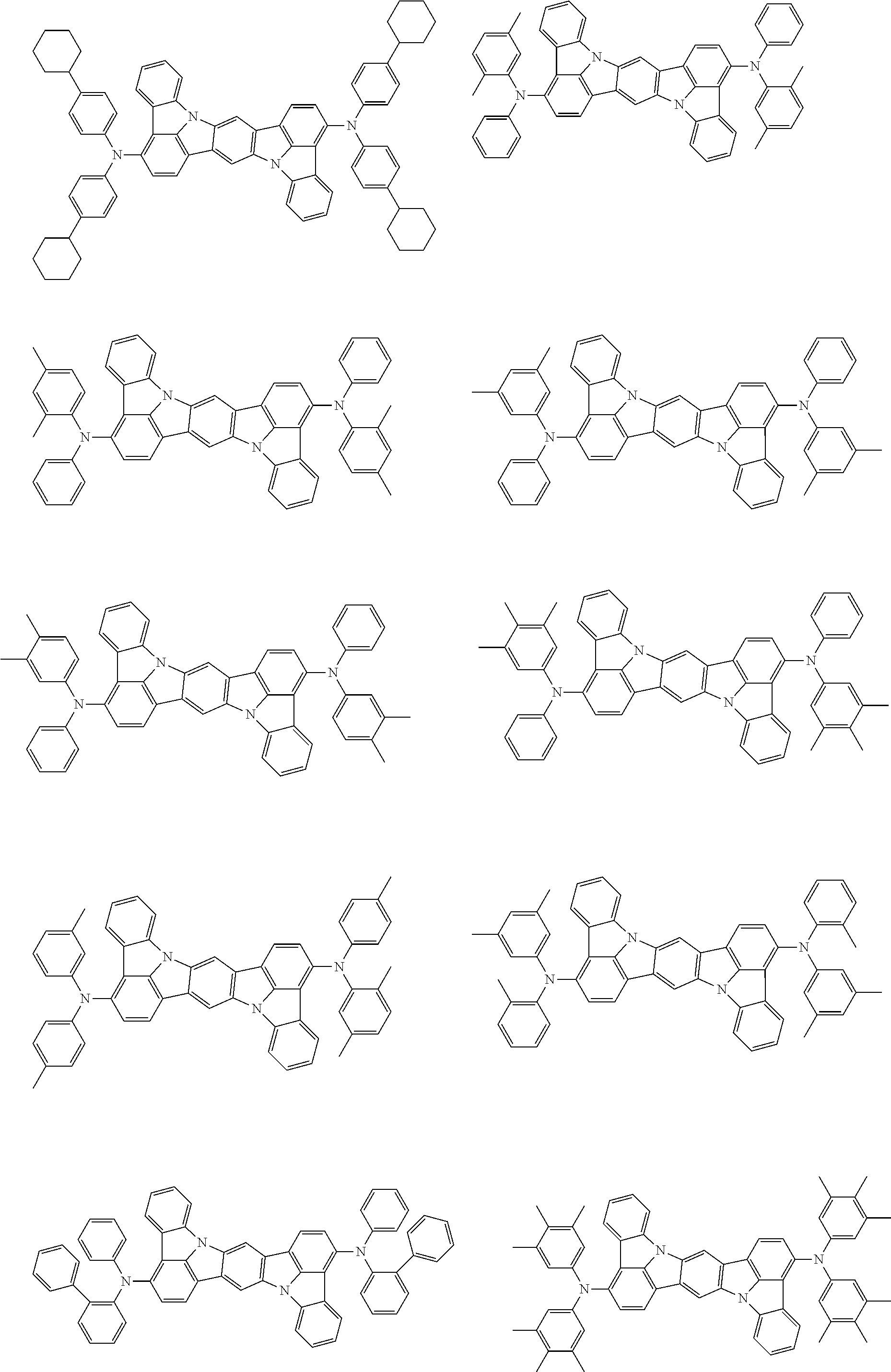

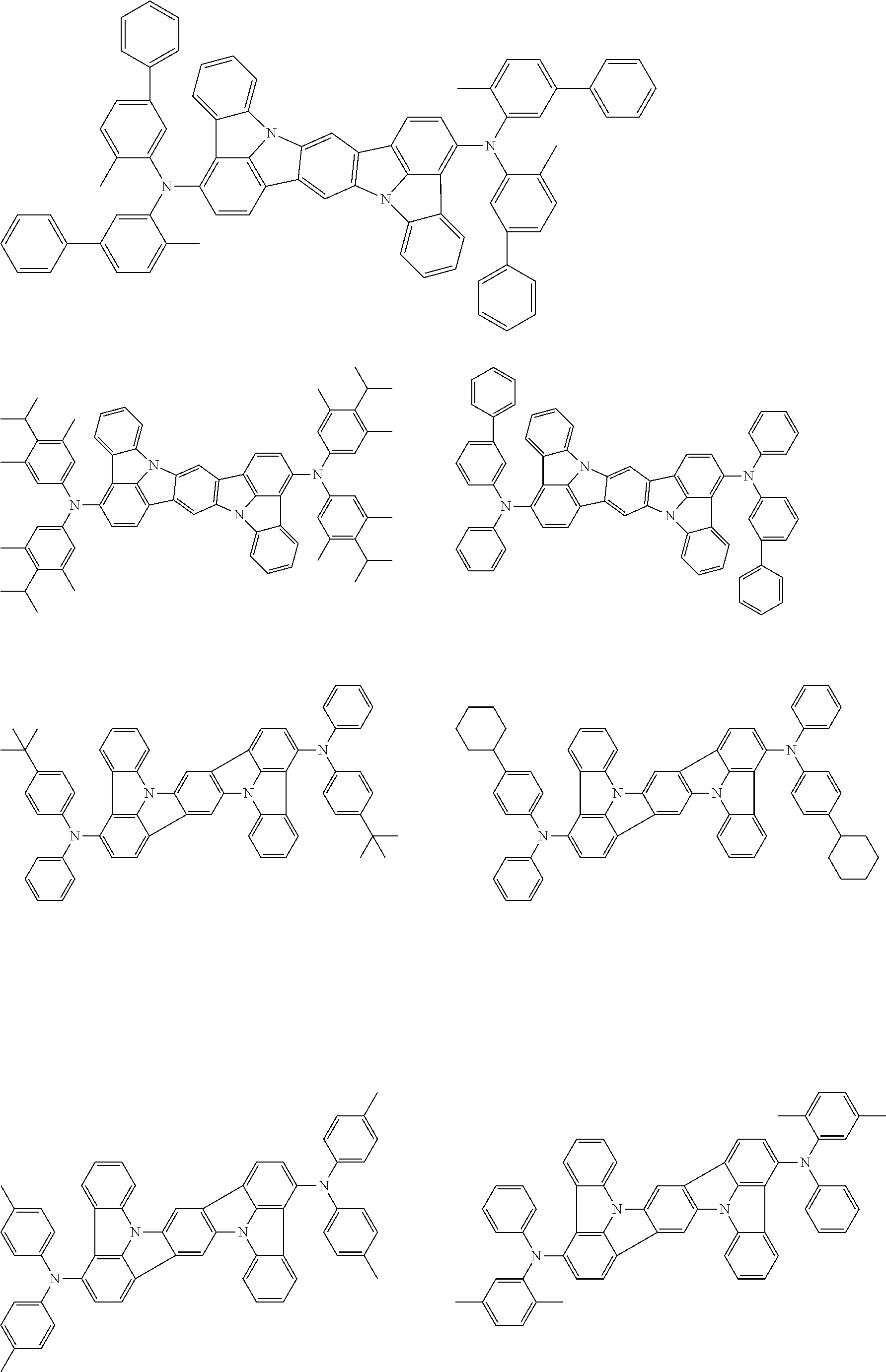

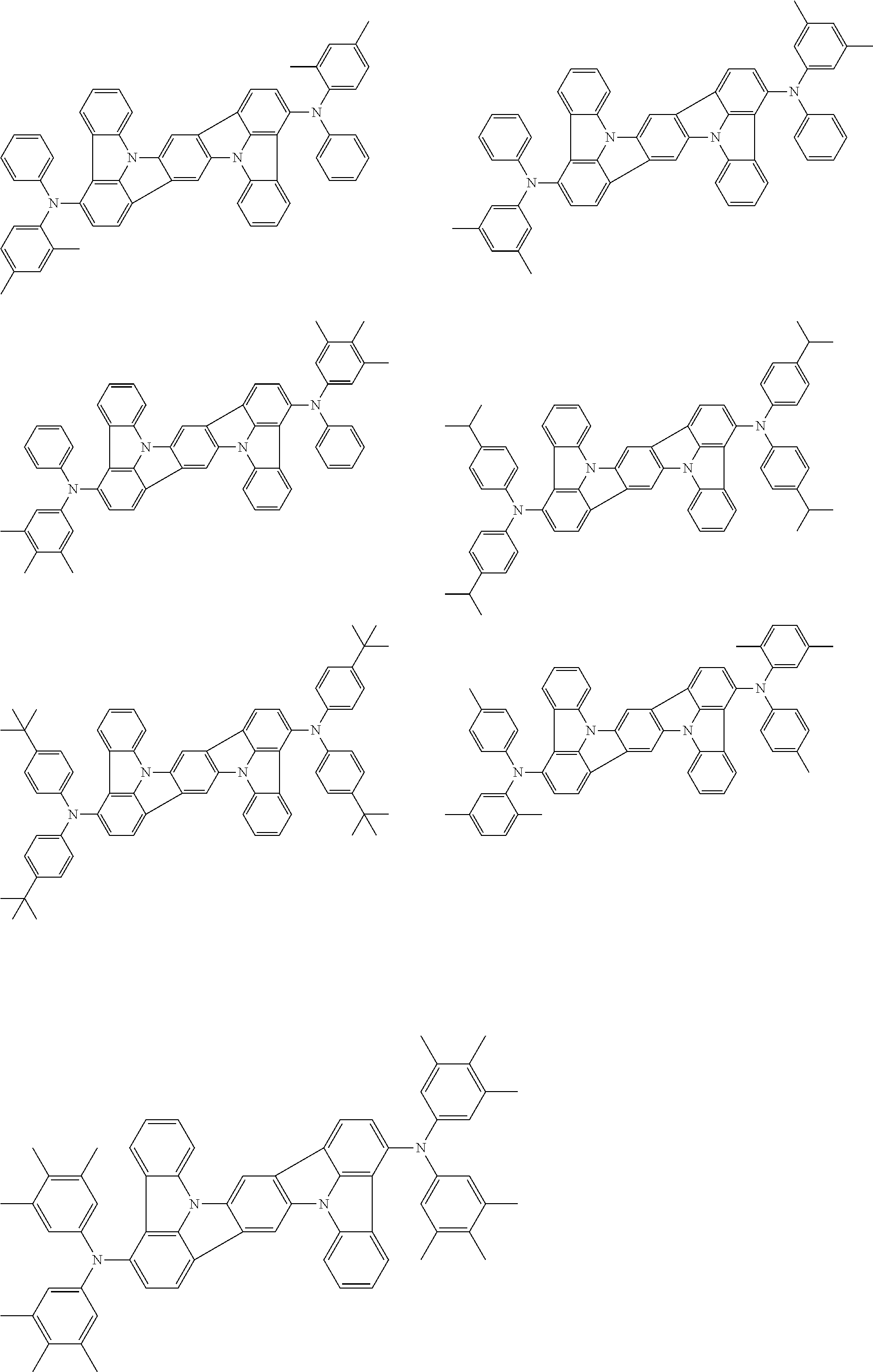

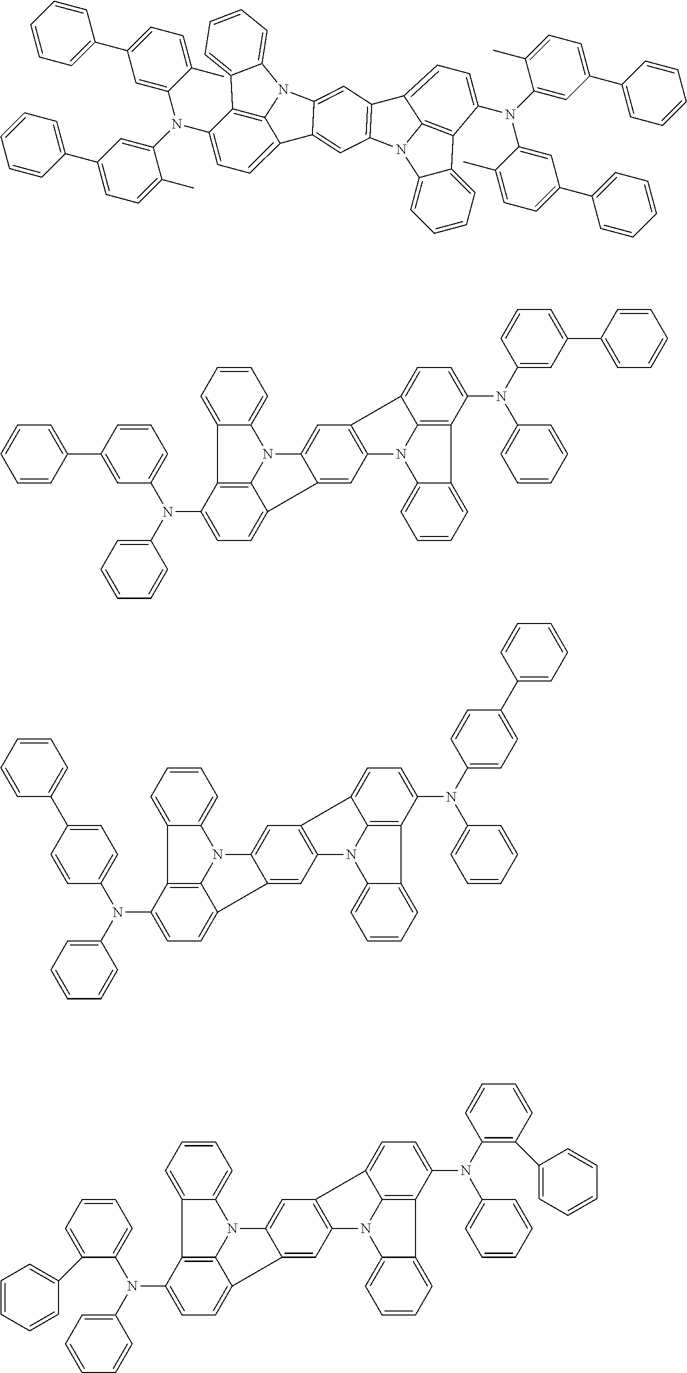 [SMILES: Cc1ccc(-c2ccccc2)cc1N(c1cc(-c2ccccc2)ccc1C)c1ccc2c3cc4c(cc3n3c5ccccc5c1c23)c1ccc(N(c2cc(-c3ccccc3)ccc2C)c2cc(-c3ccccc3)ccc2C)c2c3ccccc3n4c12 ; c1ccc(-c2cccc(N(c3ccccc3)c3ccc4c5cc6c(cc5n5c7ccccc7c3c45)c3ccc(N(c4ccccc4)c4cccc(-c5ccccc5)c4)c4c5ccccc5n6c34)c2)cc1 ; c1ccc(-c2ccc(N(c3ccccc3)c3ccc4c5cc6c(cc5n5c7ccccc7c3c45)c3ccc(N(c4ccccc4)c4ccc(-c5ccccc5)cc4)c4c5ccccc5n6c34)cc2)cc1 ; c1ccc(-c2ccccc2N(c2ccccc2)c2ccc3c4cc5c(cc4n4c6ccccc6c2c34)c2ccc(N(c3ccccc3)c3ccccc3-c3ccccc3)c3c4ccccc4n5c23)cc1]

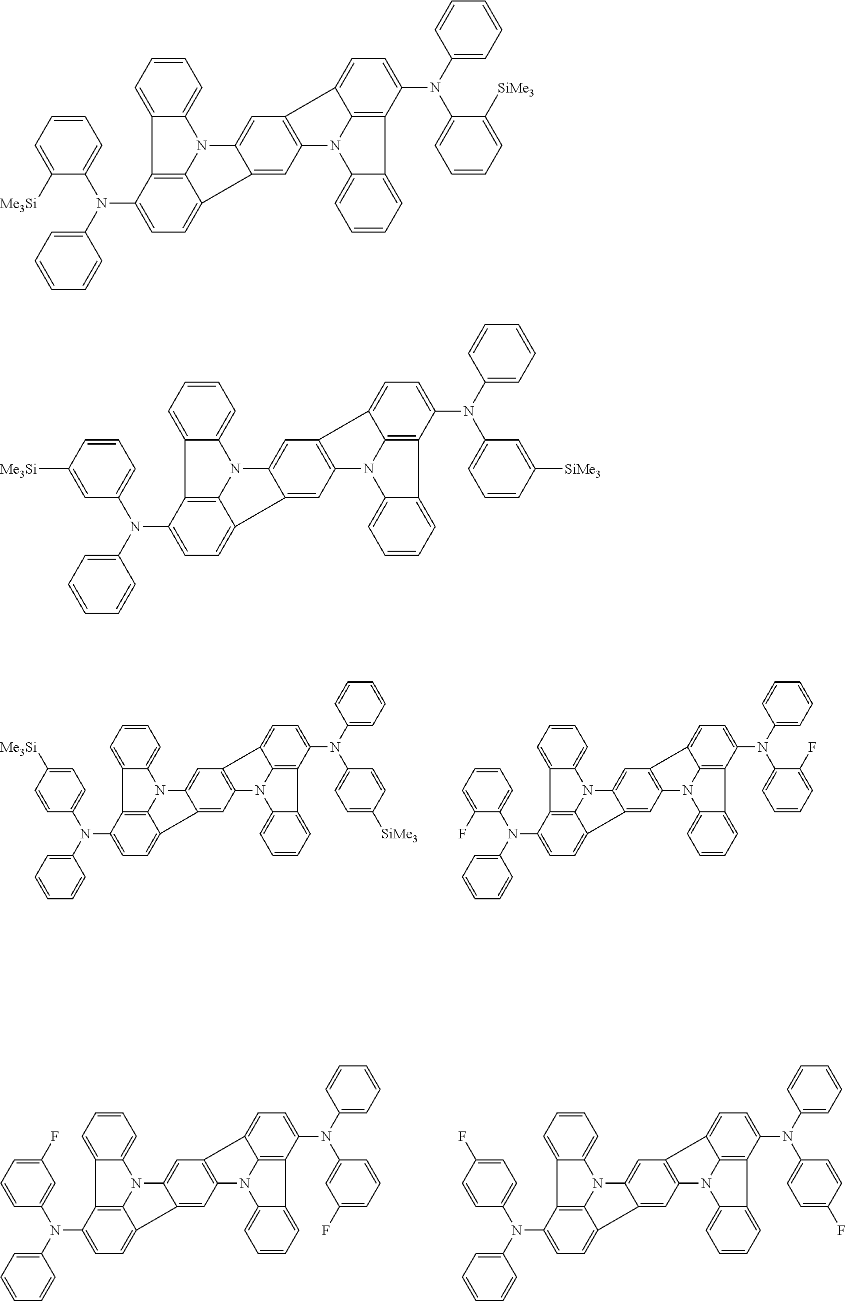

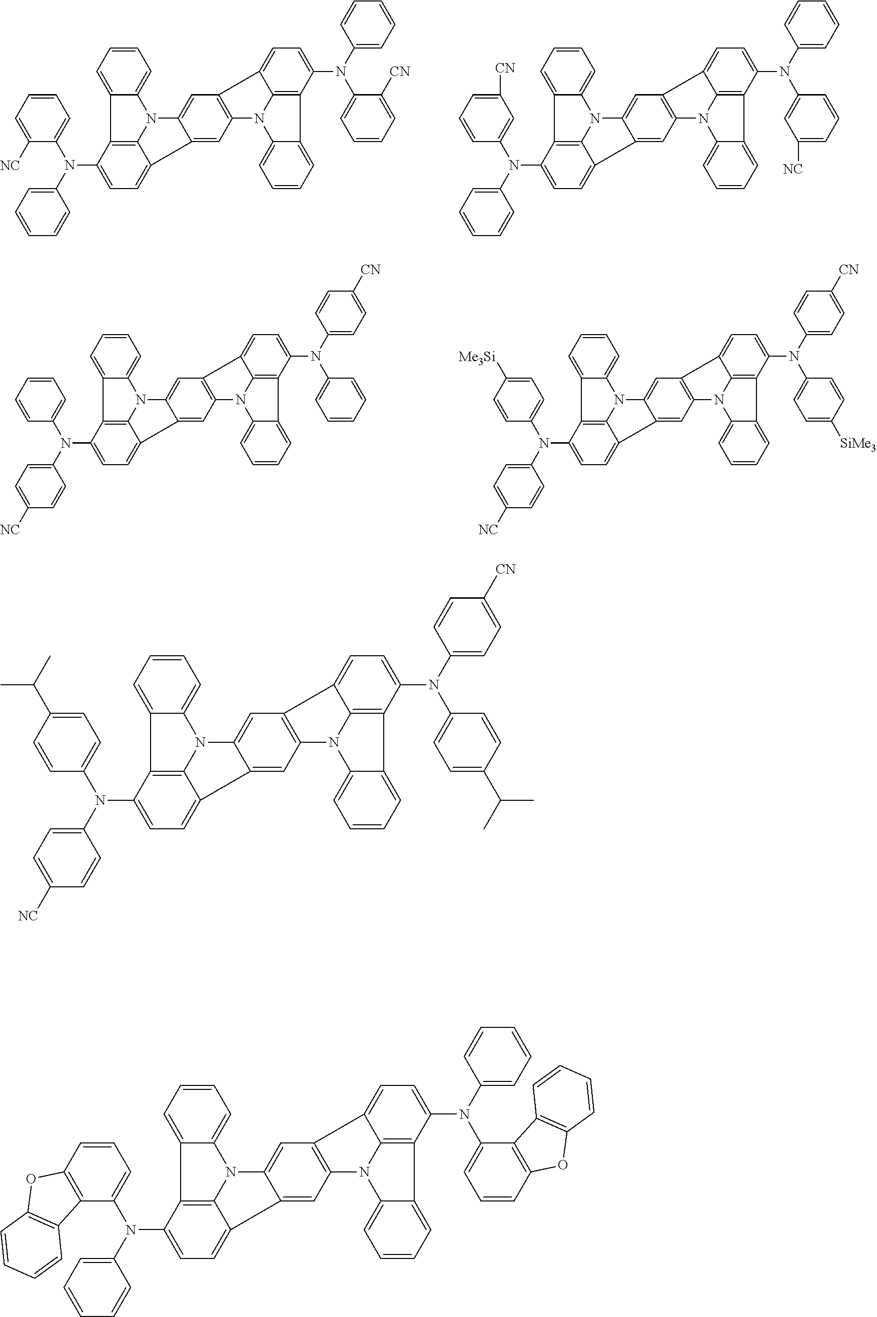

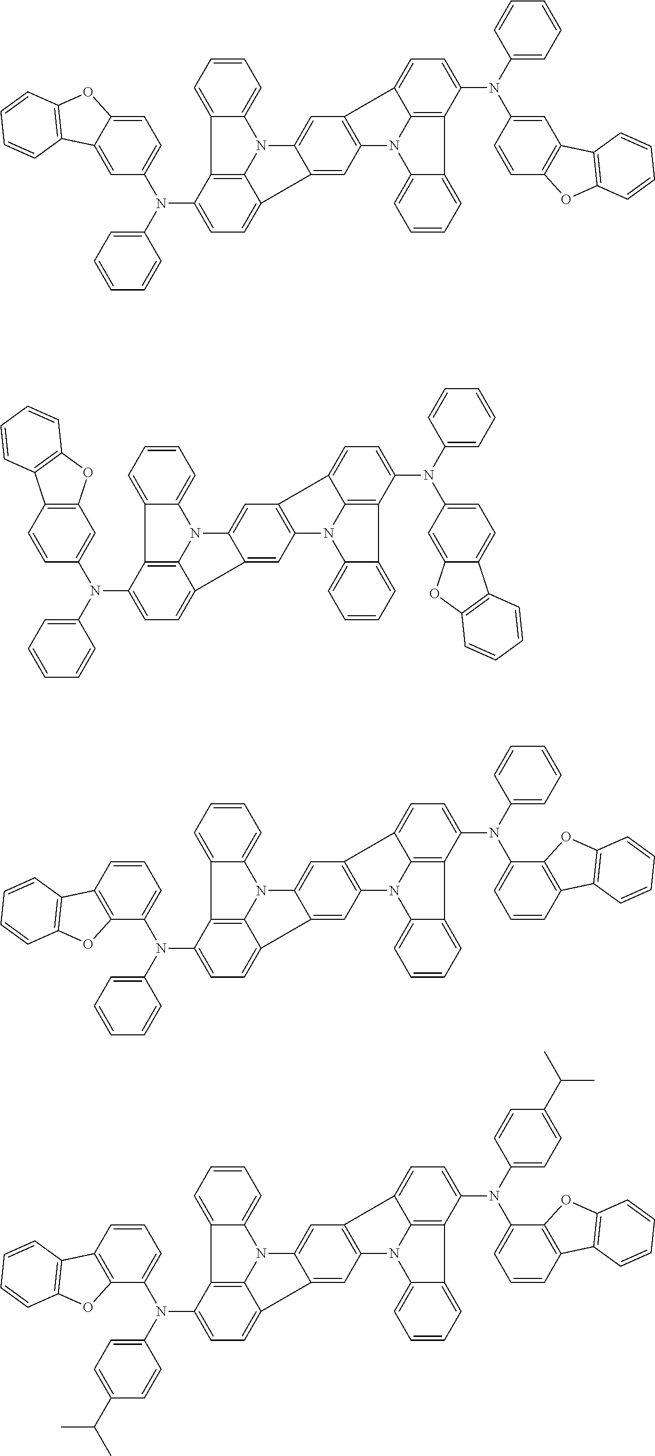 [SMILES: c1ccc(N(c2ccc3oc4ccccc4c3c2)c2ccc3c4cc5c(cc4n4c6ccccc6c2c34)c2ccc(N(c3ccccc3)c3ccc4oc6ccccc6c4c3)c3c4ccccc4n5c23)cc1 ; c1ccc(N(c2ccc3c(c2)oc2ccccc23)c2ccc3c4cc5c(cc4n4c6ccccc6c2c34)c2ccc(N(c3ccccc3)c3ccc4c(c3)oc3ccccc34)c3c4ccccc4n5c23)cc1 ; c1ccc(N(c2cccc3c2oc2ccccc23)c2ccc3c4cc5c(cc4n4c6ccccc6c2c34)c2ccc(N(c3ccccc3)c3cccc4c3oc3ccccc34)c3c4ccccc4n5c23)cc1 ; CC(C)c1ccc(N(c2cccc3c2oc2ccccc23)c2ccc3c4cc5c(cc4n4c6ccccc6c2c34)c2ccc(N(c3ccc(C(C)C)cc3)c3cccc4c3oc3ccccc34)c3c4ccccc4n5c23)cc1]

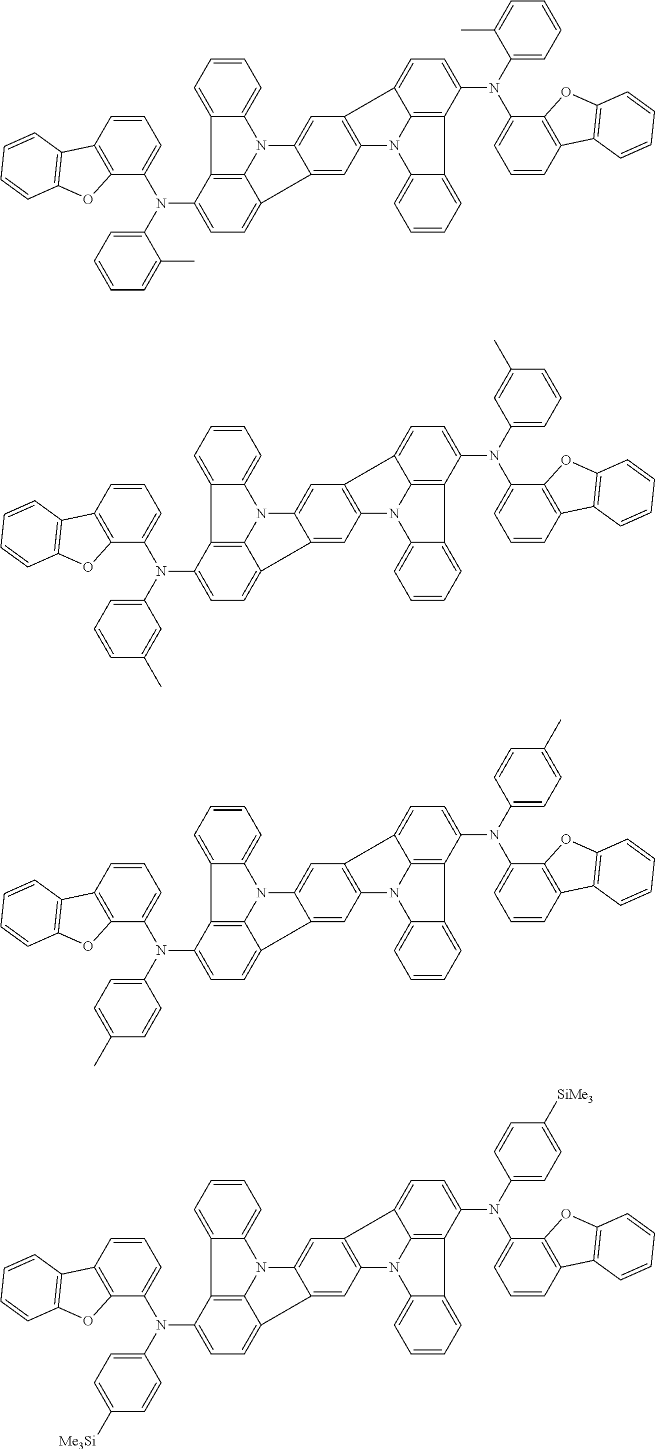

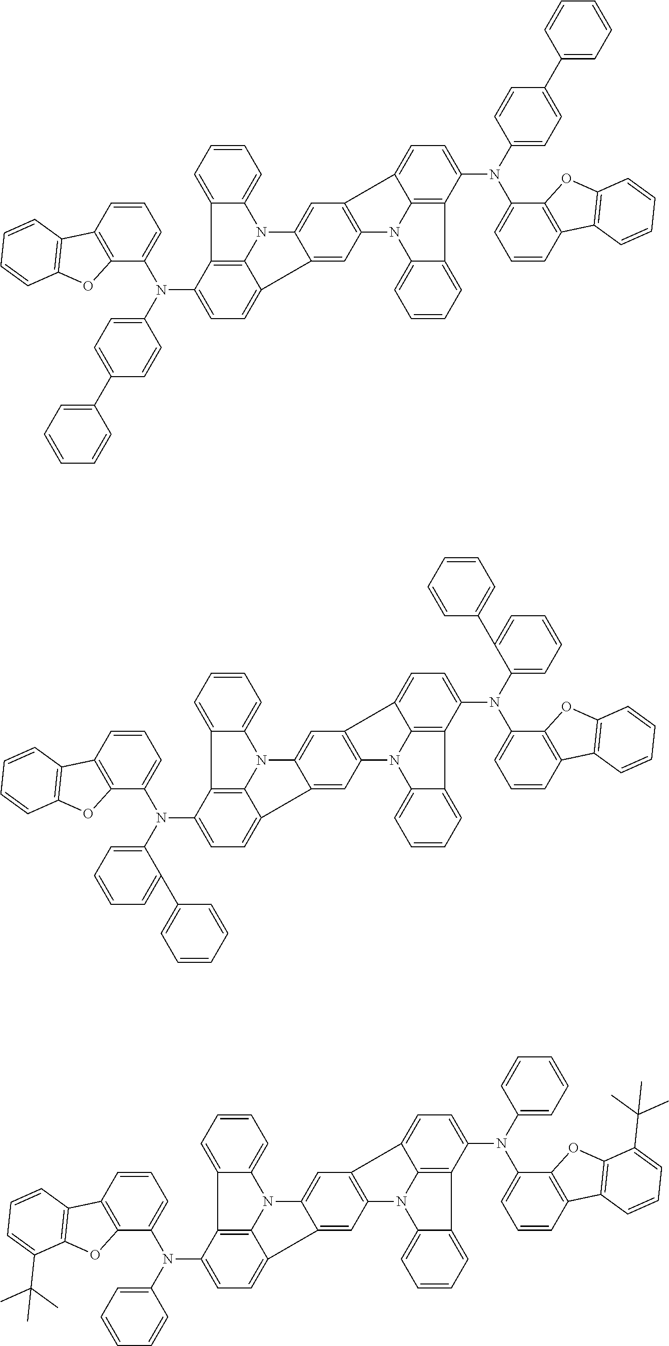 [SMILES: c1ccc(-c2ccc(N(c3cccc4c3oc3ccccc34)c3ccc4c5cc6c(cc5n5c7ccccc7c3c45)c3ccc(N(c4ccc(-c5ccccc5)cc4)c4cccc5c4oc4ccccc45)c4c5ccccc5n6c34)cc2)cc1 ; c1ccc(-c2ccccc2N(c2cccc3c2oc2ccccc23)c2ccc3c4cc5c(cc4n4c6ccccc6c2c34)c2ccc(N(c3ccccc3-c3ccccc3)c3cccc4c3oc3ccccc34)c3c4ccccc4n5c23)cc1 ; CC(C)(C)c1cccc2c1oc1c(N(c3ccccc3)c3ccc4c5cc6c(cc5n5c7ccccc7c3c45)c3ccc(N(c4ccccc4)c4cccc5c4oc4c(C(C)(C)C)cccc45)c4c5ccccc5n6c34)cccc12]

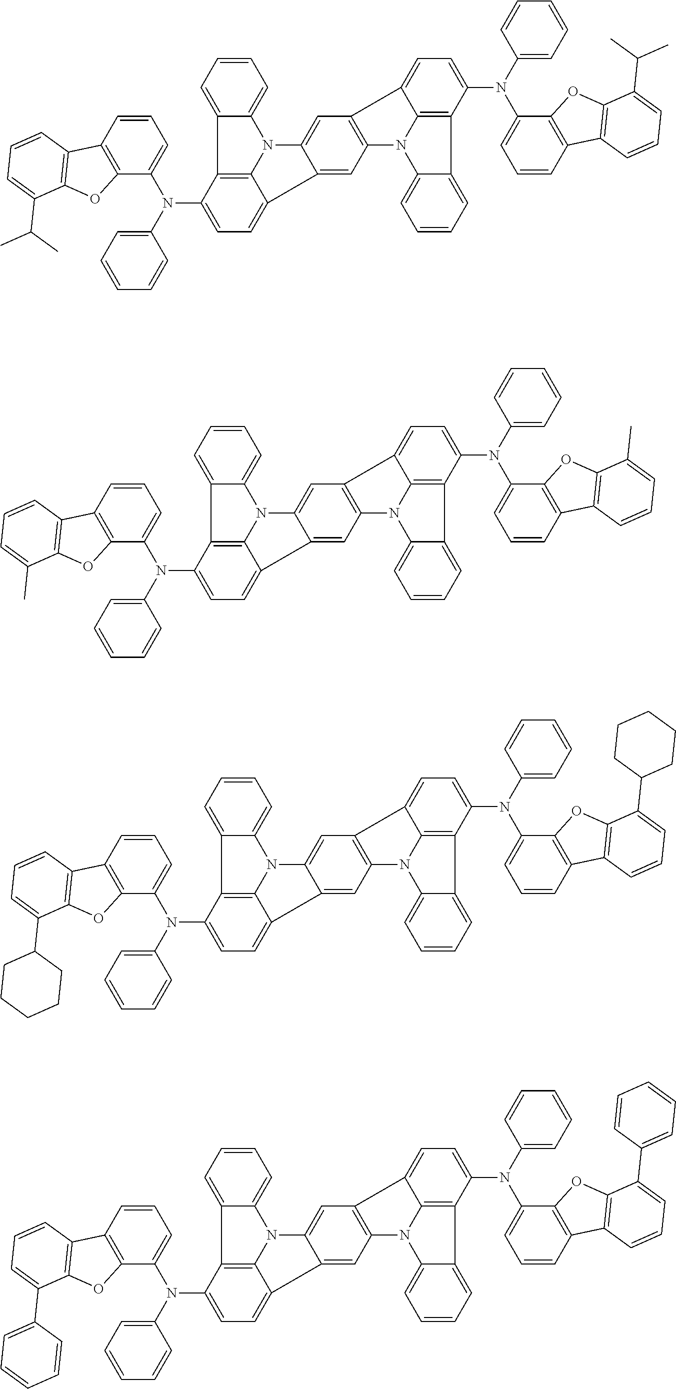

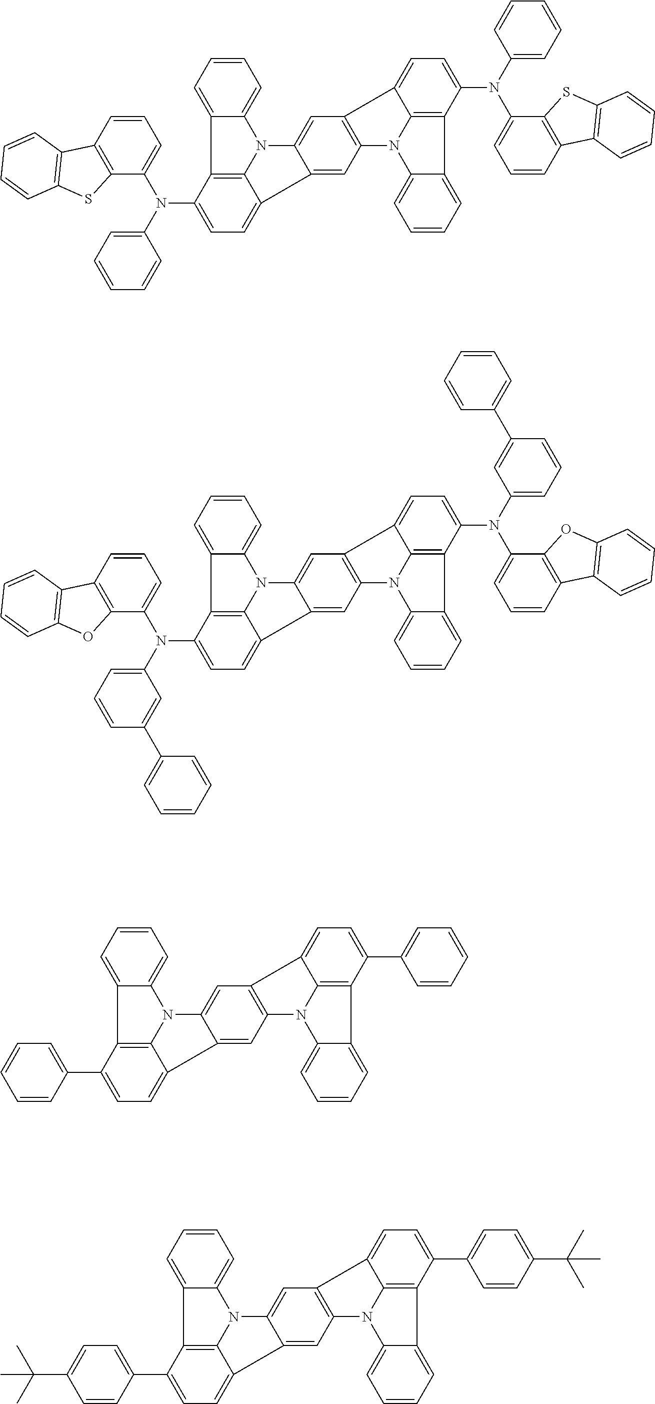 [SMILES: c1ccc(N(c2cccc3c2sc2ccccc23)c2ccc3c4cc5c(cc4n4c6ccccc6c2c34)c2ccc(N(c3ccccc3)c3cccc4c3sc3ccccc34)c3c4ccccc4n5c23)cc1 ; c1ccc(-c2cccc(N(c3cccc4c3oc3ccccc34)c3ccc4c5cc6c(cc5n5c7ccccc7c3c45)c3ccc(N(c4cccc(-c5ccccc5)c4)c4cccc5c4oc4ccccc45)c4c5ccccc5n6c34)c2)cc1 ; c1ccc(-c2ccc3c4cc5c(cc4n4c6ccccc6c2c34)c2ccc(-c3ccccc3)c3c4ccccc4n5c23)cc1 ; CC(C)(C)c1ccc(-c2ccc3c4cc5c(cc4n4c6ccccc6c2c34)c2ccc(-c3ccc(C(C)(C)C)cc3)c3c4ccccc4n5c23)cc1]

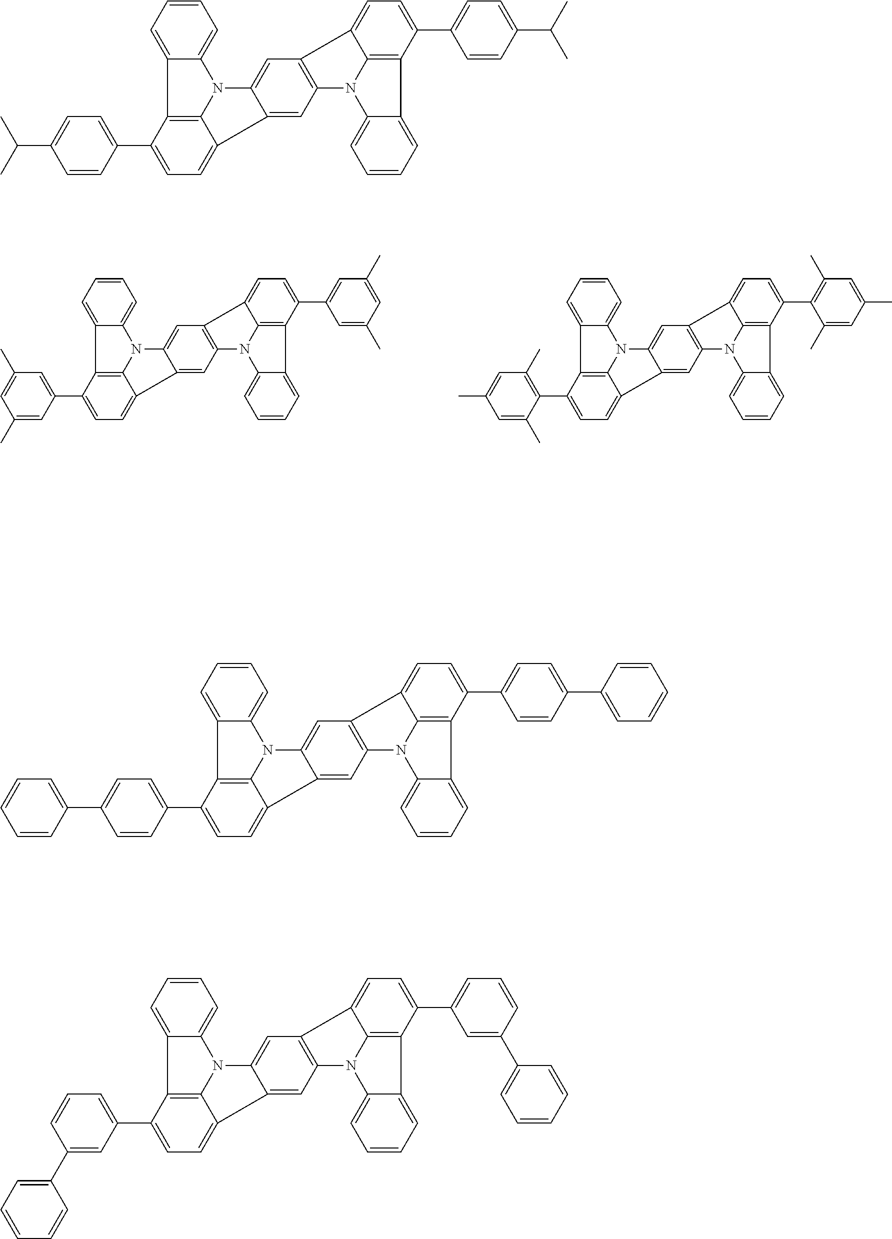

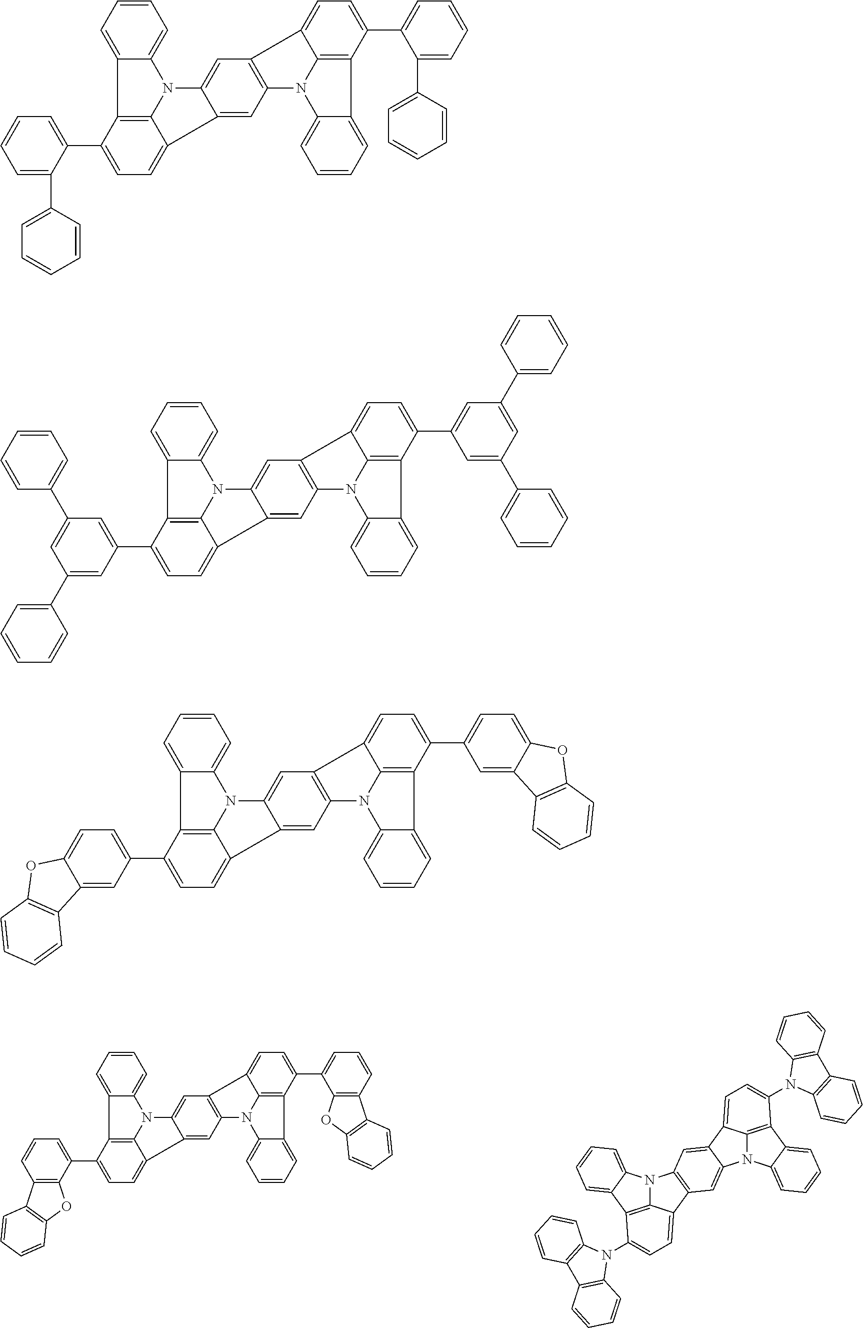

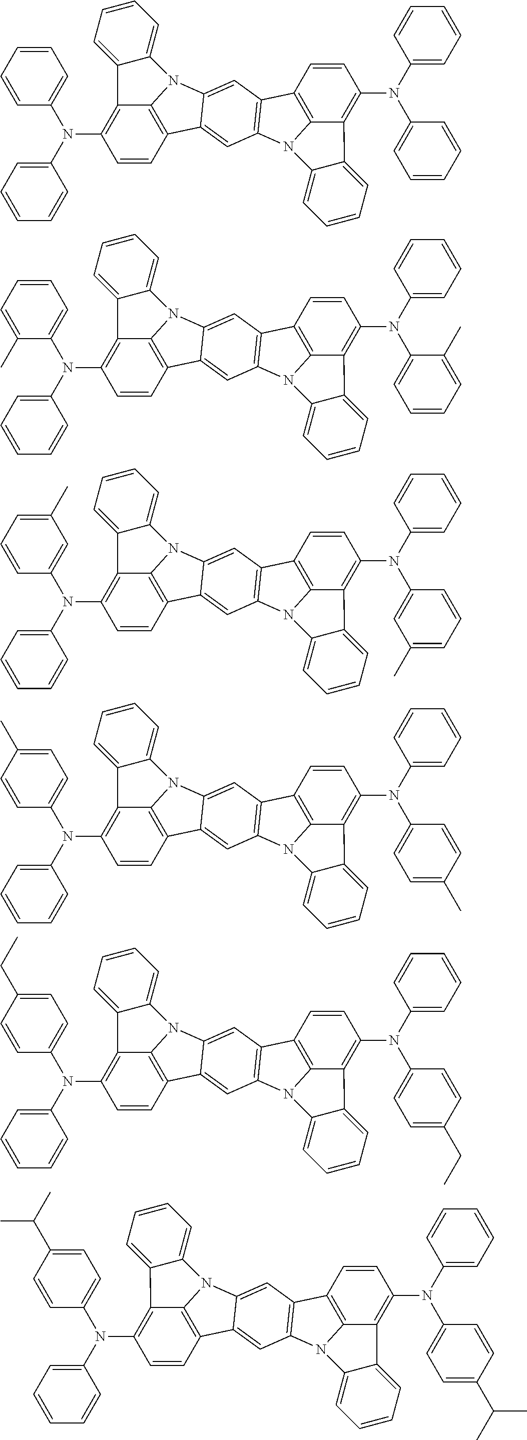

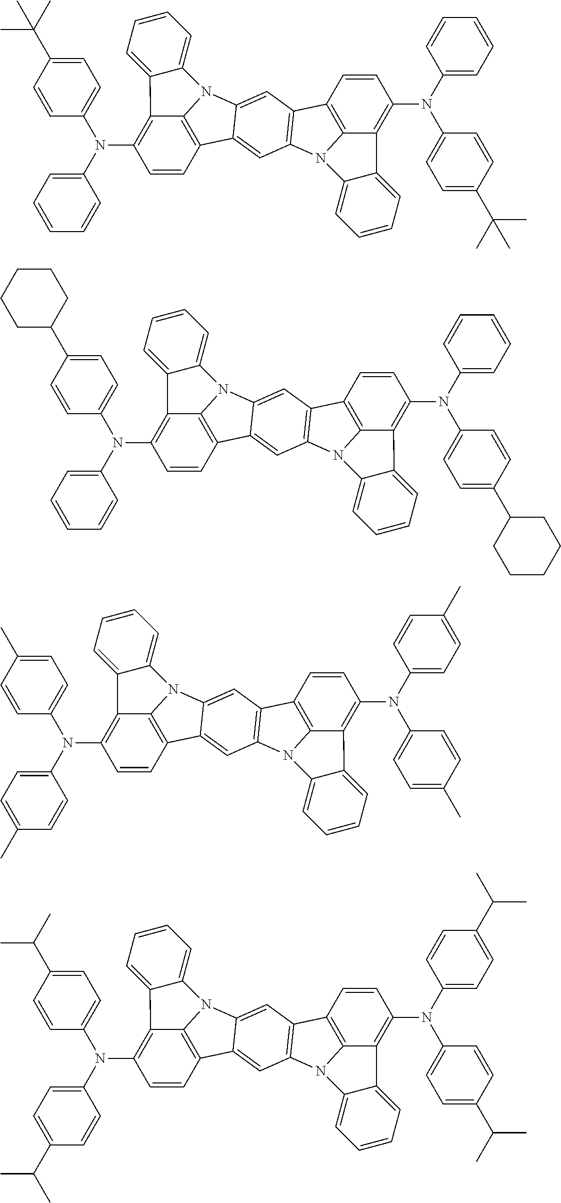

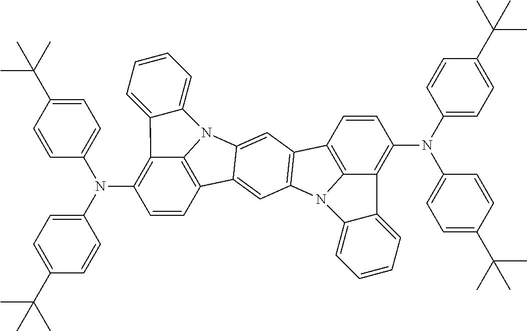 [SMILES: CC(C)(C)c1ccc(N(c2ccc(C(C)(C)C)cc2)c2ccc3c4cc5c(cc4n4c6ccccc6c2c34)c2ccc(N(c3ccc(C(C)(C)C)cc3)c3ccc(C(C)(C)C)cc3)c3c4ccccc4n5c23)cc1]

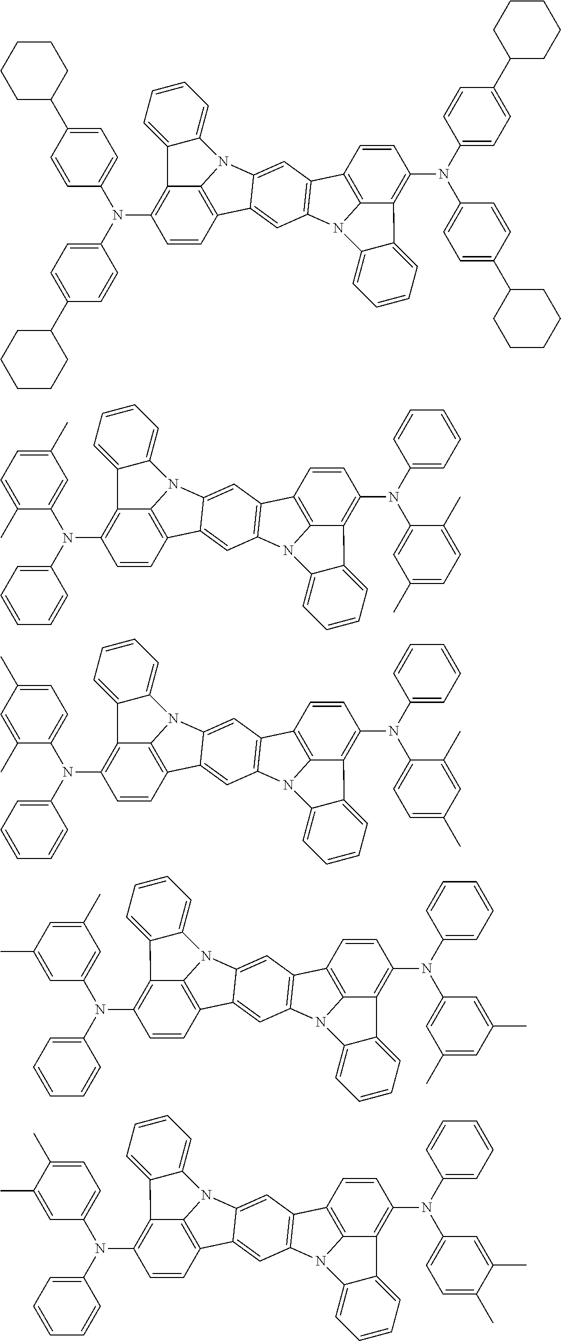

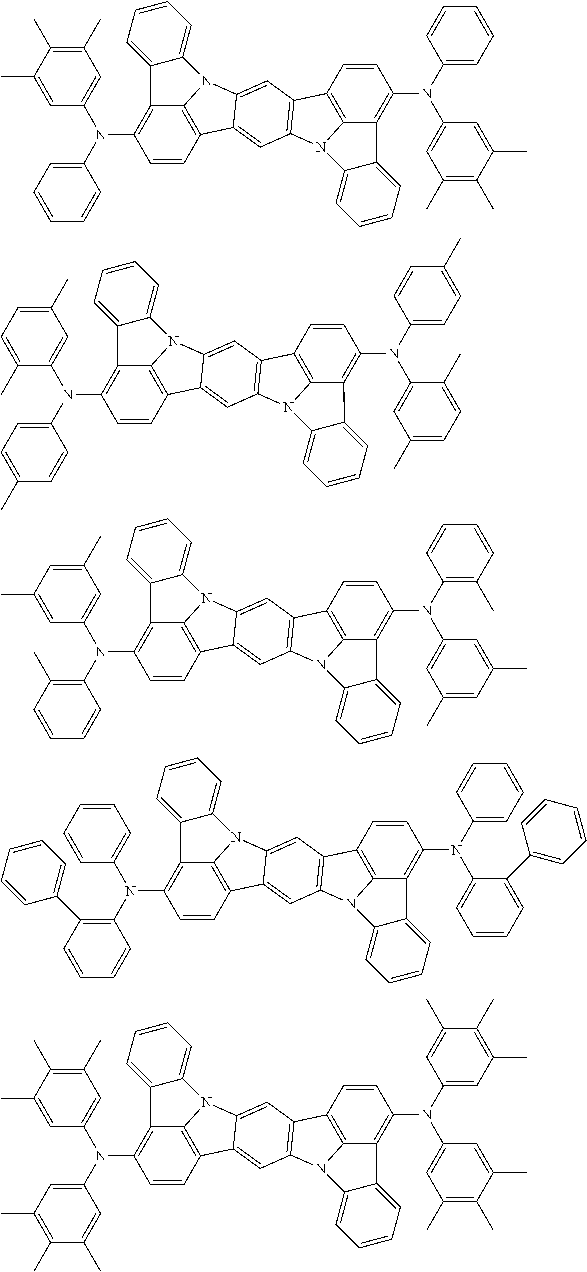

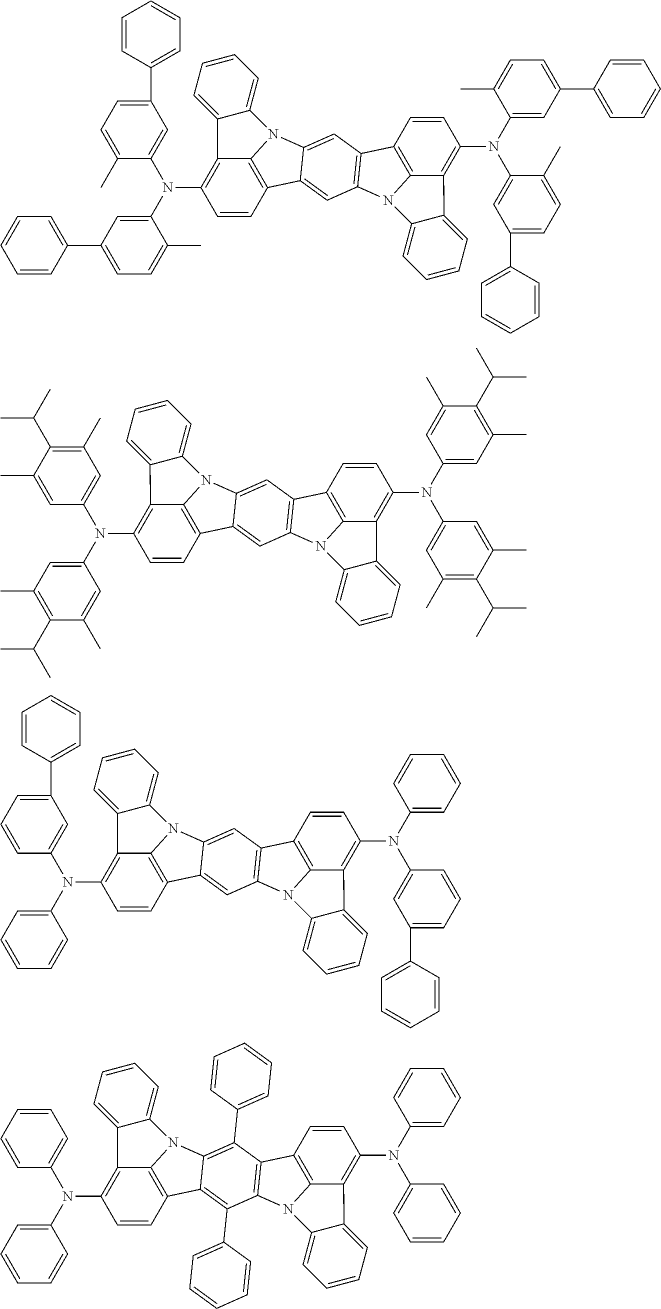

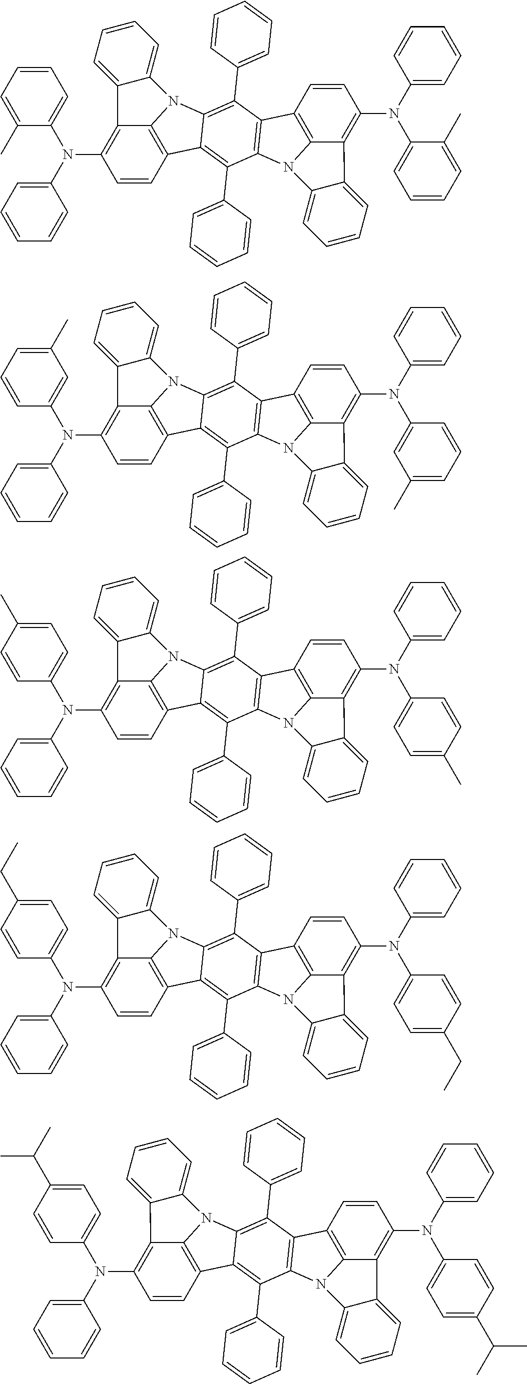

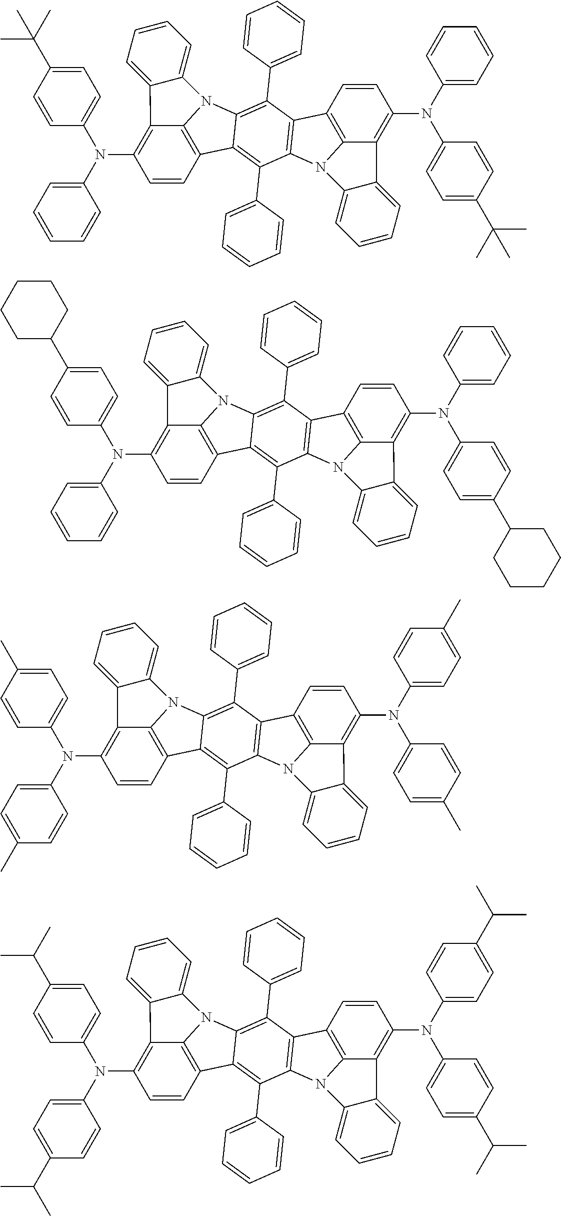

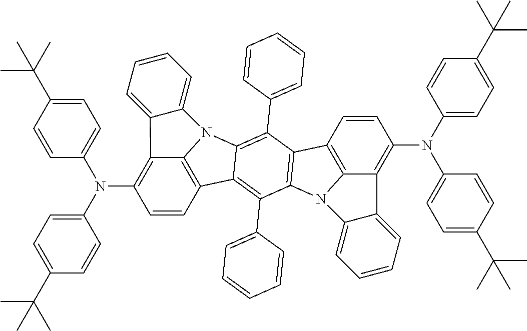 [SMILES: CC(C)(C)c1ccc(N(c2ccc(C(C)(C)C)cc2)c2ccc3c4c(-c5ccccc5)c5c(c(-c6ccccc6)c4n4c6ccccc6c2c34)c2ccc(N(c3ccc(C(C)(C)C)cc3)c3ccc(C(C)(C)C)cc3)c3c4ccccc4n5c23)cc1]

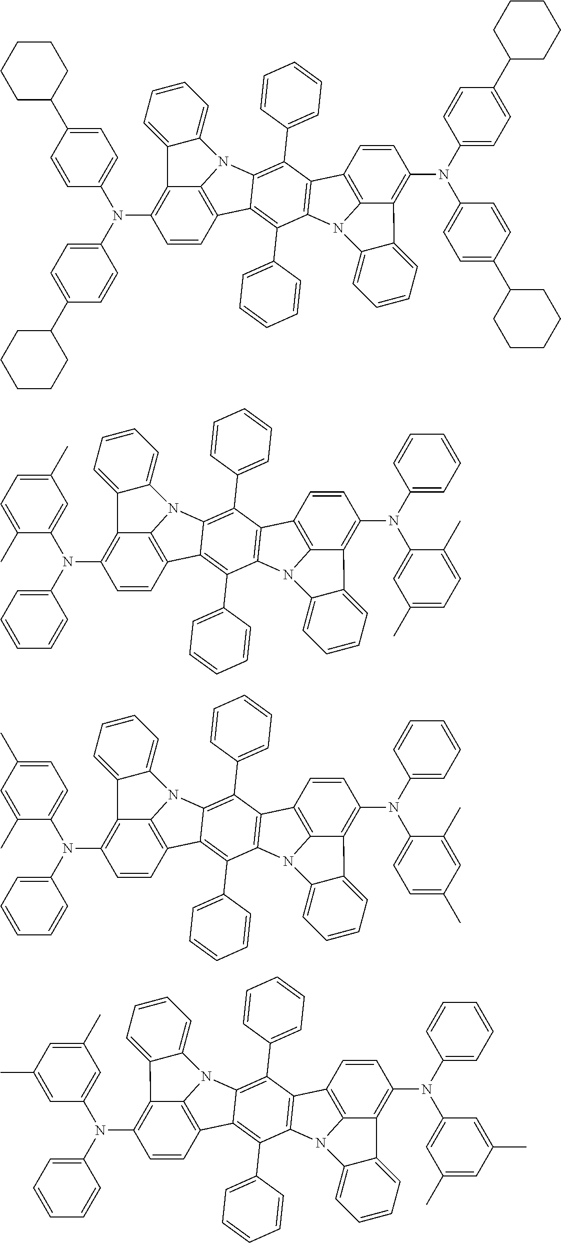

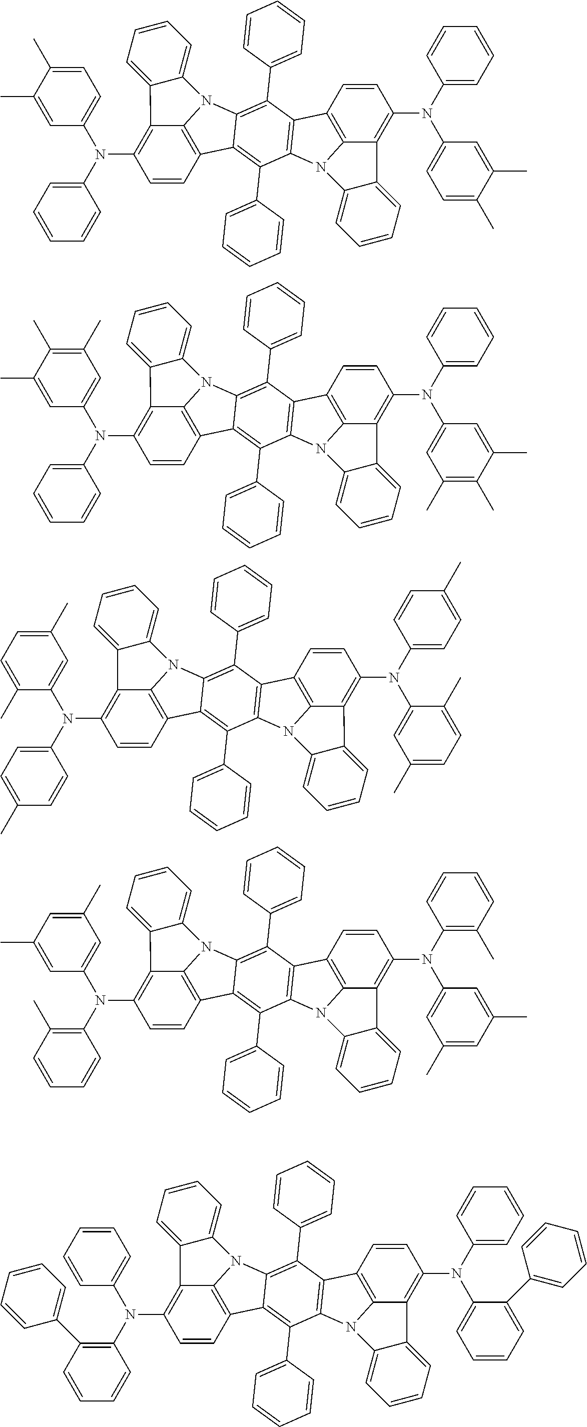

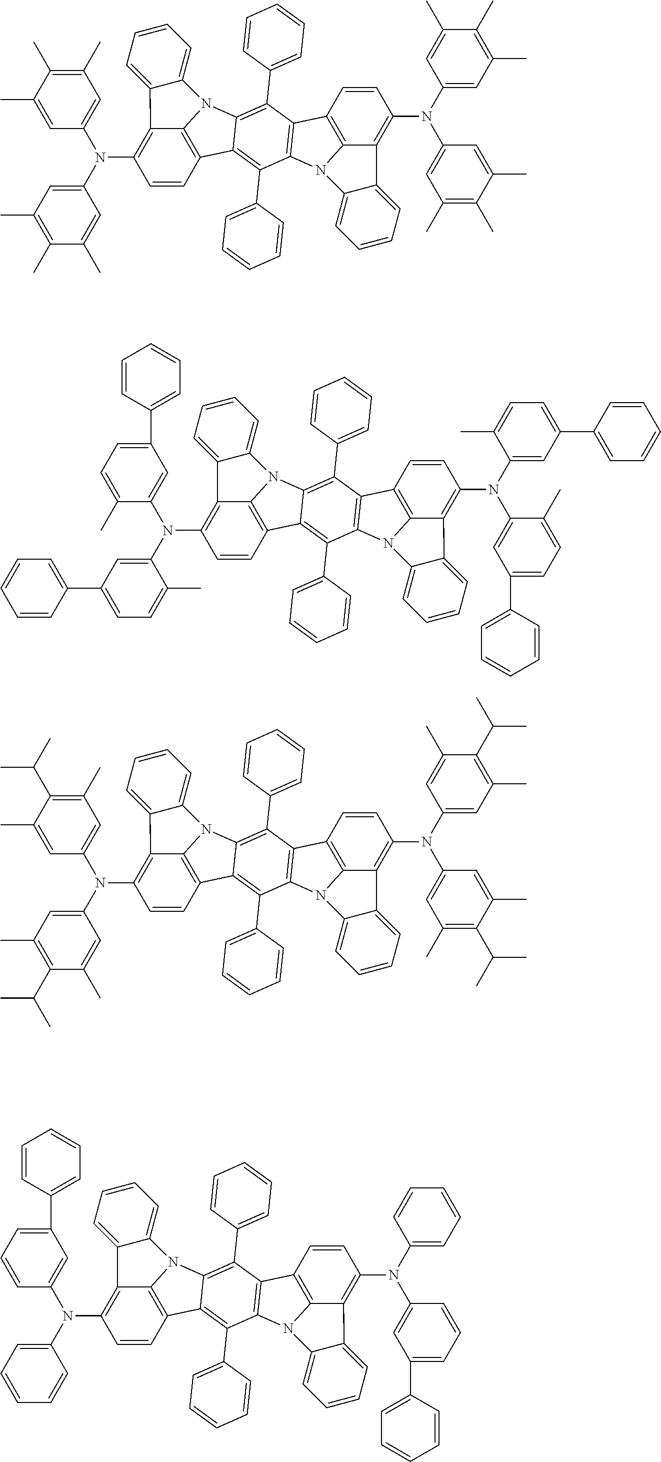

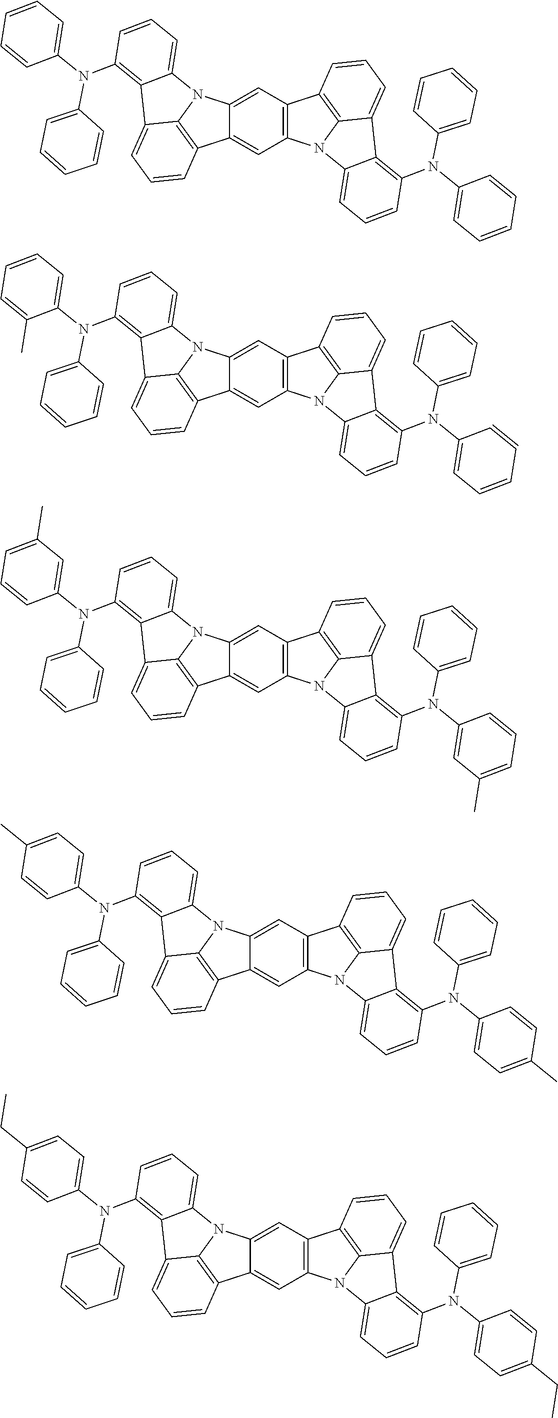

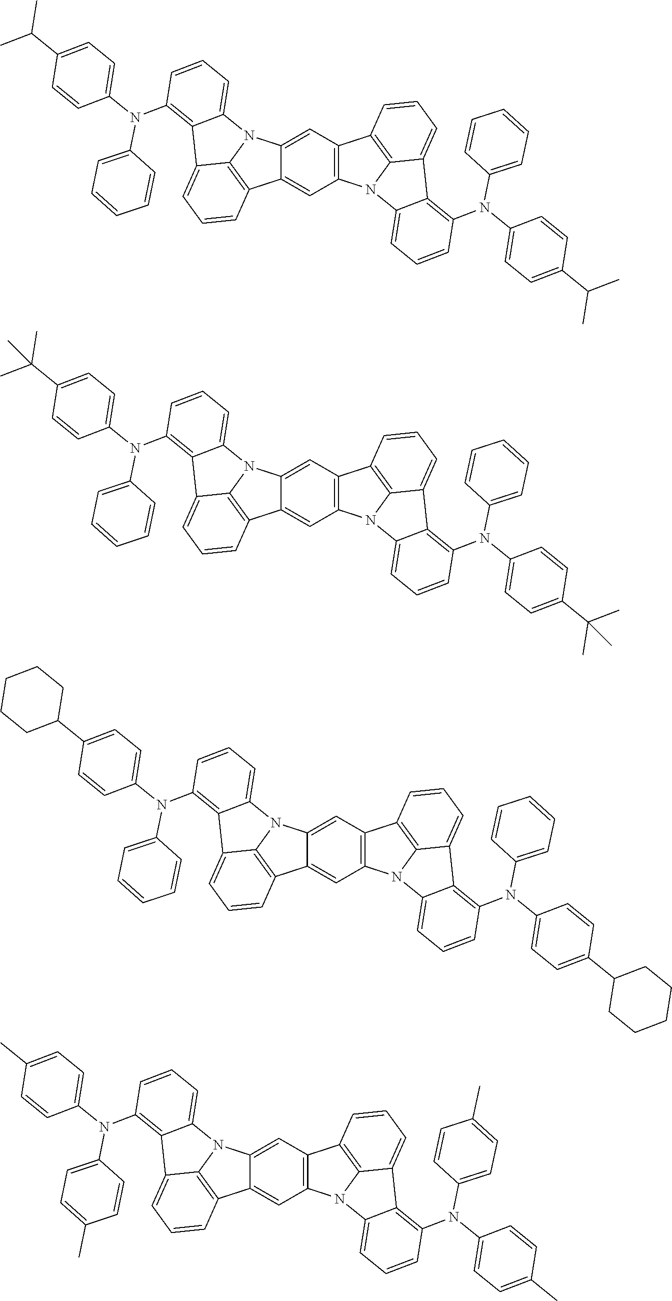

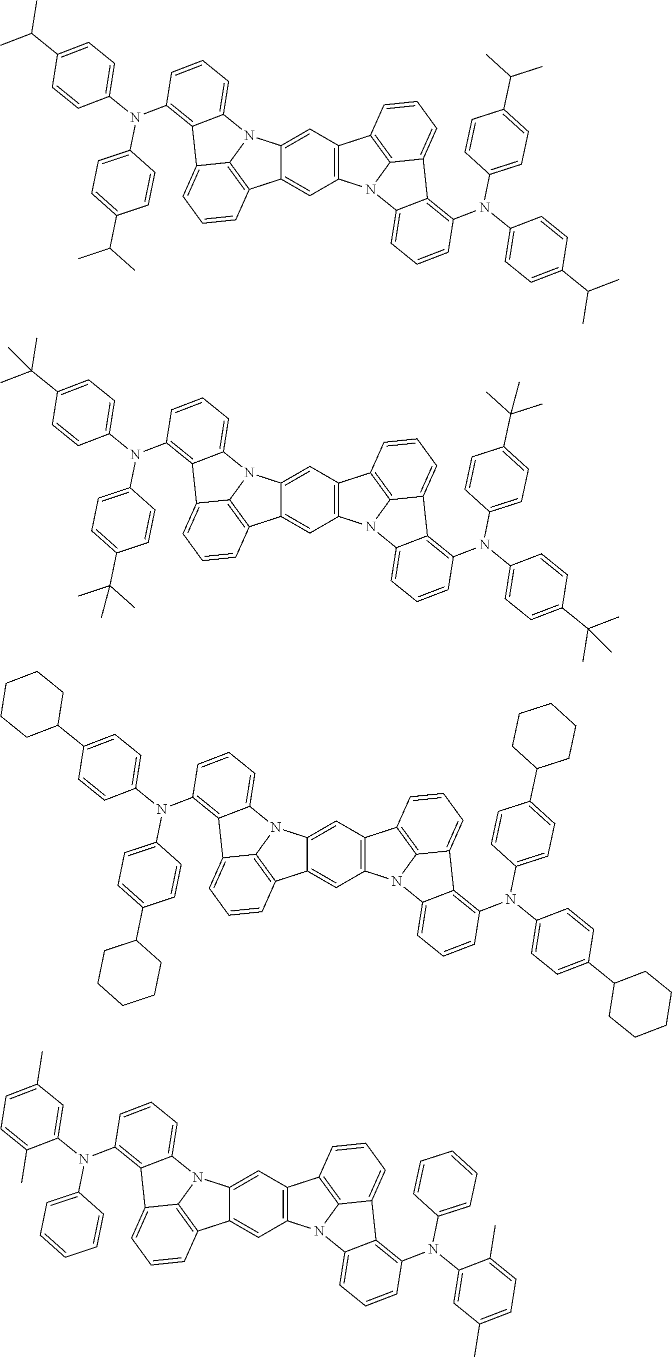

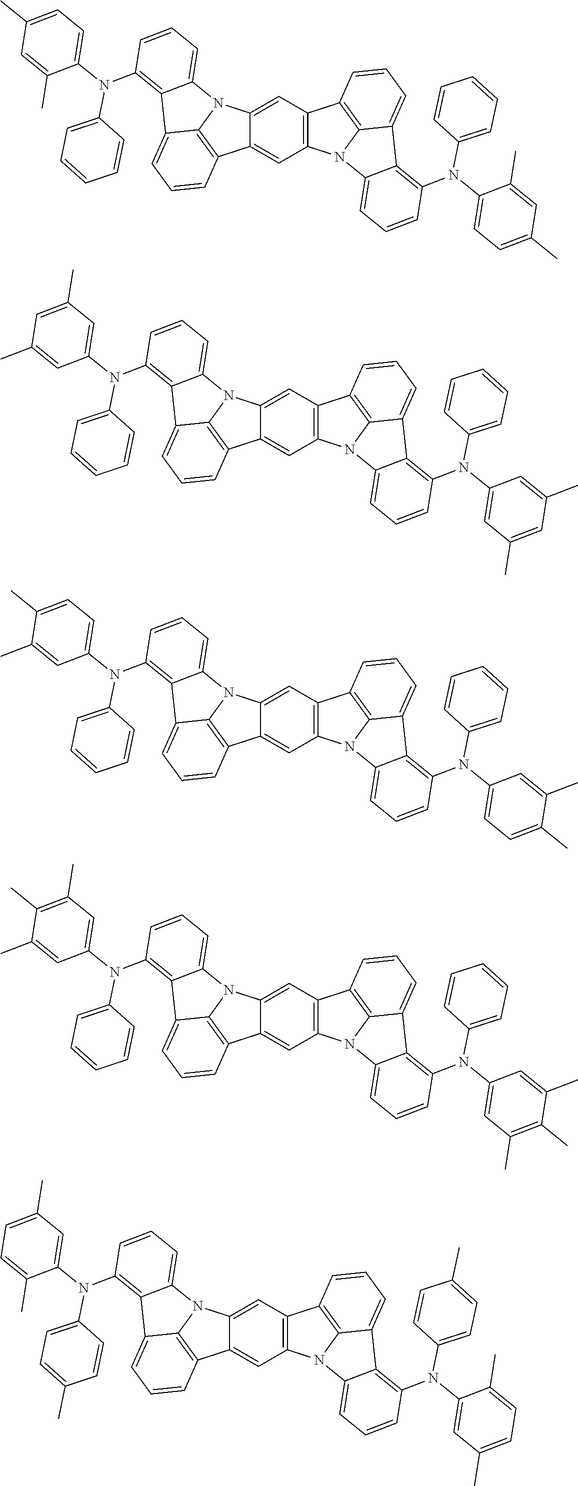

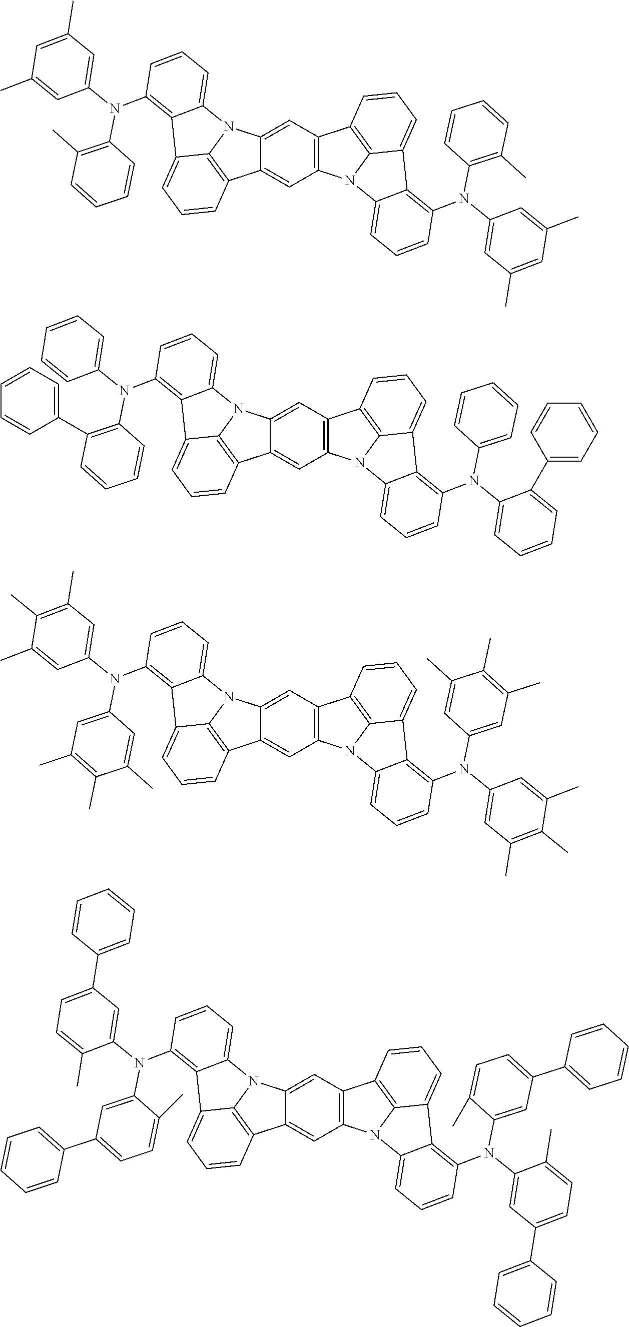

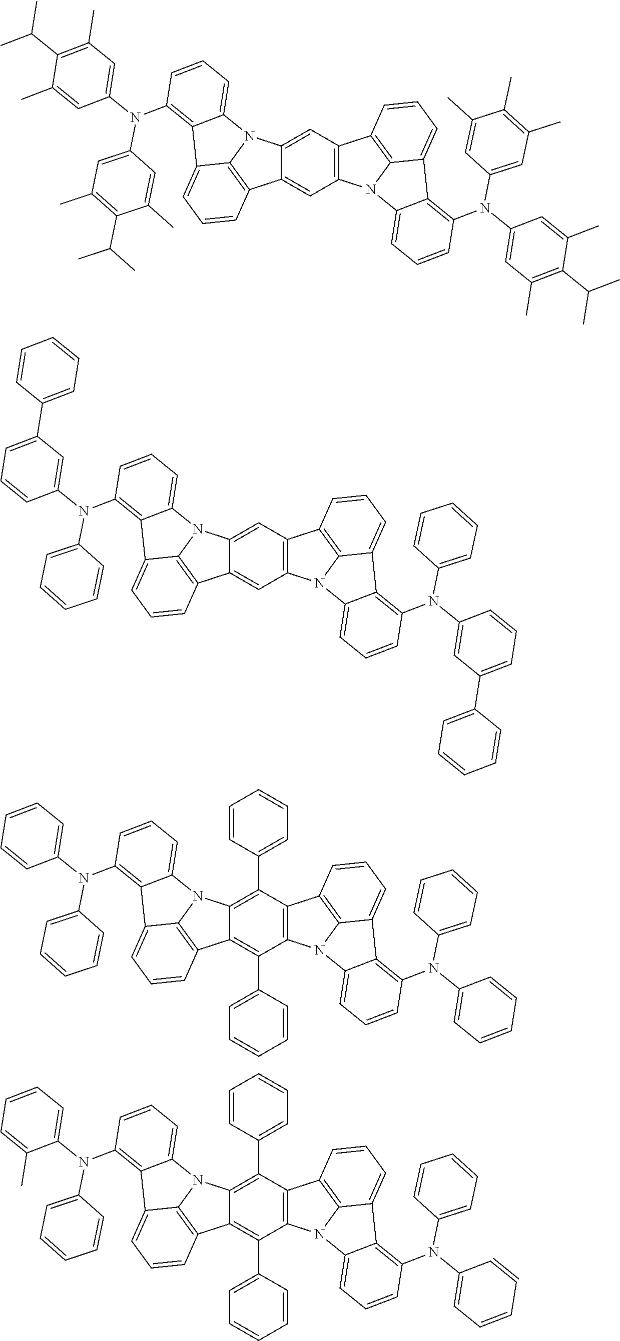

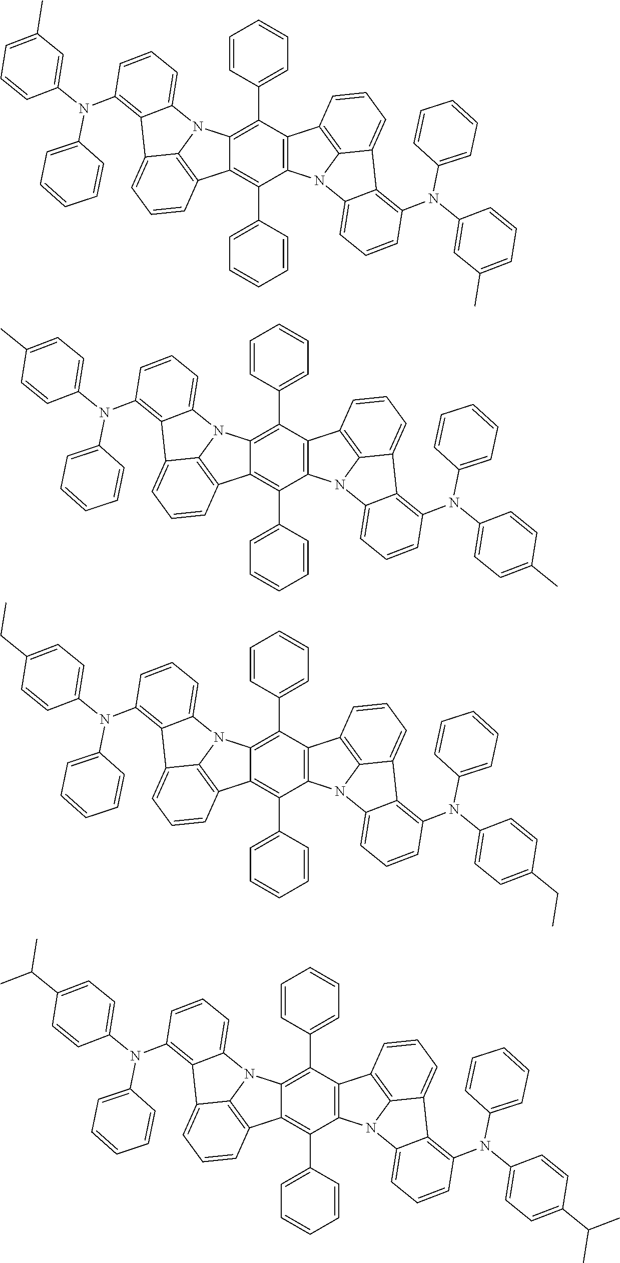

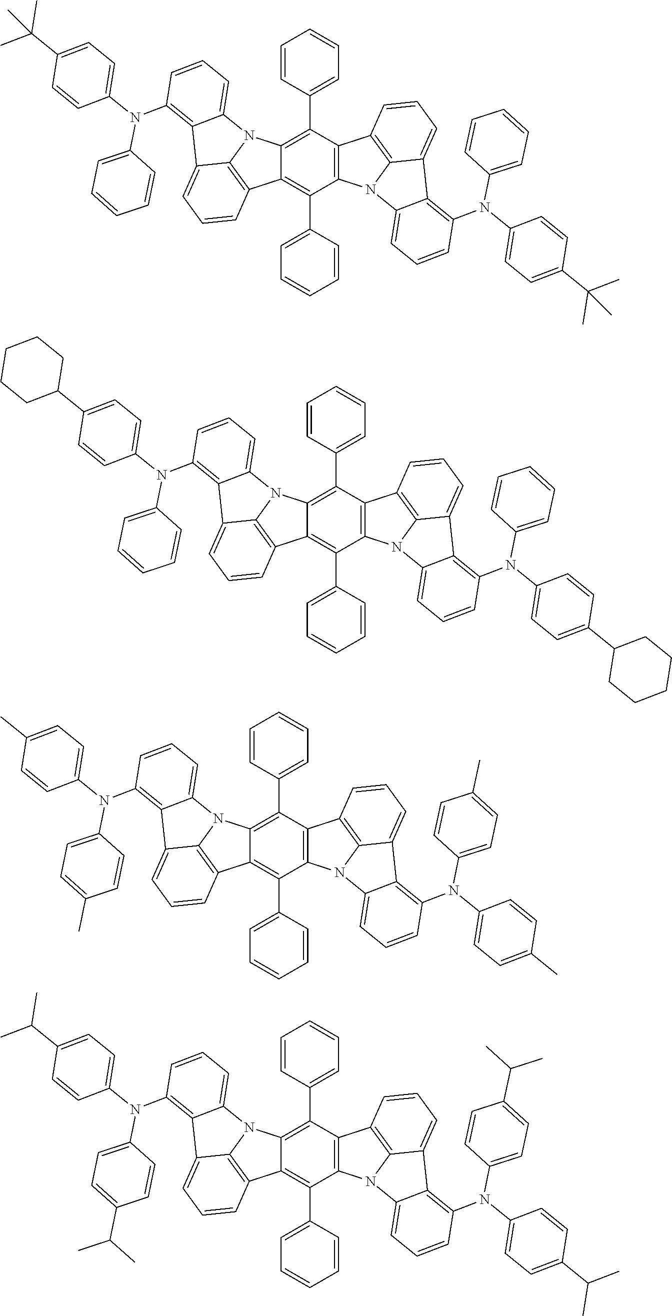

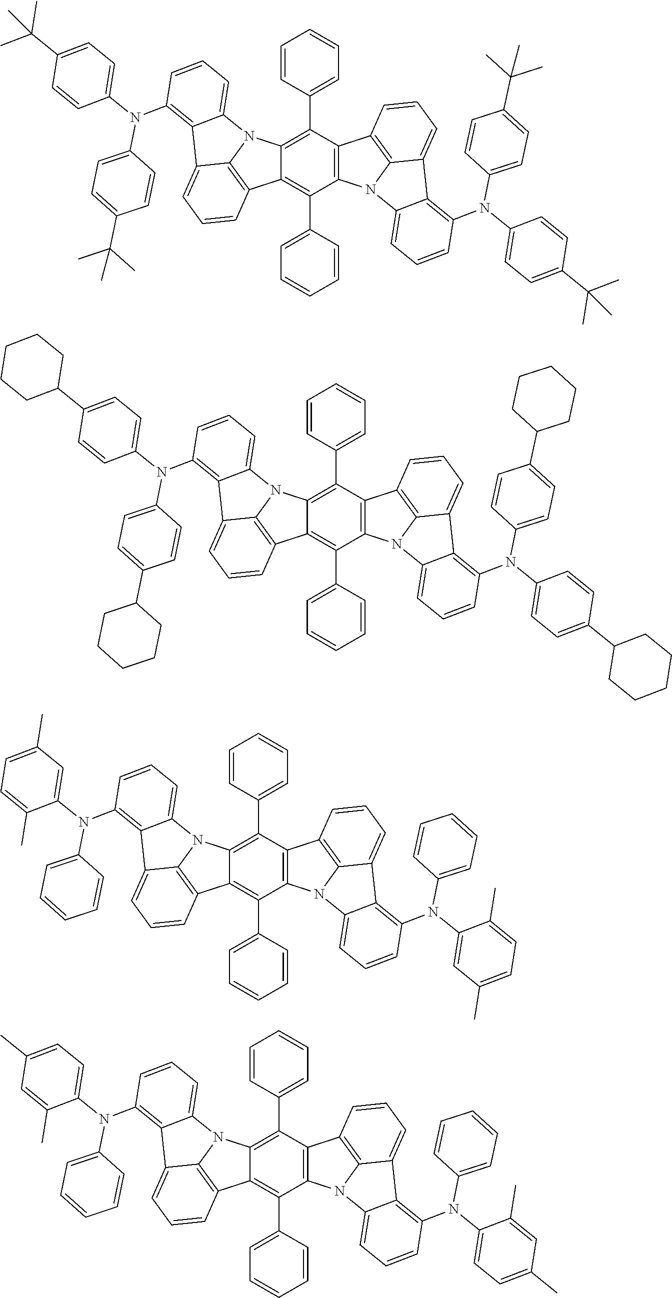

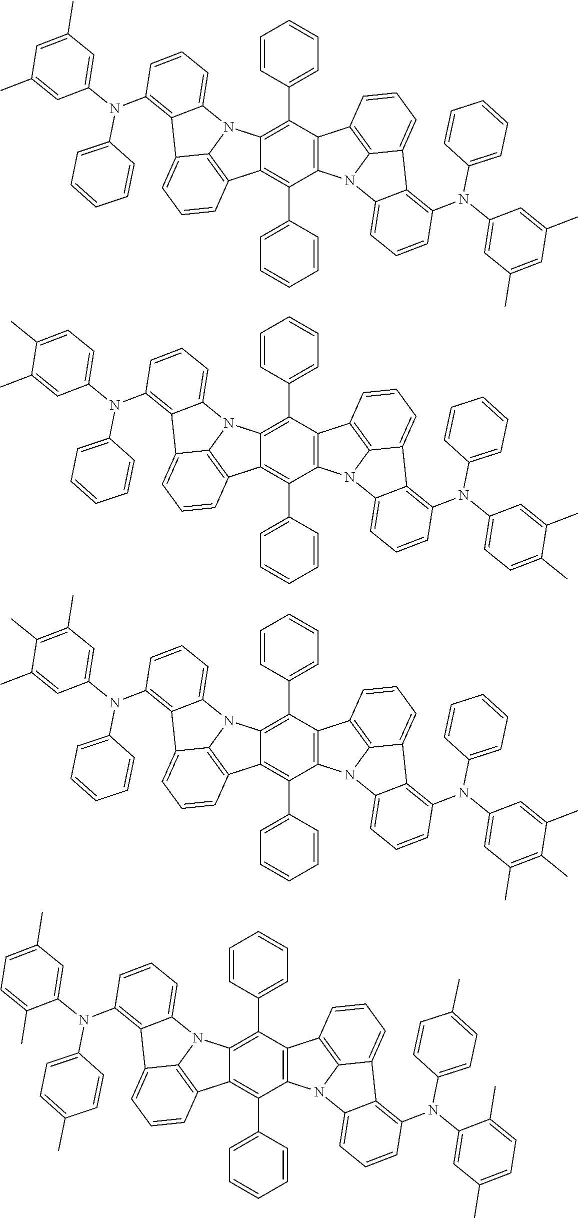

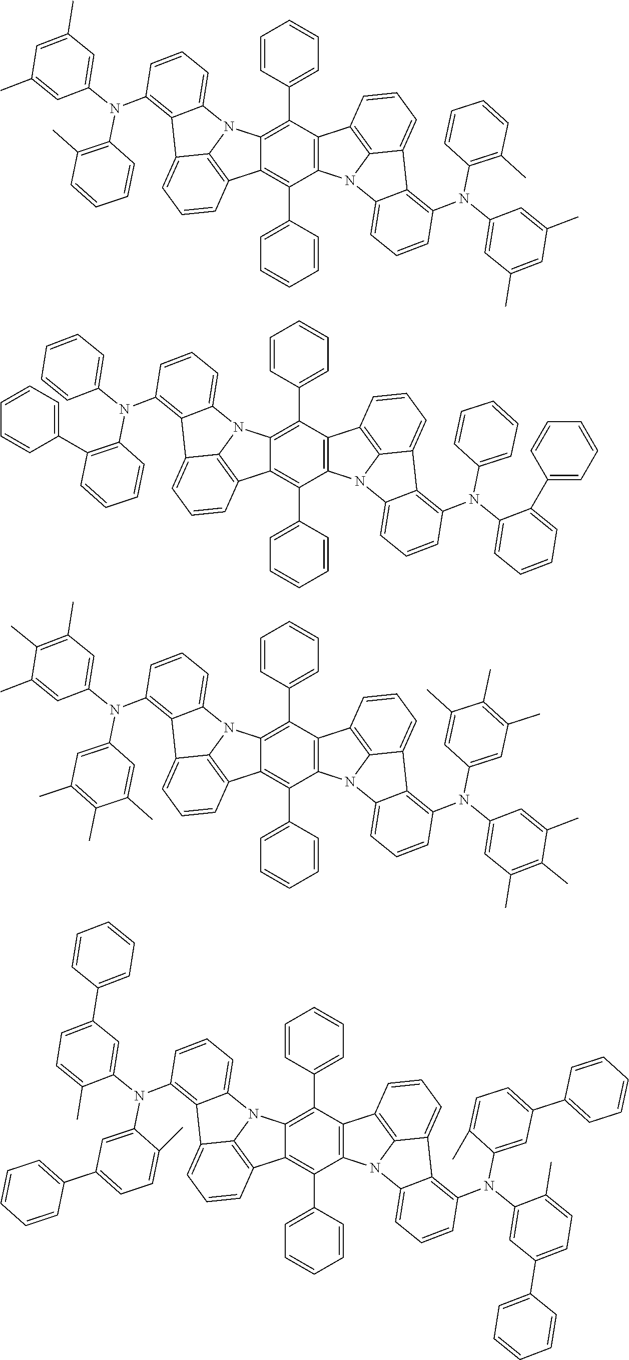

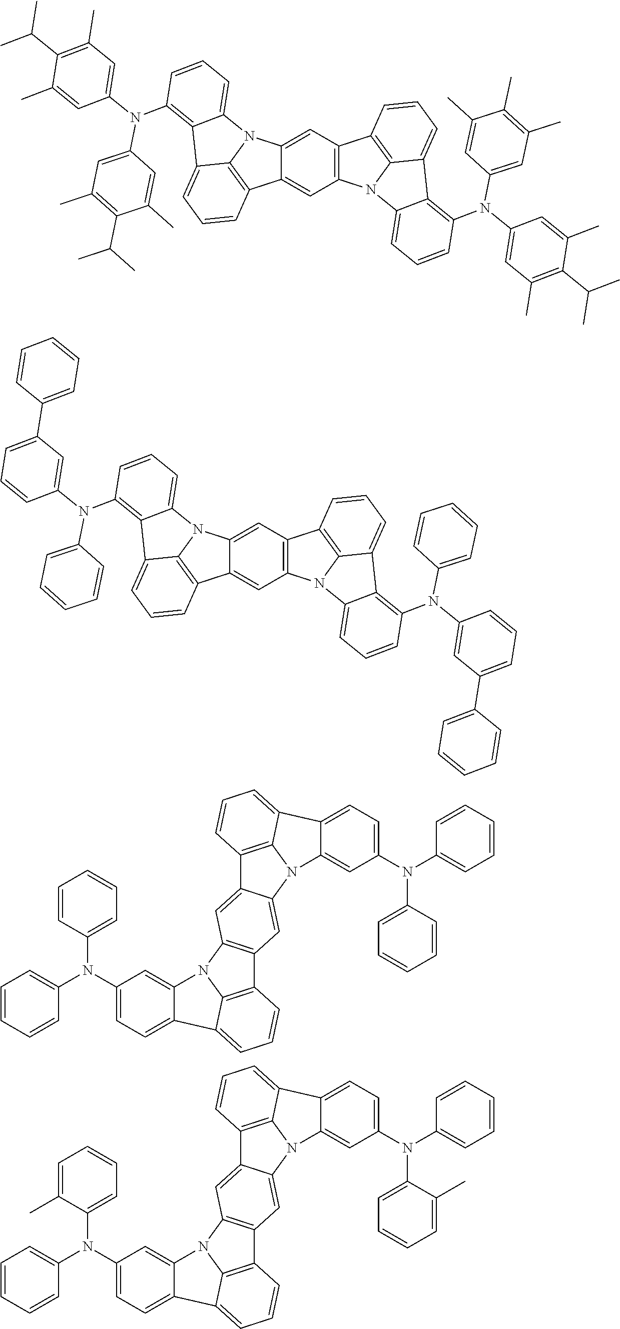

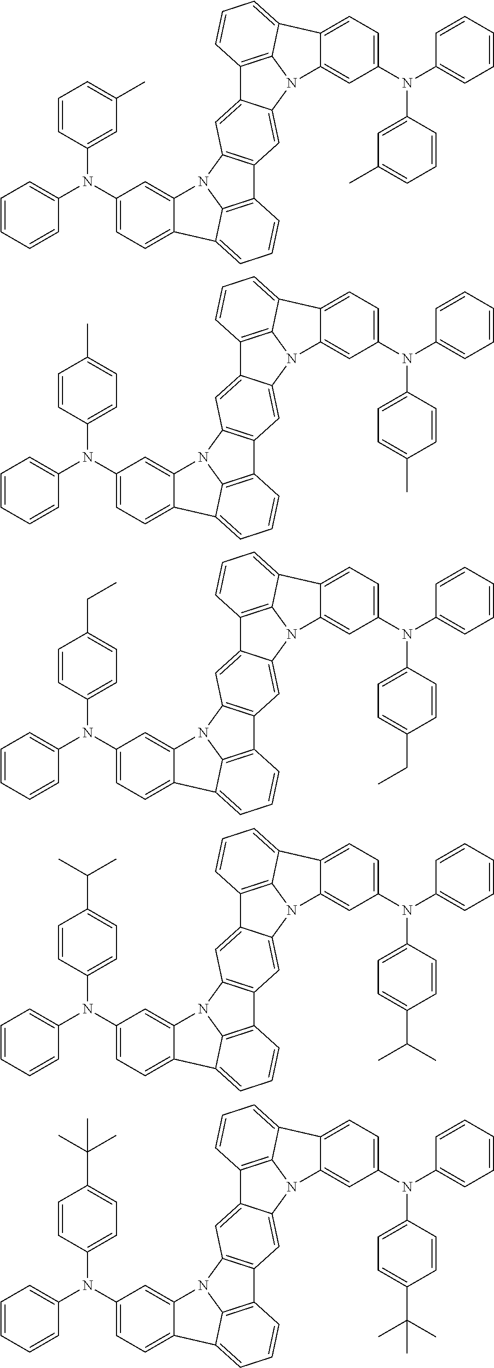

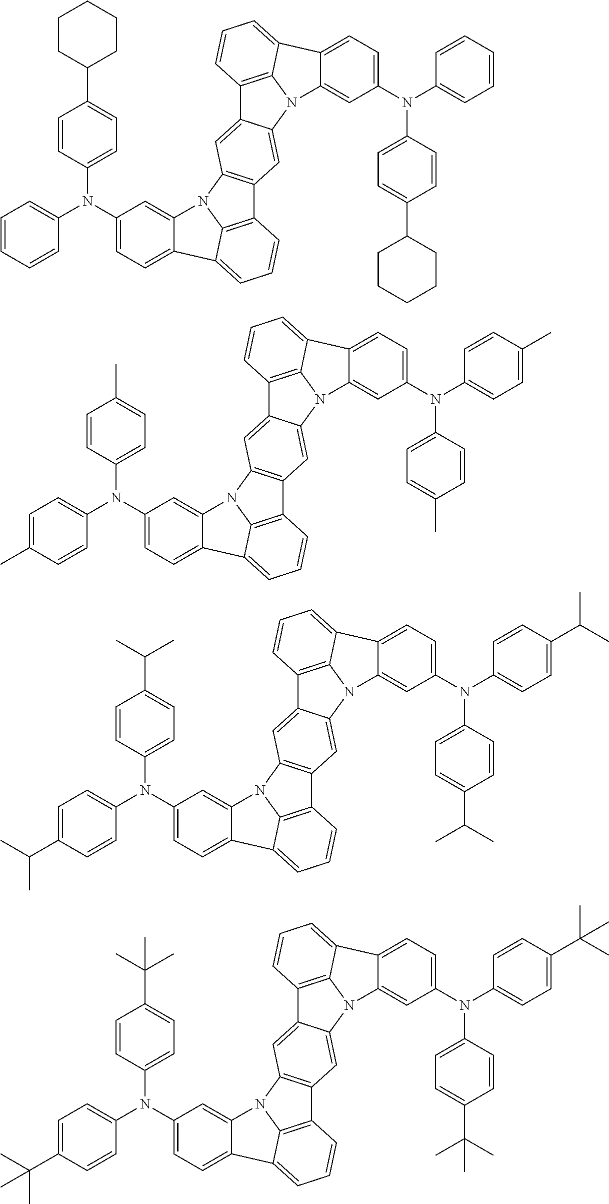

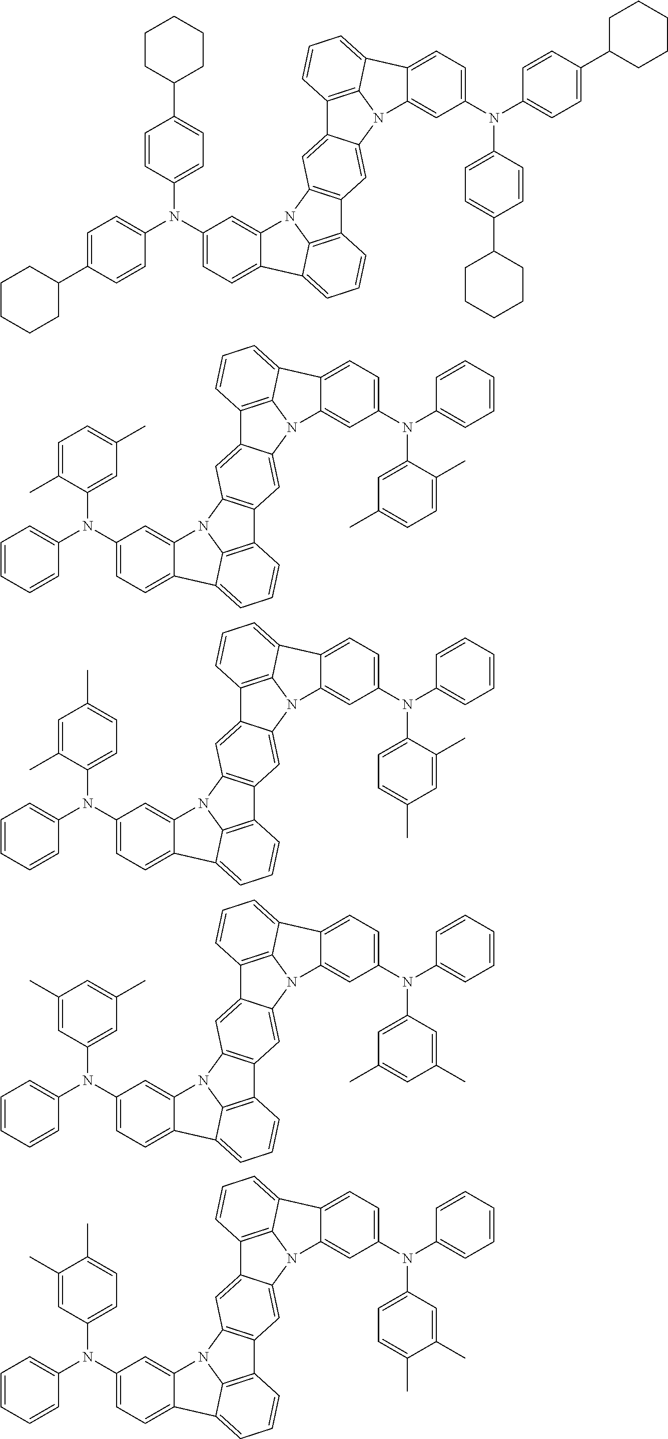

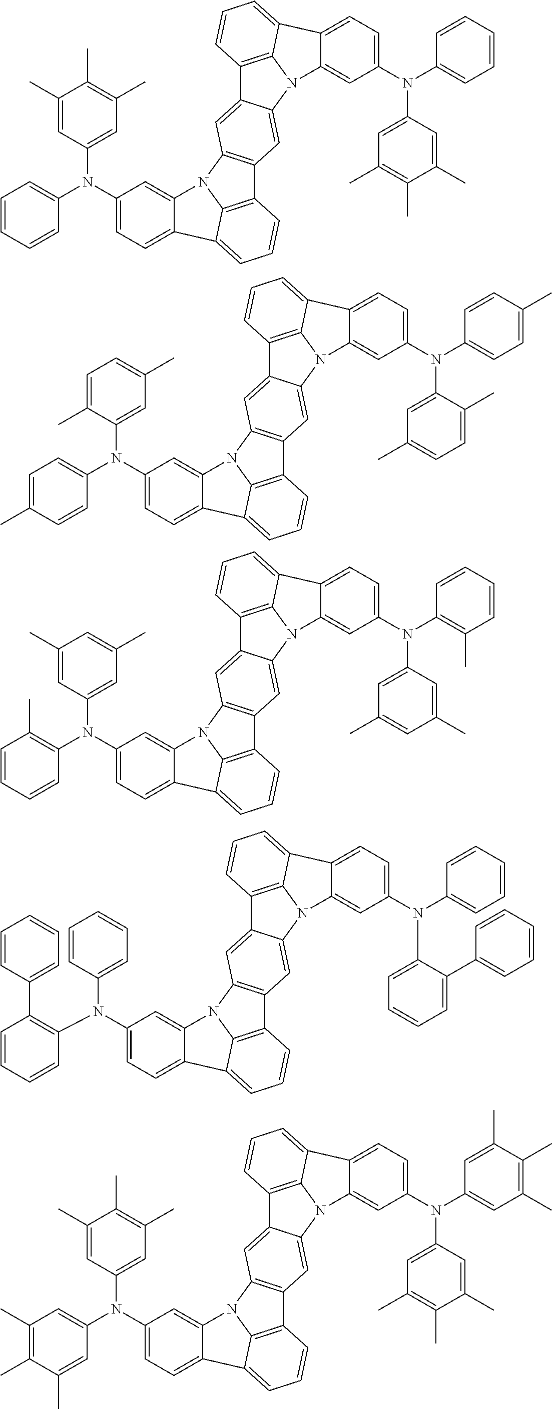

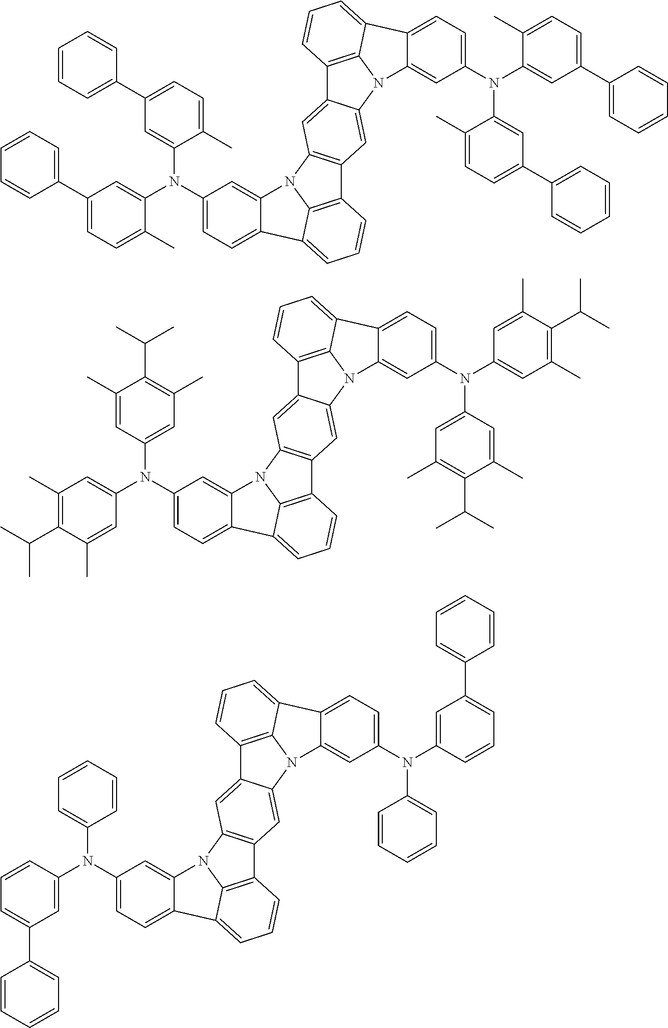

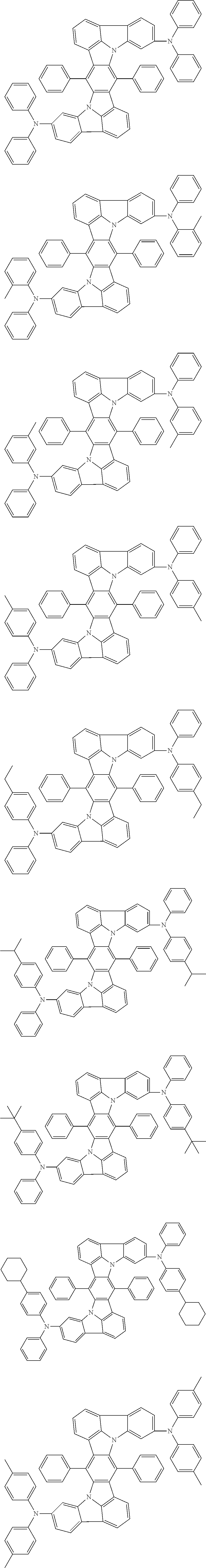

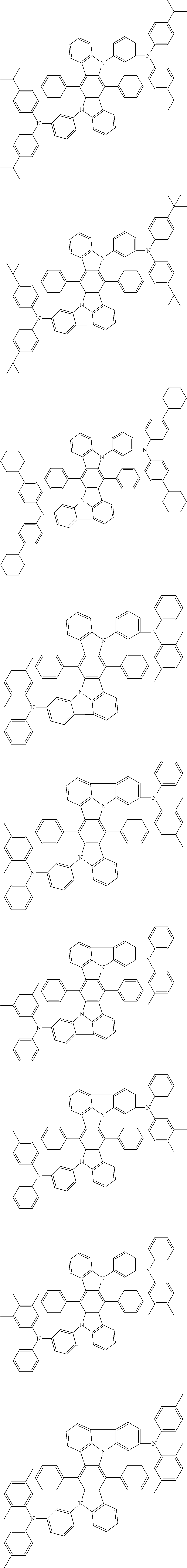

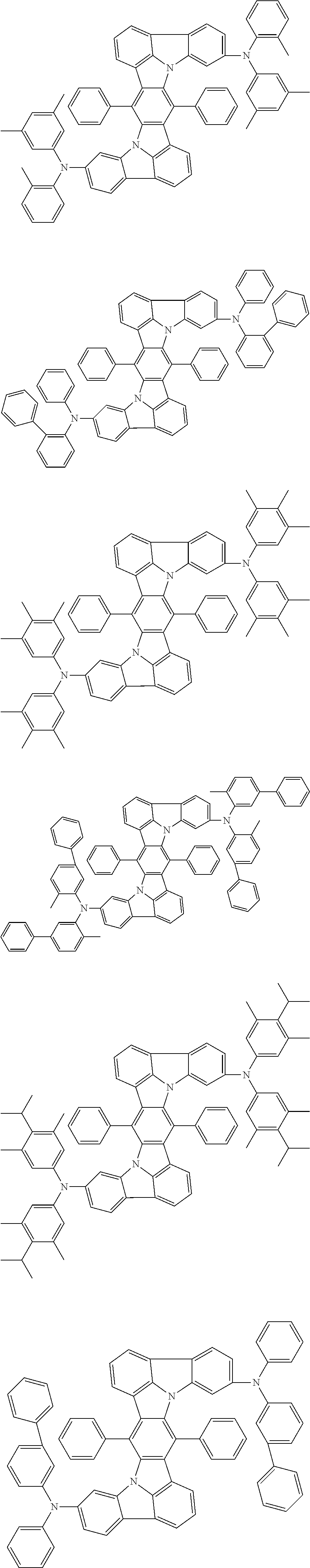

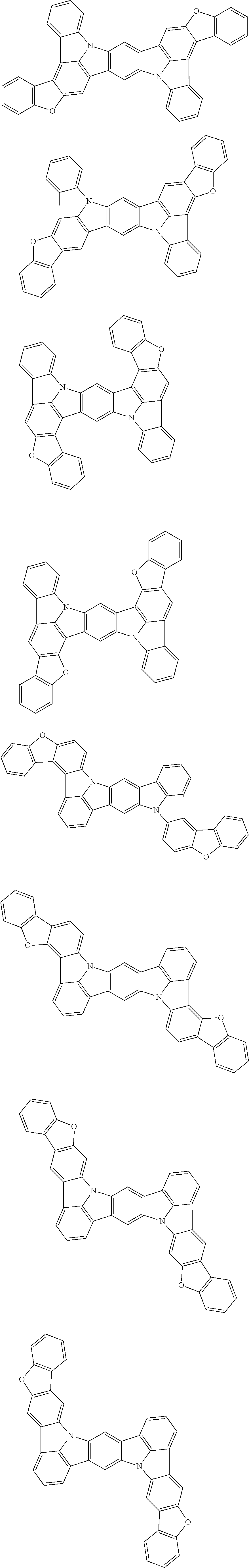

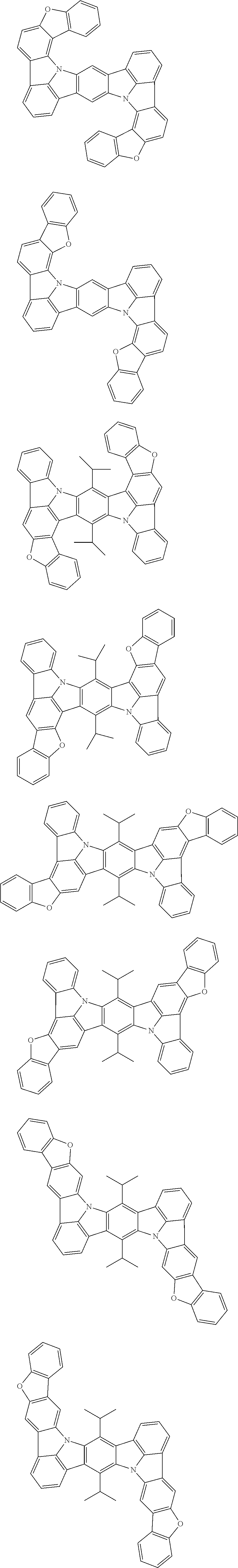

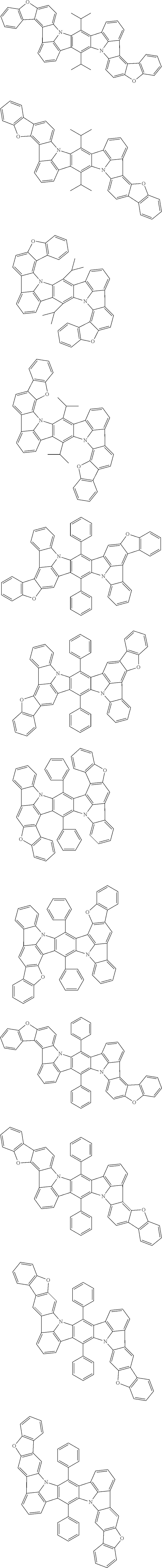

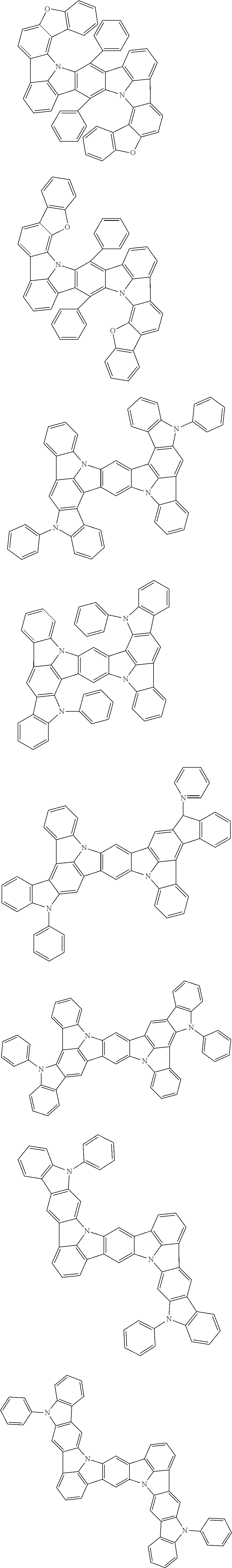

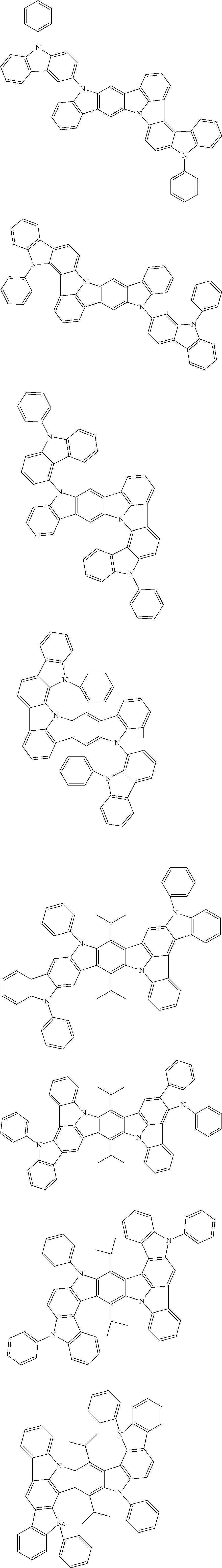

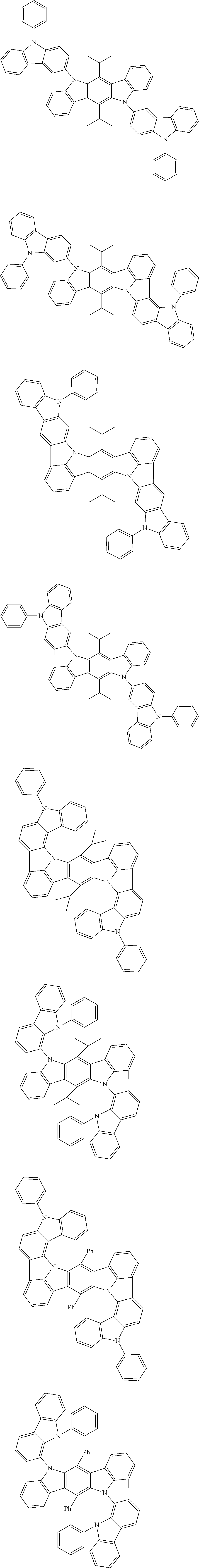

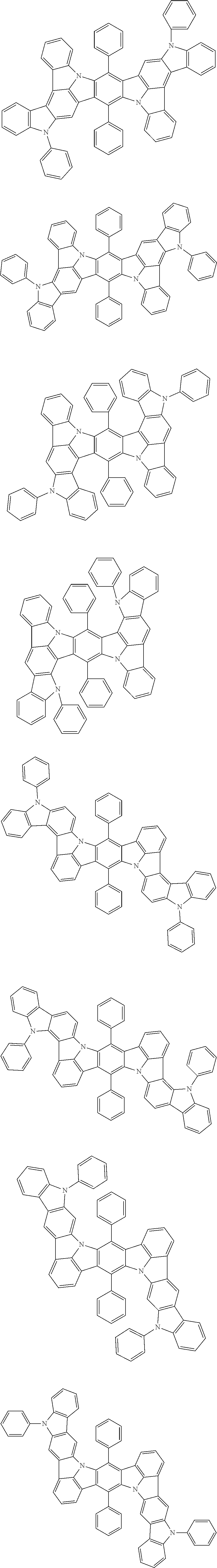

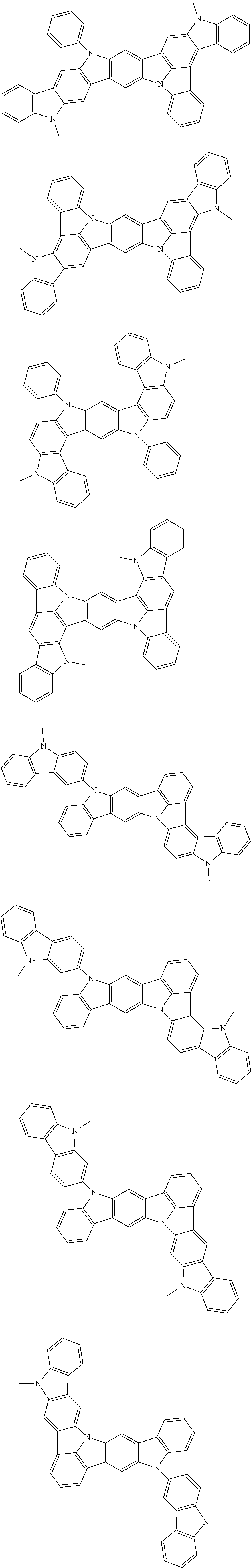

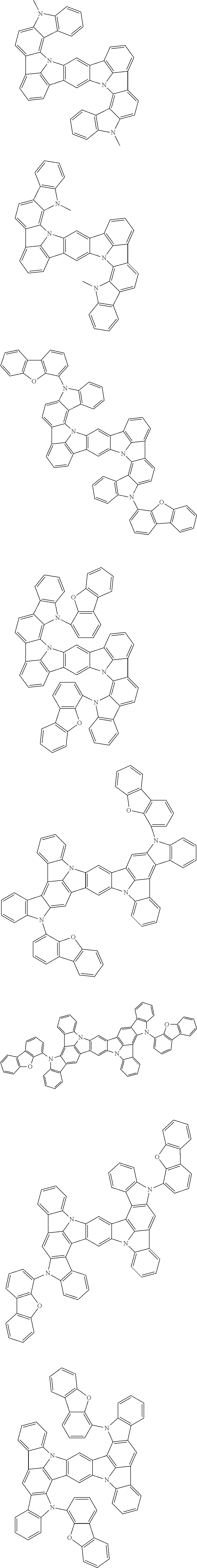

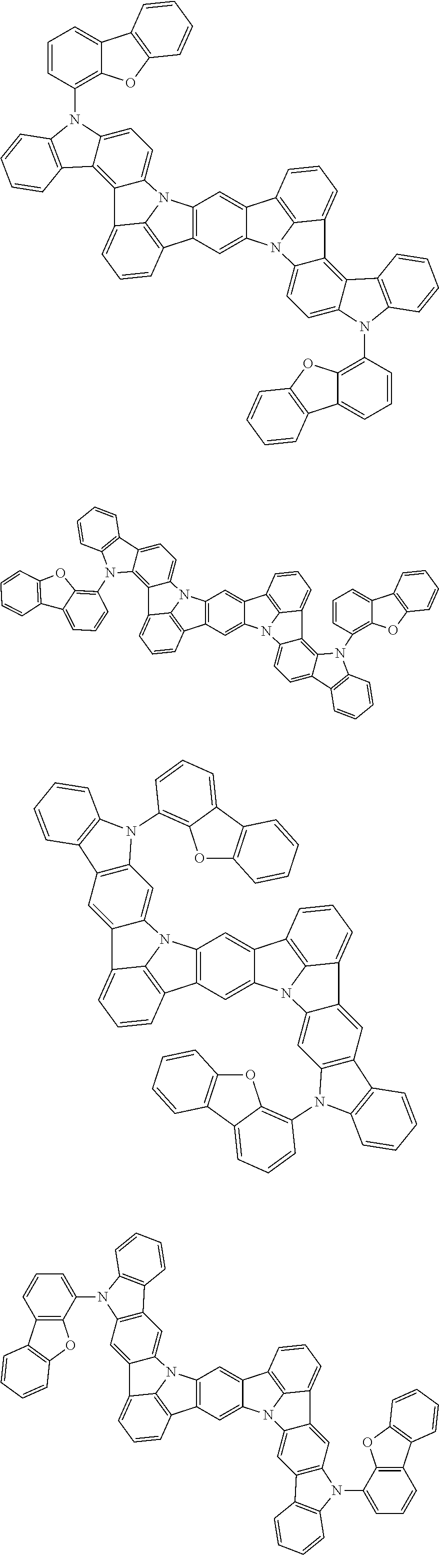 [SMILES: c1ccc2c(c1)oc1c(-n3c4ccccc4c4c5c6cccc7c8cc9c(cc8n(c5ccc43)c76)c3cccc4c5c6c7ccccc7n(-c7cccc8c7oc7ccccc78)c6ccc5n9c34)cccc12 ; c1ccc2c(c1)oc1c(-n3c4ccccc4c4ccc5c(c6cccc7c8cc9c(cc8n5c76)c5cccc6c7c8c(ccc7n9c56)c5ccccc5n8-c5cccc6c5oc5ccccc56)c43)cccc12 ; c1ccc2c(c1)oc1c(-n3c4ccccc4c4cc5c6cccc7c8cc9c(cc8n(c5cc43)c76)c3cccc4c5cc6c7ccccc7n(-c7cccc8c7oc7ccccc78)c6cc5n9c34)cccc12 ; c1ccc2c(c1)oc1c(-n3c4ccccc4c4cc5c(cc43)c3cccc4c6cc7c(cc6n5c34)c3cccc4c5cc6c(cc5n7c43)c3ccccc3n6-c3cccc4c3oc3ccccc34)cccc12]

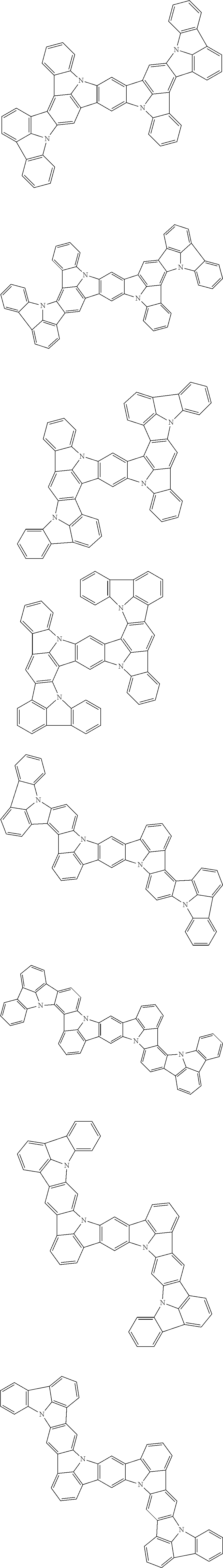

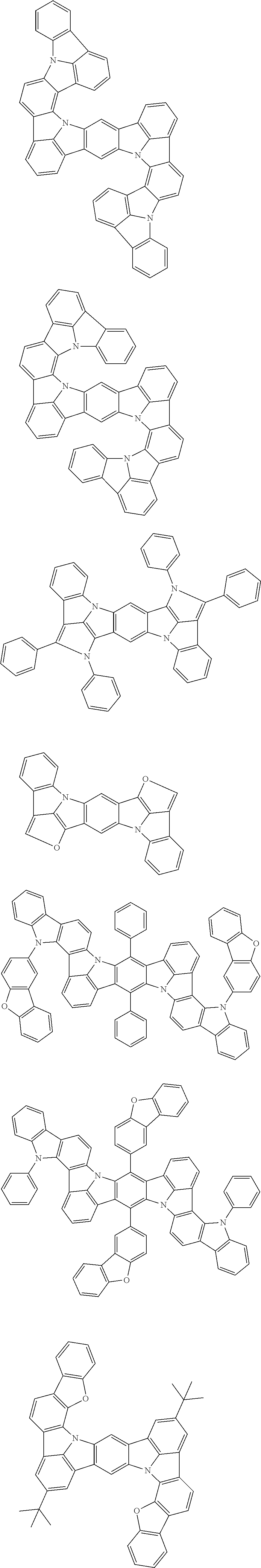

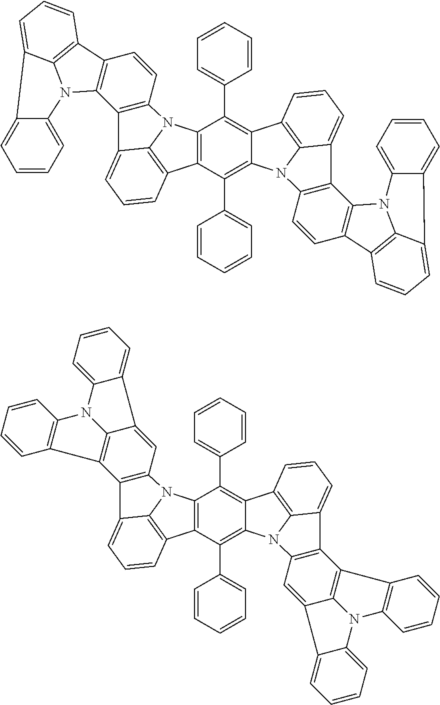 [SMILES: c1ccc(-c2c3c4cccc5c6c(ccc7c8cccc9c%10ccccc%10n(c98)c76)n(c3c(-c3ccccc3)c3c6cccc7c8c(ccc9c%10cccc%11c%12ccccc%12n(c%11%10)c98)n(c23)c67)c45)cc1 ; c1ccc(-c2c3c4cccc5c6c7c8ccccc8n8c9ccccc9c(cc6n(c3c(-c3ccccc3)c3c6cccc9c%10c%11c%12ccccc%12n%12c%13ccccc%13c(cc%10n(c23)c69)c%11%12)c45)c78)cc1]

### Compound Represented by Formula (6)

The compound represented by the formula (6) will be described below.

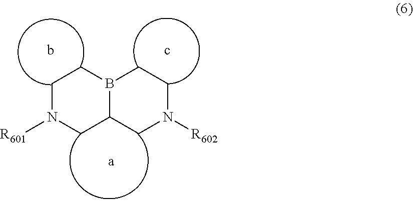

In the formula (6):
a ring a, a ring b and a ring c are each independently a substituted or unsubstituted aromatic hydrocarbon ring having 6 to 50 ring carbon atoms, or a substituted or unsubstituted heterocycle having 5 to 50 ring atoms;
R₆₀₁ and R₆₀₂ are each independently bonded to the ring a, ring b or ring c to form a substituted or unsubstituted heterocycle, or not bonded thereto to form no substituted or unsubstituted heterocycle; and
R₆₀₁ and R₆₀₂ not forming the substituted or unsubstituted heterocycle are each independently a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

### Specific Examples of Compound Represented by Formula (6)

Specific examples of the compound represented by the formula (6) are shown below. It should however be noted that these specific examples are merely exemplary and do not limit the compound represented by the formula (6).

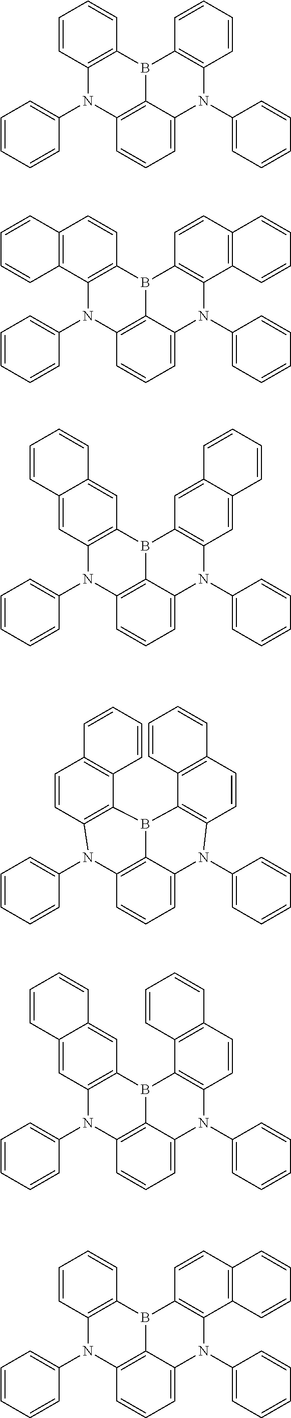 [SMILES: c1ccc(N2c3ccccc3B3c4ccccc4N(c4ccccc4)c4cccc2c43)cc1 ; c1ccc(N2c3cccc4c3B(c3ccc5ccccc5c32)c2ccc3ccccc3c2N4c2ccccc2)cc1 ; c1ccc(N2c3cc4ccccc4cc3B3c4cc5ccccc5cc4N(c4ccccc4)c4cccc2c43)cc1 ; c1ccc(N2c3cccc4c3B(c3c2ccc2ccccc32)c2c(ccc3ccccc23)N4c2ccccc2)cc1 ; c1ccc(N2c3cc4ccccc4cc3B3c4c2cccc4N(c2ccccc2)c2ccc4ccccc4c23)cc1 ; c1ccc(N2c3ccccc3B3c4ccc5ccccc5c4N(c4ccccc4)c4cccc2c43)cc1]

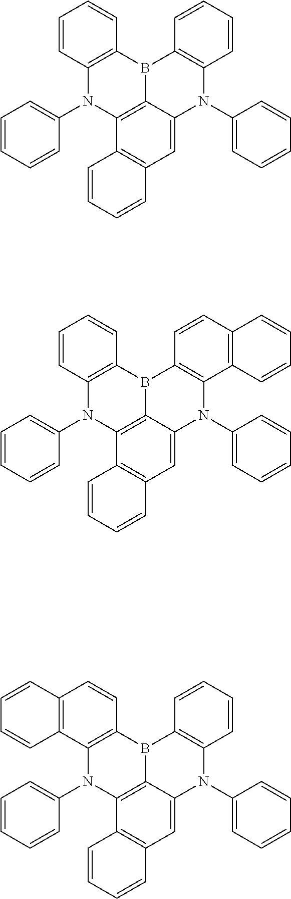 [SMILES: c1ccc(N2c3ccccc3B3c4ccccc4N(c4ccccc4)c4c3c2cc2ccccc42)cc1 ; c1ccc(N2c3cc4ccccc4c4c3B(c3ccccc3N4c3ccccc3)c3ccc4ccccc4c32)cc1 ; c1ccc(N2c3ccccc3B3c4ccc5ccccc5c4N(c4ccccc4)c4c3c2cc2ccccc42)cc1]

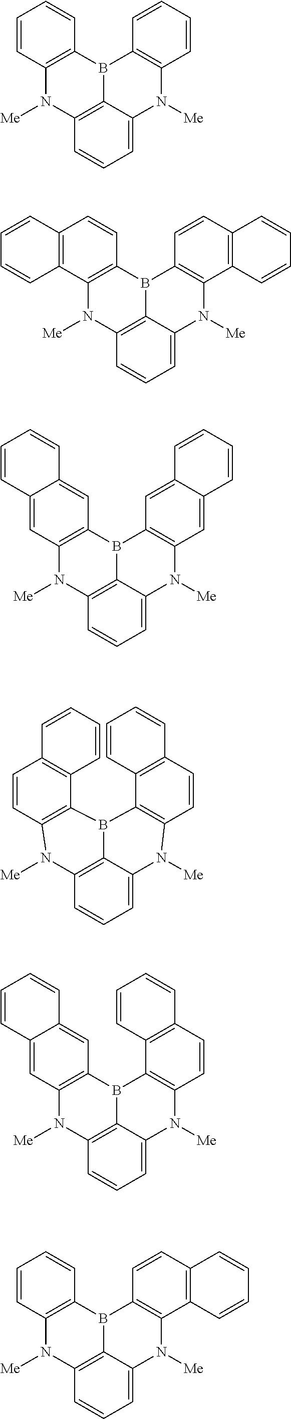 [SMILES: CN1c2ccccc2B2c3ccccc3N(C)c3cccc1c32 ; CN1c2cccc3c2B(c2ccc4ccccc4c21)c1ccc2ccccc2c1N3C ; CN1c2cc3ccccc3cc2B2c3cc4ccccc4cc3N(C)c3cccc1c32 ; CN1c2cccc3c2B(c2c1ccc1ccccc21)c1c(ccc2ccccc12)N3C ; CN1c2cc3ccccc3cc2B2c3c1cccc3N(C)c1ccc3ccccc3c12 ; CN1c2ccccc2B2c3ccc4ccccc4c3N(C)c3cccc1c32]

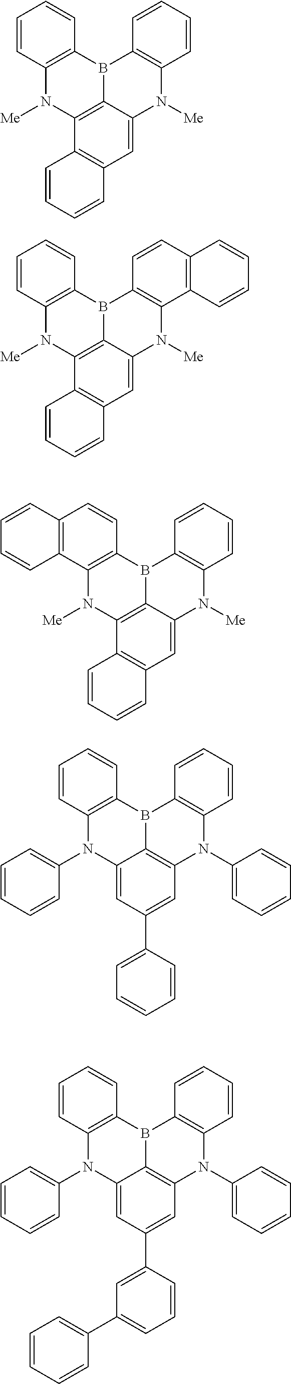

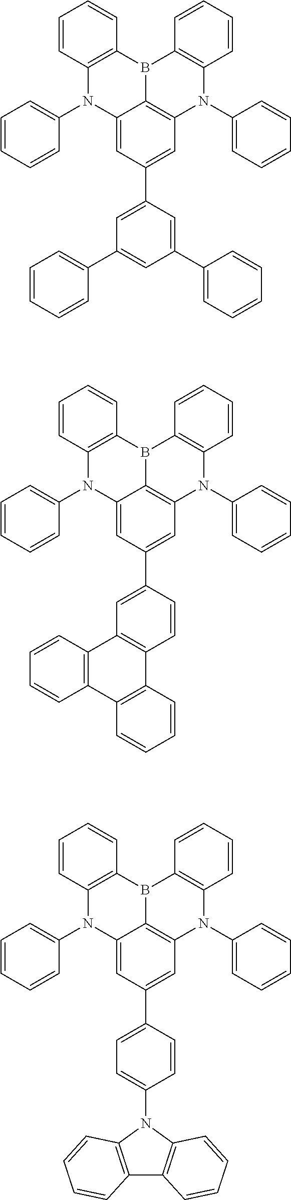 [SMILES: c1ccc(-c2cc(-c3ccccc3)cc(-c3cc4c5c(c3)N(c3ccccc3)c3ccccc3B5c3ccccc3N4c3ccccc3)c2)cc1 ; c1ccc(N2c3ccccc3B3c4ccccc4N(c4ccccc4)c4cc(-c5ccc6c7ccccc7c7ccccc7c6c5)cc2c43)cc1 ; c1ccc(N2c3ccccc3B3c4ccccc4N(c4ccccc4)c4cc(-c5ccc(-n6c7ccccc7c7ccccc76)cc5)cc2c43)cc1]

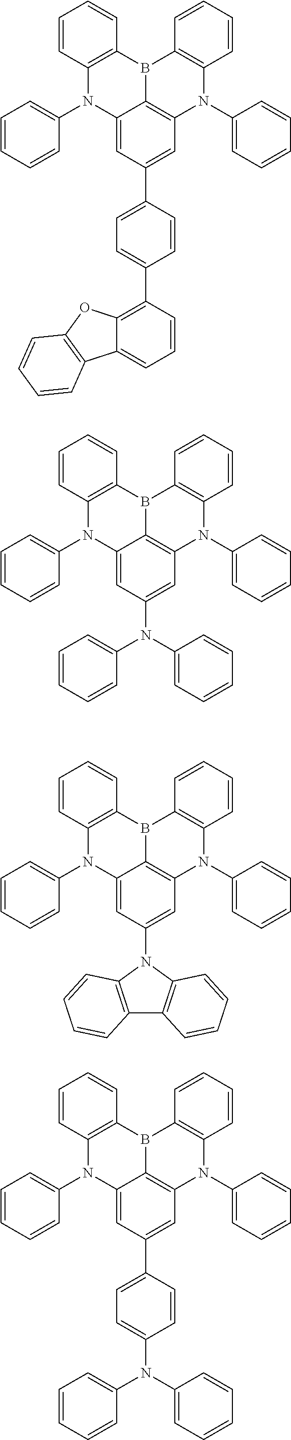

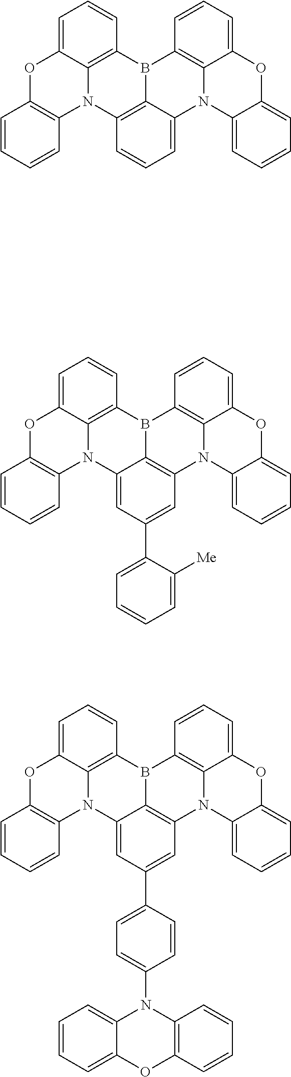 [SMILES: c1ccc2c(c1)Oc1cccc3c1N2c1cccc2c1B3c1cccc3c1N2c1ccccc1O3 ; Cc1ccccc1-c1cc2c3c(c1)N1c4ccccc4Oc4cccc(c41)B3c1cccc3c1N2c1ccccc1O3 ; c1ccc2c(c1)Oc1ccccc1N2c1ccc(-c2cc3c4c(c2)N2c5ccccc5Oc5cccc(c52)B4c2cccc4c2N3c2ccccc2O4)cc1]

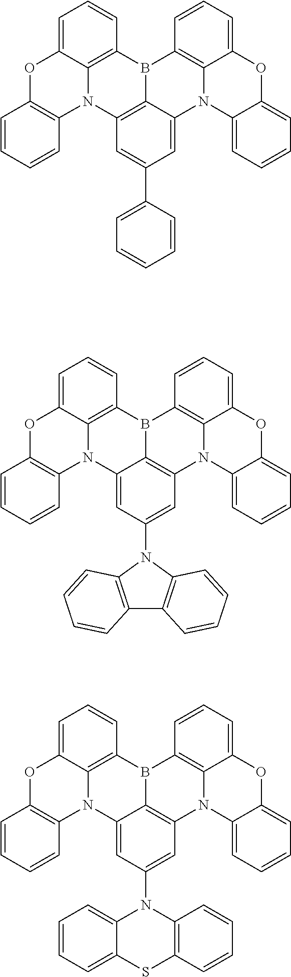 [SMILES: c1ccc(-c2cc3c4c(c2)N2c5ccccc5Oc5cccc(c52)B4c2cccc4c2N3c2ccccc2O4)cc1 ; c1ccc2c(c1)Oc1cccc3c1N2c1cc(-n2c4ccccc4c4ccccc42)cc2c1B3c1cccc3c1N2c1ccccc1O3 ; c1ccc2c(c1)Oc1cccc3c1N2c1cc(N2c4ccccc4Sc4ccccc42)cc2c1B3c1cccc3c1N2c1ccccc1O3]

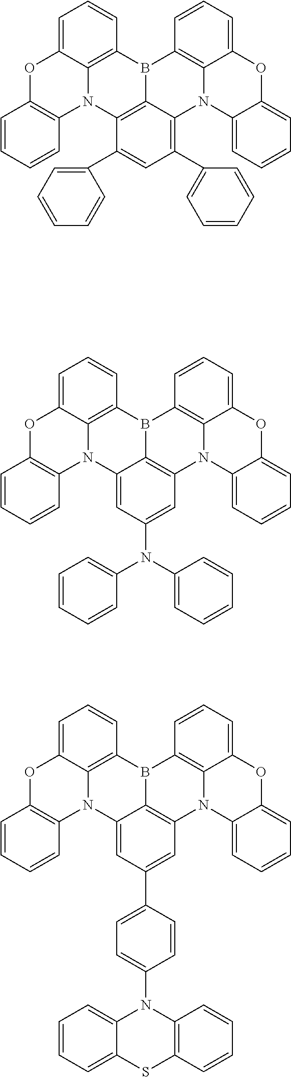 [SMILES: c1ccc(-c2cc(-c3ccccc3)c3c4c2N2c5ccccc5Oc5cccc(c52)B4c2cccc4c2N3c2ccccc2O4)cc1 ; c1ccc(N(c2ccccc2)c2cc3c4c(c2)N2c5ccccc5Oc5cccc(c52)B4c2cccc4c2N3c2ccccc2O4)cc1 ; c1ccc2c(c1)Oc1cccc3c1N2c1cc(-c2ccc(N4c5ccccc5Sc5ccccc54)cc2)cc2c1B3c1cccc3c1N2c1ccccc1O3]

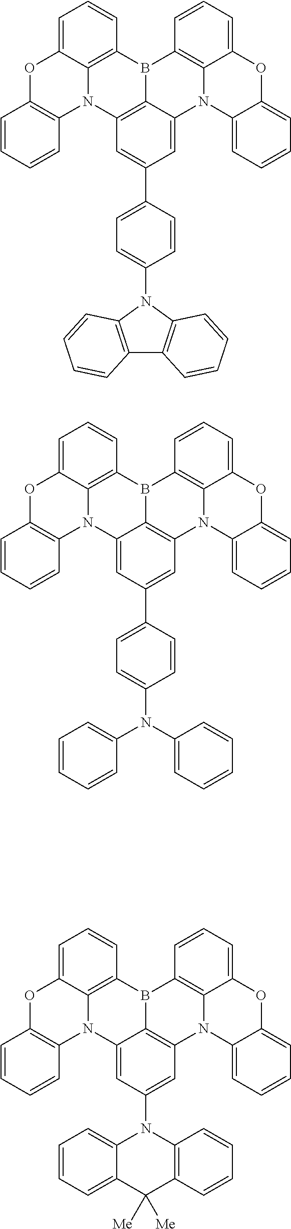 [SMILES: c1ccc2c(c1)Oc1cccc3c1N2c1cc(-c2ccc(-n4c5ccccc5c5ccccc54)cc2)cc2c1B3c1cccc3c1N2c1ccccc1O3 ; c1ccc(N(c2ccccc2)c2ccc(-c3cc4c5c(c3)N3c6ccccc6Oc6cccc(c63)B5c3cccc5c3N4c3ccccc3O5)cc2)cc1 ; CC1(C)c2ccccc2N(c2cc3c4c(c2)N2c5ccccc5Oc5cccc(c52)B4c2cccc4c2N3c2ccccc2O4)c2ccccc21]

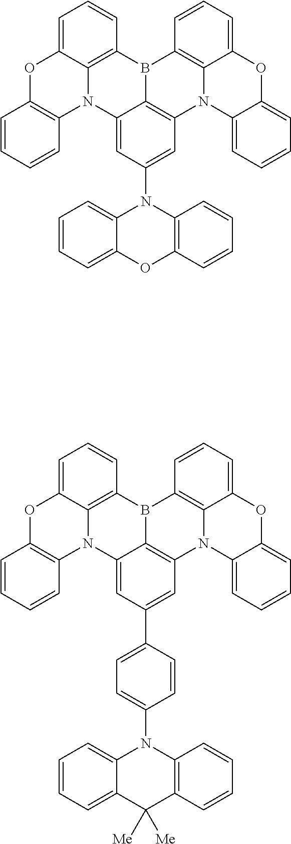 [SMILES: c1ccc2c(c1)Oc1ccccc1N2c1cc2c3c(c1)N1c4ccccc4Oc4cccc(c41)B3c1cccc3c1N2c1ccccc1O3 ; CC1(C)c2ccccc2N(c2ccc(-c3cc4c5c(c3)N3c6ccccc6Oc6cccc(c63)B5c3cccc5c3N4c3ccccc3O5)cc2)c2ccccc21]

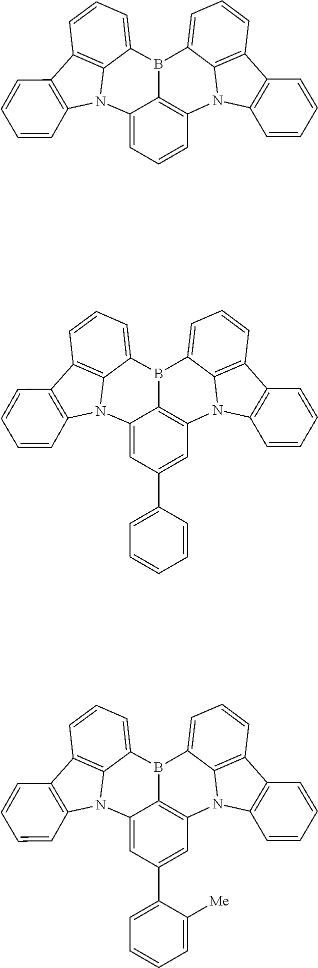 [SMILES: c1cc2c3c(c1)-n1c4ccccc4c4cccc(c41)B3c1cccc3c4ccccc4n-2c13 ; c1ccc(-c2cc3c4c(c2)-n2c5ccccc5c5cccc(c52)B4c2cccc4c5ccccc5n-3c24)cc1 ; Cc1ccccc1-c1cc2c3c(c1)-n1c4ccccc4c4cccc(c41)B3c1cccc3c4ccccc4n-2c13]

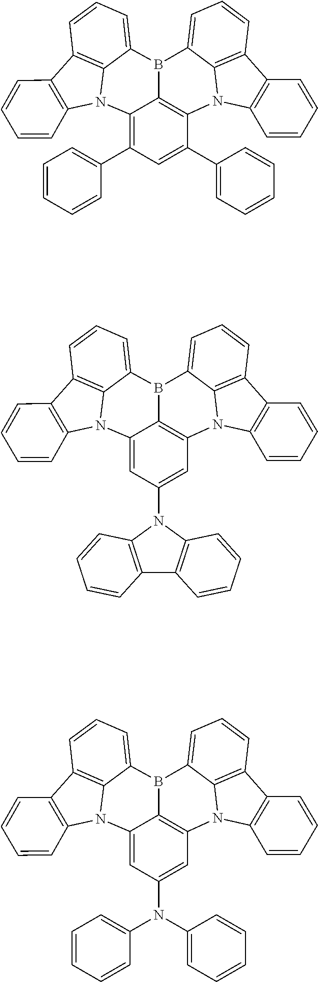 [SMILES: c1ccc(-c2cc(-c3ccccc3)c3c4c2-n2c5ccccc5c5cccc(c52)B4c2cccc4c5ccccc5n-3c24)cc1 ; c1ccc2c(c1)c1ccccc1n2-c1cc2c3c(c1)-n1c4ccccc4c4cccc(c41)B3c1cccc3c4ccccc4n-2c13 ; c1ccc(N(c2ccccc2)c2cc3c4c(c2)-n2c5ccccc5c5cccc(c52)B4c2cccc4c5ccccc5n-3c24)cc1]

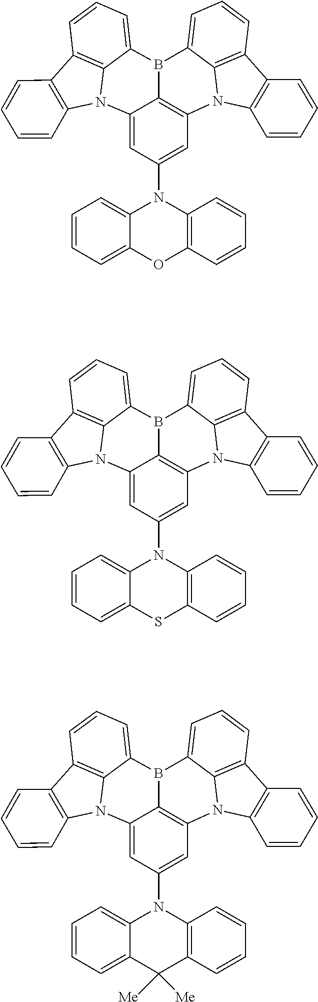 [SMILES: c1ccc2c(c1)Oc1ccccc1N2c1cc2c3c(c1)-n1c4ccccc4c4cccc(c41)B3c1cccc3c4ccccc4n-2c13 ; c1ccc2c(c1)Sc1ccccc1N2c1cc2c3c(c1)-n1c4ccccc4c4cccc(c41)B3c1cccc3c4ccccc4n-2c13 ; CC1(C)c2ccccc2N(c2cc3c4c(c2)-n2c5ccccc5c5cccc(c52)B4c2cccc4c5ccccc5n-3c24)c2ccccc21]

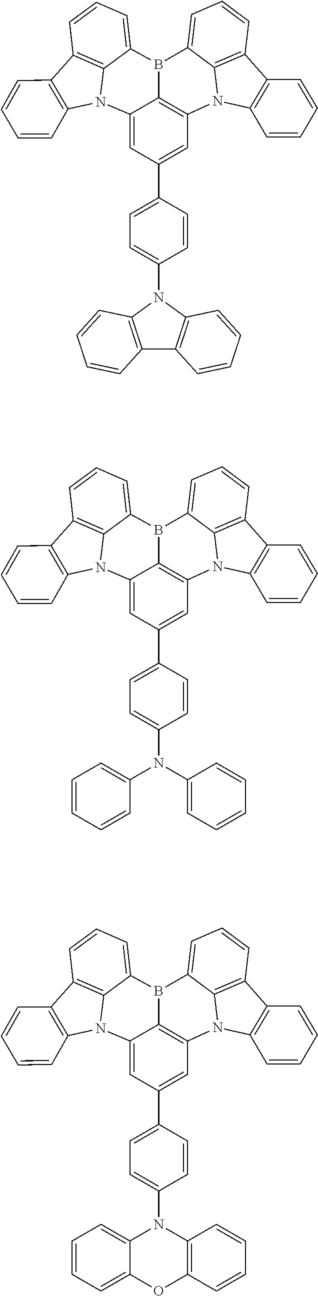 [SMILES: c1ccc2c(c1)c1ccccc1n2-c1ccc(-c2cc3c4c(c2)-n2c5ccccc5c5cccc(c52)B4c2cccc4c5ccccc5n-3c24)cc1 ; c1ccc(N(c2ccccc2)c2ccc(-c3cc4c5c(c3)-n3c6ccccc6c6cccc(c63)B5c3cccc5c6ccccc6n-4c35)cc2)cc1 ; c1ccc2c(c1)Oc1ccccc1N2c1ccc(-c2cc3c4c(c2)-n2c5ccccc5c5cccc(c52)B4c2cccc4c5ccccc5n-3c24)cc1]

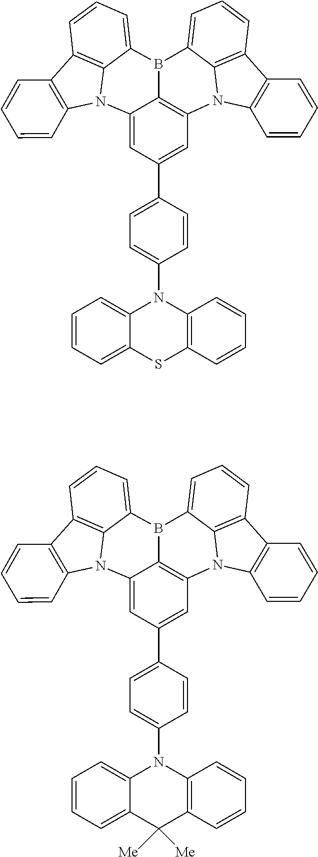 [SMILES: c1ccc2c(c1)Sc1ccccc1N2c1ccc(-c2cc3c4c(c2)-n2c5ccccc5c5cccc(c52)B4c2cccc4c5ccccc5n-3c24)cc1 ; CC1(C)c2ccccc2N(c2ccc(-c3cc4c5c(c3)-n3c6ccccc6c6cccc(c63)B5c3cccc5c6ccccc6n-4c35)cc2)c2ccccc21]

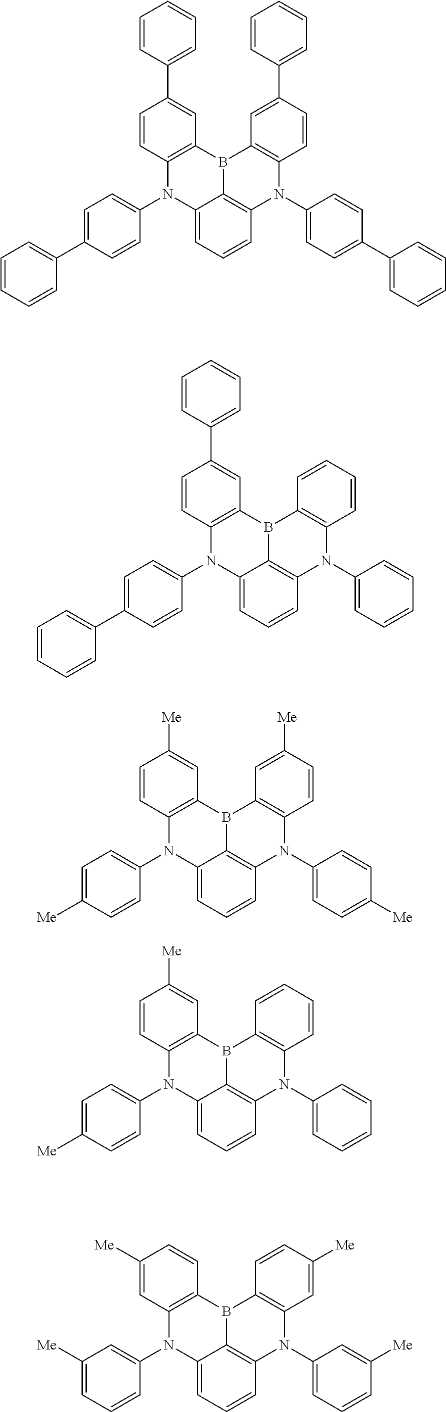 [SMILES: c1ccc(-c2ccc(N3c4ccc(-c5ccccc5)cc4B4c5cc(-c6ccccc6)ccc5N(c5ccc(-c6ccccc6)cc5)c5cccc3c54)cc2)cc1 ; c1ccc(-c2ccc(N3c4ccc(-c5ccccc5)cc4B4c5ccccc5N(c5ccccc5)c5cccc3c54)cc2)cc1 ; Cc1ccc(N2c3ccc(C)cc3B3c4cc(C)ccc4N(c4ccc(C)cc4)c4cccc2c43)cc1 ; Cc1ccc(N2c3ccc(C)cc3B3c4ccccc4N(c4ccccc4)c4cccc2c43)cc1 ; Cc1cccc(N2c3cc(C)ccc3B3c4ccc(C)cc4N(c4cccc(C)c4)c4cccc2c43)c1]

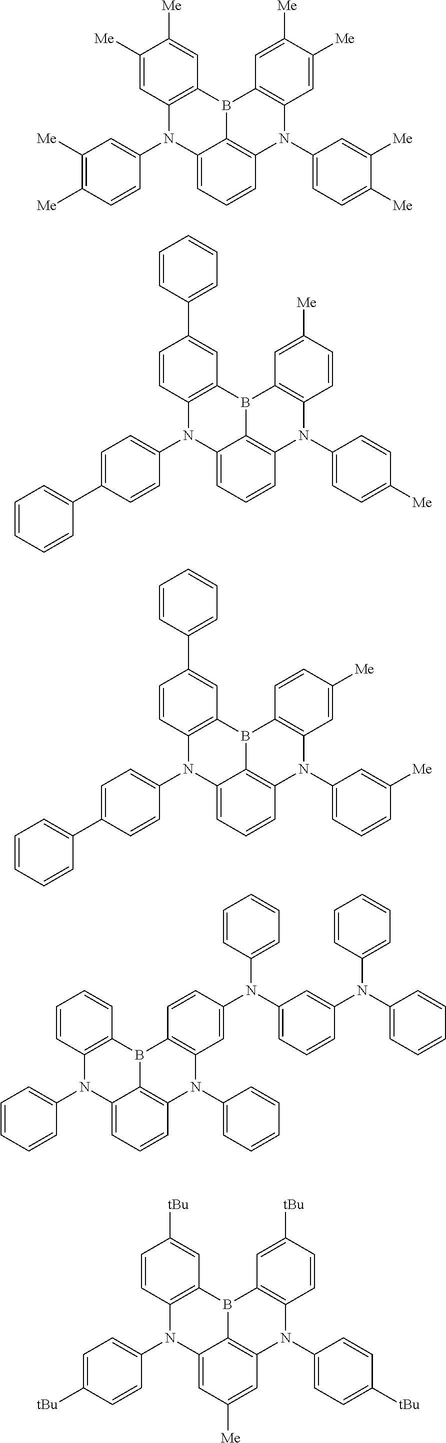 [SMILES: Cc1ccc(N2c3cc(C)c(C)cc3B3c4cc(C)c(C)cc4N(c4ccc(C)c(C)c4)c4cccc2c43)cc1C ; Cc1ccc(N2c3ccc(C)cc3B3c4cc(-c5ccccc5)ccc4N(c4ccc(-c5ccccc5)cc4)c4cccc2c43)cc1 ; Cc1cccc(N2c3cc(C)ccc3B3c4cc(-c5ccccc5)ccc4N(c4ccc(-c5ccccc5)cc4)c4cccc2c43)c1 ; c1ccc(N(c2ccccc2)c2cccc(N(c3ccccc3)c3ccc4c(c3)N(c3ccccc3)c3cccc5c3B4c3ccccc3N5c3ccccc3)c2)cc1 ; Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1]

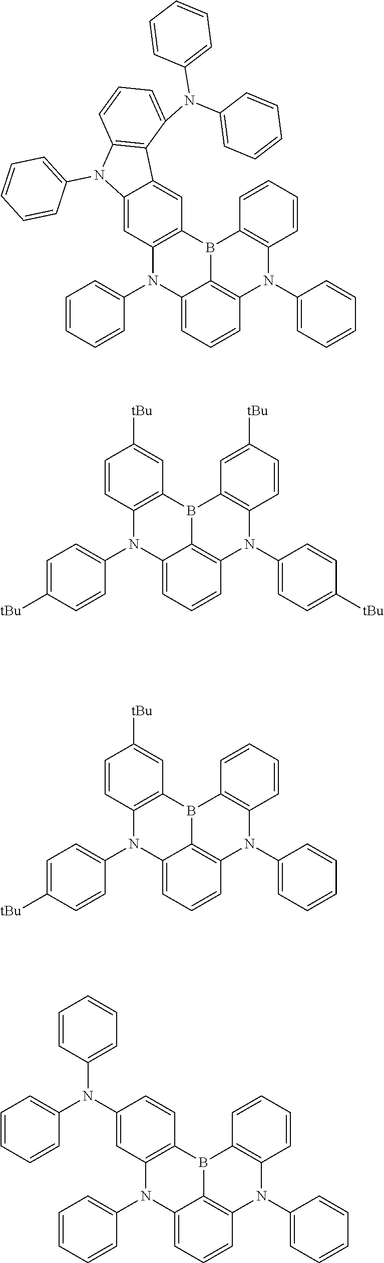 [SMILES: c1ccc(N2c3ccccc3B3c4cc5c6c(N(c7ccccc7)c7ccccc7)cccc6n(-c6ccccc6)c5cc4N(c4ccccc4)c4cccc2c43)cc1 ; CC(C)(C)c1ccc(N2c3ccc(C(C)(C)C)cc3B3c4cc(C(C)(C)C)ccc4N(c4ccc(C(C)(C)C)cc4)c4cccc2c43)cc1 ; CC(C)(C)c1ccc(N2c3ccc(C(C)(C)C)cc3B3c4ccccc4N(c4ccccc4)c4cccc2c43)cc1 ; c1ccc(N(c2ccccc2)c2ccc3c(c2)N(c2ccccc2)c2cccc4c2B3c2ccccc2N4c2ccccc2)cc1]

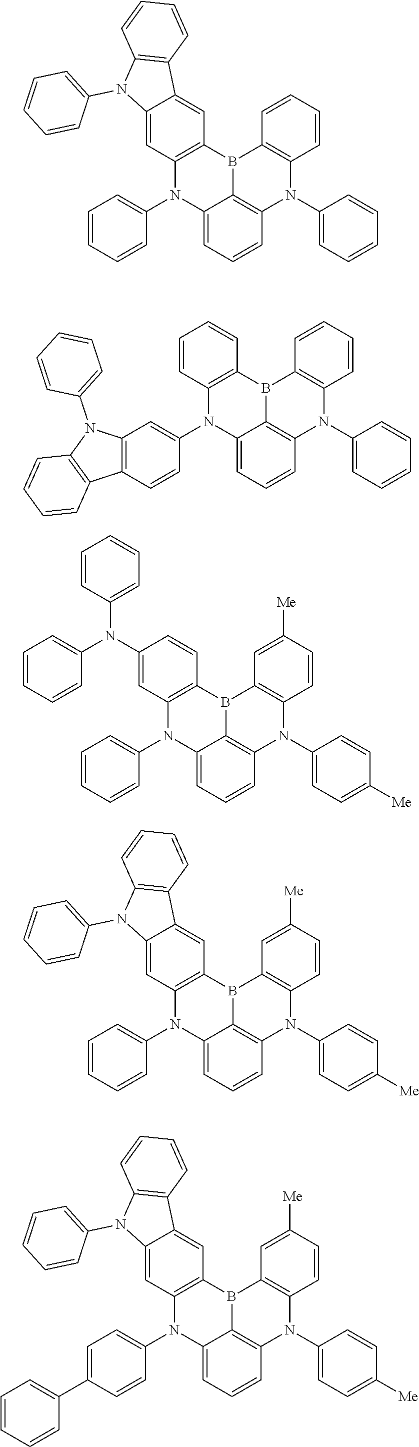

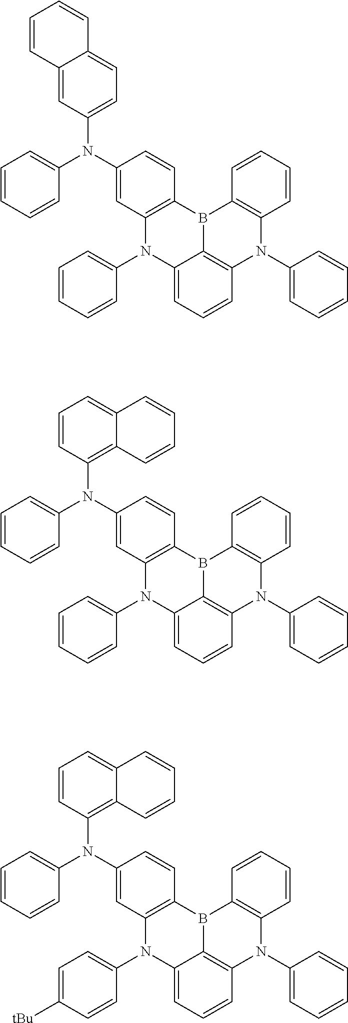 [SMILES: c1ccc(N(c2ccc3c(c2)N(c2ccccc2)c2cccc4c2B3c2ccccc2N4c2ccccc2)c2ccc3ccccc3c2)cc1 ; c1ccc(N2c3ccccc3B3c4ccc(N(c5ccccc5)c5cccc6ccccc56)cc4N(c4ccccc4)c4cccc2c43)cc1 ; CC(C)(C)c1ccc(N2c3cc(N(c4ccccc4)c4cccc5ccccc45)ccc3B3c4ccccc4N(c4ccccc4)c4cccc2c43)cc1]

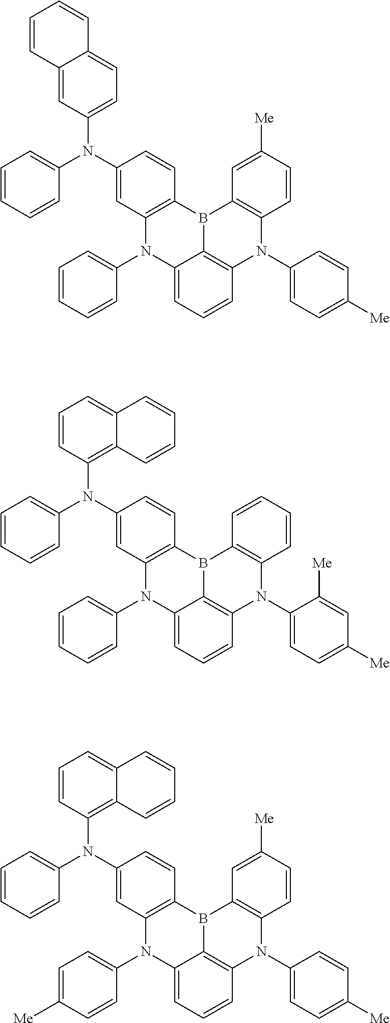 [SMILES: Cc1ccc(N2c3ccc(C)cc3B3c4ccc(N(c5ccccc5)c5ccc6ccccc6c5)cc4N(c4ccccc4)c4cccc2c43)cc1 ; Cc1ccc(N2c3ccccc3B3c4ccc(N(c5ccccc5)c5cccc6ccccc56)cc4N(c4ccccc4)c4cccc2c43)c(C)c1 ; Cc1ccc(N2c3ccc(C)cc3B3c4ccc(N(c5ccccc5)c5cccc6ccccc56)cc4N(c4ccc(C)cc4)c4cccc2c43)cc1]

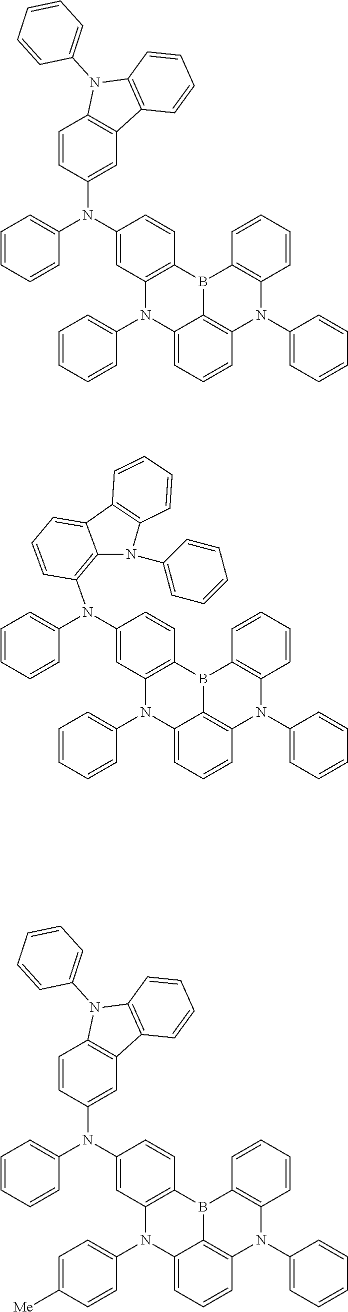 [SMILES: c1ccc(N(c2ccc3c(c2)N(c2ccccc2)c2cccc4c2B3c2ccccc2N4c2ccccc2)c2ccc3c(c2)c2ccccc2n3-c2ccccc2)cc1 ; c1ccc(N2c3ccccc3B3c4ccc(N(c5ccccc5)c5cccc6c7ccccc7n(-c7ccccc7)c56)cc4N(c4ccccc4)c4cccc2c43)cc1 ; Cc1ccc(N2c3cc(N(c4ccccc4)c4ccc5c(c4)c4ccccc4n5-c4ccccc4)ccc3B3c4ccccc4N(c4ccccc4)c4cccc2c43)cc1]

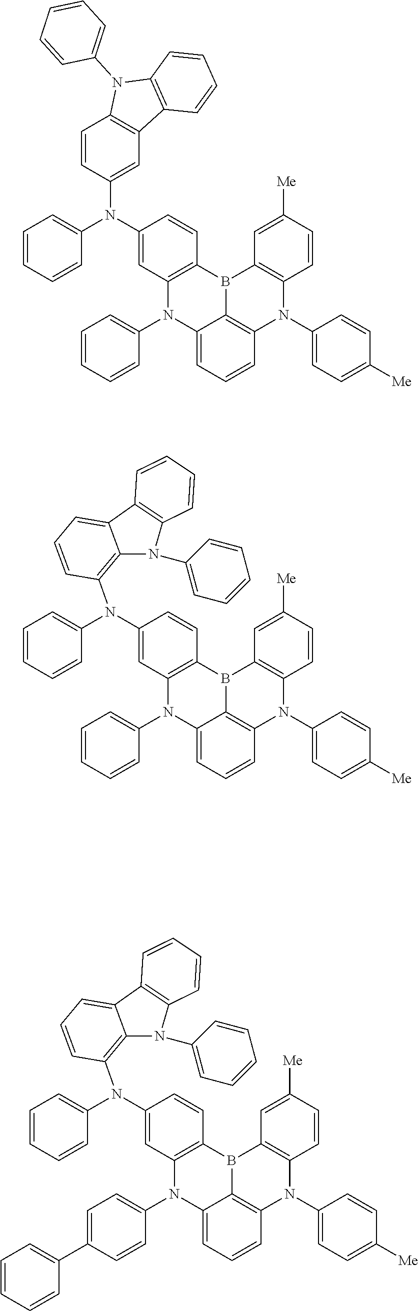 [SMILES: Cc1ccc(N2c3ccc(C)cc3B3c4ccc(N(c5ccccc5)c5ccc6c(c5)c5ccccc5n6-c5ccccc5)cc4N(c4ccccc4)c4cccc2c43)cc1 ; Cc1ccc(N2c3ccc(C)cc3B3c4ccc(N(c5ccccc5)c5cccc6c7ccccc7n(-c7ccccc7)c56)cc4N(c4ccccc4)c4cccc2c43)cc1 ; Cc1ccc(N2c3ccc(C)cc3B3c4ccc(N(c5ccccc5)c5cccc6c7ccccc7n(-c7ccccc7)c56)cc4N(c4ccc(-c5ccccc5)cc4)c4cccc2c43)cc1]

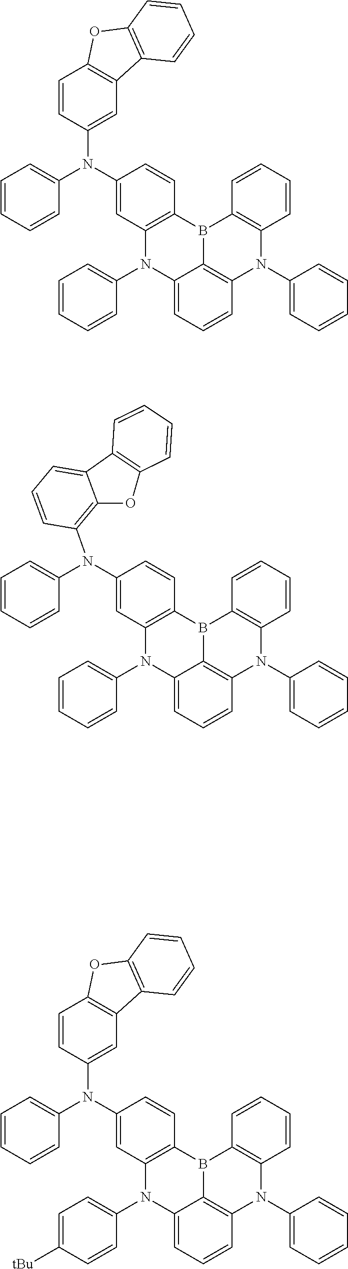 [SMILES: c1ccc(N(c2ccc3c(c2)N(c2ccccc2)c2cccc4c2B3c2ccccc2N4c2ccccc2)c2ccc3oc4ccccc4c3c2)cc1 ; c1ccc(N2c3ccccc3B3c4ccc(N(c5ccccc5)c5cccc6c5oc5ccccc56)cc4N(c4ccccc4)c4cccc2c43)cc1 ; CC(C)(C)c1ccc(N2c3cc(N(c4ccccc4)c4ccc5oc6ccccc6c5c4)ccc3B3c4ccccc4N(c4ccccc4)c4cccc2c43)cc1]

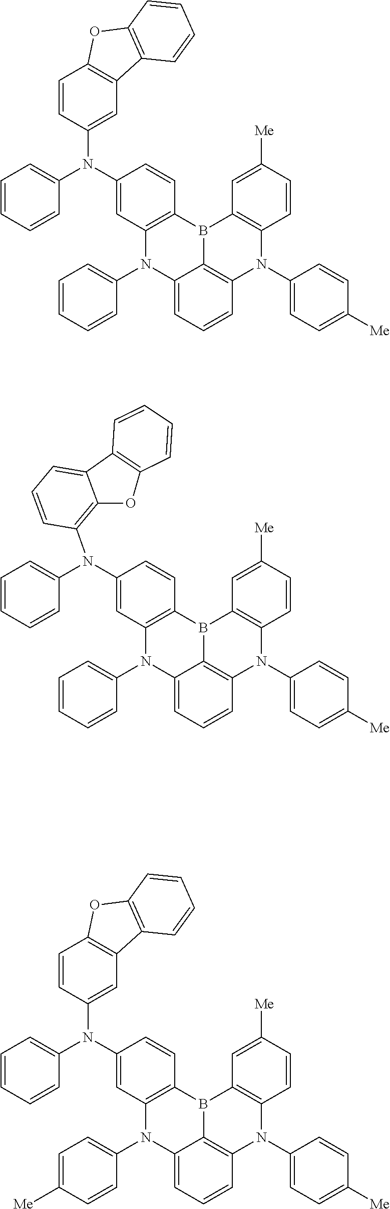 [SMILES: Cc1ccc(N2c3ccc(C)cc3B3c4ccc(N(c5ccccc5)c5ccc6oc7ccccc7c6c5)cc4N(c4ccccc4)c4cccc2c43)cc1 ; Cc1ccc(N2c3ccc(C)cc3B3c4ccc(N(c5ccccc5)c5cccc6c5oc5ccccc56)cc4N(c4ccccc4)c4cccc2c43)cc1 ; Cc1ccc(N2c3ccc(C)cc3B3c4ccc(N(c5ccccc5)c5ccc6oc7ccccc7c6c5)cc4N(c4ccc(C)cc4)c4cccc2c43)cc1]

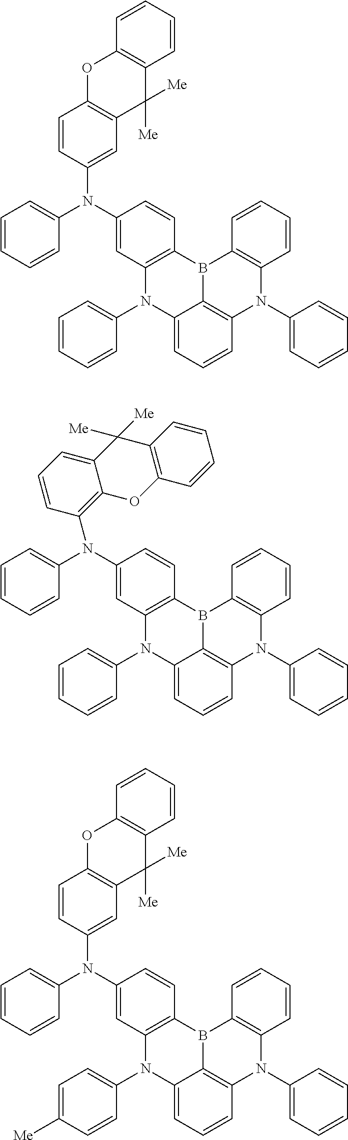 [SMILES: CC1(C)c2ccccc2Oc2ccc(N(c3ccccc3)c3ccc4c(c3)N(c3ccccc3)c3cccc5c3B4c3ccccc3N5c3ccccc3)cc21 ; CC1(C)c2ccccc2Oc2c(N(c3ccccc3)c3ccc4c(c3)N(c3ccccc3)c3cccc5c3B4c3ccccc3N5c3ccccc3)cccc21 ; Cc1ccc(N2c3cc(N(c4ccccc4)c4ccc5c(c4)C(C)(C)c4ccccc4O5)ccc3B3c4ccccc4N(c4ccccc4)c4cccc2c43)cc1]

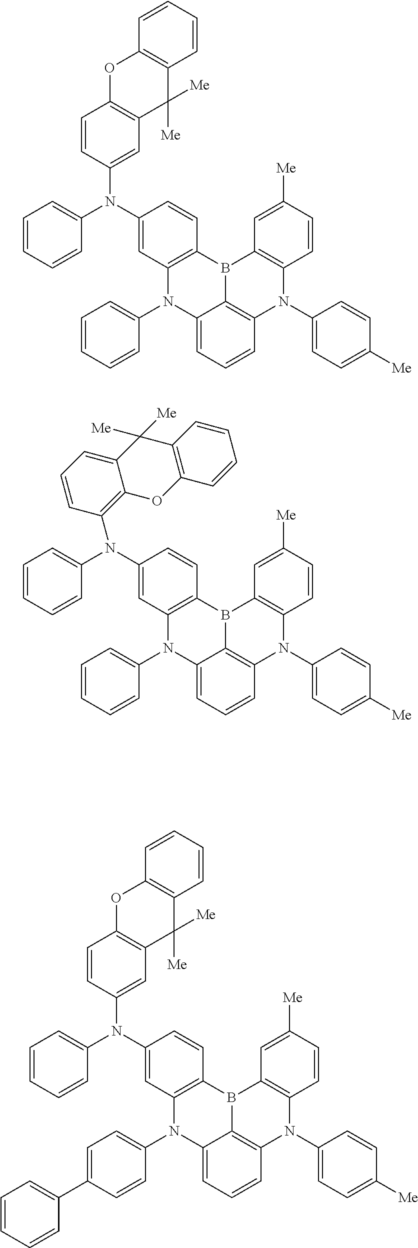 [SMILES: Cc1ccc(N2c3ccc(C)cc3B3c4ccc(N(c5ccccc5)c5ccc6c(c5)C(C)(C)c5ccccc5O6)cc4N(c4ccccc4)c4cccc2c43)cc1 ; Cc1ccc(N2c3ccc(C)cc3B3c4ccc(N(c5ccccc5)c5cccc6c5Oc5ccccc5C6(C)C)cc4N(c4ccccc4)c4cccc2c43)cc1 ; Cc1ccc(N2c3ccc(C)cc3B3c4ccc(N(c5ccccc5)c5ccc6c(c5)C(C)(C)c5ccccc5O6)cc4N(c4ccc(-c5ccccc5)cc4)c4cccc2c43)cc1]

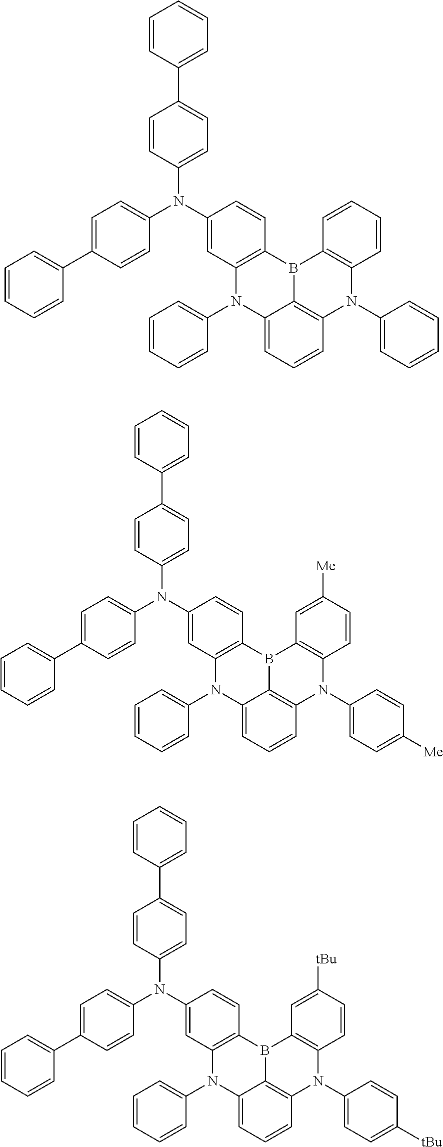 [SMILES: c1ccc(-c2ccc(N(c3ccc(-c4ccccc4)cc3)c3ccc4c(c3)N(c3ccccc3)c3cccc5c3B4c3ccccc3N5c3ccccc3)cc2)cc1 ; Cc1ccc(N2c3ccc(C)cc3B3c4ccc(N(c5ccc(-c6ccccc6)cc5)c5ccc(-c6ccccc6)cc5)cc4N(c4ccccc4)c4cccc2c43)cc1 ; CC(C)(C)c1ccc(N2c3ccc(C(C)(C)C)cc3B3c4ccc(N(c5ccc(-c6ccccc6)cc5)c5ccc(-c6ccccc6)cc5)cc4N(c4ccccc4)c4cccc2c43)cc1]

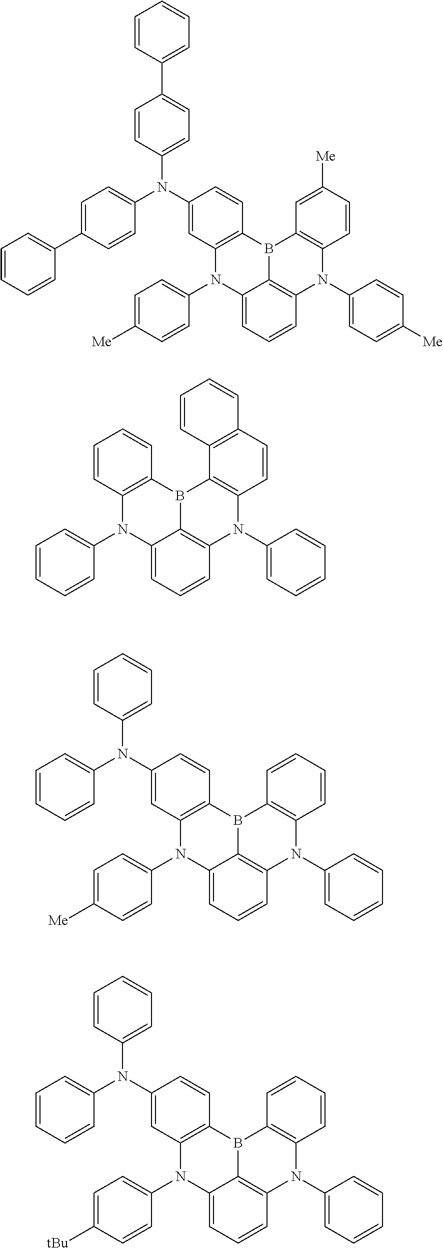 [SMILES: Cc1ccc(N2c3ccc(C)cc3B3c4ccc(N(c5ccc(-c6ccccc6)cc5)c5ccc(-c6ccccc6)cc5)cc4N(c4ccc(C)cc4)c4cccc2c43)cc1 ; c1ccc(N2c3ccccc3B3c4c2cccc4N(c2ccccc2)c2ccc4ccccc4c23)cc1 ; Cc1ccc(N2c3cc(N(c4ccccc4)c4ccccc4)ccc3B3c4ccccc4N(c4ccccc4)c4cccc2c43)cc1 ; CC(C)(C)c1ccc(N2c3cc(N(c4ccccc4)c4ccccc4)ccc3B3c4ccccc4N(c4ccccc4)c4cccc2c43)cc1]

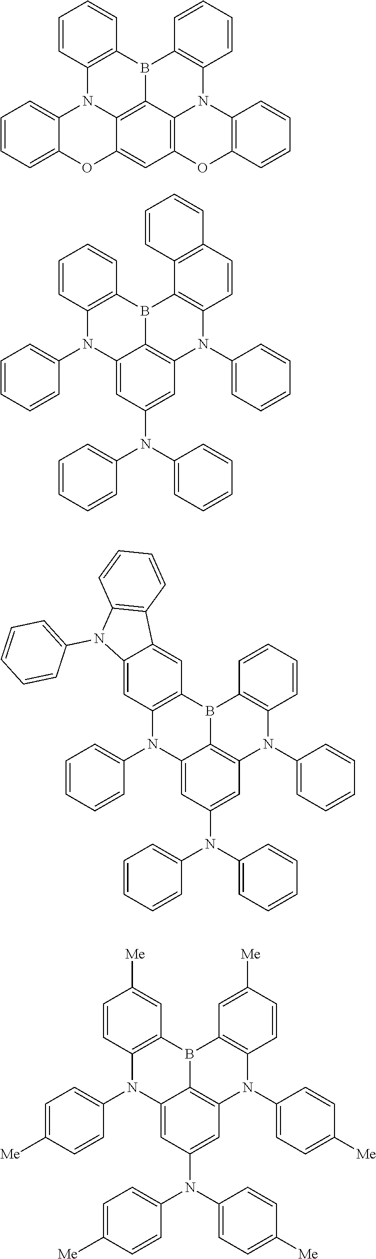

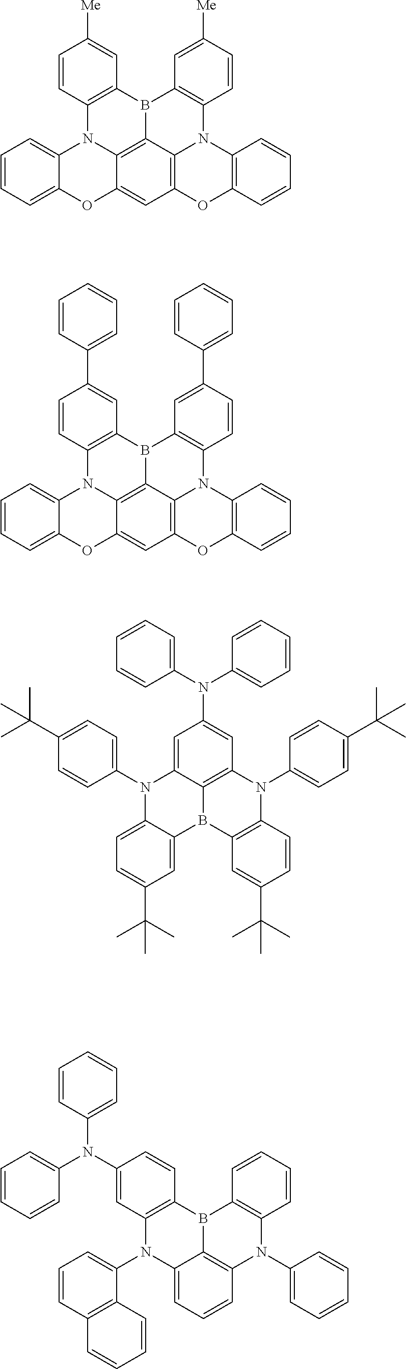 [SMILES: Cc1ccc2c(c1)B1c3cc(C)ccc3-n3c4ccccc4oc4cc5oc6ccccc6n-2c5c1c43 ; c1ccc(-c2ccc3c(c2)B2c4cc(-c5ccccc5)ccc4-n4c5ccccc5oc5cc6oc7ccccc7n-3c6c2c54)cc1 ; CC(C)(C)c1ccc(N2c3ccc(C(C)(C)C)cc3B3c4cc(C(C)(C)C)ccc4N(c4ccc(C(C)(C)C)cc4)c4cc(N(c5ccccc5)c5ccccc5)cc2c43)cc1 ; c1ccc(N(c2ccccc2)c2ccc3c(c2)N(c2cccc4ccccc24)c2cccc4c2B3c2ccccc2N4c2ccccc2)cc1]

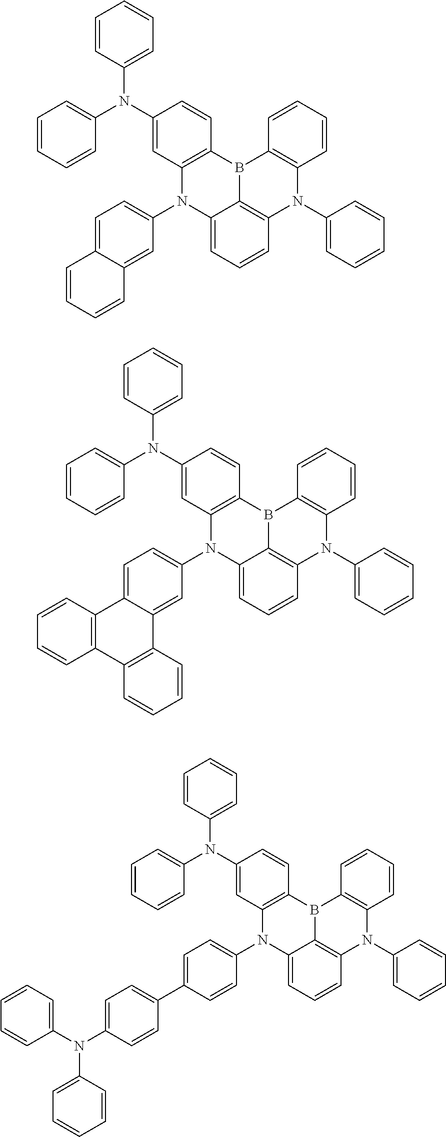 [SMILES: c1ccc(N(c2ccccc2)c2ccc3c(c2)N(c2ccc4ccccc4c2)c2cccc4c2B3c2ccccc2N4c2ccccc2)cc1 ; c1ccc(N(c2ccccc2)c2ccc3c(c2)N(c2ccc4c5ccccc5c5ccccc5c4c2)c2cccc4c2B3c2ccccc2N4c2ccccc2)cc1 ; c1ccc(N(c2ccccc2)c2ccc(-c3ccc(N4c5cc(N(c6ccccc6)c6ccccc6)ccc5B5c6ccccc6N(c6ccccc6)c6cccc4c65)cc3)cc2)cc1]

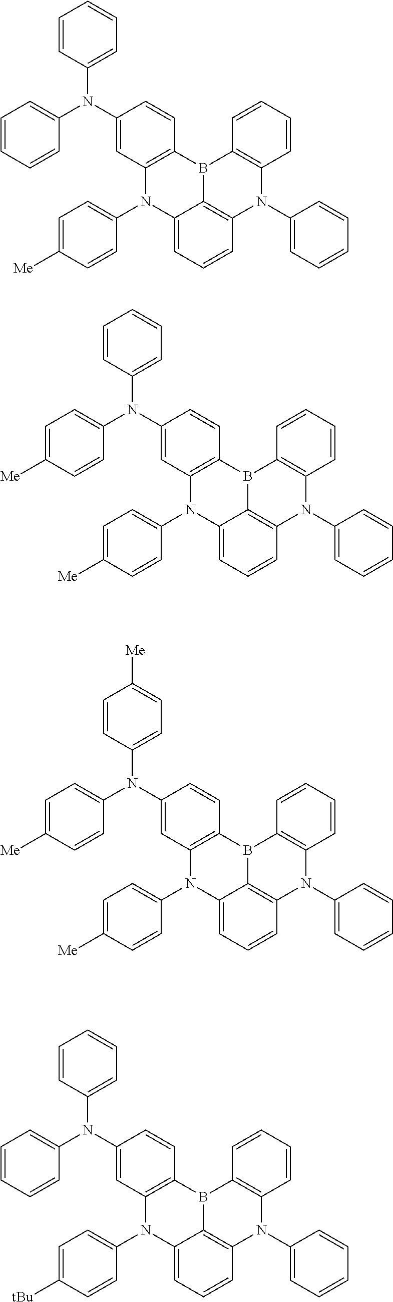 [SMILES: Cc1ccc(N2c3cc(N(c4ccccc4)c4ccccc4)ccc3B3c4ccccc4N(c4ccccc4)c4cccc2c43)cc1 ; Cc1ccc(N(c2ccccc2)c2ccc3c(c2)N(c2ccc(C)cc2)c2cccc4c2B3c2ccccc2N4c2ccccc2)cc1 ; Cc1ccc(N(c2ccc(C)cc2)c2ccc3c(c2)N(c2ccc(C)cc2)c2cccc4c2B3c2ccccc2N4c2ccccc2)cc1 ; CC(C)(C)c1ccc(N2c3cc(N(c4ccccc4)c4ccccc4)ccc3B3c4ccccc4N(c4ccccc4)c4cccc2c43)cc1]

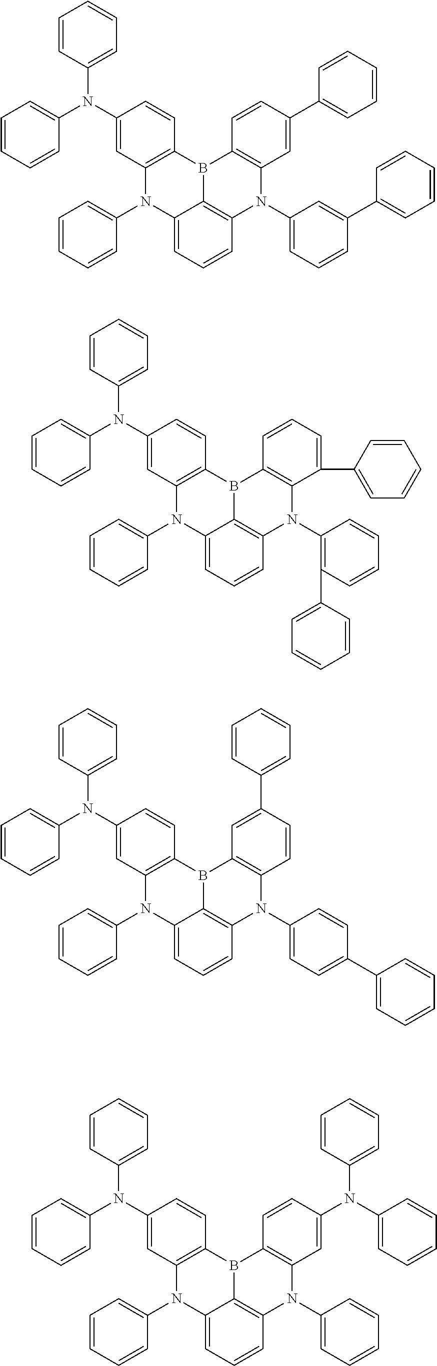 [SMILES: c1ccc(-c2cccc(N3c4cc(-c5ccccc5)ccc4B4c5ccc(N(c6ccccc6)c6ccccc6)cc5N(c5ccccc5)c5cccc3c54)c2)cc1 ; c1ccc(-c2ccccc2N2c3cccc4c3B(c3ccc(N(c5ccccc5)c5ccccc5)cc3N4c3ccccc3)c3cccc(-c4ccccc4)c32)cc1 ; c1ccc(-c2ccc(N3c4ccc(-c5ccccc5)cc4B4c5ccc(N(c6ccccc6)c6ccccc6)cc5N(c5ccccc5)c5cccc3c54)cc2)cc1 ; c1ccc(N(c2ccccc2)c2ccc3c(c2)N(c2ccccc2)c2cccc4c2B3c2ccc(N(c3ccccc3)c3ccccc3)cc2N4c2ccccc2)cc1]

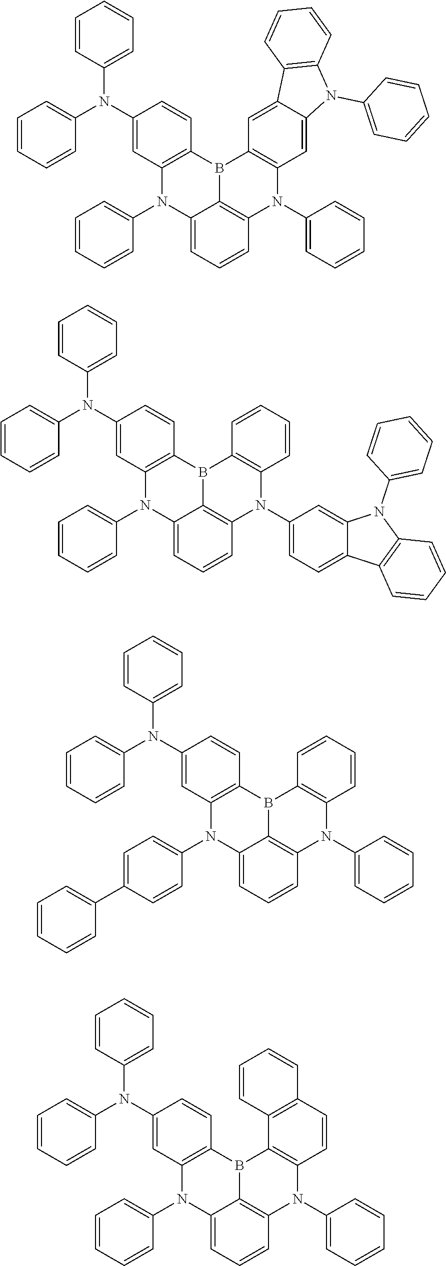

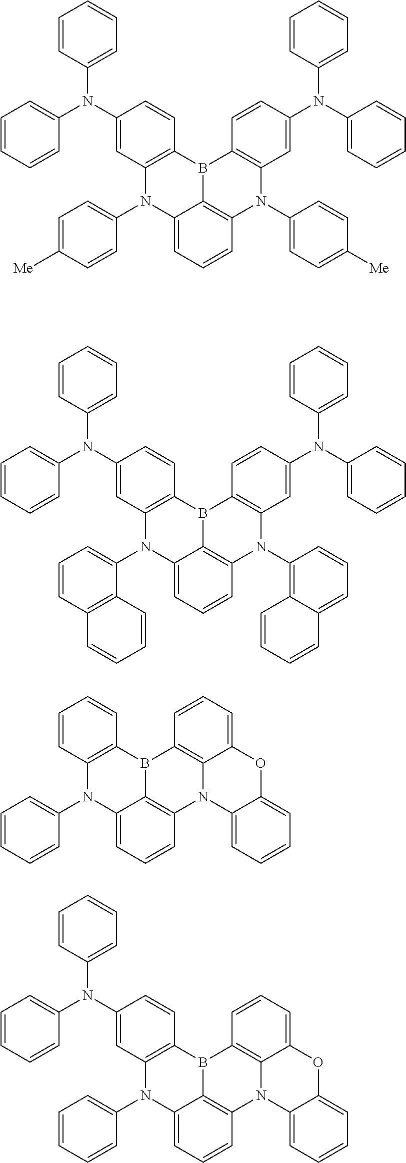

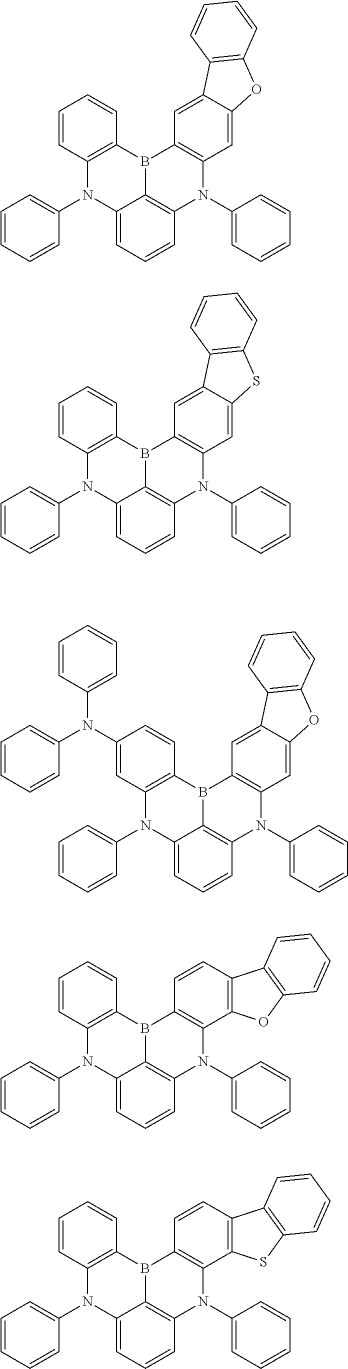

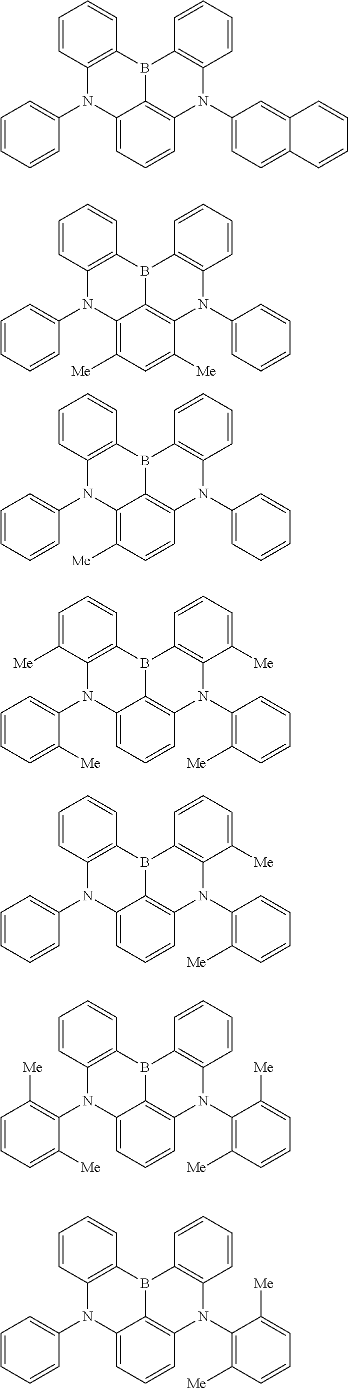

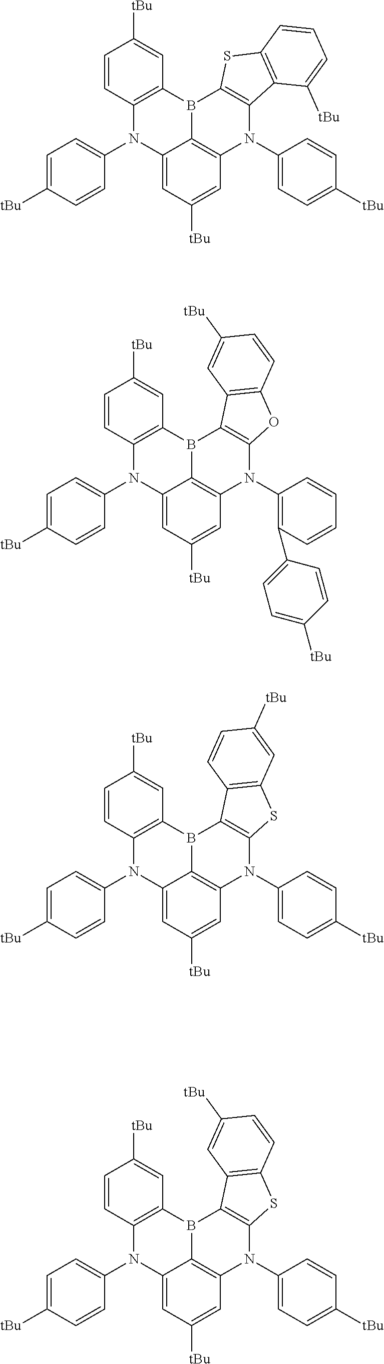 [SMILES: CC(C)(C)c1ccc(N2c3ccc(C(C)(C)C)cc3B3c4sc5cccc(C(C)(C)C)c5c4N(c4ccc(C(C)(C)C)cc4)c4cc(C(C)(C)C)cc2c43)cc1 ; CC(C)(C)c1ccc(-c2ccccc2N2c3cc(C(C)(C)C)cc4c3B(c3cc(C(C)(C)C)ccc3N4c3ccc(C(C)(C)C)cc3)c3c2oc2ccc(C(C)(C)C)cc32)cc1 ; CC(C)(C)c1ccc(N2c3ccc(C(C)(C)C)cc3B3c4c2cc(C(C)(C)C)cc4N(c2ccc(C(C)(C)C)cc2)c2sc4cc(C(C)(C)C)ccc4c23)cc1 ; CC(C)(C)c1ccc(N2c3ccc(C(C)(C)C)cc3B3c4c2cc(C(C)(C)C)cc4N(c2ccc(C(C)(C)C)cc2)c2sc4ccc(C(C)(C)C)cc4c23)cc1]

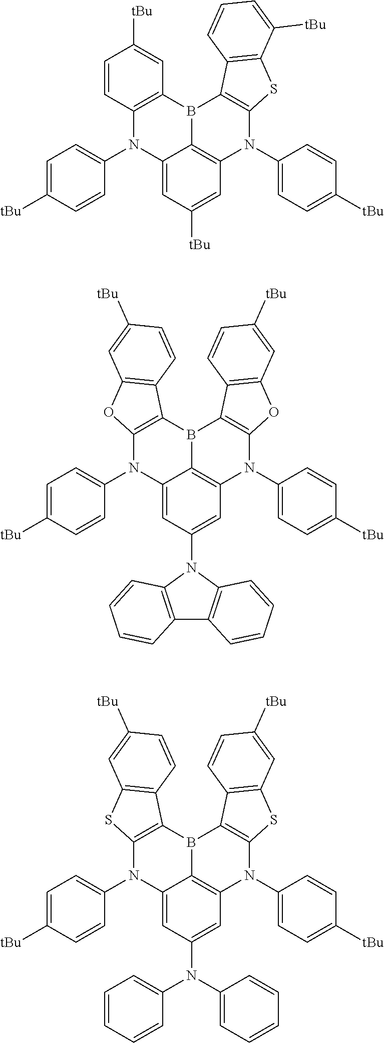 [SMILES: CC(C)(C)c1ccc(N2c3ccc(C(C)(C)C)cc3B3c4c2cc(C(C)(C)C)cc4N(c2ccc(C(C)(C)C)cc2)c2sc4c(C(C)(C)C)cccc4c23)cc1 ; CC(C)(C)c1ccc(N2c3cc(-n4c5ccccc5c5ccccc54)cc4c3B(c3c2oc2cc(C(C)(C)C)ccc32)c2c(oc3cc(C(C)(C)C)ccc23)N4c2ccc(C(C)(C)C)cc2)cc1 ; CC(C)(C)c1ccc(N2c3cc(N(c4ccccc4)c4ccccc4)cc4c3B(c3c2sc2cc(C(C)(C)C)ccc32)c2c(sc3cc(C(C)(C)C)ccc23)N4c2ccc(C(C)(C)C)cc2)cc1]

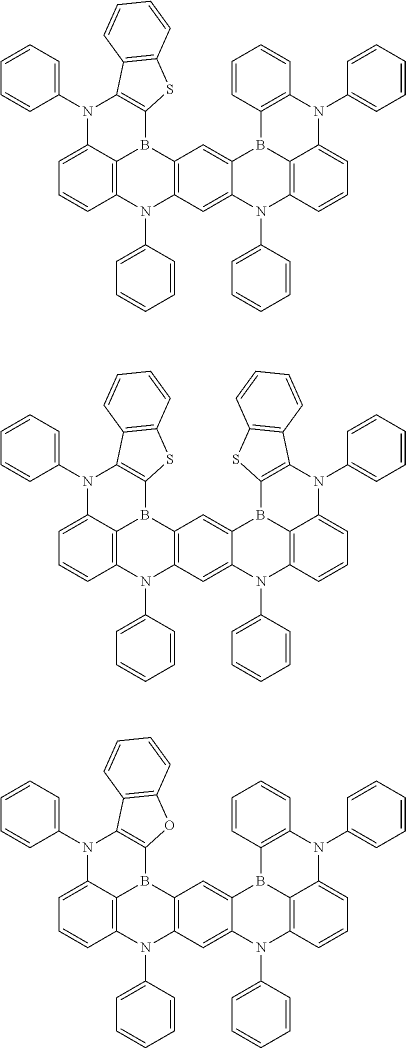 [SMILES: c1ccc(N2c3ccccc3B3c4cc5c(cc4N(c4ccccc4)c4cccc2c43)N(c2ccccc2)c2cccc3c2B5c2sc4ccccc4c2N3c2ccccc2)cc1 ; c1ccc(N2c3cc4c(cc3B3c5sc6ccccc6c5N(c5ccccc5)c5cccc2c53)B2c3sc5ccccc5c3N(c3ccccc3)c3cccc(c32)N4c2ccccc2)cc1 ; c1ccc(N2c3ccccc3B3c4cc5c(cc4N(c4ccccc4)c4cccc2c43)N(c2ccccc2)c2cccc3c2B5c2oc4ccccc4c2N3c2ccccc2)cc1]

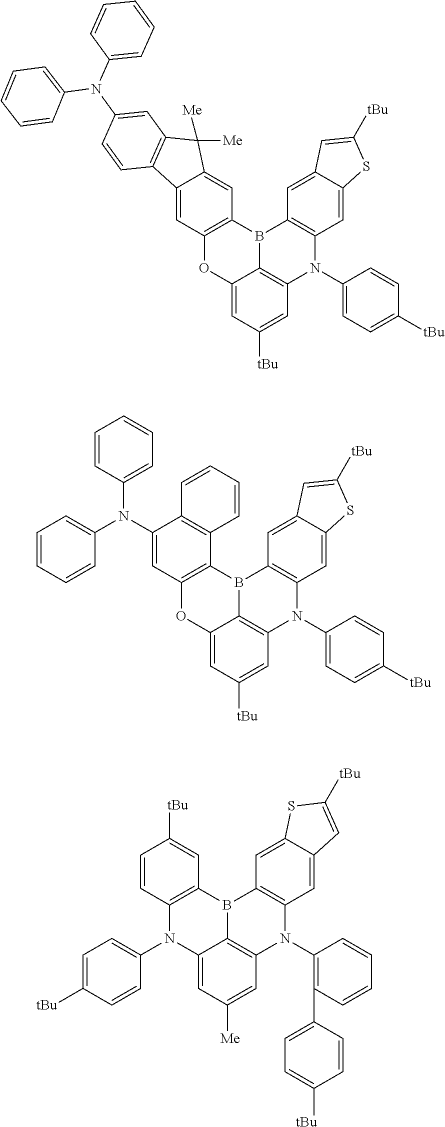 [SMILES: CC(C)(C)c1ccc(N2c3cc4sc(C(C)(C)C)cc4cc3B3c4cc5c(cc4Oc4cc(C(C)(C)C)cc2c43)-c2ccc(N(c3ccccc3)c3ccccc3)cc2C5(C)C)cc1 ; CC(C)(C)c1ccc(N2c3cc4sc(C(C)(C)C)cc4cc3B3c4c(cc(C(C)(C)C)cc42)Oc2cc(N(c4ccccc4)c4ccccc4)c4ccccc4c23)cc1 ; Cc1cc2c3c(c1)N(c1ccccc1-c1ccc(C(C)(C)C)cc1)c1cc4cc(C(C)(C)C)sc4cc1B3c1cc(C(C)(C)C)ccc1N2c1ccc(C(C)(C)C)cc1]

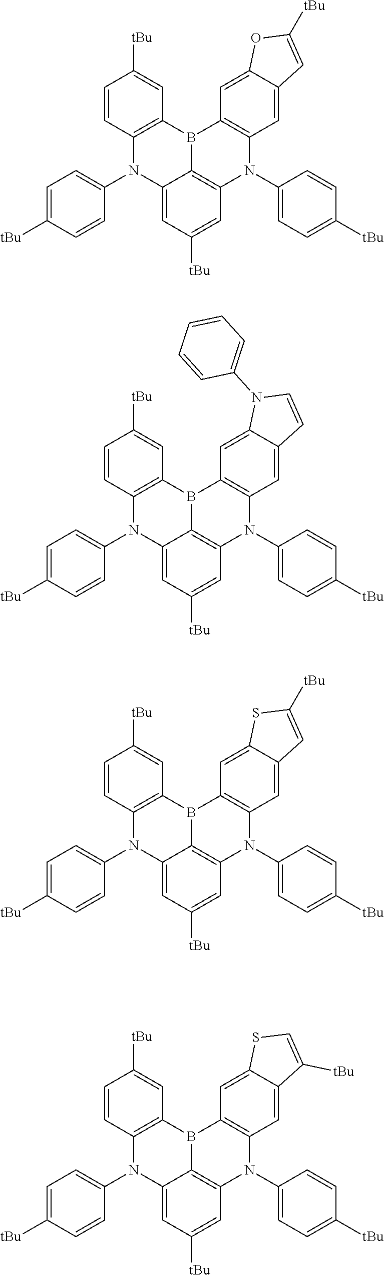 [SMILES: CC(C)(C)c1ccc(N2c3ccc(C(C)(C)C)cc3B3c4cc5oc(C(C)(C)C)cc5cc4N(c4ccc(C(C)(C)C)cc4)c4cc(C(C)(C)C)cc2c43)cc1 ; CC(C)(C)c1ccc(N2c3ccc(C(C)(C)C)cc3B3c4cc5c(ccn5-c5ccccc5)cc4N(c4ccc(C(C)(C)C)cc4)c4cc(C(C)(C)C)cc2c43)cc1 ; CC(C)(C)c1ccc(N2c3ccc(C(C)(C)C)cc3B3c4cc5sc(C(C)(C)C)cc5cc4N(c4ccc(C(C)(C)C)cc4)c4cc(C(C)(C)C)cc2c43)cc1 ; CC(C)(C)c1ccc(N2c3ccc(C(C)(C)C)cc3B3c4cc5scc(C(C)(C)C)c5cc4N(c4ccc(C(C)(C)C)cc4)c4cc(C(C)(C)C)cc2c43)cc1]

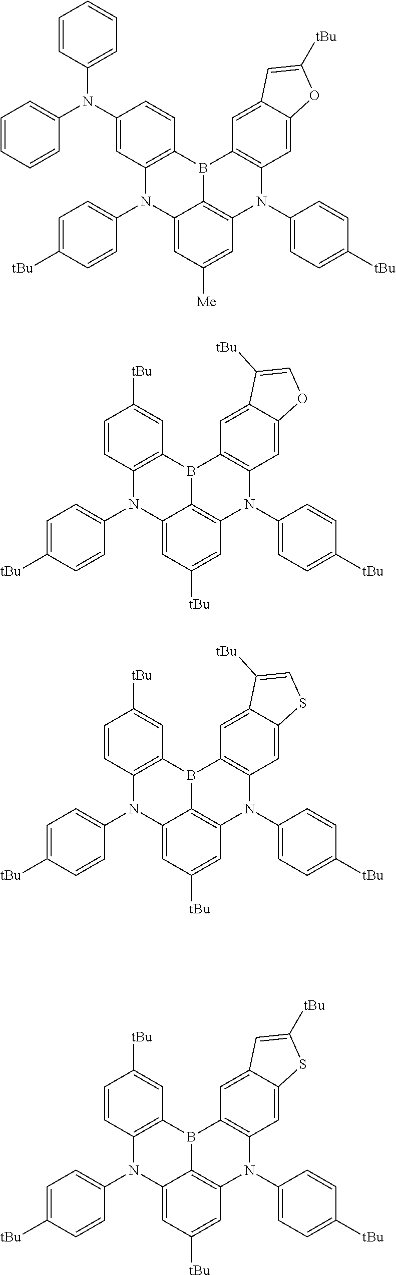 [SMILES: Cc1cc2c3c(c1)N(c1ccc(C(C)(C)C)cc1)c1cc4oc(C(C)(C)C)cc4cc1B3c1ccc(N(c3ccccc3)c3ccccc3)cc1N2c1ccc(C(C)(C)C)cc1 ; CC(C)(C)c1ccc(N2c3ccc(C(C)(C)C)cc3B3c4cc5c(C(C)(C)C)coc5cc4N(c4ccc(C(C)(C)C)cc4)c4cc(C(C)(C)C)cc2c43)cc1 ; CC(C)(C)c1ccc(N2c3ccc(C(C)(C)C)cc3B3c4cc5c(C(C)(C)C)csc5cc4N(c4ccc(C(C)(C)C)cc4)c4cc(C(C)(C)C)cc2c43)cc1 ; CC(C)(C)c1ccc(N2c3ccc(C(C)(C)C)cc3B3c4cc5cc(C(C)(C)C)sc5cc4N(c4ccc(C(C)(C)C)cc4)c4cc(C(C)(C)C)cc2c43)cc1]

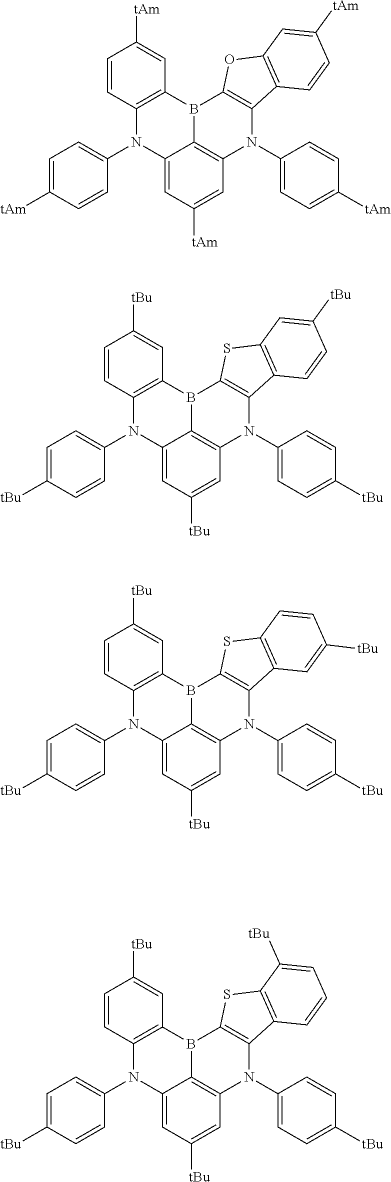

In the emitting compounds such as the first emitting compound and the second emitting compound, R₉₀₁, R₉₀₂, R₉₀₃, R₉₀₄, R₉₀₅, R₉₀₆, and R₉₀₇ are each independently a hydrogen atom, a substituted or unsubstituted alkyl group having to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms, preferably a substituted or unsubstituted alkyl group having 1 to 50 carbon atoms, or a substituted or unsubstituted aryl group having 6 to 50 ring carbon atoms;
when a plurality of R₉₀₂ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different; and
when a plurality of R₉₀₇ are present, the plurality of R₉₀₇ are mutually the same or different.

### Additional Layers of Organic EL Device

The organic EL device according to the third exemplary embodiment may include at least one layer formed from an organic compound in addition to the first emitting layer and the second emitting layer.

Examples of the layer formed from an organic compound include at least one layer selected from the group consisting of a hole injecting layer, a hole transporting layer, an electron blocking layer, a hole blocking layer, an electron injecting layer, and an electron transporting layer.

The layer formed from an organic compound may further contain an inorganic compound.

In the organic EL device according to the third exemplary embodiment, the organic layer may be composed only of the first emitting layer and the second emitting layer, but may further include, for instance, at least one layer selected from the group consisting of a hole injecting layer, a hole transporting layer, an electron blocking layer, a hole blocking layer, an electron injecting layer, and an electron transporting layer.

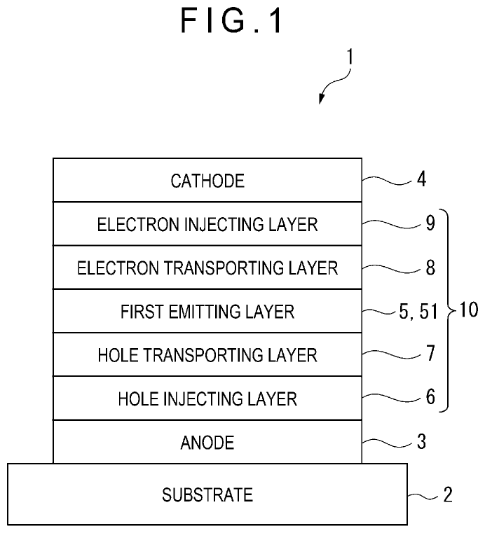

FIG. 1 schematically depicts an exemplary arrangement of an organic EL device according to the exemplary embodiment.

An organic EL device 1 includes a light-transmissive substrate 2, an anode 3, a cathode 4, and organic layers 10 provided between the anode 3 and the cathode 4.

The organic layers 10 include a hole injecting layer 6, a hole transporting layer 7, a first emitting layer 51, an electron transporting layer 8, and an electron injecting layer 9 that are layered in this order on the anode 3.

An emitting zone 5 of the organic EL device 1 includes only the first emitting layer 51.

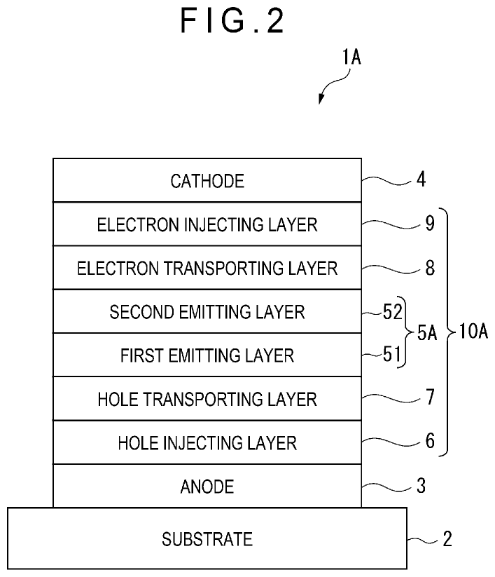

FIG. 2 schematically depicts an exemplary arrangement of an organic EL device according to the exemplary embodiment.

An organic EL device 1A includes a light-transmissive substrate 2, an anode 3, a cathode 4, and organic layers 10A provided between the anode 3 and the cathode 4.

The organic layers 10A include a hole injecting layer 6, a hole transporting layer 7, a first emitting layer 51, a second emitting layer 52, an electron transporting layer 8, and an electron injecting layer 9 that are layered in this order on the anode 3.

An emitting zone 5A of the organic EL device 1A includes the first emitting layer 51 on a side close to anode 3 and the second emitting layer 52 on a side close to the cathode 4.

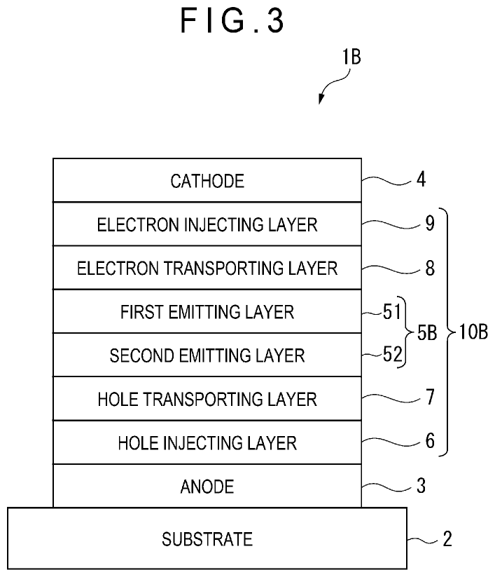

FIG. 3 schematically depicts still another exemplary arrangement of the organic EL device according to the third exemplary embodiment.

An organic EL device 1B includes a light-transmissive substrate 2, an anode 3, a cathode 4, and organic layers 10B provided between the anode 3 and the cathode 4.

The organic layers 10B include a hole injecting layer 6, a hole transporting layer 7, a second emitting layer 52, a first emitting layer 51, an electron transporting layer 8, and an electron injecting layer 9 that are layered in this order on the anode 3.

An emitting zone 5B of the organic EL device 1B includes the second emitting layer 52 on a side close to the anode 3 and the first emitting layer 51 on a side close to the cathode 4.

The invention is not limited to the arrangements of the organic EL device depicted in FIGS. 1, 2, and 3.

An arrangement of the organic EL device will be further described below.

It should be noted that the reference numerals are occasionally omitted below.

In the organic EL device according to the third exemplary embodiment, a layer formed from an organic compound may be provided between the first emitting layer and the second emitting layer.

### Interposed Layer

The organic EL device according to the third exemplary embodiment may include an interposed layer as the layer formed from an organic compound provided between the first emitting layer and the second emitting layer.

In the third exemplary embodiment, in order for a Singlet emitting region and a TTF emitting region not to overlap each other, the interposed layer contains no emitting compound or may contain an emitting compound in an insubstantial amount provided that the overlap can be inhibited.

For instance, the interposed layer contains 0 mass % of an emitting compound. Alternatively, for instance, the interposed layer may contain an emitting compound provided that the emitting compound contained is a component accidentally mixed in a manufacturing process or a component contained as impurities in a material.

For instance, when the interposed layer consists of a material A, a material B, and a material C, the content ratios of the materials A, B, and C in the interposed layer are each 10 mass % or more, and the total of the content ratios of the materials A, B, and C is 100 mass %.

Hereinafter, the interposed layer is occasionally referred to as a “non-doped layer”.

A layer containing an emitting compound is occasionally referred to as a “doped layer”.

It is considered that luminous efficiency is improvable in an arrangement including layered emitting layers because the Singlet emitting region and the TTF emitting region are typically likely to be separated from each other.

In the organic EL device according to the third exemplary embodiment, when the interposed layer (non-doped layer) is provided between the first emitting layer and the second emitting layer in the emitting zone, it is expected that the region where the Singlet emitting region and the TTF emitting region overlap each other will be reduced, and a decrease in TTF efficiency due to collision of triplet excitons with carriers will be inhibited.

That is, the interposition of the interposed layer (non-doped layer) between the emitting layers is considered to contribute to improving the efficiency of TTF emission.

The interposed layer is a non-doped layer.

The interposed layer contains no metal atom.

Therefore, the interposed layer contains no metal complex.

The interposed layer contains an interposed layer material.

The interposed layer material is not an emitting compound.

The interposed layer material may be any material except for the emitting compound.

Examples of the interposed layer material include 1) heterocyclic compounds such as oxadiazole derivatives, benzimidazole derivatives, and phenanthroline derivatives, 2) fused aromatic compounds such as carbazole derivatives, anthracene derivatives, phenanthrene derivatives, pyrene derivatives, and chrysene derivatives, and 3) aromatic amine compounds such as triarylamine derivatives and fused polycyclic aromatic amine derivatives.

One or both of the first host material and the second host material may be used as the interposed layer material. The interposed layer material may be any material provided that the Singlet emitting region and the TTF emitting region are separated from each other and the Singlet emission and the TTF emission are not hindered.

In the interposed layer of the organic EL device according to the third exemplary embodiment, the content ratios of all the materials constituting the interposed layer are each 10 mass % or more.

The interposed layer contains the interposed layer material as a material constituting the interposed layer.

The interposed layer contains the interposed layer material preferably at 60 mass % or more, more preferably at 70 mass % or more, still more preferably at 80 mass % or more, still further more preferably at 90 mass % or more, and yet still further more preferably at 95 mass % or more with respect to the total mass of the interposed layer.

The interposed layer may contain a single type of the interposed layer material or may contain two or more types of the interposed layer material.

When the interposed layer contains two or more types of the interposed layer material, the upper limit of the total of the content ratios of the two or more types of the interposed layer material is 100 mass %.

In the third exemplary embodiment, the interposed layer may further contain any other material than the interposed layer material.

The interposed layer may be provided in the form of a single layer or a laminate of two or more layers.

The thickness of the interposed layer is not particularly limited as long as the Singlet emitting region and the TTF emitting region can be inhibited from overlapping with each other, and the thickness per layer is preferably in a range from 3 nm to 15 nm, more preferably in a range from 5 nm to 10 nm.

When the thickness of the interposed layer is 3 nm or more, the Singlet emitting region and the emitting region derived from TTF are easily separated from each other.

When the thickness of the interposed layer is 15 nm or less, a phenomenon in which the host material of the interposed layer emits light is easily inhibited.

The interposed layer contains the interposed layer material as a material constituting the interposed layer, and the triplet energy T₁(H1) of the first host material, the triplet energy T₁(H2) of the second host material, and a triplet energy T₁(M_{mid}) of at least one interposed layer material preferably satisfy a relationship of a numerical formula (Numerical Formula 21) below.

T₁(H1)≥T₁(M_{mid})≥T₁(H2) (Numerical Formula 21)

When the interposed layer contains two or more interposed layer materials as materials constituting the interposed layer, the triplet energy T₁(H1) of the first host material, the triplet energy T₁(H2) of the second host material, and a triplet energy T₁(M_{EA}) of each interposed layer material more preferably satisfy a relationship of a numerical formula (Numerical Formula 21A) below.

T₁(H1)≥T₁(M_{EA})≥T₁(H2) (Numerical Formula 21A)

### Substrate

The substrate is used as a support for the organic EL device.

For instance, glass, quartz, plastics and the like are usable for the substrate.

A flexible substrate is also usable.

The flexible substrate is a bendable substrate, which is exemplified by a plastic substrate.

Examples of the material for the plastic substrate include polycarbonate, polyarylate, polyethersulfone, polypropylene, polyester, polyvinyl fluoride, polyvinyl chloride, polyimide, and polyethylene naphthalate.

Moreover, an inorganic vapor deposition film is also usable.

### Anode

Metal, an alloy, an electrically conductive compound, a mixture thereof, or the like having a large work function (specifically, 4.0 eV or more) is preferably used as the anode formed on the substrate.

Specific examples of the material include indium oxide-tin oxide (ITO: indium tin oxide), indium oxide-tin oxide containing silicon or silicon oxide, indium oxide-zinc oxide, indium oxide containing tungsten oxide and zinc oxide, and graphene.

In addition, gold (Au), platinum (Pt), nickel (Ni), tungsten (W), chrome (Cr), molybdenum (Mo), iron (Fe), cobalt (Co), copper (Cu), palladium (Pd), titanium (Ti), and nitrides of a metal material (e.g., titanium nitride) are usable.

The material is typically formed into a film by a sputtering method.

For instance, the indium oxide-zinc oxide can be formed into a film by the sputtering method using a target in which zinc oxide in a range from 1 mass % to 10 mass % is added to indium oxide.

Moreover, for instance, the indium oxide containing tungsten oxide and zinc oxide can be formed by the sputtering method using a target in which tungsten oxide in a range from 0.5 mass % to 5 mass % and zinc oxide in a range from 0.1 mass % to 1 mass % are added to indium oxide.

In addition, a vacuum deposition method, a coating method, an inkjet method, a spin coating method or the like may be usable.

Among the EL layers formed on the anode, the hole injecting layer adjacent to the anode is formed using a composite material into which holes are easily injectable irrespective of the work function of the anode, and thus materials usable as electrode materials (e.g., metals, alloys, electrically conductive compounds, mixtures thereof, and elements belonging to group 1 or 2 of the periodic table of the elements) are also usable for the anode.

A material having a small work function such as elements belonging to Groups 1 and 2 in the periodic table of the elements, specifically, an alkali metal such as lithium (Li) and cesium (Cs), an alkaline earth metal such as magnesium (Mg), calcium (Ca) and strontium (Sr), alloys (e.g., MgAg and AlLi) including the alkali metal or the alkaline earth metal, a rare earth metal such as europium (Eu) and ytterbium (Yb), alloys including the rare earth metal are also usable for the anode.

It should be noted that the vacuum deposition method and the sputtering method are usable for forming the anode using the alkali metal, alkaline earth metal and the alloy thereof.

Further, when a silver paste is used for the anode, the coating method and the inkjet method are usable.

### Cathode

It is preferable to use metal, an alloy, an electroconductive compound, a mixture thereof, or the like having a small work function (specifically, 3.8 eV or less) for the cathode.

Examples of the material for the cathode include elements belonging to Groups 1 and 2 in the periodic table of the elements, specifically, an alkali metal such as lithium (Li) and cesium (Cs), an alkaline earth metal such as magnesium (Mg), calcium (Ca) and strontium (Sr), alloys (e.g., MgAg and AlLi) including the alkali metal or the alkaline earth metal, a rare earth metal such as europium (Eu) and ytterbium (Yb), and alloys including the rare earth metal.

It should be noted that the vacuum deposition method and the sputtering method are usable for forming the cathode using the alkali metal, alkaline earth metal and the alloy thereof.

Further, when a silver paste is used for the cathode, the coating method and the inkjet method are usable.

By providing the electron injecting layer, various conductive materials such as A1, Ag, ITO, graphene, and indium oxide-tin oxide containing silicon or silicon oxide may be used for forming the cathode regardless of the work function.

The conductive materials can be formed into a film using the sputtering method, inkjet method, spin coating method and the like.

### Hole Injecting Layer

The hole injecting layer is a layer containing a substance exhibiting a high hole injectability.

Examples of the substance exhibiting a high hole injectability include molybdenum oxide, titanium oxide, vanadium oxide, rhenium oxide, ruthenium oxide, chrome oxide, zirconium oxide, hafnium oxide, tantalum oxide, silver oxide, tungsten oxide, and manganese oxide.

In addition, examples of the substance exhibiting a high hole injectability also include aromatic amine compounds such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}-N-phenylamino)biphenyl (abbreviation: DNTPD), 1,3,5-tris[N-(4-diphenylaminophenyl)-N-phenylamino]benzene (abbreviation: DPA3B), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), and 3-[N-(1-naphthyl)-N-(9-phenylcarbazol-3-yl)amino]-9-phenylcarbazole (abbreviation: PCzPCN1), and dipyrazino[2,3-f:20,30-h]quinoxaline-2,3,6,7,10,11-hexacarbonitrile (HAT-CN), which are low-molecular organic compounds.

In addition, a high polymer compound (e.g., oligomer, dendrimer and polymer) is usable as the substance exhibiting a high hole injectability.

Examples of the high-molecule compound include poly(N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl)methacrylamide](abbreviation: PTPDMA), and poly[N,N′-bis(4-butylphenyl)-N,N′-bis(phenyl)benzidine] (abbreviation: Poly-TPD).

Moreover, acid-added high-molecular compounds such as poly(3,4-ethylenedioxythiophene)/poly(styrenesulfonic acid) (PEDOT/PSS) and polyaniline/poly(styrenesulfonic acid) (PAni/PSS) are also usable.

### Hole Transporting Layer

The hole transporting layer is a layer containing a highly hole-transporting substance.

In the organic EL device according to the third exemplary embodiment, the hole transporting layer preferably contains a third compound.

In the organic EL device according to the third exemplary embodiment, the hole transporting layer is preferably provided between the anode and the emitting zone.

In the organic EL device according to the third exemplary embodiment, the hole transporting layer preferably contains the third compound represented by a formula (H1) below or a formula (H2) below.

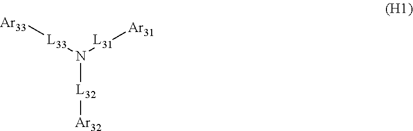

In the formula (H1):
L₃₁, L₃₂, and L₃₃ are each independently a single bond, or a substituted or unsubstituted arylene group having 6 to 18 ring carbon atoms; and
Ar₃₁, Ar₃₂, and Ar₃₃ are each independently a substituted or unsubstituted aryl group having 6 to 30 ring carbon atoms, a substituted or unsubstituted heterocyclic group having 5 to 30 ring atoms, or a group represented by —Si(R_{C1})(R_{C2})(R_{C3});
R_{C1}, R_{C2}, and R_{C3} are each independently a substituted or unsubstituted aryl group having 6 to 30 ring carbon atoms;
when a plurality of R_{C1} are present, the plurality of R_{C1} are mutually the same or different;
when a plurality of R_{C2} are present, the plurality of R_{C2} are mutually the same or different; and
when a plurality of R_{C3} are present, the plurality of R_{C3} are mutually the same or different.

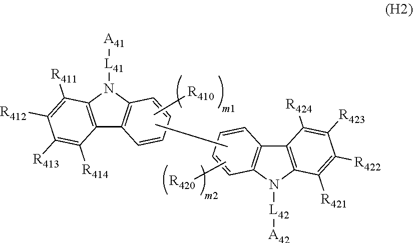

In the formula (H2):
A₄₁ and A₄₂ are each independently a substituted or unsubstituted aryl group having 6 to 30 ring carbon atoms, or a substituted or unsubstituted heterocyclic group having 5 to 30 ring atoms;
at least one combination of adjacent two or more of R₄₁₀ to R₄₁₄ are mutually bonded to form a substituted or unsubstituted monocyclic ring, mutually bonded to form a substituted or unsubstituted fused ring, or not mutually bonded;
at least one combination of adjacent two or more of R₄₂₀ to R₄₂₄ are mutually bonded to form a substituted or unsubstituted monocyclic ring, mutually bonded to form a substituted or unsubstituted fused ring, or not mutually bonded;
R₄₁₀ to R₄₁₄ and R₄₂₀ to R₄₂₄ forming neither the substituted or unsubstituted monocyclic ring nor the substituted or unsubstituted fused ring are each independently a hydrogen atom, a cyano group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a halogen atom, a nitro group, a substituted or unsubstituted aryl group having 6 to 50 ring carbon atoms, or a substituted or unsubstituted heterocyclic group having 5 to 50 ring atoms;
m1 is 3, and three R₄₁₀ are mutually the same or different;
m2 is 3, and three R₄₂₀ are mutually the same or different; and
L₄₁ and L₄₂ are each independently a single bond, a substituted or unsubstituted arylene group having 6 to 30 ring carbon atoms, or a substituted or unsubstituted divalent heterocyclic group having 5 to 30 ring atoms.

In the third compound represented by the formula (H2), R₉₀₁, R₉₀₂, R₉₀₃, and R₉₀₄ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different; and
when a plurality of R₉₀₄ are present, the plurality of R₉₀₄ are mutually the same or different.

In the organic EL device according to the third exemplary embodiment, the hole transporting layer also preferably contains a compound represented by a formula (H3) below as the third compound.

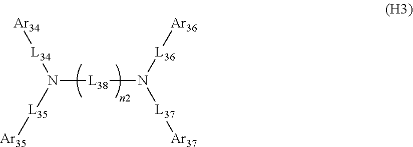

In the formula (H3):
L₃₄, L₃₅, L₃₆, and L₃₇ are each independently a single bond, or a substituted or unsubstituted arylene group having 6 to 18 ring carbon atoms; and
n2 is 1, 2, 3, or 4;
when n2 is 1, L₃₈ is a substituted or unsubstituted arylene group having 6 to 18 ring carbon atoms;
when n2 is 2, 3, or 4, the plurality of L₃₈ are mutually the same or different;
when n2 is 2, 3, or 4, the plurality of L₃₈ are mutually bonded to form a substituted or unsubstituted monocyclic ring, mutually bonded to form a substituted or unsubstituted fused ring, or not mutually bonded;
L₃₈ forming neither the substituted or unsubstituted monocyclic ring nor the substituted or unsubstituted fused ring is a substituted or unsubstituted arylene group having 6 to 18 ring carbon atoms;
Ar₃₄, Ar₃₅, Ar₃₆, and Ar₃₇ are each independently a substituted or unsubstituted aryl group having 6 to 30 ring carbon atoms, a substituted or unsubstituted heterocyclic group having 5 to 30 ring atoms, or a group represented by —Si(R_{C1})(R_{C2})(R_{C3});
R_{C1}, R_{C2}, and R_{C3} are each independently a substituted or unsubstituted aryl group having 6 to 30 ring carbon atoms;
when a plurality of R_{C1} are present, the plurality of R_{C1} are mutually the same or different;
when a plurality of R_{C2} are present, the plurality of R_{C2} are mutually the same or different; and
when a plurality of R_{C3} are present, the plurality of R_{C3} are mutually the same or different.

In the organic EL device according to the third exemplary embodiment, at least one of Ar₃₁, Ar₃₂, or Ar₃₃ in the third compound is also preferably a group represented by a formula (H11) below.

In the organic EL device according to the third exemplary embodiment, at least one of Ar₃₄, Aram, Ar₃₆, or Ar₃ in the third compound is also preferably a group represented by the formula (H11) below.

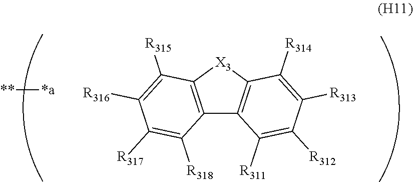

In the formula (H11):
X₃ is an oxygen atom, a sulfur atom, NR₃₁₉, or C(R₃₂₀)(R₃₂₁);
a combination of adjacent two or more of R₃₁₁ to R₃₁₈ are mutually bonded to form a substituted or unsubstituted monocyclic ring, mutually bonded to form a substituted or unsubstituted fused ring, or not mutually bonded;
a combination of R₃₂₀ and R₃₂₁ are mutually bonded to form a substituted or unsubstituted monocyclic ring, mutually bonded to form a substituted or unsubstituted fused ring, or not mutually bonded;
one of R₃₁₁ to R₃₂₁ is a single bond bonded to *a, a carbon atom forming a ring skeleton of the substituted or unsubstituted monocyclic ring or the substituted or unsubstituted fused ring formed by mutual bonding of a combination of adjacent two or more of R₃₁₁ to R₃₁₈ is bonded to *a with a single bond, or a carbon atom forming a ring skeleton of the substituted or unsubstituted monocyclic ring or the substituted or unsubstituted fused ring formed by mutual bonding of a combination of R₃₂₀ and R₃₂₁ is bonded to *a with a single bond;
R₃₁₁ to R₃₁₈ forming neither the substituted or unsubstituted monocyclic ring nor the substituted or unsubstituted fused ring and not being the single bond bonded to *a are each independently a hydrogen atom, a substituted or unsubstituted alkyl group having 1 to 6 carbon atoms, a substituted or unsubstituted aryl group having 6 to 12 ring carbon atoms, or a substituted or unsubstituted heterocyclic group having 5 to 10 ring atoms;
R₃₁₉ not being the single bond bonded to *a is a hydrogen atom, a substituted or unsubstituted alkyl group having 1 to 6 carbon atoms, or a substituted or unsubstituted aryl group having 6 to 12 ring carbon atoms;
R₃₂₀ and R₃₂₁ not being the single bond bonded to *a and forming neither the substituted or unsubstituted monocyclic ring nor the substituted or unsubstituted fused ring are each independently a hydrogen atom, a substituted or unsubstituted alkyl group having 1 to 6 carbon atoms, or a substituted or unsubstituted aryl group having 6 to 12 ring carbon atoms; and
each ** is independently a bonding position to L₃₁, L₃₂, or L₃₃, a bonding position to L₃₄, L₃₅, L₃₆, or L₃₇, or a bonding position to the nitrogen atom of an amino group.

In at least one group represented by the formula (H11) in the third compound, it is also preferable that at least one combination of adjacent two or more of R₃₁₁ to R₃₁₈ are mutually bonded to form a substituted or unsubstituted monocyclic ring or a substituted or unsubstituted fused ring.

In at least one group represented by the formula (H11) in the third compound, it is also preferable that at least one combination of adjacent two or more of R₃₁₁ to R₃₁₈ are mutually bonded to form a substituted or unsubstituted benzene ring.

In at least one group represented by the formula (H11) in the third compound, it is also preferable that one or two combinations of adjacent two or more of R₃₁₁ to R₃₁₈ are mutually bonded to form one or two substituted or unsubstituted benzene rings.

In at least one group represented by the formula (H11) in the third compound, it is also preferable that none of the combinations of adjacent two or more of R₃₁₁ to R₃₁₈ are mutually bonded.

In the organic EL device according to the third exemplary embodiment, the third compound is also preferably at least one amine compound selected from the group consisting of a monoamine compound having one substituted or unsubstituted amino group in a molecule, a diamine compound having two substituted or unsubstituted amino groups in a molecule, a triamine compound having three substituted or unsubstituted amino groups in a molecule, and a tetraamine compound having four substituted or unsubstituted amino groups in a molecule.

In the compound represented by the formula (H1) and the compound represented by the formula (H3), the substituent for the “substituted or unsubstituted” group is also preferably not a group represented by —N(R_{C6})(R_{C7}).

In the group represented by —N(R_{C6})(R_{C7}), R_{C6} and R_{C7} are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms.

In the organic EL device according to the third exemplary embodiment, the third compound is also preferably at least one amine compound selected from the group consisting of a monoamine compound and a diamine compound.

In the organic EL device according to the third exemplary embodiment, the third compound is also preferably a monoamine compound.

In the organic EL device according to the third exemplary embodiment, an aromatic amine compound, a carbazole derivative, an anthracene derivative, and the like are usable for the hole transporting layer.

Specific examples of a material for the hole transporting layer include 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4-phenyl-4′-(9-phenylfluorene-9-yl)triphenylamine (abbreviation: BAFLP), 4,4′-bis[N-(9,9-dimethylfluorene-2-yl)-N-phenylamino]biphenyl (abbreviation: DFLDPBi), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluorene-2-yl)-N-phenylamino]biphenyl (abbreviation: BSPB).

The above-described substances mostly have a hole mobility of 10⁻⁶ cm²/(V·s) or more.

For the hole transporting layer, a carbazole derivative such as CBP, 9-[4-(N-carbazolyl)]phenyl-10-phenylanthracene (CzPA), and 9-phenyl-3-[4-(10-phenyl-9-anthryl)phenyl]-9H-carbazole (PCzPA) and an anthracene derivative such as t-BuDNA, DNA, and DPAnth may be used.

A high polymer compound such as poly(N-vinylcarbazole) (abbreviation: PVK) and poly(4-vinyltriphenylamine) (abbreviation: PVTPA) is also usable.

However, in addition to the above substances, any substance exhibiting a higher hole transportability than an electron transportability may be used.

It should be noted that the layer containing the substance exhibiting a high hole transportability may be not only a single layer but also a laminate of two or more layers formed of the above substance(s).

### Specific Examples of Third Compound

Specific examples of the third compound include the following compounds.

However, the invention is not limited to these specific examples of the third compound.

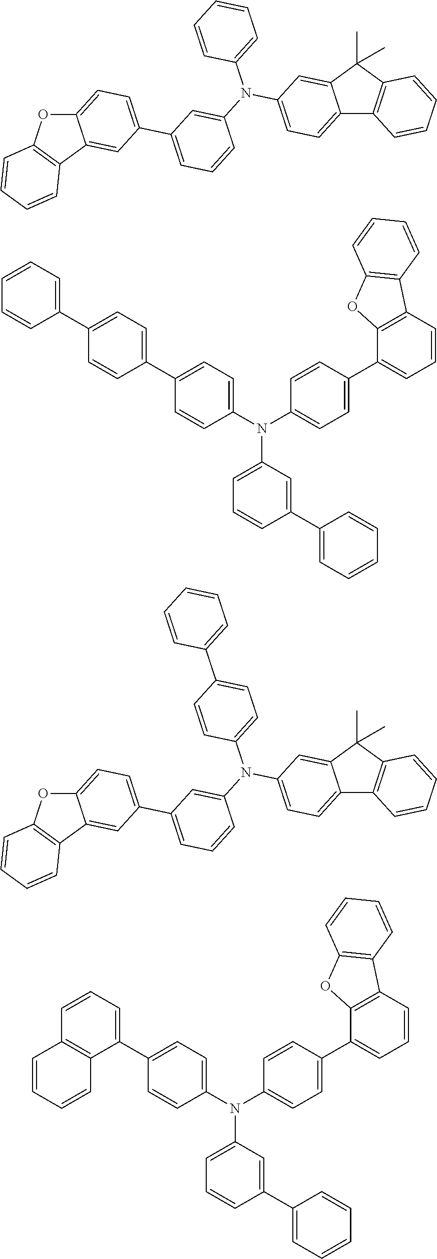 [SMILES: CC1(C)c2ccccc2-c2ccc(N(c3ccccc3)c3cccc(-c4ccc5oc6ccccc6c5c4)c3)cc21 ; c1ccc(-c2ccc(-c3ccc(N(c4ccc(-c5cccc6c5oc5ccccc56)cc4)c4cccc(-c5ccccc5)c4)cc3)cc2)cc1 ; CC1(C)c2ccccc2-c2ccc(N(c3ccc(-c4ccccc4)cc3)c3cccc(-c4ccc5oc6ccccc6c5c4)c3)cc21 ; c1ccc(-c2cccc(N(c3ccc(-c4cccc5ccccc45)cc3)c3ccc(-c4cccc5c4oc4ccccc45)cc3)c2)cc1]

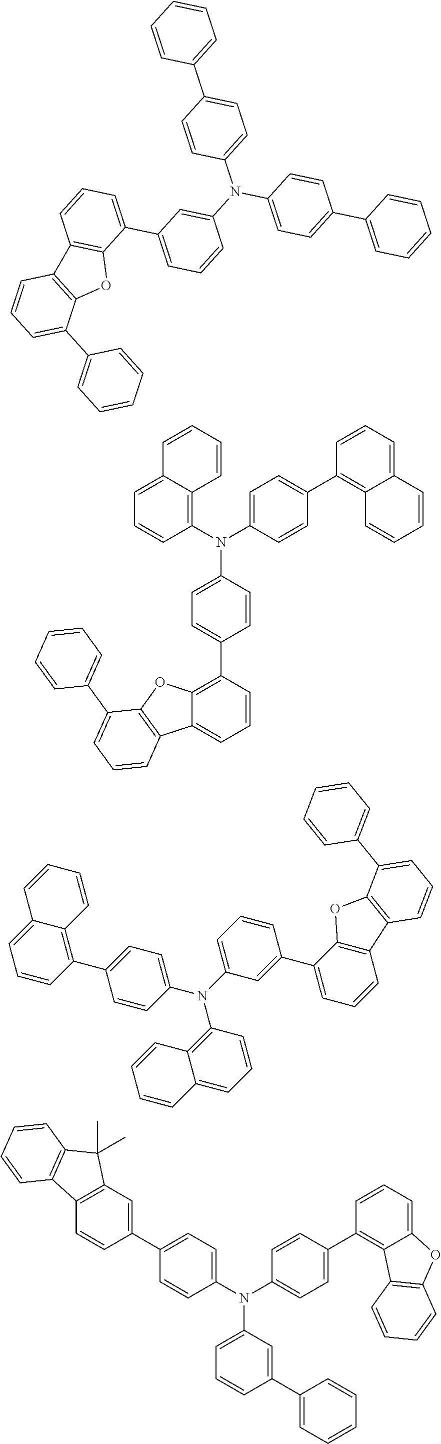

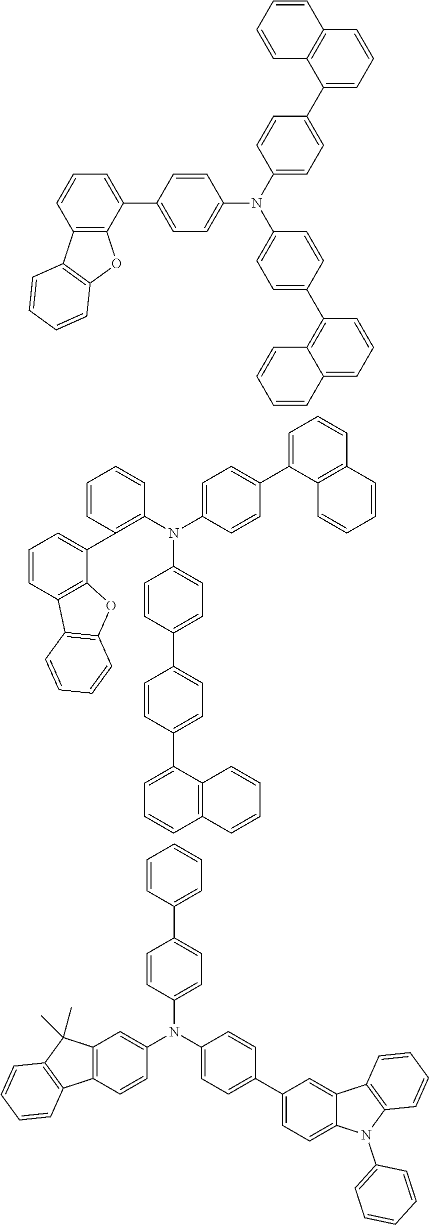

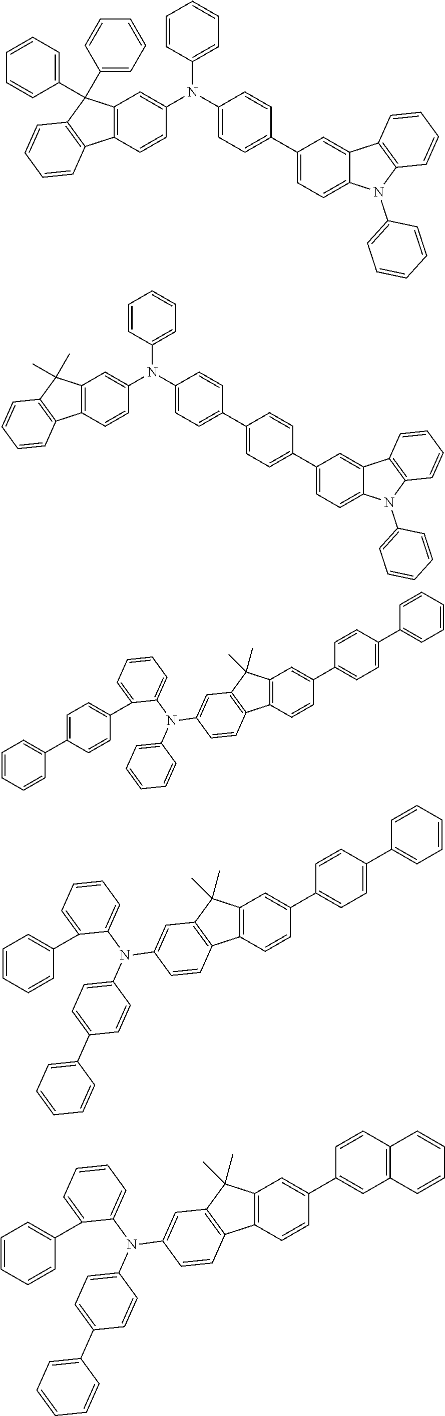

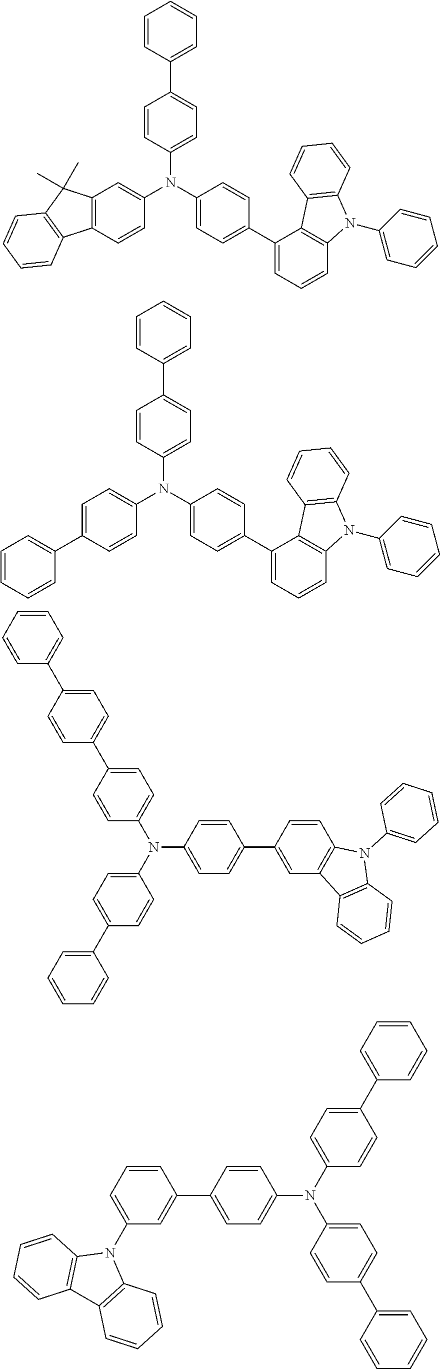

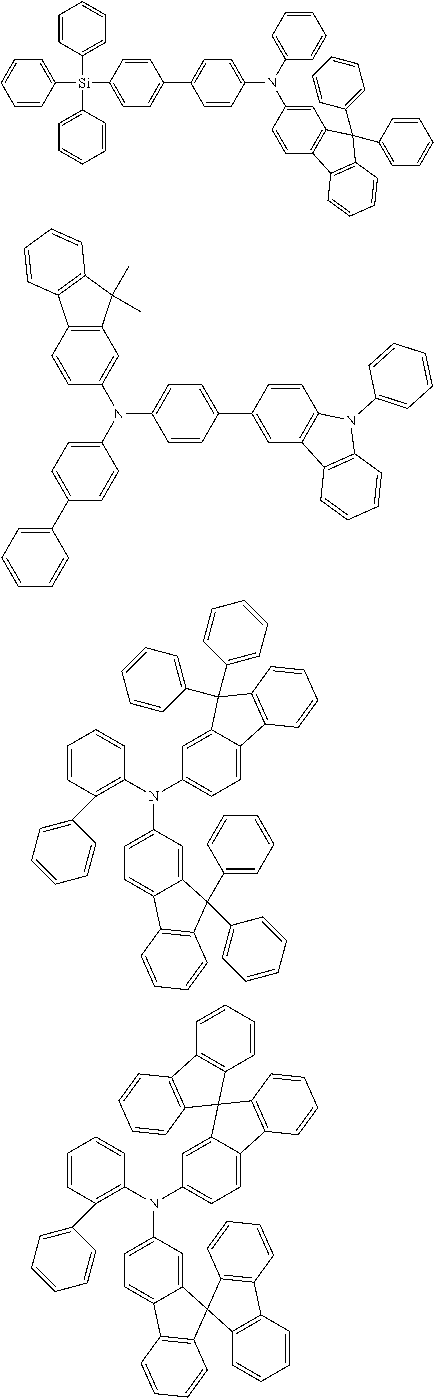

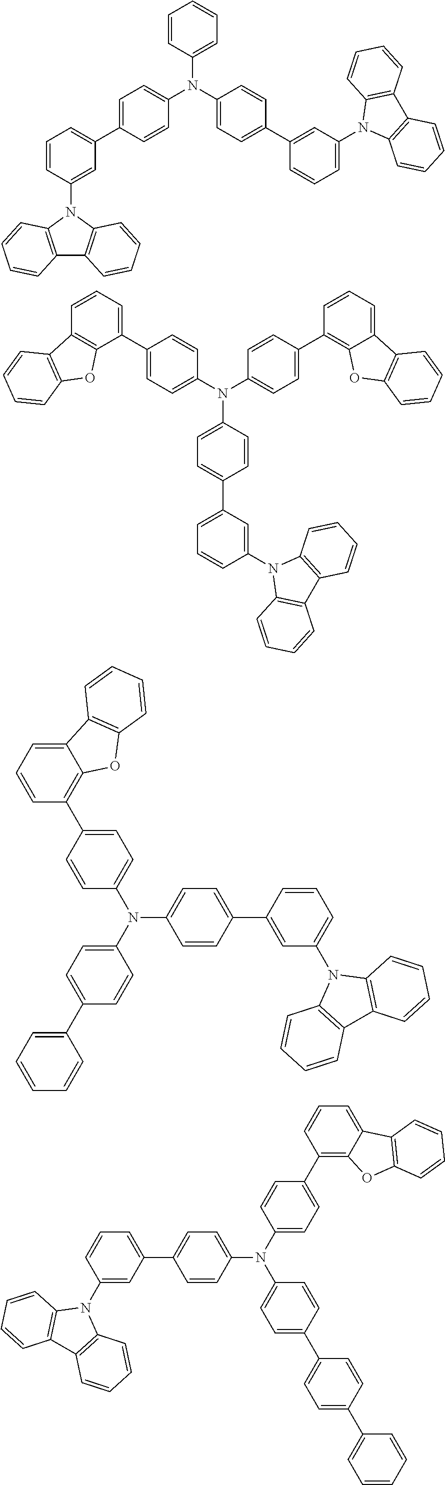

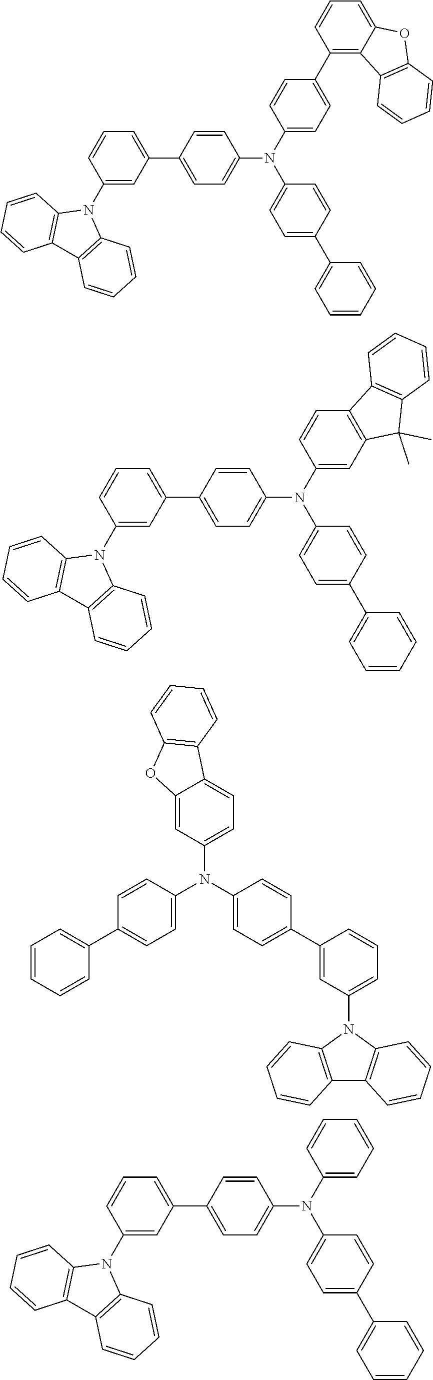

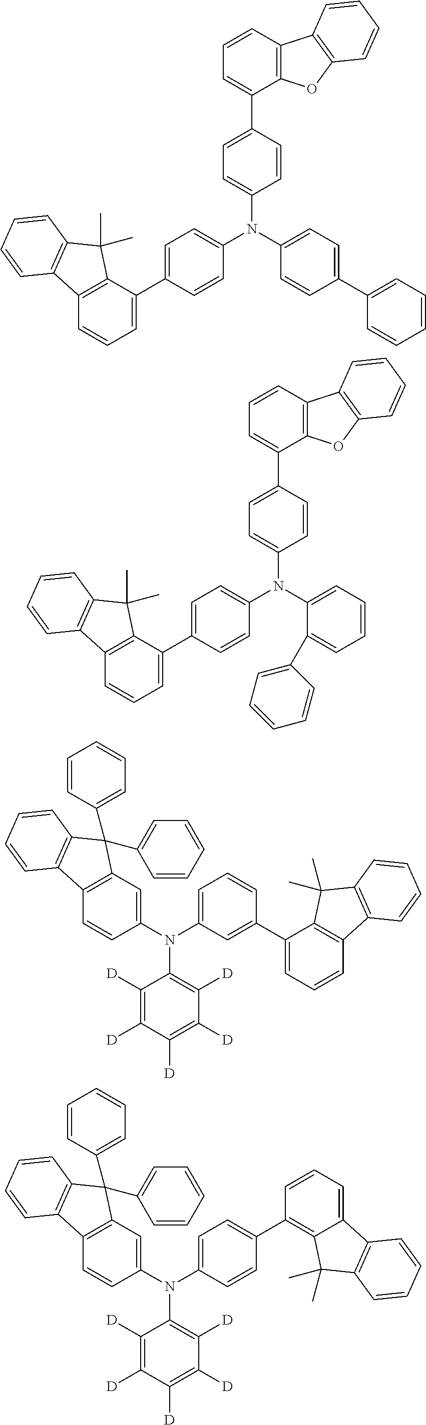

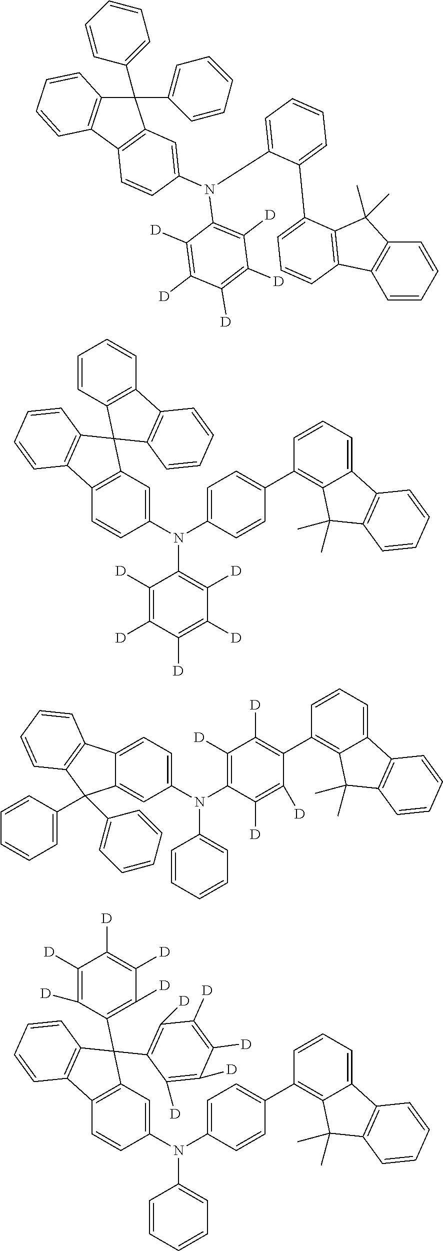 [SMILES: [2H]c1c([2H])c([2H])c(N(c2ccc3c(c2)C(c2ccccc2)(c2ccccc2)c2ccccc2-3)c2ccccc2-c2cccc3c2C(C)(C)c2ccccc2-3)c([2H])c1[2H] ; [2H]c1c([2H])c([2H])c(N(c2ccc(-c3cccc4c3C(C)(C)c3ccccc3-4)cc2)c2ccc3c(c2)C2(c4ccccc4-c4ccccc42)c2ccccc2-3)c([2H])c1[2H] ; [2H]c1c([2H])c(N(c2ccccc2)c2ccc3c(c2)C(c2ccccc2)(c2ccccc2)c2ccccc2-3)c([2H])c([2H])c1-c1cccc2c1C(C)(C)c1ccccc1-2 ; [2H]c1c([2H])c([2H])c(C2(c3c([2H])c([2H])c([2H])c([2H])c3[2H])c3ccccc3-c3ccc(N(c4ccccc4)c4ccc(-c5cccc6c5C(C)(C)c5ccccc5-6)cc4)cc32)c([2H])c1[2H]]

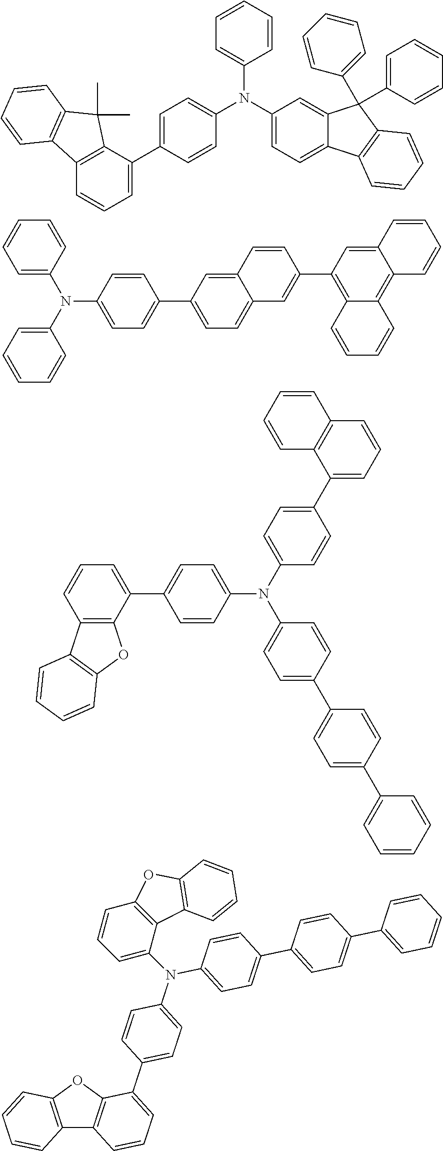

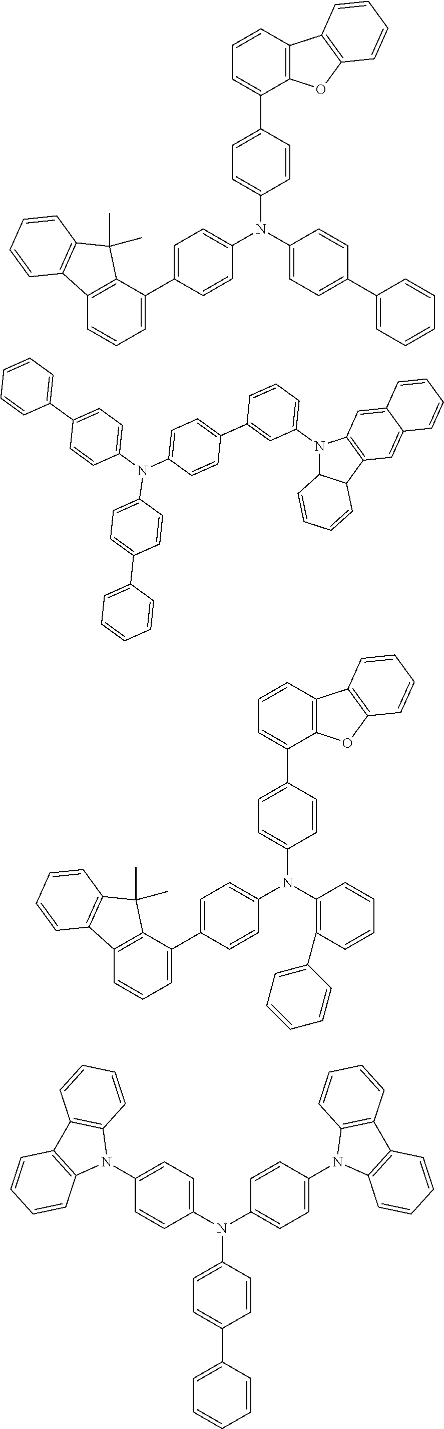

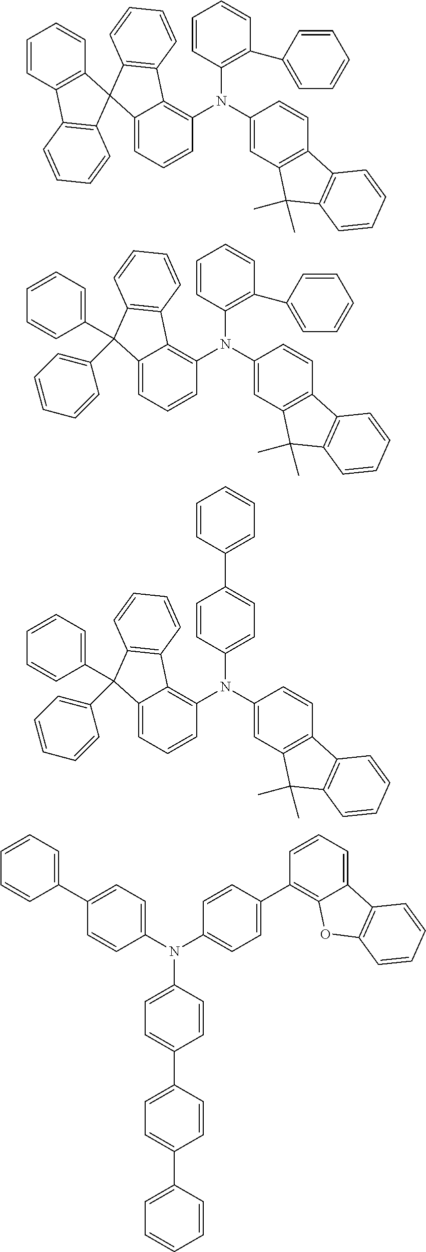

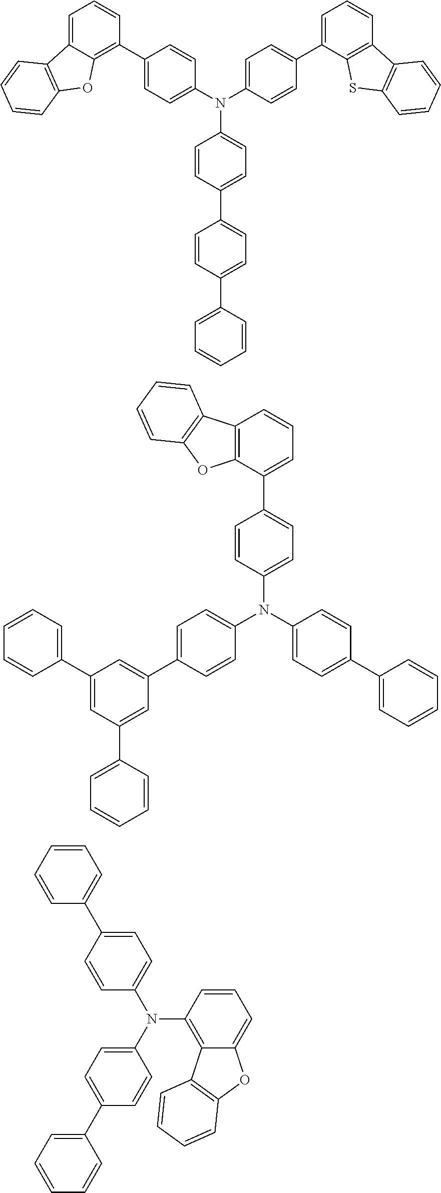

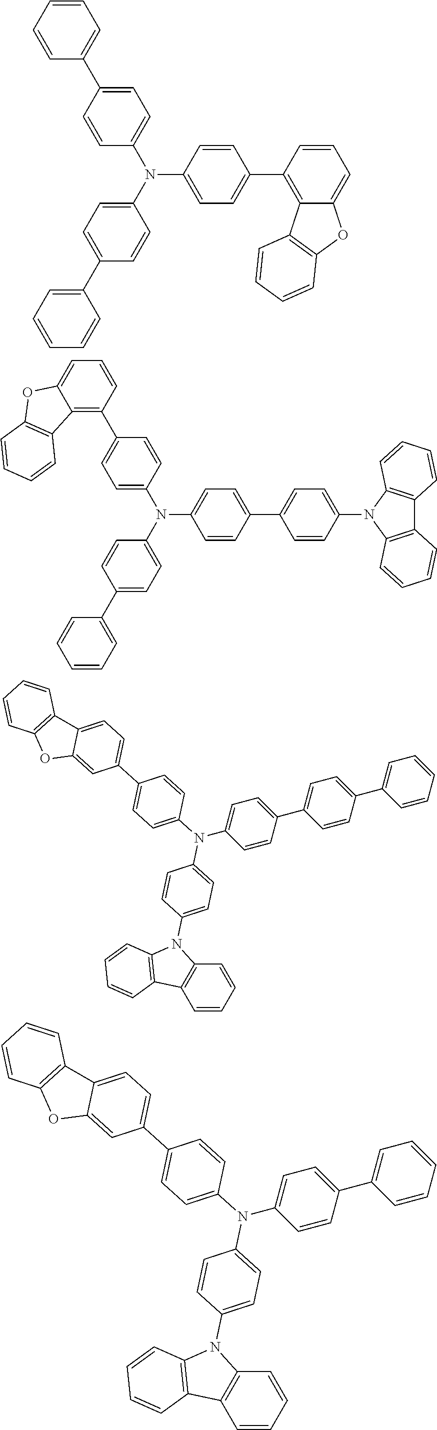

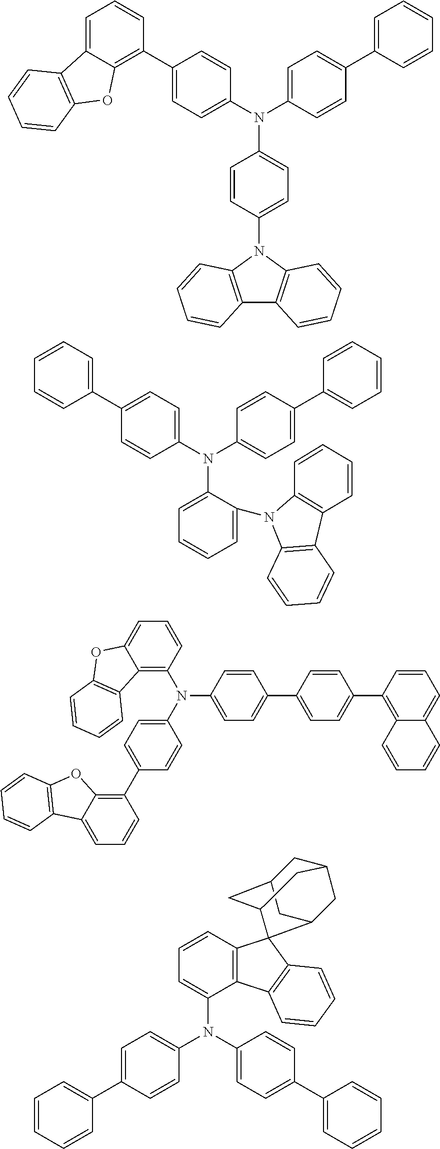

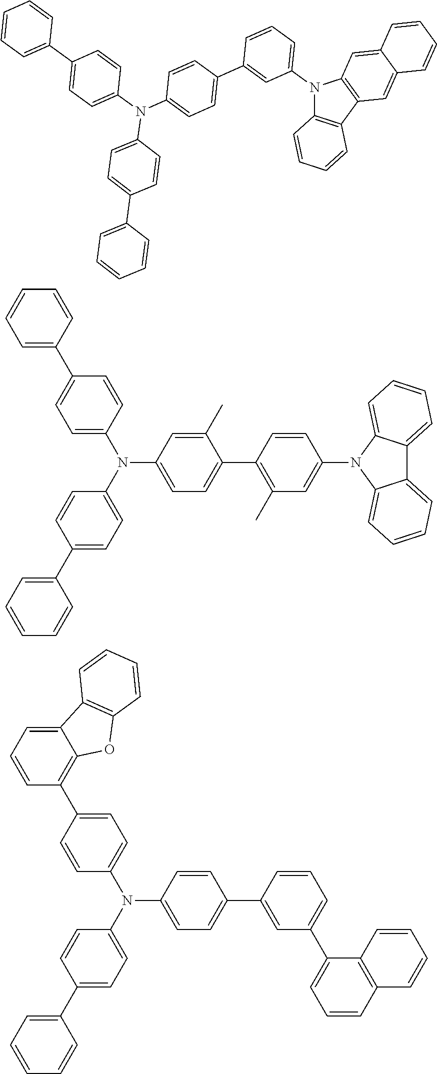

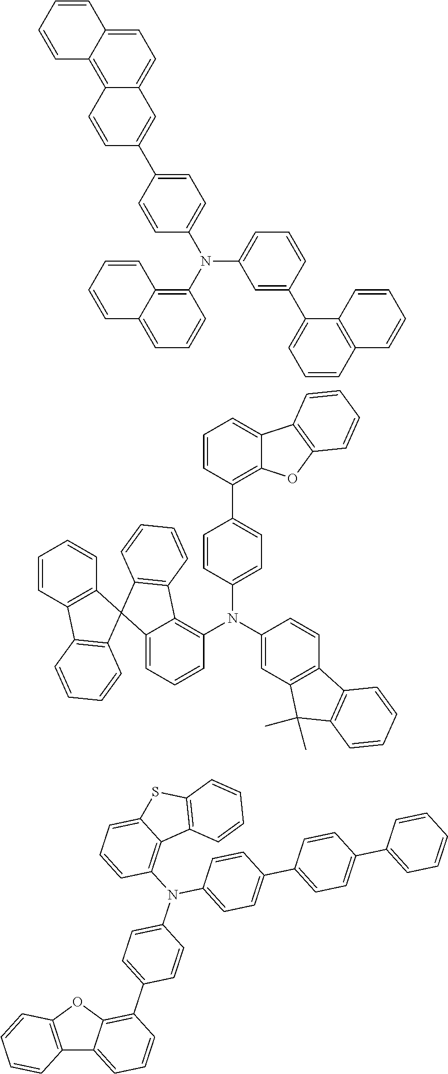

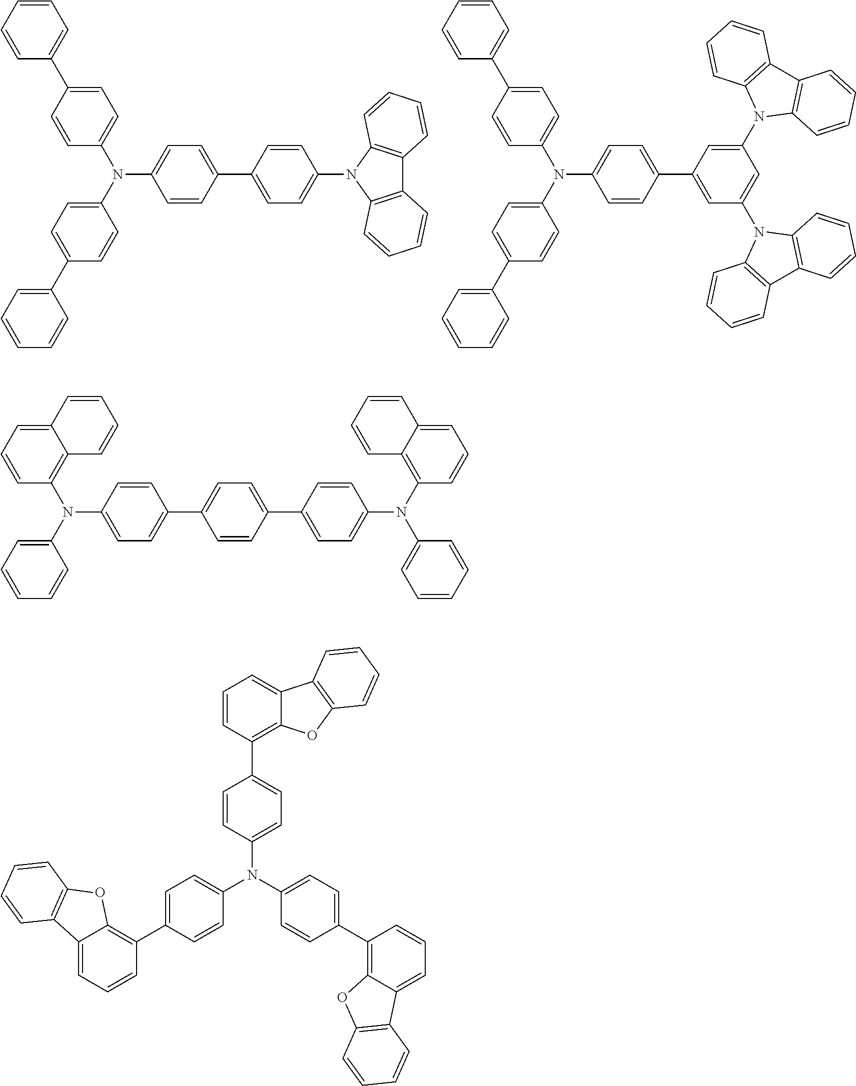 [SMILES: c1ccc(-c2ccc(N(c3ccc(-c4ccccc4)cc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4cc(-n5c6ccccc6c6ccccc65)cc(-n5c6ccccc6c6ccccc65)c4)cc3)cc2)cc1 ; c1ccc(N(c2ccc(-c3ccc(-c4ccc(N(c5ccccc5)c5cccc6ccccc56)cc4)cc3)cc2)c2cccc3ccccc23)cc1 ; c1ccc2c(c1)oc1c(-c3ccc(N(c4ccc(-c5cccc6c5oc5ccccc56)cc4)c4ccc(-c5cccc6c5oc5ccccc56)cc4)cc3)cccc12]

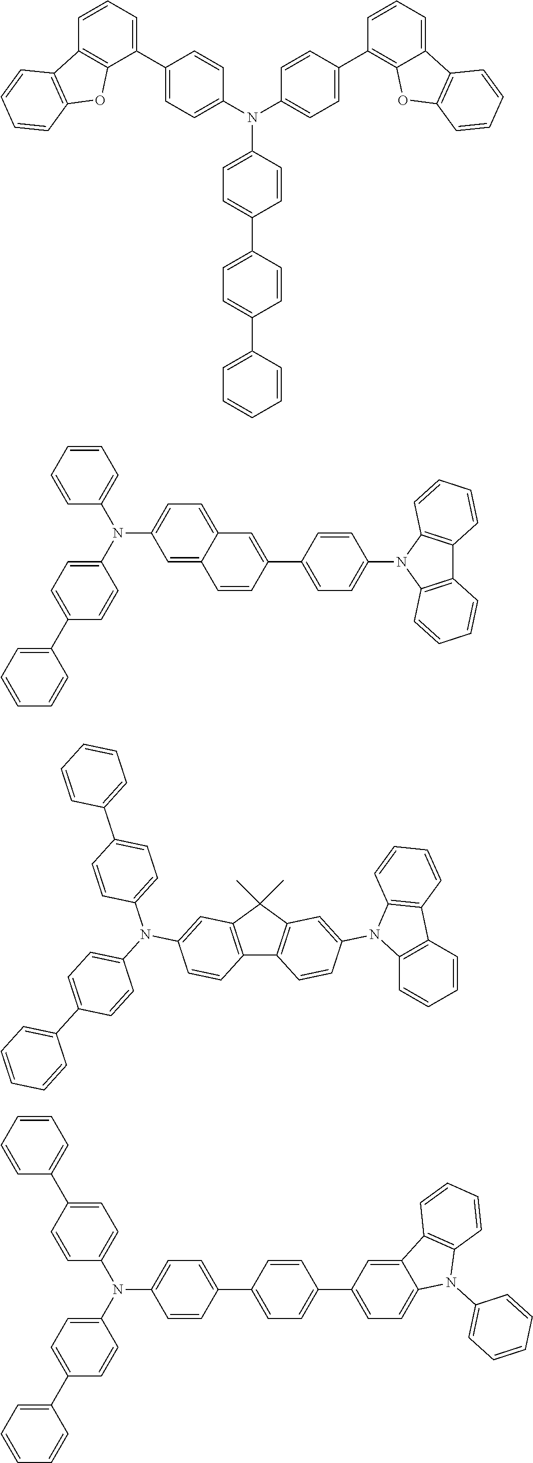

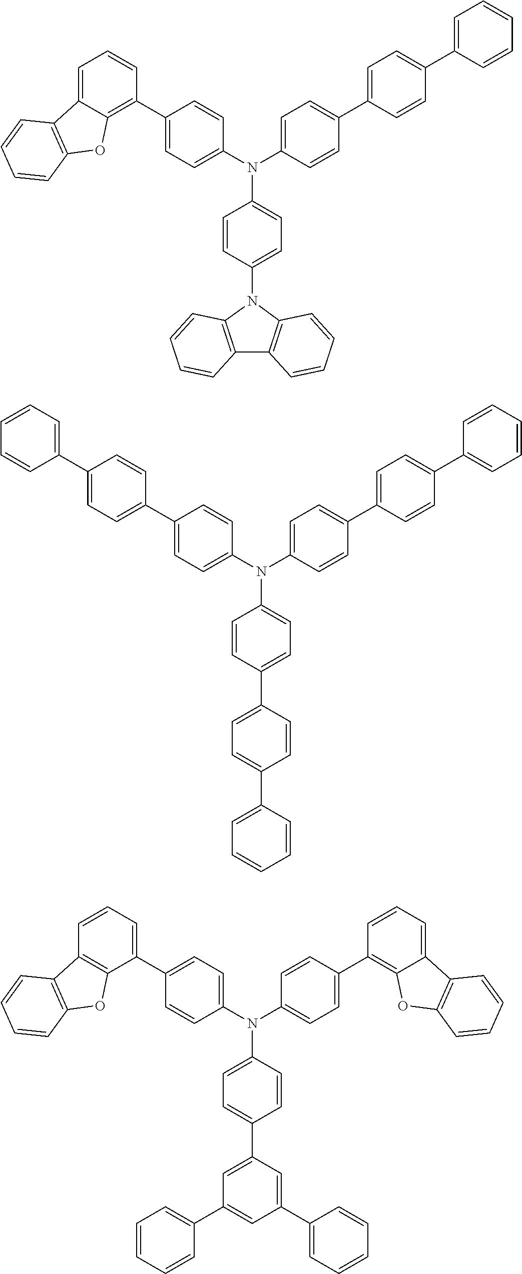 [SMILES: c1ccc(-c2ccc(-c3ccc(N(c4ccc(-c5cccc6c5oc5ccccc56)cc4)c4ccc(-n5c6ccccc6c6ccccc65)cc4)cc3)cc2)cc1 ; c1ccc(-c2ccc(-c3ccc(N(c4ccc(-c5ccc(-c6ccccc6)cc5)cc4)c4ccc(-c5ccc(-c6ccccc6)cc5)cc4)cc3)cc2)cc1 ; c1ccc(-c2cc(-c3ccccc3)cc(-c3ccc(N(c4ccc(-c5cccc6c5oc5ccccc56)cc4)c4ccc(-c5cccc6c5oc5ccccc56)cc4)cc3)c2)cc1]

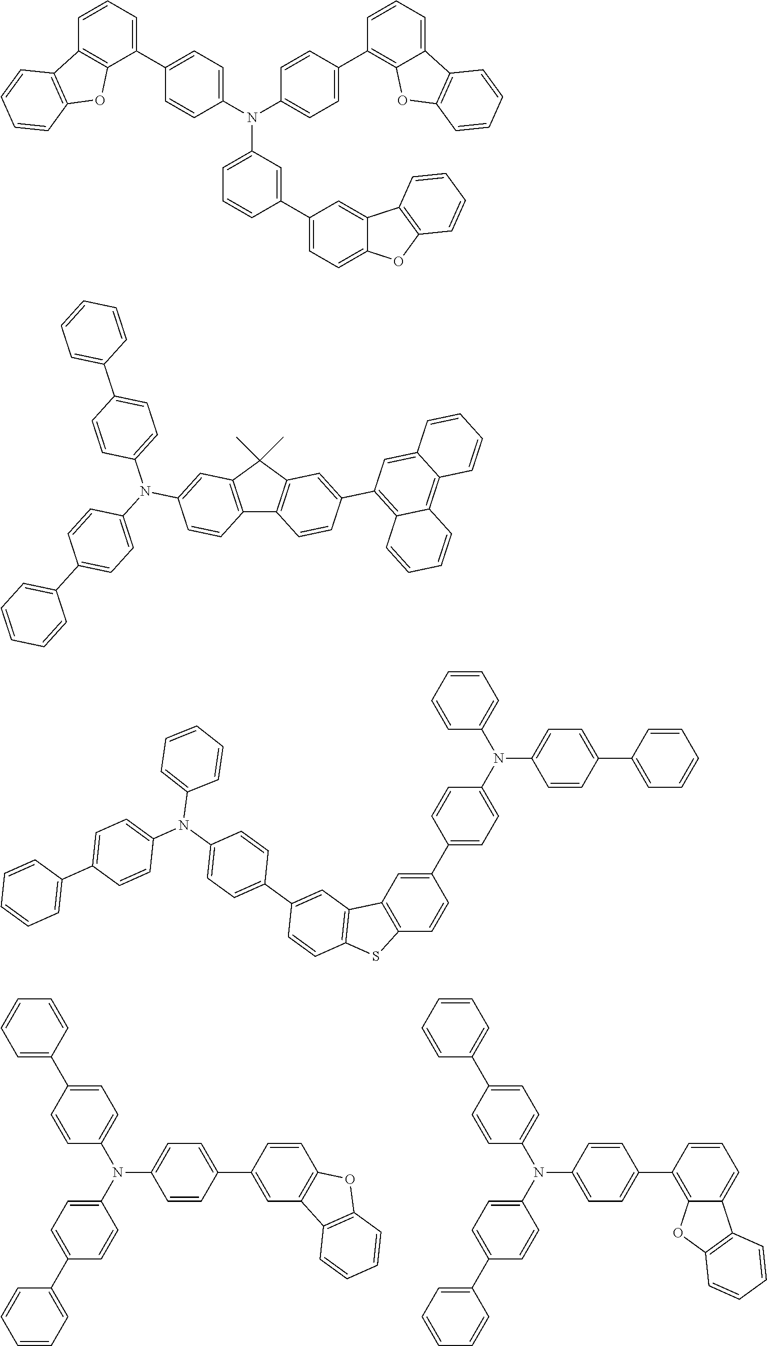 [SMILES: c1cc(-c2ccc3oc4ccccc4c3c2)cc(N(c2ccc(-c3cccc4c3oc3ccccc34)cc2)c2ccc(-c3cccc4c3oc3ccccc34)cc2)c1 ; CC1(C)c2cc(-c3cc4ccccc4c4ccccc34)ccc2-c2ccc(N(c3ccc(-c4ccccc4)cc3)c3ccc(-c4ccccc4)cc3)cc21 ; c1ccc(-c2ccc(N(c3ccccc3)c3ccc(-c4ccc5sc6ccc(-c7ccc(N(c8ccccc8)c8ccc(-c9ccccc9)cc8)cc7)cc6c5c4)cc3)cc2)cc1 ; c1ccc(-c2ccc(N(c3ccc(-c4ccccc4)cc3)c3ccc(-c4ccc5oc6ccccc6c5c4)cc3)cc2)cc1 ; c1ccc(-c2ccc(N(c3ccc(-c4ccccc4)cc3)c3ccc(-c4cccc5c4oc4ccccc45)cc3)cc2)cc1]

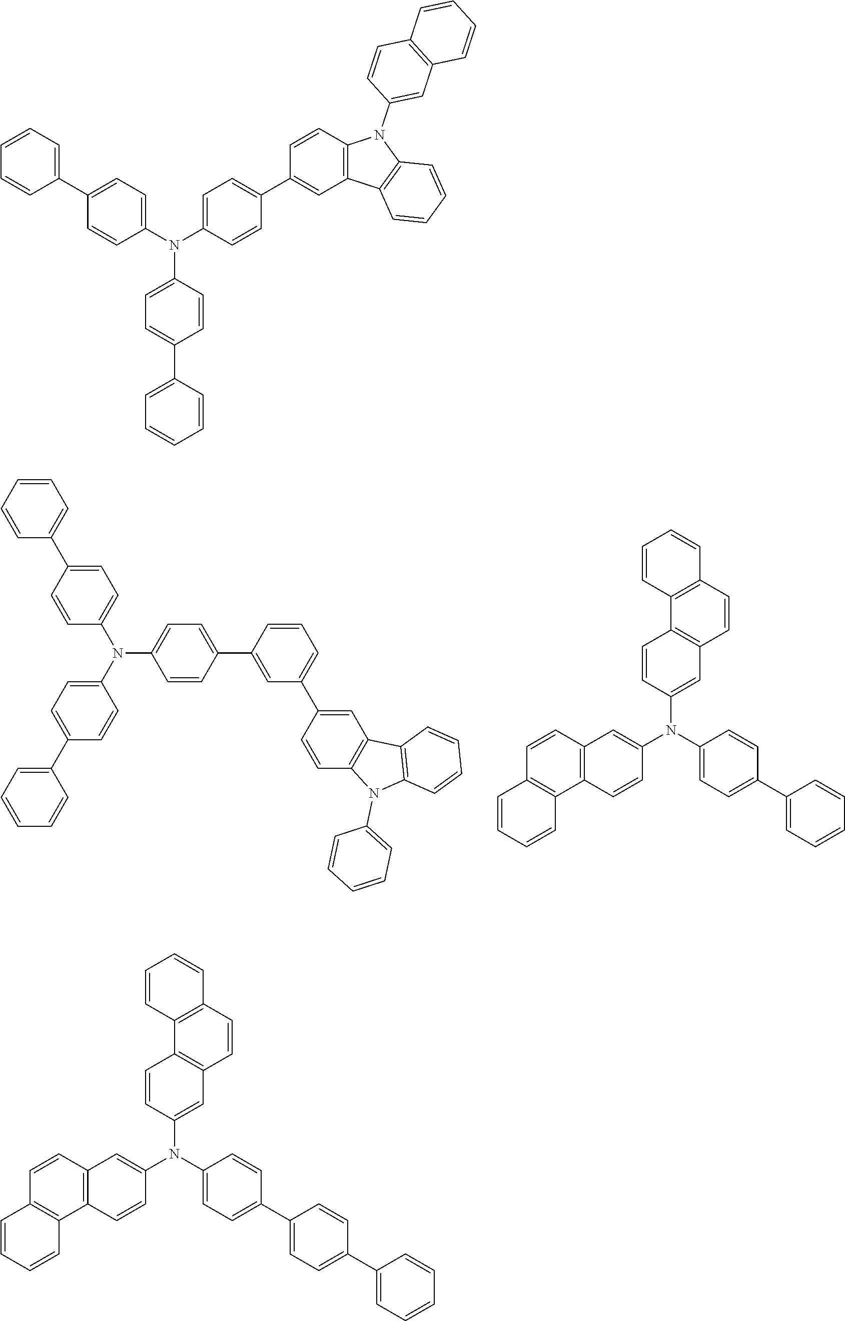

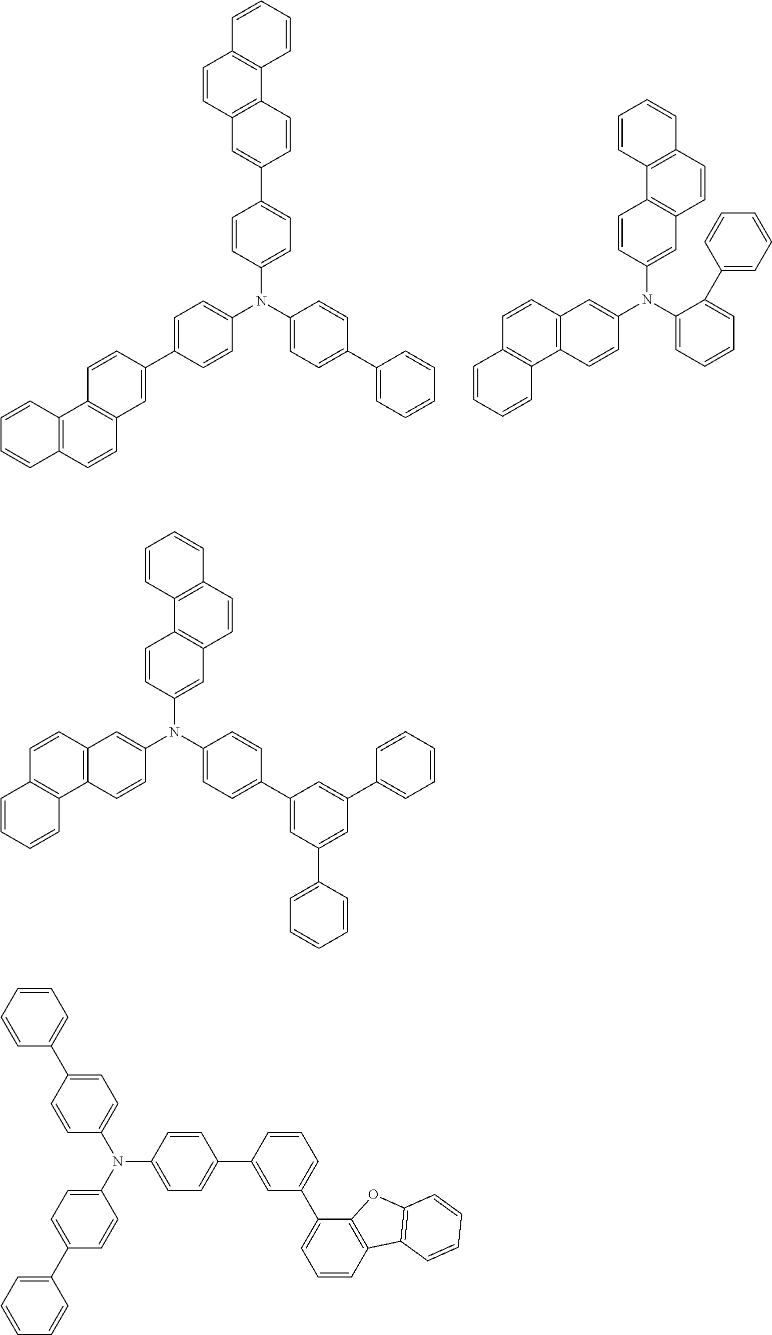

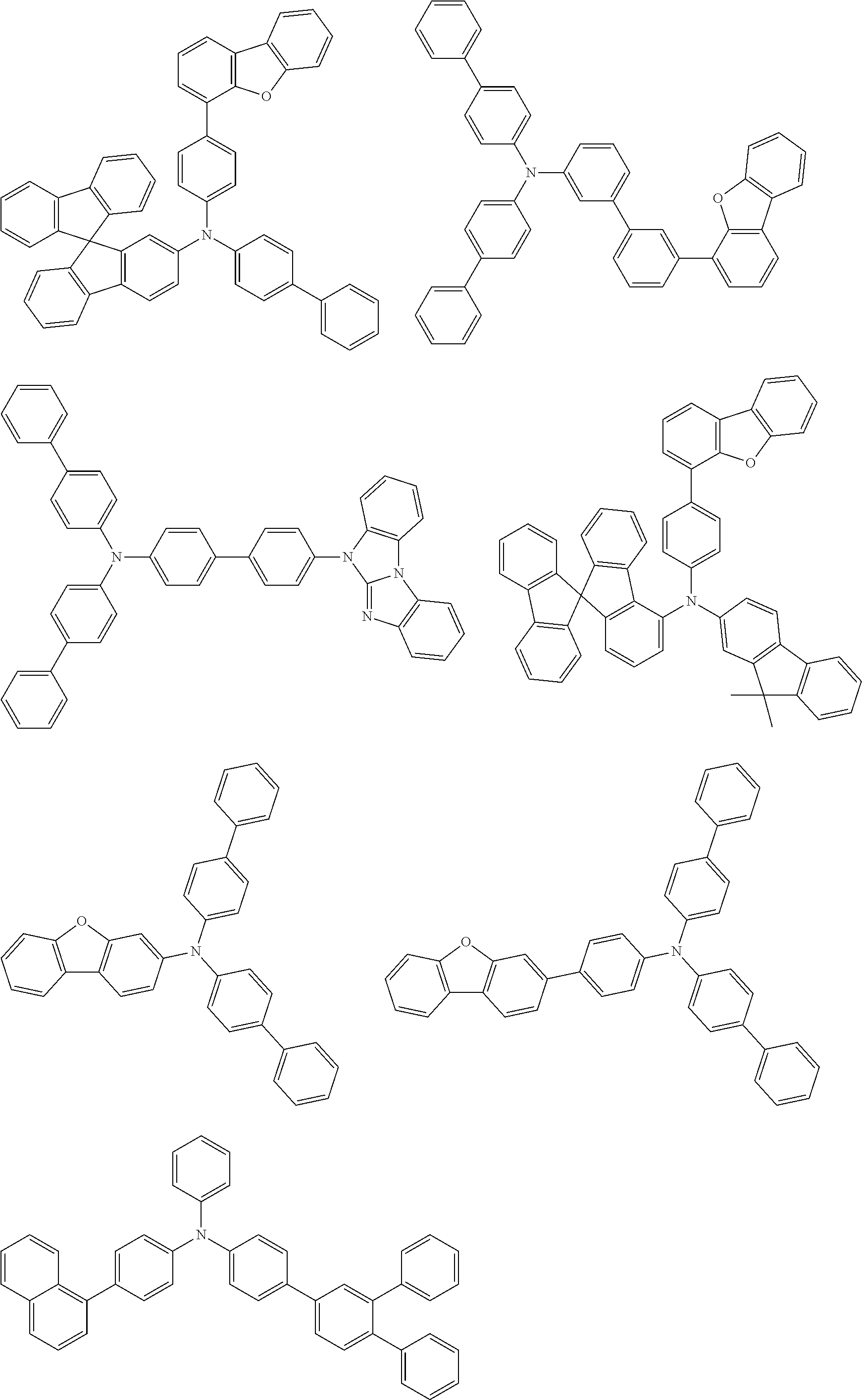

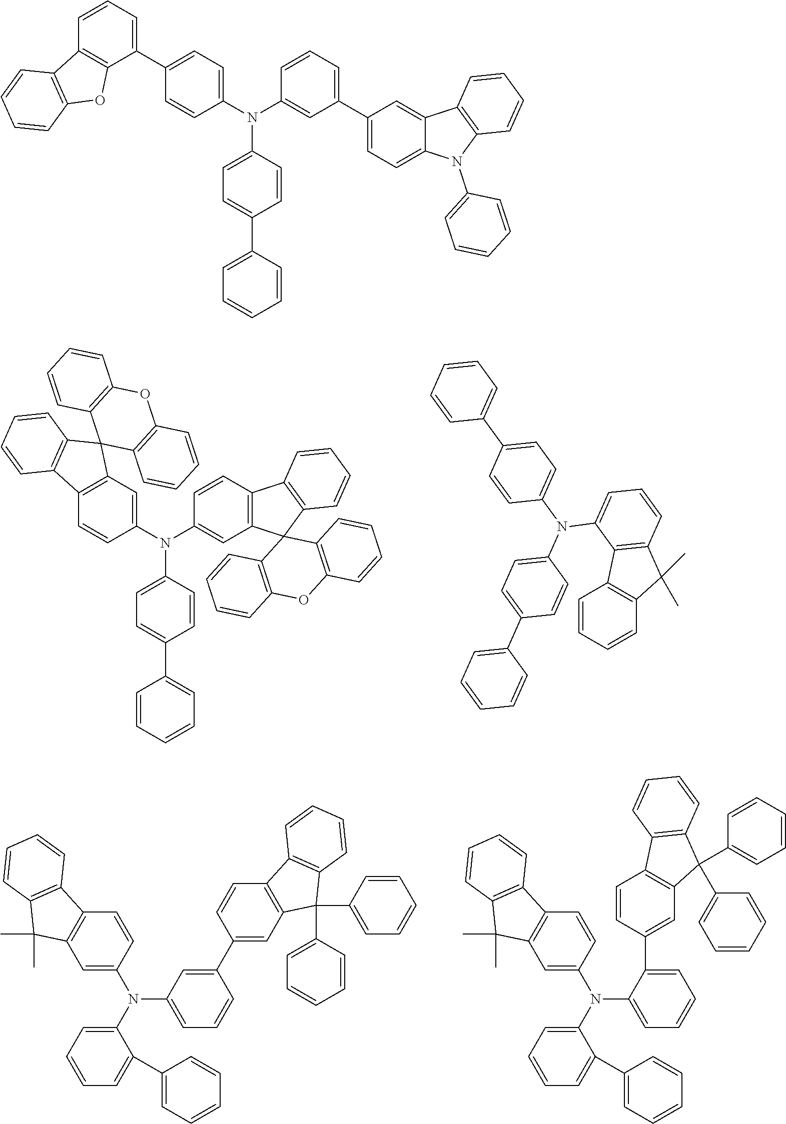 [SMILES: c1ccc(-c2ccc(N(c3ccc(-c4cccc5c4oc4ccccc45)cc3)c3cccc(-c4ccc5c(c4)c4ccccc4n5-c4ccccc4)c3)cc2)cc1 ; c1ccc(-c2ccc(N(c3ccc4c(c3)C3(c5ccccc5Oc5ccccc53)c3ccccc3-4)c3ccc4c(c3)C3(c5ccccc5Oc5ccccc53)c3ccccc3-4)cc2)cc1 ; CC1(C)c2ccccc2-c2c(N(c3ccc(-c4ccccc4)cc3)c3ccc(-c4ccccc4)cc3)cccc21 ; CC1(C)c2ccccc2-c2ccc(N(c3cccc(-c4ccc5c(c4)C(c4ccccc4)(c4ccccc4)c4ccccc4-5)c3)c3ccccc3-c3ccccc3)cc21 ; CC1(C)c2ccccc2-c2ccc(N(c3ccccc3-c3ccccc3)c3ccccc3-c3ccc4c(c3)C(c3ccccc3)(c3ccccc3)c3ccccc3-4)cc21]

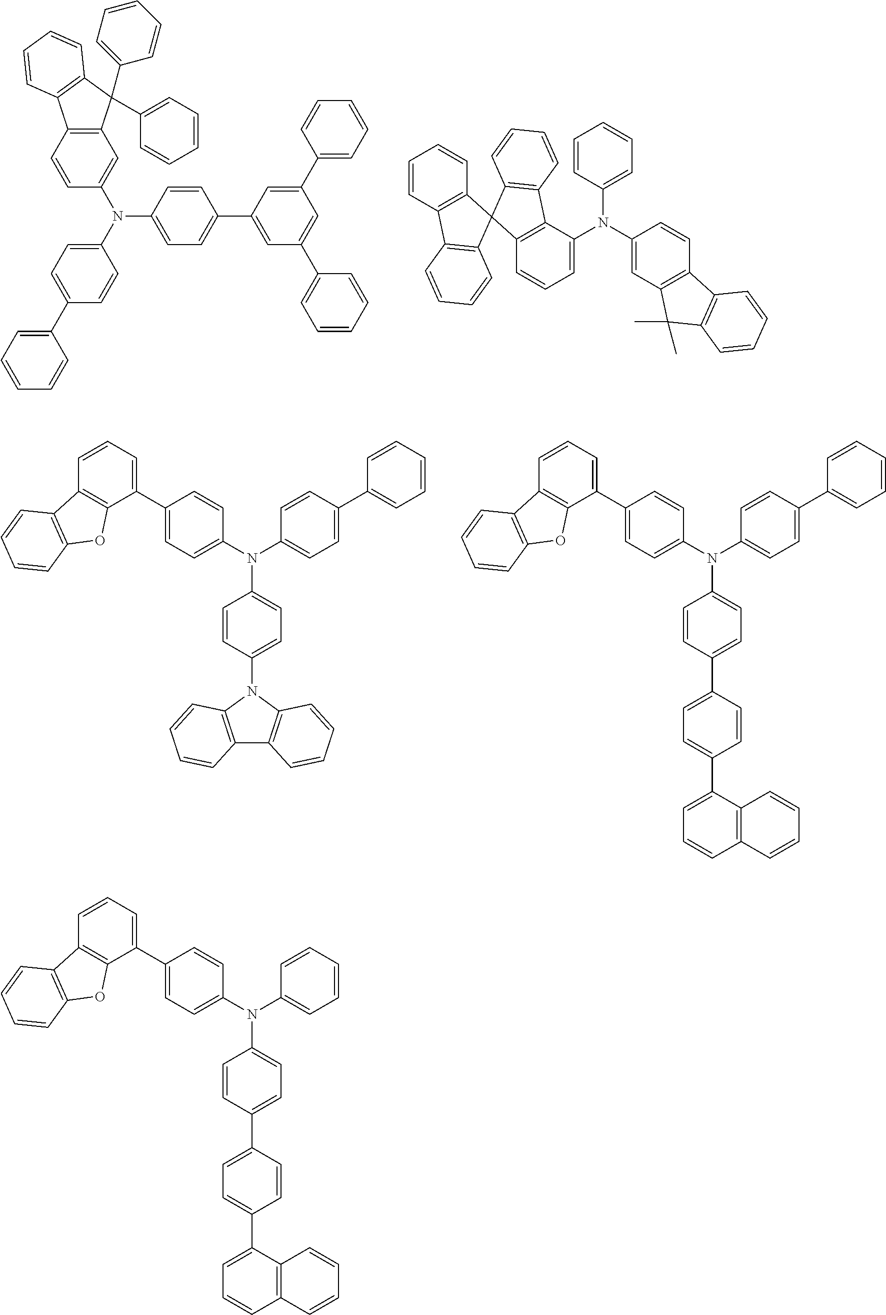 [SMILES: c1ccc(-c2ccc(N(c3ccc(-c4cc(-c5ccccc5)cc(-c5ccccc5)c4)cc3)c3ccc4c(c3)C(c3ccccc3)(c3ccccc3)c3ccccc3-4)cc2)cc1 ; CC1(C)c2ccccc2-c2ccc(N(c3ccccc3)c3cccc4c3-c3ccccc3C43c4ccccc4-c4ccccc43)cc21 ; c1ccc(-c2ccc(N(c3ccc(-c4cccc5c4oc4ccccc45)cc3)c3ccc(-n4c5ccccc5c5ccccc54)cc3)cc2)cc1 ; c1ccc(-c2ccc(N(c3ccc(-c4ccc(-c5cccc6ccccc56)cc4)cc3)c3ccc(-c4cccc5c4oc4ccccc45)cc3)cc2)cc1 ; c1ccc(N(c2ccc(-c3ccc(-c4cccc5ccccc45)cc3)cc2)c2ccc(-c3cccc4c3oc3ccccc34)cc2)cc1]

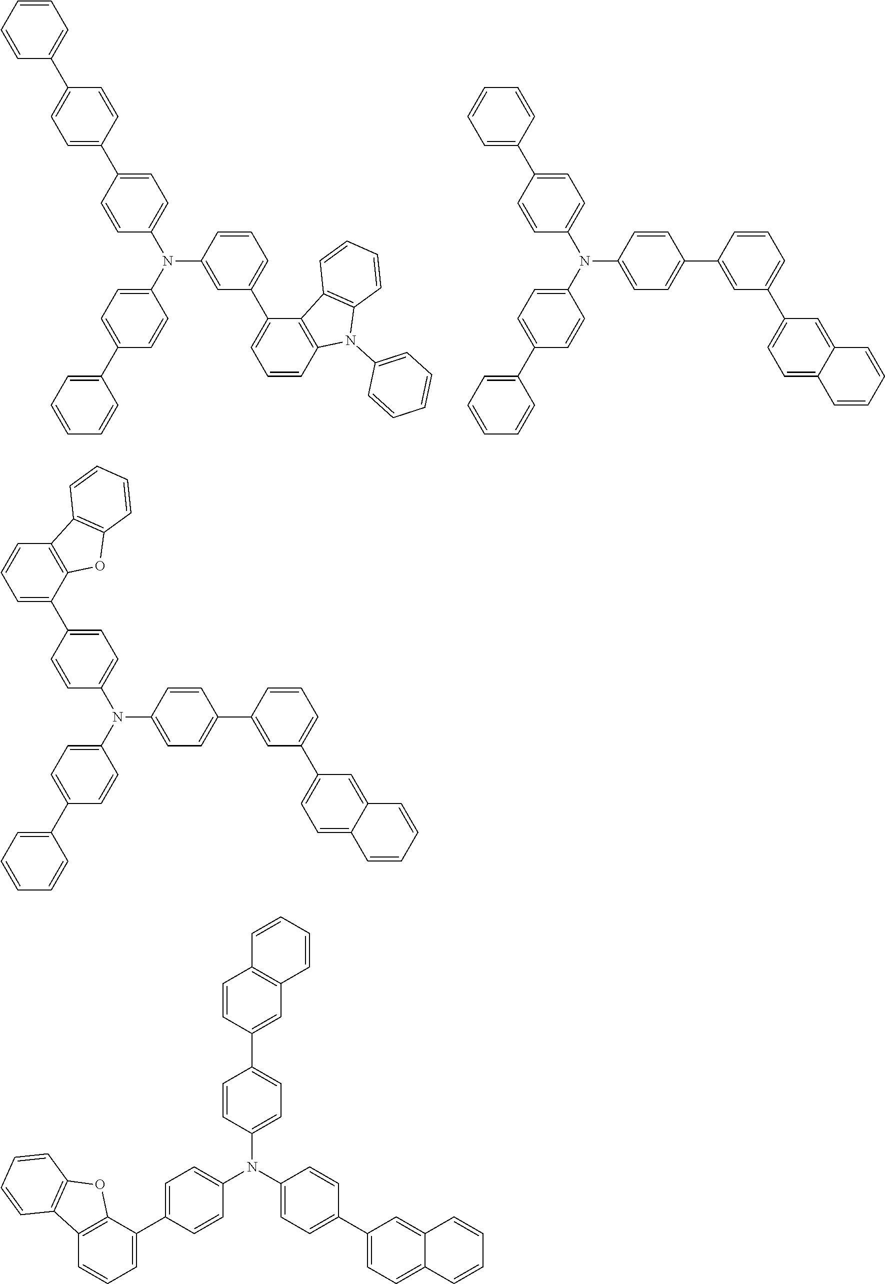

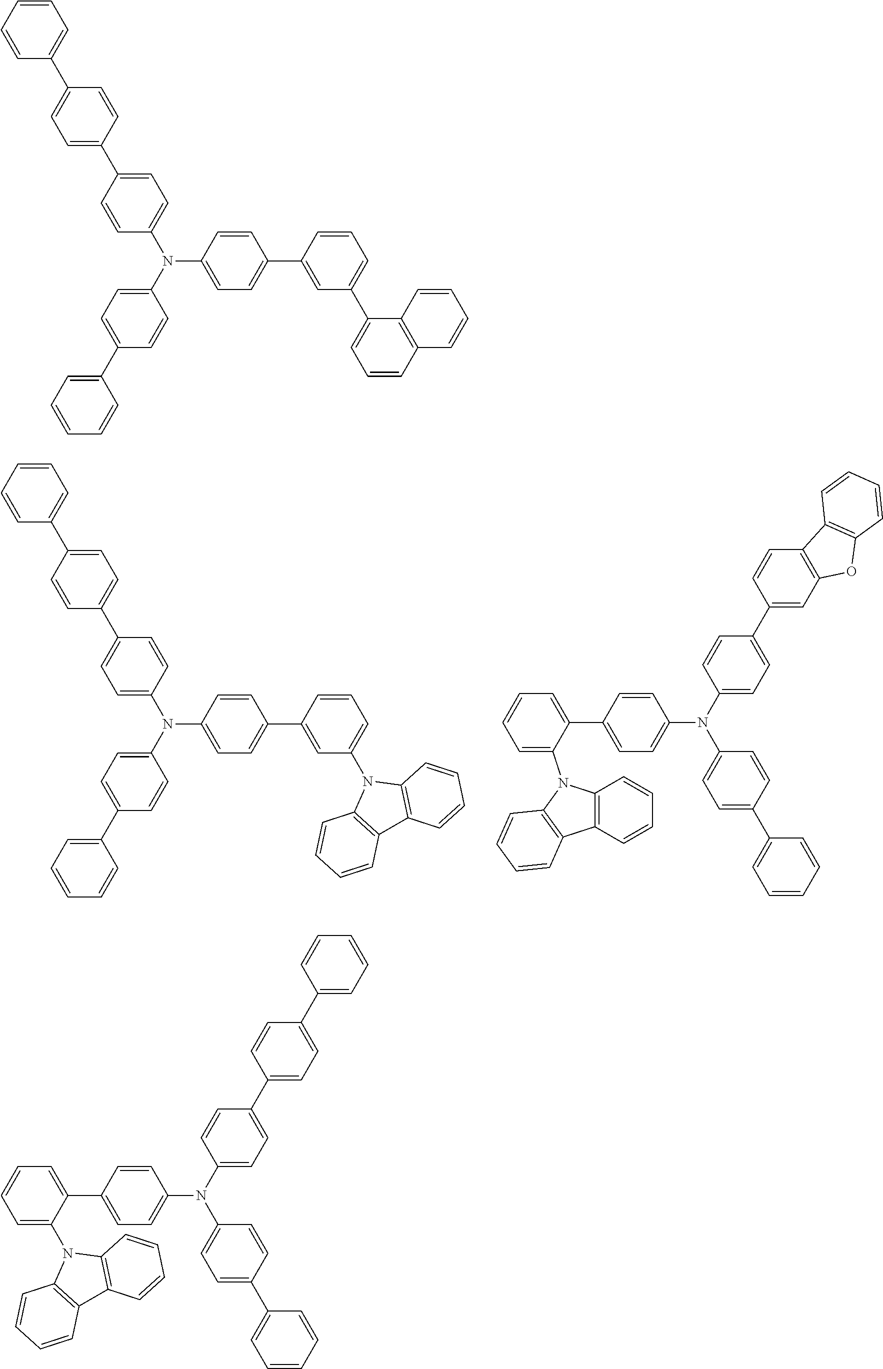

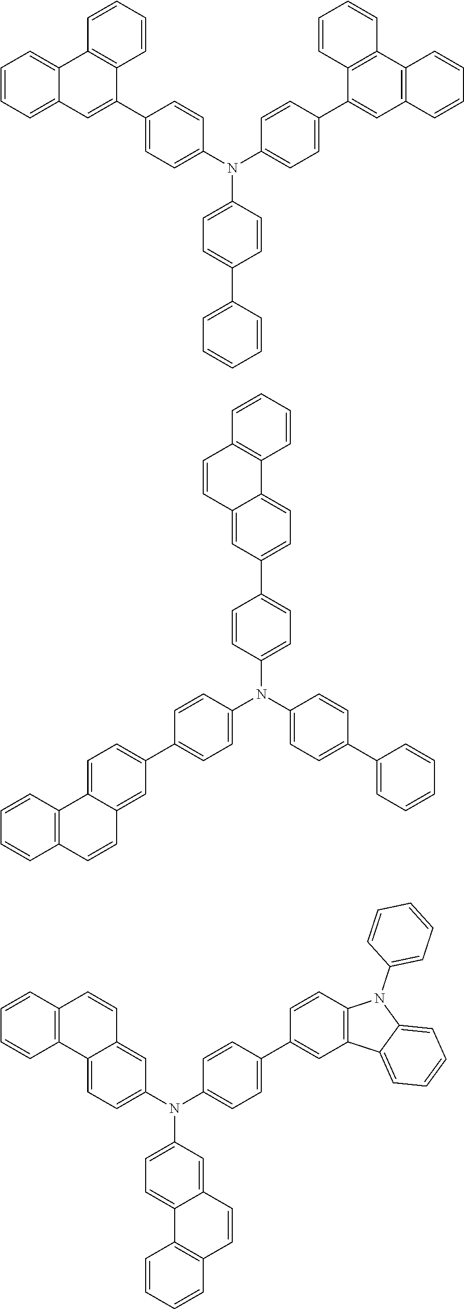

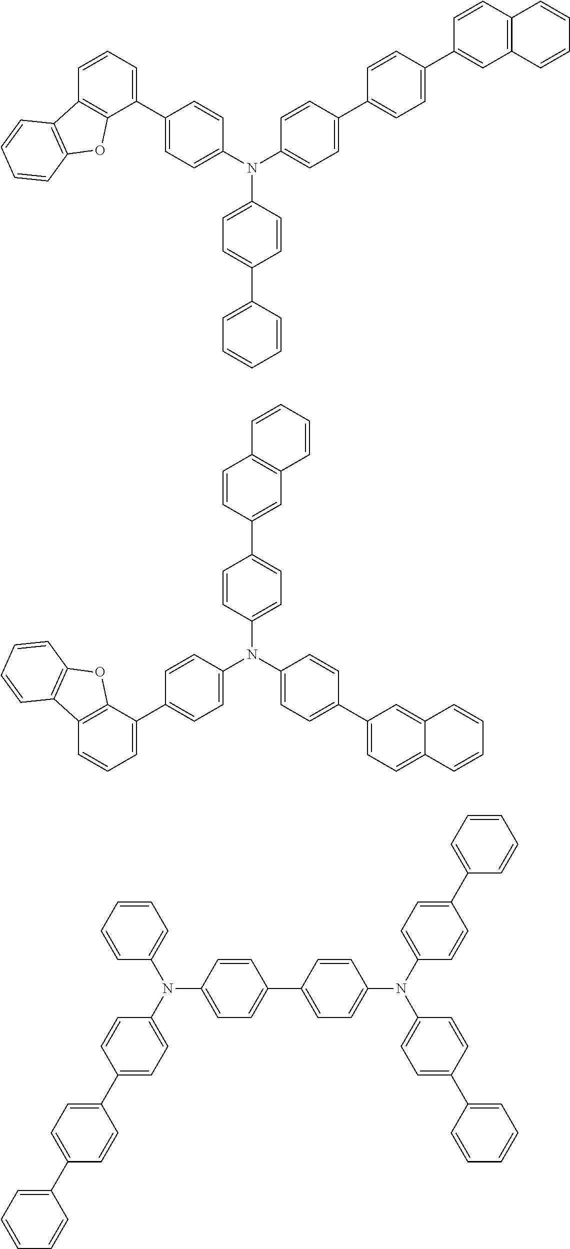

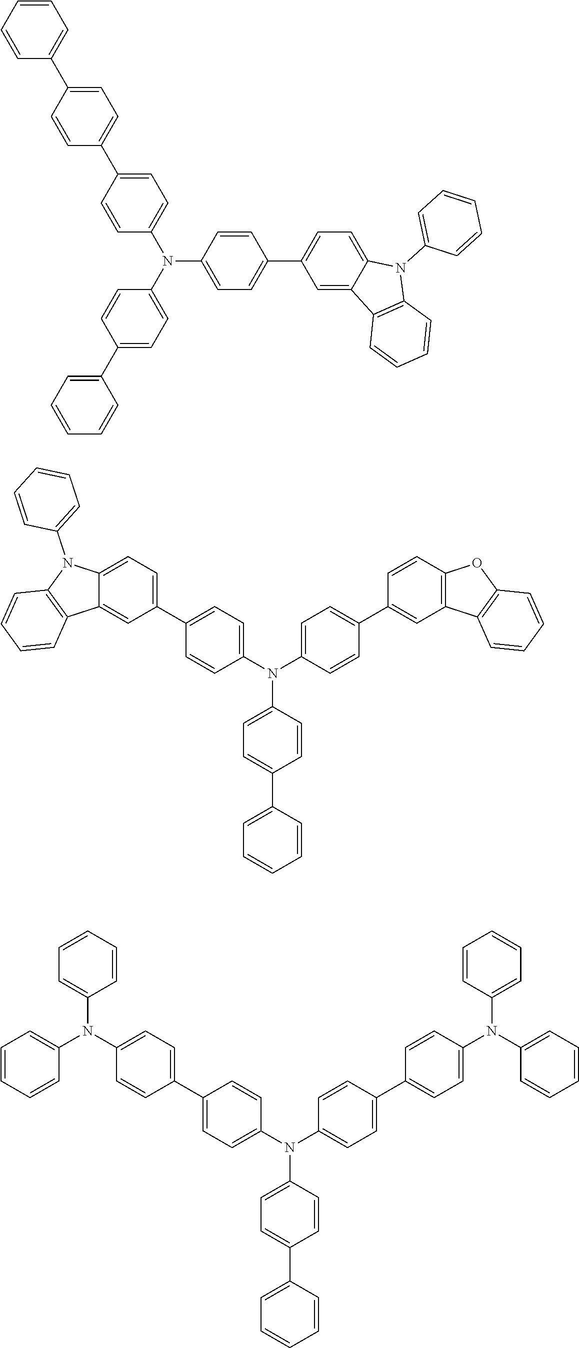

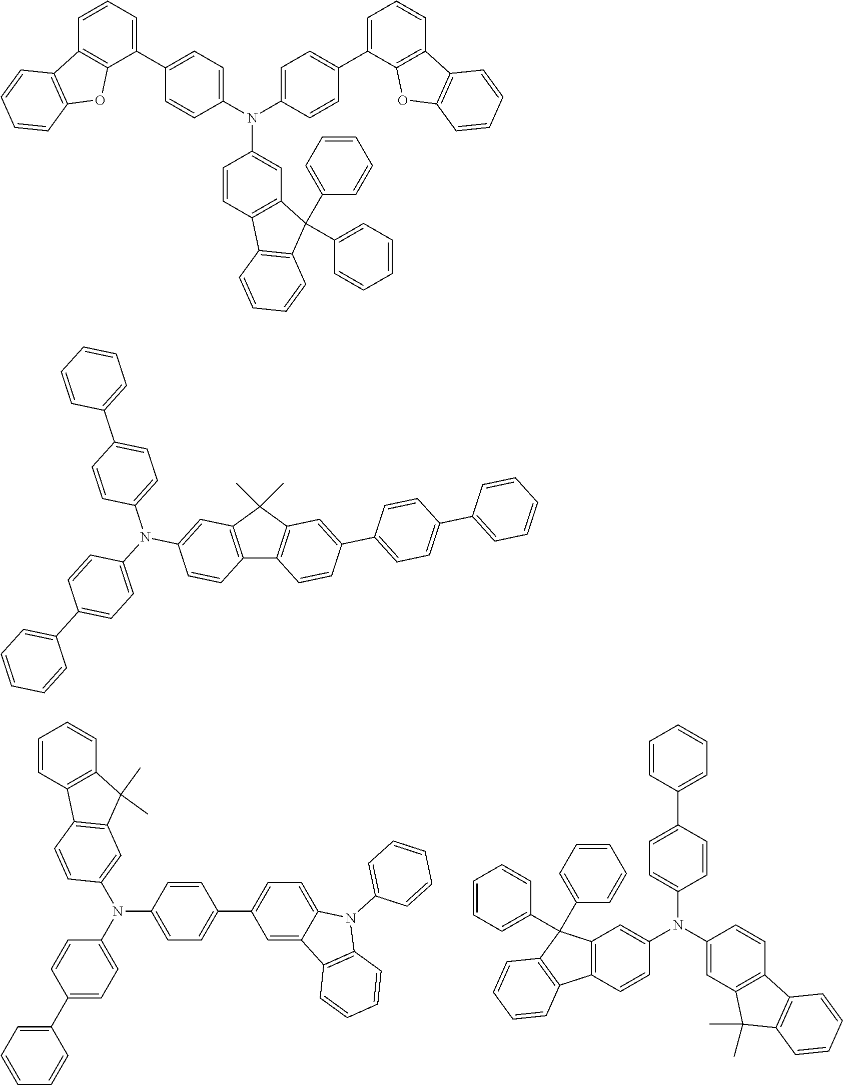

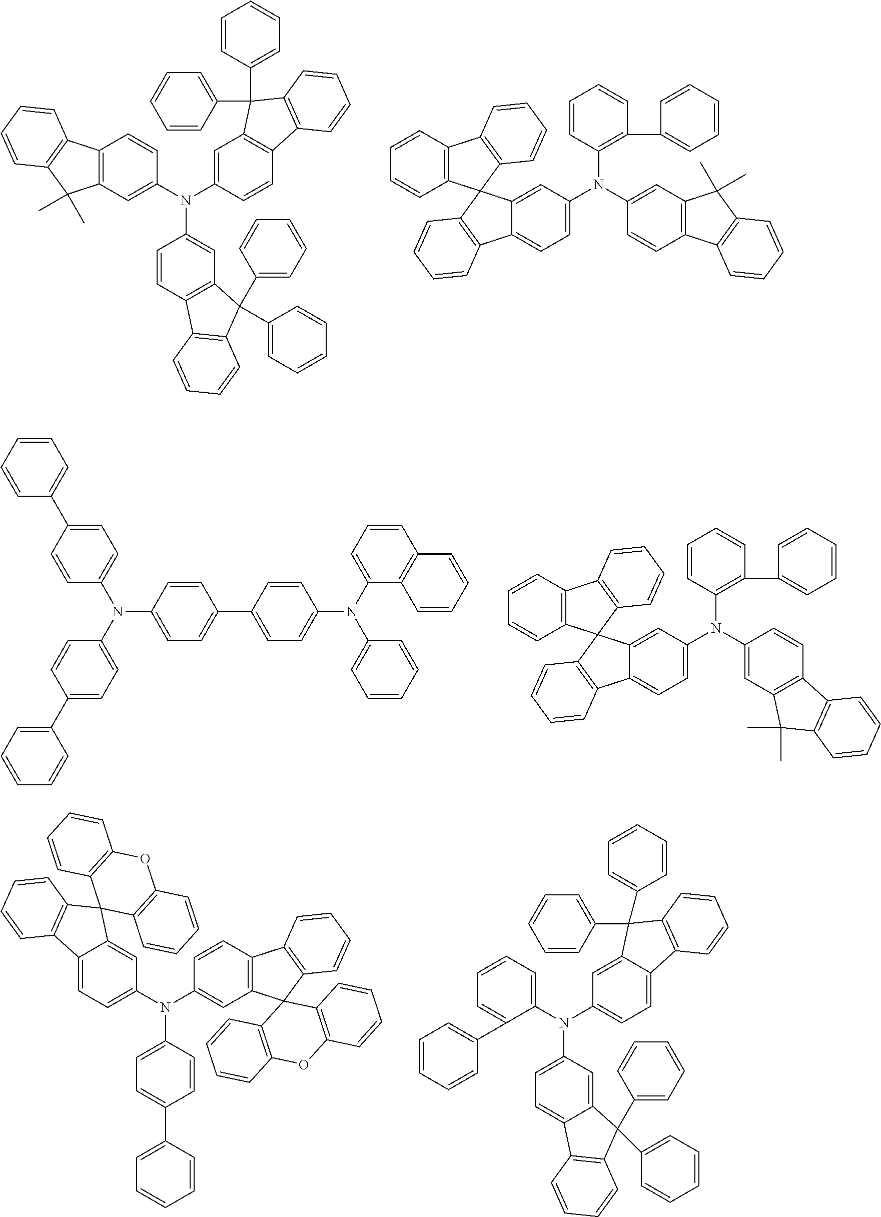

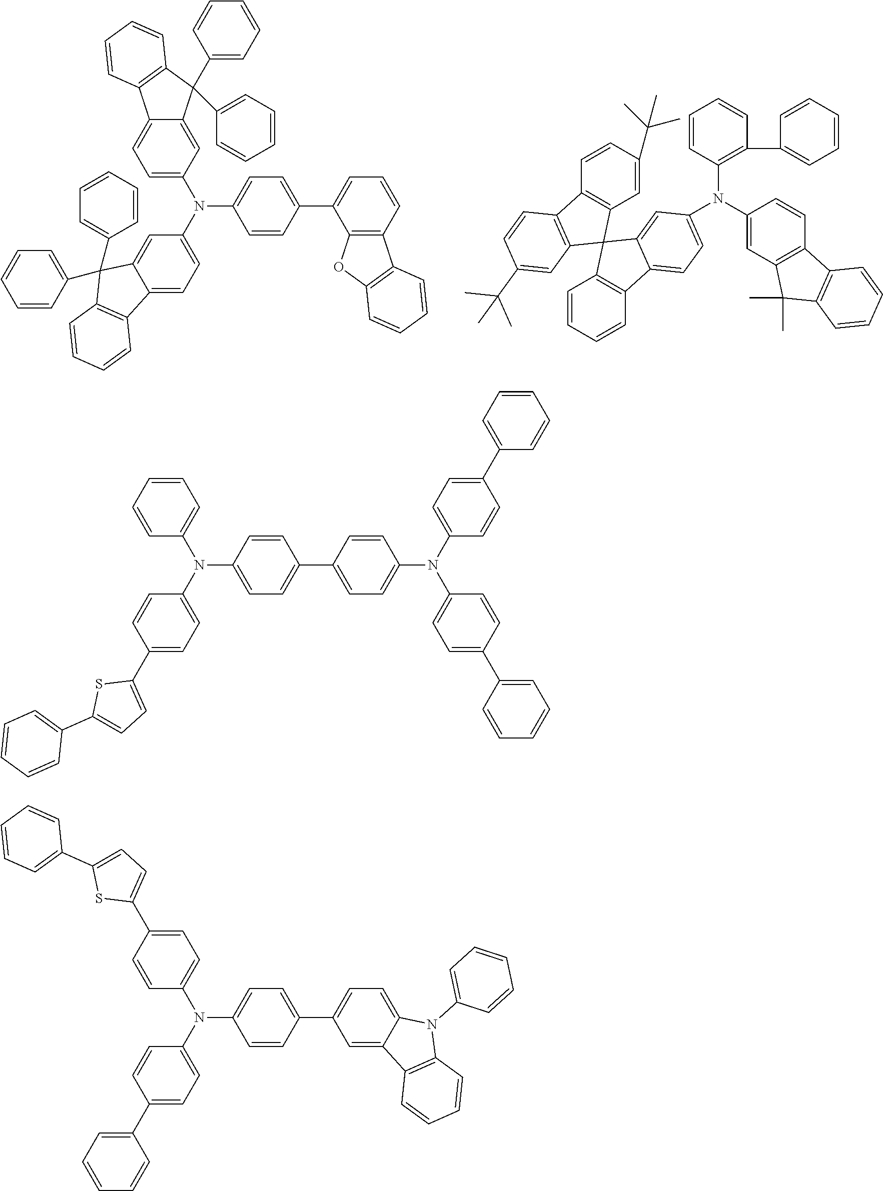

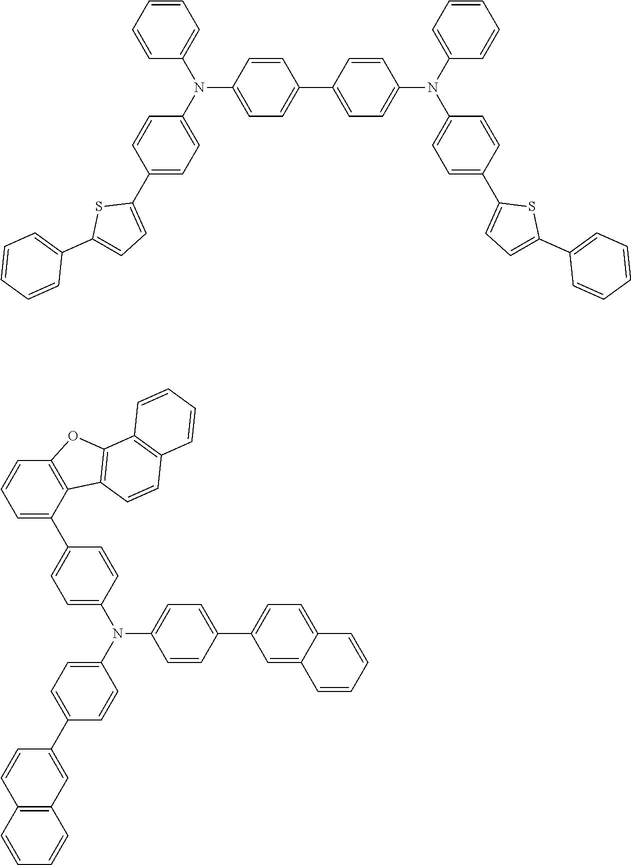 [SMILES: c1ccc(-c2ccc(-c3ccc(N(c4ccccc4)c4ccc(-c5ccc(N(c6ccccc6)c6ccc(-c7ccc(-c8ccccc8)s7)cc6)cc5)cc4)cc3)s2)cc1 ; c1ccc2cc(-c3ccc(N(c4ccc(-c5ccc6ccccc6c5)cc4)c4ccc(-c5cccc6oc7c8ccccc8ccc7c56)cc4)cc3)ccc2c1]

### Electron Blocking Layer

The electron blocking layer is preferably a layer for transporting holes and blocking electrons from reaching a layer close to the anode (e.g., the hole transporting layer) beyond the electron blocking layer.

A compound contained in the electron blocking layer is exemplified by a compound used in a known electron blocking layer and is preferably at least one compound selected from the group consisting of an aromatic amine compound and a carbazole derivative.

The compound contained in the electron blocking layer may be a monoamine compound having only one substituted or unsubstituted amino group in a molecule.

The compound contained in the electron blocking layer may be a compound having a substituted or unsubstituted carbazole group and one substituted or unsubstituted amino group in a molecule.

The electron blocking layer may block excitons generated in the emitting layer from being transferred to a layer(s) (e.g., the hole transporting layer and the hole injecting layer) close to the anode beyond the electron blocking layer in order that excited energy does not leak to the neighboring layers.

### Hole Blocking Layer

The hole blocking layer is preferably a layer for transporting electrons and blocking holes from reaching a layer close to the cathode (e.g., the electron transporting layer) beyond the hole blocking layer.

The compound contained in the hole blocking layer is exemplified by a compound used in a known hole blocking layer.

The compound contained in the electron hole blocking layer, which is exemplified by similar to a compound used usable in a known electron blocking transporting layer described later, and is preferably at least one compound selected from the group consisting of a metal complex, a heteroaromatic compound, and a high polymer compound.

The compound contained in the hole blocking layer may be, for instance, at least one compound selected from the group consisting of an imidazole derivative, benzimidazole derivative, azine derivative, carbazole derivative, and phenanthroline derivative.

In order to prevent excitation energy from leaking out from the emitting layer toward neighboring layer(s), the hole blocking layer is also preferably a layer blocking excitons generated in the emitting layer from being transferred to a layer(s) closer to the cathode (e.g., the electron transporting layer and the electron injecting layer) beyond the hole blocking layer.

### Electron Transporting Layer

The electron transporting layer is a layer containing a highly electron-transporting substance.

In the organic EL device according to the third exemplary embodiment, the electron transporting layer preferably contains a fourth compound.

In the organic EL device according to the third exemplary embodiment, the electron transporting layer is preferably provided between the emitting zone and the cathode.

In the organic EL device according to the third exemplary embodiment, the electron transporting layer preferably contains the fourth compound represented by a formula (E1) below.

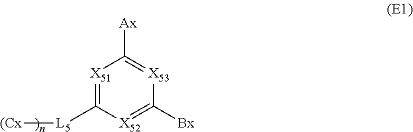

In the formula (E1):
X₅₁, X₅₂, and X₅₃ are each independently a nitrogen atom or CR₅;
at least one of X₅₁, X₅₂, or X₅₃ is a nitrogen atom;
R₅ is a hydrogen atom, a cyano group, a substituted or unsubstituted alkyl group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a substituted or unsubstituted aryl group having 6 to 50 ring carbon atoms, or a substituted or unsubstituted heterocyclic group having 5 to 50 ring atoms;
Ax is a substituted or unsubstituted aryl group having 6 to 18 ring carbon atoms, or a substituted or unsubstituted heterocyclic group having 5 to 13 ring atoms;
Bx is a substituted or unsubstituted aryl group having 6 to 18 ring carbon atoms, or a substituted or unsubstituted heterocyclic group having 5 to 13 ring atoms;
L₅ is a single bond, a substituted or unsubstituted (n+1)-valent aromatic hydrocarbon ring group having 6 to 18 ring carbon atoms, a substituted or unsubstituted (n+1)-valent heterocyclic group having 5 to 13 ring atoms, or an (n+1)-valent group derived from bonding of two or three selected from the group consisting of substituted or unsubstituted aromatic hydrocarbon ring groups having 6 to 18 ring carbon atoms and substituted or unsubstituted heterocyclic groups having 5 to 13 ring atoms;
n is 1, 2, or 3, and when n is 2 or 3, L₅ is not a single bond;
each Cx independently represents a substituted or unsubstituted aryl group having 6 to 30 ring carbon atoms, or a substituted or unsubstituted heterocyclic group having 5 to 60 ring atoms; and
when a plurality of Cx are present, the plurality of Cx are mutually the same or different.

In the fourth compound, R₉₀₁, R₉₀₂, R₉₀₃, and R₉₀₄ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different; and
when a plurality of R₉₀₄ are present, the plurality of R₉₀₄ are mutually the same or different.

In the organic EL device according to the third exemplary embodiment, two or three of X₅₁, X₅₂, and X₅₃ in the fourth compound are preferably nitrogen atoms.

In the organic EL device according to the third exemplary embodiment, the fourth compound is preferably a compound represented by a formula (E11), (E12), (E13), or (E14) below.

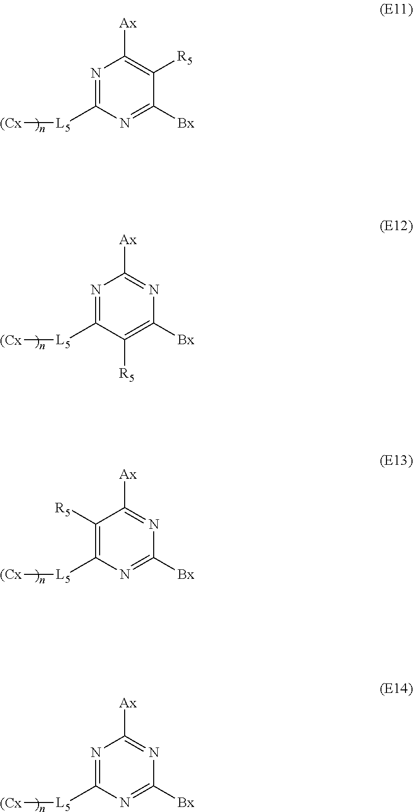

In the formulae (E11) to (E14), Ax, Bx, Cx, R₅, L₅, and n are as defined in the formula (E1).

In the organic EL device according to the third exemplary embodiment, the substituent for the substituted or unsubstituted group is also preferably a group selected from the group consisting of an alkyl group having 1 to 50 carbon atoms, an aryl group having 6 to 50 ring carbon atoms, and a heterocyclic group having 5 to 50 ring atoms.

In the organic EL device according to the third exemplary embodiment, the substituent for the substituted or unsubstituted group is also preferably a group selected from the group consisting of an alkyl group having 1 to 18 carbon atoms, an aryl group having 6 to 18 ring carbon atoms, and a heterocyclic group having 5 to 18 ring atoms.

For the electron transporting layer in the organic EL device according to the third exemplary embodiment, 1) metal complexes such as an aluminum complex, beryllium complex, and zinc complex, 2) hetero aromatic compounds such as an imidazole derivative, benzimidazole derivative, azine derivative, carbazole derivative, and phenanthroline derivative, and 3) high polymer compounds are usable.

Specific examples of usable low-molecular organic compounds include metal complexes such as Alq, tris(4-methyl-8-quinolinolato)aluminum (abbreviation: Almq₃), bis(10-hydroxybenzo[h]quinolinato)beryllium (abbreviation: BeBq₂), BAlq, Znq, ZnPBO, and ZnBTZ.

In addition to the metal complex, a heteroaromatic compound such as 2-(4-biphenylyl)-5-(4-tert-butylphenyl)-1,3,4-oxadiazole (abbreviation: PBD), 1,3-bis[5-(ptert-butylphenyl)-1,3,4-oxadiazole-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazole-2-yl)stilbene (abbreviation: BzOs) is usable.

In the exemplary embodiment, a benzimidazole compound is preferably usable.

The above-described substances mostly have an electron mobility of 10⁶ cm²/(V·s) or more.

It should be noted that any substance other than the above substance may be used for the electron transporting layer as long as the substance exhibits a higher electron transportability than the hole transportability.

The electron transporting layer may be provided in the form of a single layer or a laminate of two or more layers of the above substance(s).

Further, a high polymer compound is usable for the electron transporting layer.

For instance, poly[(9,9-dihexylfluorene-2,7-diyl)-co-(pyridine-3,5-diyl)](abbreviation: PF-Py), poly[(9,9-dioctylfluorene-2,7-diyl)-co-(2,2′-bipyridine-6,6′-diyl)] (abbreviation: PF-BPy) and the like are usable.

### Specific Examples of Fourth Compound

Specific examples of the fourth compound include the following compounds.

However, the invention is not limited to these specific examples of the fourth compound.

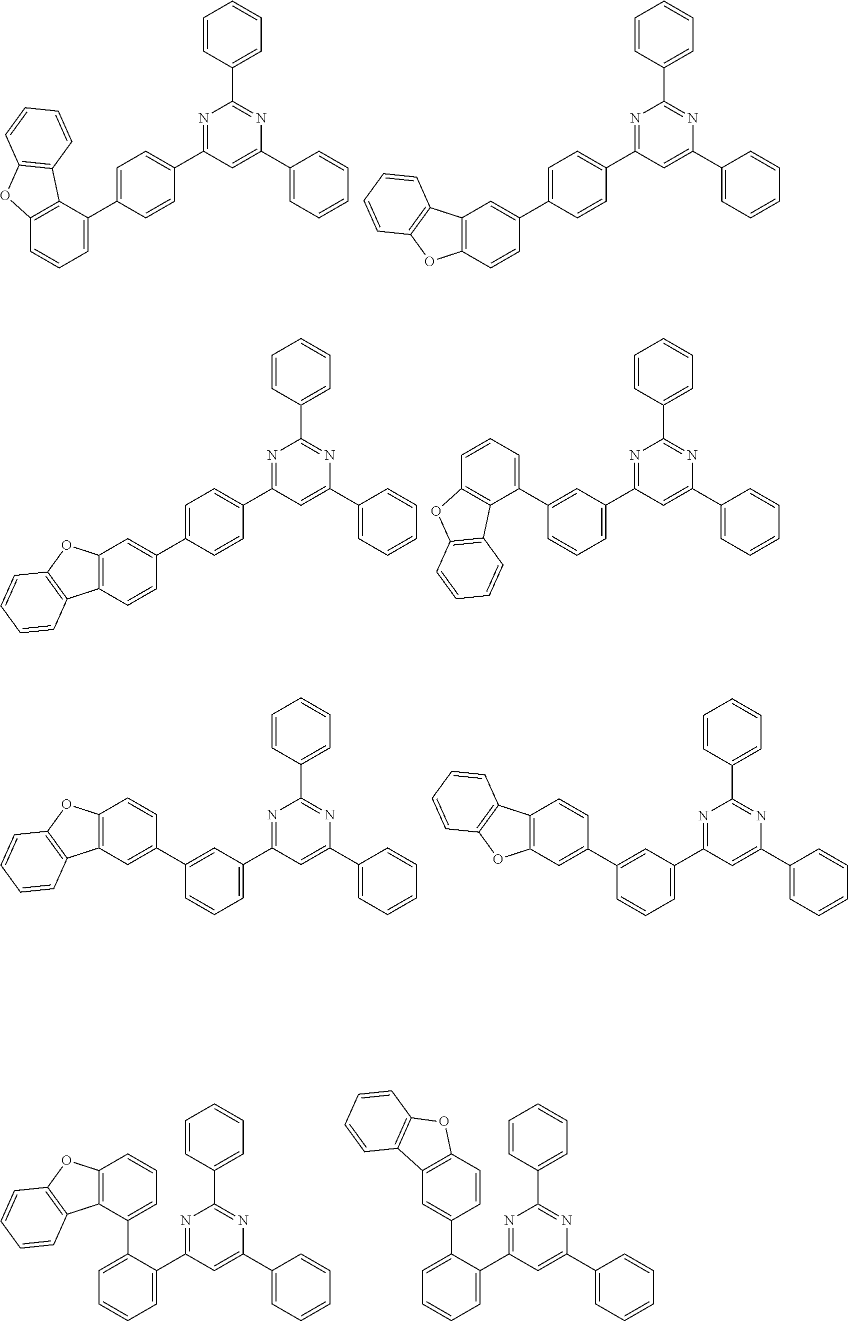

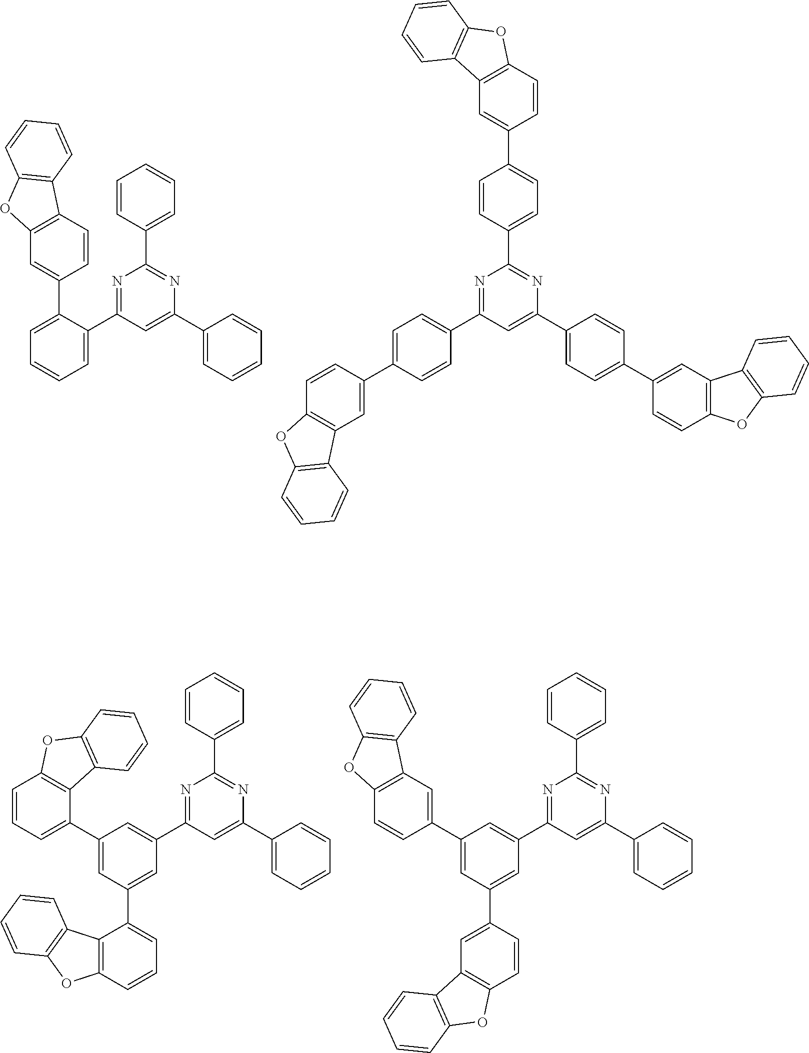 [SMILES: c1ccc(-c2cc(-c3ccccc3-c3ccc4c(c3)oc3ccccc34)nc(-c3ccccc3)n2)cc1 ; c1ccc2c(c1)oc1ccc(-c3ccc(-c4cc(-c5ccc(-c6ccc7oc8ccccc8c7c6)cc5)nc(-c5ccc(-c6ccc7oc8ccccc8c7c6)cc5)n4)cc3)cc12 ; c1ccc(-c2cc(-c3cc(-c4cccc5oc6ccccc6c45)cc(-c4cccc5oc6ccccc6c45)c3)nc(-c3ccccc3)n2)cc1 ; c1ccc(-c2cc(-c3cc(-c4ccc5oc6ccccc6c5c4)cc(-c4ccc5oc6ccccc6c5c4)c3)nc(-c3ccccc3)n2)cc1]

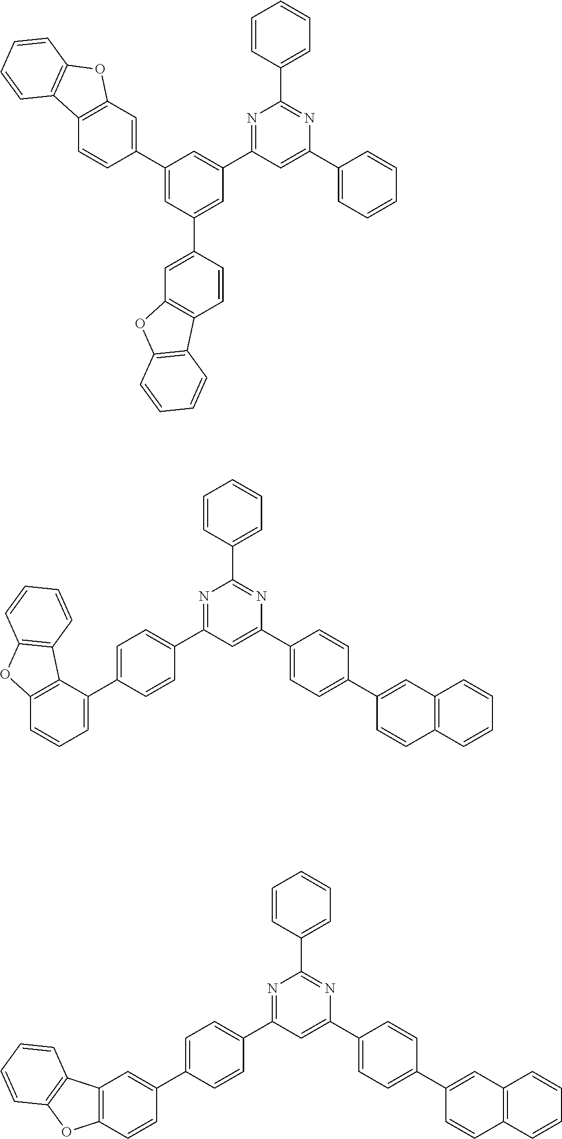 [SMILES: c1ccc(-c2cc(-c3cc(-c4ccc5c(c4)oc4ccccc45)cc(-c4ccc5c(c4)oc4ccccc45)c3)nc(-c3ccccc3)n2)cc1 ; c1ccc(-c2nc(-c3ccc(-c4ccc5ccccc5c4)cc3)cc(-c3ccc(-c4cccc5oc6ccccc6c45)cc3)n2)cc1 ; c1ccc(-c2nc(-c3ccc(-c4ccc5ccccc5c4)cc3)cc(-c3ccc(-c4ccc5oc6ccccc6c5c4)cc3)n2)cc1]

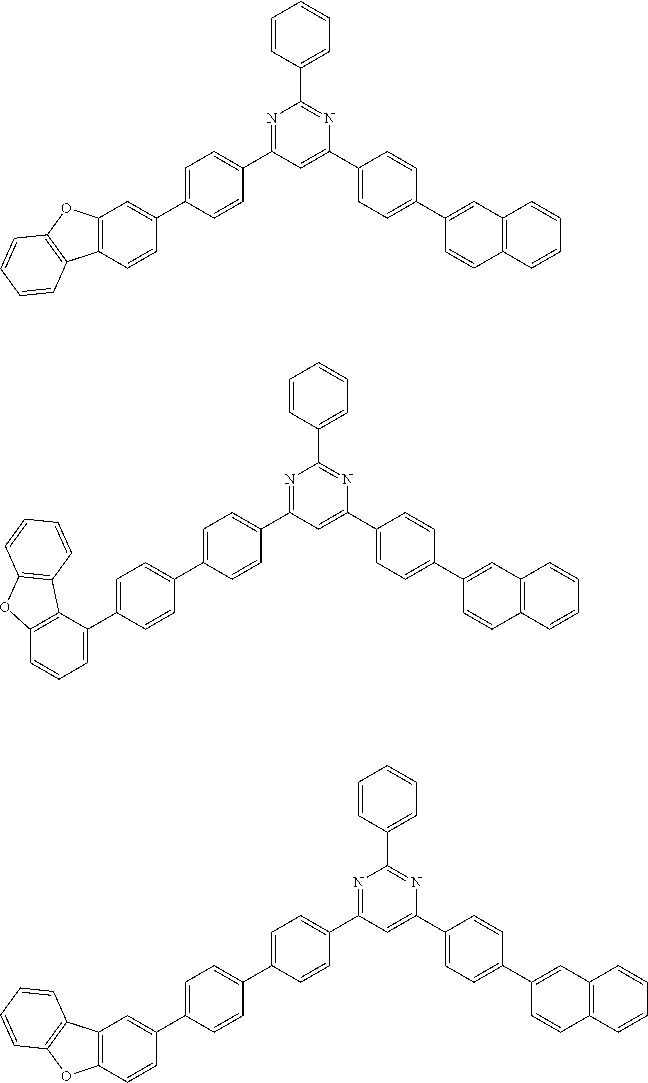 [SMILES: c1ccc(-c2nc(-c3ccc(-c4ccc5ccccc5c4)cc3)cc(-c3ccc(-c4ccc5c(c4)oc4ccccc45)cc3)n2)cc1 ; c1ccc(-c2nc(-c3ccc(-c4ccc(-c5cccc6oc7ccccc7c56)cc4)cc3)cc(-c3ccc(-c4ccc5ccccc5c4)cc3)n2)cc1 ; c1ccc(-c2nc(-c3ccc(-c4ccc(-c5ccc6oc7ccccc7c6c5)cc4)cc3)cc(-c3ccc(-c4ccc5ccccc5c4)cc3)n2)cc1]

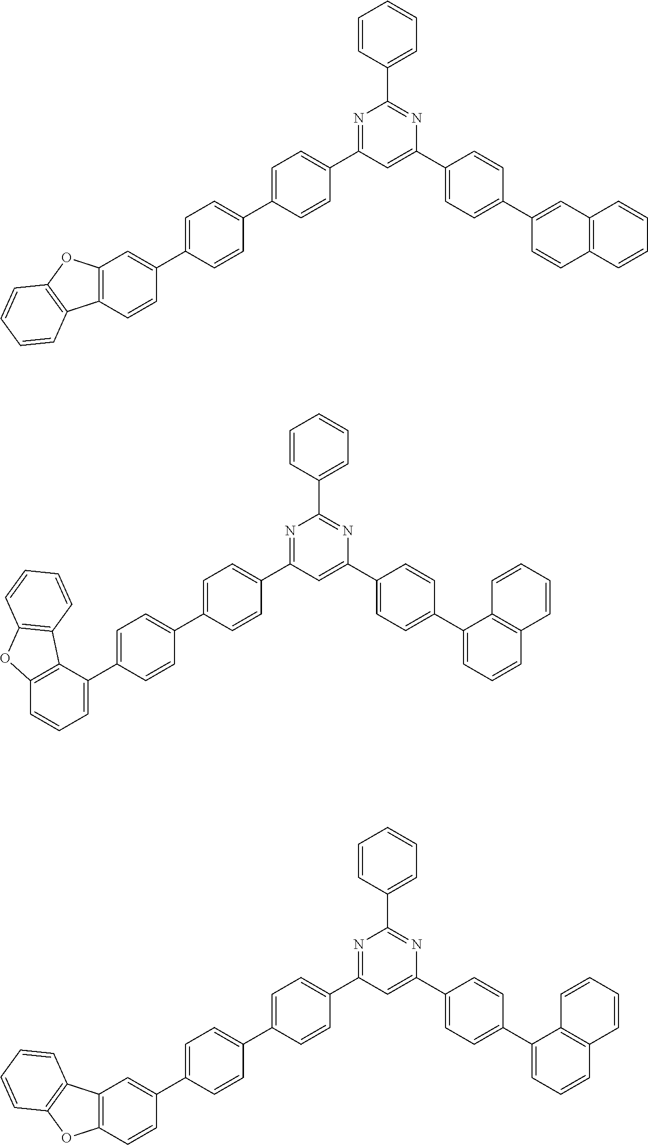 [SMILES: c1ccc(-c2nc(-c3ccc(-c4ccc(-c5ccc6c(c5)oc5ccccc56)cc4)cc3)cc(-c3ccc(-c4ccc5ccccc5c4)cc3)n2)cc1 ; c1ccc(-c2nc(-c3ccc(-c4ccc(-c5cccc6oc7ccccc7c56)cc4)cc3)cc(-c3ccc(-c4cccc5ccccc45)cc3)n2)cc1 ; c1ccc(-c2nc(-c3ccc(-c4ccc(-c5ccc6oc7ccccc7c6c5)cc4)cc3)cc(-c3ccc(-c4cccc5ccccc45)cc3)n2)cc1]

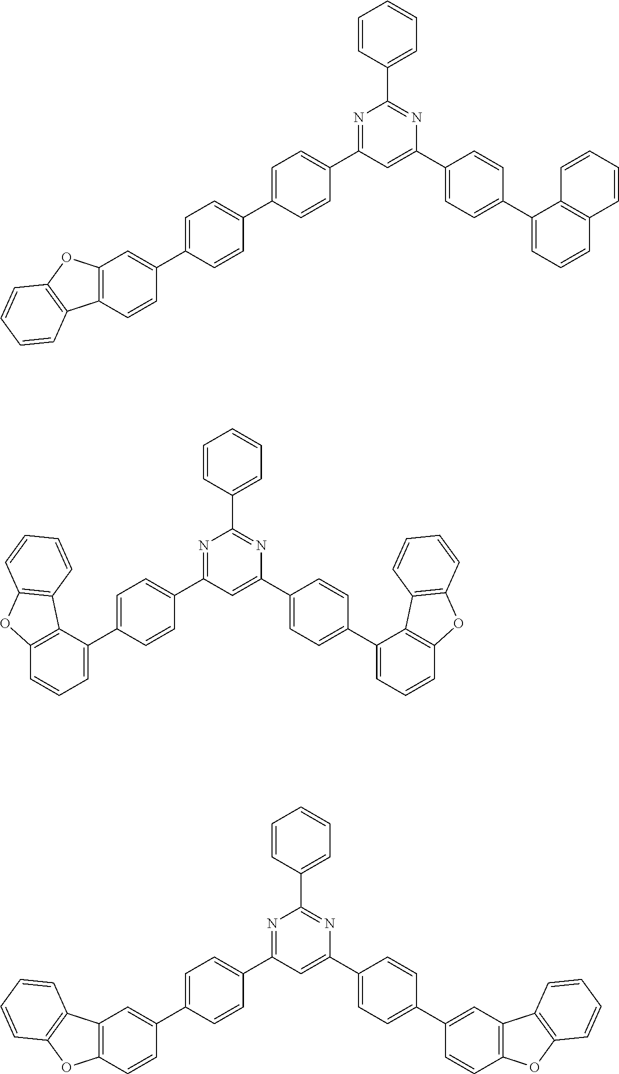 [SMILES: c1ccc(-c2nc(-c3ccc(-c4ccc(-c5ccc6c(c5)oc5ccccc56)cc4)cc3)cc(-c3ccc(-c4cccc5ccccc45)cc3)n2)cc1 ; c1ccc(-c2nc(-c3ccc(-c4cccc5oc6ccccc6c45)cc3)cc(-c3ccc(-c4cccc5oc6ccccc6c45)cc3)n2)cc1 ; c1ccc(-c2nc(-c3ccc(-c4ccc5oc6ccccc6c5c4)cc3)cc(-c3ccc(-c4ccc5oc6ccccc6c5c4)cc3)n2)cc1]

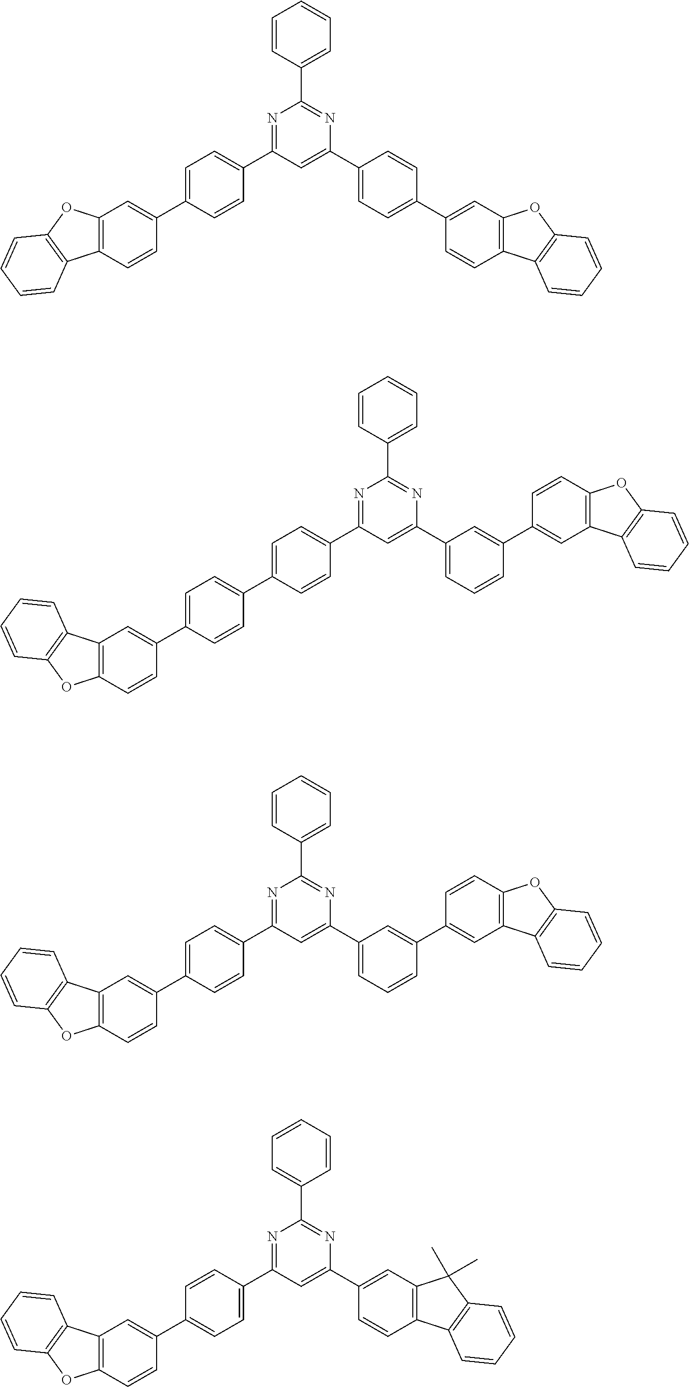 [SMILES: c1ccc(-c2nc(-c3ccc(-c4ccc5c(c4)oc4ccccc45)cc3)cc(-c3ccc(-c4ccc5c(c4)oc4ccccc45)cc3)n2)cc1 ; c1ccc(-c2nc(-c3ccc(-c4ccc(-c5ccc6oc7ccccc7c6c5)cc4)cc3)cc(-c3cccc(-c4ccc5oc6ccccc6c5c4)c3)n2)cc1 ; c1ccc(-c2nc(-c3ccc(-c4ccc5oc6ccccc6c5c4)cc3)cc(-c3cccc(-c4ccc5oc6ccccc6c5c4)c3)n2)cc1 ; CC1(C)c2ccccc2-c2ccc(-c3cc(-c4ccc(-c5ccc6oc7ccccc7c6c5)cc4)nc(-c4ccccc4)n3)cc21]

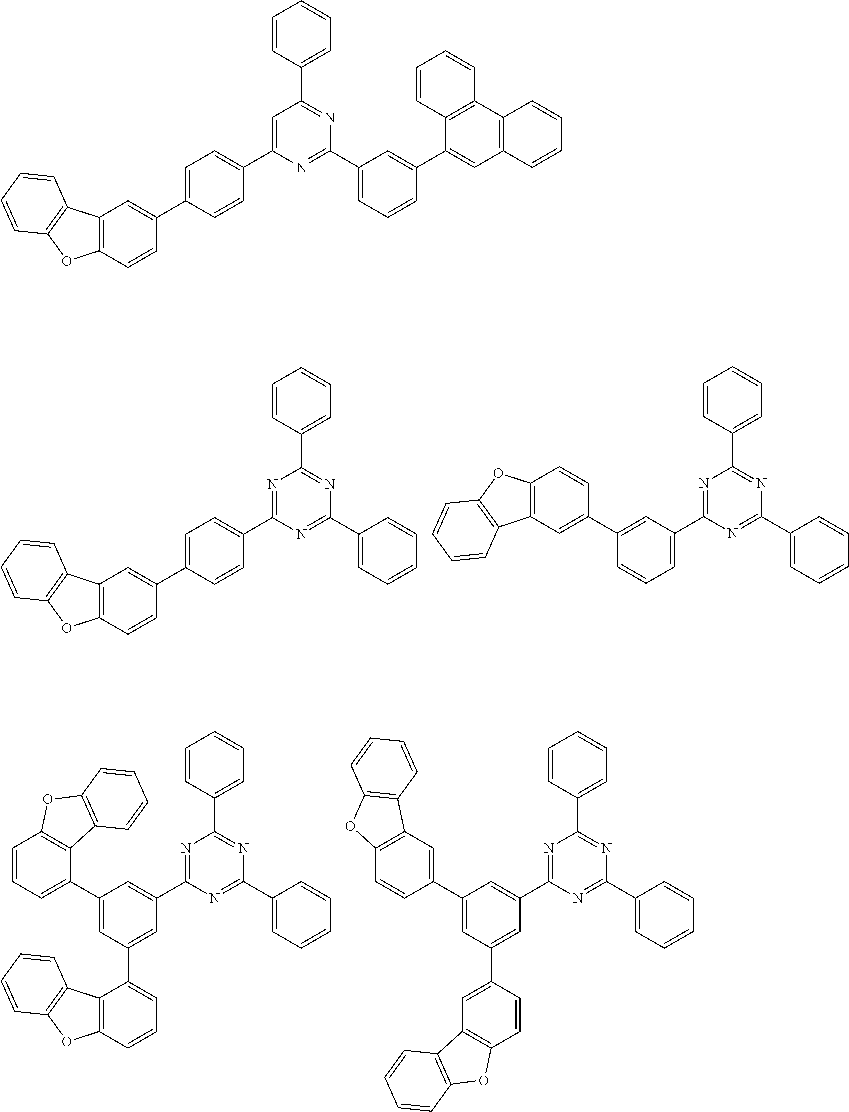

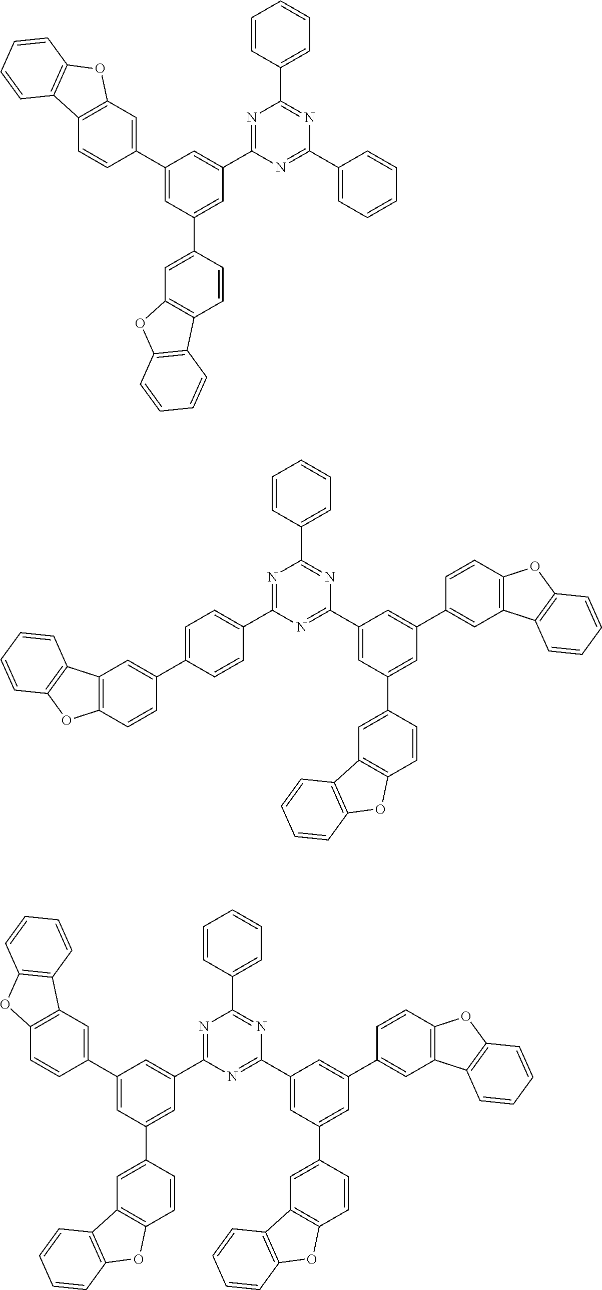 [SMILES: c1ccc(-c2nc(-c3ccccc3)nc(-c3cc(-c4ccc5c(c4)oc4ccccc45)cc(-c4ccc5c(c4)oc4ccccc45)c3)n2)cc1 ; c1ccc(-c2nc(-c3ccc(-c4ccc5oc6ccccc6c5c4)cc3)nc(-c3cc(-c4ccc5oc6ccccc6c5c4)cc(-c4ccc5oc6ccccc6c5c4)c3)n2)cc1 ; c1ccc(-c2nc(-c3cc(-c4ccc5oc6ccccc6c5c4)cc(-c4ccc5oc6ccccc6c5c4)c3)nc(-c3cc(-c4ccc5oc6ccccc6c5c4)cc(-c4ccc5oc6ccccc6c5c4)c3)n2)cc1]

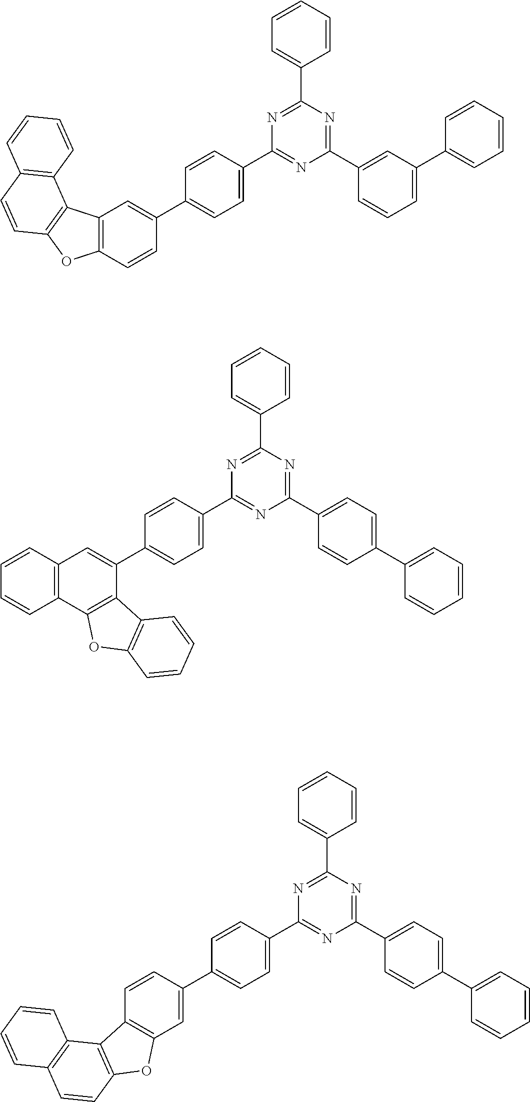 [SMILES: c1ccc(-c2cccc(-c3nc(-c4ccccc4)nc(-c4ccc(-c5ccc6oc7ccc8ccccc8c7c6c5)cc4)n3)c2)cc1 ; c1ccc(-c2ccc(-c3nc(-c4ccccc4)nc(-c4ccc(-c5cc6ccccc6c6oc7ccccc7c56)cc4)n3)cc2)cc1 ; c1ccc(-c2ccc(-c3nc(-c4ccccc4)nc(-c4ccc(-c5ccc6c(c5)oc5ccc7ccccc7c56)cc4)n3)cc2)cc1]

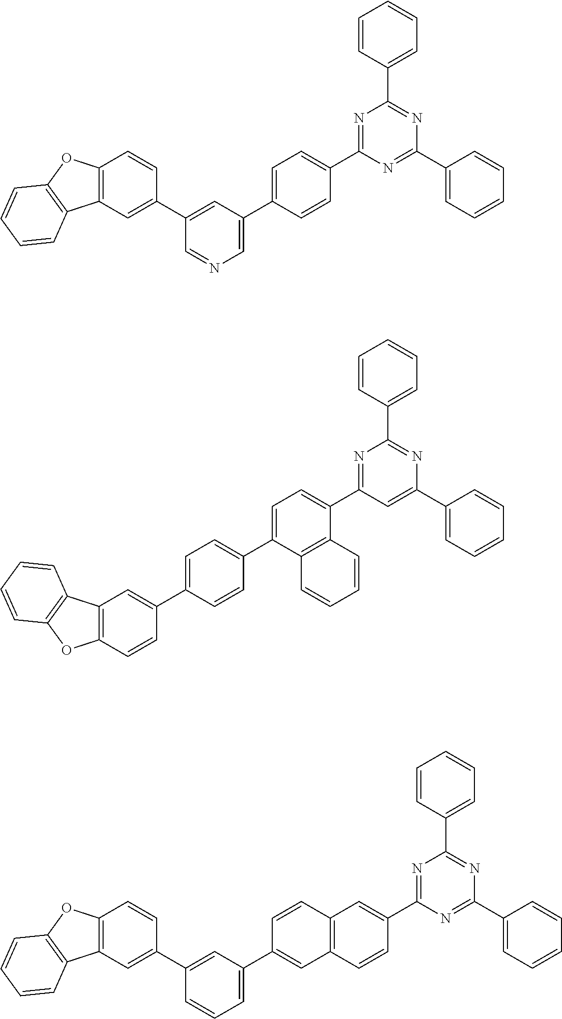 [SMILES: c1ccc(-c2nc(-c3ccccc3)nc(-c3ccc(-c4cncc(-c5ccc6oc7ccccc7c6c5)c4)cc3)n2)cc1 ; c1ccc(-c2cc(-c3ccc(-c4ccc(-c5ccc6oc7ccccc7c6c5)cc4)c4ccccc34)nc(-c3ccccc3)n2)cc1 ; c1ccc(-c2nc(-c3ccccc3)nc(-c3ccc4cc(-c5cccc(-c6ccc7oc8ccccc8c7c6)c5)ccc4c3)n2)cc1]

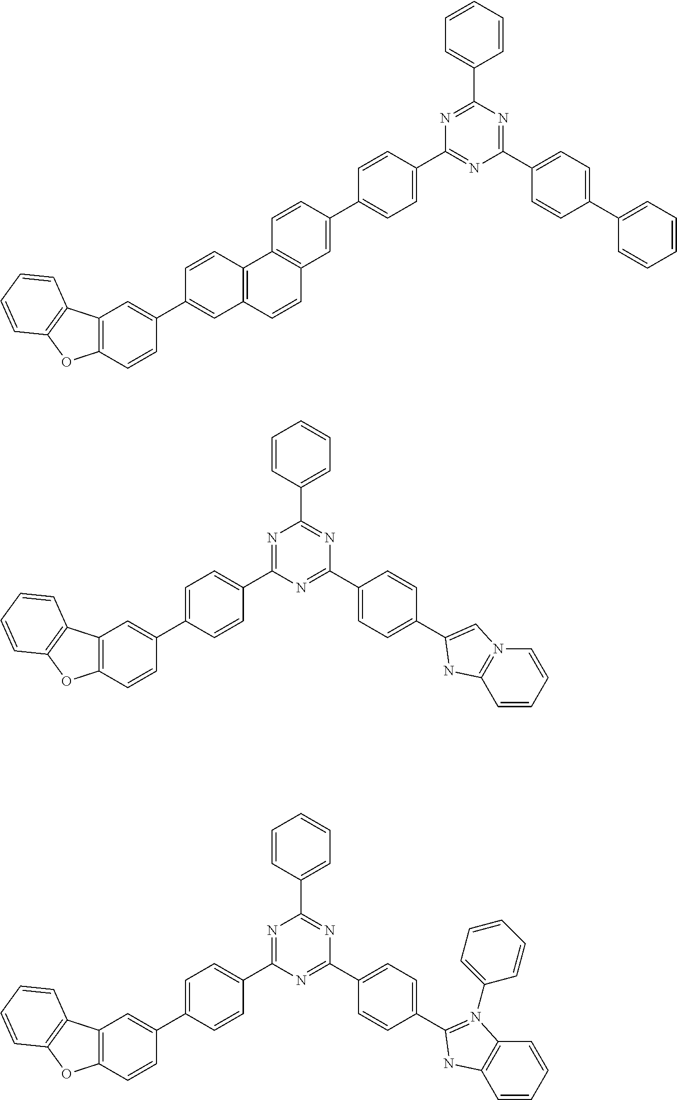

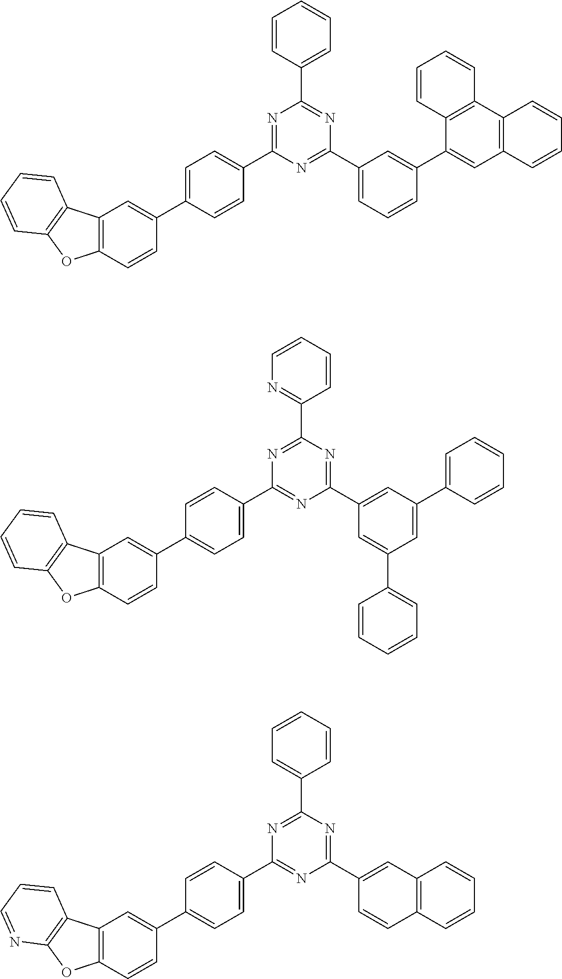 [SMILES: c1ccc(-c2nc(-c3ccc(-c4ccc5oc6ccccc6c5c4)cc3)nc(-c3cccc(-c4cc5ccccc5c5ccccc45)c3)n2)cc1 ; c1ccc(-c2cc(-c3ccccc3)cc(-c3nc(-c4ccc(-c5ccc6oc7ccccc7c6c5)cc4)nc(-c4ccccn4)n3)c2)cc1 ; c1ccc(-c2nc(-c3ccc(-c4ccc5oc6ncccc6c5c4)cc3)nc(-c3ccc4ccccc4c3)n2)cc1]

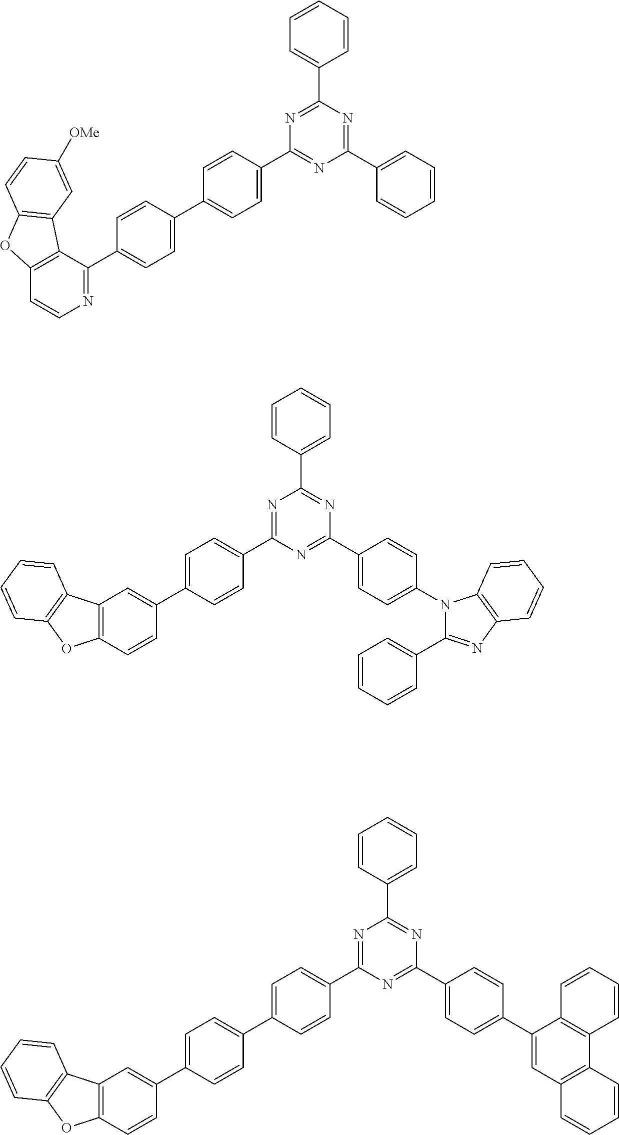 [SMILES: COc1ccc2oc3ccnc(-c4ccc(-c5ccc(-c6nc(-c7ccccc7)nc(-c7ccccc7)n6)cc5)cc4)c3c2c1 ; c1ccc(-c2nc(-c3ccc(-c4ccc5oc6ccccc6c5c4)cc3)nc(-c3ccc(-n4c(-c5ccccc5)nc5ccccc54)cc3)n2)cc1 ; c1ccc(-c2nc(-c3ccc(-c4ccc(-c5ccc6oc7ccccc7c6c5)cc4)cc3)nc(-c3ccc(-c4cc5ccccc5c5ccccc45)cc3)n2)cc1]

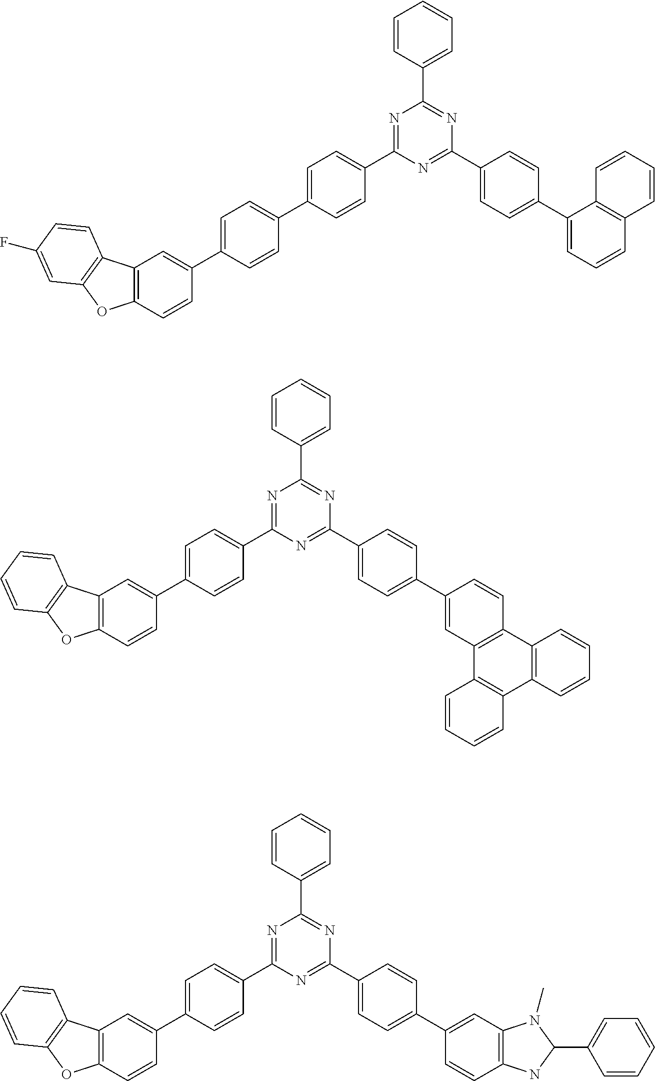 [SMILES: Fc1ccc2c(c1)oc1ccc(-c3ccc(-c4ccc(-c5nc(-c6ccccc6)nc(-c6ccc(-c7cccc8ccccc78)cc6)n5)cc4)cc3)cc12 ; c1ccc(-c2nc(-c3ccc(-c4ccc5oc6ccccc6c5c4)cc3)nc(-c3ccc(-c4ccc5c6ccccc6c6ccccc6c5c4)cc3)n2)cc1 ; CN1c2cc(-c3ccc(-c4nc(-c5ccccc5)nc(-c5ccc(-c6ccc7oc8ccccc8c7c6)cc5)n4)cc3)ccc2NC1c1ccccc1]

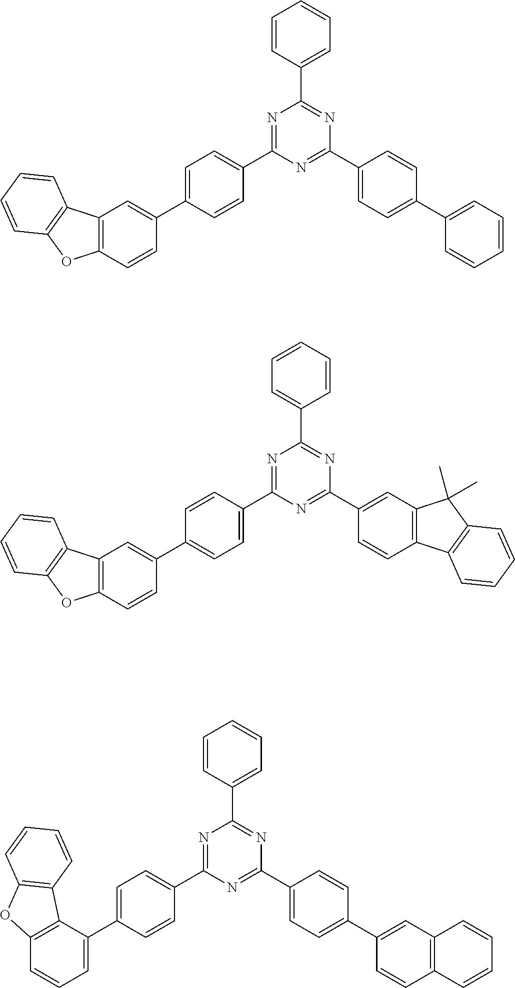 [SMILES: c1ccc(-c2ccc(-c3nc(-c4ccccc4)nc(-c4ccc(-c5ccc6oc7ccccc7c6c5)cc4)n3)cc2)cc1 ; CC1(C)c2ccccc2-c2ccc(-c3nc(-c4ccccc4)nc(-c4ccc(-c5ccc6oc7ccccc7c6c5)cc4)n3)cc21 ; c1ccc(-c2nc(-c3ccc(-c4ccc5ccccc5c4)cc3)nc(-c3ccc(-c4cccc5oc6ccccc6c45)cc3)n2)cc1]

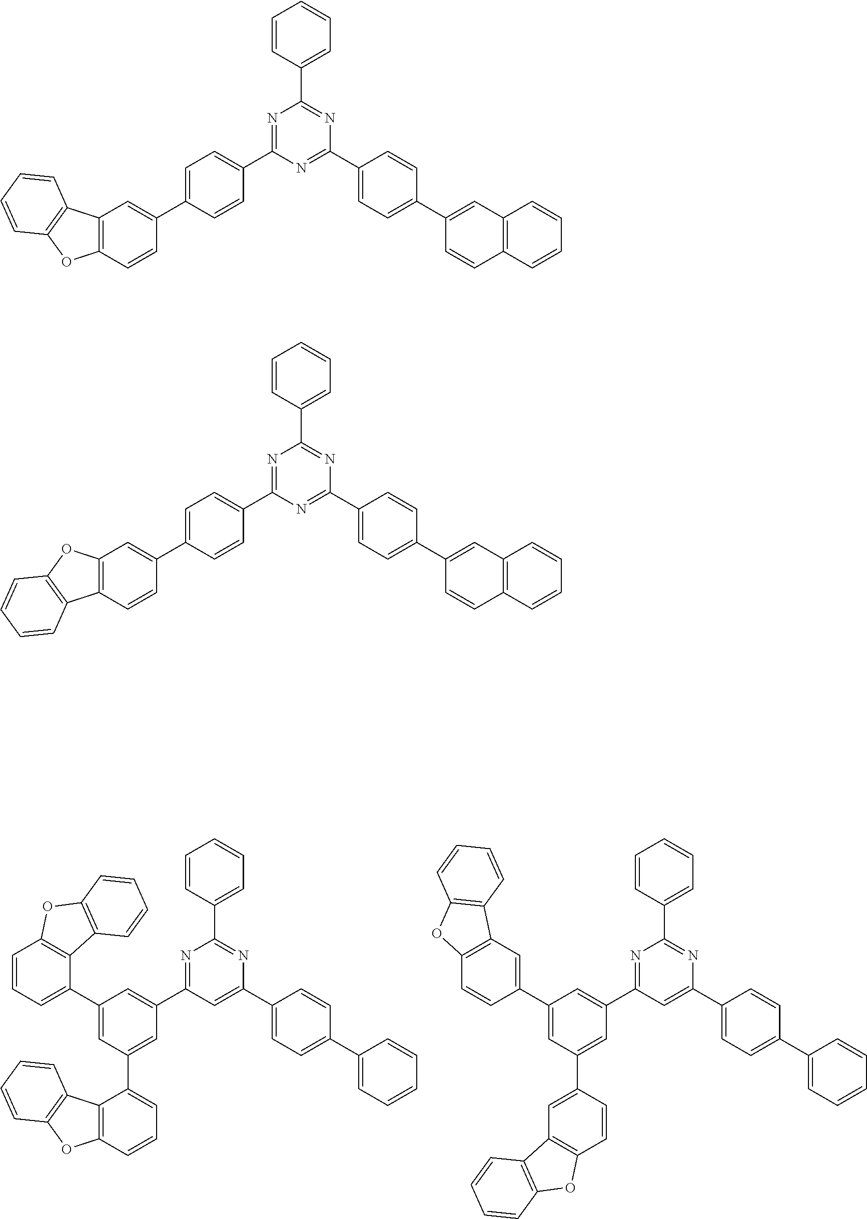 [SMILES: c1ccc(-c2nc(-c3ccc(-c4ccc5ccccc5c4)cc3)nc(-c3ccc(-c4ccc5oc6ccccc6c5c4)cc3)n2)cc1 ; c1ccc(-c2nc(-c3ccc(-c4ccc5ccccc5c4)cc3)nc(-c3ccc(-c4ccc5c(c4)oc4ccccc45)cc3)n2)cc1 ; c1ccc(-c2ccc(-c3cc(-c4cc(-c5cccc6oc7ccccc7c56)cc(-c5cccc6oc7ccccc7c56)c4)nc(-c4ccccc4)n3)cc2)cc1 ; c1ccc(-c2ccc(-c3cc(-c4cc(-c5ccc6oc7ccccc7c6c5)cc(-c5ccc6oc7ccccc7c6c5)c4)nc(-c4ccccc4)n3)cc2)cc1]

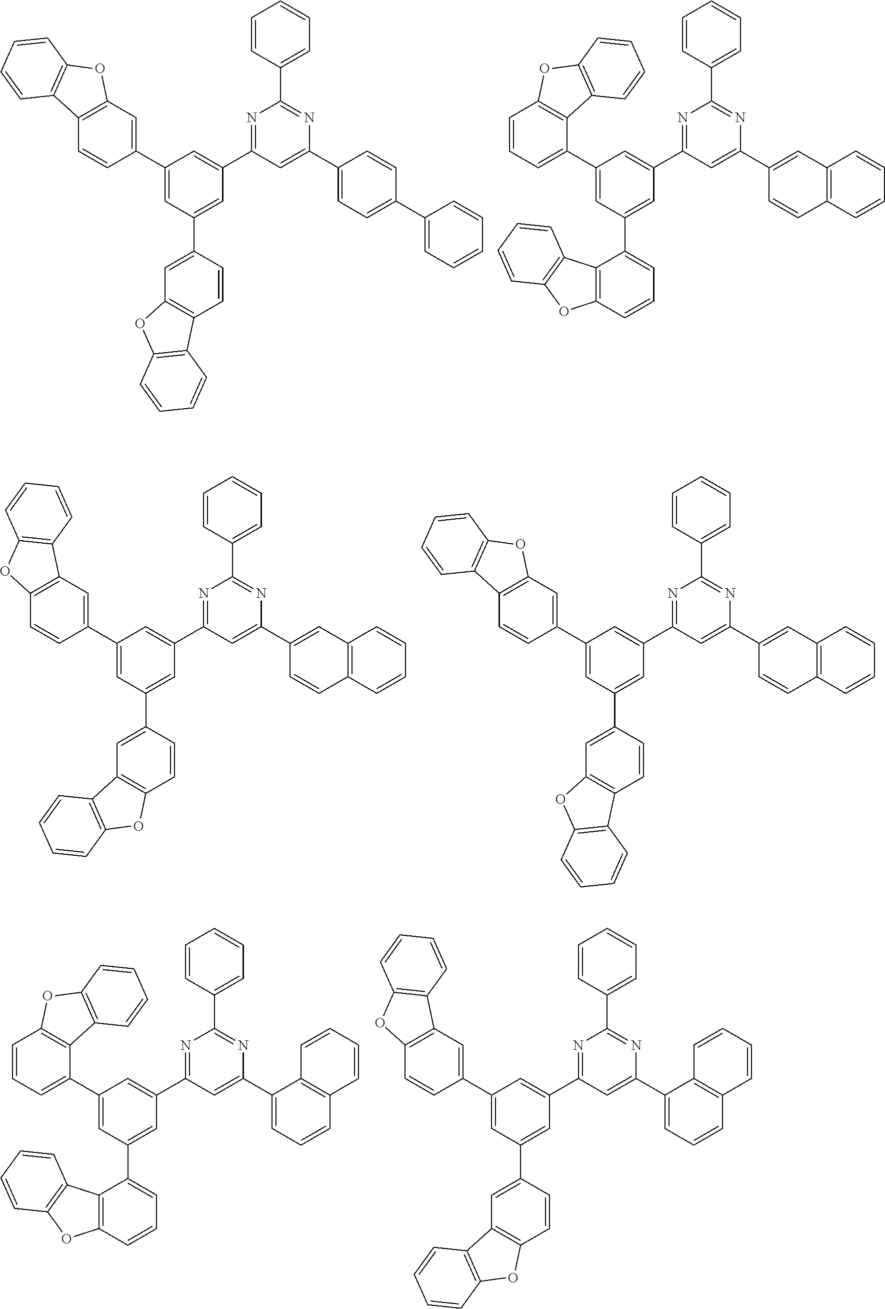

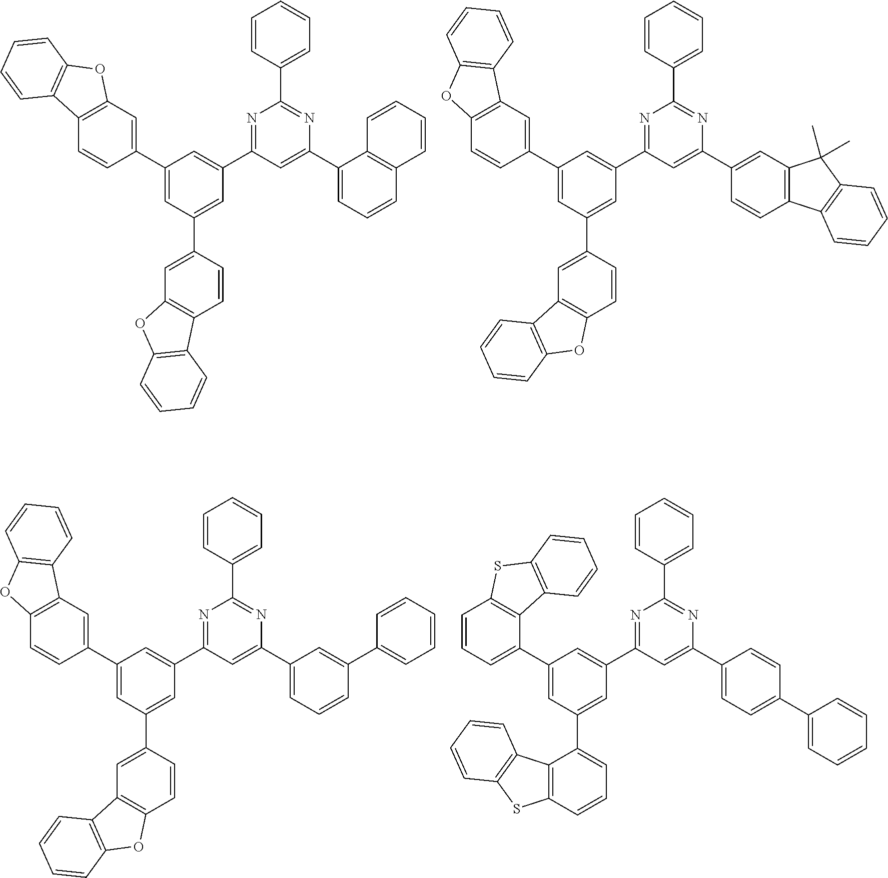

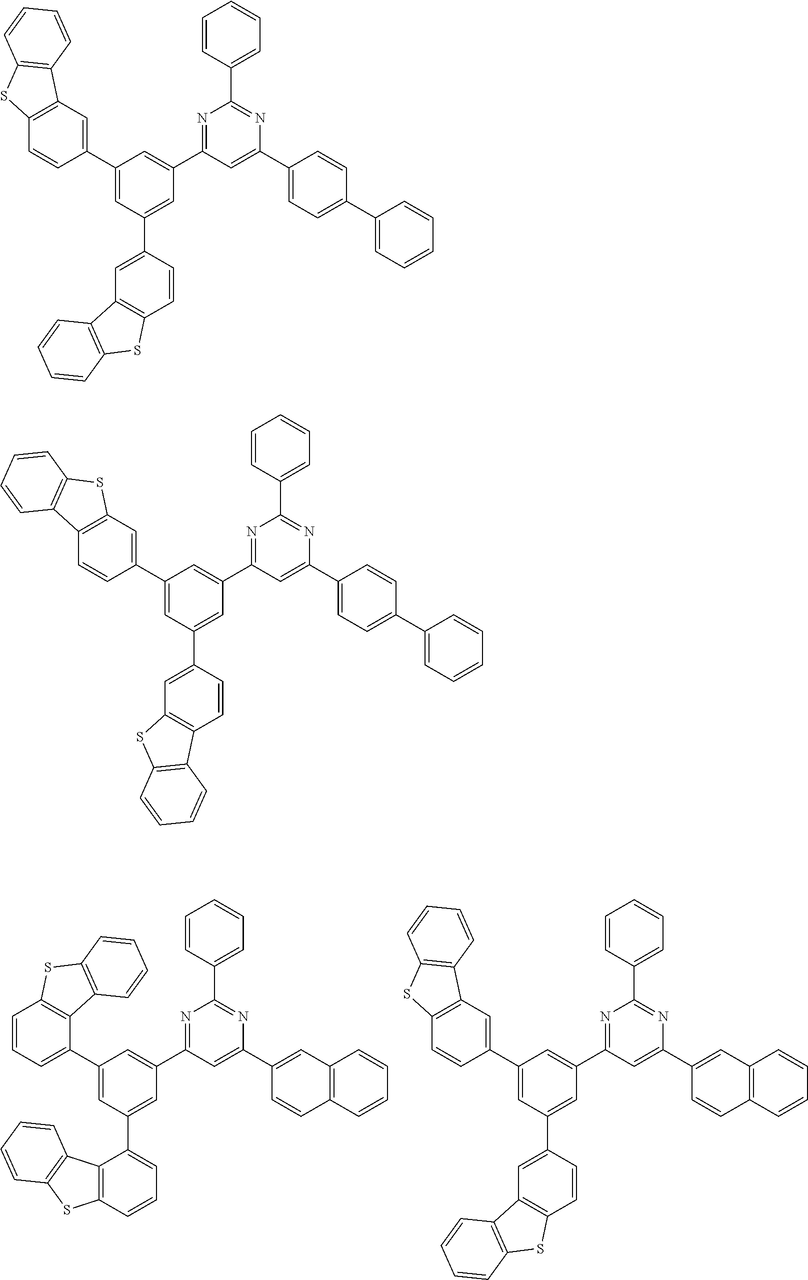 [SMILES: c1ccc(-c2ccc(-c3cc(-c4cc(-c5ccc6sc7ccccc7c6c5)cc(-c5ccc6sc7ccccc7c6c5)c4)nc(-c4ccccc4)n3)cc2)cc1 ; c1ccc(-c2ccc(-c3cc(-c4cc(-c5ccc6c(c5)sc5ccccc56)cc(-c5ccc6c(c5)sc5ccccc56)c4)nc(-c4ccccc4)n3)cc2)cc1 ; c1ccc(-c2nc(-c3cc(-c4cccc5sc6ccccc6c45)cc(-c4cccc5sc6ccccc6c45)c3)cc(-c3ccc4ccccc4c3)n2)cc1 ; c1ccc(-c2nc(-c3cc(-c4ccc5sc6ccccc6c5c4)cc(-c4ccc5sc6ccccc6c5c4)c3)cc(-c3ccc4ccccc4c3)n2)cc1]

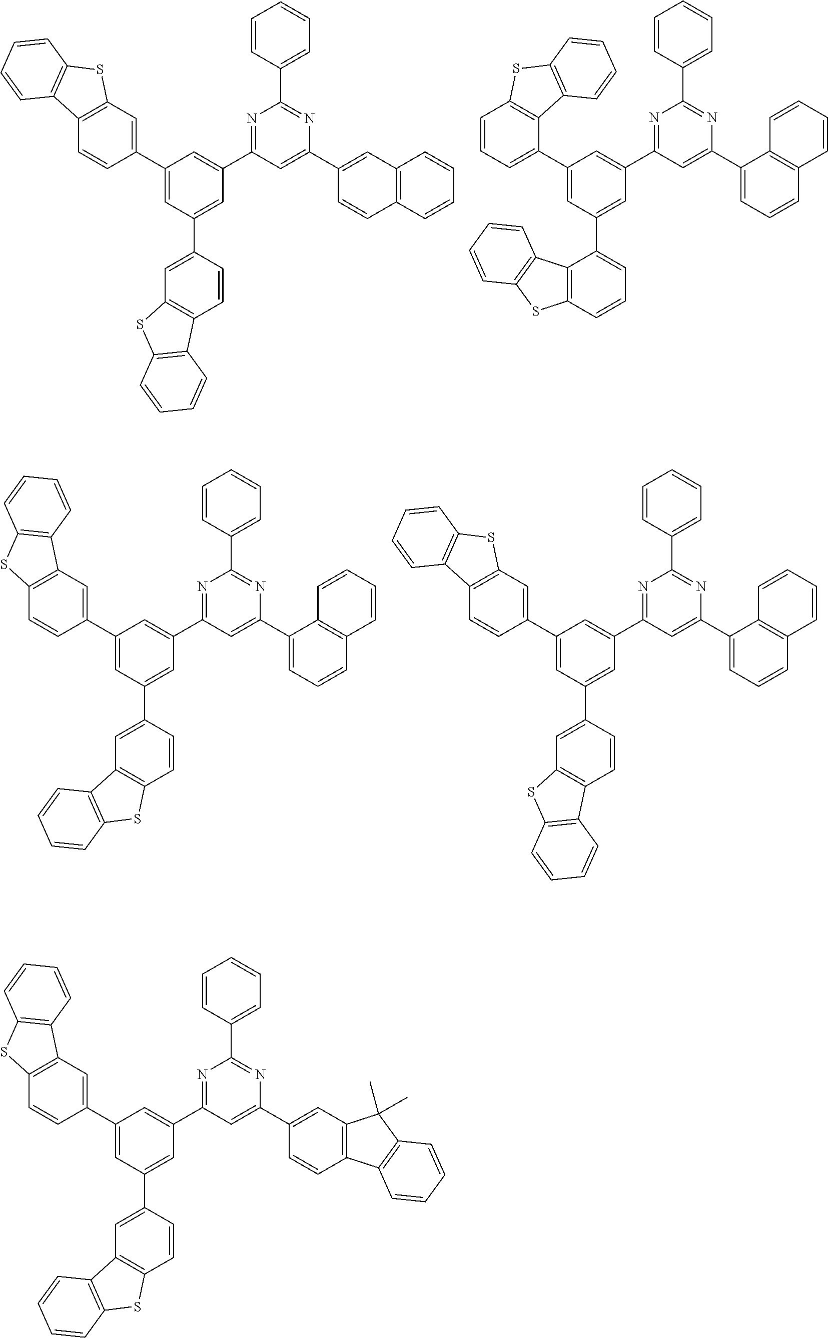

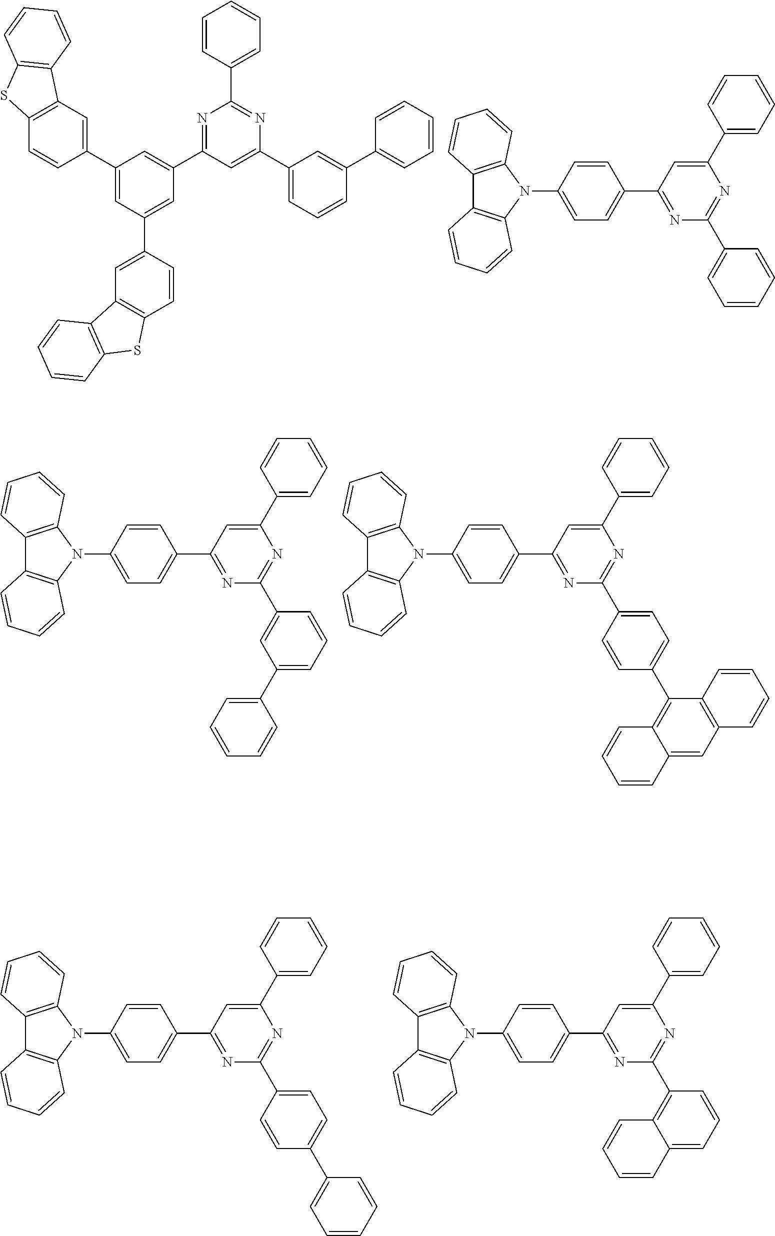

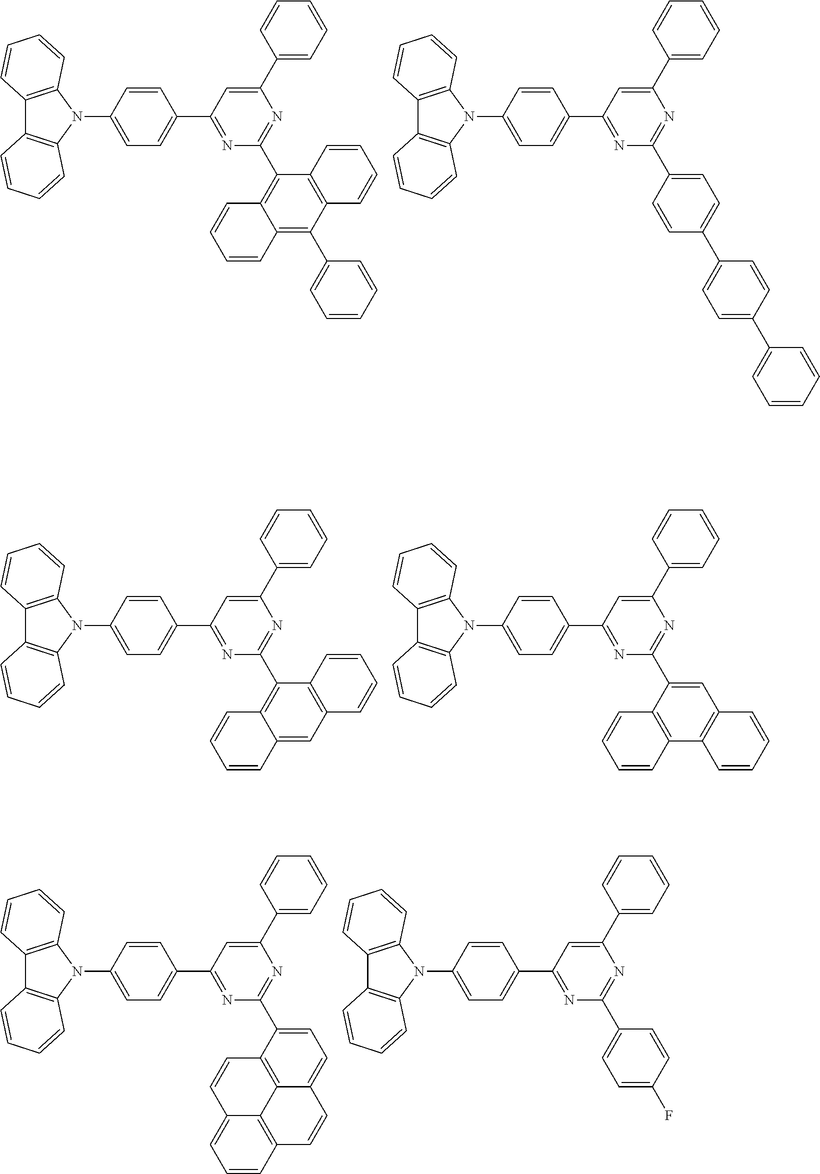

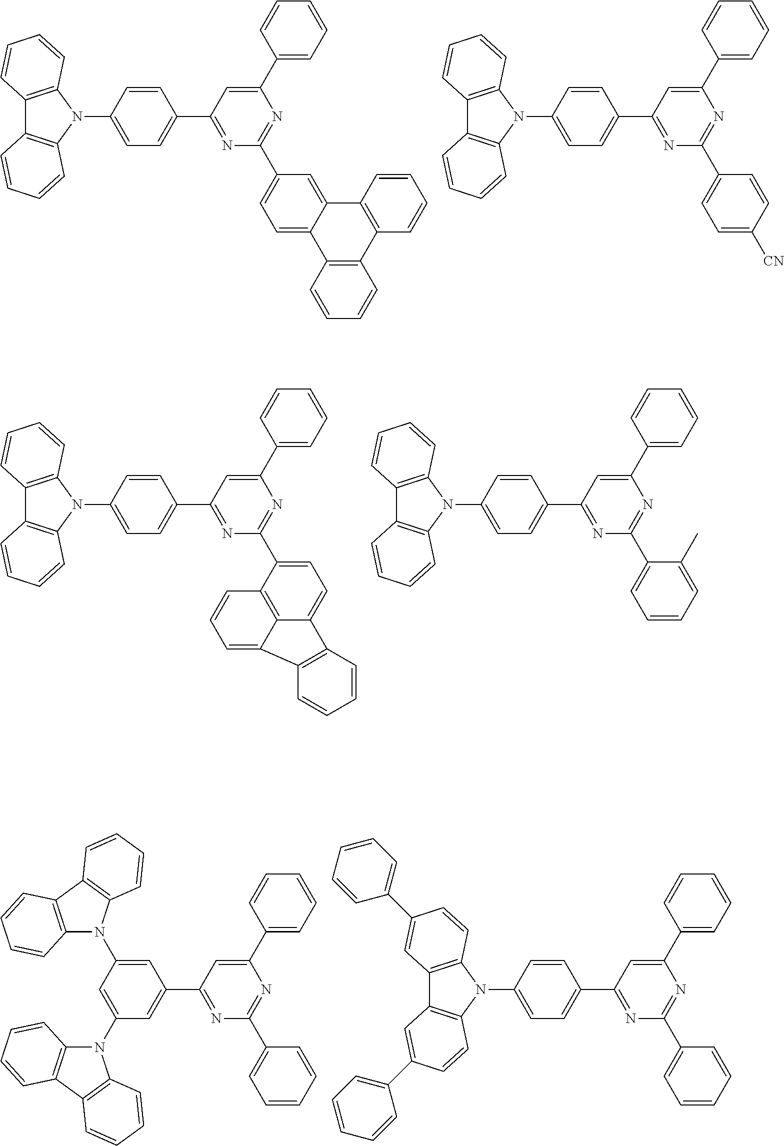

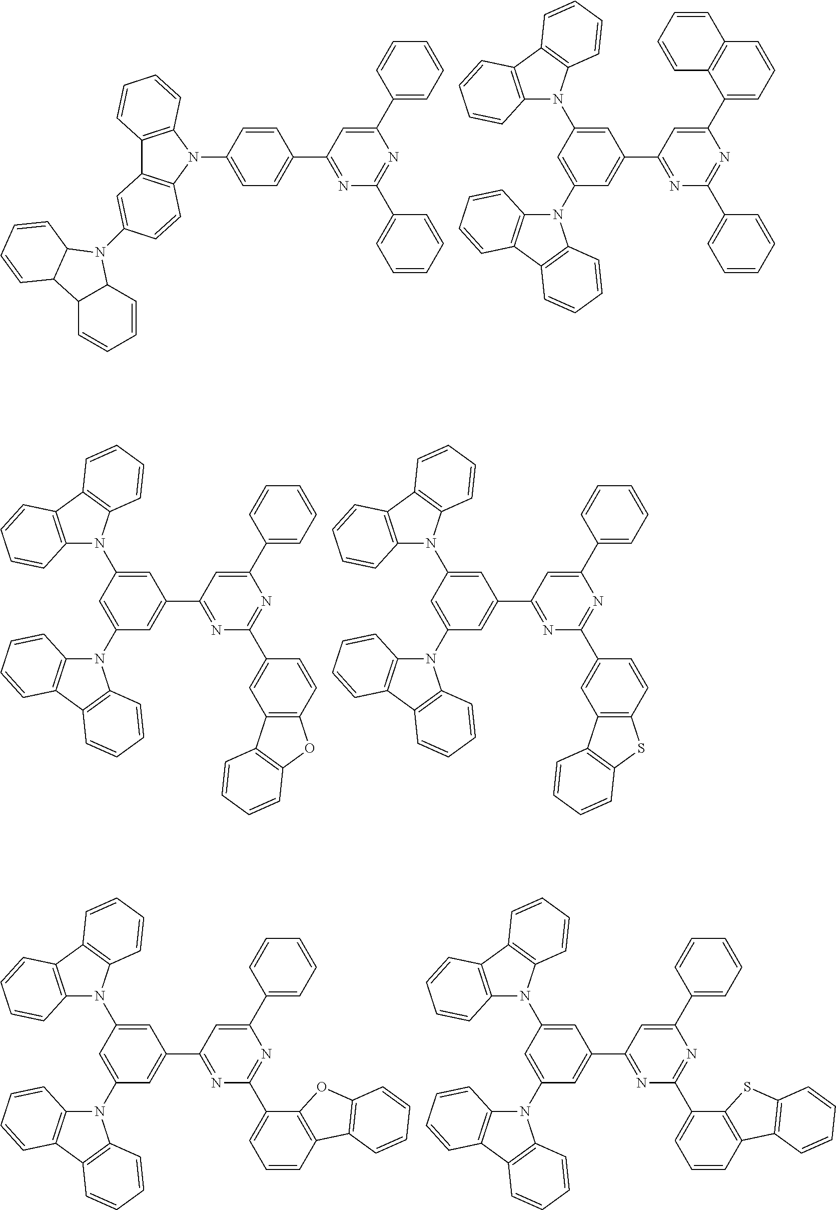

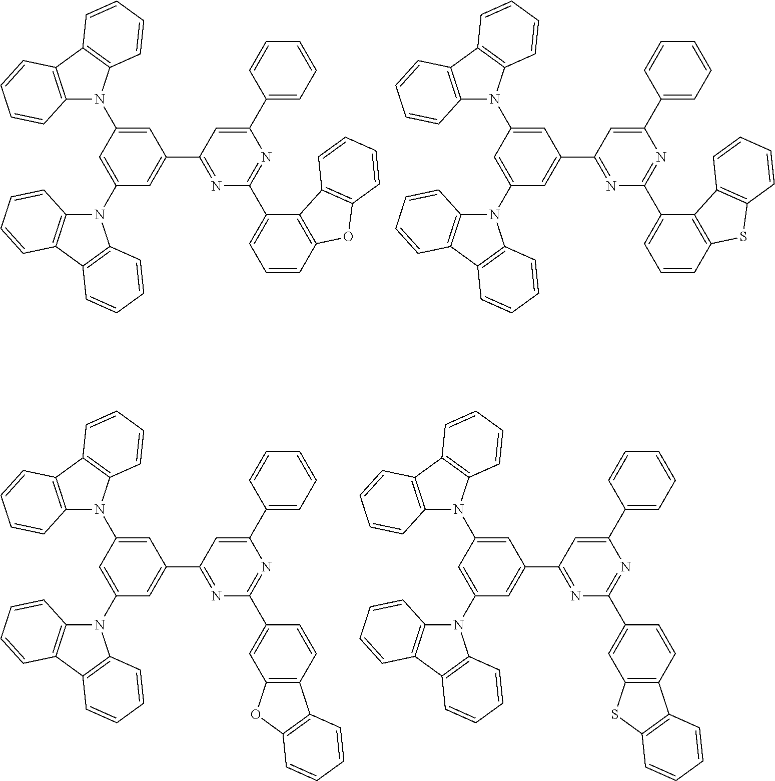

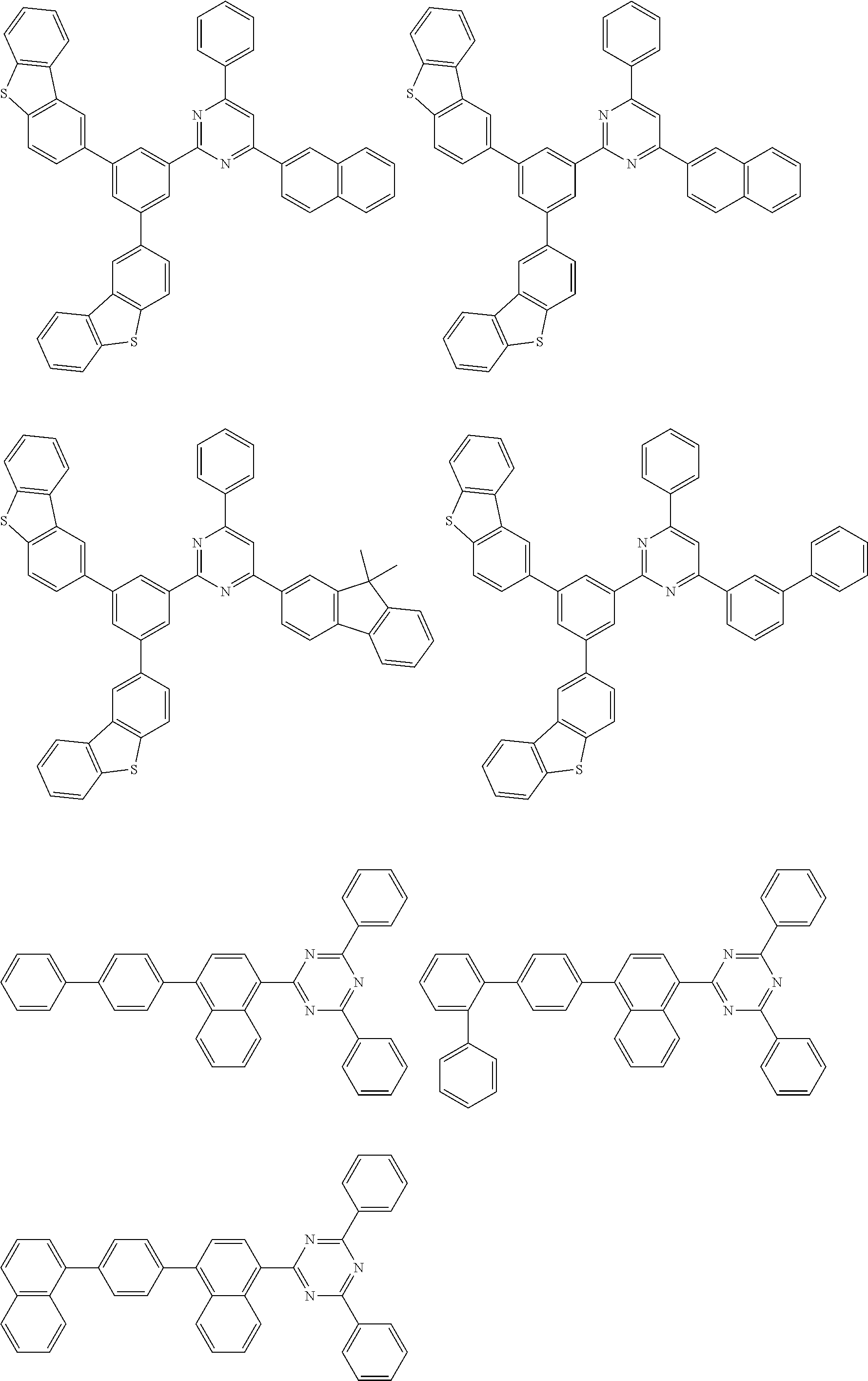

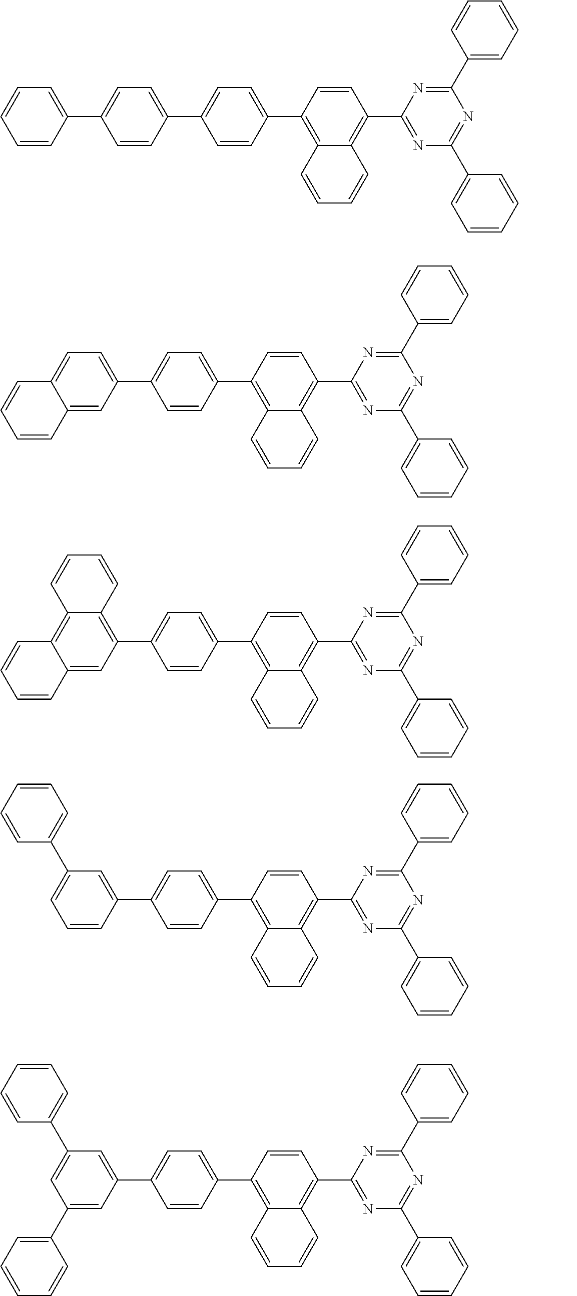

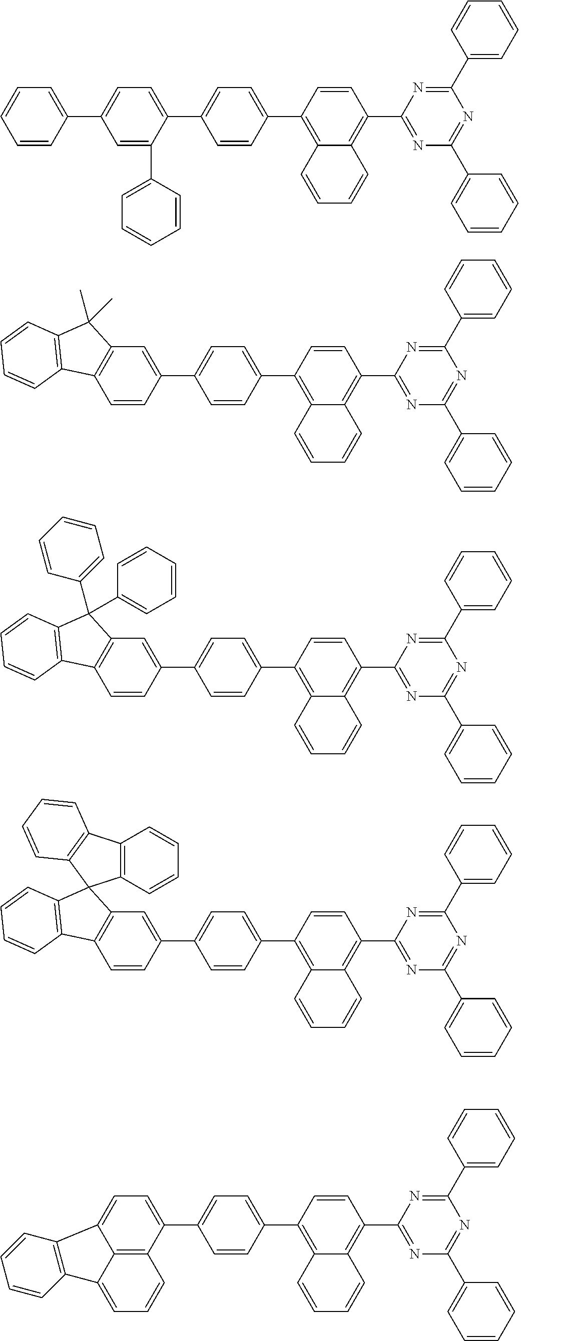

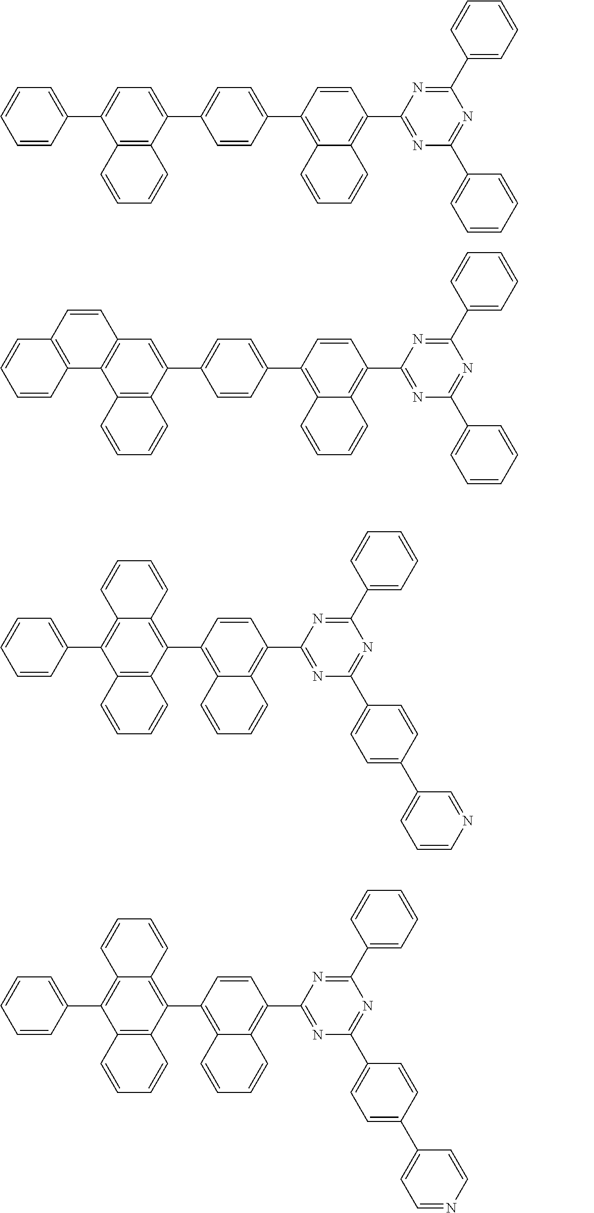

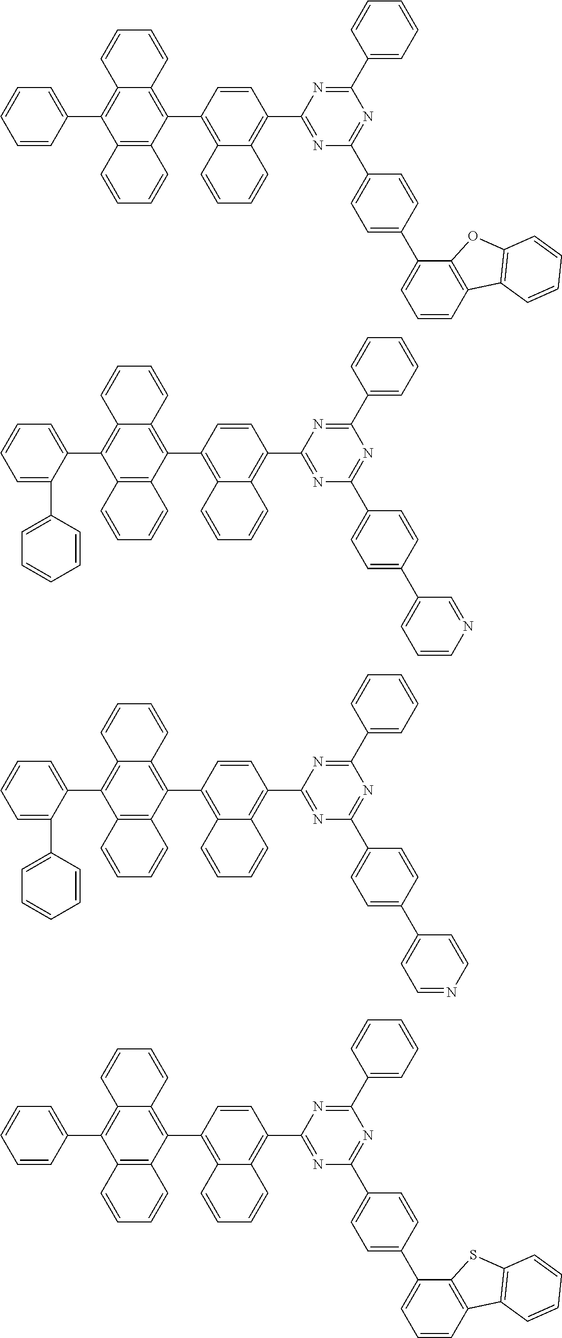

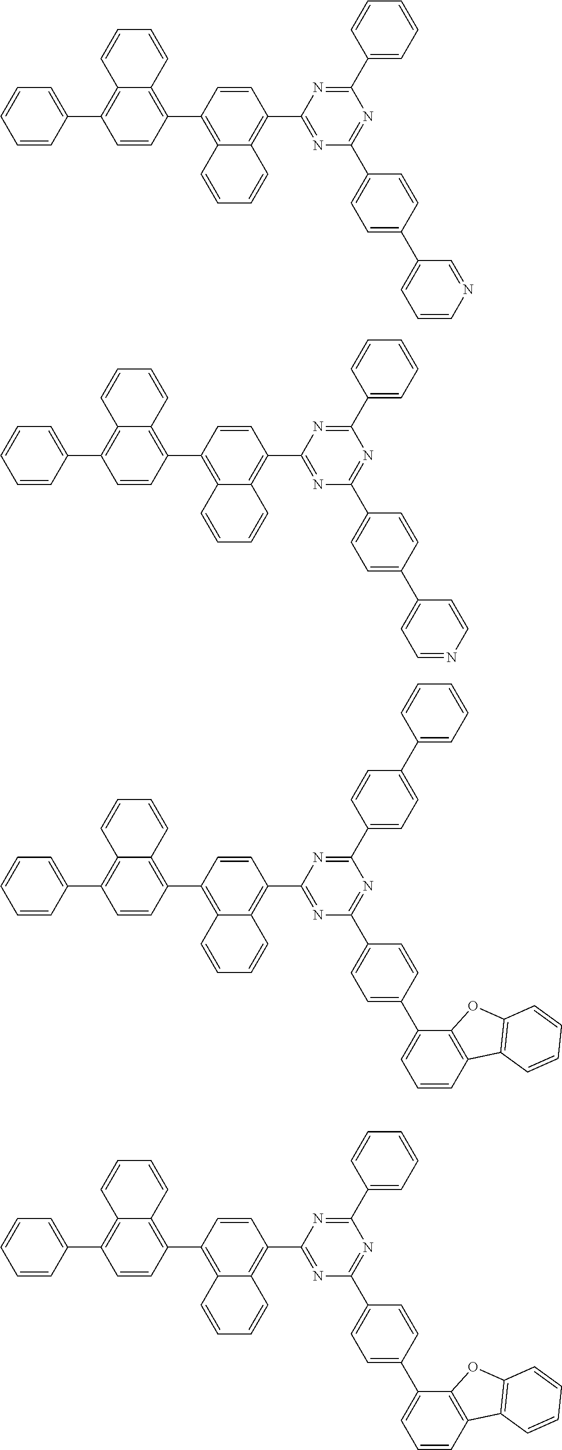

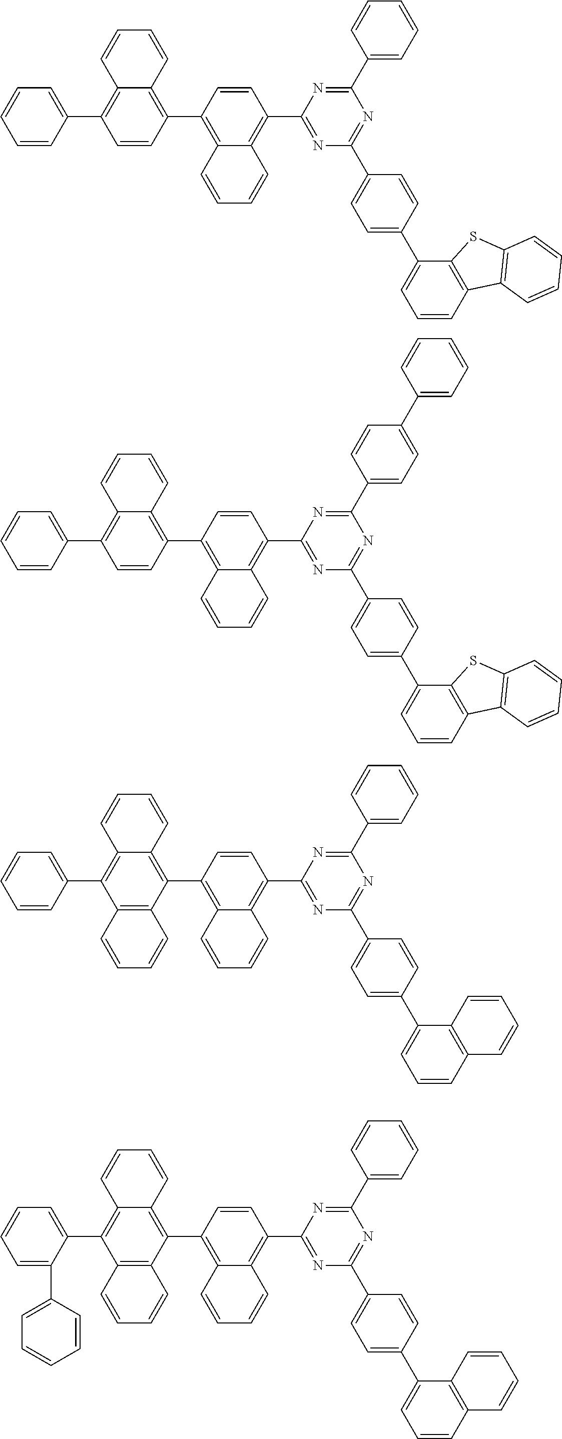

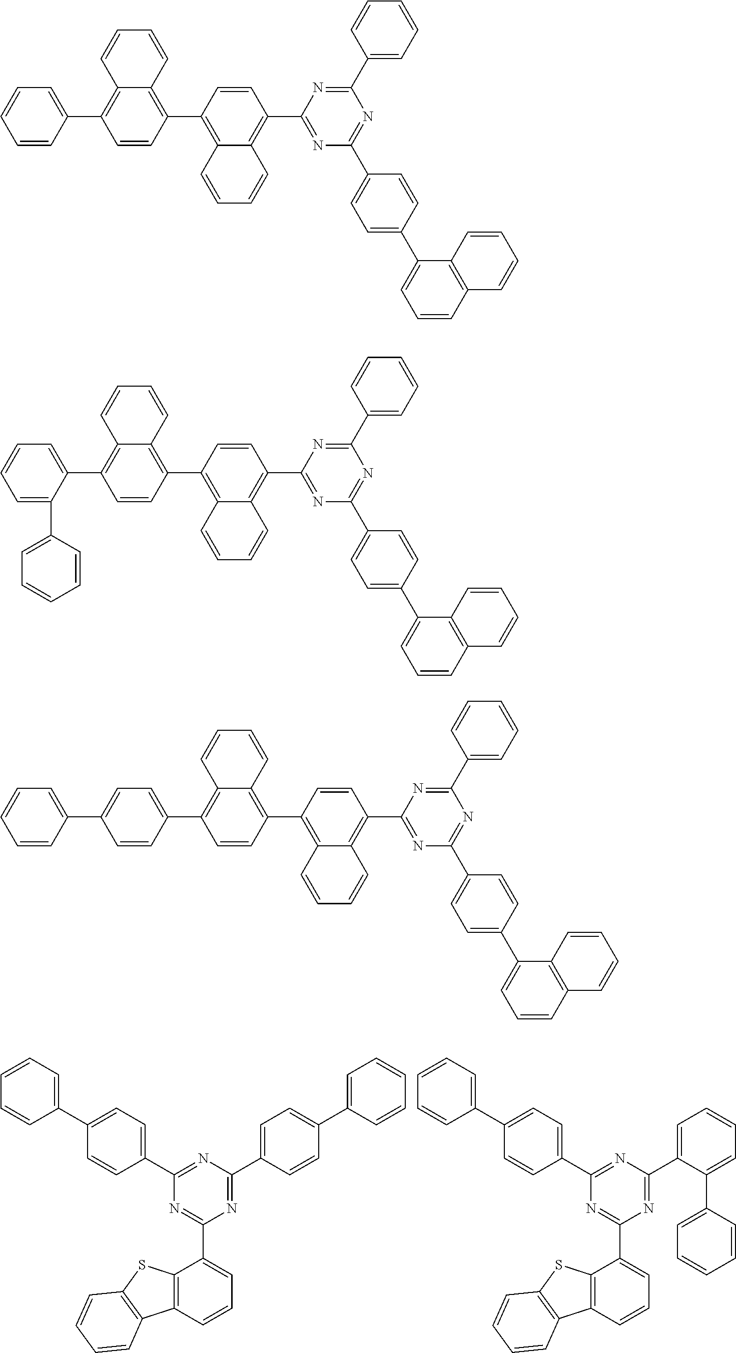

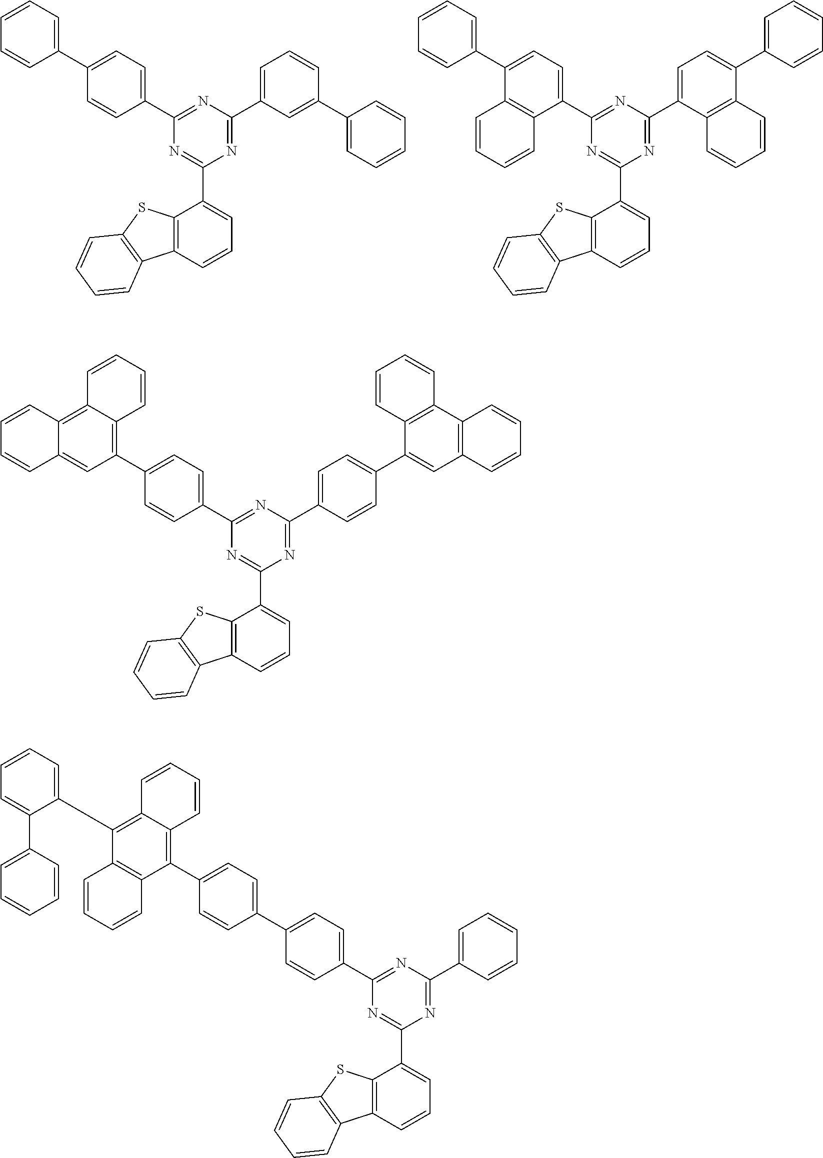 [SMILES: c1ccc(-c2ccc(-c3nc(-c4cccc(-c5ccccc5)c4)nc(-c4cccc5c4sc4ccccc45)n3)cc2)cc1 ; c1ccc(-c2ccc(-c3nc(-c4ccc(-c5ccccc5)c5ccccc45)nc(-c4cccc5c4sc4ccccc45)n3)c3ccccc23)cc1 ; c1ccc2c(c1)cc(-c1ccc(-c3nc(-c4ccc(-c5cc6ccccc6c6ccccc56)cc4)nc(-c4cccc5c4sc4ccccc45)n3)cc1)c1ccccc12 ; c1ccc(-c2nc(-c3ccc(-c4ccc(-c5c6ccccc6c(-c6ccccc6-c6ccccc6)c6ccccc56)cc4)cc3)nc(-c3cccc4c3sc3ccccc34)n2)cc1]

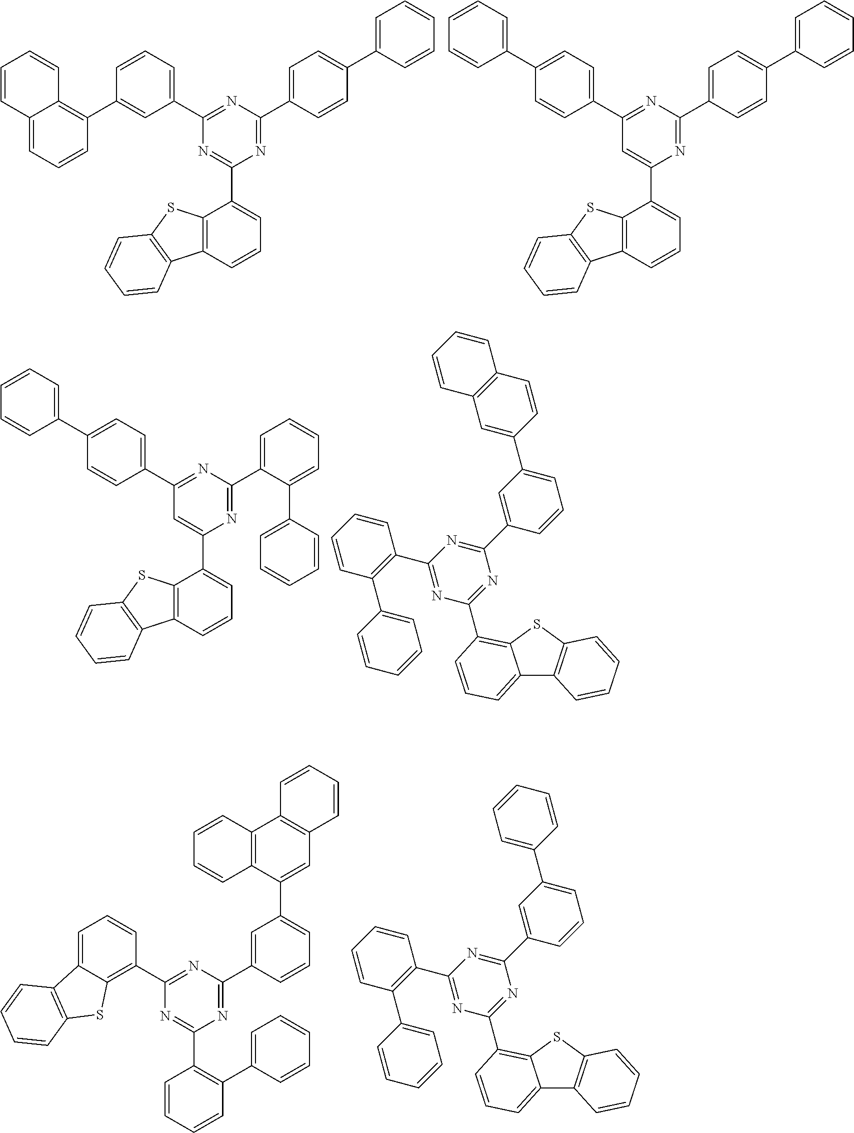

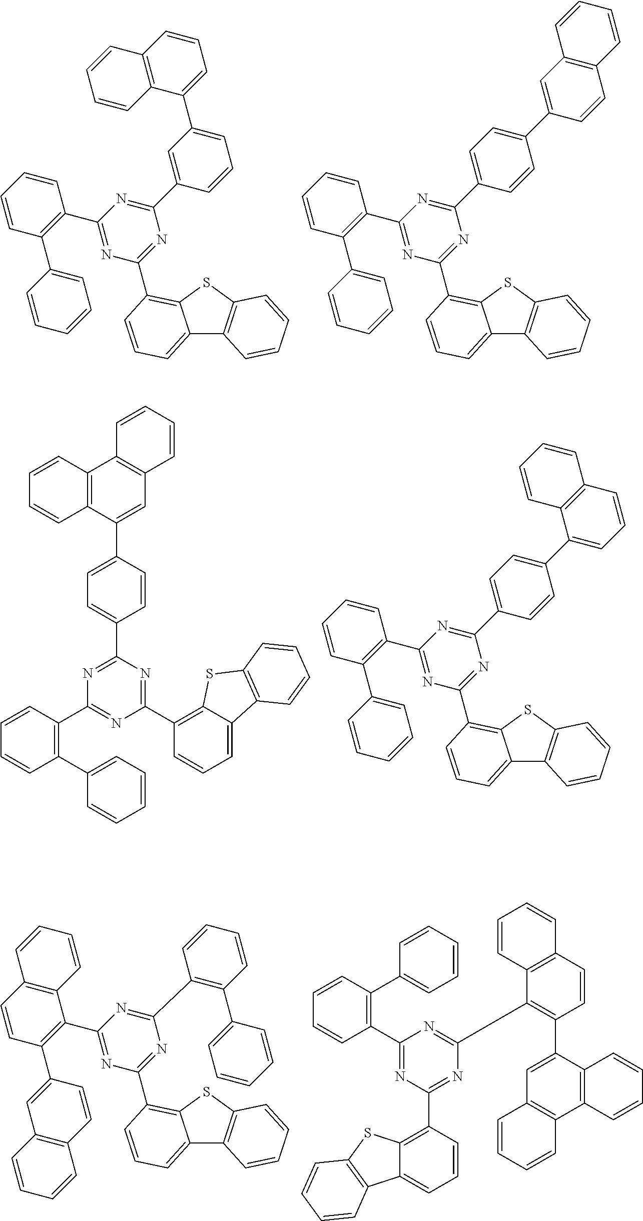

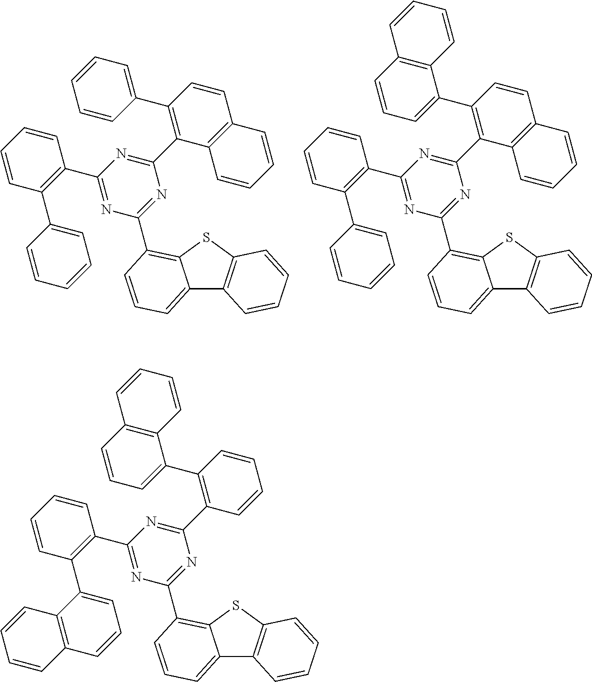

### Electron Injecting Layer

The electron injecting layer is a layer containing a highly electron-injectable substance.

For the electron injecting layer in the organic EL device according to the third exemplary embodiment, an alkali metal, an alkaline earth metal, or a compound thereof, such as lithium (Li), cesium (Cs), calcium (Ca), lithium fluoride (LiF), cesium fluoride (CsF), calcium fluoride (CaF₂), or lithium oxide (LiOx), can be used.

In addition, the alkali metal, alkaline earth metal or the compound thereof may be added to the substance exhibiting the electron transportability in use.

Specifically, for instance, magnesium (Mg) added to Alq may be used.

In this case, the electrons can be more efficiently injected from the cathode.

Alternatively, the electron injecting layer may be provided by a composite material in a form of a mixture of the organic compound and the electron donor.

Such a composite material exhibits excellent electron injectability and electron transportability since electrons are generated in the organic compound by the electron donor.

In this case, the organic compound is preferably a material excellent in transporting the generated electrons. Specifically, the above examples (e.g., the metal complex and the hetero aromatic compound) of the substance forming the electron transporting layer are usable.

As the electron donor, any substance exhibiting electron donating property to the organic compound is usable.

Specifically, the electron donor is preferably alkali metal, alkaline earth metal and rare earth metal such as lithium, cesium, magnesium, calcium, erbium and ytterbium.

The electron donor is also preferably alkali metal oxide and alkaline earth metal oxide such as lithium oxide, calcium oxide, and barium oxide.

Moreover, a Lewis base such as magnesium oxide is usable.

Further, the organic compound such as tetrathiafulvalene (abbreviation: TTF) is usable.

### Layer Formation Method

A method for forming each layer of the organic EL device in the exemplary embodiment is subject to no limitation except for the above particular description. However, known methods of dry film-forming such as vacuum deposition, sputtering, plasma or ion plating and wet film-forming such as spin coating, dipping, flow coating or ink-jet are applicable.

### Film Thickness

The thickness of each organic layer of the organic EL device according to the third exemplary embodiment is not limited unless otherwise specified above.

In general, the thickness preferably ranges from several nanometers to 1 μm because an excessively small film thickness is likely to cause defects (e.g. pin holes) and an excessively large thickness leads to the necessity of applying high voltage and consequent reduction in efficiency.

### Emission Wavelength of Organic EL Device

The organic electroluminescence device according to the third exemplary embodiment preferably emits light with a maximum peak wavelength of 500 nm or less when the device is driven.

The organic electroluminescence device according to the third exemplary embodiment more preferably emits light with a maximum peak wavelength in a range from 430 nm to 480 nm when the device is driven.

The organic electroluminescence device according to the third exemplary embodiment still more preferably emits light with a maximum peak wavelength of 470 nm or less, still further more preferably emits light with a maximum peak wavelength of 465 nm or less when the device is driven.

The organic electroluminescence device according to the third exemplary embodiment still further more preferably emits light with a maximum peak wavelength of 440 nm or more, still further more preferably emits light with a maximum peak wavelength of 445 nm or more when the device is driven.

The maximum peak wavelength of light emitted by the organic EL device when the device is driven is measured in the following manner.

Voltage is applied to the organic EL device such that a current density becomes 10 mA/cm², where spectral radiance spectrum is measured by a spectroradiometer CS-2000 (produced by Konica Minolta, Inc.).

A peak wavelength of an emission spectrum, a luminous intensity of which is the maximum in the obtained spectral radiance spectrum, is measured and defined as the maximum peak wavelength (unit: nm).

### Fourth Exemplary Embodiment

### Electronic Device

An electronic device according to a fourth exemplary embodiment is installed with any one of the organic EL devices according to the above exemplary embodiments.

Examples of the electronic device include a display device and a light-emitting unit.

Examples of the display device include a display component (e.g., an organic EL panel module), TV, mobile μhone, tablet and personal computer.

Examples of the light-emitting unit include an illuminator and a vehicle light.

The light-emitting unit can be used in the display device, and can be used as, for instance, a backlight of the display device.

### Modification of Embodiment(s)

The scope of the invention is not limited by the above-described exemplary embodiments but includes any modification and improvement as long as such modification and improvement are compatible with the invention.

For instance, the number of emitting layers included in the organic EL device need not necessarily be one or two, and three or more emitting layers may be layered.

When the organic EL device includes two or more emitting layers, at least one emitting layer (the first emitting layer) needs to satisfy the conditions described in the above exemplary embodiments.

For instance, the rest of the emitting layers may be a fluorescent emitting layer or a phosphorescent emitting layer with use of emission caused by electron transfer from the triplet excited state directly to the ground state.

When the organic EL device includes a plurality of emitting layers, these emitting layers may be mutually adjacently provided, or may form a so-called tandem organic EL device, in which a plurality of emitting units are layered via an intermediate layer.

Specific structure, shape and the like of the components in the invention may be designed in any manner as long as an object of the invention can be achieved.

### EXAMPLES

The invention will now be described in more detail with reference to Examples.

The invention is not limited to these Examples.

### Compounds

Structures of compounds represented by the formula (1) used to produce organic EL devices of Examples 1 to 4 are shown below.

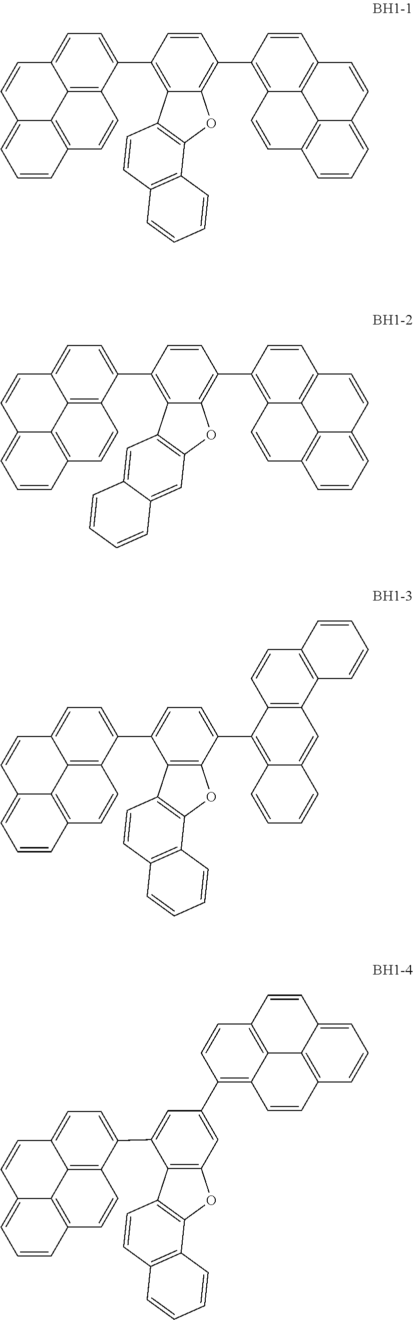 [SMILES: c1ccc2c(c1)ccc1c2oc2c(-c3ccc4ccc5cccc6ccc3c4c56)ccc(-c3ccc4ccc5cccc6ccc3c4c56)c21 ; c1ccc2cc3c(cc2c1)oc1c(-c2ccc4ccc5cccc6ccc2c4c56)ccc(-c2ccc4ccc5cccc6ccc2c4c56)c13 ; c1ccc2c(-c3ccc(-c4ccc5ccc6cccc7ccc4c5c67)c4c3oc3c5ccccc5ccc34)c3ccc4ccccc4c3cc2c1 ; c1ccc2c(c1)ccc1c2oc2cc(-c3ccc4ccc5cccc6ccc3c4c56)cc(-c3ccc4ccc5cccc6ccc3c4c56)c21]

Structures of comparative compounds used to produce organic EL devices of Comparative Examples 1 to 3 are shown below.

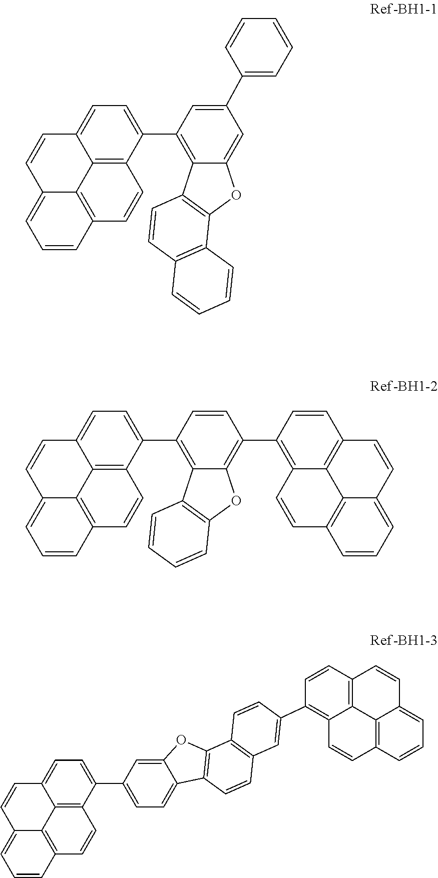 [SMILES: c1ccc(-c2cc(-c3ccc4ccc5cccc6ccc3c4c56)c3c(c2)oc2c4ccccc4ccc23)cc1 ; c1cc2ccc3ccc(-c4ccc(-c5ccc6ccc7cccc8ccc5c6c78)c5c4oc4ccccc45)c4ccc(c1)c2c34 ; c1cc2ccc3ccc(-c4ccc5c(ccc6c7ccc(-c8ccc9ccc%10cccc%11ccc8c9c%10%11)cc7oc56)c4)c4ccc(c1)c2c34]

Structures of other compounds used to produce organic EL devices in Examples 1 to 4 and Comparatives 1 to 3 are shown below.

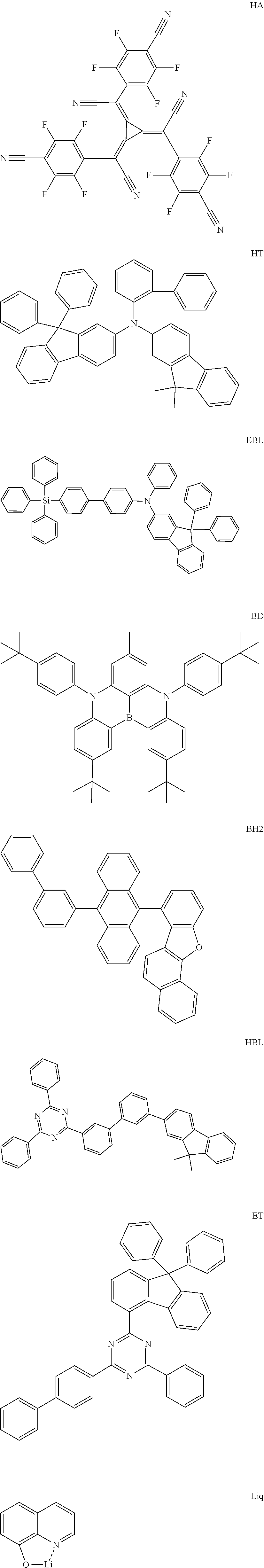

### Production of Organic EL Device

The organic EL devices were produced and evaluated as follows.

### Example 1

A glass substrate (size: 25 mm×75 mm×1.1 mm thick, produced by Geomatec Co., Ltd.) having an ITO transparent electrode (anode) was ultrasonic-cleaned in isopropyl alcohol for five minutes, and then UV-ozone-cleaned for 30 minutes.

The film thickness of the ITO transparent electrode was 130 nm.

After the glass substrate was cleaned, the glass substrate was mounted on a substrate holder of a vacuum evaporation apparatus. First, a compound HT and a compound HA were co-deposited on a surface of the glass substrate, where the transparent electrode line was provided, to cover the transparent electrode, thereby forming a 10-nm-thick hole injecting layer.

In the hole injecting layer, the ratio of the compound HT was 90 mass %, and the ratio of the compound HA was 10 mass %.

Next, the compound HT was vapor-deposited on the hole injecting layer to form an 85-nm-thick first hole transporting layer.

Next, a compound EBL was vapor-deposited on the first hole transporting layer to form a 5-nm-thick second hole transporting layer (sometimes referred to as an electron blocking layer).

Next, a compound BH1-1 (first host material) and a compound BD (first emitting compound) were co-deposited on the second hole transporting layer to form a 5-nm-thick first emitting layer.

In the first emitting layer, the ratio of the compound BH1-1 was 98 mass %, and the ratio of the compound BD was 2 mass %.

Next, a compound BH2 (second host material) and the compound BD (second emitting compound) were co-deposited on the first emitting layer to form a 15-nm-thick second emitting layer.

In the second emitting layer, the ratio of the compound BH2 was 98 mass %, and the ratio of the compound BD was 2 mass %.

Next, a compound HBL was vapor-deposited on the second emitting layer to form a 5-nm-thick first electron transporting layer (sometimes referred to as a hole blocking layer).

Next, the compound ET and a compound Liq were co-deposited on the first electron transporting layer to form a 25-nm-thick second electron transporting layer.

In the second electron transporting layer, the ratio of the compound ET was 50 mass %, and the ratio of the compound Liq was 50 mass %.

Liq is an abbreviation of (8-quinolinolato)lithium ((8-Quinolinolato)lithium).

Next, the compound Liq was vapor-deposited on the second electron transporting layer to form a 1-nm-thick electron injecting layer.

Next, metal Al was vapor-deposited on the electron injecting layer to form an 80-nm-thick cathode.

An arrangement of the device of Example 1 is roughly shown as follows.

ITO(130)/HT:HA(10,90:10%)/HT(85)/EBL(5)/BH1-1:BD(5,98%:2%)/BH2:BD(15,98%:2%)/HBL(5)/ET:Liq(25,50%:50%)/Liq(1)/Al(80)

In the roughly shown arrangement of the device, numerals in parentheses each represent a film thickness (unit: nm).

The numerals (90%:10%) represented by percentage in the same parentheses indicate a ratio (mass %) between the compound HT and the compound HA in the hole injecting layer. The numerals (98%:2%) represented by percentage in the same parentheses indicate a ratio (mass %) between the host material (compound BH1-1 or BH2) and the emitting compound (compound BD) in the first emitting layer or the second emitting layer. The numerals (50%:50%) represented by percentage in the same parentheses indicate a ratio (mass %) between the compound ET and the compound Liq in the second electron transporting layer.

Similar notations apply to the description below.

### Examples 2 to 4

Organic EL devices of Examples 2 to 4 were produced in the same manner as the organic EL device of Example 1 except that the compound BH1-1 as the first host material used to form the first emitting layer was changed to the compounds shown in Table 1.

### Comparatives 1 to 3

Organic EL devices of Comparative Examples 1 to 3 were produced in the same manner as the organic EL device of Example 1 except that the compound BH1-1 as the first host material used to form the first emitting layer was changed to the compounds shown in Table 1.

### Evaluation of Organic EL Devices

The organic EL devices produced were evaluated as follows.

Table 1 shows the evaluation results.

Table 1 also shows the singlet energy S₁ and the triplet energy T₁ of the compounds used for the emitting layers in Examples and Comparative Examples.

### CIE1931 Chromaticity

Voltage was applied to the organic EL device such that a current density was 10 mA/cm², where CIE1931 chromaticity coordinates (x, y) were measured with a spectroradiometer CS-2000 (produced by Konica Minolta Holding, Inc.).

Table 1 shows the values of CIEy.

### Drive Voltage

Current was applied between the anode and the cathode of the organic EL device such that a current density was 10 mA/cm², where a voltage (unit: V) was measured.

### External Quantum Efficiency EQE

Voltage was applied to the organic EL device such that a current density was 10 mA/cm², where a spectral radiance spectrum was measured with a spectroradiometer CS-2000 (produced by Konica Minolta Holding, Inc.).

The external quantum efficiency EQE (unit: %) was calculated based on the obtained spectral radiance spectra, assuming that the spectra was provided under a Lambertian radiation.

### Lifetime LT95

Voltage was applied to the organic EL device produced such that a current density was 50 mA/cm², and the time (LT95 (unit: h)) until the luminance became 95% of the initial luminance was measured as a lifetime.

The luminance was measured using a spectroradiometer CS-2000 (produced by Konica Minolta Holding, Inc.).

TABLE 1

| | First emitting layer | | | | | | Second emitting layer | | | | | | | |
|---|---|---|---|---|---|---|---|---|---|---|---|---|---|---|
| | First | | | First emitting | | | Second | | | Second | Device evaluation | | | |
| | host material | | | compound | | | host material | | | emitting | | | Drive | |
| | | S₁ | T₁ | | S₁ | T₁ | | S₁ | T₁ | compound | | EQE | voltage | LT95 |
| | Name | [eV] | [eV] | Name | [eV] | [eV] | Name | [eV] | [eV] | Name | CIEy | [%] | [V] | [h] |
| Example 1 | BH1-1 | 3.14 | 2.09 | BD | 2.71 | 2.64 | BH2 | 3.12 | 1.85 | BD | 0.087 | 10.7 | 3.25 | 96 |
| Example 2 | BH1-2 | 3.10 | 2.09 | BD | 2.71 | 2.64 | BH2 | 3.12 | 1.85 | BD | 0.088 | 10.5 | 3.21 | 91 |
| Example 3 | BH1-3 | 3.22 | 2.07 | BD | 2.71 | 2.64 | BH2 | 3.12 | 1.85 | BD | 0.085 | 10.7 | 3.28 | 80 |
| Example 4 | BH1-4 | 3.12 | 2.09 | BD | 2.71 | 2.64 | BH2 | 3.12 | 1.85 | BD | 0.087 | 10.2 | 3.21 | 102 |
| Comparative | Ref-BH1-1 | 3.26 | 2.10 | BD | 2.71 | 2.64 | BH2 | 3.12 | 1.85 | BD | 0.080 | 10.1 | 3.22 | 63 |
| Example 1 | | | | | | | | | | | | | | |
| Comparative | Ref-BH1-2 | 3.16 | 2.09 | BD | 2.71 | 2.64 | BH2 | 3.12 | 1.85 | BD | 0.088 | 10.8 | 3.22 | 71 |
| Example 2 | | | | | | | | | | | | | | |
| Comparative | Ref-BH1-3 | 3.17 | 2.09 | BD | 2.71 | 2.64 | BH2 | 3.12 | 1.85 | BD | 0.091 | 10.1 | 3.22 | 85 |
| Example 3 | | | | | | | | | | | | | | |

In the organic EL devices of Examples 1 to 4, the compound represented by the formula (1) was used as a host material.

Consequently, the organic EL devices of Examples 1 to 4 had a longer lifetime LT95 than the organic EL devices of Comparative Examples 1 and 2.

The organic EL devices of Examples 1, 2, and 4 had a longer lifetime LT95 than the organic EL device of Comparative Example 3.

The organic EL device of Example 3 had a prolonged lifetime while maintaining a chromaticity comparable to those in Comparative Example 1 and Comparative Example 2.

In each of Examples 1 to 4, the CIEy was less than 0.091, meaning that deterioration in chromaticity was inhibited compared with Comparative Example 3 using a compound in which aryls were bonded to different rings of naphthobenzofuran.

### Evaluation of Compounds

### Triplet Energy T₁

A measurement target compound was dissolved in EPA (diethyl ether:isopentane:ethanol=5:5:2 (volume ratio)) at a concentration of 10 μmol/L to obtain a solution, and the solution was put in a quartz cell to provide a measurement sample.

A phosphorescent spectrum (ordinate axis: phosphorescent luminous intensity, abscissa axis: wavelength) of the measurement sample was measured at a low temperature (77K).

A tangent was drawn to the rise of the phosphorescent spectrum close to the short-wavelength region. An energy amount was calculated by a conversion equation (F1) below based on a wavelength value λ_{edge} [nm] at an intersection of the tangent and the abscissa axis and was defined as triplet energy T₁.

It should be noted that the triplet energy T₁ may have an error of about plus or minus 0.02 eV depending on measurement conditions.

T₁ [eV]=1239.85/λ_{edge} Conversion Equation (F1):

The tangent to the rise of the phosphorescence spectrum close to the short-wavelength region is drawn as follows.

While moving on a curve of the phosphorescence spectrum from the short-wavelength region to the local maximum value closest to the short-wavelength region among the local maximum values of the phosphorescence spectrum, a tangent is checked at each point on the curve toward the long-wavelength of the phosphorescence spectrum.

An inclination of the tangent is increased along the rise of the curve (i.e., a value of the ordinate axis is increased).

A tangent drawn at a point of the local maximum inclination (i.e., a tangent at an inflection point) is defined as the tangent to the rise of the phosphorescence spectrum close to the short-wavelength region.

A local maximum point where a peak intensity is 15% or less of the maximum peak intensity of the spectrum is not counted as the above-mentioned local maximum peak intensity closest to the short-wavelength region. The tangent drawn at a point that is closest to the local maximum peak intensity closest to the short-wavelength region and where the inclination of the curve is the local maximum is defined as a tangent to the rise of the phosphorescence spectrum close to the short-wavelength region.

For phosphorescence measurement, a spectrophotofluorometer body F-4500 produced by Hitachi High-Technologies Corporation was used.

### Singlet Energy S₁

A toluene solution of a measurement target compound at a concentration of 10 μmol/L is prepared and put in a quartz cell. An absorption spectrum (ordinate axis: absorption intensity, abscissa axis: wavelength) of the thus-obtained sample is measured at a normal temperature (300K).

A tangent was drawn to the fall of the absorption spectrum close to the long-wavelength region, and a wavelength value λ_{edge} (nm) at an intersection of the tangent and the abscissa axis was assigned to a conversion equation (F2) below to calculate singlet energy.

S₁ [eV]=1239.85/λ_{edge} Conversion Equation (F2):

A spectrophotometer (U3310 produced by Hitachi, Ltd.) was used for measuring absorption spectrum.

The tangent to the fall of the absorption spectrum close to the long-wavelength region is drawn as follows.

While moving on a curve of the absorption spectrum from the local maximum value closest to the long-wavelength region, among the local maximum values of the absorption spectrum, in a long-wavelength direction, a tangent at each point on the curve is checked.

An inclination of the tangent is decreased and increased in a repeated manner as the curve falls (i.e., a value of the ordinate axis is decreased).

A tangent drawn at a point where the inclination of the curve is the local minimum closest to the long-wavelength region (except when absorbance is 0.1 or less) is defined as the tangent to the fall of the absorption spectrum close to the long-wavelength region.

The local maximum absorbance of 0.2 or less is not counted as the above-mentioned local maximum absorbance closest to the long-wavelength region.

Measurement of Maximum Fluorescence Peak Wavelength (FL-Peak) A measurement target compound was dissolved in toluene at a concentration of 4.9×10⁻⁶ mol/L to prepare a toluene solution.

Using a fluorescence spectrometer (spectrophotofluorometer F-7000 produced by Hitachi High-Tech Science Corporation), the toluene solution was excited at 390 nm, where a maximum fluorescence peak wavelength A (unit: nm) was measured.

The maximum fluorescence peak wavelength A of the compound BD was 455 nm.

### Synthesis Examples

### Synthesis Example 1: Synthesis of BH1-1

The compound BH1-1 was synthesized according to the following synthesis scheme.

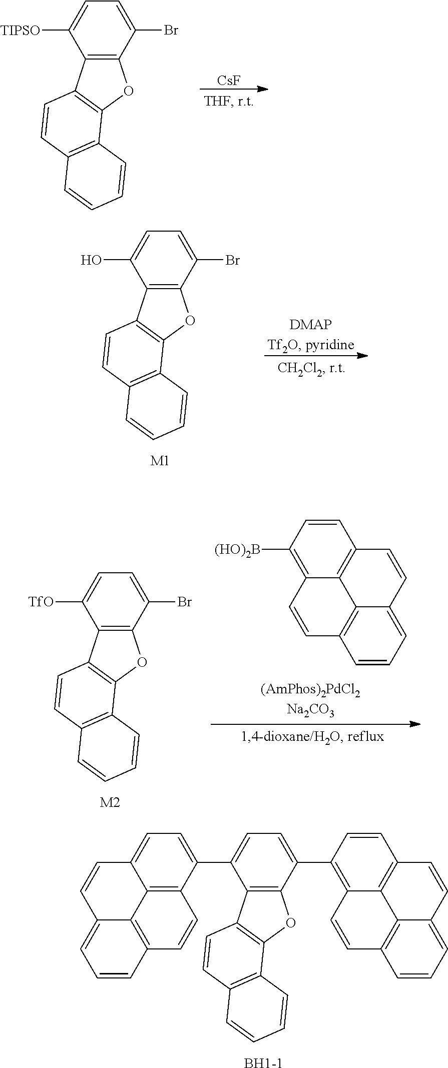

### (1) Synthesis of 10-Bromonaphtho[1,2-b]benzofuran-7-ol (Intermediate M1)

[(10-Bromonaphtho[1,2-b]benzofuran-7-yl)oxy]triisopropylsilane (2.26 g), cesium fluoride (1.80 g), and tetrahydrofuran (50 mL) were put in a flask and then stirred at room temperature for 5 hours.

After completion of the reaction, 0.2 mol/L hydrochloric acid was added to the solution under ice-cooling to neutralize the solution.

Water was added to the solution, and the resulting solution was extracted with ethyl acetate. After the organic layer was dried over anhydrous sodium sulfate, the solvent was distilled away, and the slurry was filtered.

The solid was washed with dichloromethane to obtain a white solid of 10-bromonaphtho[1,2-b]benzofuran-7-ol (1.47 g, yield 98%).

### (2) Synthesis of 10-Bromonaphtho[1,2-b]benzofuran-7-yl Trifluoromethanesulfonate (Intermediate M2)

10-Bromonaphtho[1,2-b]benzofuran-7-ol (intermediate M1) (1.47 g) and dichloromethane (40 mL) were put in a flask, and under ice-cooling, N,N-dimethyl-4-aminopyridine (0.58 g) and pyridine (0.40 mL) were added. Subsequently, trifluoromethanesulfonic acid anhydride (1.30 mL) was added dropwise, and stirring was performed for 4 hours while the temperature was increased to room temperature.

After completion of the reaction, saturated aqueous sodium bicarbonate was added to the solution under ice-cooling.

The solution was extracted with dichloromethane. After the organic layer was dried over anhydrous sodium sulfate, the solvent was distilled away, and the residue was purified by silica-gel column chromatography to obtain a white solid of 10-bromonaphtho[1,2-b]benzofuran-7-yl trifluoromethanesulfonate (1.50 g, yield 72%).

### (3) Synthesis of 7,10-Di(pyren-1-yl)naphtho[1,2-b]benzofuran (Compound BH1-1)

10-Bromonaphtho[1,2-b]benzofuran-7-yl trifluoromethanesulfonate (intermediate M2) (1.0 g), pyren-1-ylboronic acid (1.2 g), bis[di-tert-butyl(4-dimethylaminophenyl)phosphine]dichloropalladium (II) (0.20 g), sodium carbonate (0.50 g), 1,4-dioxane (20 mL), and ion-exchanged water (2 mL) were added to a flask and heated to reflux with stirring for 18 hours in an argon atmosphere.

After completion of the reaction, the solution was allowed to cool to room temperature, and a sufficient amount of water was added.

A solid was collected by filtration and washed with methanol.

The solid was thermally melted in toluene and subjected to silica-gel short column chromatography.

The solvent was distilled away to obtain a solid.

The solid obtained was recrystallized from toluene to obtain a light yellow solid of 7,10-di(pyren-1-yl)naphtho[1,2-b]benzofuran (compound BH1-1) (406 mg, yield 29%).

Mass spectral analysis showed a molecular weight of 618.74 and m/e=619; that is, the light yellow solid was identified as the target compound.

### Synthesis Example 2: Synthesis of BH1-2

A compound BH1-2 was synthesized according to the following synthesis scheme.

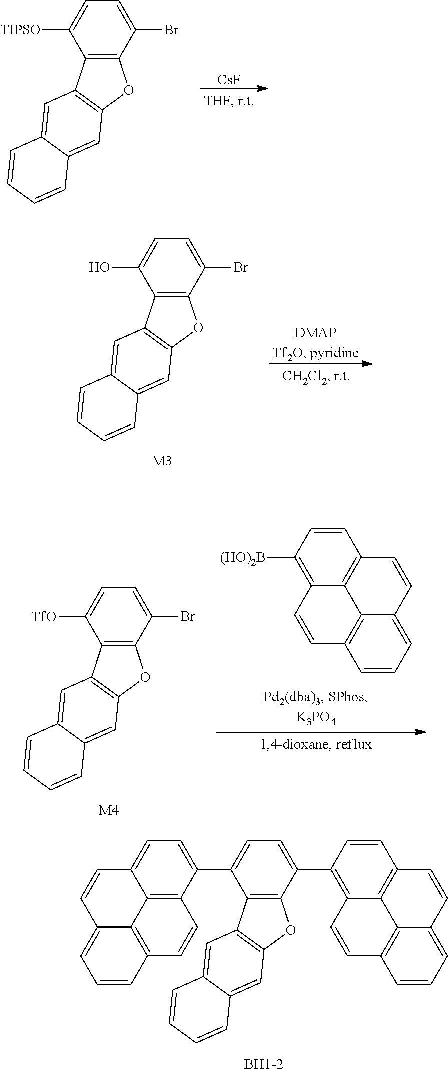

### (1) Synthesis of 4-Bromonaphtho[2,3-b]benzofuran-1-ol (Intermediate M3)

[(4-Bromonaphtho[2,3-b]benzofuran-1-yl)oxy]triisopropylsilane (5.00 g), cesium fluoride (4.00 g), and tetrahydrofuran (100 mL) were put in a flask and then stirred at room temperature for 5 hours.

After completion of the reaction, 0.2 mol/L hydrochloric acid was added to the solution under ice-cooling to neutralize the solution.

Water was added to the solution, and the resulting solution was extracted with ethyl acetate. After the organic layer was dried over anhydrous sodium sulfate, the solvent was distilled away, and the slurry was filtered.

The solid was washed with dichloromethane to obtain a white solid of 4-bromonaphtho[2,3-b]benzofuran-1-ol (3.20 g, yield 96%).

### (2) Synthesis of 4-Bromonaphtho[2,3-b]benzofuran-1-yl Trifluoromethanesulfonate (Intermediate M4)

4-Bromonaphtho[2,3-b]benzofuran-1-ol (intermediate M3) (3.1 g) and dichloromethane (100 mL) were put in a flask, and under ice-cooling, N,N-dimethyl-4-aminopyridine (1.21 g) and pyridine (1.6 mL) were added. Subsequently, trifluoromethanesulfonic acid anhydride (2.0 mL) was added dropwise, and stirring was performed for 4 hours while the temperature was increased to room temperature.

After completion of the reaction, saturated aqueous sodium bicarbonate was added to the solution under ice-cooling.

The solution was extracted with dichloromethane. After the organic layer was dried over anhydrous sodium sulfate, the solvent was distilled away, and the residue was purified by silica-gel column chromatography to obtain a white solid of 4-bromonaphtho[2,3-b]benzofuran-1-yl trifluoromethanesulfonate (3.57 g, yield 81%).

### (3) Synthesis of 1,4-Di(pyren-1-yl)naphtho[2,3-b]benzofuran (Compound BH1-2)

4-Bromonaphtho[2,3-b]benzofuran-1-yl trifluoromethanesulfonate (intermediate M4) (1.70 g), pyren-1-ylboronic acid (1.97 g), 2-dicyclohexylphosphino-2′,6′-dimethoxybiphenyl (0.10 g), tris(dibenzylideneacetone)dipalladium(0) (54 mg), tripotassium phosphate (1.62 g), and 1,4-dioxane (80 mL) were added to a flask and heated to reflux with stirring for 6 hours in an argon atmosphere.

After completion of the reaction, the solution was allowed to cool to room temperature, and a sufficient amount of water was added.

A solid was collected by filtration and washed with methanol.

The solid was thermally melted in toluene and subjected to silica-gel short column chromatography.

The solvent was distilled away to obtain a solid.

The solid obtained was recrystallized from toluene to obtain a light yellow solid of 1,4-di(pyren-1-yl)naphtho[2,3-b]benzofuran (compound BH1-2) (945 mg, yield 40%).

Mass spectral analysis showed a molecular weight of 618.74 and m/e=619; that is, the light yellow solid was identified as the target compound.

### Synthesis Example 3: Synthesis of BH1-3

A compound BH1-3 was synthesized according to the following synthesis scheme.

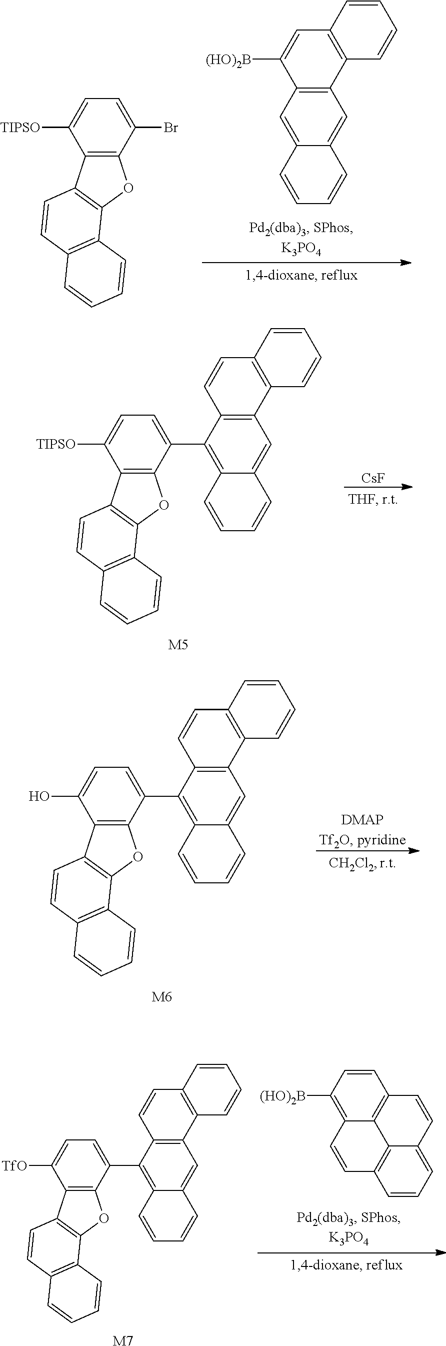

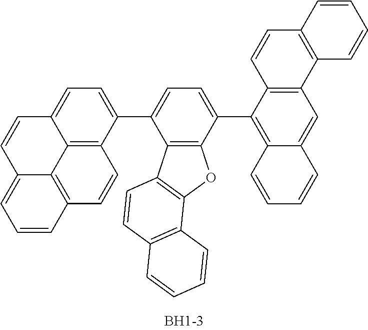 [SMILES: c1ccc2c(-c3ccc(-c4ccc5ccc6cccc7ccc4c5c67)c4c3oc3c5ccccc5ccc34)c3ccc4ccccc4c3cc2c1]

### (1) Synthesis of Triisopropyl{[4-(benzo[a]anthracen-7-yl)naphtho[2,3-b]benzofuran-1-yl]oxy}silane (Intermediate M5)

[(4-Bromonaphtho[2,3-b]benzofuran-1-yl)oxy]triisopropylsilane (5.00 g), benzo[a]anthracen-7-ylboronic acid (4.35 g), 2-dicyclohexylphosphino-2′,6′-dimethoxybiphenyl (0.30 g), tris(dibenzylideneacetone)dipalladium(0) (0.15 g), tripotassium phosphate (4.50 g), and 1,4-dioxane (120 mL) were added to a flask and heated to reflux with stirring for 6 hours in an argon atmosphere.

After completion of the reaction, the solution was allowed to cool to room temperature, and a sufficient amount of water was added.

A solid was collected by filtration and washed with methanol.

The solid was thermally melted in toluene and subjected to silica-gel short column chromatography.

The solvent was distilled away to obtain a solid.

The solid was purified by silica-gel column chromatography to obtain a white solid of triisopropyl{[4-(benzo[a]anthracen-7-yl)naphtho[2,3-b]benzofuran-1-yl]oxy}silane (4.65 g, yield 71%).

### (2) Synthesis of 4-(Benzo[a]anthracen-7-yl)naphtho[2,3-b]benzofuran-1-ol (Intermediate M6)

Triisopropyl{[4-(benzo[a]anthracen-7-yl)naphtho[2,3-b]benzofuran-1-yl]oxy}silane (intermediate M5) (4.60 g), cesium fluoride (2.83 g), and tetrahydrofuran (80 mL) were put in a flask and then stirred at room temperature for 5 hours.

After completion of the reaction, 0.2 mol/L hydrochloric acid was added to the solution under ice-cooling to neutralize the solution.

Water was added to the solution, and the resulting solution was extracted with dichloromethane. After the organic layer was dried over anhydrous sodium sulfate, the solvent was distilled away, and the slurry was filtered.

The solid was washed with dichloromethane to obtain a white solid of 4-(benzo[a]anthracen-7-yl)naphtho[2,3-b]benzofuran-1-ol (2.92 g, yield 85%).

### (3) Synthesis of 4-(Benzo[a]anthracen-7-yl)naphtho[2,3-b]benzofuran-1-yl Trifluoromethanesulfonate (Intermediate M7)

4-(Benzo[a]anthracen-7-yl)naphtho[2,3-b]benzofuran-1-ol (intermediate M6) (2.9 g) and dichloromethane (130 mL) were put in a flask, and under ice-cooling, N,N-dimethyl-4-aminopyridine (0.77 g) and pyridine (1.0 mL) were added.

Subsequently, trifluoromethanesulfonic acid anhydride (1.3 mL) was added dropwise, and stirring was performed for 5 hours while the temperature was increased to room temperature.

After completion of the reaction, saturated aqueous sodium bicarbonate was added to the solution under ice-cooling.

The solution was extracted with dichloromethane. After the organic layer was dried over anhydrous sodium sulfate, the solvent was distilled away, and the residue was purified by silica-gel column chromatography to obtain a white solid of 4-(benzo[a]anthracen-7-yl)naphtho[2,3-b]benzofuran-1-yl trifluoromethanesulfonate (3.30 g, yield 88%).

### (4) Synthesis of 1-(Pyren-1-yl)-4-(benzo[a]anthracen-7-yl)naphtho[2,3-b]benzofuran (Compound BH1-3)

4-(Benzo[a]anthracen-7-yl)naphtho[2,3-b]benzofuran-1-yl trifluoromethanesulfonate (intermediate M7) (1.70 g), pyren-1-ylboronic acid (0.75 g), 2-dicyclohexylphosphino-2′,6′-dimethoxybiphenyl (81 mg), tris(dibenzylideneacetone)dipalladium(0) (41 mg), tripotassium phosphate (0.91 g), and 1,4-dioxane (50 mL) were added to a flask and heated to reflux with stirring for 6 hours in an argon atmosphere.

After completion of the reaction, the solution was allowed to cool to room temperature, and a sufficient amount of water was added.

A solid was collected by filtration and washed with methanol.

The solid was thermally melted in toluene and subjected to silica-gel short column chromatography.

The solvent was distilled away to obtain a solid.

The solid obtained was recrystallized from toluene to obtain a light yellow solid of 1-(pyren-1-yl)-4-(benzo[a]anthracen-7-yl)naphtho[2,3-b]benzofuran (compound BH1-3) (810 mg, yield 44%).

Mass spectral analysis showed a molecular weight of 644.77 and m/e=645; that is, the light yellow solid was identified as the target compound.

### Synthesis Example 4: Synthesis of BH1-4

A compound BH1-4 was synthesized according to the following synthesis scheme.

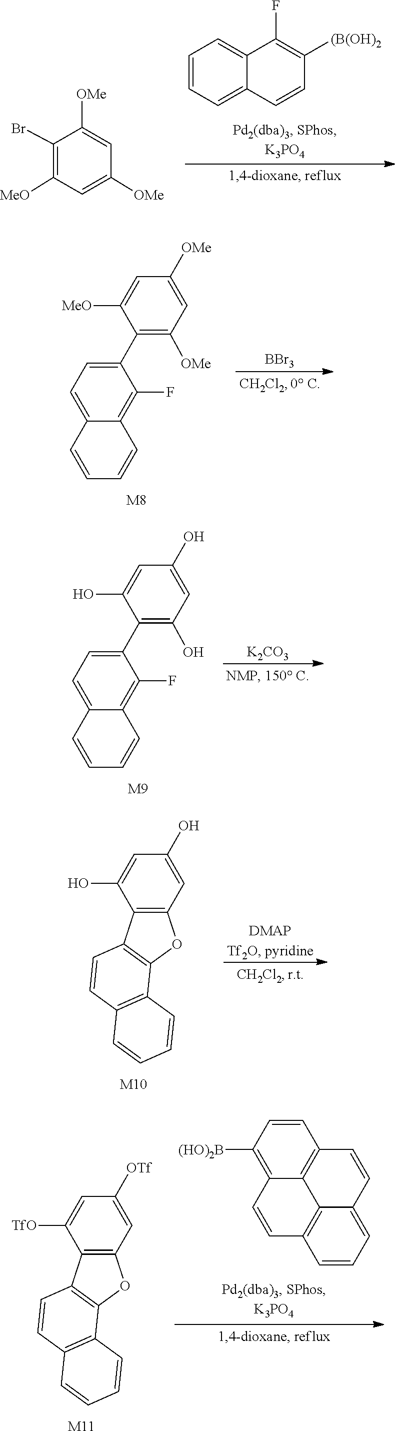

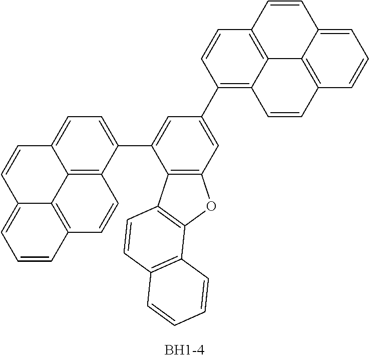 [SMILES: c1ccc2c(c1)ccc1c2oc2cc(-c3ccc4ccc5cccc6ccc3c4c56)cc(-c3ccc4ccc5cccc6ccc3c4c56)c21]

### (1) Synthesis of 1-Fluoro-2-(2,4,6-trimethoxyphenyl)naphthalene (Intermediate M8)

2-Bromo-1,3,5-trimethoxybenzene (5.00 g), (1-fluoronaphthalen-2-yl)boronic acid (4.61 g), 2-dicyclohexylphosphino-2′,6′-dimethoxybiphenyl (0.57 g), tris(dibenzylideneacetone)dipalladium(0) (0.29 g), tripotassium phosphate (6.43 g), and 1,4-dioxane (200 mL) were added to a flask and heated to reflux with stirring for 5 hours in an argon atmosphere.

After completion of the reaction, the solution was allowed to cool to room temperature, and a sufficient amount of water was added.

The dioxane was distilled away, and the solution was extracted with dichloromethane. After the organic layer was dried over anhydrous sodium sulfate, the solvent was distilled away, and the residue was purified by silica-gel column chromatography to obtain a white solid of 1-fluoro-2-(2,4,6-trimethoxyphenyl)naphthalene (5.90 g, yield 93%).

### (2) Synthesis of 2-(1-Fluoronaphthalen-2-yl)benzene-1,3,5-triol (Intermediate M9)

1-Fluoro-2-(2,4,6-trimethoxyphenyl)naphthalene (intermediate M8) (4.90 g) and dichloromethane (150 mL) were added to a flask and ice-cooled in an argon atmosphere. A 1.0 mol/L boron tribromide dichloromethane solution (95 mL) was added dropwise, and stirring was performed for 5 hours at room temperature.

After completion of the reaction, under ice-cooling, cold water was added dropwise to the solution, and the resulting solution was extracted with dichloromethane. After the organic layer was dried over anhydrous sodium sulfate, the solvent was distilled away, and the residue was purified by silica-gel column chromatography to obtain a white solid of 2-(1-fluoronaphthalen-2-yl)benzene-1,3,5-triol (3.41 g, yield 80%).

### (3) Synthesis of Naphtho[1,2-b]benzofuran-7,9-diol (Intermediate M10)

2-(1-Fluoronaphthalen-2-yl)benzene-1,3,5-triol (intermediate M9) (2.9 g), potassium carbonate (2.23 g), and N-methyl-2-pyrrolidone (210 mL) were added to a flask and heated with stirring for 5 hours at 150 degrees C. in an argon atmosphere.

After completion of the reaction, the reaction solution was allowed to cool to room temperature, and 500 mL of water was added. Furthermore, the solution was adjusted to pH 3 with dilute hydrochloric acid.

The solid precipitated was collected by filtration and purified by silica-gel column chromatography to obtain a white solid of naphtho[1,2-b]benzofuran-7,9-diol (1.71 g, yield 64%).

### (4) Synthesis of Naphtho[1,2-b]benzofuran-7,9-diyl bis(trifluoromethanesulfonate) (Intermediate M11)

Naphtho[1,2-b]benzofuran-7,9-diol (intermediate M10) (1.6 g) and dichloromethane (130 mL) were put in a flask, and under ice-cooling, N,N-dimethyl-4-aminopyridine (0.78 g) and pyridine (1.3 mL) were added. Subsequently, trifluoromethanesulfonic acid anhydride (2.6 mL) was added dropwise, and stirring was performed for 5 hours while the temperature was increased to room temperature.

After completion of the reaction, saturated aqueous sodium bicarbonate was added to the solution under ice-cooling.

The solution was extracted with dichloromethane. After the organic layer was dried over anhydrous sodium sulfate, the solvent was distilled away, and the residue was purified by silica-gel column chromatography to obtain a white solid of naphtho[1,2-b]benzofuran-7,9-diyl bis(trifluoromethanesulfonate) (2.55 g, yield 78%).

### (5) Synthesis of 7,9-Di(pyren-1-yl)naphtho[1,2-b]benzofuran (Compound BH1-4)

Naphtho[1,2-b]benzofuran-7,9-diyl bis(trifluoromethanesulfonate) (intermediate M11) (1.20 g), pyren-1-ylboronic acid (1.20 g), 2-dicyclohexylphosphino-2′,6′-dimethoxybiphenyl (132 mg), tris(dibenzylideneacetone)dipalladium(0) (66 mg), tripotassium phosphate (1.24 g), and 1,4-dioxane (50 mL) were added to a flask and heated to reflux with stirring for 6 hours in an argon atmosphere.

After completion of the reaction, the solution was allowed to cool to room temperature, and a sufficient amount of water was added.

A solid was collected by filtration and washed with methanol.

The solid was thermally melted in toluene and subjected to silica-gel short column chromatography.

The solvent was distilled away to obtain a solid.

The solid obtained was recrystallized from toluene to obtain a light yellow solid of 7,9-di(pyren-1-yl)naphtho[1,2-b]benzofuran (compound BH1-4) (0.95 g, yield 66%).

Mass spectral analysis showed a molecular weight of 618.74 and m/e=619; that is, the light yellow solid was identified as the target compound.

What is claimed is:

1. A compound represented by a formula (1) below, 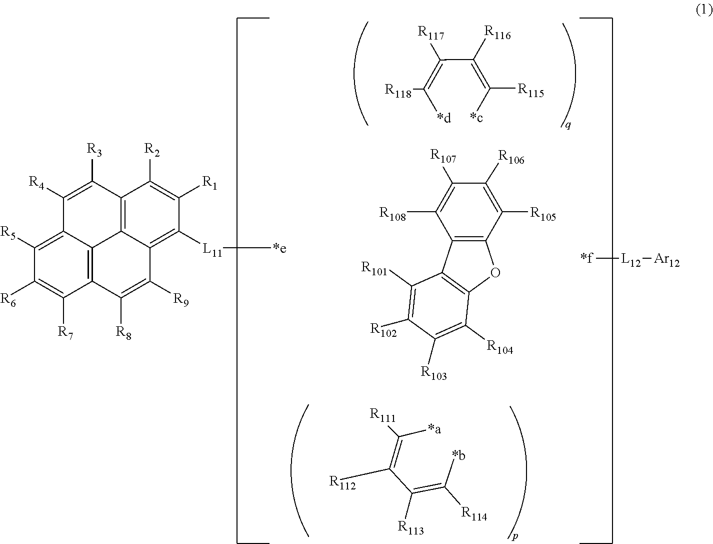
where, in the formula (1):
R₁ to R₉, R₁₀₁ to R₁₀₈, and R₁₁₁ to R₁₁₈ are each independently a hydrogen atom, a substituted or unsubstituted aryl group having 6 to 20 ring carbon atoms, or a substituted or unsubstituted heterocyclic group having 5 to 21 ring atoms;
Ar₁₂ is a substituted or unsubstituted aryl group having 10 to 30 ring carbon atoms, or a substituted or unsubstituted heterocyclic group having 9 to 31 ring atoms;
the substituted aryl group represented by Ar₁₂ has or does not have, as a substituent, at least one group selected from the group consisting of a substituted or unsubstituted aryl group having 6 to 20 ring carbon atoms and a substituted or unsubstituted heterocyclic group having 5 to 21 ring atoms;
the substituted heterocyclic group represented by Ar₁₂ has or does not have, as a substituent, at least one selected from the group consisting of a substituted or unsubstituted aryl group having 6 to 20 ring carbon atoms and a substituted or unsubstituted heterocyclic group having 5 to 21 ring atoms;
L₁₁ and L₁₂ are each independently a single bond, a substituted or unsubstituted arylene group having 6 to 10 ring carbon atoms, or a substituted or unsubstituted divalent heterocyclic group having 5 to 13 ring atoms;
p is 0 or 1;
q is 0 or 1; and
p+q is 1 or 2,
when p is 1, one of R₁₀₁ and R₁₀₂, R₁₀₂ and R₁₀₃, or R₁₀₃ and R₁₀₄ is a single bond bonded to *a, and the other of R₁₀₁ and R₁₀₂, R₁₀₂ and R₁₀₃, or R₁₀₃ and R₁₀₄ is a single bond bonded to *b;
when q is 0, of two selected from R₁₀₅ to R₁₀₈, one is a single bond bonded to *e, and the other is a single bond bonded to *f; and
when q is 1, one of R₁₀₅ and R₁₀₆, R₁₀₆ and R₁₀₇, or R₁₀₇ and R₁₀₈ is a single bond bonded to *c, and the other of R₁₀₅ and R₁₀₆, R₁₀₆ and R₁₀₇, or R₁₀₇ and R₁₀₈ is a single bond bonded to *d, and of two selected from R₁₀₅ to R₁₀₈ not being the single bonds bonded to *c and *d and R₁₁₅ to R₁₁₈, one is a single bond bonded to *e, and the other is a single bond bonded to *f.

2. The compound according to claim 1, wherein
the compound represented by the formula (1) is represented by a formula (10) below, 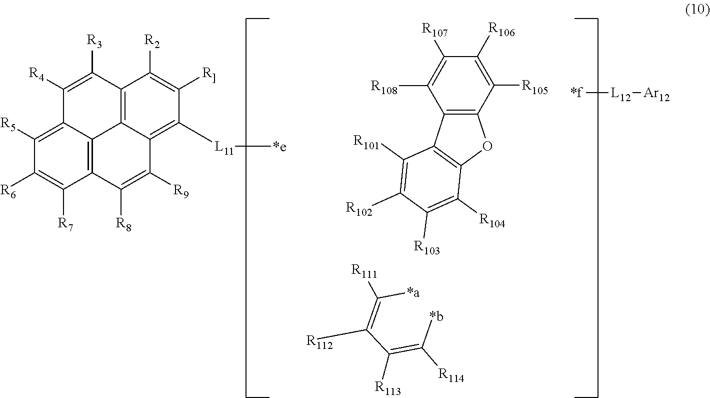
where, in the formula (10), R₁ to R₉, R₁₀₁ to R₁₀₈, R₁₁₁ to R₁₁₄, Ar₁₂, L₁₁, L₁₂, *a, *b, *e, and *f are as defined in the formula (1).

3. The compound according to claim 2, wherein
the compound represented by the formula (1) is represented by a formula (11) below, 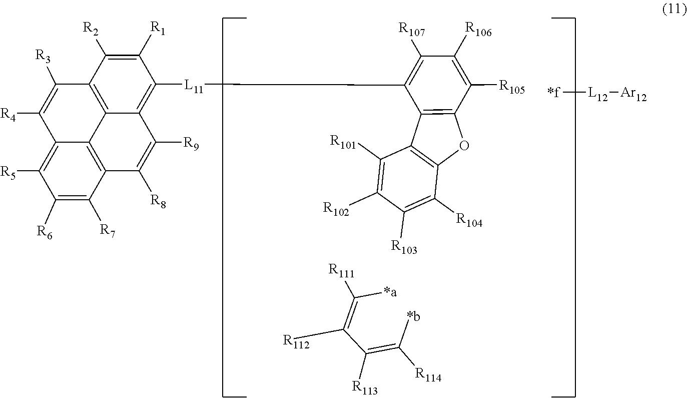
where, in the formula (11), R₁ to R₉, R₁₀₁ to R₁₀₇, R₁₁₁ to R₁₁₄, Ar₁₂, L₁₁, L₁₂, *a, *b, and *f are as defined in the formula (1).

4. The compound according to claim 2, wherein
in the compound represented by the formula (1), R₁₀₅ or R₁₀₆ is a single bond bonded to *f.

5. The compound according to claim 1, wherein
in the compound represented by the formula (1), one of R₁₀₂ and R₁₀₃ is a single bond bonded to *a, and the other of R₁₀₂ and R₁₀₃ is a single bond bonded to *b.

6. The compound according to claim 1, wherein
in the compound represented by the formula (1), one of R₁₀₃ and R₁₀₄ is a single bond bonded to *a, and the other of R₁₀₃ and R₁₀₄ is a single bond bonded to *b.

7. The compound according to claim 1, wherein
in the compound represented by the formula (1), Ar₁₂ is an aryl group derived from fusion of four or less monocyclic rings.

8. The compound according to claim 1, wherein
in the compound represented by the formula (1), R₁ to R₉, R₁₀₁ to R₁₀₈ not being the single bonds, and R₁₁₁ to R₁₁₈ not being the single bonds are each independently a hydrogen atom or a substituted or unsubstituted aryl group having 6 to 10 ring carbon atoms.

9. The compound according to claim 1, wherein
in the compound represented by the formula (1), R₁ to R₉, R₁₀₁ to R₁₀₈ not being the single bonds, and R₁₁₁ to R₁₁₈ not being the single bonds are each a hydrogen atom.

10. The compound according to claim 1, wherein
in the compound represented by the formula (1), the aryl group represented by Ar₁₂ has, as a substituent, an aryl group having 6 to 10 ring carbon atoms.

11. The compound according to claim 1, wherein
in the compound represented by the formula (1), Ar₁₂ is an unsubstituted aryl group having 10 to 30 ring carbon atoms.

12. The compound according to claim 1, wherein
in the compound represented by the formula (1), L₁₁ is a single bond.

13. An organic electroluminescence device comprising:
an anode;
a cathode; and
an emitting zone provided between the anode and the cathode,
wherein the emitting zone comprises a first emitting layer comprising the compound according to claim 1 as a first host material.

14. The organic electroluminescence device according to claim 13, wherein
the emitting zone further comprises a second emitting layer;
the first emitting layer comprises the first host material and a first emitting compound;
the second emitting layer comprises a second host material and a second emitting compound;
the first host material and the second host material are mutually different; and
the first emitting compound and the second emitting compound are mutually the same or different.

15. The organic electroluminescence device according to claim 14, wherein
a triplet energy T₁(H1) of the first host material and a triplet energy T₁(H2) of the second host material satisfy a relationship of a numerical formula (Numerical Formula 1) below,
T₁(H1)>T₁(H2) (Numerical Formula 1).

16. The organic electroluminescence device according to claim 14, wherein
the first emitting compound and the second emitting compound are each independently a compound that emits light with a maximum peak wavelength of 500 nm or less.

17. The organic electroluminescence device according to claim 14, wherein
the first emitting layer is provided between the anode and the second emitting layer.

18. The organic electroluminescence device according to claim 14, wherein
the second host material is a second compound represented by a formula (2) below, 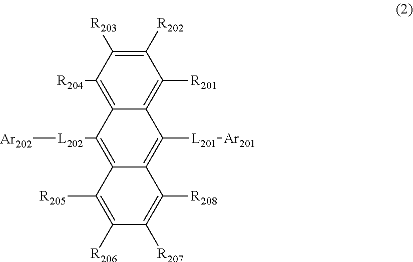
where, in the formula (2):
R₂₀₁ to R₂₀₈ are each independently a hydrogen atom, a substituted or unsubstituted alkyl group having 1 to 50 carbon atoms, a substituted or unsubstituted haloalkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a group represented by —S—(R₉₀₅), a group represented by —N(R₉₀₆)(R₉₀₇), a substituted or unsubstituted aralkyl group having 7 to 50 carbon atoms, a group represented by —C(═O)R₈₀₁, a group represented by —COOR₈₀₂, a halogen atom, a cyano group, a nitro group, a substituted or unsubstituted aryl group having 6 to 50 ring carbon atoms, or a substituted or unsubstituted heterocyclic group having 5 to 50 ring atoms;
L₂₀₁ and L₂₀₂ are each independently a single bond, a substituted or unsubstituted arylene group having 6 to 50 ring carbon atoms, or a substituted or unsubstituted divalent heterocyclic group having 5 to 50 ring atoms;
Ar₂₀₁ and Ar₂₀₂ are each independently a substituted or unsubstituted aryl group having 6 to 50 ring carbon atoms, or a substituted or unsubstituted heterocyclic group having 5 to 50 ring atoms;
in the second host material, R₉₀₁, R₉₀₂, R₉₀₃, R₉₀₄, R₉₀₅, R₉₀₆, R₉₀₇, R₈₀₁, and R₈₀₂ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different;
when a plurality of R₉₀₄ are present, the plurality of R₉₀₄ are mutually the same or different;
when a plurality of R₉₀₅ are present, the plurality of R₉₀₅ are mutually the same or different;
when a plurality of R₉₀₆ are present, the plurality of R₉₀₆ are mutually the same or different;
when a plurality of R₉₀₇ are present, the plurality of R₉₀₇ are mutually the same or different;
when a plurality of R₈₀₁ are present, the plurality of R₈₀₁ are mutually the same or different; and
when a plurality of R₈₀₂ are present, the plurality of R₈₀₂ are mutually the same or different.

19. The organic electroluminescence device according to claim 13, further comprising a hole transporting layer between the anode and the emitting zone, wherein
the hole transporting layer comprises a third compound represented by a formula (H1) below or a formula (H2) below, 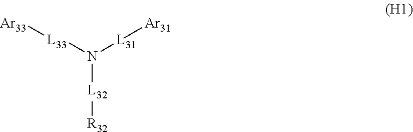
where, in the formula (H1):
L₃₁, L₃₂, and L₃₃ are each independently a single bond, or a substituted or unsubstituted arylene group having 6 to 18 ring carbon atoms;
Ar₃₁, Ar₃₂, and Ar₃₃ are each independently a substituted or unsubstituted aryl group having 6 to 30 ring carbon atoms, a substituted or unsubstituted heterocyclic group having 5 to 30 ring atoms, or a group represented by —Si(R_{C1})(R_{C2})(R_{C3});
R_{C1}, R_{C2}, and R_{C3} are each independently a substituted or unsubstituted aryl group having 6 to 30 ring carbon atoms;
when a plurality of R_{C1} are present, the plurality of R_{C1} are mutually the same or different;
when a plurality of R_{C2} are present, the plurality of R_{C2} are mutually the same or different; and
when a plurality of R_{C3} are present, the plurality of R_{C3} are mutually the same or different, 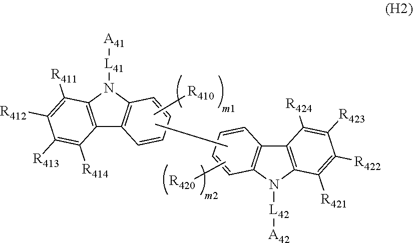
where, in the formula (H2):
A₄₁ and A₄₂ are each independently a substituted or unsubstituted aryl group having 6 to 30 ring carbon atoms, or a substituted or unsubstituted heterocyclic group having 5 to 30 ring atoms;
at least one combination of adjacent two or more of R₄₁₀ to R₄₁₄ are mutually bonded to form a substituted or unsubstituted monocyclic ring, mutually bonded to form a substituted or unsubstituted fused ring, or not mutually bonded;
at least one combination of adjacent two or more of R₄₂₀ to R₄₂₄ are mutually bonded to form a substituted or unsubstituted monocyclic ring, mutually bonded to form a substituted or unsubstituted fused ring, or not mutually bonded;
R₄₁₀ to R₄₁₄ and R₄₂₀ to R₄₂₄ forming neither the substituted or unsubstituted monocyclic ring nor the substituted or unsubstituted fused ring are each independently a hydrogen atom, a cyano group, a substituted or unsubstituted alkyl group having 1 to 50 carbon atoms, a substituted or unsubstituted alkenyl group having 2 to 50 carbon atoms, a substituted or unsubstituted alkynyl group having 2 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a halogen atom, a nitro group, a substituted or unsubstituted aryl group having 6 to 50 ring carbon atoms, or a substituted or unsubstituted heterocyclic group having 5 to 50 ring atoms;
m1 is 3, and three R₄₁₀ are mutually the same or different;
m2 is 3, and three R₄₂₀ are mutually the same or different;
L₄₁ and L₄₂ are each independently a single bond, a substituted or unsubstituted arylene group having 6 to 30 ring carbon atoms, or a substituted or unsubstituted divalent heterocyclic group having 5 to 30 ring atoms;
in the third compound represented by the formula (H2), R₉₀₁, R₉₀₂, R₉₀₃, and R₉₀₄ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different; and
when a plurality of R₉₀₄ are present, the plurality of R₉₀₄ are mutually the same or different.

20. The organic electroluminescence device according to claim 13, further comprising an electron transporting layer between the emitting zone and the cathode, and
the electron transporting layer comprises a fourth compound represented by a formula (E1) below, 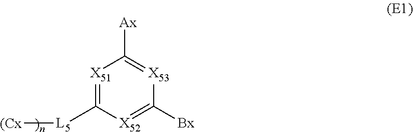
where, in the formula (E1):
X₅₁, X₅₂, and X₅₃ are each independently a nitrogen atom or CR₅;
at least one of X₅₁, X₅₂, or X₅₃ is a nitrogen atom;
R₅ is a hydrogen atom, a cyano group, a substituted or unsubstituted alkyl group having 1 to 50 carbon atoms, a substituted or unsubstituted cycloalkyl group having 3 to 50 ring carbon atoms, a group represented by —Si(R₉₀₁)(R₉₀₂)(R₉₀₃), a group represented by —O—(R₉₀₄), a substituted or unsubstituted aryl group having 6 to 50 ring carbon atoms, or a substituted or unsubstituted heterocyclic group having 5 to 50 ring atoms;
Ax is a substituted or unsubstituted aryl group having 6 to 18 ring carbon atoms, or a substituted or unsubstituted heterocyclic group having 5 to 13 ring atoms;
Bx is a substituted or unsubstituted aryl group having 6 to 18 ring carbon atoms, or a substituted or unsubstituted heterocyclic group having 5 to 13 ring atoms;
L₅ is a single bond, a substituted or unsubstituted (n+1)-valent aromatic hydrocarbon ring group having 6 to 18 ring carbon atoms, a substituted or unsubstituted (n+1)-valent heterocyclic group having 5 to 13 ring atoms, or an (n+1)-valent group derived from bonding of two or three selected from the group consisting of substituted or unsubstituted aromatic hydrocarbon ring groups having 6 to 18 ring carbon atoms and substituted or unsubstituted heterocyclic groups having 5 to 13 ring atoms;
n is 1, 2, or 3, and when n is 2 or 3, L₅ is not a single bond;
each Cx independently represents a substituted or unsubstituted aryl group having 6 to 30 ring carbon atoms, or a substituted or unsubstituted heterocyclic group having 5 to 60 ring atoms;
when a plurality of Cx are present, the plurality of Cx are mutually the same or different;
in the fourth compound, R₉₀₁, R₉₀₂, R₉₀₃, and R₉₀₄ are each independently a hydrogen atom, a substituted or unsubstituted alkyl group having 1 to 50 carbon atoms, a substituted or unsubstituted cycloalkyl group having 3 to 50 ring carbon atoms, a substituted or unsubstituted aryl group having 6 to 50 ring carbon atoms, or a substituted or unsubstituted heterocyclic group having 5 to 50 ring atoms;
when a plurality of R₉₀₁ are present, the plurality of R₉₀₁ are mutually the same or different;
when a plurality of R₉₀₂ are present, the plurality of R₉₀₂ are mutually the same or different;
when a plurality of R₉₀₃ are present, the plurality of R₉₀₃ are mutually the same or different; and
when a plurality of R₉₀₄ are present, the plurality of R₉₀₄ are mutually the same or different.

21. An electronic device comprising the organic electroluminescence device according to claim 13.
